SEQUENCE LISTING (544 sequences)

SEQ ID NO: 1 - DNA, 25 bp, Artificial Sequence
  Description of Artificial Sequence Synthetic primer
ttggttgtttggtaggggtagttat

SEQ ID NO: 2 - DNA, 28 bp, Artificial Sequence
  Description of Artificial Sequence Synthetic primer
tgaaatgtttttaatgatttagttgatg

SEQ ID NO: 3 - DNA, 29 bp, Artificial Sequence
  Description of Artificial Sequence Synthetic primer
ggggtgttgtagaattttttttagtttaa

SEQ ID NO: 4 - DNA, 24 bp, Artificial Sequence
  Description of Artificial Sequence Synthetic primer
ggggttagggtttatttttgggta

SEQ ID NO: 5 - DNA, 25 bp, Artificial Sequence
  Description of Artificial Sequence Synthetic primer
ttgttaatggtgatgatttggttat

SEQ ID NO: 6 - DNA, 24 bp, Artificial Sequence
  Description of Artificial Sequence Synthetic primer
ggaagttgggatttgagttggttt

SEQ ID NO: 7 - DNA, 25 bp, Artificial Sequence
  Description of Artificial Sequence Synthetic primer
ttttttttggttttgttttggtttg

SEQ ID NO: 8 - DNA, 25 bp, Artificial Sequence
  Description of Artificial Sequence Synthetic primer
gggagtggttgaaatttaagttgag

SEQ ID NO: 9 - DNA, 30 bp, Artificial Sequence
  Description of Artificial Sequence Synthetic primer
ggttttgttgttgtagatttgttttattta

SEQ ID NO: 10 - DNA, 25 bp, Artificial Sequence
  Description of Artificial Sequence Synthetic primer
tttttgtgggttttagagaaagttt

SEQ ID NO: 11 - DNA, 24 bp, Artificial Sequence
  Description of Artificial Sequence Synthetic primer
ggggagttttttattttaattggg

SEQ ID NO: 12 - DNA, 25 bp, Artificial Sequence
  Description of Artificial Sequence Synthetic primer
tttatttttagggaaagagggaggg

SEQ ID NO: 13 - DNA, 25 bp, Artificial Sequence
  Description of Artificial Sequence Synthetic primer
agggaggtgggtagttttgtaggag

SEQ ID NO: 14 - DNA, 29 bp, Artificial Sequence
  Description of Artificial Sequence Synthetic primer
gggttttttttattgtaggttgaaggtat

SEQ ID NO: 15 - DNA, 24 bp, Artificial Sequence
  Description of Artificial Sequence Synthetic primer
gttggggaggatttagagggagat

SEQ ID NO: 16 - DNA, 25 bp, Artificial Sequence
  Description of Artificial Sequence Synthetic primer
tttggattttgtggttgtttttttt

SEQ ID NO: 17 - DNA, 27 bp, Artificial Sequence
  Description of Artificial Sequence Synthetic primer
aagttggaggagtaggtttagtagata

SEQ ID NO: 18 - DNA, 26 bp, Artificial Sequence
  Description of Artificial Sequence Synthetic primer
catccagaggaggtctgtgtggtgtg

SEQ ID NO: 19 - DNA, 30 bp, Artificial Sequence
  Description of Artificial Sequence Synthetic primer
ggtgtttagagaaattttagaaagttggat

SEQ ID NO: 20 - DNA, 30 bp, Artificial Sequence
  Description of Artificial Sequence Synthetic primer
ttttttaggatataggttattttttgaagg

SEQ ID NO: 21 - DNA, 25 bp, Artificial Sequence
  Description of Artificial Sequence Synthetic primer
tttttttgatttattttgaggtttt

SEQ ID NO: 22 - DNA, 30 bp, Artificial Sequence
  Description of Artificial Sequence Synthetic primer
gggagatagaatttatttggtttatttata

SEQ ID NO: 23 - DNA, 26 bp, Artificial Sequence
  Description of Artificial Sequence Synthetic primer
ttaggagtgtttgggtatggttagta

SEQ ID NO: 24 - DNA, 25 bp, Artificial Sequence
  Description of Artificial Sequence Synthetic primer
gattgggtttgaatgtaattgaaag

SEQ ID NO: 25 - DNA, 25 bp, Artificial Sequence
  Description of Artificial Sequence Synthetic primer
gttaggggttttttttgtttttttt

SEQ ID NO: 26 - DNA, 29 bp, Artificial Sequence
  Description of Artificial Sequence Synthetic primer
ggattggtgggaaaataagagagtagatt

SEQ ID NO: 27 - DNA, 26 bp, Artificial Sequence
  Description of Artificial Sequence Synthetic primer
gattttttttgttttatagggggatt

SEQ ID NO: 28 - DNA, 25 bp, Artificial Sequence
  Description of Artificial Sequence Synthetic primer
ccctgaggcagagggtgaggagtag

SEQ ID NO: 29 - DNA, 25 bp, Artificial Sequence
  Description of Artificial Sequence Synthetic primer
tgttttttaaattttttggagggat

SEQ ID NO: 30 - DNA, 28 bp, Artificial Sequence
  Description of Artificial Sequence Synthetic primer
ggttgaatgttagttttgaattaaaagt

SEQ ID NO: 31 - DNA, 29 bp, Artificial Sequence
  Description of Artificial Sequence Synthetic primer
taatggtagggttgggaaggtgtatatta

SEQ ID NO: 32 - DNA, 25 bp, Artificial Sequence
  Description of Artificial Sequence Synthetic primer
gggactctctgtgtggtgctgacag

SEQ ID NO: 33 - DNA, 30 bp, Artificial Sequence
  Description of Artificial Sequence Synthetic primer
gggttggaaaattttttttataattatttt

SEQ ID NO: 34 - DNA, 25 bp, Artificial Sequence
  Description of Artificial Sequence Synthetic primer
ttgggggagttttatttttggagat

SEQ ID NO: 35 - DNA, 25 bp, Artificial Sequence
  Description of Artificial Sequence Synthetic primer
aagggtttttgttgaagtgggttat

SEQ ID NO: 36 - DNA, 26 bp, Artificial Sequence
  Description of Artificial Sequence Synthetic primer
catgtagactctttgtggctggggag

SEQ ID NO: 37 - DNA, 30 bp, Artificial Sequence
  Description of Artificial Sequence Synthetic primer
tgtgtatttggattaattgttatatagttt

SEQ ID NO: 38 - DNA, 30 bp, Artificial Sequence
  Description of Artificial Sequence Synthetic primer
gggtttttatatattttttaggggaattga

SEQ ID NO: 39 - DNA, 24 bp, Artificial Sequence
  Description of Artificial Sequence Synthetic primer
gttaggaatgtggttttggggatt

SEQ ID NO: 40 - DNA, 24 bp, Artificial Sequence
  Description of Artificial Sequence Synthetic primer
ttttttttgggggtttttttgtgt

SEQ ID NO: 41 - DNA, 26 bp, Artificial Sequence
  Description of Artificial Sequence Synthetic primer
gggaaggggatatatgagggatttat

SEQ ID NO: 42 - DNA, 26 bp, Artificial Sequence
  Description of Artificial Sequence Synthetic primer
ggggtggtagttagagagtttgagag

SEQ ID NO: 43 - DNA, 29 bp, Artificial Sequence
  Description of Artificial Sequence Synthetic primer
gggttggaatttagttttagttttgttgt

SEQ ID NO: 44 - DNA, 30 bp, Artificial Sequence
  Description of Artificial Sequence Synthetic primer
gggtattggagaataaagatatttttaata

SEQ ID NO: 45 - DNA, 23 bp, Artificial Sequence
  Description of Artificial Sequence Synthetic primer
gggggtttttagttgatagaggg

SEQ ID NO: 46 - DNA, 20 bp, Artificial Sequence
  Description of Artificial Sequence Synthetic primer
ttgttgtttgggagggaggt

SEQ ID NO: 47 - DNA, 25 bp, Artificial Sequence
  Description of Artificial Sequence Synthetic primer
tgttgtgatttgggagaggtttaag

SEQ ID NO: 48 - DNA, 30 bp, Artificial Sequence
  Description of Artificial Sequence Synthetic primer
tttttatattaaagtatttgggatggtttt

SEQ ID NO: 49 - DNA, 28 bp, Artificial Sequence
  Description of Artificial Sequence Synthetic primer
gggagattagtatttaaagttggaggtt

SEQ ID NO: 50 - DNA, 27 bp, Artificial Sequence
  Description of Artificial Sequence Synthetic primer
ggtattttaggggaagttggtattttg

SEQ ID NO: 51 - DNA, 27 bp, Artificial Sequence
  Description of Artificial Sequence Synthetic primer
agtgttaggaatttagattttggtaat

SEQ ID NO: 52 - DNA, 28 bp, Artificial Sequence
  Description of Artificial Sequence Synthetic primer
gtttagagagagggattggaggtttaga

SEQ ID NO: 53 - DNA, 28 bp, Artificial Sequence
  Description of Artificial Sequence Synthetic primer
gttttttgtagttgtttgttgggttttg

SEQ ID NO: 54 - DNA, 25 bp, Artificial Sequence
  Description of Artificial Sequence Synthetic primer
ttttttgtttgttagggtttttttt

SEQ ID NO: 55 - DNA, 26 bp, Artificial Sequence
  Description of Artificial Sequence Synthetic primer
tgtattttttaatggttggtttgttt

SEQ ID NO: 56 - DNA, 25 bp, Artificial Sequence
  Description of Artificial Sequence Synthetic primer
ttggtttagggtaataggggttttg

SEQ ID NO: 57 - DNA, 24 bp, Artificial Sequence
  Description of Artificial Sequence Synthetic primer
tgatttttatagagtatgggtggg

SEQ ID NO: 58 - DNA, 30 bp, Artificial Sequence
  Description of Artificial Sequence Synthetic primer
attagagtatgatttaggtttttgatagtt

SEQ ID NO: 59 - DNA, 25 bp, Artificial Sequence
  Description of Artificial Sequence Synthetic primer
gttgtaggtggtttttttaaggatg

SEQ ID NO: 60 - DNA, 29 bp, Artificial Sequence
  Description of Artificial Sequence Synthetic primer
taggattttgttgaatgaatgattgaatt

SEQ ID NO: 61 - DNA, 25 bp, Artificial Sequence
  Description of Artificial Sequence Synthetic primer
ggggaggagattatttggttttttt

SEQ ID NO: 62 - DNA, 26 bp, Artificial Sequence
  Description of Artificial Sequence Synthetic primer
gggacctgggaaggagcataggacag

SEQ ID NO: 63 - DNA, 23 bp, Artificial Sequence
  Description of Artificial Sequence Synthetic primer
gggtttagggggaggagatttag

SEQ ID NO: 64 - DNA, 23 bp, Artificial Sequence
  Description of Artificial Sequence Synthetic primer
gaggagagttttttggggaaatg

SEQ ID NO: 65 - DNA, 25 bp, Artificial Sequence
  Description of Artificial Sequence Synthetic primer
gtaggtagtgtgttaggaagggggt

SEQ ID NO: 66 - DNA, 26 bp, Artificial Sequence
  Description of Artificial Sequence Synthetic primer
gattgttttggggtaataaaaagatt

SEQ ID NO: 67 - DNA, 23 bp, Artificial Sequence
  Description of Artificial Sequence Synthetic primer
tgggaaagagggaaaggtttttt

SEQ ID NO: 68 - DNA, 23 bp, Artificial Sequence
  Description of Artificial Sequence Synthetic primer
gtagttgggggatgtttggattt

SEQ ID NO: 69 - DNA, 23 bp, Artificial Sequence
  Description of Artificial Sequence Synthetic primer
agggttttggggatttattggag

SEQ ID NO: 70 - DNA, 25 bp, Artificial Sequence
  Description of Artificial Sequence Synthetic primer
tttaggttagttggggtattttggg

SEQ ID NO: 71 - DNA, 29 bp, Artificial Sequence
  Description of Artificial Sequence Synthetic primer
tttttttttagtgtttagtttagagtttg

SEQ ID NO: 72 - DNA, 30 bp, Artificial Sequence
  Description of Artificial Sequence Synthetic primer
tggttgatattttttgtgtaaaatatgttg

SEQ ID NO: 73 - DNA, 25 bp, Artificial Sequence
  Description of Artificial Sequence Synthetic primer
gggtattattggtttaatggggaag

SEQ ID NO: 74 - DNA, 30 bp, Artificial Sequence
  Description of Artificial Sequence Synthetic primer
ttttttttgtagttattttaggggaagtaa

SEQ ID NO: 75 - DNA, 25 bp, Artificial Sequence
  Description of Artificial Sequence Synthetic primer
ttttaggtttggaggttggttaggt

SEQ ID NO: 76 - DNA, 25 bp, Artificial Sequence
  Description of Artificial Sequence Synthetic primer
tggattttttttatttaggggtata

SEQ ID NO: 77 - DNA, 28 bp, Artificial Sequence
  Description of Artificial Sequence Synthetic primer
ttagaatggaagggtaagaggtttaaat

SEQ ID NO: 78 - DNA, 30 bp, Artificial Sequence
  Description of Artificial Sequence Synthetic primer
ttttttttattaattggaggagaattataa

SEQ ID NO: 79 - DNA, 26 bp, Artificial Sequence
  Description of Artificial Sequence Synthetic primer
tttagggttttagtggtggttattat

SEQ ID NO: 80 - DNA, 27 bp, Artificial Sequence
  Description of Artificial Sequence Synthetic primer
gagagaattttgtaggttaggggagag

SEQ ID NO: 81 - DNA, 27 bp, Artificial Sequence
  Description of Artificial Sequence Synthetic primer
gagagaattttgtaggttaggggagag

SEQ ID NO: 82 - DNA, 27 bp, Artificial Sequence
  Description of Artificial Sequence Synthetic primer
gaaggttggttttggtttttgagtaga

SEQ ID NO: 83 - DNA, 23 bp, Artificial Sequence
  Description of Artificial Sequence Synthetic primer
ttagtttttagggagtttggagt

SEQ ID NO: 84 - DNA, 25 bp, Artificial Sequence
  Description of Artificial Sequence Synthetic primer
gagtgggtgggtttagttaggtttg

SEQ ID NO: 85 - DNA, 24 bp, Artificial Sequence
  Description of Artificial Sequence Synthetic primer
tattagggggtttaggggttggtt

SEQ ID NO: 86 - DNA, 27 bp, Artificial Sequence
  Description of Artificial Sequence Synthetic primer
ggtagagtagaagggtttttgtttttt

SEQ ID NO: 87 - DNA, 22 bp, Artificial Sequence
  Description of Artificial Sequence Synthetic primer
tttttagggggaagggaggttt

SEQ ID NO: 88 - DNA, 25 bp, Artificial Sequence
  Description of Artificial Sequence Synthetic primer
tttaggtagaggagtggattggagt

SEQ ID NO: 89 - DNA, 26 bp, Artificial Sequence
  Description of Artificial Sequence Synthetic primer
gaggttattaggtgggattttttgag

SEQ ID NO: 90 - DNA, 21 bp, Artificial Sequence
  Description of Artificial Sequence Synthetic primer
ttggttgggttgttggaaggt

SEQ ID NO: 91 - DNA, 29 bp, Artificial Sequence
  Description of Artificial Sequence Synthetic primer
ggggtgttgtagaattttttttagtttaa

SEQ ID NO: 92 - DNA, 25 bp, Artificial Sequence
  Description of Artificial Sequence Synthetic primer
tattttgtttaggtaggaggttagg

SEQ ID NO: 93 - DNA, 27 bp, Artificial Sequence
  Description of Artificial Sequence Synthetic primer
tttttagtttaggtgggattatatggt

SEQ ID NO: 94 - DNA, 25 bp, Artificial Sequence
  Description of Artificial Sequence Synthetic primer
ttttggataagggaagttgtgtatt

SEQ ID NO: 95 - DNA, 25 bp, Artificial Sequence
  Description of Artificial Sequence Synthetic primer
ttttaaaggtttttgggtagtgatt

SEQ ID NO: 96 - DNA, 24 bp, Artificial Sequence
  Description of Artificial Sequence Synthetic primer
gttttttgtgggtgtggtttttta

SEQ ID NO: 97 - DNA, 27 bp, Artificial Sequence
  Description of Artificial Sequence Synthetic primer
tggtgttttataggtatttgggttgtg

SEQ ID NO: 98 - DNA, 26 bp, Artificial Sequence
  Description of Artificial Sequence Synthetic primer
tggaaagttttgatttttttgagttt

SEQ ID NO: 99 - DNA, 29 bp, Artificial Sequence
  Description of Artificial Sequence Synthetic primer
agtttgttaagttttattgggttttagtt

SEQ ID NO: 100 - DNA, 30 bp, Artificial Sequence
  Description of Artificial Sequence Synthetic primer
ggagtatatagaagttgtaggttaggaggt

SEQ ID NO: 101 - DNA, 27 bp, Artificial Sequence
  Description of Artificial Sequence Synthetic primer
gggtcctgaccttgattcctgccacag

SEQ ID NO: 102 - DNA, 29 bp, Artificial Sequence
  Description of Artificial Sequence Synthetic primer
tgggtttttgtatagattaaaaataaaaa

SEQ ID NO: 103 - DNA, 28 bp, Artificial Sequence
  Description of Artificial Sequence Synthetic primer
ggtagtttttgtttgaaattttagtttt

SEQ ID NO: 104 - DNA, 25 bp, Artificial Sequence
  Description of Artificial Sequence Synthetic primer
atatttatttggtgttgggtgtggg

SEQ ID NO: 105 - DNA, 30 bp, Artificial Sequence
  Description of Artificial Sequence Synthetic primer
gatggtttaagatttatttgttgggtaggt

SEQ ID NO: 106 - DNA, 24 bp, Artificial Sequence
  Description of Artificial Sequence Synthetic primer
gttggtttgggggtttttgattag

SEQ ID NO: 107 - DNA, 26 bp, Artificial Sequence
  Description of Artificial Sequence Synthetic primer
gggcctgtcttcagaagagaaaatgg

SEQ ID NO: 108 - DNA, 26 bp, Artificial Sequence
  Description of Artificial Sequence Synthetic primer
ttgtttttttatggaaatgaaggatt

SEQ ID NO: 109 - DNA, 25 bp, Artificial Sequence
  Description of Artificial Sequence Synthetic primer
gagatgttggtttttgtgggaagtt

SEQ ID NO: 110 - DNA, 30 bp, Artificial Sequence
  Description of Artificial Sequence Synthetic primer
ggggatagaggagtattgaaagttagttta

SEQ ID NO: 111 - DNA, 29 bp, Artificial Sequence
  Description of Artificial Sequence Synthetic primer
cctcagattgaggtcccagggccaaagga

SEQ ID NO: 112 - DNA, 23 bp, Artificial Sequence
  Description of Artificial Sequence Synthetic primer
gtagaattggggatttttggtgt

SEQ ID NO: 113 - DNA, 27 bp, Artificial Sequence
  Description of Artificial Sequence Synthetic primer
tttaaataaagtaaaggaatgggtttt

SEQ ID NO: 114 - DNA, 29 bp, Artificial Sequence
  Description of Artificial Sequence Synthetic primer
ttagtgggaatttttagttaggaagtgag

SEQ ID NO: 115 - DNA, 29 bp, Artificial Sequence
  Description of Artificial Sequence Synthetic primer
tcagtgggaatttccagccaggaagtgag

SEQ ID NO: 116 - DNA, 30 bp, Artificial Sequence
  Description of Artificial Sequence Synthetic primer
tttagggttatttaattatagggttagtta

SEQ ID NO: 117 - DNA, 25 bp, Artificial Sequence
  Description of Artificial Sequence Synthetic primer
tggggattgaggttggttattaatt

SEQ ID NO: 118 - DNA, 26 bp, Artificial Sequence
  Description of Artificial Sequence Synthetic primer
ggagattgggaggaataatttttttt

SEQ ID NO: 119 - DNA, 25 bp, Artificial Sequence
  Description of Artificial Sequence Synthetic primer
tgttttttaaattttttggagggat

SEQ ID NO: 120 - DNA, 22 bp, Artificial Sequence
  Description of Artificial Sequence Synthetic primer
gagttttagggtttgatgggaa

SEQ ID NO: 121 - DNA, 30 bp, Artificial Sequence
  Description of Artificial Sequence Synthetic primer
tggagtgggtaagatcattgcaagcatgac

SEQ ID NO: 122 - DNA, 27 bp, Artificial Sequence
  Description of Artificial Sequence Synthetic primer
gagggtattattttttgataggaagag

SEQ ID NO: 123 - DNA, 25 bp, Artificial Sequence
  Description of Artificial Sequence Synthetic primer
acaaagctgggttcctgctgggccc

SEQ ID NO: 124 - DNA, 29 bp, Artificial Sequence
  Description of Artificial Sequence Synthetic primer
ttagttttttaatttgttttgggggatat

SEQ ID NO: 125 - DNA, 28 bp, Artificial Sequence
  Description of Artificial Sequence Synthetic primer
ggtgtgtatttttagtttgtgtttggag

SEQ ID NO: 126 - DNA, 27 bp, Artificial Sequence
  Description of Artificial Sequence Synthetic primer
ggaagattttttaggttaagttggaga

SEQ ID NO: 127 - DNA, 26 bp, Artificial Sequence
  Description of Artificial Sequence Synthetic primer
ttgtttttttatggaaatgaaggatt

SEQ ID NO: 128 - DNA, 29 bp, Artificial Sequence
  Description of Artificial Sequence Synthetic primer
agtttaggttgatttagaataggattttg

SEQ ID NO: 129 - DNA, 25 bp, Artificial Sequence
  Description of Artificial Sequence Synthetic primer
cctgcccttggctgggtaatctctg

SEQ ID NO: 130 - DNA, 29 bp, Artificial Sequence
  Description of Artificial Sequence Synthetic primer
atggattttaggaatttgtttaaggttat

SEQ ID NO: 131 - DNA, 30 bp, Artificial Sequence
  Description of Artificial Sequence Synthetic primer
tttatttgtttttttggtagttatagagta

SEQ ID NO: 132 - DNA, 30 bp, Artificial Sequence
  Description of Artificial Sequence Synthetic primer
ttgttgatgttatatttttaggttttaatt

SEQ ID NO: 133 - DNA, 25 bp, Artificial Sequence
  Description of Artificial Sequence Synthetic primer
gtttgggattgttttggaggtatag

SEQ ID NO: 134 - DNA, 25 bp, Artificial Sequence
  Description of Artificial Sequence Synthetic primer
ttgtttgtttttgtagggttgttgg

SEQ ID NO: 135 - DNA, 22 bp, Artificial Sequence
  Description of Artificial Sequence Synthetic primer
gttgttttttggttgttttttt

SEQ ID NO: 136 - DNA, 27 bp, Artificial Sequence
  Description of Artificial Sequence Synthetic primer
tttaatttgtagtttgggggttgtttt

SEQ ID NO: 137 - DNA, 25 bp, Artificial Sequence
  Description of Artificial Sequence Synthetic primer
atttttttaggtaggtggtggggaa

SEQ ID NO: 138 - DNA, 25 bp, Artificial Sequence
  Description of Artificial Sequence Synthetic primer
gaggggaaagggttttatttttttt

SEQ ID NO: 139 - DNA, 26 bp, Artificial Sequence
  Description of Artificial Sequence Synthetic primer
ttttttttaggtttggagggtttttg

SEQ ID NO: 140 - DNA, 24 bp, Artificial Sequence
  Description of Artificial Sequence Synthetic primer
gggagagttggtttttatttattt

SEQ ID NO: 141 - DNA, 25 bp, Artificial Sequence
  Description of Artificial Sequence Synthetic primer
gtgagggttttgattttagaattaa

SEQ ID NO: 142 - DNA, 26 bp, Artificial Sequence
  Description of Artificial Sequence Synthetic primer
aaagaagttttgagaatgtttttttt

SEQ ID NO: 143 - DNA, 25 bp, Artificial Sequence
  Description of Artificial Sequence Synthetic primer
ggtttttaatttttttagggagggg

SEQ ID NO: 144 - DNA, 28 bp, Artificial Sequence
  Description of Artificial Sequence Synthetic primer
ctggtgacagccaggtaggtggaagttt

SEQ ID NO: 145 - DNA, 29 bp, Artificial Sequence
  Description of Artificial Sequence Synthetic primer
tatatggaggttttgttttgttttaaaaa

SEQ ID NO: 146 - DNA, 25 bp, Artificial Sequence
  Description of Artificial Sequence Synthetic primer
agggaagaagtgaccctggctgatg

SEQ ID NO: 147 - DNA, 25 bp, Artificial Sequence
  Description of Artificial Sequence Synthetic primer
gtgggagttgttggtttgaaataag

SEQ ID NO: 148 - DNA, 25 bp, Artificial Sequence
  Description of Artificial Sequence Synthetic primer
tttttttgggtagtaaagtgttggg

SEQ ID NO: 149 - DNA, 25 bp, Artificial Sequence
  Description of Artificial Sequence Synthetic primer
tttttttgggtagtaaagtgttggg

SEQ ID NO: 150 - DNA, 22 bp, Artificial Sequence
  Description of Artificial Sequence Synthetic primer
gctgtggggtggggcacacttg

SEQ ID NO: 151 - DNA, 25 bp, Artificial Sequence
  Description of Artificial Sequence Synthetic primer
tgggggtttagaggtatagtttttt

SEQ ID NO: 152 - DNA, 24 bp, Artificial Sequence
  Description of Artificial Sequence Synthetic primer
gggtttggggtttagtttgttttg

SEQ ID NO: 153 - DNA, 29 bp, Artificial Sequence
  Description of Artificial Sequence Synthetic primer
ttttttgttaggtaggttttagttattgt

SEQ ID NO: 154 - DNA, 30 bp, Artificial Sequence
  Description of Artificial Sequence Synthetic primer
tgatgtaaggatgtagggatttagagatta

SEQ ID NO: 155 - DNA, 22 bp, Artificial Sequence
  Description of Artificial Sequence Synthetic primer
tctggctgtgggggaccaggac

SEQ ID NO: 156 - DNA, 26 bp, Artificial Sequence
  Description of Artificial Sequence Synthetic primer
tgagtaatagggagggttttggattt

SEQ ID NO: 157 - DNA, 24 bp, Artificial Sequence
  Description of Artificial Sequence Synthetic primer
ggggatttttaggaattgtaggag

SEQ ID NO: 158 - DNA, 30 bp, Artificial Sequence
  Description of Artificial Sequence Synthetic primer
agatttttttaggaggttatagaaggtgtt

SEQ ID NO: 159 - DNA, 25 bp, Artificial Sequence
  Description of Artificial Sequence Synthetic primer
agtaagttaggagggtagtgggtgg

SEQ ID NO: 160 - DNA, 25 bp, Artificial Sequence
  Description of Artificial Sequence Synthetic primer
ttttttgggttgttttattttgttt

SEQ ID NO: 161 - DNA, 26 bp, Artificial Sequence
  Description of Artificial Sequence Synthetic primer
ctggggccctctgagagcaggcaggc

SEQ ID NO: 162 - DNA, 25 bp, Artificial Sequence
  Description of Artificial Sequence Synthetic primer
gtagagggggagttataggtgatgg

SEQ ID NO: 163 - DNA, 30 bp, Artificial Sequence
  Description of Artificial Sequence Synthetic primer
ggaggggagtttatttatttttttaatttt

SEQ ID NO: 164 - DNA, 28 bp, Artificial Sequence
  Description of Artificial Sequence Synthetic primer
tttttagaattttgtgtgtgtgtgtgta

SEQ ID NO: 165 - DNA, 30 bp, Artificial Sequence
  Description of Artificial Sequence Synthetic primer
aaattttgttgtattgagatattttaatgt

SEQ ID NO: 166 - DNA, 26 bp, Artificial Sequence
  Description of Artificial Sequence Synthetic primer
ggatggggaaactgaggctccaagca

SEQ ID NO: 167 - DNA, 29 bp, Artificial Sequence
  Description of Artificial Sequence Synthetic primer
tgtataaagtagaaatttaaatgttaggg

SEQ ID NO: 168 - DNA, 26 bp, Artificial Sequence
  Description of Artificial Sequence Synthetic primer
tttagggaaataaaatggaaatttta

SEQ ID NO: 169 - DNA, 25 bp, Artificial Sequence
  Description of Artificial Sequence Synthetic primer
gggtgttccctggcagagaggctct

SEQ ID NO: 170 - DNA, 29 bp, Artificial Sequence
  Description of Artificial Sequence Synthetic primer
taggattttgttgaatgaatgattgaatt

SEQ ID NO: 171 - DNA, 26 bp, Artificial Sequence
  Description of Artificial Sequence Synthetic primer
gattgttttggggtaataaaaagatt

SEQ ID NO: 172 - DNA, 22 bp, Artificial Sequence
  Description of Artificial Sequence Synthetic primer
gaattagggggaggggttgttt

SEQ ID NO: 173 - DNA, 30 bp, Artificial Sequence
  Description of Artificial Sequence Synthetic primer
agtttttttgtttttagtttggttttgtta

SEQ ID NO: 174 - DNA, 27 bp, Artificial Sequence
  Description of Artificial Sequence Synthetic primer
agtttgatttttattttggtgtagttt

SEQ ID NO: 175 - DNA, 26 bp, Artificial Sequence
  Description of Artificial Sequence Synthetic primer
tggtgttgtggttgatgtattttatg

SEQ ID NO: 176 - DNA, 25 bp, Artificial Sequence
  Description of Artificial Sequence Synthetic primer
gaggtggtgatgtttagggttagag

SEQ ID NO: 177 - DNA, 25 bp, Artificial Sequence
  Description of Artificial Sequence Synthetic primer
gggtaagtttggtatggtgttgttg

SEQ ID NO: 178 - DNA, 30 bp, Artificial Sequence
  Description of Artificial Sequence Synthetic primer
aagtttttgagaaatttttttaaaaattgt

SEQ ID NO: 179 - DNA, 24 bp, Artificial Sequence
  Description of Artificial Sequence Synthetic primer
gatggtgttaggtttttggtttgg

SEQ ID NO: 180 - DNA, 30 bp, Artificial Sequence
  Description of Artificial Sequence Synthetic primer
actaaccactttttcttttataactttcat

SEQ ID NO: 181 - DNA, 25 bp, Artificial Sequence
  Description of Artificial Sequence Synthetic primer
atcccataataactcccaactttac

SEQ ID NO: 182 - DNA, 25 bp, Artificial Sequence
  Description of Artificial Sequence Synthetic primer
aaaatccttatcccccataaacaac

SEQ ID NO: 183 - DNA, 25 bp, Artificial Sequence
  Description of Artificial Sequence Synthetic primer
tctaaactactccctccccaaatcc

SEQ ID NO: 184 - DNA, 25 bp, Artificial Sequence
  Description of Artificial Sequence Synthetic primer
tcctccctaaaacctccaaatttct

SEQ ID NO: 185 - DNA, 24 bp, Artificial Sequence
  Description of Artificial Sequence Synthetic primer
ctccccaaacaaccctacctctat

SEQ ID NO: 186 - DNA, 26 bp, Artificial Sequence
  Description of Artificial Sequence Synthetic primer
ctcaacctccattttctcctctaaac

SEQ ID NO: 187 - DNA, 25 bp, Artificial Sequence
  Description of Artificial Sequence Synthetic primer
ttccaacacccaaatctacttcctc

SEQ ID NO: 188 - DNA, 25 bp, Artificial Sequence
  Description of Artificial Sequence Synthetic primer
tccttaaaaaccaaaaactcctccc

SEQ ID NO: 189 - DNA, 24 bp, Artificial Sequence
  Description of Artificial Sequence Synthetic primer
aaaacaaacaactcccaacactac

SEQ ID NO: 190 - DNA, 25 bp, Artificial Sequence
  Description of Artificial Sequence Synthetic primer
ctccaaacaaaactacctccaactc

SEQ ID NO: 191 - DNA, 24 bp, Artificial Sequence
  Description of Artificial Sequence Synthetic primer
aaaactaccccaaacacacttccc

SEQ ID NO: 192 - DNA, 25 bp, Artificial Sequence
  Description of Artificial Sequence Synthetic primer
acaaaacaaaaacaccctcataacc

SEQ ID NO: 193 - DNA, 25 bp, Artificial Sequence
  Description of Artificial Sequence Synthetic primer
tctaatataaacccctaccccctcc

SEQ ID NO: 194 - DNA, 25 bp, Artificial Sequence
  Description of Artificial Sequence Synthetic primer
ataaacaacccacaccaaaacaacc

SEQ ID NO: 195 - DNA, 26 bp, Artificial Sequence
  Description of Artificial Sequence Synthetic primer
ccctttaaaccttttacaatcctaac

SEQ ID NO: 196 - DNA, 25 bp, Artificial Sequence
  Description of Artificial Sequence Synthetic primer
aaactaaaatccaccccaaaaaaac

SEQ ID NO: 197 - DNA, 26 bp, Artificial Sequence
  Description of Artificial Sequence Synthetic primer
ggctgtcacactggggctgctgctca

SEQ ID NO: 198 - DNA, 25 bp, Artificial Sequence
  Description of Artificial Sequence Synthetic primer
cttccaacctcaacaaaaaataacc

SEQ ID NO: 199 - DNA, 25 bp, Artificial Sequence
  Description of Artificial Sequence Synthetic primer
cataacacaacccaacttcaccaac

SEQ ID NO: 200 - DNA, 26 bp, Artificial Sequence
  Description of Artificial Sequence Synthetic primer
aaaccccaaacaactacacacctaac

SEQ ID NO: 201 - DNA, 25 bp, Artificial Sequence
  Description of Artificial Sequence Synthetic primer
aatcctacctctacttcctcccaac

SEQ ID NO: 202 - DNA, 25 bp, Artificial Sequence
  Description of Artificial Sequence Synthetic primer
cccctcccctcaacttaaaattaaa

SEQ ID NO: 203 - DNA, 25 bp, Artificial Sequence
  Description of Artificial Sequence Synthetic primer
aaaaatatatccctcccaaaaaccc

SEQ ID NO: 204 - DNA, 29 bp, Artificial Sequence
  Description of Artificial Sequence Synthetic primer
aatactttatctctacaacaaaactaccc

SEQ ID NO: 205 - DNA, 25 bp, Artificial Sequence
  Description of Artificial Sequence Synthetic primer
taccaaaacctaaaataccaacaac

SEQ ID NO: 206 - DNA, 25 bp, Artificial Sequence
  Description of Artificial Sequence Synthetic primer
ctaaaaactcccaaccctaaaaacc

SEQ ID NO: 207 - DNA, 24 bp, Artificial Sequence
  Description of Artificial Sequence Synthetic primer
gcctgcctgggcctgctggcagtg

SEQ ID NO: 208 - DNA, 30 bp, Artificial Sequence
  Description of Artificial Sequence Synthetic primer
aaaaaaaaccatactttccctataacacca

SEQ ID NO: 209 - DNA, 30 bp, Artificial Sequence
  Description of Artificial Sequence Synthetic primer
aaataaaaataaactaaacacaaaaaactc

SEQ ID NO: 210 - DNA, 25 bp, Artificial Sequence
  Description of Artificial Sequence Synthetic primer
aatcctaactcccaaaaacccactt

SEQ ID NO: 211 - DNA, 28 bp, Artificial Sequence
  Description of Artificial Sequence Synthetic primer
gtgaccctgggaaatgcttctatccctg

SEQ ID NO: 212 - DNA, 25 bp, Artificial Sequence
  Description of Artificial Sequence Synthetic primer
cactcaaaaaaccccaaaacctaac

SEQ ID NO: 213 - DNA, 26 bp, Artificial Sequence
  Description of Artificial Sequence Synthetic primer
ctacaaactacacaaccctccaactc

SEQ ID NO: 214 - DNA, 25 bp, Artificial Sequence
  Description of Artificial Sequence Synthetic primer
acctataactaaaaaacccccaaac

SEQ ID NO: 215 - DNA, 25 bp, Artificial Sequence
  Description of Artificial Sequence Synthetic primer
aggaggagggaagggagtccacccc

SEQ ID NO: 216 - DNA, 24 bp, Artificial Sequence
  Description of Artificial Sequence Synthetic primer
acctttacccccaatacctacctc

SEQ ID NO: 217 - DNA, 26 bp, Artificial Sequence
  Description of Artificial Sequence Synthetic primer
ccaaaaactaaccccactacatcaac

SEQ ID NO: 218 - DNA, 26 bp, Artificial Sequence
  Description of Artificial Sequence Synthetic primer
tcaatctccaatccttttaaaaaaaa

SEQ ID NO: 219 - DNA, 22 bp, Artificial Sequence
  Description of Artificial Sequence Synthetic primer
accaatccctataaccccctcc

SEQ ID NO: 220 - DNA, 25 bp, Artificial Sequence
  Description of Artificial Sequence Synthetic primer
ccaaaaaccacaaacaaccttaaac

SEQ ID NO: 221 - DNA, 25 bp, Artificial Sequence
  Description of Artificial Sequence Synthetic primer
aaacaacaaaaaaaccacctaaatc

SEQ ID NO: 222 - DNA, 25 bp, Artificial Sequence
  Description of Artificial Sequence Synthetic primer
ttactcctccaaataaacccaatcc

SEQ ID NO: 223 - DNA, 24 bp, Artificial Sequence
  Description of Artificial Sequence Synthetic primer
tcaaaaccaaaataacaaaactcc

SEQ ID NO: 224 - DNA, 25 bp, Artificial Sequence
  Description of Artificial Sequence Synthetic primer
taacccaaaaatacaaattttcaac

SEQ ID NO: 225 - DNA, 25 bp, Artificial Sequence
  Description of Artificial Sequence Synthetic primer
aaaaaaattcccactttaaaaaaac

SEQ ID NO: 226 - DNA, 24 bp, Artificial Sequence
  Description of Artificial Sequence Synthetic primer
cccacctactaaataaaacccaac

SEQ ID NO: 227 - DNA, 26 bp, Artificial Sequence
  Description of Artificial Sequence Synthetic primer
tccaaataataaaacacctactaacc

SEQ ID NO: 228 - DNA, 30 bp, Artificial Sequence
  Description of Artificial Sequence Synthetic primer
aatctaatacaataaaaccatcccaaatac

SEQ ID NO: 229 - DNA, 25 bp, Artificial Sequence
  Description of Artificial Sequence Synthetic primer
acctatacacccaacctacacaccc

SEQ ID NO: 230 - DNA, 25 bp, Artificial Sequence
  Description of Artificial Sequence Synthetic primer
aaaaaactcctcactttaaaaaaaa

SEQ ID NO: 231 - DNA, 25 bp, Artificial Sequence
  Description of Artificial Sequence Synthetic primer
aaaaaacaatcttcaaaaacccacc

SEQ ID NO: 232 - DNA, 30 bp, Artificial Sequence
  Description of Artificial Sequence Synthetic primer
aaacactattatcccccatttacaaataaa

SEQ ID NO: 233 - DNA, 26 bp, Artificial Sequence
  Description of Artificial Sequence Synthetic primer
aataaaaccttcctttaatcccctcc

SEQ ID NO: 234 - DNA, 26 bp, Artificial Sequence
  Description of Artificial Sequence Synthetic primer
ccctcttcctcccctactaatcctac

SEQ ID NO: 235 - DNA, 25 bp, Artificial Sequence
  Description of Artificial Sequence Synthetic primer
ccaaaccaataaaaaatctcccaac

SEQ ID NO: 236 - DNA, 23 bp, Artificial Sequence
  Description of Artificial Sequence Synthetic primer
aaaacccataaaaaccacaaccc

SEQ ID NO: 237 - DNA, 26 bp, Artificial Sequence
  Description of Artificial Sequence Synthetic primer
aaaactaacattttcaacaaaaactc

SEQ ID NO: 238 - DNA, 27 bp, Artificial Sequence
  Description of Artificial Sequence Synthetic primer
aaaaccctacctatttttcttaatccc

SEQ ID NO: 239 - DNA, 25 bp, Artificial Sequence
  Description of Artificial Sequence Synthetic primer
tttaaaaaccacctaaccccaaatc

SEQ ID NO: 240 - DNA, 25 bp, Artificial Sequence
  Description of Artificial Sequence Synthetic primer
ccccaaaactttaatcctatctccc

SEQ ID NO: 241 - DNA, 26 bp, Artificial Sequence
  Description of Artificial Sequence Synthetic primer
ggccataactagggaggaaggagggc

SEQ ID NO: 242 - DNA, 25 bp, Artificial Sequence
  Description of Artificial Sequence Synthetic primer
aaacaaattcaaccccaaattcaac

SEQ ID NO: 243 - DNA, 24 bp, Artificial Sequence
  Description of Artificial Sequence Synthetic primer
actcttccaaaccttaaaaacccc

SEQ ID NO: 244 - DNA, 25 bp, Artificial Sequence
  Description of Artificial Sequence Synthetic primer
ccaacccaacccaacaataataaaa

SEQ ID NO: 245 - DNA, 25 bp, Artificial Sequence
  Description of Artificial Sequence Synthetic primer
taatctccctccaaaaattccaaca

SEQ ID NO: 246 - DNA, 27 bp, Artificial Sequence
  Description of Artificial Sequence Synthetic primer
cccatactaaaaactctaaaccccatc

SEQ ID NO: 247 - DNA, 26 bp, Artificial Sequence
  Description of Artificial Sequence Synthetic primer
acccctcacaccattatcactatcaa

SEQ ID NO: 248 - DNA, 26 bp, Artificial Sequence
  Description of Artificial Sequence Synthetic primer
aaaacaattctaaccccacacatttc

SEQ ID NO: 249 - DNA, 25 bp, Artificial Sequence
  Description of Artificial Sequence Synthetic primer
ctaacaaaactccaaaccaatcacc

SEQ ID NO: 250 - DNA, 30 bp, Artificial Sequence
  Description of Artificial Sequence Synthetic primer
aaaaaacaaacatcttctctttccctacta

SEQ ID NO: 251 - DNA, 25 bp, Artificial Sequence
  Description of Artificial Sequence Synthetic primer
tcaaacaaaaaaccaattccaaatc

SEQ ID NO: 252 - DNA, 25 bp, Artificial Sequence
  Description of Artificial Sequence Synthetic primer
aaaaacccaaaaccctaatccctac

SEQ ID NO: 253 - DNA, 24 bp, Artificial Sequence
  Description of Artificial Sequence Synthetic primer
aacaaaccaccaaacaaacacatc

SEQ ID NO: 254 - DNA, 30 bp, Artificial Sequence
  Description of Artificial Sequence Synthetic primer
aaccactttttcttttataactttcatatc

SEQ ID NO: 255 - DNA, 22 bp, Artificial Sequence
  Description of Artificial Sequence Synthetic primer
ctacaacaaccccaactccctc

SEQ ID NO: 256 - DNA, 25 bp, Artificial Sequence
  Description of Artificial Sequence Synthetic primer
aatccaaaactcactaacaaaaatc

SEQ ID NO: 257 - DNA, 26 bp, Artificial Sequence
  Description of Artificial Sequence Synthetic primer
ccactaaaaccctaaacaactactac

SEQ ID NO: 258 - DNA, 30 bp, Artificial Sequence
  Description of Artificial Sequence Synthetic primer
acaaaaacaaaactaaatttaatcttttaa

SEQ ID NO: 259 - DNA, 25 bp, Artificial Sequence
  Description of Artificial Sequence Synthetic primer
caaatcaaaatctaatttcaaaacc

SEQ ID NO: 260 - DNA, 25 bp, Artificial Sequence
  Description of Artificial Sequence Synthetic primer
caaatcaaaatctaatttcaaaacc

SEQ ID NO: 261 - DNA, 26 bp, Artificial Sequence
  Description of Artificial Sequence Synthetic primer
ccccacccacaaaaaaataaataaaa

SEQ ID NO: 262 - DNA, 30 bp, Artificial Sequence
  Description of Artificial Sequence Synthetic primer
attacacaaaaaacttaaaccaaaatcaac

SEQ ID NO: 263 - DNA, 25 bp, Artificial Sequence
  Description of Artificial Sequence Synthetic primer
accctctctccctctataaacctcc

SEQ ID NO: 264 - DNA, 25 bp, Artificial Sequence
  Description of Artificial Sequence Synthetic primer
taaactcactccccaacataaaaac

SEQ ID NO: 265 - DNA, 30 bp, Artificial Sequence
  Description of Artificial Sequence Synthetic primer
aaacaaccaatcaaataactaaatttacca

SEQ ID NO: 266 - DNA, 25 bp, Artificial Sequence
  Description of Artificial Sequence Synthetic primer
aaaaatctctcaaaaaccaatcaac

SEQ ID NO: 267 - DNA, 24 bp, Artificial Sequence
  Description of Artificial Sequence Synthetic primer
atcctaaatctcacctaaaacccc

SEQ ID NO: 268 - DNA, 25 bp, Artificial Sequence
  Description of Artificial Sequence Synthetic primer
acccccaactactatccctcactac

SEQ ID NO: 269 - DNA, 25 bp, Artificial Sequence
  Description of Artificial Sequence Synthetic primer
aaaaaaaacctcctcccacaaaaaa

SEQ ID NO: 270 - DNA, 25 bp, Artificial Sequence
  Description of Artificial Sequence Synthetic primer
aaaatccttatcccccataaacaac

SEQ ID NO: 271 - DNA, 25 bp, Artificial Sequence
  Description of Artificial Sequence Synthetic primer
caaattcctcaaaactcaaatatcc

SEQ ID NO: 272 - DNA, 30 bp, Artificial Sequence
  Description of Artificial Sequence Synthetic primer
atacaactcaaaaaacaatacctcattcat

SEQ ID NO: 273 - DNA, 23 bp, Artificial Sequence
  Description of Artificial Sequence Synthetic primer
aaaaacttcaaccaccaaaaaac

SEQ ID NO: 274 - DNA, 25 bp, Artificial Sequence
  Description of Artificial Sequence Synthetic primer
acaacctaacaccccactttaccat

SEQ ID NO: 275 - DNA, 25 bp, Artificial Sequence
  Description of Artificial Sequence Synthetic primer
aacccaaaaaatctctccaattacc

SEQ ID NO: 276 - DNA, 28 bp, Artificial Sequence
  Description of Artificial Sequence Synthetic primer
tccccctcaaaaaaatttaattcataaa

SEQ ID NO: 277 - DNA, 22 bp, Artificial Sequence
  Description of Artificial Sequence Synthetic primer
cccattccaactacctaacccc

SEQ ID NO: 278 - DNA, 25 bp, Artificial Sequence
  Description of Artificial Sequence Synthetic primer
aaaaaaacaaactacctttcctccc

SEQ ID NO: 279 - DNA, 25 bp, Artificial Sequence
  Description of Artificial Sequence Synthetic primer
caaaacctctcccaaaatctcaaac

SEQ ID NO: 280 - DNA, 23 bp, Artificial Sequence
  Description of Artificial Sequence Synthetic primer
gcaggggtggaactggattctgc

SEQ ID NO: 281 - DNA, 25 bp, Artificial Sequence
  Description of Artificial Sequence Synthetic primer
aaaccaaaccaataaccaaaaaatc

SEQ ID NO: 282 - DNA, 25 bp, Artificial Sequence
  Description of Artificial Sequence Synthetic primer
aatctaaactcccccacctcctaac

SEQ ID NO: 283 - DNA, 28 bp, Artificial Sequence
  Description of Artificial Sequence Synthetic primer
aaaaataacctccttaccaatcaaaacc

SEQ ID NO: 284 - DNA, 29 bp, Artificial Sequence
  Description of Artificial Sequence Synthetic primer
aacttccttcaatcatccaatctttattc

SEQ ID NO: 285 - DNA, 25 bp, Artificial Sequence
  Description of Artificial Sequence Synthetic primer
ccttttcctatcacaaaaataatcc

SEQ ID NO: 286 - DNA, 25 bp, Artificial Sequence
  Description of Artificial Sequence Synthetic primer
ggaaggctgaactgctgagtctgac

SEQ ID NO: 287 - DNA, 25 bp, Artificial Sequence
  Description of Artificial Sequence Synthetic primer
ccccaatccaacctaaactctaaac

SEQ ID NO: 288 - DNA, 29 bp, Artificial Sequence
  Description of Artificial Sequence Synthetic primer
aaatcacacaaacctcctcattaactact

SEQ ID NO: 289 - DNA, 25 bp, Artificial Sequence
  Description of Artificial Sequence Synthetic primer
aaaacttcctcacccctaacttctc

SEQ ID NO: 290 - DNA, 27 bp, Artificial Sequence
  Description of Artificial Sequence Synthetic primer
gaggtagaatggatccccttggccttc

SEQ ID NO: 291 - DNA, 25 bp, Artificial Sequence
  Description of Artificial Sequence Synthetic primer
caaaataactccctccaaacaaaac

SEQ ID NO: 292 - DNA, 29 bp, Artificial Sequence
  Description of Artificial Sequence Synthetic primer
aatatattctcccatctatctcactcaaa

SEQ ID NO: 293 - DNA, 26 bp, Artificial Sequence
  Description of Artificial Sequence Synthetic primer
cctctcaactactatcaacctcctcc

SEQ ID NO: 294 - DNA, 26 bp, Artificial Sequence
  Description of Artificial Sequence Synthetic primer
ggaggaggttgacagtagctgagagg

SEQ ID NO: 295 - DNA, 25 bp, Artificial Sequence
  Description of Artificial Sequence Synthetic primer
aaaaaaactaaaccaccaaaaaccc

SEQ ID NO: 296 - DNA, 25 bp, Artificial Sequence
  Description of Artificial Sequence Synthetic primer
ctcccaaattctctaaaccccaact

SEQ ID NO: 297 - DNA, 25 bp, Artificial Sequence
  Description of Artificial Sequence Synthetic primer
ctccctaacacctaaactcccaaac

SEQ ID NO: 298 - DNA, 24 bp, Artificial Sequence
  Description of Artificial Sequence Synthetic primer
aaaaacccaatcctccttccttac

SEQ ID NO: 299 - DNA, 30 bp, Artificial Sequence
  Description of Artificial Sequence Synthetic primer
ccaattcttttaaaaacactatattcctta

SEQ ID NO: 300 - DNA, 27 bp, Artificial Sequence
  Description of Artificial Sequence Synthetic primer
taggtctgcagagtggtcttcctggta

SEQ ID NO: 301 - DNA, 28 bp, Artificial Sequence
  Description of Artificial Sequence Synthetic primer
caaactaccaataccactcactcactac

SEQ ID NO: 302 - DNA, 27 bp, Artificial Sequence
  Description of Artificial Sequence Synthetic primer
ggagcagcacccttccaggggaggtgg

SEQ ID NO: 303 - DNA, 29 bp, Artificial Sequence
  Description of Artificial Sequence Synthetic primer
tttatcaaaacatcattttctccctataa

SEQ ID NO: 304 - DNA, 26 bp, Artificial Sequence
  Description of Artificial Sequence Synthetic primer
aatacccttctacccacatcccatat

SEQ ID NO: 305 - DNA, 25 bp, Artificial Sequence
  Description of Artificial Sequence Synthetic primer
aaaaaccactaccttaactcccctc

SEQ ID NO: 306 - DNA, 24 bp, Artificial Sequence
  Description of Artificial Sequence Synthetic primer
aacaaaccccctctccctactacc

SEQ ID NO: 307 - DNA, 26 bp, Artificial Sequence
  Description of Artificial Sequence Synthetic primer
cccctaaaacaaaaataataaccaac

SEQ ID NO: 308 - DNA, 24 bp, Artificial Sequence
  Description of Artificial Sequence Synthetic primer
gtctggggccagcagggggcacta

SEQ ID NO: 309 - DNA, 25 bp, Artificial Sequence
  Description of Artificial Sequence Synthetic primer
ataaaaaatctaccccaaccccttc

SEQ ID NO: 310 - DNA, 23 bp, Artificial Sequence
  Description of Artificial Sequence Synthetic primer
cacaatccaaacaaaaaaccctc

SEQ ID NO: 311 - DNA, 22 bp, Artificial Sequence
  Description of Artificial Sequence Synthetic primer
ctaaaaaaccccacaccccaac

SEQ ID NO: 312 - DNA, 30 bp, Artificial Sequence
  Description of Artificial Sequence Synthetic primer
aaaaaaacaaacaaataacctacctctcac

SEQ ID NO: 313 - DNA, 25 bp, Artificial Sequence
  Description of Artificial Sequence Synthetic primer
aattcccaaaaaaatcccaaattct

SEQ ID NO: 314 - DNA, 25 bp, Artificial Sequence
  Description of Artificial Sequence Synthetic primer
atccctacacccaaatttccattac

SEQ ID NO: 315 - DNA, 25 bp, Artificial Sequence
  Description of Artificial Sequence Synthetic primer
taaccaactaactccaatcactccc

SEQ ID NO: 316 - DNA, 25 bp, Artificial Sequence
  Description of Artificial Sequence Synthetic primer
cctcaaaacccaacaaactcaaact

SEQ ID NO: 317 - DNA, 25 bp, Artificial Sequence
  Description of Artificial Sequence Synthetic primer
aacctacttcataaccctaatcatc

SEQ ID NO: 318 - DNA, 26 bp, Artificial Sequence
  Description of Artificial Sequence Synthetic primer
aactacaaaaaattttcccacttccc

SEQ ID NO: 319 - DNA, 25 bp, Artificial Sequence
  Description of Artificial Sequence Synthetic primer
acccacaaaaatccctcattctcta

SEQ ID NO: 320 - DNA, 25 bp, Artificial Sequence
  Description of Artificial Sequence Synthetic primer
ctacctccacctcactcttaataac

SEQ ID NO: 321 - DNA, 30 bp, Artificial Sequence
  Description of Artificial Sequence Synthetic primer
ttccaaacacactttatataaaatctacaa

SEQ ID NO: 322 - DNA, 25 bp, Artificial Sequence
  Description of Artificial Sequence Synthetic primer
cccaccaataactcctcctcctact

SEQ ID NO: 323 - DNA, 25 bp, Artificial Sequence
  Description of Artificial Sequence Synthetic primer
ccactttgggtctagggagaggagg

SEQ ID NO: 324 - DNA, 26 bp, Artificial Sequence
  Description of Artificial Sequence Synthetic primer
aaataaaccaacccttacccaatctc

SEQ ID NO: 325 - DNA, 25 bp, Artificial Sequence
  Description of Artificial Sequence Synthetic primer
gggtggatcacctgaggtcaggagt

SEQ ID NO: 326 - DNA, 29 bp, Artificial Sequence
  Description of Artificial Sequence Synthetic primer
ccctaactttatctctctataaatacacc

SEQ ID NO: 327 - DNA, 30 bp, Artificial Sequence
  Description of Artificial Sequence Synthetic primer
aaaactcaaaaaacttatctttaaaacaca

SEQ ID NO: 328 - DNA, 30 bp, Artificial Sequence; identical to SEQ ID NO: 327
  Description of Artificial Sequence Synthetic primer

SEQ ID NO: 329 - DNA, 30 bp, Artificial Sequence
  Description of Artificial Sequence Synthetic primer
ttacatggaggacctgcaggagctcaccat

SEQ ID NO: 330 - DNA, 25 bp, Artificial Sequence
  Description of Artificial Sequence Synthetic primer
ccacacctatctaaacaccaaaatc

SEQ ID NO: 331 - DNA, 25 bp, Artificial Sequence
  Description of Artificial Sequence Synthetic primer
taaaaactccaatccaactttccac

SEQ ID NO: 332 - DNA, 24 bp, Artificial Sequence
  Description of Artificial Sequence Synthetic primer
ccaaactaccaaatccccctactc

SEQ ID NO: 333 - DNA, 25 bp, Artificial Sequence
  Description of Artificial Sequence Synthetic primer
tcaaaccaaccctaatacactaccc

SEQ ID NO: 334 - DNA, 27 bp, Artificial Sequence
  Description of Artificial Sequence Synthetic primer
aaagtgggctccactaagctgggaagg

SEQ ID NO: 335 - DNA, 30 bp, Artificial Sequence
  Description of Artificial Sequence Synthetic primer
aactaaaataactaacaacccaaataaata

SEQ ID NO: 336 - DNA, 27 bp, Artificial Sequence
  Description of Artificial Sequence Synthetic primer
ccatacccaaaaaaaactaactaaacc

SEQ ID NO: 337 - DNA, 25 bp, Artificial Sequence
  Description of Artificial Sequence Synthetic primer
tccaaatccaaaactcccaatctac

SEQ ID NO: 338 - DNA, 25 bp, Artificial Sequence
  Description of Artificial Sequence Synthetic primer
tatcaccccaaaaaaactatctccc

SEQ ID NO: 339 - DNA, 25 bp, Artificial Sequence
  Description of Artificial Sequence Synthetic primer
aacaacaaaatcttctttccccatc

SEQ ID NO: 340 - DNA, 25 bp, Artificial Sequence
  Description of Artificial Sequence Synthetic primer
ccagtggatgggcctggtttgttcc

SEQ ID NO: 341 - DNA, 24 bp, Artificial Sequence
  Description of Artificial Sequence Synthetic primer
cccccaaccaaaactaaaaaaaac

SEQ ID NO: 342 - DNA, 27 bp, Artificial Sequence
  Description of Artificial Sequence Synthetic primer
acctcttaatcccctccctattatacc

SEQ ID NO: 343 - DNA, 25 bp, Artificial Sequence
  Description of Artificial Sequence Synthetic primer
aacaaaacatcctatccaaacatcc

SEQ ID NO: 344 - DNA, 25 bp, Artificial Sequence
  Description of Artificial Sequence Synthetic primer
aaaactaataccaaacaaaaacccc

SEQ ID NO: 345 - DNA, 25 bp, Artificial Sequence
  Description of Artificial Sequence Synthetic primer
gacctgggaggccacccattgccca

SEQ ID NO: 346 - DNA, 25 bp, Artificial Sequence
  Description of Artificial Sequence Synthetic primer
cacaaatttaatctccattctcctc

SEQ ID NO: 347 - DNA, 25 bp, Artificial Sequence
  Description of Artificial Sequence Synthetic primer
cataaaaatcaataaataaccccac

SEQ ID NO: 348 - DNA, 30 bp, Artificial Sequence
  Description of Artificial Sequence Synthetic primer
gcccaagaagattgtaaatgccaagaaagg

SEQ ID NO: 349 - DNA, 25 bp, Artificial Sequence
  Description of Artificial Sequence Synthetic primer
tttaaaaaccacctaaccccaaatc

SEQ ID NO: 350 - DNA, 25 bp, Artificial Sequence
  Description of Artificial Sequence Synthetic primer
taatctccctccaaaaattccaaca

SEQ ID NO: 351 - DNA, 24 bp, Artificial Sequence
  Description of Artificial Sequence Synthetic primer
aaaccatcttcctcccctacaaaa

SEQ ID NO: 352 - DNA, 24 bp, Artificial Sequence
  Description of Artificial Sequence Synthetic primer
aaaaaaaatccctacaccacctcc

SEQ ID NO: 353 - DNA, 27 bp, Artificial Sequence
  Description of Artificial Sequence Synthetic primer
aatacaaaaaacacaacccctacaacc

SEQ ID NO: 354 - DNA, 28 bp, Artificial Sequence
  Description of Artificial Sequence Synthetic primer
caatctcctttaacctaactaaacaatc

SEQ ID NO: 355 - DNA, 25 bp, Artificial Sequence
  Description of Artificial Sequence Synthetic primer
tccaaattttaacaactccaaaacc

SEQ ID NO: 356 - DNA, 24 bp, Artificial Sequence
  Description of Artificial Sequence Synthetic primer
acttaaccttcctactccccctcc

SEQ ID NO: 357 - DNA, 25 bp, Artificial Sequence
  Description of Artificial Sequence Synthetic primer
aaacaaacaacctccccacttacat

SEQ ID NO: 358 - DNA, 26 bp, Artificial Sequence
  Description of Artificial Sequence Synthetic primer
cctaaattctccctaaacccctccta

SEQ ID NO: 359 - DNA, 26 bp, Artificial Sequence
  Description of Artificial Sequence Synthetic primer
cagtaatacgactcactatagggaga

SEQ ID NO: 360 - DNA, 54 bp, Artificial Sequence
  Description of Artificial Sequence Synthetic primer
cagtaatacgactcactatagggagaaggctgttagtttttattttatttttaa

SEQ ID NO: 361 - DNA, 33 bp, Artificial Sequence
  Description of Artificial Sequence Synthetic primer
aggaagagagaaccactatctcccctcaaaaaa

SEQ ID NO: 362 - DNA, 34 bp, Artificial Sequence
  Description of Artificial Sequence Synthetic primer
aggaagagaggttagtttttattttatttttaat

SEQ ID NO: 363 - DNA, 54 bp, Artificial Sequence
  Description of Artificial Sequence Synthetic primer
cagtaatacgactcactatagggagaaggctaaccactatctcccctcaaaaaa

SEQ ID NO: 364 - DNA, 416 bp, Artificial Sequence
  Description of Artificial Sequence Synthetic primer
gggtttgggagagtttgtgaggtcgtttatcgtttgttagtagagtgcgttcgcgagtcgtaagtatagttcggtaatatgcggtttttagataggaaagtggtcgcgaatgggatcggggtgtttagcggttgtggggattttgttttgcggaaatcgcggtgacgagtataagttcggttaattggatgggaatcggtttggggggttggtatcgcgtttattagggggtttgcggtatttttttttgttttttagtattttatttttattttttaggaacgtgaggtttgagtcgtgatggtggtaggaaggggttttttgtgttattcgagtttttagggattcgtagttggtttttagttatgtgtaaagtatgtgtagggcgttggtaggtagggagtagtaggtatggt

SEQ ID NO: 365 - DNA, 416 bp, Artificial Sequence
  Description of Artificial Sequence Synthetic primer
gggtttgggagagtttgtgaggttgtttattgtttgttagtagagtgtgtttgtgagttgtaagtatagtttggtaatatgtggtttttagataggaaagtggttgtgaatgggattggggtgtttagtggttgtggggattttgttttgtggaaattgtggtgatgagtataagtttggttaattggatgggaattggtttggggggttggtattgtgtttattagggggtttgtggtatttttttttgttttttagtattttatttttattttttaggaatgtgaggtttgagttgtgatggtggtaggaaggggttttttgtgttatttgagtttttagggatttgtagttggtttttagttatgtgtaaagtatgtgtagggtgttggtaggtagggagtagtaggtatggt

SEQ ID NO: 366 - DNA, 10 bp, Artificial Sequence
  Description of Artificial Sequence Synthetic primer
cgcaaccact

SEQ ID NO: 367 - DNA, 10 bp, Artificial Sequence
  Description of Artificial Sequence Synthetic primer
cgcgaccact

SEQ ID NO: 368 - DNA, 10 bp, Artificial Sequence
  Description of Artificial Sequence Synthetic primer
cacaaccact

SEQ ID NO: 369 - DNA, 55 bp, Artificial Sequence
  Description of Artificial Sequence Synthetic primer
cagtaatacgactcactatagggagaaggctgttagtttttattttatttttaat

SEQ ID NO: 370 - DNA, 10 bp, Artificial Sequence
  Description of Artificial Sequence Synthetic primer
ggtaatatgc

SEQ ID NO: 371 - DNA, 10 bp, Artificial Sequence
  Description of Artificial Sequence Synthetic primer
gaatgggatc

SEQ ID NO: 372 - DNA, 10 bp, Artificial Sequence
  Description of Artificial Sequence Synthetic primer
gggagaaggc

SEQ ID NO: 373 - DNA, 12 bp, Artificial Sequence
  Description of Artificial Sequence Synthetic primer
gagtataagttc

SEQ ID NO: 374 - DNA, 12 bp, Artificial Sequence
  Description of Artificial Sequence Synthetic primer
ggggtgtttagc

SEQ ID NO: 375 - DNA, 13 bp, Artificial Sequence
  Description of Artificial Sequence Synthetic primer
gtaagtatagttc

SEQ ID NO: 376 - DNA, 14 bp, Artificial Sequence
  Description of Artificial Sequence Synthetic primer
gtgaggtttgagtc

SEQ ID NO: 377 - DNA, 16 bp, Artificial Sequence
  Description of Artificial Sequence Synthetic primer
gagtttttagggattc

SEQ ID NO: 378 - DNA, 17 bp, Artificial Sequence
  Description of Artificial Sequence Synthetic primer
gtttgttagtagagtgc

SEQ ID NO: 379 - DNA, 18 bp, Artificial Sequence
  Description of Artificial Sequence Synthetic primer
gtttattagggggtttgc

SEQ ID NO: 380 - DNA, 19 bp, Artificial Sequence
  Description of Artificial Sequence Synthetic primer
ggttaattggatgggaatc

SEQ ID NO: 381 - DNA, 19 bp, Artificial Sequence
  Description of Artificial Sequence Synthetic primer
ggtttggggggttggtatc

SEQ ID NO: 382 - DNA, 22 bp, Artificial Sequence
  Description of Artificial Sequence Synthetic primer
ggttgtggggattttgttttgc

SEQ ID NO: 383 - DNA, 23 bp, Artificial Sequence
  Description of Artificial Sequence Synthetic primer
ggtttttagataggaaagtggtc

SEQ ID NO: 384 - DNA, 25 bp, Artificial Sequence
  Description of Artificial Sequence Synthetic primer
tgggtttgggagagtttgtgaggtc

SEQ ID NO: 385 - DNA, 30 bp, Artificial Sequence
  Description of Artificial Sequence Synthetic primer
gttggtaggtagggagtagtaggtatggtc

SEQ ID NO: 386 - DNA, 35 bp, Artificial Sequence
  Description of Artificial Sequence Synthetic primer
gtgatggtggtaggaaggggttttttgtgttattc

SEQ ID NO: 387 - DNA, 39 bp, Artificial Sequence
  Description of Artificial Sequence Synthetic primer
gtagttggtttttagttatgtgtaaagtatgtgtagggc

SEQ ID NO: 388 - DNA, 47 bp, Artificial Sequence
  Description of Artificial Sequence Synthetic primer
ggtatttttttttgttttttagtattttatttttattttttaggaac

SEQ ID NO: 389 - DNA, 10 bp, Artificial Sequence
  Description of Artificial Sequence Synthetic primer
gggagaaggc

SEQ ID NO: 390 - DNA, 10 bp, Artificial Sequence
  Description of Artificial Sequence Synthetic primer
gggagaaggc

SEQ ID NO: 391 - DNA, 420 bp, Artificial Sequence
  Description of Artificial Sequence Synthetic primer
tgggtttgggagagtttgtgaggttgtttattgtttgttagtagagtgtgtttgtgagttgtaagtatagtttggtaatatgtggtttttagataggaaagtggttgtgaatgggattggggtgtttagtggttgtggggattttgttttgtggaaattgtggtgatgagtataagtttggttaattggatgggaattggtttggggggttggtattgtgtttattagggggtttgtggtatttttttttgttttttagtattttatttttattttttaggaatgtgaggtttgagttgtgatggtggtaggaaggggttttttgtgttatttgagtttttagggatttgtagttggtttttagttatgtgtaaagtatgtgtagggtgttggtaggtagggagtagtaggtatggtagc

SEQ ID NO: 392 - DNA, 433 bp, Homo sapiens
ggagcaggcgggtcggtggtactcgccgtcggcgcccaaagcggcggacgccgggtacggctgctgattggcattataagcgaacccgttggctgcctggtaggggtagccaccgtagatcgccgagctgtcgtagtaggtcgctttttgcatcgcgttgtttcacgatcttgatcgcacactctgacaggggtttgacacccgtgagggcgcacattggcacgcccccgcggtcacgtgacactccgccgccaatggccgccccgcgcagacctggtggggcgagaagcgcagcgcggtgagggctccgcgcaaatccatcttactctcaatagctaagtgacatgaaagccataaaagaaaaagtggtcagcaatatttagcagcacgacttggccccgggcgcagggagccgtgctataaaaaaccgctg

SEQ ID NO: 393 - DNA, 730 bp, Homo sapiens
agaaaggtgatacagaattggagaggtcggagtttttgtattaactgtattaaatgcgaatcccgagaaaatttcccttaactacgtcctgtagttatatggatatgaagacttatgtgaactttgaaagacgtgtctacataagttgaaatgtccccaatgattcagctgatgcgcgtttctctacttgccctttctagagaggtgcaacggaagccagaacattcctcctggaaattcaacctgtttcgcagtttctcgaggaatcagcattcagtcaatccgggccgggagcagtcatctgtggtgaggctgattggctgggcaggaacagcgccggggcgtgggctgagcacagccgcttcgctctctttgccacaggaagcctgagctcattcgagtagcggctcttccaagctcaaagaagcagaggccgctgttcgtttcctttaggtctttccactaaagtcggagtatcttcttccaaaatttcacgtcttggtggccgttccaaggagcgcgaggtaggggcacgcaaagctgggagctactatgggacagttcccaagtgtcaggctttcagatttcctgaacttggtcttcacgggagaagggcttcttgaggcgtggatagtgtgaagtcctctggcaagtccatggggaccaagtggggttagatctagactcaggagctcctggagcagcgcccaaaccgtagtggcactggacc

SEQ ID NO: 394 - DNA, 1405 bp, Homo sapiens
gcaggtggaggcagctttgtgggcccagctggggctgactctgctgggcttttgccctcaggtaaagccgaactcctaacctcaggtgccaaatcccccacgggggcctccgaccacttcctgggccgccgtggcagccccttgctgagctggtccgcggtggcgcagaccaagcggaaggcggtggcagcggccagcaaggggccgggggtgctgcagaacctcttccagctcaacggcagcagcaagaagctgcgggcccgcgaggccctgttccccgtgcacagcgtggccacacccatatttggcaacggcttccgcgccgactccttcagcagcctggccagctcctacgcgcccttcgtcggggggaccgggccgggcctccccaggggagcccacaagctgctgcgggctaagaaggccgagagggtggaggccgagaagggtgggcggcggcgggcgggcggtgagttcctggtcaagctggaccacgagggtgtgacctcccccaagaacaagacctgcaaggcgttgctcatgggggacaaggacttcagccccaagctcgggcggcccctgcccagccccagctatgtgcacccggcccttgtgggcaaggacaagaaggggcgggcacccatccccccgctgcccatggggctggcgctgcgcaagtacgcgggccaggcagagttcccgctgccctacgacagcgactgccacagctccttctcggacgaggacgaggacgggccggggctggcggccggcgtgccctcccgcttcctcgcccgcctgtccgtgtcctcttcctcctctggctcgtccacctcctcctcctcaggctccgtgtccacctccagcctctgctcctccgacaacgaggactcgtcctacagctcagacgacgaggacccggctctgctgctgcagacctgcctcacccaccccgtgcccaccctcctggcccagcccgaggccctgcgctccaagggcagcggccctcacgcgcatgcccagcgctgcttcctgtccagggccacggtggctggcaccggtgcgggctcaggccccagcagcagcagcaaatccaagctcaagcgcaaagaggccctgagcttctccaaagccaaagagctctcccggaggcagcggccgccctccgtggaaaaccggccaaagatctcagccttcctgcccgcccggcagctctggaagtggtcggggaatcccacacaggtaggtccagcgggaggcgggaggagctcctggttcccaaggaaaccggggcgggctcatgcgcccctgctgcccttccctctcctttttcatcttcctacttgatttcaagttaaaaaatgtggaaaactcaagggaagaacaaagacccatccatgaccca

SEQ ID NO: 395 - DNA, 926 bp, Homo sapiens
ctccaggctgggcgacagagcaagatcctgtctcaaaatttgaaacaacttcaaagaaggaaaggaagctatgcctgttgcatacaagcagaatcttcctcgcggctcggcctgctccagcctcccggaggatgagcctctgaccagggccggcccaggtgccacagtcacagccagtggcccgcagttctgaggcccctggcaccgctgaggcgtccctggagcttcctcaccagcctcacgcatcctgccggggtggcggggtcagggcccacctctgggcacgggaggcgtgggcctcacgggccacggatgggagcccagggcagggggctggagccctcgggggtcagagcccgtgcgccttccagggcggaggcggtcagccgatggaccaggcggtgccgcagcgacagagggggttgagatcggggctttcccctgcgaccagctcgcagcctgacgtcggcctcgctctggggaaaccaaggcacgggcacgcgcgggcgtggcgccaacgtcctcgctgccggccccgcctcgcccgggacagcgccccggccctgctccccgctctacggccccgcgccccggccatctgcggctcctcttgcttcgcgcccgcgccgcgctctgcccacgcccagccccgcagcttctctgtgacccccgcctgccgcaccccggctaggggcttattagcgccacggagctgagctggacgtgtccaggatctggggagggagcagcccagagagggcagggcctggccaggcctccccaggcagccctgcggtcctgcagccacgaggggaggggacgccacaaatccgggtgccgacccgggccaggagagtcgggaaatgggccttggctgggacccaggccgactctgatcccgcgggcctcgtagcccctccagccct

SEQ ID NO: 396 - DNA, 4391 bp, Homo sapiens
taggggacagagccctcccttagtgatgctgtgtcaccgaggatgtaagagtagaaacctttagctcacaacacctacccaggaaggaaggctggaacagcgcctccggacaccggaaccgctcattgccaatggtgatgacctggccatcgggcagctcgtagctcttctccagagaagaggaggatgcggcggtggccatctcctgctcgaagtccagggcgacgtagcacagcttctccttgatgtcgcgcacgatttcccgctcggccgtggtggtgaagctgtagcctcgctcagtgaggatcttcatgaggtagtcggtcaggtcccggccagccaggtccagacgcaggatggcgtgggggagggcgtagccctcgtagatgggcaccgtgtgggtgaccccgtctccagagtccatgacaatgccagtggtgcgcccagaggcgtagagggacagcacggcctggatggccacgtacatggccggggtgttgaaggtctcaaacataatctgagaagggacaaggggcggcttagtcagggacagagacccacggccaccccatgctcacacgccacaacatgctgcatgccagtgtgatgtgtggagaaaagaaaagaacgcaggcagaaaccaaatgagaaacctggaggcttcagggaggaaatgccgggagaggaacagagcctggaacagcgaaagaaacacttaaatgtcagaaatcaagccgggcagaaaatgactggggaaaggacgggaggagcacgggcgtcggccgagcctcacctgagtcatcttctctctgttggccttggggttcaggggggcctcggtcagcagcactgggtgctcctccggggccacgcgcagctcgttgtagaaggtgtggtgccagatcttctccatgtcgtcccagttggtgacgatgccatgctcaatggggtacttcagggtcaggatgccacgcttgctctgggcctcgtcgcccacgtaggagtccttctggcccatgcccaccatgacgccctgcaggggacgacccgtcagcctcgccggcgacaccgaacccaccccgcaacgcagaacccaggagccccgcggcgccatccactcacctggtgtctggggcgcccgacgatggaaggaaacacggctcggggagcgtcgtccccagcaaaaccagctttgcacatgccggagccattgtcaatgaccagcgcggcgatctcttcttccattgcgacctgcccggaaaaggatggactcaggcgggcgcgtctgtaacacggtcccctccccacagccacctaatgccctcccgcggggaagcctcggccctgccccaaccccagcggccgtggcctccaagatcgcaaccgcctggaaccgaaggccgggccttttacgtaacgtccacggctcggaagtctgcactgcggccgggccccgccctggacccccggcgcccccccagccccgtccgcctgacccggcccaccccgccttttgttcccggggaaggcgcgacgaggcctcggcagctggaagcggggccagccggggtcggggggcgcaggcctgcgacgtccagctcaggccccggggcggggcgctcaccggcagagaaacgcgacggcggagcggcggaagaacagagtgcgagagctggcagcggcgactgagaccgaccgcggcctcccccgccgttatttaagcggaagcggcgcgcccgcgcggcccggcggcgcgcgcggccccagaacatgtccatatatggcgatctttccgaaagccgggcacccattggcccgccgggcagggacacgtggggccccggcccgccccgcccccagcccgcaccgcctcccaacggccccgcccgcgccacccccgcgcgctcgcgaccctgcgcgggccggcgggcgggtctgagttggggcgccctccgagggtcgccgggagggccgaagggctgacgggcctggcccctccccgggactgccgcgccgtggggagggccctgctgcgccccgaaactgcctgacccggggcggggccgcgccggagctggggtgggtccccgagtccccggccacgctgcggggctttgctccctgggacgtcccttgcaatctttcccctcgggctccacgaggatctcgggcgccagtgccgggctgaggaaggggcgcggcgggggtccgcgacgctctgcgtccctgggcgcgggggagctctcaggagccctcacctcgccccggtcaccataggaaacagcccgcccgtccccaagcgcagccaccaccatgcaggtccctccacggggcgccctgccccggatctccgttcgtttgaagaccgaggcccctttctacccgacccgacacatccctgcccctcaaatcacaggcctgattgtccccaggcgcgcgctccgggttggggctaccccgcccccttatgtgctgagaaggtggtgtggagtgaggcctttgtttcattctggtcacctgtagacattatagggaaactgaggtagacaatgggagccctgacttttttgaaggtcggctccccagcttcgggcggtcggggggataccctcttctgtttacgatgctagttaagagcagcgatcagggctggcgtgggtttagccacaggcttgtccagggcgctcactcctcgccaggagcggcgccggtgctttggttacaccccgagaggcgcccggcactacggtttccttgtgcgctgctggaattttcagggttggagcgccggccgcgtaggggtggcagtgaggccgcgcagtgaggggacggactcgcccgaggtcgcagagcgttcagattgactcgacaggctggggggcggggccggggtccccacgcggagtccggggccctgcaacgttagctcgcgaccccgtgtttgggagtgacaacagcgagtcagggcaaggcggccggcccagctcacctgcgggctgcagtcagcccggacgccgagacccgtgcctaaggcccccgtgccccaggcctcccaggggtgctgagcgcccccaggcccagaatctccgggggcccgggcaaggctgtcaggtatgttccctcccgtggcgaagccggggctgggcctccaacagacccacccggactcgcgtccctgggcagaaccacacagcctgctcgacccccgggtgaggacttccacttggggggtcccaagatgcgacgtcacacaccgagggacacgaggcgggtggggaatagccacaagcccctccatcctgcgttcaccccacactacgttctgctccgggccggccggggaccctgcccctccgggtctctggtctcctccgaagcttctttgtctaggaagtgcggggaggtgccggccccgggcgcgcgtccccgtcccccacctttcttccccacgttgcggacccgaggccgccagggtcgtgggggcgggggcgaggctggcgcctcgagacccccgggttccccgccacccgcagggcctgagccgcggggaggggcggggaggggacgccgccgcgggccgggggtggagagcgacttcctcctccgcggccgcgtcacgcggcaggggtcaaggtgtggcccaggtacgcggcggggggcggtggcgcggccatatttggcgacttcggcgccgccggagccccgcccgcctccgccgggaagcccggcccggggcagcgccatccccgcccgccgccgggcctgagtcacggcgggcggggggcggaagtaacggacggggggcgcccgcccggcgctcctggccttatttagtcaaggaggccgccgattggcgcgcgggacagaggggcggcggccgggcagcctcggggcgtaggagtggcccggcgcctccgaggggtcggggcgggagccgggcgtggggggcctcggagcctggggagtcccgggaccgacggcggcggggatgcgggtctgcctgtccctgggatgcgggcgggcgggcagggcgaggcctggagcccgcggggcgcagggggctggaccgcgcgctgtgatttttctgtttagcagcggggcctcgcccaaggtcgcgcggggcctgtcctggatatgctctgggaagccagtggcgcagggcagccgccacccttctggaaccagtgccggagagggccgggcagctgggcctggggaggggaccgcatggatggagctgggggtagctgggcggcctccggagccgggagagtcggcagctgcacttccgccagaggtgggtgtgtccttcacatttcaggaagggagacttggggcctggagaagcgatgtgatttttcttttctagttcagtgctggttttgatggct

SEQ ID NO: 397 - DNA, 1514 bp, Homo sapiens
ggaggagggagcgcagaggaaggctaggccgattcgcagctgcctcctatctcaaaaacgtggaaggggctataactctagggctgggagggaggggagtcgttttcgctgactctccgcacggaccctctcctgcccgctagctggtcagctcctccagctctccccgcggcagcaggccggggagcagatgtcttgtccagtgtgcgcggggcaccgaggtagggaaagcctgtttaacccgtcccgcccctcctgctctggccagcagctcgcccaagcagctctgcagagcgactaggtcagggcctctccaggtgtctccactcgagggacggctggcgccacgcggaaaaggggaagcgggtcagggcgggacaagggcaagaggttactgctctgggcgctgtgtcctaagggctgcctcttgtctgcggagaatggagggtgccgggggtcaagcccggactctgtcagggtacccgagtcttagccacgccatccttccccagcgcgccgcgggggcgggcacagcagggcgggcgtggctgggagaccggctgtgcctcgccgccccgcccgtcccaggccgggaagctgggacttgagctggtctgcattcgggcaaggtgagccccgggatgtttctcaggcagggcccagggctgcacgccctaacttggtgcggggtcgcggcggtccagcccggctgcacgtggtcgcgcgctccttgcagctcccagcccgccggcccgcgctcctgcgccccctccccacgcagtcgacctctcctgaccgccaggtgcacgcagggcgcgggcctgggcctcagctccgcgcactccccccgggacgcggcaccggggcttcgggtgggcccaggacccgcactcagcacgcacttcccccgccgagcactgtctggcagcggggacgcacccccaggaccacgagctcggaggcctggggccgggagatggcgcccgtggagtgggtcccgcgagaaggccgggaatggcgggccgctgtcgttcccacctgggccgccaggtgggtgccgctatcgccgcgaggccgcacctgtcgcgctgccggagcccgcgcctgcccaggagcgccaggagggacccccgctcctccctccgccactgggcggggccggtcgctgagtcacggccgcacaccggacggtgacagaggcagggctgcctggggaggggccggtgacctcagtcagggctgacgcggaaaagtgggatggggtcacccgtggcagtggatgagggggtggcccgggcctccactcactgcccacgctagatgtccggggtccctcctgcgaccgaggacaggaggcacccacagaggaccccaacacgaggaccctggccctgggtgcggggcgtttttcccgcactctgctcccctagcagatggcccctgctaagcaccagctcccagaaaaggaaaggaaggggctgagaggctggatcgaggcaacccagcaggaaaag

SEQ ID NO: 398 - DNA, 1289 bp, Homo sapiens
gatcctcccgcctcggcctcccaagtggctgggaccacaggcgcgggccccctccagccaagcattgtttttctgacccgcaggatgggagcagggagagcgtggctggccgggggtccaccgggaggccctgcgaagggcaggccccggccaggcggggcgcggctccacgtctcagccggcgcttctgcgcacgggctcactactgggggcggccgggatcacggactcgctggcctcgtctccggagcggaggcgggcagcttgaaggagcgaccccgtgggccggcgcatccctccccaggccccgagacccggcgggagacggaccctccccatgcctcgccgcggaggcccgcgccgagccaggagccgcgcatccattggccgagagcgcggccgcccgggccaatcacagggcggccccggcgcccccgggcgcgccgtggggacagagtcaggcgcgtgcgcactcggccctccccggcggcctccaggcgggacgcggcgtcggcgcctgaagttggggctccgtcctgcctccctgcgggccgggaaaagggtctcgcacgtgcgccccccgcggtccgcgatccccagccgtcggcctagccccaggcgggtcgatcccgctgtccccagccccggaccagcctcctccagctgccgggtggagaggctggggggcttttcccgaggtcggcggcgaggctgagagccccggccccgccgcgcccgaggagacagcccttcgcgggctctaaaggccccggggcctccgggatgttccggggccgagttgtgcctgttgccgtttccggcgcccggggcctccgggatgttccggggccgagttgtgcctgttgccgtttccggcgccgctgccctcgggcaccgtcccctctggccctgccttggtctgggaggggtcgctaagggggcggagggcgctcctagggggccctgcggatctcctggcctcggcccccccgggtggccagagcggggctctgcacaaaggcctcaggtagccgcggcctcggttccgagaaagcccctcgcactgaccgggcctcagctgacccgggcagcctactgacccgaccctgacgcccgggccctccaggcctgtcagagcctccaggaaaggaaatgggctgcggggcgcagcgaggctggacagcgggcgctggcccaggactccccttgcccagaggagaaaatggaggctgagcctccggagagcccagctcgatgccaggagcactttgcagagggacacggatggg

SEQ ID NO: 399 - DNA, 1486 bp, Homo sapiens
ggtggaggcgacctgggctgcaggttgtggctggggtccttgctatgcttctggcagcaggaatcagagaagcagtaactggatccagctgagggcctggcggccaccgagggtacgggcggcgatcaggttgcggccgagccgtgagcgttccactggacggcttctacatccggaagggctgggccgggccgggccgggagcccacacggggttggaggaggggggcaggaacgagagaggccaaaaggtgcagggccgtgaagaggggcgctgggcccacgctatgaggaaaaggggatcaaccctgccctccagccccgtaggccctcaggccctcgggctctgggctctgtgcaatccgggagtggctgaaatccaagctgagggtaatgaaaggtgctgctcctaagccgcacgtctctgatccgccctccccgcccgccctctgctgcccgcgggccctctaagagctgcccaggctgctgccgcgggtcagaggcgggtcagagcaggcagggggttcgtgacgccggctgggtctgggggctgtgggccagccgagccgacccgggcttctgggggaccgcgggggccgtgagcactcagagggcgcatcccaggcccctccggggacccggccagcctgaagatgccgacgaacggcctgcaccaggtgctgaagatccagtttggcctcgtcaacgacactgaccgctacctgacagctgagagcttcggcttcaaggtcaatgcctcggcacccagcctcaagaggaagcagacctgggtgctggaacccgacccaggacaaggcacggctgtgctgctccgcagcagccacctgggccgctacctgtcggcagaagaggacgggcgcgtggcctgtgaggcagagcagccgggccgtgactgccgcttcctggtcctgccgcagccagatgggcgctgggtgctgcggtccgagccgcacggccgcttcttcggaggcaccgaggaccagctgtcctgcttcgccacagccgtttccccggccgagctgtggaccgtgcacctggccatccacccgcaggcccacctgctgagcgtgagccggcggcgctacgtgcacctgtgcccgcgggaggacgagatggccgcagacggagacaagccctggggcgtggacgccctcctcaccctcatcttccggagccgacggtactgcctcaagtcctgtgacagccgctacctgcgcagcgacggccgtctggtctgggagcctgagccccgtgcctgctacacgctggagttcaaggcgggcaagctggccttcaaggactgcgacggccactacctggcacccgtggggcccgcaggcaccctcaaggccggccgaaacacgcgacctggcaaggatgagctctttgatctggaggagagtcacccacaggtggtgctggtggctgccaaccaccgctacgtctctgtgcggcaaggtagggag

SEQ ID NO: 400 - DNA, 1405 bp, Homo sapiens; identical to SEQ ID NO: 394

SEQ ID NO: 401 - DNA, 491 bp, Homo sapiens
cccaggggccccaaggacagccggacaacagtgggcgggtccagagcacctgggacacccgagggccaggtgtctgcttcgtccaccctgggccttggtccgggcactccctgcggtccagtgctcccgggaaagcgcccgcttcctgccgtcttccctgtgggccccagagaaagccccggggtggagggcgtcccccagccgggcacggcgccgtggttgttgatagcacgaagctgacagtcttcagggctcggccgctgctttctggatgcataaagctcaccaccgcgatgaagagaatgttctcgaacagtgcatttccccaggggcccgtggtcaccctgtgctggatgaggctgctggcccgcacgcggcaggggtggttccgcagtgctgggagctgcctgccctcgggggtatctggcccagagtccctgcttctgggtggggctgtgcagagggtttgaggaagcttggagtcctaccag

SEQ ID NO: 402 - DNA, 1100 bp, Homo sapiens
agtgagcctagatggcgtcactgcactccagcctgggcgacagagcgagactccgtcaaaaaaaaaatgcggttttctaacgcgtttccctttcgataatgtcccttcgacaaatattgttttcccacaaccacccaagcaggccaagggacattcgatagcaatcgccctaaatctgttggcaggaaagaagcctcaacgcacaaaggcaagggtcgaaagcgcgggttcagcagaccgcccctacccagccaggacctggaagccgcgaggcgagcggggggtctcggaccatcacccccgcgtcccgtccacctttgagccccgcggggagcagagcagcggtcccaaggccccggggagccccccaccccaactgggcgccgctgggggcccgggaccggttcgctggggcacccactgggcgcgcactcaccgacggactgcagcgaaaggcgaagagcaggcgcgtcccgaagacgactccggctgccacgacccggtcaaagcgtggggcccgcgggcgcccgggctataaaccccgcctcgccgcccctccccaagcctccgcagcacgtgacccgcggcagcacgccctgtcccaagcccgagtgtcaccctcgggcacgtcccggcgccctcccccaacgccggccctcctagagtggtgggggcccggtcgggttgagagggtgtgctgcctgggcgaggggccccgccacggggagaggagcctgtcgggagccgggcgggggcccggcctggggaggctgggagttggaggtagccctgcctggagtcggggcgctgcctggagtcggggcgctgcctgtggtcggggtcctgcctgtggtcggggtcctgagaggcccggggcaaatggggatggcgcccaggccggctttggggttttgaaaaatgcaaacaaattcacaccaacttgttgaacggcgttagtcctcagaatagaactctaaaaatggaagcggtggctcacgcctgtaatcacaacactttgggatgctgaggcgagaggatctgtcgagcctaggagttcgagaccagcctggcgaagatttttattttattttaaaaaatag

SEQ ID NO: 403 - DNA, 1124 bp, Homo sapiens
aggtcgcagactcgcaccatcccacgaggagacgaggccgagcgagctctgctgctgccgccaacgccgccgccgacctccgccgccgcgggggggcccgccacagccgccaccgccggggggctcccttctccttctgcagccgtcgccgccgccaccggaaccgtcgccttctccatccccagggaaagagggagggcgcggacgggggaggggcgtggggctacgctctaccgcgacttcggccgcactggggccgacacagcaatcggtgccgcctccgccgccaccgcctcggtcacctctactgccgccgccgccgccgccgctccgccagctctcacctcgtctcgcgatactaagggcggggagcctgacgatgggagggagggagggagggagagggagggagcaagggaaggagggtgaggcgtaacagggagaccgggtgtcgggagaaccagaggagaggagactggaaagaagcggggaagtgtgcctggggcagcttccgaaggggaacggcggggacagatgaggggggcgcgattgcaaccagagcgcccgcgagctcccggatttcttctctttcctcgctatttcccgcacacacagccctgcagccctcccttcttccttcctggcttgtgattagaacgcagccgcaggcggaagtgcagtgacgccatcagccgtcgcctgagccgtggcgcgcgggtggccgaaaggcgaaagtgagatttcaagggccgggtctggggccccgcgggcgcgcagtgctgcggcgtgtggggagtacggggaggctggagaggagccggccccgggcagtgtgaggtgcggaagcgcggtccagacactttgctgacgggaggccggtccgccagatccgggaggtggggcctcgttctggactgctgtacgtagttttattcctggtcatttttttccaccgccaaatcgagagatagagctttggatcatgagagatggcgtcgtgttgggaaaagagccttctttacctgagtttaagctcggctcatagacatcctctacctgagtttacacgtgtgtaatatgaaggcagtcgattaggagatgtctaagatcattacttacacctc

SEQ ID NO: 404 - DNA, 915 bp, Homo sapiens
cctgagaaccaaagggccaacttctggccatgcataacgaagcagagcagtctcagggtagaaggcactggaaaagcgttgtaggacaatttttcccagtagaaacggtgggggtgggagggagacccgctcaccccttttgacccacccctgcagctcagccacgcctcctagggaggtgggcagccttgcaggagcccgcgcgaggctgcagtaccgccctccgccacatgagggcagcacggcctacccgtgcagcagcggcggcccccgcgcccctcttggccagagccaggcgcaggccggcttgcggtgccgtcgttcattggccgcggcgcgggcgctgccatgttggcggaagcggacccccctgtgccgtggaaactggcggtggccgcggccgccgagtcggtctgcgcagcctcctgcgttttctcgcttggatcttggcactgagaggcggtggccggcgggatggagaaaagtaggatgaacctgcccaaggggccggacacgctctgcttcgacaaggacgagttcatgaaggtgcgcgcggcgtccgctccccggagccgggccatgagggtgcctctgccctgtgccctctgtggcggtctcttcggtcggctgctcctgcctgcgcacgctcagtgtcaggtcctccgccgagacctcgctgcacttcgcaatgagtcttttggcgtcaggtttggcgggtgcagcctggagaaagcacttctgtaaaatcgtgcgtgttacatggaaaggcctgaaagttctgggcaagtggttttgagctgcatgtcaaagccactgttacccgttcctgttaccatgcagtgcagtcagattgccagtccccttgtcggaaagaagcttgtggaccggggccttggagctcattaagtgtgtgtt

SEQ ID NO: 405 - DNA, 1143 bp, Homo sapiens
tctcacccaccatcagaaactcaattttttcctaaggtgcatcctgcgttgttgttgttgttgttgttgttttaaactccctccaacagacacaactatacgcaaacgtacgctacaaacaattaaacggaaatacttagcaaccacccatccaggaaaaaactcacggtgctggggagcctcgtgggctccggggaggctgcctacgcagtgccttccgaaggtctgcgcgtccgtctgtccgtgtctgtcactcttgcaccttccgacttcctttccctccggctcccggcgggcggcaccctctagccggctctcaactttgaggagtttcaagcagccgcggcggcagcagcagccctggacgcagcagccaagctcttcgctggctcccgggggctctcccgggttcctctcattgcaggctgaaggtaccgcctcggcgctctggccgccgtgtgcccgcgcgcggggcgccccgctcccagagccggggccgcgggagggggcgcaggcagccgggcggcagcgggcggccccgcctctccgcactcgggaggctgcaggacccggggttcccgcgcgcccggggccgggagacgggctggggcgccggtcccacccctgcgcgtcctgcctcctgcgggcagcagctcggagcctgcgggagggagcaggctgggcgcgtggtggggggcgcgcgatgcggagggggcggcggcgcagccaatccgagaggcggccggcgccccctcataccgccccgcggcctcgccgcctcctcccgctttcctcctccagctcccacccggacctctagaacggccgcgcagagcggggagggggcaggggtccacaccagaggcccagactctggtccttgagtcaagatgcccaacccactcgcctaccaattacgtcccttcgtccagctccaagccgggcggtaatggggtccaggaaggctaaaggggacacgcccagccaccgagggaggggtctcacccttggagacccacttcgcggcggccgcacaggtcacgtctctctccccacccaggccaaggaatccccggacctcttagagctttgctttggggcaagggccaaggaggctttgcctgcgccaagtgcagttgactg

SEQ ID NO: 406 - DNA, 1763 bp, Homo sapiens
ccacacacacactatgctaggtcaagtccgaagagatctgaggccgggtttggggcgcacgtggatggggaagataaaacaaatgagctacagggaagatggttaggaggacagagagaagaacaacgacgacaaaaaaaaaatgcatgtcagataagaaatacgtcctaaagagacaagtttagatgaggaggtggggcggcgaacatgagtgcggaccgcacgacttagccctgagcgcccgattggcgcccggctcgccgagctcccagcgcgtctcgctccggctccccgcgcctccagggtacagaaaacagagtgttttgtagagctccagggtcctaaaggtggggactggggacccgcaaaagctggggtggagagcacagccctgaccatctgagcccccggagccagggcgtgagtgagcgcctccacacgccacccctgaagcccaccacgcacctttggcagagagggacccacctcgcttactgcgtctccatcggttgccttggcagtggctgtaatccatgcagttcagaatgatgagggcaaaggagaaaaggcgaaactgcatctgggcggtcgggcgggggagagacgcctctcaaagtctaggaactggagggttcgcccaaagagccggcgccggccgcgctgctggggaggactcagagggagactcgccactcacccccgggccgcaccggtcagttcagcgcgatcagcatctctccgccacgaacctgagagacaagaagcgaaaacagggtgtgtgggggatggagagagccccggggaggcagctgcgccttcgcacgtcccaatttaaagaagagagggaaggaggaagcgaacccactgcctagcacgagccgcggagcggcttaatccagagcgggcagctgcggacgagttggagaggaaggtgattataatcagtgaattagcaacctctgccgagccctaagctccgacccagcggcataatgcgttgtgccggcgctccggggaaaggaaagaactctaaaggcactgagagcgcagggctaaaagaacgctcttaggccatttggagtccacggcggtgtacccaagcgccgcagctgcggctgccttggtgtgggttgcctaccgtgcgcacgtctcgggcggcgcggcctccctaggcgcggtgctccatcccgggctcgggaagcgagccgcttggttagcacgtgggctggggaaaagtttgccgagaccggctgggagcgcctggcagctctggaattccagcggccgccgcggggttggcgacgccaggcaggagcgcggagcggcgggtgcagccactgggatgctccgtccccgcccgcgagcgcggtccccaggcaaggggtgcgcccgctcacactctctgcctccctaaccaattgtgtcgcttcctgcgctttctccacggtcacttcacagctaagatttctttctttccgagctgtagaaggcagaacgctctcgggaggacgaagtgatccgaagggatgtggcaagcgcactttccgatggagatgcagaccggctgaggcttgggatgcttctataagtaggtggtgctgtggtccaaggcatctaagtctaggctgcctcggagttcctgggtcctaaagccagaaacgtcccggtttccctgaggtcctaaagaacgttgacagccaacagcgcgcctaacttggagcgaatcctcttcgggctttccagagtgcgggggatagataaag

SEQ ID NO: 407 - DNA, 691 bp, Homo sapiens
cgggtgctagggcccgacagggggaccaccggcacaggtttcacctttcaggagggaggcaggctccggagaccccctcctcagcccctccatcccgcgccccccatcctgagcctgcaggggccgccagctggtccaatccccccactcgccctggaccctgtggctgccctccctctggcctaggcccaggctgccccggccaacctcgtgccgccggctccctcccgctccctctgcgcccgcagagcggccgcgcactcaccggccctggcgcccgccagcagcagcagcagcagcgcaggcagcgccagcagcgccagcagcgcgggcctcggcgggtccatcgccagctgcggtggggcggctcctgggctgcggcctggcctcggcctcgcggccctggctggctgggcgggctcctcagcggcagcaaccgagaagggcactcagccccgcaggtcccggtgggaatgcgcggccggcgcccgcaccccatttataggaagcccaggctgcaagagcgccaggattgcaaaaggtccaaagggcgcctcccgggcctgacctgtttgcttttctacactggcttctctttgagccttgaagagcctcggggagggggcccacctgggatgcagccgcagccaccaggggctgggtcccaggtgggttcccttccccaagcgtc

SEQ ID NO: 408 - DNA, 1279 bp, Homo sapiens
ggctcaagcgatcctcccgcctcggcctcccaaagtgctgggattagaggcatgagccaccttgcccggcctcctagctccttcttcgtctctgcctctgccctctgcatctgctctctgcatctgtctctgtctccttctctcggcctctgccccgttccttctctccctcttgggtctctctggctcatccccatctcgcccgccccatcccagcccttctccccgcctcccactgtgcgacaccctcccgccctctcggccgcagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactgaacgccgaagcctgcagccatgcgaccccacgccaccccgtgcctcctgcctccgcgcagcctgcagcgggagaccctgtccccgccccagccgtcctcctggggtggaccctagtttaataaagattcaccaagtttcacgcatctgctggcctccccctgtgatttcctctaagccccagcctcagtttctctttctgcccacatactggccacacaattctcagccccctcctctccatctgtgtctgtgtgtatctttctctctgcccttttttttttttttagacggag

SEQ ID NO: 409 - DNA, 429 bp, Homo sapiens
catccagaggaggtctgtgtggtgtggggcgggccaggagcgaagagaggccttcctccctttgtgctccccccgccccccggccctataaataggcccagcccaggctgtggctcagctctcagagggaattgagcacccggcagcggtctcaggccaagccccctgccagcatggccagcgagttcaagaagaagctcttctggagggcagtggtggccgagttcctggccacgaccctctttgtcttcatcagcatcggttctgccctgggcttcaaatacccggtggggaacaaccagacggcggtccaggacaacgtgaaggtgtcgctggccttcgggctgagcatcgccacgctggcgcagagtgtgggccacatcagcggcgcccacctcaacccggctgtcacactggggctgctgctca

SEQ ID NO: 410 - DNA, 700 bp, Homo sapiens
cgtttgccaagaggcacatgcggtgcccagagaaaccccagaaagttggacttacccctcctggcctgcctgatctttgggctcagcatgttcttgcagccgtccggccagccccgcagggccgttcatgctgtcatccacttgcttttgctcgtcctcgcgcctagtcgcccctcatgtcctgctcgttcggggccccgtggccgctggcgtcacccgtcaggctccctcggctacctctcctggctccggacggacggctcgccttggactacataaacctcgatgcattatttatccatcccagaattaattcccatccaagcggaccattaaagcctcagtaatcactgctgatcaatcactggaccagggcggggcagtccctccgcctggcctgggcgcacgcgtggctgtccaagctgagcgcgggggcggggcggggccgagaggggcggggccagccagagacgcggggcgcggtcctggcccgggagagggtactggggttccggccatcctttgctgaggctggaagaaatgcgcctttcagccgccgcctgggagccgcactccctgccaagcccgggtgggatagcgctttggtgagaaggtagccgagaccccccccccaccccagagcccaatccacggggacccgcttatgagctcactgttttacgtgttgaaagtcaggtgt

SEQ ID NO: 411 - DNA, 1064 bp, Homo sapiens
gtcacaacgcgaggaggtccagacttgaccccagacctccagactcgagctgggaagtggccgaggctcctggtgagccgcagggcagcctgcccagccccgggaggaccgcgtgggagggccgggagagcaggtggctgccccccacaagggggcgggctcagccggccagtcctgcccggaacccccggcaacgcgcatacgactacacctgctccggagcccgcggcggtacctgcagcggaggagctctgtcttccccttcatctcacgcgagcccggcgtcccgccgcgtgcgccccggcgcagcccgccagtccgcccggagcccgcccagtcgccgcgctgcacgcccggggtgaaccctctgccctcgctgggacagagggccccgcagccgtcatgctttccgccatctacacagtcctggcgggactgctgttcctgccgctcctggtgaacctctgctgcccatacttcttccaggacataggctacttcttgaaggtggccgccgtgggccggagggtgcgcagctacgggaagcggcggccggcgcgcaccatcctgcgggcgttcctggagaaagcgcgccagacgccacacaagccttttctgctcttccgcgacgagactctcacctacgcgcaggtggaccggcgcagcaatcaagtggcccgggcgctgcacgaccacctcggcctgcgccagggagactgcgtggcgctccttatgggtaacgagccggcctacgtgtggctgtggctggggctggtgaagctgggctgtgccatggcgtgcctcaattacaacatccgcgcgaagtccctgctgcactgcttccagtgctgcggggcgaaggtgctgctggtgtcgccaggtgagccccgaggatcgccctgccctggcaccagggcttctcggcgccttgactgacgagccacagcgtggcataaggggttcgcagagggaggttcagatcggaactgtaggtatagaaaaaggttggcagtgtttccttgaatcgcaaataatgatcttttaggaccacctgttgtggaagc

SEQ ID NO: 412 - DNA, 698 bp, Homo sapiens
ggggctacaggtgtgccccaccacacccggctcattttattaattcttggtagagaccaggtctctctctgttgcccaggccagtctcgaactcctggcctcaagcgatcctcccacctcggcctcccaaaggactgggattacaggtacgagtcactgcgcccaggcttttctcccttttgactcattttgaggcccccgtcgtagggtacacgccctgggaatcgggggtcttccagtatagggaaccaccgagatgcgggccctccgaagtcctagagagggcgcctcggccgccggcacccgccacacttccggcctttgtgggccgcagcgacggcggtctgcggctgtcggttctgtttgttgctgtcactgctgtttgttcttgccagcggctaggtgtgtagttgcttggggctcctagcacgaggcctctgtccccaggcacgcagcgccagtctccggctgttgcgccctgggcgccgcctctctccggcccccctttgtttcctgagccagctgggaagactaaaagggtgggtcctgggagcggcccatggttcgacccttcttttcaggcatccttggagcatgcaggctgcagatttcaggccgaccccgctagcaccaaagtccatgctgctgtccgtggcagctcagtttgaaaatggcctggtagaaagtccaggttgttg

SEQ ID NO: 413 - DNA, 864 bp, Homo sapiens
gaaggaaggaaggatggatggatggatgaatgaatgagcgggtgagcggatggatggatggatggatggatggatggatagatgggccagagactgggaacggtaatgccggccagacgggtccgagcgcctcgggagccccagccctcctccggctgcggggccgctgctcctgctcccgggccgtcacggagccctgagggagggggcgggcgagcgggggccttggggcggccagggcgctgggagggaggactgggcggtggggaccggaggggagggagggccttggaggcggagaggagggacgggctgggagagggcccggactaggggcggcgggcaccgcaggagctccgcgcggctgcagcgcgggcgggagcggggacgcgatgtcgccgccgccgcctccttgcgggccggggctgcgcctccggggctgagccgccgccagagccgacagccgagcagccgctgggcgctcccgcggcgcaggaggatgggctgcggcgggagccgggcggatgccatcgagccccgctactacgagagctggacccgggagacagaatccacctggctcacctacaccgactcggacgcgccgcccagcgccgccgccccggacagcggccccgaagcgggcggcctgcactcgggtaagtggccgggcccctgcagaccctcgcccgcccgctaccccgggcgccttggcccgggcactgagagaggagccggttccctgaggaattgatggcgcggcgggggtggctgggaggaagcagaggcaggacctgcccactgatcagtggacagatgcaagcccagggagtggggtcgaccagagccccttaccgaaggctg

SEQ ID NO: 414 - DNA, 2304 bp, Homo sapiens
ctggtctgtacgtgaaggctcaaaggactcttgacttgctgctggggtgagccctccaccccaccccaacccatcccacccccagaacatatggcctgacaaccaggagtgtttgggcatggctagcacgaggaggcacctggcctggcccaccaatctcctctccccagggatttcccagcgtcagtcctgcagccaccgctgatgctctggctgcggaaacccggggttgcccgcgggagaaggggtggcgggtcccaggcttcgggcagggggcgccagaggacgcccaggagcccggccggcagctggcgaggcgggcaggcaggcggcatctctcggcccgggcgcccctccccccggcgggcaacagacccgtcgcctgctctgcacgggccgcggcgcggaggacttcccccggctgtgccctcggaccaagctgactgcgggagtggccgccaacttcacagcctacaaccccctccggatcatgccggccctggggctgcgggagggtgcgtctggtgcccagctctatgaccaggggtgggaggatagggatgggggatggacagatagactccggccaaagatggacacgggagtcagggatgcgggtccagtcgggatctcagctggaggggagggggcgcggagcagacacgggggacacggccccaattccaagctgaggggaggggacttgcagcaagattggcgagggaaggcgggactccggtcagatagatatgggggagggggcgctaggggcagaaatagagaagaagcatggtcggggtacaggcgcgggggagggtagaaactatgggcagacatggatggggacaggaaagactccgggcagtgatgaagtggaggagaggagcagggtccggcattcagaccgtgacgggaaatagagggggagacacagttgagctgcaggttaatgggggggctctaataaaggtggggggcacactgcccggatccaggccgagatagggaaagactcgagacagcaagcacggtctgtgtcagaattctggagggggagactttagcatagatgggtaagagggtcggtccaggctgggggagggggggggccgagattagagattcaccagcgattaagggggcctcggccctaggtttgggagttccccagaagatgggggtgggcgaacgctgaacgaactcagttcgggctgggggagaattcagcagagacgggttaggagagcgccccgagtccgggccggtgcggaaactcgggaccgcgccggcagggctcggtgcgaggccaggtcggggtctggggccgagcactcacctgggcgccgtcagcagcaggagcgggggccggcgctggcgggggcggccacggggcgcaagtttgccatccctaagtcggggaccgggccgggcgcagggtaggtagctgcagccgcgcggagggccgaggctcccgctctcccgggcggcgggtgcagaaaaggcgccgcggagcagcgcggggcgggcgggcgggcggcgccgggccgggcgcgggctcctgccgccgccgccgccgccgcctccttgccgcgccgccccccgctcccccgctcccccgccccgagcaccgcccgcgccgcgcgccgcctcctatttgcgggcggcggccgcagccccgggctctgagagccggtgccgcatcctcttcggtctctgagcgccgccgccgctgccgccgaacccggttcctccggccgctccggccgctgcccgcagcgcgcgccgggccgagtgacggccgaggcgggacgcggcgcctgcgcgggccgggcccgggagggggcgcgcgccgggccgggcgcgaggagccgcgggagaaggggcgggggcgacgcgcgccgcctcctgttaaagggggaacagcgccagccgagccgatcgtgccccccacctccccgccttgggccccaggcgccggcgcgggagaagccctgggtaagcccccgcccagcccgctgcttcttggaccgcagaatgaggcgatccgcgacgcccctccagctatacccatgccggaggcttgtcgcgccgccagggtgtcccgggccctgcaggctgaagcatttgctcaccttcccatgtgcattgcatattgcatgtccctgtgcatgcaggtgccagtcatgtgcgctgaactttgcatatgcatacaggtgccctaagtgtgcactgccatgttgcctgggtaggtgcatgcaagtgccacacacatgcacggtgtattaacatgtgcccaagtcctga

SEQ ID NO: 415 - DNA, 1664 bp, Homo sapiens
aaggggaggagggaaggggaagctcacaccaatggacacacatcaggggctggacatgaaaaagagaccaggacaagccaatggccagtgcggggagggggaggtgcggggcggggggctccgcggacgccagacgcggcccccgggggaggggcgggccgaggggagggggcgctggggccgcgggctcaccagtggccgcagcgagcgccgcggcggtggcgtggccgggagagaagaaaggggtggcaggggtgggaggaaagggtgggggggagcaagacgtgcgccctgctcccccccacacacgcggactctaaaatgaaagattattcaaaaaagaaaaaaatagagcgagagtgcaccgggaggctgcagccccgggctggggaagcgcgggcggagggaagccaggtagagttgctcccggacaccctcctccgtacgcacacccacttcccctccccgccgccgccgcgctcgctccccgcgtgtggacgccaggggccgaagtaaaagccccgagcccgcggctgcgctcgggaaactttgcccgaggagaggacagcaaagaaaaatcacccgaagttgagagctgagcctccaagttacagctccgcagcgggcgaggggaggtgggagggagcgcacggcaacgcggaatccagcctaagtttggagggctgcgggtccggaagggcagcgcccaagtctccaggagcccgcgcggcctggaaagaggggaccggggagaggcaggcggcgcaggccggggcccgagggcgcccccaaggccgagccagggaccgggaatctgagcgccccgccaagcgactgggtctgaatgcaactgaaagcgccgagtccccggctcctaagggtcgcagaaagagagcccgaggaataaagaacgaggagccagagtctggcccccggaggggagcgccgggggcctgggcaagagaggcaccgaggcgtccacctctctgagaggccgaggacaaagcgcgggcagtcccgggagtcccagtccctgggaatgtgctcacggcgccgcgggaggtccccgagccgggtccctgggagggacacaccccctcccactccagagcaactgcaaaagcacagagggatgggaagaaaatgtttcttacgggcagagcacaactgttcctgtgctgttacccagggaaagagaaaatgggaatggagagagagcgacaggctcgcaggagcagcggggagaccacggtggtgagatgaccgcctcgggtcaccgccgcgctccccaggcgccgccggccaaggcgcacacccaggagccagcagccccctctcgcctttctgcagacgttccctggaaattagggaagacctgttggaaataaaaccctgtctctttaatgcacacacgcacacacacgcgcgtgctgccgctgtcaaaaagcgggcttcactctggcgtgggatactttcagttttacgtaaaatgtacagcaacagcaccccctgcgattggaaagtttattcagaaacgtgcctgtaaagtcccctggcgcctcccgcctcccagcctccctgcctgccagcaccactctgcagccgccgccgccgccgccggggagatcgcagtccgattccaaatctggaaggagttgggggga

SEQ ID NO: 416 - DNA, 433 bp, Homo sapiens
tctgccaggggcttttcttgtcttctccttggtcatcatcatcatcgtcttcctcttcctcgtgggcagatcttctctggtgggggctggctgctggctccgagggggcatccgcagtccgtctggtcgtctcctcctgcaggctgggcagctggccaccacttctccgactcgacccctccaacaagcatcgcagggcactgtcctcgggggtacagaccgtggtcccacattcgctaccactctgttccacgtcatccaggtacacgagctgcgtgtaggccgtgctgtctggggctcgaggctctttctgctggtgctcttggacgggcgggtagttctgctgcagagacaaagcatctccccttcccttccgggctgattttggttcattcatatctacgccagagtccaaactggcatcattacttcc

SEQ ID NO: 417 - DNA, 603 bp, Homo sapiens
taggactggtgggaaaataagagagcagacccccaacagaggttgcgagcagagccagggatcacagagttttatctgccctcggtacgctgatttccaaaacccagcctcatattctatactccaaagcgcactgccaggtgggccaactccagcccccacaatccgatgccaaggccacttcttgccacttcctgcccgcctccagcccgcccctaacaagctagcgcatgcgctgtaggactatttgcgcgccgttccccccggtgagcctgcacaccgtgcttccggcgcggagatgaacttccacatgccccggcgcttgttgatgacgtaacttccggttgctgtgctgagtcggaagtgggaacccttcggccgctgagattctgtcgtgtcgtcgctgctggcacttcaggctctggtaagacaggcaatagcttcaggggacggggtcgttgactttgcgttgtgatattttctgctggtccaatgccacttcaagcctgttcactcttatcttctccaaattccttctcctatgctttgctaaccagggaccttctccttcttgaacttcgaccctgtctgatttggaacccc

SEQ ID NO: 418 - DNA, 2106 bp, Homo sapiens
tccattctcccagggagaaaaagaaaacttccaaagccgaggccgcagtcactgggaaaacacagggaacctggtttgagttagcagcgagcacccctcccccagtcacacacacacacacacacacacacacacacacacacacacacacacacacgcacttcagtcctctcccaggaagctggctcagcctcagccccggctcccgcccagctctccagccctcccggccggggcactcaccggctgacgggatccccgcgggcaggatggctcctgggggacgcgccgggggcagggggcagttttgtaaccccggctgggggcgctccggagagggggcggggccagggacggccgatccgcgcgcacccgccccaagcgggcgattaaatcattaaccgaccgctcccgcctgcgatcccgctgagcgcgctgagctcggggcgcactagcccaggagcggccgtctccccggctccaagggggcgggtggctcgactgccctccccccgcaccccgccaggaggcgcctcggcctcccgggcttccgagggagcgggcggggcgggagcctggcgggggagggaagggttcgcggggcgggagtaggggaaaggaggaccatttgttccttcggccattcccttccagacagctccttccctctggcttcccggccgggtgacattgggtggcgtgtgctgcagtgtgtgtacgagagcgagatgtgtacacgcctctgcccgtacaggcggagaattcggttcttattcatggagaactccgggcgcacacacgcccccctcgcggagatcgcgggccgcgcgccgccgattgctgcgctcctgcccacaccgacacacaccccattcctgggaagggggaggccctttaaaaccggcccccaaccccctcagcggcacccttggccagggccgggagttccagcgtggagagaggaaatggagtcccggcaacgctgcgcccggctccctgccacctcctcctccacacatcccgctctgccaggacactaggtgcgcagcttagggtcccaggcaccgcggtgggcgcggtgaaaagcgcgagcgccgagaagtgggggctccgcgcccttctccccgcgccgcggcctcgcatccccagcttcgatcctctctgccccacagggggactcgagacccccaagcccacccagggcacccagcgcgaaggcggctaccacagacagaggcggttgcccgtcgcctacctcggtccccggagacccgagctctgggccggctgcgctgtccccttcgtgggctccgcgcggcggaggggctggcggcccctgtcagcgccggcgtttgtttccctctggctctcgggctttcagggctgggagcctccaggacactccccttccaggctccgcccgcgcccgccccgcgcaaccctccccgcgcacccctctgcgctgatgtcacggcgccgcagcagccaatggccgtcgggaacctctgcccgactgctcggcctcctcggttcaaactgaaagcctgagactttcccaactccgtatggggcagcctgggcccgcccggcttccgggagacactcccgccccagcctgcgctccggccgcgaacccccactggggcccccagggccaggggcgcgtctccctccgggggcctggcgcgaggctcttttgaggctcttccccattcgagccggggcaagaaaccaagaactccagcccgggtggcgttcctcgttcctgctccctcccggacctgctaaccacctggctgcagtactgtcgggagatggggcaggagggctggcgccaccagcttcgtgggtgccatcccagactctcgctgtaaacgagggagcggggaggagccggccagccccgaggccaggcttggactgggcgtcagaaatgtttctacagggtgacagcccttgagctccccgcatccccgtctgtgcctttgcctcctaagtatcagctgaggctaaatttgcaccaggtgaacgtcaaggaggatcaactcctgagtcccagagcctgaatccaagtcccaggccttggactctattctttttagagga

SEQ ID NO: 419 - DNA, 350 bp, Homo sapiens
ccctgaggcagagggtgaggagtaggtacaaggaagtgtagggaaatttatcttaaatgcgcttgtttactcatgttgtcctaaaaccgacctttcatccgagtgcatgactgctccctgaaagggggaacaataatgttaattacccgcagattgtgtttgccccaggcttttggcattatatctagctgctcttatgtgaaatacctgtgtctaagtactcctttcatcgtcactggccagagtctgaatgtgtctcctctttccccagatctgttcctcctgggaagatgcagaggctcatgatgctcctcgccacatcgggcgcctgcctgggcctgctggcagtg

SEQ ID NO: 420 - DNA, 927 bp, Homo sapiens
tcctcttccttggcttcccaagcccccagggcgtcgccaggaggaggtctgtgattacaaaccccttctgaaaactccccaggaagcctcccctttttccggagaatcgaagcgctacctgattccaattcccctgcaaacttcgtcctccagagtcgcccgccatcccctgctcccgctgcagaccctctacccacctggatcggcctccgaccgtaactattcggtgcgttgggcagcgcccccgcctccagcagcgcccgcacctcctctacccgaccccgggccgcggccgtggccagccagtcagccgaaggctccatgctgctccccgccgccggctccatgctgctccccgccgcccgctgcctgctctccccctctccgcagccgccgagcgcacgcggtccgccccaccctctggtgaccagccagcccctcctctttcttcctccggtgctggcggaagagccccctccgaccctgtccctcaaatcctctggagggaccgcggtatctttccaggcaaggggacgccgtgagcgagtgctcggaggaggtgctattaactccgagcacttagcgaatgtggcacccctgaagtcgccccaggttgggtctcccccgggggcaccagccggaagcagccctcgccagagccagcgttggcaaggaaggaggactgggctcctccccacctgccccccacaccgccctccggcctccctgctcccagccgcgctcccccgcctgccagcaaaggcgtgtttgagtgcgttcactctgttaaaaagaaatccgcccccgccccgtttccttcctccgcgatacaaccttcctaactgccaaattgaatcggggtgtttggtgtcatagggaaagtatggcttcttcttttaatcataagaaaaagcaaaactattcttt

SEQ ID NO: 421 - DNA, 2009 bp, Homo sapiens
cgaatggggaagcctccaccggcggttatctcctcctcctcctagcctgggctagagacgaattatctgtttacgaaatcacaccaaacaaaacaagtgccgaatgcgccccggacttttcgagggcctttcctacctggtcttctaggaagcggctgctgccctagacgctggctcctcagtagcatcagcacgagggccacagcggcgggcgcccctggcgctgcccactcccccgtgagccgcgggatgtgaaccacgaaaaccctcactcgcggcgggccgcacgcgcgccgaatccggagggtcaccaagaacctgcgcaccatgttctcgccgcctccagggccgagctcggcagccgctgcgccgccctttggcaccagaggtgagcagcgccactcctgcccccttaactgcagactgggacccacgcaccgccccctgcccatctccgccccgcaggcgcgcacccgccttccctgagcgcgcccgccccccaccttcacccccacccccaccccacccccactcccacccggacctccaagatctcggaacggctctgagccctgcgcacgcgggaagggctgccggaggcgcccgtagggaggcgcgcgcgcgggcggctcagggcccgcgttcctctccctcccgcctaccgccactttcccgccctgtgtgcgcccccacccccaccaccatcttcccaccctcagcgcgggcgccccgcggtgacggcccaggggccggacgcctggaacgcaactccaggcagctcgccccctagctacatccgtcacctgacacggccctaccaggaacagccgcgctcccgcggattctggtgctgctcgcgtccccgctcccctattccccttattttattcctggctcccctcgtcgaaagtcttccattcttcaaactagattatttaaaaatgaaaaaggaagaaaggaaagcgaggtcatctcattgctctatccgccaatcaggaggctgaatgtcagttttgaactaaaagccgctccgctcctcttctagatttggaaaacaagcgaaattaaactaaaccgctgcacgcctctgacgcgacatctggacacggcgcggcgctggcgctgccggagctgtcgacccggcctggcgccggactaggtaggtggagtcgcacccgggggtcccagctgggtccgggcgcccattcccctcccagctgcccgcgtcgccgagggcgcctggctgggacaagcaccgagtcctttgtgtctagcccatttttattttcggttttaaccttcacgacagccgcggagcatctgagcgcttcttcctctttcctcttcccccgcgctcccctcccctgctggccgctcccctcctgtcgccgcgtcctcgcgtagaatggttgtcttggcgaccgttggccgctgccgcctccacgctctgccccgcgcccagacaccccgactccccttgatcccgccgcctgactcctcggcgtacgttctctctccggtctccccctccatgtcccctcctccccttttcttccacatcaccgatcctttctggactctctcccttcctcctttccagctgggagacaggaaaagcggtcctgtttgggaacagtaaaagcagggcaaggaaaggaaggagcggcagaaaggaggggtgagtcgaggacacaggggcagccggagaatgcggaggagccgggtcctgagcgcggtctaagcgaggctcggctctcgtccaggaactcggacgcgggctcgccggctctccgcgcgcgggaagtcgagcccaggacgccgccttcaggccggcgcgctgacccggtgccccgacccggagcctgcggtctgcctggatccgtcctaaacctcgcgggctggacccgcggcctgagtgggtgggtgtgtgccagaggattcgggactaggcccagctccgggaacctggaaatgtggcccgcttctcagtggcttcctgttcatgcgct

SEQ ID NO: 422 - DNA, 3388 bp, Homo sapiens
cgcaaacaatgcaggacaaggcgatcttatcaaagaaaagccctttcaatgccttaataacaaccacattctgtttgaattaccactactgagcaaatggcggccccggctttcatccccctccccgccaggcgcattaacataaacacgaccctgcaggcgcatttgaatggggctgcggccccgaccccgcgcctgctatgaaagtaaagggaactaacgcgactttctccgcggtggacgctcggacacgcgtgaccgcctcaaacccactccaggctgccattggtactcgcccctttttacagatgaggaaatggagaatcagaccgggtcacgcagatagtatcaggcggggttggcaccggagcctggctggagccacagaggacacctccggagctgaggcgcagggcagttccctcagccgaacaccccgggagggagactcgcctggaagtgtctcggagcgacccttgctcgggtgctgccccttcagtgtcccggacgtgccgggcaacgcgatgaccagaccctgagcgctcttcgacccatgccccaagggccttggaggcgccccacaccccctgggaacctaggagcggggctatggaggggagggatgagagggacttgtcagaattcattattgactgcgaaagtcaccaccctctttccgggccctaaaagagacaatggcagggctgggaaggtgtatatcaaagcaagccaggccgcgtccccccctccacccccacaccttgacagatgacgctccgcagaggagcccggctccggcccgcgggctgcggccaccccacattaacatcacaagggcacccggcgccgactgcggccttgccacctcggcgcaggacttcacagcggcctctcatctgctgaccccgcggcctgggctctggcggcttccctcccgcccccttcccggccctcaccccgcggccgctctcccgggacacgcaaaggtcttccttagggcgtgcgccacccccgccaaccgcgccccagacaggggctgtagaaagtgggtctttgggagccaggatctgggagcccctgctaggagaaaggcgcctcctctaggagaagggaccaagccaaagctctgaggctcacccgaggctgatctgtggccttctccccggtcggcggcattcccaccaggactctccctagcatgtgcacagtgacgcagaaaccgatgcccagagacagcccccaaagggcgctttgatttatacagttgtaattggctataaacttctgcaaagtgcccataaccagcgtgcctgcccaattggggcatcaaggagcgaagcaagaaaagcggaagcgctgagggctggggaaagactttaaaaggggcactcaactaggactctccccaccgcacttccttgccacctctaggcacactcgaacaccagaaggggcagacccagagtcaatgagccccacagtccggcaaacctaggatctttttcagttgcacacacacactcacagacatccctgacacccttgaaaggaagctttgggagaaatgacccctacctggagcggcggcttaattcccaccagggtttcctccccgtctccctgctctctccgcagacgcctgcccctaggcttcttgcccctagccttccagcttttgcttgaaatgaccccttcacaggtcagaggttcagagacggggcgcgcccccgtcgccttagcagctggggctagcaccttcttcccagcgacagcagctctccctggtctgcctgctcgctgtaccttgaattggcccgagtggccaaacccggtcccacacacagcctagccatcctccgacccccccaggtcacttctcttcttagatggctcagatggggaaagagagagcggagataaatggcctagacctagcccaagaaggttcgagcctccctctctcctcggcggccaggagccacgcaaggaatgcatgccgcagcaagtgccggaggacgcgattctgccggcgtccagcagtgctcacgccgacattccccggagcttcccgaaggtgtcccggggttgcgaatgaggagctcttggaaagaggacgcctgaacacggtgttgagactcagaatattcactcccaggctcagtaggtcagagaagccccgacgggccagaggcccccgaatttgtctccgggccgtgcccctcctggcgccgagctcccagacagcccgggacgcccctgtgcgcactggacgcgcgacgcgcagcagccactggctcgacccgcgccttcccaagcaccctccgaaggggcgcagcctctgcttaccttggctgccgagggactgctgcgccggcttccgcatccactcgcacaggttccgccgctggccgccgggagacagctgctcggcggcagcggtggcggcgggcccaggagggccggggttgagcgtttgcagcagcccagaagcgcaggaaggcgcggcggccgggtggtgcgggtggtgatgcgggtggtggtgcggatggtagtctgcggggctgctgtagcccatggctgcggccggggagccaccgttgaggccgtgagccacggcgttggcggcggccgcggcgcctccgggcgcgtagccattccagtcctcccggagtggggcgccatacgctgccggccaggatggccccggggactgcgcgctgtccaagttcgctgccgctgcagctgcggccgccacgtggtaaccgccgtagtccgggtactgcggggggctgacgaagttctgcggcgccaggttgaggccgccagagtggcgcacggagctagggtacatgctcacgtccttgtccaggaggtagctcacgtacatggtggcgagggtccgggagcagacctcaccatgctgcctggggaccgacgctggaggctgccggggggcacgaagggaaaggggcgaggggactcgaggagcggcgggtggctgcgccccagcccgcggtgctccgctggctcctcgcggctcttctgcctccgaggcggtccctccctctggcctgcctcctccctccctccctttcttccttctttcctcccacctccttcccactaggctgcagaggcggggaagacccgccacaggctggcgtgcggagccccaggccggcggccttccgtgattaacgagtgtttacaagactctattagtaatgacacagacaccaatggttggagacgtcgaggcgcagcgcgcactctacgcacaacccctcgaaacataatttgcattttaaaagataaaggggagggaggctcgtgagagggcagcgacctgacacagctaaatattcaaacctttattgttaagagcttc

SEQ ID NO: 423 - DNA, 260 bp, Homo sapiens
gggactctctgtgtggtgctgacagacccaaggcccagacacagcagaggtccgtgctggggagggcgggtcgtcctgttatggaacaggggtccaaacaagcttgcttctcagagcatcttctggggaactgaatataaacagaaagggaagaggaggagggacaaaagagacagaaatgagaggggaggggatagaggattcctgaacagagaccgcacccatgacccacgtgaccctgggaaatgcttctatccctg

SEQ ID NO: 424 - DNA, 3083 bp, Homo sapiens
aggttatcctaaatactagagttgccgggctcccagctcagccccaagaattctcccctcctcgcagggagaagccaccgcctggccccctcatcttagacgcaccaagtccggcgcagaggaagggaggggacacgcggagcaggccaggctttcaggaggcaccggaatctcctagtcctggctcgcacggctcgggcaagcctcgagatccggcgaccccaaaccactccctgggtccccgccggaggctggcccagggcggtcccacagccgcgcgcctcacgcgcagttgcccatggccttgaccaaggagctctctggcagctggcggaagatgccccgcagcgtgtccagttcgcggctcagctgttccacccgcttgcgcaggcggtcattgtcactggtcagctccagcaccttctgctgcgtctccacgttgcgctgcttggccttgtcgcggctcttgcgcaccgcgatgttgttgcgctcgcgccgcacccggtactcgttgctgttcttgtccaccgacttcttggccttgcccgcgccgctgccgccactcgcgcggaggtcggggtgcgcggcgcccagccccttgagcgcgctgccagggcccggcaggccggcggcaccgagcgcgggcgcggggtgcgggctgggcacgggcgtgggcggcggcgtggggtgaccgggctgcaggtgcatggtggtctggccgcagtgcgcgatctggaactgcaggtgcggggcggccaggtgcgcgggcggcgggtgcgggtgcgggtgcgagggcggcggcggcggcggcggctggtaagggaagaggccggccagcgccagctgcttggcttcatcctcctcgcggggctcctgcttgatcaccagcggccgcagcgccggcgccccgacgcgctcgtacaggggctccagcctgccgtccaggtagccggcggccgcgcagccgtagccgggcgggggcccgtgcgctcccccgggcatgacggcgccgccggggcccgcgggcgcgcccgggtagtcaaagtcgccgccgccgccgccgcccgtggggcccacggccgccttggccttctcctgctgccggctgtgctggaacaggtcggccaggaactcgtcgttgaaggcggccgggtcgatgtaggcgctgatgtcgatggacgtctcgtgctcgcagatgccgcccagcggctccggggcggcaggtggggcgggaggctgcgcggggcccgcgccccggggaaagccgaaggcggcgctgctgggcgcgtgcggggggctctgcaggtggctgctcatcgggggccgcggctccgcctcgtagaagtcggccgactccatgggggagttagagttctcccggcatggcgagcctcggcggcctccagcctgcgcggggcgtcgccgccgcccacccggagaccctgctcgcccgcgcccgcgcacctccgggtcgcgaatggcccggcccgcgccggcccagcttttatacccggcaggccgcgtcgccccctagagtccgaggcggcctctgtccccgggctgcggcggcgcggcgcctgctgggtcctagcgcgcggccggcatggggcggcgaaccagcgcggcacagcgccgcgctccccaggcaggccgcggcgcaacgcccaccgcctccagcgcgcccagcagagccgcggcgctcgctccaagctccgcccccggcccggccgtcgcccccgcgcccacgtggtcggtagcgggggccccctcctcctgcctgccctaggcgcccgtatccagccacggccgggagcccaggagtatcccgaggctgcacggggtaggggtggggggcggagggcgagtcttggtcttgagctgctggggcgcggattctctttcaaagccagaaccaggcctgtcccggacccgcgtcccggggaggctgcagcgcagagcagcggggctggggccggtggggggccgtttgggacgcgcggagaggtcctgagcgcggtggctctgcgtctcctagctctgatctccaggctacccctgtgattccgcgcagaggtacctctcggaggacgccggggtcccatgggcggcgccgcgcagggcgctaggaccccgcggggagcggaggcggcctcggcccgggagcctggaggacctggccggtcgatccgcccgggctggaaaactttctttataattacttctccaggtcggagcgcgcggcttgctaggcgcgcggggccggcgctgttacccggcgtggagtcgccgattttttttcctgcgggaccgcggggccccccagactagcggagctggacgccggggcgagcacggggaggggcgcaccgagggaggagacaaacttaactctggggccgggattccgaggcgggggccgcagccctcgaggcccgaagccaccgcttcctcccccgcctccccattcaggtgggcgccaacggcgggagcgagggtgtccaggccgccgggctgccaggtccgagcacgcacagggagaactctgcccagtggttcgccgggcgctgtagtccccgggatcctagggaccgaggcggccaggccctggggccccttgagtgcggcagctaatgctctcaccgcggcgggggaaggagcttgccaccgagacccccagccacgtgcgtccctcgcattctttaccggggccggggtggcggctacggaccgtcagctgggcccagatggagtcttgggagccctcaagtgtctcctgtccttgcccgcgccgcccctcgccactggcgctgaggcctgacgccgcctgcgtcccggctagaggcgcgcttgcctacaggtgagggaagacccccttcaccgacagtggccttaggcctggcaaggcgccacgacccgcccaggagccccggagggggcacagctaaaaacaccgctggagagccccgagcttccacgacgatcgcagtaaagaagcagtttcatctgggcaacgcacactgcgctttaatcaagttcctattcaacatagtcccagtgattaatagcccaactgcttcgttttc

SEQ ID NO: 425 - DNA, 1011 bp, Homo sapiens
ttccccgcggctgcccccgggagaccgaagaggtggctggggcaggccgaaagcagaggaaaggttgggcagtgtcacccttgcacacccacagggtagacggatcgagtttcagcttaaaccgaggggtgtgggggaagggtgttgggttcagctagtccacgtgggtgctgtcctccacttggtgctgaaatctgggcggccacatccccggggcgggagggggctacatccccggctttagacgcgcgagtctcaggtcccgctaattacctggcgggtgctgcccacccctgccctcgcgcacctagcgcggtggcaggcgggaaggcggggcctgggggagccccacccctggagactgcggctggggcctccctctcctccgcccgcccgcctgccactagctcattgcgcctctcctgcagtctgattggcaccggctcccattccggctccagcctccaatccgacccccatttcggctgcagcctcggacctagctccggccctcggtctatccggttgcatcctccctccctgttccggatcttatcttgcgccagcgcctactccaggatcccgtagccagacctcaagccatggctggtcccttctcccgtctgctgtccgcccgcccgggactcaggctcctggctttggccggagcggggtctctagccgctgggtttctgctccgaccggaacctgtacgagctgccagtgaacgacggaggctgtatcccccgaggtaacagtgcctgaggcgcgggaggaggcgggggcaggaggtgatgggaacgaaggtgcgggtagaagtgagaatccgggcaacagagaagggctataatcacgaaggccctggagctggagggctgtgcagtctgcagacctcagtggggtgggggtgggggccaaaaccataaagcaagaacattcctggggacctgccaagaccagctctggccctacgagttctagctgcactggctgcccaaatccctaattgta

SEQ ID NO: 426 - DNA, 1678 bp, Homo sapiens
aaacaaaagcaacccacaaaaataatcctttatgtcgtgaaagtctaatgtacttcaaacttctcagtgtttcataaaaagtctcaagcctaagcagatatcactcgcacagtggcgcgctcaggctttgtctccatttcccgcaaaagcaacagcagaagcagcagcagcaatctgcctccaaatgcctcccaggcaccgagatcggagactgtgatgggtcccggaccggcttccgttcctcggggcccctggggtcggcgggacattaggcggtctctctccccgtggggaggcgcggagtccgccagcacccggggcccgggcagatggcgggggctgcggcagcggctcgccgggtccctggccgcgcagacgggctccgcctaagggcgagtggccacgcaggagcgcccccttcccggagggcgcgttctgcagtcaccaaacggcccccgagacccccgcagccggaaaggctggcgccgcgggactccaggcgcccgggaacccaccgccagccactagtcctgtcccacggcgcgggaacaaagccaggcgggtgcccgggagccggccccgcaagcccggggactggacgcgaccgggacaggcagagacgctcgcgccgcctcgacggcccctctccaggaaaacggtgagccacagcgcgaagagcaaacgaagcacggcccagcgccaggccagcccaagggtgccccgggggcccccgcgcccgcccgagccaggcgtaccttgttcttgacttcggccgagagcccataggaagggcccttgttgaagtgggtcatggtggttcgggcggcgggaagagacagcgctggggtccggggtctctcgcacttcgcttccccgctcctggccccgaggagtggccgccgcgggggatgctcgaactccctcctctgggaggcgcaggagacggccgcggggcgcgggcggtgcctgggcgactgggtccaactgggtgctacagagcctcgagctccgctgcgaagcacccggctgcctcgctcgccgcccgcacctcggttccccacaggccgcccccgcctccgcctgggccttcgaggattggccgccgggcccgaccaatggagggccgcctcctcccgcctctgcgccgaggggcggtgcggccaattgaagggccgcgggggccgctttccctcccgctccctccccgcgaggtccccggcccccgccccagccctcacgccaccggggccggcggctaggcggccccgcccccgggctaggcagggcgcctgggggcttcccagccacaggccattcacgtcaacctccaggcatcctccgcggcgggccggaccgggccggcggaggggagcggaagcccaccggggctggcccgccgctgacccgccgcctccgtctgcctcctcccgggctctgggcccagcgcgcccagatgccgggcaggacccctccgcccgctgctggaacaccaatagcagcgttggagcctctgggccggaggaactggggcctccaccgacacctgcttcttccctcacctcgcagccttccacacctgttgatctgacctctgccccgctgccgcccaggagtaacctaaggcttttggcctgattcgagctcactccgtctaaacctctaaatgcggtgcgaaaaa

SEQ ID NO: 427 - DNA, 281 bp, Homo sapiens
catgtagactctttgtggctggggagggggttagcgtccgctcatgcgtggcctcacactccgcgtgcctcctgctccgaccccgaggagaaactcccgtctgctccgacgactggcccgggccccttttatactgtcctgatggagagcagggaggaaccctgcccctcggagagggggagccggccgcccgtgccccagccaatcagagctgcctggcccggcccccaatttgggagttggaatggagagggggaggaggagggaagggagtccacccc

SEQ ID NO: 428 - DNA, 1292 bp, Homo sapiens
ctcagggcgtcggttagctgattggcaaatacagtaacgcacacatcccaaacgggacttggcaaaacggtgtgcatctggattaactgtcatacagtctccacgtcactcttccacgaggccggacagaagcgggcagaagtgagcagcagcaggttcaggggaaggggtagggtggccgggatgaatgggaggccagcgaactgacctacggcggggagggacaaaggatcagggagcgcacgctgggcggggaggccgggaggaataccccaggcggaggagccgggctcgggaccaggctgcgaactggtgacaaagtcggttatcgtggagggccctaagtaccagcgaggggccccgcgggggtcctctctgcctcgggagctgggctcaaccactcgccccgcggagggggcatcgcccggccgggggcacccgggagccgcagacccgaggcaggtactgggggcaaaggcccaggccggcacccccgccccgcagcctcccctgctgccgagtccactcaaccctgccgccggagtttggattatccagtctctcgccgcccgggccgaggcccgtcccggagccccacacaggcggtccgacaagaacgccgcagtgccggcgcggaaagtggggcagcggctgcctcccgtccggtcgtgcgagcggcggccgccatcgcgggccgcggcgggaaggagaagaggggccgcgccaggcgccactttacctgagaaaccagcgggagtagcgtctgctccaccgagcgagttttgatctccagtcccgagtcgagggcgaagcccgaagagccggagccgtagactgctccggcgccgccaacgccggcgggtccgggagaggcggccatggccctcggtctatcccgcagccgggactccgcgccgcggcgagcctgccgccagtcagcccacccgcccgaggcggcggcgacaggacactcgcgccaaccgagaccccgcccccaggggcccgcccccccagggctccgccgccaaccctgcgtccccgacgcccgtcaccgtatgtccccgcccacccccggcctctgccccgcccgcgcagtccagcctggtgtgggctccctgcaaccccaccccctaagccgggtcccgcccccacatggccccgcccttatctcttctcagaccccttccatgggttccaggcaccttctcgccagggtggcgaggagaaacaaaacacatctgtcacacagccacatggcctcctcttacttggacagacaagcgtccccagca

SEQ ID NO: 429 - DNA, 1805 bp, Homo sapiens
ttccagcaggaagcgcttgttgcatggcgcacaacaggtttcatgagctgggggtagatagccccatgtcagcggcagaggtgccaaggggtaccgggccctcacacatcctccaggggaattgagtgcgtcttgcttttctccacgtcccaggttaccactacaacgacctggtgtctccacagtacctgaacctcatcgcaaacctgtcgggctgtaccgcccaccggcgcgtgaacaactgctcggacatgtgcttccaccagaagtaccggacgcacgacggcacctgtaacaacctgcagcaccccatgtggggcgcctcgctgaccgccttcgagcgcctgctgaaatccgtgtacgagaatggcttcaacacccctcggggcatcaacccccaccgactgtacaacgggcacgcccttcccatgccgcgcctggtgtccaccaccctgatcgggacggagaccgtcacacccgacgagcagttcacccacatgctgatgcagtggggccagttcctggaccacgacctcgactccacggtggtggccctgagccaggcacgcttctccgacggacagcactgcagcaacgtgtgcagcaacgaccccccctgcttctctgtcatgatcccccccaatgactcccgggccaggagcggggcccgctgcatgttcttcgtgcgctccagccctgtgtgcggcagcggcatgacttcgctgctcatgaactccgtgtacccgcgggagcagatcaaccagctcacctcctacatagacgcatccaacgtgtacgggagcacggagcatgaggcccgcagcatccgcgacctggccagccaccgcggcctgctgcggcagggcatcgtgcagcggtccgggaagccgctgctccccttcgccaccgggccgcccacggagtgcatgcgggacgagaacgagagccccatcccctgcttcctggccggggaccaccgcgccaacgagcagctgggcctgaccagcatgcacacgctgtggttccgcgagcacaaccgcattgccacggagctgctcaagctgaacccgcactgggacggcgacaccatctactatgagaccaggaagatcgtgggtgcggagatccagcacatcacctaccagcactggctcccgaagatcctgggggaggtgggcatgaggacgctgggagagtaccacggctacgaccccggcatcaatgctggcatcttcaacgccttcgccaccgcggccttcaggtttggccacacgcttgtcaacccactgctttaccggctggacgagaacttccagcccattgcacaagatcacctcccccttcacaaagctttcttctctcccttccggattgtgaatgagggcggcatcgatccgcttctcagggggctgttcggggtggcggggaaaatgcgtgtgccctcgcagctgctgaacacggagctcacggagcggctgttctccatggcacacacggtggctctggacctggcggccatcaacatccagcggggccgggaccacgggatcccaccctaccacgactacagggtctactgcaatctatcggcggcacacacgttcgaggacctgaaaaatgagattaaaaaccctgagatccgggagaaactgaaaaggtgagctgaagaggctgtgtgggatggctcgtgtacctaggcacctggaacacctgaactgtggtgtgtgtgttatttgtctctgtgtgtgagttttgttcgtgtcgtgcagaggtgagatgaaggtcaagtccatgaaggtcaagat

SEQ ID NO: 430 - DNA, 1502 bp, Homo sapiens
cggcacaagctgggatcccagtacacatctcgggacggaagaaccgtgtttccctagaacccagtcagagggcagcttagcaatgtgtcacaggtggggcgcccgcgttccgggcggacgcactggctccccggccggcgtgggtgtggggcgagtgggtgtgtgcggggtgtgcgcggtagagcgcgccagcgagcccggagcgcggagctgggaggagcagcgagcgccgcgcagaacccgcagcgccggcctggcagggcagctcggaggtgggtgggccgcgccgccagcccgcttgcagggtccccattggccgcctgccggccgccctccgcccaaaaggcggcaaggagccgagaggctgcttcggagtgtgaggaggacagccggaccgagccaacgccggggactttgttccctccgcggaggggactcggcaactcgcagcggcagggtctggggccggcgcctgggagggatctgcgccccccactcactccctagctgtgttcccgccgccgccccggctagtctccggcgctggcgcctatggtcggcctccgacagcgctccggagggaccgggggagctcccaggcgcccgggtgagtagccaggcgcggctccccggtccccccgacccccggcgccagcttttgctttcccagccagggcgcggtggggtttgtccgggcagtgcctcgagcaactgggaaggccaaggcggagggaaacttggcttcggggagaagtgcgatcgcagccgggaggcttccccagccccgcgggccgggtgagaacaggtggcgccggcccgaccaggcgctttgtgtcggggcgcgaggatctggagcgaactgctgcgcctcggtgggccgctcccttccctcccttgctcccccgggcggccgcacgccgggtcggccgggtaacggagagggagtcgccaggaatgtggctctggggactgcctcgctcggggaaggggagagggtggccacggtgttaggagaggcgcgggagccgagaggtggcgcgggggtgccaccgttgccgcaggctggagagagattgctcccagtgaggcgcgtaccgtctgggcgagggcttcattcttccgcggcgtccctggaggtgggaaagctgggtgggcatgtgtgcagagaaaggggaggcggggaggccagtcacttccggagccggttctgatcccaacagaccgcccagcgtttggggacgccgacctcggggtgccgtggtgcccggccccacgcgcgcgcggggctgaggggtcgggggcgtccctggccgcccagctttaacaaagggtgctcctctccaccccgcgaggaggggcagctccggagacccggtcttcagcgagcggggtcttagcgccggggaggtctacttccttttggggttgccattttactattattattgccttttttttttcttcaaaaggactggagactgatgcatgagggggctacggag

SEQ ID NO: 431 - DNA, 848 bp, Homo sapiens
ggcttttcctgggggtttttctgtgtgcgtgtgtaattatgtgcttagttcacatcttcatagtgcgcctttgtgttttcctgggtatctaaccattgcacgtgtgcccgggacttcagcgataagtgtttcggtgttcctgcgtgcggatttgtgctctccggggaggtctgcggcccaggttcgattcctgcgacttgtcctaggcaggcctgtatgtgcgcggcggccgcgtgctgtacagtgtgagggaacgtgtaccaaacgctcgcgggatacctgtgcccgtctagccaagagtgcacccgtgtgcgcgagcgggcttctgggacgccgccgtggtcgggggcggccctgcgaggggagggggtcacagggactggccggcgccggccccgtgcgcacggaggcgggggcggggggcgggggccgcgaggggggaggcggtacgaaaagggcggcgcgcgcggcggcggcggcagctccccggcagcggcggtggagagcgcagcgcgcagcccggtgcagccctggctttcccctcgctgcgcgcccgcgccccctttcgcgtccgcaaccagaagcccagtgcggcgccaggagccggacccgcgcccgcaccgctcccgggaccgcgaccccggccgcccagagatgaccgcgaccgaagccctcctgcgcgtcctcttgctcctgctggctttcggccacagcacctatggtgagttccccggcggcccggctcgcgccccctctggggaagcctgcgactccccgccggccgcccggtgccccgcacgccccgtctcgtgagccccaactccgcccgtcccgcctagccctaagcc

SEQ ID NO: 432 - DNA, 4668 bp, Homo sapiens
agaagcgctggggagggaagaggcccttgctctcccccacctcggcctcgggtccccagaggctgagctcccaggttgccctaaaagttccttggttgggcggaaagcgggcgagtggcctggagagagggagcagcagccgccagcccagttcccggtgccctccccgcggggcgtcagagaagggagaggccggcgctcagggtgggaggagaagggtttgggttacagggaaaccggagctgggaaaggttcacgtttcacaacaaaggcagaagacggaccacgccgtccgggcccggagggagtgtgggggcgggtgggtaagagtaacggtcagtgaagaaagggggctgggaggcagcccctacgcggagtggagtggccacaggccctgtcctttttcctcagtccctctagtgccccccgcagtgtgattccccaacaccgatgggatttgggaaggagctcggaatggagccgctggaagaggagacgcgtgcggggagagggcccgggcgggtgcctgtcccgtccacacttagtccccgcgccccgcgggcgcctgagattgtgagctggtcccgggagatgtccgaggacctcggcgcgccggccccagcagtgggcacgggggaagagggctggtggacgggatgtccccgggagagctggacttgcgccgcccgaggcccctcacctcttgcagggcgcgccgcctccggccccggtccggtcgcgctgtcgccggttcttgaaccagttgctgacctgcgtgagcgacaggccggtgagtgtggccaggcggcgcttctcgtccggcgtggggtagcggttgccgcggtagcaggccttgagcgctgcgcgggagcgctccttgaagcagtagactgtctcctcgccgtcccagatggtcttgggcagcgggaacttcttgcgcagtcgatacttgtccactgcgccaagcgcgcggccgcgggcccgctcggcctcatggtagcgcgcgcgcaggtagaggtcctgcaggaaggcgtggtgggcggcggggaaggggcggctctcgagtagccggtagagctcggcgtactcgccccgctggaaggccaccagggcccgcgcgcgcaacaccgggtcgctgccacgtaggcgctcggccgggggcagtgcgcccaggaagcggctcaagcggccggcgtggcccgcctggagcagcgcctcgcagacgcacgccacctgctcgggcgagaagcggaggcccgtgggcggttcggaagcggcctcggggggcgacccggggacgcccggggatcccgggccctcagctcccgcagccgctgcgcccgccccggccccggccgccgccgccgcctcaccctcggccgcctgcaaagtctgcaagagctggcgcgcttcctcctcctcctcttcggtcgccgccgccgccgccaccgcctcccccccagccgccggccccgcgctcggctccgcaggcaaggtagccatgttttgcaactttgggaagttcctccctccctctcttcctccctcgggctttccccagcctcctcccccacctgtccccccttttcgcccccactccccgctcttctcgatcttctttctggccgaccctgcgccccacgccgggaaggcgagatccagctctccactcgggtctctgtccccttgtgtgtgtccgtccccctcccgtctgtctgtgattctccctttgttttccctccgcctctggccgcgctttctgcctccccccagcgtgtgcttctggctcagggcctcagtttccccatcgggacaacgcagaaggtaacgggccgtccaggaggactaagggcgcgaagcctccgccccgagactgagcttctgcacgcctccgtctccagggtcctctgcaggcccccacattccccatctcggcctgcgctccgcccctcggaattcccggctccgcagggggggcgggtctggccgggaggaggggcggggaacgggctagaaagtttgcagcaacttttctcgagcttgcgtcccaggagcggatgcgcgtggcgtgcgcaggcgcagtggaaggaggatggccgcgcgcgctgccagcccagccccctcttctcgacgctcggtggcacagctgggccacagctgggcgggggcggtgcctccgggtggcccgctcgccctcctattggccggacgccaaagccccgccccgtggcttttcctcccccaaccctgattcggccgcttcgcatcccgctagctcctcccagaccttcggccgcctccacacgcctccggattggcccgctgcggagcctccggcccacaacgcaaaccgcggacactgtggagtccagagctttgggcagatggagggccttttattcgcgagggtcgggggtgggggtcctaggtggggacagacaataaataccgaggaatgtcggggtctcagtgcatccaaaacgtggattggggttgttgggggtcctgtagcctgtcagcgagtcggaggacgaggtcaataaatatccaaaccgccgaagcgggcggagccggctggggctccgagagcagcgcaagtgaggaggggggcgcgggatccccgaaaaagcgggtttggcaaaagcaaatttcccgagtaagcaggcagagatcgcgccagacgctccccagagcagggcgtcatgcacaagaaagctttgcactttgcgaaccaacgataggtgggggtgcgtggaggatggaacacggacggcccggcttgctgccttcccaggcctgcagtttgcccatccacgtcagggcctcagcctggccgaaagaaagaaatggtctgtgatccccccagcagcagcagcagcagcagcagcagcagcagcagcagcagcagcagcagcagcagcagcattcccggctacaaggacccttcgagccccgttcgccggccgcggacccggcccctccctccccggccgctagggggcgggcccggatcacaggactggagctgggcggagacccacgctcggagcggttgtgaactggcaggcggtgggcgcggcttctgtgccgtgccccgggcactcagtcttccaacggggccccggagtcgaagacagttctagggttcagggagcgcgggcggctcctgggcggcgccagactgcggtgagttggccggcgtgggccaccaacccaatgcagcccagggcggcggcacgagacagaacaacggcgaacaggagcagggaaagcgcctccgataggccaggcctagggacctgcggggagagggcgaggtcaacacccggcatgggcctctgattggctcctgggactcgccccgcctacgcccataggtgggcccgcactcttccctgcgccccgcccccgccccaacagcctacagctgttgttagtccactcgcacgcctcgaatcccgtccgaactcgtcattggctgcttcctagcggcctgtgttgattggctgcccgaagatccgccctcctgccgtgggcccagccccgcaaatgcgcagctaagcgggtggcaaggggcgggtggagcgcggggcgcgacggcggaggggggcgtgggcagccggacgtaccctggcagggagcagcaggtggcggcggtgcatggggcctggccccaccagcgggcactggcccacagccacggccggggggccatctagctggagagagaagggacaggtgacccgatcggagcccagcccagccctcagcggtggggcgagagacagcgaggggaatcgaggttggggaggttatctagggagatcccggagggaatctggtgaggcctgaacggagggagatctggggctgaataaagggcttctgccctctaaagtcgcaaagacgtagggtgagccctatatctggacggggagaccaggagccagggaggggatctgcagaatgggcagcaggtctgaggcaggggaaagagaggggtcttacatgggaaggtggatccgtggcccggggactggggacccccgtgacagctggaaggagaagaaagaggcatagggcgcgtggaggggcgaaggagggcggtggcgcggcgtgccccagcgtgggtcccttccctcctccaggtgtctatacacgccccgcggagcagacggcccacctcctcccggtcctccggggaaggggacacatgagggactcacctgtggctccctctgcctgcagcaactccatccgctcctgcaactgccggacgtgtgcctctaggtcccggttccgagcctctgcctcgcgtagttgactgtggggaggtaaggacggtgagtccgtccgggccggacgagaggggatgccaagggttgccaccggcccgcatcccggccccggccccggccccgatcccgacctggcgaagttctggttgtccgtgcggatggcctccatctcccggctcaggctctgccgggtgagcacctcctcctccagggcttcctggagctcccgcagcgtcacctcggcctcagcctctgccgcagggacagccgctggaactgccacttcagcctgtgtatggggaccaggcttaaggctgcctgtggctcctggaagactcaggacttgggcactggttccaggctaggaatc

SEQ ID NO: 433 - DNA, 522 bp, Homo sapiens
gcccagcgtggtgagggctctgggggggcctggcgtggtgtaggcacaggttacaggctggagcagcagcagcaggagcagcagactcagcagaggccaccgaccaaacgtgccgggaccctcgaggccggagggctgcatgttgtgcagggccgggcaggcaggcaggggtggcagtcagagagcctgagaggggcgggcgaggggcagagcgcgacgatggagtcccgaccctcgattcaggccacctggcggcgtggcctcaagaggagggtaacgaagtcacagggccccgcgaaggatgtgcggagagcggcggcaccgcccagtacggccaccagggggcgcccagccctcccgccgtcatccccaagggcgctcccgccggctgggcggatccctgtcctgggcgcccactcgcggcctgtcttagggccctccaccctcccttttactcctcccactgtccccagggggccgacccaggtggctcctctgctgcccctaggctctctctctaag

SEQ ID NO: 434 - DNA, 4582 bp, Homo sapiens
aaataatccctctctccccgggagcggggcctaagtggctgcacccccggaagcccaagggccgaggtcctggagctgccgctctagggcggtcccctcgggatttcttccaggaaggaaagcaaaacacaacaaaacaaatccagactccacgtggggctggaacccagtcccagccctgctgtgttctagctggtcccgggtttggacgccagcgaaggaaggcgcgcccgcgctctccatcggggggactcgagggcagcgccgtgcggggaacatcccggccagccccggccattcgaggggacttgcgtcccagaggaattgccctcatctctcccaccagcgccgacaagctcaaacaatggcgcggacgcccaccctggtggcctaaccaggacctggccccgctcagccgccaggccttccgggacaagccccttcctggcactttctcttggagcgctttggggagaagggagggggactgggctcatttggaggagcaaaagacagccctcgcggaaaaagaaagaaaaatcagcaaaatcgccttagcagttcaaccaaaggtcaacagcagcgtttccccagcagctgacaggtcaaatgggcaggaaacaactgcggggaacaaagccccccgctgtccggcaggggcgaacccggctcctccgcactgaacagttttgttgtgctttttggaggggagaggtttccgccccctttccagctcccttcctcccgggtggacaggtcggaagaggagcggaaaggaaaggaagggagggaggcggcgaaggcgcctgcgggggggaggagcggctctttgatgggtgtgggggggtctgaactgcctaccgggctcggaagcgcagaccctacccgcgcggaagaatgcgcggaatgcggcggcgggaggcggaggcgcagcgcggcggggggcgcccggaccccacgcgccctgcccgctccctagcccgagggccgcggacccaccctgccctgcaggtgtggcgggcgccaggtccctccagggactcggacagcacgcgatttttccaggatttccgactaccaaatgtgacaagacagtctcgccgccgagagtttcgccagggctcgctaaaatccggcacgcgccgccatcagatagtcgaaaatccgggctgaacaaaaggtgatcattctccgctgcccggatttgcaggcgctagaggaccggttccgcccggcgtcccccagatacccagtccttcagcctcccgctcccagagcctggatctagcccgggacggggattggccaccttccaagctgagcttggagttgggcagcgggaacgcagatacagatcgctctacacagtgtgtgcaacccaatctccctttctgcatttgccagggtcttgttttcagtgaaactggcaaggccaattgtacctcggcgcccacccctcttcaccttttctccacctctgggattctcccggtccggctcagtgtagggccccagcacgccaaaccaacaggcctatcttgtccccacgcgccccgtggtacttggaaactgttcccgggactcaagatggaccgcgcagaggagtcgcttcatcctcgaggctaggaccgttaatgtcaataaacggggcctcgctaggacggtgcggaattgcaggtgaccggagaaacccgtccgcactttccctgaggacttgaacctcgcacttgcacagaaaccttgcaaacccctacacaccaacacacacaaaggggcgcgcggggatccccgcaaccacgaatcacgccggggactccttcccgtgccaataaggcgcaggccgccaagaccccctccatctccccgactccagctagcgcccggagccctcgtttacctttgagcaggcagatgtcggtgcgctcggcgttgcggcgcagcgcctgcaccaccagcgacacggtgctgtcctcgcggaggctctcggcgcgcgggctgcgctcgtcgtagacgatgaccgccgagtagaggccggagcgcaagcgggcgcgtacctcctcctcggcgggcaggatctgctccaggctcacggagcccttagcccgccgccgcacgatggtgttacagcgcacgttgaccgaacctaggatgtagcccgcgctgtgcgccaggaacggtctgcagtccagcagcaggcacttgccgccgctcggcagccccagggtgccgtggctgccgctgccgcccgcgccgccgccattctcgtcccggttcatcagccttttgagcacactgcagtccatctcccgcagctcctccatcgtcaccatggtcgccgggaaccgaggcggctgggcgcgcgaggaagagaagagaacccgggccgcctaggctgcagaaaggggcgggcgagagctaagaagggacgcctgcagaagtggccgcaaacttggtcctcaagggctcccgcgggagagcctcttcttccctgtccccttcctcccgcagcctcgcggtcacatagcagtcggagcggcctcgggcgcccagccgggcggcgcgcagagcggagggggaggcgccggtggaggagagtgtgtttacgagagaggaccgcggactcttttcggcgacggcctcccgtgtatttttgccggtcgcgcggctcctgtcgccactggcgccagcgctgccctgcctacgctcctccggcgctcagcgcactgccccagccagagttttctcctcggcttagaggtttattaatgctcctccgcgctcccgggggagggggcgctaggtactaccccgccaggccccgccccgttccattccgggccccgcgccgtcccccgccccccgcgctgtcccccgccccccgcgctgactccccgccccctccgggcccctcctcccaccccctccccggagcttgaatggagcgcggccgctggctcccggtcacgggaactcgacgtcacaaagagcggagtaaacagggcgccgggtgctcgccatcaggcccattcataaaaccgaggccgagaggaagagggaggcggcctccgacccgccgccagcggcgggcagcgcccggactttgtgaatggagcgcgcggcgcctttcgccgtctacccgcgggcccagcctgcgccgcgcctccagcccgacaccggcacctgtgccgccgctgcaccccgccgctaggcgggcggcgccaaaggaaatagccggctgaggaggccagggctcgggtttgagctttcatcccttggcaccttcgcgcttccccgcgtgacagccccgtaggtttttcccggttacccaagtggagaggggaccggagcgctgctgcccacccagcggggcctgacacctcctagccccagactccccgtgcgctttcggcccggccagtcctttctgaatatgcgattcctgagacgttagcgtatgcattagcgtgatcgattttcatctaaagccattaaatcagttttatagcttccccaagaacgtgggcaaccccctgcttggaagatgaaagagaaagtcctcccgcccgaagcccaggatcggagtgaggtgtggggtttgttgctttactacccaaagggaggcattaattgccggaatggtgttccgagggaggagagcaggctgcgctcctgtccacgcttcctaaaagatgtttatttaaccctggtcgggggaaaaacaaggttttcgggtttgggaaacgtcttcaccctatcttgcatagttgtgaattatcctttcaggaaatgacagggtccacatttgttgcggagctggaccctggaaaatgtacccgcaaagaatgaccttttgaaaggacttcagcagctctgggtccgattcatgtttctgggcatccctttttcctggtcacgtagcagccctccccatccccatttctcttttgcatttaatttcccagaggcggcttttggctgggggtttgtgcacgcacgcggtggggaccgtgcagaagccaaaaatcattcgcccgacatcttgccaagtcttgttagtgagaattaatcacagagcaagggagttacaggactcgctcgcagtttcggaggctgcagagtagcgtattccattccggggcgcgcgtggaagcccgacccaggcagcgagggcaccggtacccgccgggtctctcccgcccaagctcaccctctccccatgcctttgttccgggcggagcggctttcccgagctctcacctccggtcccgggggccatccctgcactccccgaccggagctcggcggagcctccacttccgagaccttcgactggcctcgtcccccactgggccccgaatgcctcgacccccgggccctactcggatgagctgtaggggcaccgcttgggcaaggtgatatttccttttgctccgggctccaaaatggcttacgatggcactgcacggatcctttctctttttaaaatttagtattttattcatcatggattttttgcatttttttttcttttaaagaggcattgcatcaagatatttcttttgattactgacttttcggcgcc

SEQ ID NO: 435 - DNA, 1510 bp, Homo sapiens
gtgagccaccggcggggctgggattcgaacccagtggaatcagaaccgtgcaggtcccataacccacctagaccctagcaactccaggctagagggtcaccgcgtctatgcgaggccgggtgggcgggccgtcagctccgccctggggaggggtccgcgctgctgattggctgtggccggcaggtgaaccctcagccaatcagcggtacggggggcggtgcctccggggctcacctggctgcagccacgcaccccctctcagtggcgtcggaactgcaaagcacctgtgagcttgcggaagtcagttcagactccagcccgctccagcccggcccgacccgaccgcacccggcgcctgccctcgctcggcgtccccggccagccatgggcccttggagccgcagcctctcggcgctgctgctgctgctgcaggtaccccggatcccctgacttgcgagggacgcattcgggccgcaagctccgcgccccagccctgcgccccttcctctcccgtcgtcaccgcttcccttcttccaagaaagttcgggtcctgaggagcggagcggcctggaagcctcgcgcgctccggaccccccagtgatgggagtggggggtgggtggtgaggggcgagcgcggctttcctgccccctccagcgcagaccgaggcgggggcgtctggccgcggagtccgcggggtgggctcgcgcgggcggtgggggcgtgaagcggggtgtagggggtggggtgtggagaaggggtgccctggtgcaagtcgagggggagccaggagtcgtggggacgatcttcgagggaaggagaggggcatccgtagaaataaaggcacctgccatgccaagaaaggtcgtaaataggagtgagggtcccggggataagaaagtgaggtcggaggaggtgggagcgcccctcgctctgaggagtggtgcattcccggtctaaggaaagtggggtactggagaataaagacatctccaataaaatgagaaaggagactgaaagggaacggtgggctaggtcttgagggggtgactcggcggccccctcccgggagttcctgggggctcggcggccgtaggtttcggggtgggggagggtgacgtcgctgcccgcccgtcccggggctgcgggctggggtcctcccccaatcccgacgccgggagcgagggaggggcggcgctgttggtttcggtgagcaggagggaaccctccgagtcacccggttccatctacctttcccccaccccaggtctcctcttggctctgccaggagccggagccctgccaccctggctttgacgccgagagctacacgttcacggtgccccggcgccacctggagagaggccgcgtcctgggcagaggtgagggcgcgctgccggtgtccctgggcggagtagggaggggttggaaaggggccgagaaattgcactcccacacccctgggttgcaatgggcaagctccctccttggctcaaacgacaccccttggaa

SEQ ID NO: 436 - DNA, 699 bp, Homo sapiens
ctttttttctttcatttgttgtttggggaggaggggttcttttttttttttttttttttttttttgcttctgccccagatctttcctggacagtgcgtctcagcagttcagatccgggggcccccagctgacagagggcgtggggggttaaggcattaacccctcccagcctcttcctgaagaaaccacccagccttggcgcggcgctgggtgacttcgcgtagcaggcagggaactggccgcggcgagcgggactggccattggagtgctccgctgcggagggaggggaccccgactcgagtaagtttgcgagagcactacgcagtcagtcgggggcagcagcaagatgcgaagcgagccgtacagatcccgggctctccgaacgcaacttcgccctgcttgagcgaggtagggggcggcgaatggcgggggtcgcgggcgcggggagcccggttgtggtagggcttaggtgtcagaaacgaatttttaagtcgaacgaggcaaacaaaaacttagaaaacatgcatctccgggcgctgaaaatttgcatctctgggctactaaggagcccatctcgatcagattgttcttgggcacttgtgatttatggagttcagaaaaaaggttaattcccaactgtggaacctacgggtgtaggggcagtgaaaattgcagttggtcggaggaataggagggaa

SEQ ID NO: 437 - DNA, 1031 bp, Homo sapiens
tctttagtgtccttgtaggacccacgtggtggtcatctcccgtcacgggggtccagtcttggctctgcgggcttcccaggggccacatagggagaggatccttcatgcatcagagtatcgcaggccagagtctatgccttatccccacctgtttggaaccagcttccaaaccgtggtccccagccttcagcaggcctaccgcgcaggcgcaaccaccagcctggcgcgaggcggacaaacgccgaggccgaggcaaggtgagagggcggggctggcaagaccccgcctccgtggcgtcacgtgggaggcggagtcggaagtgtgacggcgtgcgcggggcggggctactgctgcgcgcgggcgcgggggcggggcggggcgcggcgcggcgcggcgctcgcggctgctgcctgggagggaggccgggcaggcggctgagcggcgcggctctcaacgtgacggggaagtggttcgggcggccgcggcttactaccccagggcgaacggacggacgacggaggcgggagccggtagccgagccgggcgacctagagaacgagcgggtcaggctcagcgtcggccactctgtcggtccgctgaatgaagtgcccgcccctctaagcccggagcccggcgctttccccgcaagatggacggtttcgccggcagtctcggtgagtacggctggggagtcctgcgtcttctgcgaagggtaggaacttttttccttccgcacacagcccaggccctgccctcccgcctgcccctcggggtggcagcggctccactgcacatgttcccttcgaggctgccgcccctccgcggactccggtggactgagggcccctcccccatgtccaactctcctctgtgtcagaccctcctttcctcccggagcccctttcagtgcggggccctcgttcccccaaagtgggaaccccttccttctcaggccccccgatagctggcacacccctccccgtacactcacggctccctccacttaccccccttcagagttgggacatttc

SEQ ID NO: 438 - DNA, 736 bp, Homo sapiens
gggacatggccctgccctggagcttggagggacatggcctgcccttggagtctgaggtcccacccttggagtcttggggggacaaagccctgcgctggggtctgcggggaccctgctgtgatctgggagaggtccaagggatccctgacctcacaacccacagcgacacggtggaggaggatgaggacctgtatgactgcgtggagaatgaggaggcggaaggcgacgagatctatgaggacctcatgcgctcggagcccgtgtccatgccggtgcgtgacgtggagggtcgggcctggggagggcgtgggcggggggcagccccaggccccccaacaccggcctctcccctcgctctcagcccaagatgacagagtatgacaagcgctgctgctgcctgcgggagatccagcagacggaggagaagtacactgacacgctgggctccatccagcaggtgggcgcctcccacccagcgcctgccgggcgcatgcgcgggagctgggccggcaggtgcacgtccacctgtccggccgctctgggcagctgaggatttcctgctcccatcgcttttcctttctgtgactgtctccgtctctgagtttctctgacttacatatgtatatataaatatgagtctgacgtatatatatattaaaaaatatatataaaatataggacactgcctgcaggagcagtcaaaaaaccaaaaacaaacaaaataaatacaaaatca

SEQ ID NO: 439 - DNA, 1149 bp, Homo sapiens
gcaaggcaacagtccctggccgtcctccagcacctttgtaatgcatatgagctcgggagaccagtacttaaagttggaggcccgggagcccaggagctggcggagggcgttcgtcctgggactgcacttgctcccgtcgggtcgcccggcttcaccggacccgcaggctcccggggcagggccggggccagagctcgcgtgtcggcgggacatgcgctgcgtcgcctctaacctcgggctgtgctctttttccaggtggcccgccggtttctgagccttctgccctgcggggacacggtctgcaccctgcccgcggccacggaccatgaccatgaccctccacaccaaagcatctgggatggccctactgcatcagatccaagggaacgagctggagcccctgaaccgtccgcagctcaagatccccctggagcggcccctgggcgaggtgtacctggacagcagcaagcccgccgtgtacaactaccccgagggcgccgcctacgagttcaacgccgcggccgccgccaacgcgcaggtctacggtcagaccggcctcccctacggccccgggtctgaggctgcggcgttcggctccaacggcctggggggtttccccccactcaacagcgtgtctccgagcccgctgatgctactgcacccgccgccgcagctgtcgcctttcctgcagccccacggccagcaggtgccctactacctggagaacgagcccagcggctacacggtgcgcgaggccggcccgccggcattctacaggtacccgcgcccgcgccgcccgtcggggtggccgccgcgcccggcaggagggagggagggagggagggagaagggagagcctagggagctgcgggagccgcgggacgcgcgacccgagggtgcgcgcagggagcccggggcgcgcggcccagcccgggggttctgcgtgcagcccgcgctgcgttcagagtcaagttctctcgccgggcagctgaaaaaaacgtactctccacccacttaccgtccgtgcgagaggcagacccgaaagcccgggcttcctaacaaaacacacgttggaaaaccagacaaagcagcagttatttgtgggggaaaacacctccaggcaaataaacacggggcgctttgagtcac

SEQ ID NO: 440 - DNA, 1149 bp, Homo sapiens; identical to SEQ ID NO: 439

SEQ ID NO: 441 - DNA, 1099 bp, Homo sapiens
agggccaccaacagtcctcctcctcctcctcctcctccttctcctcctcgtcctccagatccagctgccaacagcatcccccgctcctgaagaaatgcaccgcccagaagggaacggcgaaagggggaagaagtccaggggacccccggcctctggccgagagcttgggtgggggcctcggccgtcgccactcacccggggaggggaaaagctccagatcgactttttccgtcttgatgatggtgagagtcggcttgagatcgacggccgccttcatggtgccaggagtgggggacgtacgggatggtagcaagtttgcagttactgttgtttttctttttaatgaggattagtaacagggggaaggggacgggggaaatccgactttcttcccaaaaatctcaaattcccgctgcctttctttcccccgcgcccggacggtgcgcgcccggcactccaggggaagttggcactttgcggcgaagtgagcgcgctcgggtcccagcctcgcccgcgccgcgcccgctcctcctgcccggccctcgcccgctccctcctctccgccggcggctgcctcgttcgctctcgatctcccggccgctctccctccctctcgccctcccgcgctctcccctcctctttagggcgtttctcgcggcgccgcgtctcggccgctgggtccgcgcgccctgggccgggcgatgtccgcttgggggagcgaggggcggggcgtcggggcagggcggggagccgggggcggggccgagcaccgcggccaatctccgcccgcggcccaaagcgaaaggaagggctggcgagcgcggggccgggccgcgaccgcggaggagcagtgcgtggagccccgggcctcggagcccaggcaggggcgggaagggccgggagcgggtgtgcaggctgggtgcacaggccgcctccaccgtttcgggaaagtccgtctgattctccacgcattcttgagggcttggtcacccgctctcgccccctcccagtagcagcatagttttccaagtgcgtgtacatcattcattcacaaagactgacacacaggacgtgaggcatgtggatgaaggcgagcgtg

SEQ ID NO: 442 - DNA, 619 bp, Homo sapiens
gcttaaaaaaaaaccgtttaggtagactttagagaaaggccctttcaaaaaaccaacagcaaaaggaaaaaaaaaaaaatccccacaatctagccaaagggtccgaatgtgactttctcgcgaagcagcacacgatgttggacagcaaagctgttacttggcaaggtggaactcggccgagagcggctccaaagtcgcgtggccagtgccaggaattcagattttggcaaccgcaccttgtgcgtccccgaaaccgacggacagagacacacggagggaggggaaggaggagaagagcgcaaggaaaagaggccgacaagccggcagagaaacccaccgaaggccggacgacccaccccggagacgtgtccagaccgcaccgtttcgcaggaggaacaaggaaatcgcccggcttagcaacgtagataagcagcagggctccgcgagactgcgggcgaggaggaaagaaggctgggggcggggggcgcccgtagaaacggtgccccggcaggaaactgtccgaagtgacaaactttcacatcgcccagactttttttccttttaaagtgaggagttctcttacctttagatttctatggcctcaactcagcaataatag

SEQ ID NO: 443 - DNA, 2242 bp, Homo sapiens
gtattacatctctgctcctccccaggagctgcccacaagcaccccctacttccaagacccctgttcttgtctcaacccgcccttctatcagggtccctacactccacttctgcccagctcctgtccagctgtgagttcacttgaggcctctcccgtgaaatctgccctctcggtctgaggtcagggaactcctgtaagacgcaaaaggcccacggttctgagccagtggcctccgaaggccggaatccgctagccccgggtctggcagaacgtcacttgctgcgccctaggcctcttcccgccacgcttagagagagggactggaggctcagaagggggaagagaaggtaacgcggcagctgctgggtggggacggtgactgtgcccctagatcgctgcctcctgggtcctgaatcttgctccccgctccgtagagttcccgaaaccccgaaggctctcaagcaccgcaagccactcctaacctcccccacgccattccccaagggcactgtcccccgaggctgggcgggcgggaactgtcatgggattcgctcgggcggagggcgctgggccaggacgtgggcgggcgcggggtggccgccgctcccccaagcttaagtcccgcgcagcccggggtggtgccggaagcccgtgcctgtggctccgggggaggggagggctcggtgggtccctgaagactgtcctccccgcgcgacgtcgcaggcgaagcagccccaggcaggacctcaaaaagcgacctgcagagtccgagcccctgaccgcccgcctgccgctcccggcttcgccgtgcacacgtctcccgctctccccggttcgtttttcgccacagctgctggattcgcgtctcgccagtggcgctccccgcgctgcattaggtagcgccgcgcggagcccggccctggccggaagcccaagagaggcgcagatccccggcggcgcgctgggctcgggaggcccctgggcgcacctgcccgggcggagcggagcggggcggggcgggcgcagggcgcgggcccggacgctggagggggctgtggctctcccgcgtccccgctgctcgggctgcgcggcgcccgcctccccccgccgaggggccggcccagacccggggaggggcggtggccactgcacttcccgctcgccggcctcagaggcggcgggtccggcgcgggcgcagcggtgcgggcgctcggctggggcgcggggcggggacgcggccgctgcccgctttgcgccgctcctccctgcgcgagtagcgctggccccggcgtcgaggcggccatggcgacccggagcccgctccccacccaccccgcctgctccgccctcccctccgccccgcgccacctttgatggctcggacctcagccggccaccgccagccctgctcgcgcgcccgcgccgccgccgcccgcgggtattaatagccggcgccgccgcgccctcggccgccgggggcttgggagccgccgatcccggagcccgagccgggagagggagccgccgcagccgccggcgctgtggaggtaggaggcgcgcggtgaacaatgaccgcggcgggagggcgggggccggcggggtccgggccgcgggcggcaacttgtgcgagtccaggctcccgcagcgcacggccgcggctgcgggcgaaggtgggcgcgtggtccccgaggtcctgccctgcgcagtcgggcggcgggtcgggcccgggcagccccggccaccatcgcaggagctcgggggcctcggggctccgggctgccccctgcgcccctctcccctcacctccgccgacgtcgggctgcggggctccgcgccggtccccgctcgcctcccccgaccccggcgcccttcccccgtttccttccgtcctacccgcccgctgacagcgctggacgccgcttccggacctcgggccgcaatctcggcccctgaggccggttgcgggccggggaggtggccgctggcgcggatgccgccgggtgcccgccgctcgcccacgcgcggcgcgagggttcccggcgggtgacaaagaggaacacaccctctgcccaagttagatttgtgtctctctttactgtctgcctttatgcagggcaaagttttttttattttttattttttaaagcaaaggacagattgctttgcggcgagtatttccaaacaacttctgattgtggttttacgattcaagttccggaaactttagctgtgtgatccggct

SEQ ID NO: 444 - DNA, 1682 bp, Homo sapiens
cacgtcctccgcagttcatctcccaccccatccgaccccaacccgcaccccaggctttcccgggggtggaaaaggacttaggaatgccccggatttttagcgttgagcccaaagctacctgctgaattgggggtgaatccccaaaaggggaccaaaaagacatggagtggaatgcaacacacacacataacacacacaacgcatcccacctcgccgcccactcccgccctcctccccagtccaggaaaccgcgggcctcgccgccttcgccccgggcctgtccctcccgggactcactgaggagccgccgccgcctcgccagctcccgagcgcgagttggaggaaaagttgggcggcgggaagagcggcgggacgagcgcagggagggggcgcaggagacgcggacggagggaagggaggggagacccaaggggcgcggatcgcccgggagggagccaggaggtgaaaggggcgggcggcgagcggagggaggcgccggccccggctgggctgcggcgggcaaagcgcgcagccgggaggctccggcgccgggggccgctcgggacgccaagcccgccccagatccgggggcgccgcggctcttacctcgctcggcgtggaggttccgcctcgggagagtccgccgtggcttgtgcgagcgggcgtgtgcccgcgtccccggctccggcccgccgcccccgggctctgttcgggtcctggcggggtcgcaagagctccgcggccggcgctcgactcccggcggcgctcggtgctcggcgcctccagcccgggcgggaacaatggagccggagctggtgccccggaggcggggcggggggagggctcccgtccgccacccggggtctccagaccttgtgcccccctcctccagaacgcagcggcggcgcctcactttccttgcagaccgggctccatcgccctgcggaggccccggcgccgcggcgtgcccgactgcagcacgggtaccgtgcgccctgcggggcgccccgagctcggaccggagaaaagtccttgggttccgcggcttttcaagcagcggcgcgctcgcggccggggaaggcgaggtcgccgcaatgcgctacgaggggtctcggtcccgtccggacgtggccgcccagctcccggcacacccgggttcctccgcgcgctgcggctgcaccggcgtcccggctcccgctagctgccgcccgccgccgaggacgccgcgcctgtggccgcaagagcgctccgagcgctatgcgccggcggggcgcgagggctcgctctctcactcccacccgcgctggtccctcctccgcctccgccccctgcagctgcctgctgggccttgtagttccccagccgcggccccaaaggacggggagcgggccgagctgtccggcgctctgcccttctcttcctacagcctggtctcctttggcgtttgcgcccctgcatctgagcacgtcccagagggcgccggaatgtcaggccctagacatagcagcggtttgggggctagggggcaggtgaagagacgccgcagccttgaggttgtacctggtagcgtgggctttggagtctgtgcgccttggggaaatgattttcgcgctctaaatctctgttttcccatctgtaaatgggggataatagtgcttttcacatagagttgaggcgtcaac

SEQ ID NO: 445 - DNA, 1405 bp, Homo sapiens
aacactcaatattttcttccctctatttctggtatgctaagatatcacacgagtgcgacttaagaaaaacttagaatcttttcatttaaaataagccatttttgttttaaccgaatggttcagttattaaatgtctacctgagtcaaggagcgaggtcacactgcaaactccacttatttctactaaagggaaatggcccggactcccccgcgcagaccaccgtgccaggacagcccgctcgggagtcgggcctggaagcaggcggacagcgtcacctccccgcagccgccggctgggacccgcggccagcctttacccaggctcgcccggtccctgcccgcatggcggtgccccacctggtgaggacgcgggctgcagtcctccctctggggatgagtcggtgggctcctctgagctccagcgcccccagggtctagacccctcccattgctccgctcccctctccccgtggtctccccacccccaacccccagggttcggctctcctctcccgggtctaccccacagctcaactcctttctcccggcttctccccgcacggttcgggtcccctctcccagtggtcttcccggacccacagctctgctctcctctcaccagtctccccaactccggctctgctcccctctccttgggtctcgcaccggattcggcccccacttccgagccctggtggctaagcccctcggcctccctccggggcgcagggggttggaggtactcacggcaccgcagcgggccggggactcccggacggggctggagggcgcgggcggctggtggctgcggctccgctgccggccgagtggagcgctgcgcagctcccgccctcgagaaaccccgccgtgtcatttgaccatataaggagatgcacgcctcggctcgctggcaccgggcgtggggcgcggggggcgggcgcccccaggcctcgcggttgccgtgcccccgccccgggctgcggcggtcccggcccgtaccctttgtttgccagggctcctttcttcgtgccctccgggtcttgggagcacagtagttatcgggagcgtcgcctccggcgtgggctctcgggcgcgagtttcggacgaggcctgggcgcggtggcaggggtctgcccacgccgggatctctgcctggtcccaggagcgggagactggagaagccccaggacgtgccgggggaggcggagggaggagggggtcacttctcaggaggcatgtgcctgggatatatttgcaaggtggacgtttttccttcttaccctgcagatgcttctttacgaaatgaacaccacagatgaccataaagaatccttcgttccactacggaggggactaaaggaaggtcctatccccacctctccctctcggggccagaagactagtt

SEQ ID NO: 446 - DNA, 2540 bp, Homo sapiens
ccttttgaaattttttcttccaattgacttggatctcctctggattttttttccctctttcccccgtcggactcttaaataaaagtgagcaagtccaaagcgtggtccggagagcgtggacaaggtaacctgttttggatgtccgggagaagggagtccggccgaaagggcccagcccctcgaggctgctccaactggggcgcgcggcggcccaggagggaggcggggaggtctccgggttgggccggggaacgccggcctccaggctgggtagggaggacggtccagagcgcgggcctccgggtctcaccccgggaaaagtctgggtctgctctgcgctgttgcgtgctgcgctcctgggacctgctgactgtcctagagggaacgtcccgcggtagtgtcagttccctggggaccaggaagggcgactcttgcctgggagtcagtgcttgggccgtcgggtccgggtaagcgctgggggtttggctcgagaagatgcggataggtggcctagggcgccgcggatctccgccctctgcgggcaccgggagcccagggtcgaggcgcgccgccccgaggggccccttctcctaacaatgcgcccggaatggagtccgcgccctgacagcgcgggtcagcccgaaaccgcccagaatccgcttcgatgagtgggtgagccgctctgggagtctgccgcgtctcggcagcggagggcctgagtggaaaaattcttattggatgcactttttaatggttggtttgcctcggttttcgtccgagtcttccccggcagtaggcgctgggttacccgcagccctagccaactctccctccataccccccctaccccacccccaaaaagtttgtctccggctggaaaaagcattaatgaacgtatctgtgggaagaaacggaaagtgaatgagtcaggagggccaccttcggccaaccgcctcaggaaaagcaaagaagccaaaggtctttggctttggattttgggggaaggtgatggggggaggttcagacttcgggaatcagggcgcggacgctgggcgtggaccccgtcattgttcccggctagctcttatctcccaggagcaagtatcctgtgtgcgcagcgcatgaatgtgtctgggcatctccgcgtatatttatatagtgtgtgatgcgaaaagcaggaccagcaggggaggaagagggggtgtggggggggagggaagacgagggagagcagaggggagagaagagagaggagagctcgaggcgagagagagagagagagagagagagagagagagagagagagagagagagagataggacttcctccccgattcgcaaagtgaagtcacttcccaaaattagctgaaaaaaaagtttcatccggttaactgtctctttcgctccgctacaacaacaaacgtgcacaggggagtgagggcagggcgctcgcagggggcacgcagggagggcccagggcgccagggaggccgcgccgggctaatccgaaggggctgcgaggtcaggctgtaaccgggtcaatgtgtggaatattggggggctcggctgcagacttggccaaatggacgggactattaaggtaagcggcggggcaacggacgcgggcggcggggaccggccggggaggcgaagcggcgggcgggtaggtgcggggcccgcgtcccggaagacgtggcctctctcccttccctccgcgccccggcttcgcgccggctcctccggcgctcgggtggcgagtcctactcgcgggccagccggccaggcgcccgcattgagggcgaccctcccccgaaatcccagcccccaaagtggagccccggcccccctcccacctcgctgcccgccggggctgcaggaggggacggcgggaaaccgggggaccccagggtacggaacggagtcaagggcgagagacctcggggtccaccactcccaccgacacccggggctcccgtggccccagcgctaggcactgggcttcctctccgcagaggcggaacacggcgggggcgggggctgcagtcgcccgggctcgggtccctgcctggtgggccctggggtgggtaagtggcctcgttgcacgagggtaggggtgcaagtgagtgtgtcgggggatctatctgagcgggctcctccagatggaagcccctgtttacatgtcagcgctttcccttcctctcggcactcagtcgcccggctctaggcgctggagagccgccggctccgcgggactcctgggccggcctcgccgcctctccggcggggaaccttccccagcccccgtccgcacagatccctagcgccccgagcccccgcccttcgcgcctaggcgtgcgccggctccaggaccagggctcctggagctgtcgcctccgaaagggtcctgcgtccttcggagccgcctccgttgcaggggccggctgtgagcccgcgccccgcgcccgcccggccattcccaccccagccgccgccgccaagcctgggtctaggttgcaaagtgcaacccaggcggaaaggtg

SEQ ID NO: 447 - DNA, 769 bp, Homo sapiens
gttaaggtcttcagcgtgaataaaaggcggcgtgagcactaaacgccgggggcaaagaacactcccggcccagagtttgacccctcctagcgagttcttcccggccagctgcccaactttttggcctctactcctccgcgggggcaccataagactccctacaggtgggctacacttaggggttcaaatgctgcattaccgcgctcgggtgagcgactgcccggcctgcaactcaagggactcccaggccaagtggctccgaactcgcgagctgtcggaacagtattatttttgctgctacgggccatctggcccagggcaacaggggctctgcgaggaaccatagagaagaaggggcgccagatcccgcgtgcctgcccacccgtccttcctttgggcagtcaccctgagctcggcaagctgtacctcacctgtccacctggaaggctgcccgctctccctgagggtcctgggaaggcggcacgagaccggaagtgcccccctcaggtgacgtcacagtaccgcgccggagctgatccgggaagccgatgtcagcctggcgcgcggcgcacagctggctgggagatttcttactggcttggagccggtggtccagatcagagagacctcttggagggtgtcggggttttcctttctttttcatttgaattttatccgccttttattttctgtcatccttgatgtgcttctggcttctggcttccctttccttctctctcatttgttccatgtctgcccctcttttctc

SEQ ID NO: 448 - DNA, 1036 bp, Homo sapiens
gccagcctgagacagaaattgcactcagcacaccctctgctcctctctgagaagaagccattttccagaccatatgacacgtccatctttcagggaaattagtatttctctgactccgacagcccccagagggtccccggcaggttcctcggcctctggacagacagggctgcctggacctggtccctggcaggttcctcggccccgtcttccggccacacagaagtccatccggtgacccttacagagcatgggtgggcgggtggtctttcctggctgggcaggggcgcactggccaaggccggccactgatgccccgcgcgccttgcccccagaatgcgctgtaccagtcctgccacgaggatgagaacgacgtgcagaccatctcccacaagtgccaggtcgtggcgcgcgagcagtatgagcagatggcccggagccgcaagtgccaggaccggcaggacctctactacctggcgggcacctacgaccccaccaccgggcgcctggtgacggctgatggcgtgcccatcctatgctgagccgcccaccgcagatgcctcccacgtgcgccagggaccctgtgtgcggagcctggcgtcggccaagccaccgggcaggaggcagccccggcctcccaagggcgcatctgagcaaatatgcaaaagcccacagggcaagacccaggctttcttacggttttccctggaaagagcgctccaggtgtcggaatccagtcccgtcccatcctctgcggagggtcgggctgtggccctcatgggtccccggcccgccccacccacagcgccctccgtttcccgcaccggcagttcacgggagatttgaatccaagccatattccctagtacctccgactgtctcccaccagggaaagcagaaatcaggtgtgttgtctatttatttctctatgtaaatattgtatttctgcggggaaattttatggtaaaaagtggaaaagggtttttccccatccgcgtgacaaggtgtgtgtgagcgtgtatgtgtgtgcgcgtgt

SEQ ID NO: 449 - DNA, 1154 bp, Homo sapiens
tcaaagtgatgtcattcatgactccgaaatggttaccatggcgactatcagacatattaagctgtcagggaaagagcctgtttaatccagtcttcaatcacgggtttcctcgggcgttagccactgcctgcggagtgtggactggcagaaaaacgtcaccttcgggccccccagcaccaacgcgccttaaaccaagccccacaaagaagtcacttcggcgatgtaaacgcgactttgcggcttcctgggctgctaaggtgaaggacgcccgccctccccagaacttgagagcttattggttggcgatgtcgtcactcagagtgacgtcaggggcgaggaaagaggcggaaccgtagagacttggcttcgggcccttctagcttgggggtcccgggaaggagctgggaggacctaggcggccgttccgcggagcccggccgaggaggtaggggtgggcaggcccagccctcaggccgcgacctggtgggccgcgctgcctttttttcctccggatcccgctcggtccgcatcctgctcctctcatcagagcatgatccaggcttctgacagcttccagttcccaccgcgctctcctccacccacgacctcatccccgccagttccccagcacagcgcgaactctgaagctcggcctctcctcccccttccccgcacagcgcgctccgtccttttttccgtcccaggccctgcttctttctcctcggcttcatttccaacaaccccgatcatccacgagggtcgtcgttcccgtgcctgtcccacctccgcaccagaaaaccaaagcatatcccccagaccccgccgcatgcaggccgcgcacacatcaggcgcgccgaagccgccgggcgaggaagccgagggcgcgttttgctcccgtgtggtgatgggcaaagccgagggagcccgggagcagcgggcgtggggcggcgccaatgcgagtgcgagtggtgtccgccgcccgggagcgcccgggcccgagcggattaaccgccgcctcaggtgtcgtctcctgcccgcgaaacaccacccaccgttagtgtgggtccacaggtttcgagcccctgttgaaaatgtcagctctgtggctaaggtttctattcaggagagctcctggaactcttttcccaaccaca

SEQ ID NO: 450 - DNA, 1342 bp, Homo sapiens
ctattcgttgatccaaccaaaattcggcggggctggggggtatcgttattgaggatgactcgctctaggggagaacgcggacgcattttcccatcagagttccctccataaatcaaaacctcaaaagaagagttaaagacgacacccaggtgtctacagtatccaagggccgggccaggagaggtccttggccacctggcgccgagttcgcggccgcagactcccacggacggcccagcacccgagcgcgcgggccgagctgccccagacccggcgcagcgcctcggcgaggccgtctgcgaagtccgggccgccactcgggaccgcggcccgagccagaggagagacttcggagaagagggaagaggacgcggggtgggaaacgccggggccgcaggcaagtagtgggcgagtccggagggcgcgaaagaggggaggggcgcgggaggcaatggaaggagcgagcgcggggaggagcgaggcggctgggccggaggaggcgcgcgcccgggtccacactgcggggtgggggctgagggaccgcgaggggctgcgagcgagcgagcggggccttaccgagcagcggcagctggccgccgtcgcgcgccaacgccggcatggcctccggagcccggggtccccaggccgcgccggcccagccctgcgatgccgcctggagcggcgcgcctcgcgctgcaggtggctctcttaaggatgcgcgtcaccgaccgcaaattccctcggactggtgcaaagtggaaggggggaggaaccctctccccagaggcagggaccgaggcgggagcggagaggttgggcagagccgagcagccggtgcctggcgcagcgctgcagccgagcccacgtgcccgccgcggtccgcagcctgtgctcggcggcccccggggctccccgactgcggcggggaagattgggcgcggtcccgccggagaagttgcactggctccgcggctctgccgccttgctgcccctgtgagtccccgaactctgtcgtttggatccggggcgtggggactgcagggagccacaaaccaccctcctagtgcgctgggatctttgaggccctgagaaaggcgctgaatccgctcggcacgctggcaactcccgccagcgcacaggcgagcattccattaagaaccattccattcttaattgtggactgatttcctttgtctctgcctccatctcctaggatgctcaggacccagtcagcggcgggatcaagaaaaacaggcagggtttcaatgtcaaccaaggcaattagggaggttaacaaacagccttcaaaacaaaacacacagaaaatacatcccagaggatg

SEQ ID NO: 451 - DNA, 1609 bp, Homo sapiens
gtagctcggattgcaggcgcgcgccaccacgcccggctaatttttgtatttcgagtagagacggcgtttcaccacgttggtcaggctgctctcgaactcctgacctcgtaatccgcctgcctcagactcccaaactgatgggattaaaggcgtgagccaccgcgtccggccgttcgtgtctgatttctatatgtgctgccgaagcgagcactcgtgtctaatttttgatttccccacaccacgcacgctggactagatcaccggctgggcaaggaagggggtctgcgtccctgcggggtcctggcagctcccgcgccaggactttgttgaatgaatgactgaactgagcgggcgccgcggggcggggcgtcccgaagcggacctcaaggcgggcggaggcgagagctccgccccggaaggcgggggcgggggcggggcggcggccgtgggtccctgccggccggcggcgggcgcagacagcggcgggcgcaggacgtgcactatggctcggggctcgctgcgccggttgctgcggctcctcgtgctggggctctggctggcgttgctgcgctccgtggccggggagcaagcgccaggtacgcgggactccggggtcggggaaccccgggccggggctcgggagccggactgggtgtctggagaacagcgccgaactgggggaacaggcggatgtggggatctggggtcaggtggccttcaaacagctcgctcccggctgtgggaagttccatcactctccaagcttcattcacaatccgggcctcagtttctcccaccgtccaattggggttggggggggtcttcagttccacaagtccgaagtacttcgaatggagagagaaagtgagtgagggcgggatgtttagtctaggaaggggcacgcccccagcctggtccgtacagggtctaactagccccagtgatcgacgagttggggaggccgtcgagaggcactagggtctgtgccctctataccatgtggggtgcccccttccatccggtgacttcagtgcccagctatgcggggggagggactggggtcgggcccggtgcccaggagtgagtcacccgccggcggcaggggggttgtggggacctgaccccccccacccccagcgtcccgcgctacgccctccctcccggagctgccgtgtcccggtcgggccgtgcggtcacctgtgaggaatgcgcggggagggcgccggtgactcacgttattcgagccggccgaccctgacctcagaccccagaatagccgagtcccgcccccagcctctgacccgaggccccctccccaggcaccgccccctgctcccgcggcagctcctggagcgcggacctggacaagtgcatggactgcgcgtcttgcagggcgcgaccgcacagcgacttctgcctgggctgtgagtggggggcagggccagtggccagcggaccccagactgggagggagggtggctggtgcgcagagcggggccgagagctttgcatctgggaaatcattcgggaggaggtgggagctgggagggggctccggtcagggaggaggccacgtttgggagaaggcagaaggctcagtcttaggggg

SEQ ID NO: 452 - DNA, 3649 bp, Homo sapiens
cgggaggctgagacaggagaatcgcttgaaccagagagtcggaggttgcagtgagccgagatcgcgccacggtactccagcctggcgacagagcgagactccgtctcggaggaggggagggaggtgtttagctcaactagacagcctttacagcaattccgccaccggtgacagtgagaaactgtcagcttggggaggccgcgccacgactggaccccgcccccgggaagcctccccggagccgagggcccccgggccttctcctcggagtcgggaagtgcctgcccccacgctaagtctctcctggccacgctctgaaaggcaaaaatggcaaaaatcctccttcaaggcaccgctccctcgacgcgcaccgccactccgactccctcaaaacattggtcgcaaagcccccccgcctggtcctccatgctcaagaatagggtggcaaggcaggaaaagactcacacatcccagcccccggcccctcagggcagcctctgtgtttctaactaaaagcctcctaccaggggcaagaccaagttcgggcgcttccccgcatggagaaacgctggccccacggcgcccggccccgacgcgctggccctttaaaggcgcgggcttcctgcgcccggggcccctcccacgccaccgccaccgccaccgccaccgcccgcgcccgcccccgccgagggtcacacttgaactttagctcgccccgcgcggccaggcgccggcgctccgcaggcggtggccgccccttcgcacgtgtcggaggcaccacctcgggctcgggacgccgggcccccggcactcgaggccactccggagaaaaccaaaacaaagcaaaatgcgcaccgcctccagaaactgcccggcaggccctgcaccgtccccggccggtcgcgccctgcctgtcccctccacctgcagcccggtcccggggcagcgaggctccggggcccctcgccgcgccctcccccgccgcgcccggcggaaacagctccgcggcgcggccggagagaacccgccctccccccgcggaggtccgggagggaaggggcagccgaagcagtcggcgcgggccgggggttgccgctcccagcgaacccctttctcctttcactggcaaacttttcggcctcgctctgacgtccacttcttggcgcactttctttacttagttccccaacgagccccttaccgcgtcccacgcgaactcctgactggcgcgcacgcacacctactgccgtccccgaccggacccgggcgaggccaccgcgaccaccgcttctcgcccgccctcctgggaacgcgctgccctcctgctccgcaccttcaggccgagcaaacctgcacagctgcgccctcgcctgacccaccgcgcccccaaggtccggccgcgcgccgagtccactcaccttccagcccgccgagctgttgctgtcacccttatccttgaagtagggcacgctcttgaccatccactcgtagatctgcgacagcgtgagccgcttctccgccgagctctcgatggccttggtgatgaggtcggcgtaggacaggttgccccacgcgttgcggcgggacgagctgctcttgcgcggctgccccgcgagcggcccagcggcggcggggggcaccggcgggtgctgcgacagcggcccgggcggcgggggctgcggtggcgctgggtgcaggcagcccgcctccgggccctggaagtccccgcacagccccccggtggcggccgcggcggccgccgccgccaccgccgccgccacggagccgggcgcctgcgggaagtcctcgctctcctccagcaagctcaggttgctcatgaagtcggcgctgacagcggcagccgaggccgagggcaggcccgccgcggcgtcggggttggcagccgcgctgcccgacggcgccgggctggaggtggccgagttggactggctaaactccggcctgggcagcggccaggtgcacgagcgcggccggggcagcggctcgaagtccgggtcgatctccaccacctgaggcgcctcggccatggtgacccccgcccctcccccagccgcaggagagccaagagggggagaacgcagcactgggggcggacggggagggggcgcgaagggacggtccgagatttgggggaacgaagccggtgcggcgagcggacggaaactgggaggaaggcgcggcggagtggaagcgcgagcccagaacttaacttcgcggggccatccacatcgaggctcctcggggtccgccgcacggactggacggccggccagagccgccgggccggggcagagcctgcgccgcgctccagctgacagggccgcggacggaaggacggacggacgccgcgggccgcttgctctccccagcggcgcgcccgctgcgctgctgcctgttgaatgtggcggctgcggcagcggctgctgcgactaccaggccgcccgacttacgggatctgccgccgccccccgcccgcggcggcgcgcgcgccggcccgcccctgaccgacagcccgcgcggccaatgggcatgcggcaccgccgcccgggcagccagtgggcgccgggctgggtggggcccggttttccacggggaggcggcggtgggctggtggggggtagtggggtgtttttctctttcacacactcacctcctttttttttttttggatctctattattttctggtaattctcgagtgtttctgtgattctctcgccttctcagtgttttgattgctaggaagcaaaccagcgtggaggcgccggcgacactttgtttactacggagcagcagagccgagtactcgggaagcccgggtgggaggaggcgctcgctgctccctgacctccgctgcgggccgagcccggcgggctggcagggcagggggccgagggccgggggcgcggggtgggcgggcggaggcggccgcgaggaattctactcaatcgctccctcctggctccacccacgatgtctttgctgaacgacgtggggaatcggtgggttttgttttggtttaatgttttctttcgctgcgatctgtcaagtcctccggccccctcgcgagcggcacacgccccccacccccggcgccgcggctcctgcagtcgagtccgcgccgagggacccttctccgtgcccaccggtccgcacccccgggctcagcctgtgccattcggtctagccagaggcgcgtcacccagccggcagcccccagccggattcactgtattcttgaccttttttaaaatgctagaaatgaggaacaggggctccggcgcggggaggagattatctggcctccctgaacacacgaatccaaaggcacccgcgaccgcatgccctccgtgggaacctcttcccctgggcgggaatctcccgctccgtccactaagtccagggccggcagcgaagggctcaggagcgcagttagagagaaccaggtcattttattacttatccctaattaaatttagctgtttgttacttcttttttatttgcaacggaagtgatcgggtgttaagtaaaaacgcaaggggggcgggagataggaccaaagccttggggcgcagcctgccccacccccaaattccttgaggctacttgctgtgtgtgtcagtcaacc

SEQ ID NO: 453 - DNA, 428 bp, Homo sapiens
gggacctgggaaggagcataggacagggcaaggcgggataaggaggggcaccacagcccttaaggcacgagggaacctcactgcgcatgctcctttggtgcccacctcagtgcgcatgttcactgggcgtcttcccatcggccccttcgccagtgtggggaacgcggcggagctgtgagccggcgactcgggtccctgaggtctggattctttctccgctactgagacacggcgggtaggtccacaggcagatccaactgggagttgaagtgtgagtgagagtgaagaggaaccagcaggcttccggagggttgtgtggtcagtgactcagagtgagaaggccctcgaagtcgtcgtccctctcatgcggtgccacgcccatggaccttcttgtctcgtcacggccataactagggaggaaggagggc

SEQ ID NO: 454 - DNA, 1806 bp, Homo sapiens
ctcagcaggtcctgtgcagccctggggccaggccagaaccccaggaacaagccactttcgggttctgagccttagcctcggggagctggaccaggggagcaggcagtggcggggggtgctacggcaccccaggatcaagcctgctgagggggctcgggctggtggccagcaggctcggctgaccagcaagtgggtggtcccgaggcggccaggcagcctgacccaaggcagcacgaccgtccacgggtcatcaggctctagactccggggtggagacgcggggctgcgggaccgagtcggtgccactttctcgcacccctgatcctccgtgcctcttcccaagaccgccagacccttcaccggcccctgccactgccgggaacgctcccgcctgctccttaactcacacaggcaacaagttctcgcttcctcagagcagccacggtgtgctccggaggcccggcgcaaggtggaagctccccgcgagtccgcgttgcccacgccccgaggggcgcccgctgccgtcgctgccccccaggagcgtggagctacaggtagcgccggcaagggctgccccccggggcgcccctccgaccgggacgctgcggcatccccggaacgggcaacaggtacgcgagcgcaccgaggaccgcagccccggctcctacagccccggccctcgccccttacctccagcaactgcacgtagctcgccgggaaccagccacggagcccgtcctccttctcgccttcccaccagccgccgtccgggacctgcagcagcgtgatcagctcgcccgcggcgaagcgcagcccctggccgtgccgctccccggagaaggggtacagggtccggcagcgagcgccggacatggccttggcgcccgggttcacagcgcagcctgcattcccgccgagccccacgggctgggcagcggctccgcggggtcccaggcgcccggcgctccgggctcccgcgctctgggcgcgcgccgtctctggggtgcgcgggggtcctcaggcaggcgggggacgcgcgctccgcgccgggaagcagagactcgttggcttcgcagagcgagcggcgacgcccccgggccgggcagctcgcggatcctagctctgggctggggggcggggctcagggggaggagacccaggcgccgcgggctccggagacaacttcccgggaacgcggaacgcgggctgccgccgcgagggctggccggggccggggccgggaccggggccggggccgccctggcgcgcggaaatgcgcagctcccccgggaggtgggacccggagcatcccccgcggcaggactccgcggcgggcggccgtggagaggagctggcggccgggtggccgggcgggggttagctcactggcgagggggcgggccggggcggggccagggcggccacgcccgcggagggctgcgcgctccctcgcagaggggcctgtcactggggagaggggctcccactcgcgcccccaccgccggggaccgcgggaatggaaacgcccctctgttctgtgcctgctttgtaggcctggcggcccgtggcgtgcgggcggcttcttcgaggtgtctgcgagggatcgtttgtggttgtcattgtagtggttgaacctggggttgaacttgtctcaccggaaactgaacaatgggtgaaagacaggtttgaagcctggcgcctgagcccagcagcctggtggcagcgtggcacgctggagggacggaatggagagaagacggggtcggagttgatagaattggaaacagtcgatggggataaatggag

SEQ ID NO: 455 - DNA, 2265 bp, Homo sapiens
cagcctagtgccactctatcaactgctgctgctgcggtgccaagtttacgtgggggaaatggattctgaagttttcagcagtttctgcagctcccaccaactccggggtcgtcctccctgccccctcactcaccccagccacagaaagtcaagcgtatcgttagggttgccctctttaacccttctctttttggagtggcgttcgcgcctctcctctttctcagactctagctggtccaagcaccggagctttctgggagagggcgatggagaaggggacagacagcggccggcccggggggtgtccgaacaggcaggttggtgggttaaggtcttaatcttgactcgagatctctccccggagttcacagagtaggcgacgaagccgaagcagctggagcgcgacccggaggagtctgacttctcgttgtcttcataattttcattcgttgctttcttcgtggacttgcggctgggggaggatccccgcttcccgcccccgagggagttataattatccactttgagagcagccccttcccactgaaatgtccctaaagtgggcgccctgcccctcgggcccccgtccgaatctcagggcctcacggaggtggccataccctgccgccgaggggttggggggtccgggggagggtgggggcgcaggccgtagggactgcgcgctgctgctgcctccgcccggaccggcttctccactgactgcgccgaggcccgggctctccattgttcggtcacattggaaggcccccgggaccccgaggcgggagccggccgcccaaacaacgctcaccgttacacgccgcaatcgaggcgtctcaaccatcgccagaaccaggacgattgctccgaaggccccaccacgaggaaggcagccaggcgcccgcgccaaccaagcgccttgtttgcgctccacaggggaccgcaccgccactccatccacagcgcaaacaaaccactgcgagccccagggcgcaggccggcccaacggcctccaggggcggcgcttcggccgcaagtagtgaaaagaagatcaaaacaactcaccccgcccttcccagagcctggccaagggcgcgccccaccagcgccagccccgacgggtccaagccaccagcggcggaagcacgcgggaggagcactcatgccggatctcaggcatgggccggagaggccgcagcgtagggctgggactggaggggcaggcagccgaaaaaagaggctgggcggcgcgcctcgcggggtagcaggggcggcgcgcctcgcttgcgtgacaccgggccgtccgggggcttacctggtcgccgagcaggcgggcgggtaaagctaggccgcgagagcgaggttaggagaggagaggaggccgcagtactgctcacacgctccgctcttctcccactctcgactctgcagagccgccagcagccccgcaccctttatccgcggcccggggccgcccccggtgccctgattggctcccagcggccaggagcccgggccgatcagctgatgcggtcccgggccccgcggccattgggcgaggagcccggcgggcgccgctggggtgggggcaagcgaactgggaaagaaagaaagaaagagaaaggggcgtgggggaggggcagggcggcgtgggggaagggagcggagccgggagggctgagggcagaggggcgagttgccgccaggccgggaagcctcggctcgccgcccacccgccagctcgccaggtgccgccccacggcccccaacccggggtcccgaggctcccagcaaagcccgccctggacctctgccctgccgccttcacggggaatagttttacgaggagagctccctggggaaatgcggaaccagggaaaccgaatttagccttttgttgcgagggagcctccgagccccgcttcctgcgggcaggggttagcttgagagggagcgcggggcgcgggacccgagggaactttgggcgggcctgggctagggcgggtgccgcgcgggcctggggacaggccggggttcccaaggcctggaagagccgcccgctccggctctcttgggtctggggaaataaacagaactctgccatttcacatttgaaatcaacgtgctgtttctctaattttacagagaggattctggagggcggtagaaactaagtctggagacctgtctcatactgctggccagaccctaggcaggtctttcagggcccaagtttcctcatctgcaaaataaagtggtcatcga

SEQ ID NO: 456 - DNA, 1129 bp, Homo sapiens
gcagcagcagtagaaacaacaacagcaagaacaacaatagcaacaagccaggttcaggctaaaaaagcaggcagtgtgccaggaagggggccgactgagcagtgtgtggaagctgtcgtggaggtgaccgtggggggatgagaaaaggggaaggaaggaacagacagcaccagagggacttgaacaaagggagaagggggattaaagtcaaatcactgtcagcagcccaggagcagacaaaaccaccagagtaagccctgaaatggaaatgagagggaaaagaagcaagccgctaacagctaccaagaagaggaggaagtgggagatgcgctgcttcctgtagaaagaacgttgattgaagacaaagctgggtggggactagtccctgggctgcagccgctgctacacatactcacaacgctgccgccgcgctccgtgggcaactcctactactgctgggctgggctgggctgggctgggctgcgccggagctcgcctgcacagatcagctccggagaggggaaaaccacgctcctcggaccaagcctcgggagctaagccagatctgccagtgagcctcaggctttaggaactgaagagtgtttctgaaagatctatccagcactccgtgactggtgctttcatatatttgtgacaatttttgagtaatattgcatgaaaatgtccctatgttacatccattcagaagttttgttgttttactctaaagctgggaaaggaaatgagagggaaaagaccccggagagggaagaaaatctcagtattttgaaaattgagattacttcagagccttagccacacctaaaatacctcctagttaatagtggtataaatgcctcttcaatacgttttccagaatccaaagcattttggtttatccaggacccagggcagcacagctgtcaccaagcaggagagttaaggattcaccatgagctgggaaatgcttttgccatgagtatgagcaaattccctctttccctgaatcatggacattctagattaaaagaacatttttttgtgctcttaacaagaaaaccatggccctcctttgttcaagtatcagaagaaataaacccacagctccagagaaggtgaccattctcag

SEQ ID NO: 457 - DNA, 2793 bp, Homo sapiens
acaccaactaaaaggaagcaatggatttccttttaaatggagaggaggatgtcggagagataccatgaaacgaattctcgaaactccaggcagcaataccgatgaaagcgcattcttccaaaacagagctcctcattcagggccacatctgagcctgggggcccaatgctcagagcgtgcctggctttctggggaaaagcgccaggaacatgaacgagctacgaagagctggagcgggcgctgcgggagcagcgtgtggggaacggctcgggagtcgctgctggtaaccccggcccagccgcccgcagctgggctcgcagccggctcgcagccgacacccagggtgctgcgcccggggccccgggccgcggaaggatatttcactttgtttacctccccggccgggcgagggagctgcgaggccggtgctggcgcccccggcacggagctgcaatggcagcggcgcggggagccgcccgaggactggcagcggcggggctgattgatgggcgcccggcgcaatgaggggggcccgggcgggggcggggcggggccggctctgggctgagccagtcgcgggggcggcggggggggggcggaggctccgtggaaatgtgtcggtgacattgaatggggagaccgagcgcgagcgaggcgcgcgcgcgcacacgcgcactcacggccccgctcgctccgagatccccggccacgtaagtaactattaataccgcctcgccgggggatgcgggcgtgctgagcgcggggattggcctcgggcaccgtcggccgtcccctttaatttttaaatacacggtcccctcttttctctggggggggcaagcaagaaatcaaagaaggaggagacaagccgtcaattttctccaaaacaaaccccaccgggcaatttggtctcggggtagggggagacggggtgattgcaaattattccaggacgagatccagttctccagcgggaaaggggcaaaggaacgccgcgcgttggaagggccagggacgcagctccccttgcagcgcccgcaggacccccgcaagctcgtgccggcgaaatcggagaccgccgatctgtcctcgttctctcctgcacgtctggctgcattcggaggaagacctggggcgcgagcgagcggcgacagcatgagcctgtgctgacctccgcgcggcgggccgagcccagggctttgtcgcggtacctgcgcccagcccgcgccgcaactctgtgcccagcttttgcaatcttttgttggcagcgctgaccgcaccaagttaaatgctcccttgcaatttttcttttttttgtttgtttgtttaatttttggagagctcgcgatcttggaaaagcctcagacgccatctacagttaaaacgtaggtaactgccctctcccgcaccccccccttacacgccccccaccctttccaccaaaaaaagggggtgcagcgcggattctggctgccgtgcgtcgccagccggtagacccgtgcttgtttcctttctctttttgtttggcttctaacgcgttgggactgagtcgccgccgtgagctccccgaagactgcacaaactaccgcgggctcctccgccccgtctgcgattcggaagccggcctgggggtcgcgtcgggagccctggcgctgcagctccgcaccttagcagcccgggtactcatccagatccacgccggggacacacacacagagtaactaaaagtgcggcgattctgcacatcgccgactgctttggggtaacaaaaagacccgagttgcctgccgaccgaggacccccgggagccgggctcggagcagacgaggtatccggcggcgcccatttgggggcttctaactctttctccacgcagcccctcttctgtcccctcccctctcgctcccttttaaaatcagtggcaccgaggcgcctgcagccgcactcgccagcgactcatctctccagcgggtttttttttgtttgtcgtgtgcgatcctcacactcatgaacatacacaggtctacccccatcacaatagcgagatatgggagatcgcggaacaaaacccaggatttcgaagagttgtcgtctataaggtccgcggagcccagccagagtttcagcccgaacctcggctccccgagcccgcccgagactccgaacttgtcgcattgcgtttcttgtatcgggaaatacttattgttggaacctctggagggagaccacgtttttcgtgccgtgcatctgcacagcggagaggagctggtgtgcaaggtaaagggccagtgggttgctttttgtctttggaaggggcccgagggagcgggagggcgccaggccctcgagtctgggagagggagattcgcgggataattaccgtggccttattaaatgggtttatttatttatttgctcaggttcggtaagttgcgaagtttttagaccgtttcagacaatggggcgggcggcagtgggggcgtttcggggagagcccggggaggagagggcggcgggactgcgcgggggccacggacacgcgtgcaccgaaggctccaggagctctctgcgcgaggccgggtcccgctgcccgggggggatttcttcctgtgtctagccccctccccttccaacaaggattagggaatcccccggtaattttaagactgatgacttcgttcttttcgcagccattgttcttagcagcgggcaggtgttaaacctttgttccgaaggtgccctttaaaacagacacacaaaggtgcccccttcggctgagcccaggggcccagc

SEQ ID NO: 458 - DNA, 400 bp, Homo sapiens
cggcgcgccagttcgctgcgcacacttcgctgcggtcctcttcctgctgtctgtttactccctaggccccgctggggacctgggaaagagggaaaggcttccccggccagctgcgcggcgactccggggactccagggcgcccctctgcggccgacgcccggggtgcagcggccgccggggctggggccggcgggagtccgcgggaccctccagaagagcggccggcgccgtgactcagcactggggcggagcggggcgggaccacccttataaggctcggaggccgcgaggccttcgctggagtttcgccgccgcagtcttcgccaccagtgagtacgcgcggcccgcgtccccggggatggggctcagagctcccagcatggggccaacccgcagcat

SEQ ID NO: 459 - DNA, 1394 bp, Homo sapiens
cgggcgtgatctcaccatggtgcgttttggctgctactttctttttcagtctttcggtgcggagaaggggaggaggcgggcagaggtctgaaaaaatcgaatgccttatggaaaggaactgcaagggttcctttggggtgatcaaagagggagacacagacacagagagacaaaggcaaggaggactgtctgggagccacgcgggcgatacagtttccgaggcacgccgcgtcccgcctagcctgttgaacaggtagacatgagcgacccaagcgtaagtgccgctgccgcgagtgcccagggtgcactcgcgcggggaccgacaggtgccactgcagggtcccctctcccgctcactgcctgccacccctgacgcctgctctctttctccattcgcagtgcggatttgcgaggcgcgccctggagctgctagagatccggaagcacagccccgaggtgtgcgaagccaccaagtaagtggtcgctgcatccccacgcgggtcggggttcttcagacaggcggtccctcttccgggacccccgcttgggctgggtctccttcctctcctgagaaaccttcggtacgctcccagttgcccagccctgggaagccagcatcgggttcccacgctggggaggagcgagaggaaaggagaggacgcccgaggggccggcccagggcccgggggacccgcggcgcccgagggaggagcacagacgggggtgggcgccgagcagcgccgcgccggctgctgagctcgcggggccgggccgtgagaacgcgcggggcgcgccggggccgctggtctcccacggccagggcgcctgggccccagagcccatcagccgcgtgcagcgctgcggcctccggcgcgccggcaggcaagcgagcgggcgcagctgggggatgcctggaccccggaggcagcagggctggaggcggtggcgggcttcagggcaacttcacccggccggctgcggcgtgggtaagaggcgcgtcctcctccacagcaaaagctgcgatcgctgggctgcccagagaaggcagccggctaactgcccagcggctccacgaggtgtgcgcgtgtgtgcgagagagacaaatgatttactgcttagcagtcccatttctgttttcgttaggactgcggctcttggagaaagcgtgagcagggggccaccgcggtctccgcgcctgtctgcaccctgtcgcctgagctgcctgacagtgacaatggtgtgaggggctgcgttgcaatgtggtcatgtgatttcaggctatggatttcgtgcgtctttctctgctcactgattttcacatggttattctttcattttgcactcgataaacagtttacatcttctgaagtaagccagaagattgtatggctcac

SEQ ID NO: 460 - DNA, 874 bp, Homo sapiens
ctgtatccggtagtggcagatggaaagagaaacggttagaaaagcgtggttcttgcgcaggaaagttggagcaaagaagagttcagaagtgggcgggtgtgtgtttaaaaaaaaaaaagggggtggaaaccccaccagccaagtctgcagaaaaaaaataaatgaagtctgcctatctccgggccagagcccctcccctcggcccgcgcgggaggagtgtgacccaggtgccgcttcctctcgccgccgagggtcaggagcccgggagcgcgaccctcccccggcccggcctggcccggcctggccagtccccgcggtctctgcccgggctgacgcccaggaatgtggtcgacgagaagccccaacagcacggcgtggcctctcagcctcggtgagtacccgccgtggggaagggtcctggggacccactggaggccgcggcccgcagcagccaggggccgagccacggccacggacgccctggtgtcccggtccgtgccgggcctccaggcggaggaggcgtccgctgggctcagatccccgactccagccccggttccccggcgcctgggctgcgcggagtccctgtcccgcgtcccggacccctacgcgcagcctccacgcgctccgagctggagaagccgccagcccgccctcccagggcgtgtcgcccgctttctgtttctttgtgcggggcatgtgaaatgtgtggggccagaattgtctccctaaagaacagttaggtgagagttcacatcacaagttgcttctgtgctgctccccagccagggtccgaccggccaggctccggtgaactccaggcaactccagccttgcccagggacttgctcgctccgctgggcgccgcaaaggc

SEQ ID NO: 461 - DNA, 1416 bp, Homo sapiens
atcaggctcctcttggccacgcatcctgccatctcctccgagcccgcctctgcccttctccccaggccagctggggtaccctggggtccccgaccttcctgggatcgcccagatctgcacgtcaatgcccccgctcactacaccctcgccccgtcttcaactggggcctgcaagactcctactcccttcaaagcccctcccggacaccgcgtccctcagggtgccccagggatgccccggggtcccccgtcctcacctgagtccgctcgaagctctcgcgctccgccgcctccagcccagcgccgaccccgcgccggctcagcaccttgggcagtgggttgggcacctgcttcatggagctggccatccgatccatgtcgccgcgaggagccgagtcaggggccctcggctgccccgggatccccacggcgccgcccgccccgccctacccgcggggatccggcgctaacgggacggctcccgcctgccattggctgtgagttctgccagtcttccagagaccccgcccctcgccgaacggcttccccgcccgcccccgccccttcaggagcccctccgctggctcggccccgactagcctctcggggcctcgaggcgcgtggtgactggtctggagtcctgtcagtcaccgtggtcccgccgttctaggcagggccggggcggggtgcggacggggcgaggcctggatggggcgtggcctgggcgggctgccctgatatgccctcgctcgccccgcccccgcccgcgcaccgccctctcctcccctcggccgcagtccccgcgcgccccgagcgtgctgccctccgccaagcgccgcccactaccctgcccgctcctgcagggggctatcccgcgacggcccgtggagatgggcggggatgacgcgggccgggcagtggggcctcccccgggggaatcccagcccgcctgcgaatagcccgttagctccttccgggcctgggacccgggagcgccggactgaccagccctgcggccgcagcccgggaaagcgcagccccgcgggcggggcgcagcgtgggcgcctggccggatccggctcaatgggtggccgcccccacccagctcccaggcttccccgtcctgcacttctccattctgcccctgcccatgccctctgcaaggatcgcagagcccaggtcccggagttcggggctgagtccgcgggcacctcccgggacctgcctcccaaacctcatcaaaggaacccgtcagagcttatttgttggtagaaagccctgtagacactttcggtttatcttaggctctgagccgcgcgtcccatcctgctcagacgtcagccagggtaccagcaagcagagagaagtgtgatgcttagcaggagtgctggagaaagttaaaagttttccgttaaac

SEQ ID NO: 462 - DNA, 498 bp, Homo sapiens
ttttcctcttctccccctttctccctccctctctcttctgtccgcctccccttctcccctcagcctctcgcccccctcccagtgtccagcccagagtctgcgccccgggcccattgttagcaggctattccacggcagctttgcatctggctccggcgggaagcggaaaacgggaggcggctctcgaagcttcccgaccttcctgcgccatcaacttctcaggagtggctggagaaagatgcatgtgccaagcgagacaacaaccccaggtccatgtgtccaaatccccgtagccagggcggcggccagccaaagaaatgccgccccgagcaggcgcgtgcggctcctggcattctgggtttcatacccgtagggctcgggtgcggtgagtatttccgatttccaggaagtctgttggaagttaccagcagggaaagagaagatgcttgctcctctctttccctccctctctcttttttttctacccttcttcttgtc

SEQ ID NO: 463 - DNA, 2180 bp, Homo sapiens
tccctcaagtttctccaggagccagggatagatagctgggcgattccgaggtccaggggaagggaaatggcccttctctggctctcagctcagggccccccgcttccaggccggatttgccttttcttcttcccgcaacgaagattcccgcccctcagcaactttgaaaaaagcatgggggatcgtaaactcgaacttcgccggttaatgggcttatttattggcgctggcggctgcttattttggatgccttacaaacatccgcgctatctgcgggcgagctactttccctccctccccctccccccgcgtgggccgcgccgcgcaggctgggcagggaccagggctctgggtcctcccggccacaggaaagagcgcacaggagggggcctgctcgctggtgtcctcgtccctagtcagggggagctgaggccagcgccgaggacgtcttgctgtggggcgctaagccggacatgaattttactgcgtccccacgcccaaatattaaaaagcaagttcacaaggtcagcctgcctgcagcttgggccaaggccggccggctgctgcgcgggctcctagttttctgatccttctcctccttgtgtctgcctgtctgcccgcctgactgcagccctctgcagccctgcttacccagggaatccttctccggcgaggctttgctgctctcggaaggggccggggagagctcctccgcggccgaggacgacgcgtgcgcctcctcgtcgccctgcgagcccccgccgctgccgcaagccagcgtggggggcggcggcgaatcgagggctcgctccttccgggccgcatcggccgagccggaggctagcgcgggcgggagatcgaaaccgcgccccgggggctgcgcggggaacgggccagccccgagttgctgcgcgccgccgccgccgctgccatagcccttggcggtgccgtaggcctgagaaaggcggaagtagccaggcactggcaccccgctggaggtgcccagggcgcagccgtcgggcggcgggccccgcgggaagggagccagttcggcggcggtggccgagactttggggcatttgtccgccgagtcgtagaggcagtaggagctctcttctttgatgttctgcgcgaaagagcacgaggtggcctgcggcgctggctggggtggttgcggcgggggcggcggctgctgctggggcggcggcggcggcccgtcaggcggctccatccggcaagaccggggcgcgtctagccacaggtctatgggcgagggcccgtagccgtgcgccccgggacctagacccccgccaccgccaccgctgcccggcgacgctgcctcattgcgcttgccgcccagcgtggggaagagcccgcagctctgcagcccgtagggcaggtcggcggcgggcggcaggtagaccccgccgtgggcgtagtaaccgccaccgccgccgccccccgcgccaccaccaccgccgcctgcctcgcctctgcccgagctgatgagcgagtcgaccaaaaaagagttcgcggcggggctctccgagcatgacattgttgtgggataatttggcgaagggagcagatagccctttctggctgacatttcttgtgcaaaacatgctgaatacgattagcaatccccccgcaccgcggcgggcgcccgcagccaatcccgagccagagtttccgcgcgaccactcccagtttggtttcgtaggcgcggggccgctctccgagggcgccctcagagcccgcgattgatataaatatgtaatctgtattgatgggccaggagacgcaccccgacaccttggcccgaaggccgggagctgtgggggctgccccaacgtggctggtggggggcctggccattgggctcgccccgcccctacccggacgtgagccccataccggggtcccttagaagggcccttgggccccgcgcagttaacaagtggggtgtttatggtgcgcgcccagtctgccttgggtgctcaccatccctgtcgcagaagctgccactagtccccggtgtactctaaccactgaagcggccgtgtcggggactcacgcgcttcccattcagctctggatctggaactggccccttgtctgaattctgcctcctcaaaagtggcgaacctggccctatggccgtcaggatcctcagagtg

SEQ ID NO: 464 - DNA, 2379 bp, Homo sapiens
ccagggccctgattgcccaagactcgataaggggagaaagaagggcatcattggtccaatggggaaggcaggaaaaaccgattcgggggtcaagggccctccctcaagtttctccaggagccagggatagatagctgggcgattccgaggtccaggggaagggaaatggcccttctctggctctcagctcagggccccccgcttccaggccggatttgccttttcttcttcccgcaacgaagattcccgcccctcagcaactttgaaaaaagcatgggggatcgtaaactcgaacttcgccggttaatgggcttatttattggcgctggcggctgcttattttggatgccttacaaacatccgcgctatctgcgggcgagctactttccctccctccccctccccccgcgtgggccgcgccgcgcaggctgggcagggaccagggctctgggtcctcccggccacaggaaagagcgcacaggagggggcctgctcgctggtgtcctcgtccctagtcagggggagctgaggccagcgccgaggacgtcttgctgtggggcgctaagccggacatgaattttactgcgtccccacgcccaaatattaaaaagcaagttcacaaggtcagcctgcctgcagcttgggccaaggccggccggctgctgcgcgggctcctagttttctgatccttctcctccttgtgtctgcctgtctgcccgcctgactgcagccctctgcagccctgcttacccagggaatccttctccggcgaggctttgctgctctcggaaggggccggggagagctcctccgcggccgaggacgacgcgtgcgcctcctcgtcgccctgcgagcccccgccgctgccgcaagccagcgtggggggcggcggcgaatcgagggctcgctccttccgggccgcatcggccgagccggaggctagcgcgggcgggagatcgaaaccgcgccccgggggctgcgcggggaacgggccagccccgagttgctgcgcgccgccgccgccgctgccatagcccttggcggtgccgtaggcctgagaaaggcggaagtagccaggcactggcaccccgctggaggtgcccagggcgcagccgtcgggcggcgggccccgcgggaagggagccagttcggcggcggtggccgagactttggggcatttgtccgccgagtcgtagaggcagtaggagctctcttctttgatgttctgcgcgaaagagcacgaggtggcctgcggcgctggctggggtggttgcggcgggggcggcggctgctgctggggcggcggcggcggcccgtcaggcggctccatccggcaagaccggggcgcgtctagccacaggtctatgggcgagggcccgtagccgtgcgccccgggacctagacccccgccaccgccaccgctgcccggcgacgctgcctcattgcgcttgccgcccagcgtggggaagagcccgcagctctgcagcccgtagggcaggtcggcggcgggcggcaggtagaccccgccgtgggcgtagtaaccgccaccgccgccgccccccgcgccaccaccaccgccgcctgcctcgcctctgcccgagctgatgagcgagtcgaccaaaaaagagttcgcggcggggctctccgagcatgacattgttgtgggataatttggcgaagggagcagatagccctttctggctgacatttcttgtgcaaaacatgctgaatacgattagcaatccccccgcaccgcggcgggcgcccgcagccaatcccgagccagagtttccgcgcgaccactcccagtttggtttcgtaggcgcggggccgctctccgagggcgccctcagagcccgcgattgatataaatatgtaatctgtattgatgggccaggagacgcaccccgacaccttggcccgaaggccgggagctgtgggggctgccccaacgtggctggtggggggcctggccattgggctcgccccgcccctacccggacgtgagccccataccggggtcccttagaagggcccttgggccccgcgcagttaacaagtggggtgtttatggtgcgcgcccagtctgccttgggtgctcaccatccctgtcgcagaagctgccactagtccccggtgtactctaaccactgaagcggccgtgtcggggactcacgcgcttcccattcagctctggatctggaactggccccttgtctgaattctgcctcctcaaaagtggcgaacctggccctatggccgtcaggatcctcagagtgtcaggagcccagagtgaactagaagctgacttgcctctacttccagtatccacagatttttccccaaaatgcagtggttgttccctagcccctaaccccc

SEQ ID NO: 465 - DNA, 779 bp, Homo sapiens
gtccgggccttttccccccccctttccttttttttttttcctcctctcccctccctcccggcttcctttctttgtagccacctcaggggaagcaacagatcgtcactcggtgttctcaccgaaagcacgtaatcgccggtgtaactcatgttggctggggggcctcccggcgcgcgcggagaggctggggtgcgcccccatgcagcatgcttgtgctcaattgcagggtcctcgttctcgagtgtgcagagggcggtgagagctcaactctcgtccccacctcccacccgcagctccccgggtgggtgagggatgccctggactggggatagccaggtgggagtccgtcgctgtgtggcctgtggtctcggagtctgttctcctggagtctcgcatttgcacccccttcttcgcagtccccctcccatagacttgctctgggaagcgcctctgcctccgaccctagccggaaccccttcggggccagagtttgaagccgtggatgtgcctgcctggtggcttgtccgatttgcacggtgacttgattacactctctcattcatggtcacttccgaagcgctttagtgccttccgtccctaaaccgccaacagccagaacggcttctccccgcggtttgtcactgatccgcagggcccggaagggccttcgtcttacccgggatccacctctcccctcatcttccctgcctacctcttcatcccaccttctgtccttggagaaactccctcctcctcgctgcctgccgggcttcggagtg

SEQ ID NO: 466 - DNA, 588 bp, Homo sapiens
tgaggtgcgggctgaggcggctgtggggcagggggcgcggcctggggcggcggcgggtgcaggggcggctctcccaggcttggaggctggctaggtggggcgctcagggtgcgcaggcacgcctcactcagttcgtgtgccttggggtggcccccggcgctggagggagactggagggagcaggcgggtcggtggtactcgccgtcggcgcccaaagcggcggacgccgggtacggctgctgattggcattataagcgaacccgttggctgcctggtaggggtagccaccgtagatcgccgagctgtcgtagtaggtcgctttttgcatcgcgttgtttcacgatcttgatcgcacactctgacaggggtttgacacccgtgagggcgcacattggcacgcccccgcggtcacgtgacactccgccgccaatggccgccccgcgcagacctggtggggcgagaagcgcagcgcggtgagggctccgcgcaaatccatcttactctcaatagctaagtgacatgaaagccataaaagaaaaagtggtcagcaatatttagcagcacgacttggccccgggcgcagggag

SEQ ID NO: 467 - DNA, 1266 bp, Homo sapiens
atttatgggggctataattactgccctaacagtttggcgtctcgtaaatctcctgataaagggaccctgggtacaaaagggttctcatgttgggatcaggcggctggctggcgcgcacatacccacatctcaccgcagcccgggtcagatgggggctcccctcccgaggcccccttcccctgagcctctccctcctgaccccgaccctcgaacccaggcccagccccggcccacctcccgcgcctcccaagcggcgccacgtaccggcgctgacatggatcttcttcatccaggggtacaccacgggctccttgcccttcaggcccagcgggctcttgtcggccaagagcagcgggcacgcgggggcgctgccccctgccgggacgcctggggtggcgggggccgcctcgcagcgccgcggggccgctgggggcacggcgcgaggctgcaggggcggcggcagctggggctgcaggacgtggctcgcatgcaggccgtgcgctgggcccttggcttgcgccgggggctgctcgggctggggcggccgcccggggctggcgccgccgcggtagccataggggtaggcggtgtccgcggccccatgcgcggggtacagcgcggcagcagggtaggcgggctcgcgggcggtccgcggcgcgtagtaggaggcagtgggctctcggccgccgcccgcgtgagggagctggggctgctgcagcggcaggtgctgggtcgggggcgctgggggctgctggtagccggggcccccgcccgggccgccgtctgcgccgcccgagccgctgtgctgcgcgtactcctcgaagggagggaacttgggctcgatgtagttggagtttatcaaaaacgagctcatggtcattaatttgtgaagtgcaaaaatactaatttttctcgcgttgtcgttttttctgggcttgccgaggcccctccccctcctgcctcgcttcccatccccctttcctctgcgcccttcccctccccccgctgtcaagtgcccactcctccccctcccgcagacgccgccaccaaagttcgagccgctcctccccagcccagcgcgcgccccgccccgtgccccacgtgcagcgcccccaccaatgggcgcaccgcgcgcgcggacccggatcaggaaacgcgcgggtgcgtgatggatgctgctgtccggcccctgggctgggggagggagcaggagctttggaccccagccccccagctttggttcccgctgggaattcaggccctgtc

SEQ ID NO: 468 - DNA, 1166 bp, Homo sapiens
caagacaaatggagttaaataaatttacgaggatcgaacccattaattgggccataaaaagttttatgagcctcatttacatacaatgctatgggctccacgcaatggcgcctccgctccaattaaaaccagaaaggctgcgccgggagtcacggggctaccggctcgcaacagcctggctccgctcttccggccccgcgccccgcgctccgcgctccccagcgctgcgctccccgctcccggtcccgctccgccagcctggcccgcctagcgactgcgcctacctgaagaccgcatccaggggtagatgcggaaattggcctcagccgcgccatgcagcgcgccctcgtccgtcttgtcgcaggcgcctttggcgaggtcactgcagagcccggggatgttttggtcgtaggaggcgcagggcaggttgccgtaggcgtcggcgcccaggccgtagccggacgcaaaggggctctgataaagggggctgttgacattgtataagcccggaacggtcgaggcgaaggcgccggcgcccgccccgtagccgcttctctgtgagttgggagcaaaggagcaagaagtcggctcggcattttggaacagagaagcccccgccgtatatttgctaaaaagcgcgttcacataatacgaagaactcataattttgacctgtgatttgttgtccggcagctttcagtgtcggttttacgaggtagagtgatatatgataacattacacccccagatttacaccaaaccccattttcttttggacggagctcgccgcagcacgtgaccgcccacatgaccgcctccgccaatctcagcagtcctcacaggtggtctcgctccgcagggcccgcagccgcctagaatggaagggcaagaggctcaaatatgcggccaaagaatccgcccgcgcccggcgggcctggcgcgtcccgcggaaaaagacctggaggctccgcgggagcgcccagctggcggccaacctccgcactggggtctgcggacgccaggcggcccggccccacgcagcaccccccaccccgcccccccgccgactcctgctagtgagccctggaccaagcttgggatcctccccatccctctcctgtccgcctgcccagaccctggaagggtctctgtcccccgcaacagcc

SEQ ID NO: 469 - DNA, 2747 bp, Homo sapiens
aagattaggattcctctgtcccgttcactgacttcgtctttctttcccaacctgtccctctacgccccccactccttatttaaccttcctggaaggccttcggagctgggcaagccgtcagggcgccctaaggccgctgatcacgtctgtggcttatttgaataatctgtcatggggacccttgtggcccgggtcgcccgcagcctcatcttggcaggatttacgccgccactggccgaaggcaagaagtggaaggaatcggccgtctcccccagcgtcccagctccggctgccctggctgccgccgctcacggacaatctagttgtacaaaaggctctctgggctgcactgctttcgaagaacggcccaaagtatctcggtcctgggcctgggcagccaaggagaggggcggccagtcttggctcgtcccgaagtgcccgccccgccccctctcgctgcagcagccgcctcctctcccgtagccctgcgggccgctcttcactgctctccagacttggggccctatctgaggcgtcccaaacaccaacttctggctcctggccccaactcgagaggcttccagcgaggacgaaggcaggctcgagagaaacctggcgggccagcagatccgggaggccggcgtggaggcggcggcggatttgaagggaggagacacttactgggatcgatggggggcttgtctccgccgctctcattctcagcattgttttcagagaaggcgccttcgctgggttgtttttctctatcaactggaggagaaccacaagcatagtcagtcagggacaaagtgtgagtgtcaagcgtgggacagtcaccccttctggccgacagcggttcaggtttaatgccataaggccggctggagggcaagcccgcgaaggagagcgcaccgggcgtgggctccagccaggagcgcatgtacctgccgtccggcgccgccgccgccacgggcgcctgggggtgcacgtaggggtggtggtgatggtggtggtacaccgcagcgggtacagcgttggcgcccgccgcgtgcactgggttccacgaggcgccaaacaccgtcgccttggactggaagctgcacgggctgaagtcggggtgctcggccagcgtcgccgcctgccggggaggctggcccagggtccccggcgcatagcggccaacgctcagctcatccgcggcgtcggcgcccagcaggaacgagtccacgtagtagttgcccagggccccagtggtggccatcaccgtgcccagcgcctggcccgcccggcccgacccacggaaattatgaaactgcagatttcatgtaacaacttggtggcaccgggggggaagtacagtcacctaataagttgccggcgcccgcgcccccattggccgtgcgcgtcacgtgcccgtccagcagaacaataacgcgtaaatcactccgcacgctattaatggtccgatgttttgcagtcataatttttatagcaaaagccatatgtttttatgtaaagggatcgtgccgctctacgatggggtttgttttaattgtggccaacgacgattaaaagatcaaatctagccttgtctctgtactctcccgtctccccccccatacacacacttcttaagcggactattttatatcacaattaatcacgccatcaagaaggcgcgggtcccgcgtgcgagtgcggccagcggagcccctcacataaaattagacaataattgaagccataaaaaagcagccaaatcgcattgtcgctctactgtatttaaatctatatttatgatatttcataaggagttattgtttcagaagccacacaggctggcgggaagtcggaaacgaccaacagattcgtttgcctcgccgtggctcccagctgtaaaaatttacgaggacttggaaaggttagactgttgtgtttggttggcgagctccctgtaaataatccctgcggtccccgggagaggcgagtttacccgcggccgccctcgaaaagtcaaattcaacgcaggatccgtcccaaacggagccgccgccggccctaccagggcactccaggcagggaccggccgctcagggagtaccgcgggtgtaggtccccacagctacccgcctggagcgaggggcgcccgggcaacccttaaattcgcctttgctacgaggaccccacggaggagctggccaggagggagcggccagccgccaccagggcgaaggttttgagggcctggttggttgtgcggcgcgctcggtccccggccctcgaccccacgcacacgcgcgcccagcccgcctttctcatcagctggcaatcaggattcccaggcgcaggcggctggcgacccagccctgtgctccagcctcagaggctctaaccatgagcgctgcaagcctggttgcgctccgtgaatcccagctggggaaaaaactacaagtggcatgaatggaaggcaagttcggtttgggaaaaggcagcctcgcctaagagaccccgcagctccggaacctgggaggcccgcaccgatgtggcctgtcccggggccgcgtgagcctttcagggctccttcctccctttccagctgctactccgggcctcgccttggttacctacggggcccggagactcggcggagaggtacaaggcccaaagagaggcagccacagctcaaggccagggctggaaattagaacggggaggggtaaaagggcatcgactccagtcccattcct

SEQ ID NO: 470 - DNA, 521 bp, Homo sapiens
gggaggctggcccagggtccccggcgcatagcggccaacgctcagctcatccgcggcgtcggcgcccagcaggaacgagtccacgtagtagttgcccagggccccagtggtggccatcaccgtgcccagcgcctggcccgcccggcccgacccacggaaattatgaaactgcagatttcatgtaacaacttggtggcaccgggggggaagtacagtcacctaataagttgccggcgcccgcgcccccattggccgtgcgcgtcacgtgcccgtccagcagaacaataacgcgtaaatcactccgcacgctattaatggtccgatgttttgcagtcataatttttatagcaaaagccatatgtttttatgtaaagggatcgtgccgctctacgatggggtttgttttaattgtggccaacgacgattaaaagatcaaatctagccttgtctctgtactctcccgtctccccccccatacacacacttcttaagcggactattttatatcacaattaatcacg

SEQ ID NO: 471 - DNA, 1405 bp, Homo sapiens
gtccccctagagtttaattattcctgagatttcattggaaggagtctaccaaacggaatttttctgtgtgaattttaaaagataaccgagtgcccaatattttagaagaagaagaaagggagtggattaaacgctaattcagtaatacctgaattttagcaaaacacataagtctatgcgactgagggtgggagaggctcgatttttccagtagacggccaaggagcgcgggggtcgaaaggaccgggaggaggaaacaggttagggaaactgcaggtcgatggcacagagcgtactggtgaaaaaatccagctcttcctcggaaaaagggaccgggctgtcgagagaaccctgtaggctaggggagaggcctccggatagctggagacaggagtcggccgcgaagaagttgaggtcgggaaggaaaggtgaatcctggcgccccgagaagacgtcttctggcaatggcccgggctccagcccgccggccccgcgcagcgcgcagccgggactcgacgcgcctgcgccctctaagcgaacggctaaaggccgggggtccgcgcttaaggcccccggcaccacctccggcgggtgacagcaggcttcccacgccgcccgcgaggcggaggggccgcccgggctggccgcgggttcctcggcagggtcgcagatgtcctccagggctcccgggcaggcaggctccccatccggcggctctcggtgctgcgtctgccgcttgtgcttcatgcgccggttctgaaaccagactttgacctgcctttcggtgaggtccagcaaggccgcgatctcgacgcggcgtggccggcacaggtacttattaaagtggaattccttctccagttccagcagctgcgtgttggtgtaagccgtgcgcagcctgcgcgccccgccgccaccagcctccggcagtcccaggccatctgcagggaaaacaggctcggcgtcatagcgggccgtgtactcagcccaacctcagttcggactaccccatcctccaaaaccagtatcacctctccccttcctcgccaccccacccccgcccccaccccaaagccgctcgctttactgcttttgggttttcttctctccctctctagtctacagccccggcccgggtcagggcaaagcattcagagccgcccacgggccacgcagggggcggagagcggctcccttcggcgccggaacctcagatccccctcccaagccctcccagtggacccctcaattcggaactttccaactcaaccggagcgagggtcagaaagattgttcttcacccctcttcctccctagctctgaattcttcctcttctcctagagttggggcagaaaggaccgaatcttttctgcttctagggtctatcccagcaacaacatactc

SEQ ID NO: 472 - DNA, 1405 bp, Homo sapiens; identical to SEQ ID NO: 471

SEQ ID NO: 473 - DNA, 1335 bp, Homo sapiens
gctctgtctacgaagctatgaggcccctcttagatacttctcccccttagaaaaatgaatctatatttagggtaaagccaagctctccttgtccccccgatcccaccccaaaacgcagagaaggaaagccgagaagacaaaaaagagagagagaattaccatggctgatgtgaagcttcctcatccaggggaatatttgcggagtctgcccctcgggcgcggctgtggaggtggccatgggctctggctgcgcccgagctaggctggggctgcttagctggcttgccgcttcctcaggctccgaggacgcgctggcctcgtctatttcggtgaaattggcgctggagctggctgaggtcgcctggtcggagggggacgaagcagagggcttggcgccgtggctgtcgccgttggtgcagggcagggactcgggcgaggacagggagcagctcgaagccgcttgcctgaagcggggctcctgggcgggcgcggggaaggcgcgcgagctctcgcccaccgccccaaagtggctggaggaggccgaggagcggttgacgctgaggtccatcccattgtaattgtagccgtaagagccggtgtgcatggcagcgggatccctgtaagagccgctcagagagctgccactgccataatttagcaactgatagtccgggccatttggataacgccccgagaaggagtttacaaagtacgagctcatttgggtgattttggagggcttgatttgtggatcgtggtcgttataaccctgtgcttcacgatttatgatgtattaatgaattatagcgatgcactgtacttcgttttgctatgtatgcggccaaatatggggggggggttggaaggggggtggggggcgttagggaatcacgtgcttttgttgaccagtcgtaaattctcgctgatgacctcaagaggtaattcatgctctatggttacaaataatgacgatccgagaatcgttagggccgattcaatgcgagcctccgagagagggggaaaaaaggagaagggggagaaacagagagaaggttggctttggcctctgagcagagcgcgccacctcccggcgaccgacgggagcgcgggcgtgagaccgccatggcggcgggcggctgcctccctgctggctccctgcaggctccctgcagtcgccgggagagaaagaaacccggccaaggctcagccacagggttgagccttttagagcaggggttgggctttttacccactttcttgtgggtggggttagcctctctatttggttctttctccctacatcatcattgtcgttattattttaactttggatga

SEQ ID NO: 474 - DNA, 999 bp, Homo sapiens
gggtggggttcggaacctcacaccctcacacctagttcctagcttctagggagcctggagccggggcttccccccctctgctcgctgcttctctcccttcccccttcctccccctcttccccctccttcctcctccccgccggcctcggtccgcgtacttaaagcggcgcgggagggcggacgcgggcgggcggcccgttcgggcggtggcagatgcgcccagcggtgacagcggccagcggcgcgcaggtgaccggcctgaggcgcagcctggtcagggagcgcccggggagagctggcggcagagggcagccgatccgcccccagcgcgcgcgtctcggcgccaggagccgtcccggggcgtgttggcgagcgttgatatagatataaggacatttctcttcatggcgtcacgtgacataattaccaccagaatcaatcaagatgaattgcacgtcagcgcccggtggggatttttgcttagttgatcctggcccaagcctcttgtgcaatcgatggctcaggttggctgcgcggggagcggccagaggctcgctggcgcgcacgccgcggagtcatgaacgactttgacgagtgcggccagagcgcagccagcatgtacctgccgggctgcgcctactatgtggccccgtctgacttcgctagcaagccttcgttcctttcccaaccgtcgtcctgccagatgactttcccctactcttccaacctggctccgcacgtccagcccgtgcgcgaagtggccttccgcgactacggcctggagcgcgccaagtggccgtaccgcggcggcggcggcggcggcagcgcggggggcggcagcagcgggggcggccccggcgggggcggcggcggcgcggggggctacgctccctactacgcggcggcggcggcggcggctgcggcggccgcggcggccgaggaggcggccatgcaacgcgagcttctcccgcccgcgggccgccggccggacgtgctctt

SEQ ID NO: 475 - DNA, 1625 bp, Homo sapiens
aagggtctgctccaatgcctcttacctgtgtgaaatgcctttgccgggtaccagtgcacaaggtagggcaaattagctcactcggatttggggtctagaagtcgactaactgagggattcagcaacaggataaaaaaatgggcctgttttcacatcattctgatcatctctgtccttcgtcttcattttgctgtgcaactcggggagccgaggagaggtggcaaaaacagcggttgccgagacaaggcgcaggccttggcgcccgcctcagtcgcagacagggcctgggatgggccgtcgcgcaatcaactcgtgggggtggctgcagcgcgtacgcctgggtcgggggggagggcgggaatgggaggtggaccctgcaaggggcaggagaggggtgggggccggagtgggtgggtccagccaggcctgggccgggagccaggctcccccgcgttcctacccccacgtggccgcgcgcagccaatggcacgcccccggcgggggccctcggggcgggaggcggccccccgaccggcccaggccccctcccaacctgaacttcgtttttataaacgtcccgcgatgagctaacctgttggagggcaggcgggccggaggcgggaggctcacagagggagagagggctagaggaagagggcgggagcgagcgaaccagagagaaaggagaggagggaggaggcgcgccgcgccatggtgtcctgcgcggggccagggccagggccggggccgggccaggccgggccatgagccgcgccgggagctgggacatggacgggctgcgggcagacggcgggggcgccggtggcgccccggcctcttcctcctcctcatcggtggcggcggcggcggcgtcaggccagtgccgcggctttctctccgcgcctgtgttcgccgggacgcattcggggcgggcggcggcggcggcagcggcggctgcggcggcggcggcggcagcctccggctttgcgtaccccgggacctctgagcgcacgggctcttcctcgtcgtcgtcctcttctgccgttgtagcggcgcgcccggaggctcccccagccaaagagtgcccagcacccacgcctgcagcggccgctgcagcgcccccgagcgctccagcgctgggctacggctaccacttcggcaacggctactacagctgccgtatgtcgcacggcgtgggcttacagcagaatgcgctcaagtcatcgccgcacgcctcgctgggaggctttcccgtggagaagtacatggacgtgtcaggcctggcgagcagcagcgtaccggccaacgaggtgccagcgcgagccaaggaggtatccttctaccagggctatacgagcccttaccagcacgtgcccggctatatcgacatggtgtccactttcggctccggggagcctcggcacgaggcctacatctccatggaggggtaccagtcctggacgctggctaacgggtggaacagccaggtgtactgcaccaaggaccagccacaggggtcccacttttggaaatcttcctttccaggtaggggcgatggagaaaagggaccgacacgagggagggggagagagaaggagaaaagaaaggactaagagtatgcgccc

SEQ ID NO: 476 - DNA, 929 bp, Homo sapiens
gacaagttccggagtgagctcggctgtctgattagaggaaaagagggaagagttacgcgaggcaatcgggagctccgagggtcccgcagcatccttgcctgggtgttcagccctgtcccgacttcgaggcttacctggatgggaaggtgggggccatcagggggtccaggggctggctcggccaggactacctgcaggtcgagaatgtagtcgatgacgcgctgtaggatttccacctggctaagctgagtgcctctcgggactccgggtaccagttcccgcaggcgggagtagcagtggttcatgtcgtccagcaagctcagcggctcctcagctgccgggcccttccctcggccccgggcgatggccagactgcgttccgacaggcagcacaccgcctcgtagcagccgcgcaccgggctcagcgccttcatgctggggagtgagtccagaggtgccccaaagagaaagaaaaccaaaagaagtcccgctacagtgacctgcaacgcgcgcacgctcgccgcggcggtcacttatagagcctgcctggaaggcacgcctctttattcaaaatggcccgcctcggccctgcccccgccggccctgggcgttcacagcccgcttaaattgcaaacaggcttcctccggctggtctgacgccgaagaccgcggagccgcggattcaaagaatgaggaagcgctgataccggggagaggcgggcctctccgccagcaaggatttaaaaatcactcaaaaccattaacttccagaatttgctttttcctggcagcaccccagatctttgagcttccctgccccctgccagtccgcctttagcccaacactggttcgagccacagctcctccgaggtcataaatccctgaacagcaaagaagctccccccaccccccgttttttttaagcgaa

SEQ ID NO: 477 - DNA, 1063 bp, Homo sapiens
aaactttggttcaaatccagatccaccacttgtcagctgtggctctgcggtggacgtattaatctctgagtctcttatttcccagtctgcacaatgggaaaaacgaggacatctgtcccacagaatccttatacgccactacgaaaaagtgacgcacgtcaaaaaaaaccgctgagcatggcgcccggcgaggagagcacgttcgcacacagtgcccgccggacccgctgcgccacggcaaaaaaacaaaaaacaaacaaaaaaaaacacaaagaaacacgcggggttcaacaaggagagggcgaggggtgccacgcgaaccgggcgaggacacggagagcgccaggcagagtagaagggcctctgtctcctcgtgacgccggtcccgcgcggccctctcgctttgtctcaggcacgaacgcgcgcacgaaaccgagaaaccgagaagccgagaagccaaggccgtcaggctctgatgaccggacaaggagcccaaggcgcggggaccgtggcacgcagctcggttggacggcttgggccggcggccgccctctctggacccgggaacccaccggcccagagcgacccgcgataggaacccgggttcctggcctcagcccctctccagagtcggctccaaccccgctcgttttggtactcaccgtgtggagacgccaccgcagctccgtcagtcgcgagtgaagaacctcagaaaccgccgctgtacctcagctgcagcagcaactgcagttccggggcgggacctccacgcacgtactcgtgcgcgctggggaggaagtcccgcccctatggcaaactcagctacctgattggctgcctcgcggaccgcagcagtgccggcgggagagctggcttggggcgctggcacctcctcttacagctttactcctgccagcttgggaaaaggccggagaaggtgaaattctgtgtgctccctccggcgagagactttgtcagctcccgcacagtaacgtaagttttcttgtattcttagtgtagtttcgttaccggaaaggggtctcgatccagaccccaagagagg

SEQ ID NO: 478 - DNA, 1453 bp, Homo sapiens
aatagtgtttataagcattgtgaactttcaacaaagctgaaacgttacgatttcaagatgaaaaaacggctttggggaagggaaatccacctcgagaatgaagacaagtgcggagcgaatagaatttcaggggagatgaaaaggggattgggaaaaacgtgggatctggtaacaaaaaggggggtttgttcttgaagggcgttctgcggggcgggcgttaagaggggtgcgtgcgtgcgcctgggtgaagtttgtactcagggctgcgataatcgaggtgacaaagttgtggaagtagaattcggaggtccgggagcaggaaggggaaggaatgtggggatttccaggctgggctgacaagttccgggcgctgctctcagggggaagggaggtctcctcgagtgccagatagcccgtccgaaaccaccgacactctaactccagctacgcgacccagcacccggcccctcctcacctcaggccgcgccgcctcgaccctccgcgtctccacagcgcacgaaccccttggcgccaggctgggccagacccgtcggcctggcaggcgcggcccccggttcagctgcgccggggcggcccagcgcgactccgcgggccttttggctgctcgccccggctccggaacactgtcagatccttctccgcagaggtagcgcgccaccccagtcccgcggcggggcggggcgctccccaatcccgcccagctgcgcccccggccggccaacgctgcgccccgccccctaccgggacctaggcgctgattggcccctgagagaccctttgcgcccaggcattggtccagtggccgcctgtcgacgagggggcgggactccgaggggtagcgattcccgtaacgcggctcttcgcgtcccggattccggtggctgcgtccgcgtcgcctctcccagactagatcgaagggttccccccacctcccctctgtgggcggaaagagcggaatgttccatttttctgtggaataacggggtcatgaactggagcccgggtgctgcccgccgcccgctgcccccgccctcccttctcgccagggcgcctgcggcctctcttcgcggccttccccgagggactcagagccgcgagggaccgcctagctcccttggggatggggaaggcgtgggagcctcttggggaaagtgaagagctcggtggccgtgactccgcacgagactgcggcagattgtggcggcggcttctctccaagacttacgtaaacgagccttgccacagtacttttttaaattagtaaatgagtttgggtgcgattgatcagtgagccccagaggtggtggcccaccaagcgactagtgtttctttgcggtttgtacctccccgtttatgataaaaaataccgggtaaaaacctattcatcccgcctccgcccccccctttccctcagaaaatggacctcaaggct

SEQ ID NO: 479 - DNA, 975 bp, Homo sapiens
cccgaggaggtaagggccgatgttcccaatctgcagatgtgggtgtactcagtaagtcattcggcctttggtggtaccaggcttacaggagcccacatcccgacccaggtagaggagtggattggagccggggaacactgatgggaccgggcagggacgacaggaggtcagtctcccttcccttctctttaaaatttcccgccagtatggccggctcgcagcacgagttacgccctcttcaggagtccctccccttctgctctcgcccgcctccccgctgccaggcaggctcgaccaatcagaagaggtcttgggcgggcgctgccgtccagcgagccaataggcgggcagtcggagccgggcttgcccgggcatgtgggagctgccggctttccggacgccacgtgcagaccggaagagacacgcggggcttcaggtgagatccaggaccctgcaccggggcggcggggtcgggcgggctgaggccgcgtgtcccgggtcgcaggtccccgcgcgttgtttccgccccggcctgcggcggggacgcctgaactgggctgaccctgccctaccggcccgcagaaaagcaggccgggcgccgcctgtctccagccctcgctctgctcgggggtccagggccagagggggcggcgctggtcgttgggactcggcccagctgccgcggccagcaagtgaccaacgcccgagtccttcccggtgagatcaggcctcccacgcgtcacgcctgggagaacgccagccggcggggaggtcgtgccaggatagcagactttagttgttgtcgttgttccgtggactttttagcagcacaggggaccttatcgagccaggttttttcaggtttgggcgattatcagaattgtggcttgagggtaggggtcgtccgcgctctttttaaaaatctagattctcaggcccctttccccaaaatagcaattaggacggtgtgggagt

SEQ ID NO: 480 - DNA, 1361 bp, Homo sapiens
ctcatccactgagggcgaatgggagccctatgcccaaggcagcaggcagaggccattaggtgggaccttctgaggggcaagtgggtggcacccatccgcaggacctgcgcaccggggcctcccctcaccaagccagggaagagtcctgcagaaagcagccctttctccctagggaagacaacgggcacagggcccactccgggtccccacgcccgcgtgccgggtgacgggaagttcgggttggggcagagggcgcgccgagggggaggaggctgaggaaggagcaggagggcagagaagcaggcgcgccggcggccgaggacgtgacggcagcagcgccggccgccgcgtgacccaagcgggagggagggcggctccgactccggcacccacgggagcagaagggggtgcaggaggccgggcacggcgagctaattgcgggaggtgccctcccctccactttcacttccccaggagagaggaaccggaaccagatgtgcagtgagggacagcagctgggggccatcccccaccccgcctgccgcccgcgctccctcctgcccgtccccgcctgccgcccagcccggtccagcccctacctgcctgtcccgcgcttcccggccggccggcccgctggcgggtggctcggagggcgagctggcgggccggcgggcggcggggctacccggccccagacatggcgcacccgacttgggcgccgctcccgcccgggcagtgagtgaaggaagaagaggcggccgaggaccgcagagcgcggggcgcagtggaaggagatccgggcggtccccgcgtccccgccgcgctgcgccacgcggggactgtgctgccgcgctgctcgctcggccggctcggtcctcccagctcccgcggcgccggggacccagcgttcctccgcgcgccccgctttctctactcccccaacccccgctccgggccgcgggcgccgccgctacccccacccctcctcctcggccggcggccgtgaccctggcaagcgtctgcccaaagcccagccgcctgcagcccgacccctggcggctgggagggccccctgcacccaggtagcttcccaccatccagcccgtgcctcaggacactgaccccaggcgacccagagggccgcggcgcgctcccttcccgcctgggtcctgcagtcagatgtggcaagcatgatgtgggcgcaggtgaagtcccgaaggtctagtctacggggacccaggtggctccccctacttccaggtttagggcacagggcattccctttggaccagaaccagaagctctcagttctcttttttgaaaatgacaagagctttggaaagaggaagagaggccaggac

SEQ ID NO: 481 - DNA, 1413 bp, Homo sapiens
gttatactgtctgcctagtttaggaacttcctttaacagtatttctttactctctgagtttctggtgagctaaaaaaacataacaaaaaacaaaaaactccatcaaaattgtatgcgtgtgctggtcgttgagtacttcggtggagagtgggagacgcatcttcatgaaatttcagaggggaactctgcccatccactccgaaccagccgcagctccacgagcatctcacgccacccctctgcatcaccagggcggccgtggggcgcgcgcgatcagcaggcttcttggcgcccagtccatgcgactatctcttcccagccccgggactaggcccgccccactcggattctccactcttggtagcgcgcgtcccccggactgctgagaccaggcggcgcggaggcaggtgacccgcgtctccagtcccgggcgcagcccagggtacgttgtcagcatcgcggagcgcggccctgggcctctgcagccatcttcagggagggaggcgcggcctcccgacgcggacccgcccccgccgcccgggccgccccgcccggctccgcagagcgccgcgcctaggttgcgacttcccttccctaccctgctcggctgcgtagtgcgctccccgcccagcctgcagagctcgcgccgcggcagcccagccgctcggccccgccgcgctcgcagaggccgccatgggcaccgcgcgctggctcgcgctgggcagcctcttcgccctggctgggctgctggaaggccggctcgtgggcgaggaggaagccggctttggcgaatgtgacaagttcttctacgccgggaccccgcctgcggggctggcggccgattcccacgtgaagatctgtcagcgcgcggagggtgctgagcgcttcgccaccctctacagcacccgggaccgcatccccgtgtactccgcgttccgcgccccgcgccctgcgcccggcggcgccgagcagcgatggctggtggagccgcaggtaagcgaagtggttcccgagccgggctgcgggcgccggagaccgtgccgctggacatgcccccagttgcagctaccgcgaggggcgggccgggaacagcaatccctacgccttcggtgccttgggccaagaaacccgggttccaggtccaccctgttgcgtccccggagttagcctgcctcgcccctgtcgagctacaataagagcctggcgatctgcgagccttcacttcatgagctgcagggcgggaggtcccctgtgggaggaggttctccctgcatttctgagcagacatccgcagtgtttcattcctgaatggtgaagagcttgatacagagaccctggagaccgttcagatccgcagaaagcctaggctgggcgctcacaggccttcgtgcaacttattgccatagcactcat

SEQ ID NO: 482 - DNA, 1604 bp, Homo sapiens
ggtattttaccggacttcccagaatccggatcggggaaggcaccctctgggggctgggggaaccaggaggcccgtggggtaggcagggtccggggaggggcaggtggaggcagctttgtgggcccagctggggctgactctgctgggcttttgccctcaggtaaagccgaactcctaacctcaggtgccaaatcccccacgggggcctccgaccacttcctgggccgccgtggcagccccttgctgagctggtccgcggtggcgcagaccaagcggaaggcggtggcagcggccagcaaggggccgggggtgctgcagaacctcttccagctcaacggcagcagcaagaagctgcgggcccgcgaggccctgttccccgtgcacagcgtggccacacccatatttggcaacggcttccgcgccgactccttcagcagcctggccagctcctacgcgcccttcgtcggggggaccgggccgggcctccccaggggagcccacaagctgctgcgggctaagaaggccgagagggtggaggccgagaagggtgggcggcggcgggcgggcggtgagttcctggtcaagctggaccacgagggtgtgacctcccccaagaacaagacctgcaaggcgttgctcatgggggacaaggacttcagccccaagctcgggcggcccctgcccagccccagctatgtgcacccggcccttgtgggcaaggacaagaaggggcgggcacccatccccccgctgcccatggggctggcgctgcgcaagtacgcgggccaggcagagttcccgctgccctacgacagcgactgccacagctccttctcggacgaggacgaggacgggccggggctggcggccggcgtgccctcccgcttcctcgcccgcctgtccgtgtcctcttcctcctctggctcgtccacctcctcctcctcaggctccgtgtccacctccagcctctgctcctccgacaacgaggactcgtcctacagctcagacgacgaggacccggctctgctgctgcagacctgcctcacccaccccgtgcccaccctcctggcccagcccgaggccctgcgctccaagggcagcggccctcacgcgcatgcccagcgctgcttcctgtccagggccacggtggctggcaccggtgcgggctcaggccccagcagcagcagcaaatccaagctcaagcgcaaagaggccctgagcttctccaaagccaaagagctctcccggaggcagcggccgccctccgtggaaaaccggccaaagatctcagccttcctgcccgcccggcagctctggaagtggtcggggaatcccacacaggtaggtccagcgggaggcgggaggagctcctggttcccaaggaaaccggggcgggctcatgcgcccctgctgcccttccctctcctttttcatcttcctacttgatttcaagttaaaaaatgtggaaaactcaagggaagaacaaagacccatccatgacccagtgaggcagccgcttccgctggcccctcgctggagcccgtggcctgcgtgacagtagaaatggcttcatgtcgggccgggcctgagctgcactccgcatg

SEQ ID NO: 483 - DNA, 1090 bp, Homo sapiens
gcgcggcccctggcctagacctctgggcactctacagccgggtcccatctcctcccactcttcaggcctcgctgacccgcgtcccttcttcgtcccggggctccaaccttgcccccgccacctccagaggccccacccacttatctgccctgcccagttctcctcccctttccaccaccccggcctctgcatccagccccgaggtcctgccccgcacgtctcctgtccccctttactcggccccacctgtggaccttcccacgtcctaacgggctcctgcccgccgccccgcctggaacctcgcccggcccgcggggctgggtttccgcggcaggtgcactgcacttcccgcggccgggcctccgcccaccttgtcccgcaggtcctggatggtcgtgtagtagtggctgtagtcgcgggagggcccaggcccctgcttctggtaccagtcgcggatcttcacctctagctcgccgttggccgcctccagggcgcgcaccttgtccaggtaggaggccaggcggtcgttgaggttctgcatggttagcttctcgttgcccgccagcagcccgtcggacgcggtcaggacgccgccgtagccgccgccgtaggcccccgaggaggacgaggacacaaagcgggcggaggacacggatacgccgcggccgccggagcccccgtgaatgctgggcgcgcgaaaggcgacccccggcccaaaacgcacggagccgccgcccaggcctccgaaggacgacgtggccgacgactggcgatagctgtaggaagtcatggcgaggcggagcacggacggagcaaccctggtctcagaagctgcgattcgcgggaggagcggcgaggccctcacctggcgccttttatgcccgcggccggtggaggggggaagggaggaatggtgtcaggggcggatatctgagccctgaggaatttgcaggctcctgagagcaaatatgggctctctccccattggtcaattccctcccctcccagagaccagaggcccctgccctccagaggtgccccgccccggtccgcgcagaagctccgacccgcactcccccactctctctct

SEQ ID NO: 484 - DNA, 871 bp, Homo sapiens
accccccgcccctcccggctccctccggcgctcacctggacagcgcggaccagtccttcctgctgacagccatgctgcaggagccccgcgtggccgcccgcgccccgccggccgccgcctcacctggcgcccctccccttccagcccaggtgggatcacatggccgcttatctcacccccgcgcgccccggccgcgctccgatttgccgacccgggaatggcctctgggatgtgggcgtcccagacaaagccgcattgattgcagcccaggccggcccggccgcgaggccgcaagcactgggtggaccgtcggacggatcgcgggaccgaacgacggacgcccggggccggcgtggggctggtcggctgcccggggcgagggcgggagacttcctgctcagaaccagtccgaccagcttggcgctgggacgcctccccgcgctgtgccgcggcccggggtgccgaggagctgggctgggggcggggacccctgccgggaacccgacttagcccggagacgctcaagagcaagaagccgagcggaaaagttagcgcaggtactgtgatctcgggtctagagcgcccgcgccttcggggccccttcgcagggagcgccctccccactccctggcttcggaacccacccgggcccaccgtcccagtaggcctcgctgagatgaatgaggcattgctttctgagctgcacacaaaacatgcccctggtagacccagccccacccaaagggagaatcttagactcaattcagcaaacatttgtgaaagcacaaatatgtcaaaggcactgtccttgctccctgatactggactttgggctagagtggcagggtgtataataagggcagaggaagct

SEQ ID NO: 485 - DNA, 1384 bp, Homo sapiens
ttcccccttgccttctcctctctgatccattgccacacacgtgggaaggtgacaacccttccgaataaaaatgaaagctttcttctttagatggaacccccaaattccctcattatttataatgtcaggctgtcctggacaagggaagctgtgcacccgctgacaccagtaagaaggttgccgccatgtcagagatgtccgcggacacctccctgggctccgggtcctcccctgcgctcgcctggagtgggaccttcgcgtgcacactggccttcccacgcgccccgctgcgatggcacccgcgccgggccccctagctcacacagtcggagcgtgctcagcgcgtggccacctcctgccaggtcccagccgggttccaccccctccttttcccctcctcttcttcctccccctccgagttcccctggctctgaccgcgctggcctgggccggagagcccaggaggcgtgtctcagagaaaagatataagcggcccccggacgctaaagcggtgccagcggcggagtctccaactgggagagctgcagctgccgagaggaggagaacgctgaggtcggtcggaccaacggacgcgctgaccgctgccaactgcagctcgcgctgcctcctgctcgcgccgtgccactaaggtagtccgcctttctatgagccctccccaagattagctgggtgcggggtggtgggagccgttctttggtggctgaagcccctctcctgctgctcctcctgcaggtcattcccgcctccgagagcccagagccgagatggaaacggtccaggagctgatccccctggccaaggagatgatggcccagaagcgcaaggggaagatggtgaagctgtacgtgctgggcagcgtgctggccctcttcggcgtggtgctcggcctgatggagactgtgtgcagccccttcacggccgccagacgtctgcgggaccaggaggcagccgtggcggagctgcaggccgccctggagcgacaggctctccagaagcaagccctgcaggagaaaggcaagcagcaggacacggtcctcggcggccgggccctgtccaaccggcagcacgcctcctaggaactgtgggagaccagcggagtgggagggagacgcagtagacagagacagaccgagagaggaatggagagacagagggggcgcgcgcacaggagcctgactccgctgggagagtgcaggagcacgtgctgttttttatttggacttaacttcagagaaaccgctgacatctagaactgacctaccacaagcatccaccaaaggagtttgggattgagttttgctgctgtgcagcactgcattgtcatgacatttccaacactgtgtgaattatctaaatgcgtcta

SEQ ID NO: 486 - DNA, 1893 bp, Homo sapiens
aaacataaataaacaaaccaaaaaaaaaaaaaaaaaaaaaaccacggtagaagaacgattttatgagaactcgaacaattccaggtagtaggcatcaggagtgcccctattttacacaaaagaatacagagggttcggagacttcagaccaagtcactcgcccaaggtcacaagcaggcagaggcaactcctgaccctgcgggttcccaggagtcccgagttcggcgctgatcagcatccccatccccggccgggccaggccttacctgcgcggctgagcgctctgcgtggaggtcctgggcgcgcaggtgccaccgcgccgtgtggtacagccccagggctccccccagggccagcgccgcagctcccagcagccgcgggctccccttacgagctgcagccacggggctcgggccgcccgccgcccccgcgaagccagcccggctctgcgtgggtagcagcggctgggggcggcctcccagcctccaggccaaggcgcacccaccaggccacagcgcccgcaccacccgcgcagccgggtccatgttcgctccgccggcgccgcgggcgggcgcgcgaaacgaagacgccgaggcacgcgcggcgtttaaagggccaggactctggcgccccgcgggttggccggggtgagggcgacgctaagggaaccctcagcgctctcgggactgggcgtgtgcccggcgcccaagttcgaaacgcccgccagagccgcagaggcccgctcgggaacgtttgcagaccgttccatgcttccgcctccaaaggctcttgggtagtgacccggccgactgcaggggggcgaacgtctcctcgccccaccttgaccgcctaaagggcggcgtggccgtctgtaacacgtacttgcccacctctcttacgggcgcccattcaaaacaactccagcccttcctagagtcagtacaggcgccgcgacttccggccactgaaagccccggaaacgacccgacccggcaccaggtgttgcggttctctctgccctcccgcctcattgtccgcttcctactgtgacccgaaccataaaattttcccgaacccgaaggggccttggaaaccacgtgtctctgatttcgcccacgcgaagcggccggcccgccgaccccgagatttccagggagaggatggcaaagtggggtgccaggctgctcggtccttctgctgggctcccccgccctagctgaagcccggagcctccaccgcataggcgtctccgcggacagccccgggatggccccgcccggcgcccgagagggggcgggactcgcagagtgggcggggagaggggcggggcgaggggggagctctgagtcccggctctgctgctccgccgcgtcccactcttctcgctccgctttcgccccgcatcttccaccttccttccagtccttgcggcgaccgagcccgcagtgtcagtccccagcggggacctgagtatgccggtgaagacgagagcggaaggcgaggacgacggcttcggggaagcgggtgacccgaggagactgctggagcgaccttggcgtttccgggggtgccttcccgggaaggggaatcgggatgttggcttcgaagggaccgaagggcccacctcgacccgccccgagtgggtttggagttgtaggtgctgtctcggatgcagggcgagcacccgcgagcgggtaaccagtcccgtgagagctgcaggtccccagccccgttttacagatagggaaactgaggcagctgctgggacactcgcccatatgggttttgctccacccacttccttcagccactttacagaccaggagttgagggactgctcgagcttagagtgtttaggggtggttgagggtgacccccatgttctgtgctcta

SEQ ID NO: 487 - DNA, 1016 bp, Homo sapiens
cccgttttcttgtcgttacaactctggtaacgccctgaaattgtcttgtccattcactgttacgtaatatctgtctctgccactagaaagtaagcgccatgcaatccggatcatgttttcttatttccaccttctgccgtggacctctggcattctcgtctgtaaaacttaggggagatggtgaggaaagggagaaacacgaggtctagccggtccacccgggtctcaagccccgcccaactcaggcctcgcgttccttccaggctgggcaccgcctttggtcacccacgtggcccgcagtaaggccccgccctttgtgggtgtggcctttcacggcgtgcgggccgtccaccggcggcgagccacgtcacacgcacgcaggccatgcgccgcgcctgttctcgccgcgggcggaaaaggggcgtggccatgcgcgaggaccccgtagccgccagcgccaccgcctgagtcgcctgcaggtggctgttcgcgtctccctgccgtgcaggcagctcgaaagccactctgttgtggggtcctcgcttcccttccacgtctcccggaagcggtagcagggccccggcgggtcggggaggaggtttactcagcttgggccccctccgggccagccgccgagggggcgcggcccaggacggcggctaggccgtagtgcagcctctccggagtcctcaggtgaggcggaggcgagggcccactggacgagacgggggtgttaacggtcctaggagcgggggcgcagtcccttcagtggagaccctggtctctccgttttctttcgggtcgcgctcatcttcgccctgttaggggctgaggagggtaactggagagactccttgggcttcagccgacgtcgtttactgccctcctctgtcttcctttctcctgacccctgcctggcctggccgttcttcccgcacggaacgagatgggtggggagcagagtgggggtctctgctcgcttacctgagattccccaaactgacagaccccagggc

SEQ ID NO: 488 - DNA, 997 bp, Homo sapiens
ccgggctggtcatgaggactcagcccttaccagccccactcccgccatcctgcctctccagggccgcagaggccctggggtggacttctggcatttgtcacctgcctggtgcctcataggcatctgggctgtgacgcttaggattcctaaatagtctctcgctttttacacaaaaagcagaaacctgcccagtgtcatccggcaaacccacgacgggcaaggtctccccgcgcaggtcaggtggccgcagggtgagggccagggcaacccggcccacgagcccctcggtggggaaggggcgtgtctccacgcggctccgccccggccaatggggagaggccccgcccctgccgcggcgggcccgccccccgggactcttaaggcgcgacctgcctccagcgagccgactccggagcccgagcccggggcgggtggacgcggactcgaacgcagttgcttcgggacccaggaccccctcgggcccgacccgccaggaaagactgaggccgcggcctgccccgcccggctccctgcgccgccgccgcctcccggtgagtttctgtggggctggggggctgcgggcggggaagggggcgccacggccaggctgtgcacacgcgcggggaccgccgccgcgcgcacacacgcactctcgggcgggaccgcggccccagccccacacggtcacctgggccgcccggcgggcaggccctgtacgctccctcccccgccgtcctggggcgccgacagccccgcggccggccagggtgcgcgcctgagtgtgcggtgagcccgcgaggctgcaagcagacaaaataaacaccccgtcccatgcttcctcctcccacggctgggcctccggcatgaccagttttatgaatcaaacccctttgagggggatagccaagccacaggcttgggtcacttttccctcctggggtccggcagtaggtgctgctgtgcagaagggctgcccggggtcctctggacccttgtc

SEQ ID NO: 489 - DNA, 724 bp, Homo sapiens
gaggggatggggagggactgggaggaaggagagggccaggggaggaagaagaggggctgtggagggccaggggaggagtgggagagaccgggagaggtggaaggacgcgggggaggggagctggagggagggaaagtaccttcccgcgtaaagtccccttctgaaccccgaggtggaaagccctgacctccctgagccccgggccctgggcttacccggtaggtgctctccgtgagccggttcacctcctcgggcccccgggacatggctgcggccgccgggcgagcaagcgcgggaggacgcggctgggcctcgtggccgccggactccgggcagcgggagggccggggcgcgactaaggggctctggagggtcggccgccgccgcagccgtcggcccgagagtgcccgcgcgcgtctccgctgcgaaaatgtcaaaacttgcggcagcgccgccctggccttcttcgaggagcagaggagaagcggccggacgccgccagaggaagctgggcgggccgggcggccccacctgaggcgggcggggcagacgggggcggggccgcagggccggaggggcgtggcgtacccacctgaggcggcctggtgagcggggcccggccggggtgggcggggaggaggcggggccaggcagctggaatgggcgtggccagagggaagcgggccccccgagtgggcggggtcaccccacctgtggcggacag

SEQ ID NO: 490 - DNA, 1406 bp, Homo sapiens
tcccctttttctcttgtcctcttctggtttccctgaatatctcctcaggctccaggtaccaacagctctccacaggagcaggtctgaggccgcatgcagtttgacggggtgttaccttcatacgctctctccaggtcctttcaatgggccaccaaatttacttagatctctcaaggcctgagaaagtcaggcaaaggttcgcaaatcagcagcgaacccagtttcgccaaagcgcggtaacaggtaccctagggttccacagctaggagttctggtgacgccatccaccaggtgtcaggagagggccgggaaggtcaggacccgcgcccaggctccagagcggagtgggcgcagcgcagtagccgggccgggaccgggaggcgacggccgtgggccgccttctggctcgcctcggacgctgggacgccggactggccgccccctccgggcctgctttcgccgtccagggcgcggtccgcgcacagcctgccaagcctcactgggccccagcccgcgccccgcgccccggagatcctcgacgcccctgcgaacgcggggtggacgcgacgggctgggttggtcctcgccccgcggccccgcccccgtccccgcccccgccccgagccccccgcgttaccagcaccccgcggagcgcgctgcgctgggggcggccggtgcgtcggggagcggcgttcccagccgccgcgaccctctgccccagcggagcgctgagcttcggccgctccgggtttcggttcctgccacggccgaggtggctgcggcgaatgtgggcgacccggctctccggcgcccccgcccttccctcgtgctcacctttccagtcggacgggctgctggtgaagggccggcgccgctccgcgcgtccttttgaactcaacgggggcgggcaccgcggagtcgcggaggccagcagaggccgaacgaggaccccgagcggaggaagccgcgggtggcgcgcggggttggcgcagaggccggagggggtggggggcaggccgacggggtgggacaggaaaagcggagagaaaccgccctctgcaggtccccttggctcccccgggaggaaaggcagcctgcccttctccgattgtcactttactctccatccggagccgcttcctttctcgccgcgaggctcggggttggggggggaccagattggagccgcgggctaactgggatccgtcccatttccctgggcttgacgttctctgaatttttagctaatgtggaaagttacatttatttgcatttgtttatcgcttgctcacataggtctgtgtcctgaagcttggcagatgagcgaacttagccagcacacccccggccgtgaagcagggaggtgaagcggggagagcaacgagccccacccgggtcttgccagc

SEQ ID NO: 491 - DNA, 1262 bp, Homo sapiens
cctccccacgaccaggcctctccccgctcctctccccttctcccttcctccgtccctcccctccccctagacacatacaacaaggccacctccccccgaaccccaccaactttccagtgcccccactcttcccacctctcaggagcacacagaagctgcaggccaggaggctcctcaggacggcccccatcttcccttctcgcgttctcttctctcaccggcgaggggtggccagaagtgggggaggcgaggagccggggcggccgccgcagcggcagggctgcacgctcatacttcccagcttcccagcgagagaagaaagaaaggggcacgtcaaggttccgcgcgcgccgccgccgccctctctaccgcgccgctcggccccgggctcgcgcgacgccagcgtctgcttccatcccgccgcctcctcccccggcgccccgcttcccccgcgcagctcctcattcagcctctgcctcgcgcagcggcgcagggatacggtcggcaccgcctcctagggtcgccgcgaccgtgtccctgcgcgcaacgagccccttctcccggtactgcctcctgtacggccagggaagggagtcggcaaccgcacgcacagcctctgcctgagaccctgggagaggttctgggctagcagaggcgaactgggagggaaagcctcctcccggtggcgcattccagcgggattctttcccggaccggccgcttcggccctccccgcggagggcgtgaggcggcgttgaacaacattggagccggcgtggtcgggactactttctgcggctcggccgggcagccgtctgccccgctctttgtgcggccgccgccggcagggccaggtggggctccggtctcgcgcccccagccacctgagactgcccagccgcggtgcacgcgcggggagccacggcggatcccgttgcggggtgatgagctccgtcttcggggttggaaatcggtttcagcatcctttttttgggagggcggaatttttgaacgctgtttacaccggctttcagcgtggatctggtgaacgatattaaaacgccaattcaaaactgagattgtcatttttctcccgccttcccagaaatacaaactgcccccagaattaagcccgaccagctaaagatttctaaactggcagataaaattccaagataaatgcctcaaaacctaagagcaaagggacaattggacaagactatttaggattttacaagttgcatataataacgaatgtttgg

SEQ ID NO: 492 - DNA, 427 bp, Homo sapiens
gggtcctgaccttgattcctgccacagcgtgggccctgacctcttctaaccagccctgccctggccacattaagctctgtccccagagttccccgtggctaaagcggcgggggtcggcggggtgaggtagtggcgacccagcctggaagtcttcccctgcggggtggcccaggcccggcagcagaggcagcactggattatttgaggccctctgtctgaggtgaggcccgcctcagtcctccctcagcgtcttaccttgcctcctcaccgagcctgggccggcttccctccgccgacgtcaggccgtcgctcgttgctcagggcgagaatctcgcggtcttctgacctccaatgcgcaagtcagtggcgtcacatcctggcaacggtactaccctgggttccgggcaggggtggaactggattctgc

SEQ ID NO: 493 - DNA, 1718 bp, Homo sapiens
gggaagccttgctgctggtcttgggaagccttgctgctggtcttggggacgtgatcccctgtccctaagtgtgcccctggaggttgtgggggaggggtatcctcaacacagaggcctgaccaccaaatcccgggctgcagagccaacaatcctcctttgggctcctgcacagactaaaaacaaaaaactctgagaactccgacaagtcaacgaaagtctgatcccgtcaagcccgcactcgcaaacccccaccgtgctgcactgggccccggccctcccgagctcggtcccctcggacgacggtcgtcgccactgcgggcaccagattggggggcgggccgctcaccccgcggtcctgccccacaccgaccccgctgaggagcccccggcacccggggcgaccagggaaggcgggaggaggtgcccagaaactgctcgtcctgccagtcgcacctggccgacgactctctggccactggtttggtctgggggatacgatttcccattcaactgaattagacccgaggccgcggcggggagggcagcggccggggtctctccgtttcctcccccggctcgcctccacctgttgcgggaaagtcgcggtggagcgcccgagggcggccgcaaccccggccggggccgagccgggctggccgagccgcggcggcgaagggcgggggaaggcgctctcggagcagggtgcgcggcgcgcagggccggttgttccgggccgcggccgcgcgggcggggagggggctgccggcgccgggtgatttcggtgccagcccgccggccccgctcgccgtcccctgcccgacgggacccccactatccccgctgtgcggtcacctgtttcgcttcgcactgcgccgtcgccgcccctgtggccgtcgccgccagctcctagctccgccatggtgctccgatgacgcgcccggagagccgctccgccctttcctcggcttggctcccgccggcccccgcctcccaggccagcggcccagacaggtgagcgcagtccggggatggcggagggcggggacagagagcgcggcagtcggctgcggcctgggcctggcgcgccccctcgcgggcaggtgcggcgtggcagccgctccgaccgccgctgccgagtctcggctggaagagcggcgggcggaaccagtacctcagtccggagcttccggtcgccgcggccgaccagctgagggctcccggagatatggagcggggtcagcgaggcctggtggcgcctcctggagtctgggacgagcggcggtccgaggagcagcccaggtggggcgagggtacggagacgctatttccctccgaactctgaagtagccaggtcagtgtcggctccgcgagggctggagttcacggtgacagccggcagagcatctgatgccacgtggagaggcgaagtacatgggtcggattccacgctgggagccggcctggatctgcgctttaggaagccactgcgggcgaccctatcttgtcgccaggacttcgcatctgatgagcctcagggcacacaagttagagaggcttgtgccggctgatgtggcttttctttttcttagtttccgtttagtaaacgaccttgtgacttctggcttgctcaggaagaccagaaaaaaaggcaataggtaacgaatttttctttttttaaagaaacagattattggccgaggaaaaaccaatttcgctct

SEQ ID NO: 494 - DNA, 2296 bp, Homo sapiens
ttgtaggtacaggcagcttttgtctgaaatctcagcttccgaagcggatttctttttctctggcactgggaggtttcgccgagccgggttggggacgggaaagaggagcgcggggaaggatgtctggggtggtgagggcagggcttcgctggagaaagagctagtggggcgcgaggttcttacaggcccgggagaggtcgaggctggagcccctcggcgcctctaagacaaaggcagcggtggctgcagccggagcctagcactccggcagcgtcgcgccgcgccgcgccgcgccctgggcgcacggccgcctcaccccgagcgggtcggagaaagagcctccctcccagcggctccccggccccggctccgcccgcgaggtctgggctgctgcgagcccgcgccgggtttcgctttccgacgatcataaatagcttggtgtttgtaaacaggcgctgggggcacattccccgcgctcagctcattgttccctcccttcctctctacttcgcgcaggacgcctgggctggggctgggagccgccggggctaggaggtgggggagtccagacccgaagtgacaaaatgctagcattttctttcccccggccgggcgtccacctctaacagcaaagaaacctctaagctgggtctgcagaaagcccgagccacctcaaccccatgttctcaggactccttagcagaggctttcccaacctggcttctccctccttttcctccacgatcccgctttgacttttctccttgcagtgtttagttctgagaattcagtactttgcgcatcaccccctgccccgaaaaacactggcagaccccaataatttcgaggaaagtcatgaagtctatgcgcggagccctgtgcaaaataactcccgctgctgcctgcccggcgttgattcccaatttatttcaagagagtcggctttggggagagagtctgcagggggagggagagaaaagaatactgaaaataaagctggcggccgcgggctactgcctgcgtttgtgtgcgtgtgccctgggtgtgtggtgtttgtgcctgggcgtgcgatttaatggagcgcctctctgcctctccagtgcggccagagctcgcttcgcgcacccacccctgccgaggagcctacttgctgcagcccaatgcattgtgtaagacgcgacctgttatggccaccactacttccgggttctagcattctggtcggaatccacctctccgcctgtgcaacacacactttacacacgcacggggactgcaagcgggcagcatcgatcgtggctcctttaagacaaactcagacagacattttttttaaccctccttctctaatctccttccagtgcagcacttgcaaagagggagagagagggagagaaagaaagagagagagagaagagagaaactgattaggaattaggactgattcaagggaagcgagcgctagggctttgtgcatttgaatattaacatttgaggtgttctgaccagaagaagacagagcggatgatcattcattcaccacgttgacaacctcgcctgtgattgacagctggagtggcagaaagccatgagatttggtagttgggtctgaggggcgctcttttttttccttttctttctttctttctttttttttttttaaactgatttttgggggagagaagatctgcttttttttgcccccgctgctgtcttggaaacggagcgcttttatgctcagtgactcgggcgctttgcttcaggtcccgtagaccgaagatctgggaccagtagctcacgttgctggagacgttaagggatttttcgtcgtgcttttttttttttttttttttttttccgggggagtttgaatatttgtttcttttcacactggccttaaagaggatatattagaagttgaagtaggaagggagccagagaggccgatggcgcaaagggtacgtattaaaaaacaattgtggaactcaaatgtgagattaaattgaaacgtggccgaaagacactgctaaaaagtttcctttttttctctctctcttttaaaaaatgaggctcctaaagccgtggcctaagcgagaggatttattcaatgcatttgtagacttatgtatgttacttggagttgttaattaaaggaatctatttatctatagattcctaaccagcagctatataaataaaaatgcaagctcaaacaatcatctgggtttttttttaaaaaagctagatacctaaagctgtaatttgaacattcagagatcagaagtttaaagttatg

SEQ ID NO: 495 - DNA, 1257 bp, Homo sapiens
cccttccctgccagggcgctgaactggccctgacccagtgaacgccggcagaggcaaagcacctcctcgccgcccgctcggggggtccagaccctccccaggctagatggccggcccctgtatcctcagaccgggggccgccgggtgagccgcccccggccctccggcccctcggctctccccccgccccccgccactccgccagcccttccgccacgactcccgtcccagactctttcctcgtagctcctgacacactaacccggccagtccaggcccctctccccgaggcgcccctgcccccgccgccccttctcctcccggaagggctgggtgtggccgcccggaccccactttcaccctccatcccgttaccgcggctccccgctgcgtgaccgggtcgccagctcacacccacctggtgctgggtgtgggggttgctgcacgagggggagcaaatcactgcgtccgctccccacggccggcggcgctgtctcttctcggcacccagcacacaccggctccaggctccgatcagctggcggcgtcgtgacgcacgagctgcgaatgcacggcccggggccaatcaccgcccactgctgggcaacgtggccccgcccacccgggggccgacggggcctcgccctggtcccgcctctccgggcgccctggctctgattggcaaggaggccacccttttgccgggcggaattgcacgaagccacgcccacccactgaggagccggccaatagacggacaggtgtggcccagccctccgcgtcagcgacccgcttagaaaagcgctacgtcaaacgagagtcctaaagagcaggcgaaagccacggcgtctgcgtttgcaatgcatgctggtccgtgtagttctcagcctgacaccgtcgttcccagaacccagcgcgctctgtgagttggcatttttaaactgagctcggaatccagcccggggaagtccttaaagggcgacagcccctccttgttggagtcgtgagtgcttcttttagacattccccaaactgactcgccggcctctgcgggacgctatagctctttaaggaaaggtggagcggaccaaggagcaggagtgggcagagcgtcagcccccagggcagcagagactgccgttggagagctctccagggtttttggaggagggagggtgctcgcctcgcccagcccactcctcctgggggctcttcctaaccacaggaggctttttttcttggatgtctcttcccagt

SEQ ID NO: 496 - DNA, 841 bp, Homo sapiens
tcctctccccttactccttttccagtcctccccaccccccagcatttcgtgggtggggactgaagataggttttcttcgttgtcctgtgggtggtattctgattgggaagggatttttttcaattgtttacattctacctgagcagggaaagggggtgccaaaatgaagcatcattcacgtccagtcacatatgcagagacggggcaaacgtttgatgcaatcttgggtctcgctgctcccggggccgacaccgcaggcgatggcctaagacttacctgctgggtaggcccgaacggcagtcccagacttaccctggcagcggaaacaataccccagagcaccgatcgctcactgcagaactgttcccgctgctagggcacggctgagcgcgggcaccgcgtctctcctcgactcgcaccgcctatgcgccccagcctagccaggaatactgccctccccggactacagccctgctgcgtggacaagggcgaggacccgccggaaacagagacgcgagcccccgcccacctacctgctcctgagccggcctcgcggggagcgggcacctggtggcaggaacacgcgtccggcagaccccaccttctaccttccccacgagggggtatagcagcgcctcacgttttgaaccatttagaacacttcaaaacatgaattgctgctgtatcccctcggagtcaatgaagggggatcgatctgggggtacctgcgggtgaataaagactggatgactgaaggaagccccgccccaccccctggcaaggaatagggcaaaacttatgactaggccacttcactccaagttaaggtacttcttataac

SEQ ID NO: 497 - DNA, 961 bp, Homo sapiens
caccacaggccctggaggctgcccccacggccccctgacagggtctctgctggtctgggggtccctgactaggggagcggcaccaggaggggagagactcgcgctccgggctcagcgtagccgccccgagcaggaccgggattctcactaagcgggcgccgtcctacgacccccgcgcgctttcaggaccactcgggcacgtggcaggtcgcttgcacgcccgcggactatccctgtgacaggaaaaggtacgggccatttggcaaactaaggcacagagcctcaggcggaagctgggaaggcgccgcccggcttgtaccggccgaagggccatccgggtcaggcgcacagggcagcggcgctgccggaggaccagggccggcgtgccggcgtccagcgaggatgcgcagactgcctcaggcccggcgccgccgcacagggcatgcgccgacccggtcgggcgggaacaccccgcccctcccgggctccgccccagctccgcccccgcgcgccccggccccgcccccgcgcgctctcttgcttttctcaggtcctcggctccgccccgctctagaccccgccccacgccgccatccccgtgcccctcggccccgcccccgcgccccggatatgctgggacagcccgcgcccctagaacgctttgcgtcccgacgcccgcaggtcctcgcggtgcgcaccgtttgcgacttggtgagtgtctgggtcgcctcgctcccggaagagtgcggagctctccctcgggacggtggcagcctcgagtggtcctgcaggcgccctcacttcgccgtcgggtgtggggccgccctgacccccacccatcccgggcgagctccaggtgcgccccaagtgcctcccaggtgttgcccagcctttccccgggcctggggttcctggactaggctgcgctgcagtgactgtggactggcgtgtggcgggggtc

SEQ ID NO: 498 - DNA, 357 bp, Homo sapiens
gggcctgtcttcagaagagaaaatgggggaagagctttgaacccagaggtacaatctctggattggagagtttcaaaggtgccagggaaaagcagtacaaagagccttctttttgacaataactctaaaacacactttgctctgtgaacgcccgtcctgggcagggcctatcagcagggccagatgagcctatagacacccagggtggcgcgttcccagccgccctgcgcatgccaatcttatcagttttgatggaagcctggggctccagataatgctacttggccccaaacgctggacagagcatgctttttcctgtttggctgggaaagggaaggctgaactgctgagtctgac

SEQ ID NO: 499 - DNA, 1630 bp, Homo sapiens
tcgaatcattacatatgaaaattcagagaatagcaaaacaaaataagacgcaaaggggaacaggtctatctgcttaaatactgaaagattttacatctccaaaaagcatttgcgagatgccaagatttctcaaatgggcccctcccccgtcgtgagcctctttcctccccctaccaattctgggaagtcattagttggacgcggctgggatgctactcaccgagtttctccaccagcttaagcatcacgcctccaatgtgcacctcgccggtcactctcagggtgacatcgcggttcaggtccgtcacatggacactcagttcccacgtcccgtccgcgtagcagccatctggcatccttatcccgtccagagccatggctccttcctgcgagcgcggaggaaatggctctcgtaagcgtcactcccccaaagaaaggcgaatcccaccgaattcgcagcgccggccacgggctgggaggtggtggaggacccggggctttcggcagaaactcgggagggcggcggcggccggggctgcgggaggaccctacgccccgctcgccccgcagcgttcctcggtcccggcgccgcccgccccacaccgtccccgcctagcggaccgcggagggcttcacctgccggccggccccgagacacagcgcgggctgactccccgttcccctccccgccgcgccccctcgggtcccggcggggtcccgctccctcaccgcgcgctcctagcgctccgggcccgggactcgcgcggcaacaggcgaggggctggaggctcgcggggcggcggtgtccgcgtccggttcctcggctggcgaagcgctgcctgtggccggagcggctaatggagtcccgtcgtcgcggcccctcgcctggctccccgcgctccaccctctccccgccccctctgtccgagcctggctgggctgggcgcgctggctccctccttcgccgcccgcagagctgccctgagcgggtccggccggcctcgcctctgcccccgccctcccggcgccgcgctcgggggagggggctgcgggtcccgggcggggcggggcggcgtggggcggggcggcgcgggggtggcggggggcgcggcggggcggcaatctcggccccgcggagcgctgcccttttatggaaatgaaggacccggctcaggaattgaatccaacatccgggctctaggcggcgggccctgaggagggggagcgggggagcgcgggcggcctttcccggggcgctggtggccggcggggcgtctagagctcaggctggactggggcccccgcggcgtgtgtccgcgctggcagcagggagagggggcttgtccgtgagaaacggccccaggaaattcccagggcaaggtcgttttcggagaacaaagtcctctcgccccctccccacgcctgcagcgagacaaaaagcctcagggaggtgagaggtgccgcaccccgggagctggagagaggagtctgggacggagaaactttaaaaagtaaatgcccaaaccaactccttgaatgtcctacaaaagaataagaaaaccccgagggaggtgaaccacaaaaattgctttgcaagcaatgtccggcaccatcagcaactcctactgga

SEQ ID NO: 500 - DNA, 738 bp, Homo sapiens
aggaggctgaggtaggagaattgcctggacccgggaggtcgaggctgtagtgagccgttattgcaccactgcactccggcctgggcaacagagcaagaccccatctcaaataacagtacaaaataagtatggagatgctggctcctgtgggaagctgggtggacgtggggggcacaccctccagtctgcagaaacttcccgccgctcgggctgatgtgctcgagggctcccgaggccctgcctcccgggtggcagagggaacatgggaaaggctgccgggccccgggctcgccggtgggtggccccgtgcagcctgttattcataaaacttggagagaaagtgcctttttgtaggaaagtgtggggtgctggcggccatttccacctgtggttggacccagactgcctccccgcgtgtggcccatgccccaccccacacgcacccccgccctctgtcaagcaggactcgggctctccgttggccgagcagacgttttatttgtggaacggaattgctgtcgggatgggcgtggggtcgttagcagcataatcaacacccacatccaccctgggggcagccacgtcttgtcaacaggtggaggccgtgacatgctgcaattagcagctaatgaggaggcctgtgtgacccacgcctccgtcacaggtgcgcagtgagctgtggaaacgccgacaatttaaaatagtcgaacagggccgggcgtggtgtctcaggcctgt

SEQ ID NO: 501 - DNA, 3190 bp, Homo sapiens
tctccaaatctctcacgacctgatttctacagccgctctacccatgggtcccccacaaatcaggggacagaggagtattgaaagtcagctcagaggtgagcgcgcgcagccagcgtttcccgcggatacagcagtcgggtgttggagaggtttggaaagggcgtgccggagagccaagtgcagccgcctagggctgccggtcgctccctccctccctgcccggtaggggacctagcgcgcacgccagtgtggaggggcgggctggctggccagtctgcgggcccctgcggccaccccggggacccccccaagccccgccccgcagtgttcctattggcctcggactccccctcccccagctgcccgcctgggctccggggcgtttaggctactacggataaatagcccagggcgcctggcgagaagctaggggtgaggaagccctggggcgctgccgccgctttccttaaccacaaatcaggccggacaggagagggaggggtgggggacagtgggtgggcattcagactgccagcactttgctatctacagccggggctcccgagcggcagaaagttccggccactctctgccgcttgggttgggcgaagccaggaccgtgccgcgccaccgccaggatatggagctactgtcgccaccgctccgcgacgtagacctgacggcccccgacggctctctctgctcctttgccacaacggacgacttctatgacgacccgtgtttcgactccccggacctgcgcttcttcgaagacctggacccgcgcctgatgcacgtgggcgcgctcctgaaacccgaagagcactcgcacttccccgcggcggtgcacccggccccgggcgcacgtgaggacgagcatgtgcgcgcgcccagcgggcaccaccaggcgggccgctgcctactgtgggcctgcaaggcgtgcaagcgcaagaccaccaacgccgaccgccgcaaggccgccaccatgcgcgagcggcgccgcctgagcaaagtaaatgaggcctttgagacactcaagcgctgcacgtcgagcaatccaaaccagcggttgcccaaggtggagatcctgcgcaacgccatccgctatatcgagggcctgcaggctctgctgcgcgaccaggacgccgcgccccctggcgccgcagccgccttctatgcgccgggcccgctgcccccgggccgcggcggcgagcactacagcggcgactccgacgcgtccagcccgcgctccaactgctccgacggcatggtaaggccgggaccccaggaagtgaggaagttagggcggcgctcgggatatcagggacgcgtttccgagggcggggagctggccttgcgggaggtttgggccaggatccttcccgagagagaggacccccttgtcctgggcagctgtcactggggtagcctgttttggaagtgtgcgggcaagcgttcgagctgccccattgggggcgctattagaacactgcagcgcgaacgtgaagatctttttctctacttatccctacttccaaaatgtaaatttgcgccccttggtgactgtccgcccttggtttggccctgcatgttgcagacctcatctcctacccacccgtaattacccccccaaccaggacaggtctgggcccggaactagagccttaggctagagttagggagggggcggctacaggaattggtgttcgggcctcgagccgtcccgcgggcctgactcagtcgcccttgctgtttgcagatggactacagcggccccccgagcggcgcccggcggcggaactgctacgaaggcgcctactacaacgaggcgcccagcggtgggtattccgggcctctccctgctcgctcctcctccttcatggagctgtcctggcctctatctaggacgctcccacccccactcacacacgcctatgtcctgggaagtggtgcaggagatgaaatactaagcaagtagctccctgtcttttggattgtcccggactctaactaaagtcctcagtttccaatctgtctcaaagtactgggcccgggggtgggaggcttgtcgcggccccacccctgcttactaaccgagccctccccgcgcagaacccaggcccgggaagagtgcggcggtgtcgagcctagactgcctgtccagcatcgtggagcgcatctccaccgagagccctgcggcgcccgccctcctgctggcggacgtgccttctgagtcgcctccgcgcaggcaagaggctgccgcccccagcgagggagagagcagcggcgaccccacccagtcaccggacgccgccccgcagtgccctgcgggtgcgaaccccaacccgatataccaggtgctctgaggggatggtggccgcccacccgcccgagggatggtgcccctagggtccctcgcgcccaaaagattgaacttaaatgcccccctcccaacagcgctttaaaagcgacctctcttgaggtaggagaggcgggagaactgaagtttccgcccccgccccacagggcaaggacacagcgcggttttttccacgcagcacccttctcggagacccattgcgatggccgctccgtgttcctcggtgggccagagctgaaccttgaggggctaggttcagctttctcgcgccctcccccatgggggtgagaccctcgcagacctaagccctgccccgggatgcaccggttatttgggggggcgtgagacccagtgcactccggtcccaaatgtagcaggtgtaaccgtaacccacccccaacccgtttcccggttcaggaccactttttgtaatacttttgtaatctattcctgtaaataagagttgctttgccagagcaggagcccctggggctgtatttatctctgaggcatggtgtgtggtgctacagggaatttgtacgtttataccgcaggcgggcgagccgcgggcgctcgctcaggtgatcaaaataaaggcgctaatttataccgccgtggctccggctttccctggacatgggtgtgggatccggaggaaaatccgcaaactgggccagctgtccctcagcgacgcctgtaggcggcaggcggattgcaaggaggaagcctgctgcctggggaaggaaggaggggtgcaaatttctccagtacgtgaggaagttcctctgaccttgactacattactacaca

SEQ ID NO: 502 - DNA, 510 bp, Homo sapiens
cctcagattgaggtcccagggccaaaggaccactcctctcctcagcgctggtccgggaaaggcaagctccgggcgggagcgcacgccgcgcccccgaagcctggctccctcgccacgcccacttcctgcccccatcccgcgcctttccaggtcttctcccggtgaaccggatgctctgtcagtctcctcctctgcgtcctcggccgcggcccgggtccctcgcaaagccgctgccatcccggagggcccagccagcgggctcccggaggctggccgggcaggcgtggtgcgcggtaggagctgggcgcgcacggctaccgcgcgtggaggagacactgccctgccgcgatgggggcccggggcgctccttcacgccgtaggcaagcggggcggcggctgcggtacctgcccaccgggagctttcccttccttctcctgctgctgctgctctgcatccagctcgggggaggacagaagaaaaaggaggtagaatggatccccttggccttc

SEQ ID NO: 503 - DNA, 2067 bp, Homo sapiens
gaacccccgaggtgaggctggaagctggggccaggagctccactcttcccaggagtcagagaagtgagttcatttgctggctttttagttcctaataagtccctggggcttttcgggatgcttaagccaagggcgtggggccgagagcgacactaggataacactaggcaaggctgagcaacccaggctaccaacagcacggacttgaaccgggtcgcatgccggagcggacgggatgaggtgcaccgcagaactggggactcctggtgccgggacgcggcgggctggggcgccccactctcggggcaagtctgcaatgcccggtgcccacaaaaaggacacatccatctttgtcaccccgaggcctggcacggcctgacggcccaggctcggtaaaagtcgggcggagagagtggaagcgcagagaaacccggggagggcgcttaagaacgggccagggcgcccggctgggctgccctgcctctcgggcgccggccaggcgtcccggccgccccaccacgccgtcggagcgcgccgaagctcagcccggggcgggcgggccaggagagggcccggggcccctgccgccgcggcgccccctcctgcgcacccggagggagaggccgcgcgcgcccgcccgctctttctgcgcgcggccctccccgctgcccccgccctcgcgccctgtttggagggagccaccctgacgtcggagccgctcccccgcgccgcgcgcccgcccggggccgcagacagcgcgcagcgcagcccagccgagcgtcgcggggccgccccccgccctgccggccgcctcgccgagcctcctggggcgcccgggcccgcgacccccgcacccagctccgcaggaccggcgggcgcgcgcggtaagtcccgcccgggtcggcacgcgggcgcccggcttccagaggcttcggccgcgggggcagtgccgcgcccccagcccggagctgcgtcccccggcgcgtccggcggccgcgggcaccgggtggaggcgccgccgccgagctcaggagccctgggggacctggcgggcgcctccggacgccggcgctggggacgtgggcgcggcacggggtggccggggggcaccgccgcgtccggagcccgtccccagactcgccccagggttccgtttctggatcggagtttccgcggccgggggagagagggagcgagggagggagatgcgtggccgggcgggccgggctcgcatgtcccccggccgtgcccgggcctcgccgcgccgcgcctctcggctctggacgtttccgcctcccgcggccccctctgcttcggtccccacgtccgtgtgtctgtctctccgtcgctccgtttccgccgctcactttcccctggtctgcccgtctctttccgttccccgcctgcctccgtgttcggctgcccgcgtttgtgtctctgcctctggcttttctctccctctctctgtctctgcgctgccctctgtgggggtctcgccctgtgtctgcgcctctccctgatcccccgccctccgatccagacaatcccccagtgtcggaggctcgccttcccccagcggggttcacgaatagtttcctagaccgggtgcggcaaaccctccccctgcctcccgccggccccgaggcctggcccgggtcatctccctggaattgagtctggggcagggagagccagcctttccgggggctcagttttcttaagaattccacccccgcgaggccggctggacgacaggccctgctgcgggccacctgcccggcccgctcccagggcggggcgattctgatcgtttacaagcggcggcaaagggggcagccgcctgccttggcgtggccggaggttggcgggcatcaagggagaaagccacacttccaggctttcggaggccctcgctccccaagctggacgccaccagcctgtaactaggggcaccccaggaaaacagaggtcagggctggaggccaaagaccgggcgctggcggggaagcaatttgtcaacaaggagcccctcaccctttgttactgcggaatggggcctccctagac

SEQ ID NO: 504 - DNA, 2798 bp, Homo sapiens
ctacgcccaaatatcgtcaaatgaacaggtttccacaaaaggaacacgttctaaaaactccttaacaatcaccaagaccaactaactcgtgtcaacgtaaatactgcctttgtggtgcggtcactagaaaccccaaagtgcagcaggtcactatggaccagggtttcagatccccaaataaagcaaaggaatgggtccccggaccacaacctccgactcgattcgtcgcccgcccaccgcctgcagcgcagtccagcggcgcccacacagactcgtgctacagtcccctcgcccaagttcactggctgtttttaaaagcttagtcccccgtaaagtcctccaaagcccgagtccacctcaacgcgcgagctgctgagaggggctacgggcaccccgggcggggatgccgcaccacaacgggcacttgaggggccgcacggggcctcgcacttacaggtcccggccctgcccaccccccggcggccttgcccggcccagcgcccgcgctccgtgcccagggcgcggggctgggcgctcgcagtggccgtggcgaggggccgctcccggctcccacgccgccccgcgacgcccgctgcaactccgggccacttctccaagggacggggatgtgcggaggttaactcaagccgctaattgtccgcaacaaaacaaaacaaaggcatttcgggccagagagaaagctcgagaaagcgaccgagacatgtacctgagtgagacagatgggagaatacatcatgacttcgccttaaaacgcactttccgggagatgcccaagaaaatcttcgagaagcaagaatttcatctattcattttacagtcatctgagccccgcgatgcgatcaatcaggacggggctctgcgctggatcaccgcaacttcacaacaaacccagtcctccttaaatagccaaagattcaagttcactcggcgtgctagatttccaggggtgaaatccaatctacacttttaaccctcttgcagtccgagcgcgctggccgtgcttgccgaggccgccgccgccgccggtgttggctgcttttcgcctgggtttggggatttgttttctattttgcagttgttgttgttgttggggtgtagggggtgcgcgaaggttcggtgtgggttggattggggtggtggttggtggtaaaatgctttttcaaaaaaggcggggaggggggcgcgggagggcgcaggagggcgagcgggcgggcgggagggagagcggggagaatgtgtcaccgcgctgggaaagttcaaggttacagccccaagcactgcggcaggatcccggagtggtgatcgcaggcgaaactttgccgcgagccgaccatgtgtgtgcgcgaggggcagcgtgagcgagtgcgcgcgggtggcggggcgcgcgcgggagagggccggggtgggggcggggtgggatgggggagaagggagaggccggggtgagggagggggcgcgagcgctgatctctggggcggaagaggctcgcggcgcgtggtccccggcagcagcagccgccgccgcccgcgccgggtcttcggcgcccggccgcccgcccgcccgcctcgccccgcgtctgacccggccgagcgtggaggctgctgttgccgccgctgcccagagcccaggggtatcaaaacgatctcctgaaaaattccaaacgataaatcccgctctcccgctcggctccaaactcctctcttttctgctctaggctcttttttttccctccctctcttgctttctttcgttgcaaaacttggaagttgaaaaattcaccggttccaccctctggaccccgctcgagctctctccaaatcagacttaaggattgttttattatttaattacacaatcatcgctttactcctcctcctgcaaacgcgcattccttttgcaccagcctctccacaccccccgcccccccaccttcctcctcctcctccttctccttctctccccccacctctccctcatttgttaaaggtatcctaccggtgctacagaaccatgactttttttttttttccaaaatagctcttctttccgctgccctccccacaccagcacacacatacacacccccgaaaacccgccctggaagaggtatttcggtgctcgctgcggactcttttaagccgcgggaacatccgagtgggagaagcaccgaaaccgtctgagcccgagaaaggaacgagtggaaaattccctcacacccaggccgcccccaggccggggctggggcgcggacactcgcacccggggcgggctcagtccgcggagcctgcggctgccgggaaaagggggaggccgagcaggcggggacctgagcgaacttgggctcaagtagttggggcgcgccggcgtggaccaggggggtgcggcgcccagcccccgacggccccgcccggagcccgggttcgcggatagggagggggtgcgggttcgtcccgggtctgcccaccaggggcccgcacccgaggcagtcgcggcgcgcggcgcttcgccgcggtttgccgccctccccgggggtgccccgtgcacgtggcctcgctctgagcgggaggaccgggccgagccgccgccgccgcctcttgctccctccacctcctccccgcgcccggcttcgctctcctcctcctcctcctgcagccccgctggctggctcccaatccccaccccccggggcccggggcgccggcggaaagcgtctccctacccgcccgggtgcaggcggggcggggccgcggcgcgcccggggctgcggggcgggccg

SEQ ID NO: 505 - DNA, 1537 bp, Homo sapiens
aaaaaccagtagagggttatactttactgggcacaagtcgtttatgataacgaaattgtagtttaatctgtgaagagatgtgaatgtaactgagacacgcttaaatggaatatacagatgagctttatttttatatctggcatgcttggatccatgccgaccctccagctgctcgggcctgcccttaggggctatggaccgcatgactctatcagcggcactgccaccgccgccgcctccgtgctgcctgcgttccccgaccattgattgggcccggcaggcgcttcctgggggcttccctaccggctccagcccttgggattcgggagcgccctgctaggaagccagagccccgcaggggccgcggcgtccaggccgcctaacgcgcgcccctcgcccggcgccccgaagcggccccgaggggcgggagccgaggcgagcggcaaggccgggccgggggcgcacagcgcccctagaagtgcgggcttcccccacccccggcagcgaccctacctcccgcccccgctgcgtgcgcgcgtgtgtccgtctgtctgtatgctctctcgacgtcagtgggaatttccagccaggaagtgagagagtgagcgagacagaaagagagagaagtgcaccagcgagccggggcaggaagaggaggtttcgccaccggagcggcccggcgacgcgctgacagcttcccctgcccttcccgtcggtcgggccgccagccgccgcagccctcggcctgcacgcagccaccggccccgctcccggagcccagcgccgccgaggccgcagccgcccggccagtaaggcggcgccgccgcccggccaccgcgcgccctgcgcttccctccgcccgcgctgcggccatggcgcggcgctgactggcctggcccggccccgccgcgctcccgctcgccccgacccgcactcgggcccgcccgggctccggcctgccgccgcctcttccttctccagccggcaggcccgcgccgcttaggagggagagcccacccgcgccaggaggccgaacgcggactcgccacccgggtaagccaaactcgggcgagtgggggcccggcaggggacgcgtggcccaagtcctgccgcccagccctccgcaccccctccagccccactcggacttcctcattcctgcgctaaccgctgggctagaccgtgggaggaggttgacagtagctgagaggcacatgggattagcgacagcggggaaagacacatccggacctcgcaggggctagtcggccgaagggccgcggccgcccggcggtcatttctcttcacgtccctccgcggggcgggaacgcgaaccgaaggggaactcttacaaacttttaaaatccccaaccccagccccacctgggggatggtgagaaggttggagtgcgctgcggcgcggaggagagagggaaggtggttggtgcagtgcagaaccagatttcacctaagggcgttccatgaaatgaaagcagaagtgcttgtcagtcctcttggctgggaaagccctccccag

SEQ ID NO: 506 - DNA, 642 bp, Homo sapiens
tcagtgggaatttccagccaggaagtgagagagtgagcgagacagaaagagagagaagtgcaccagcgagccggggcaggaagaggaggtttcgccaccggagcggcccggcgacgcgctgacagcttcccctgcccttcccgtcggtcgggccgccagccgccgcagccctcggcctgcacgcagccaccggccccgctcccggagcccagcgccgccgaggccgcagccgcccggccagtaaggcggcgccgccgcccggccaccgcgcgccctgcgcttccctccgcccgcgctgcggccatggcgcggcgctgactggcctggcccggccccgccgcgctcccgctcgccccgacccgcactcgggcccgcccgggctccggcctgccgccgcctcttccttctccagccggcaggcccgcgccgcttaggagggagagcccacccgcgccaggaggccgaacgcggactcgccacccgggtaagccaaactcgggcgagtgggggcccggcaggggacgcgtggcccaagtcctgccgcccagccctccgcaccccctccagccccactcggacttcctcattcctgcgctaaccgctgggctagaccgtgggaggaggttgacagtagctgagagg

SEQ ID NO: 507 - DNA, 413 bp, Homo sapiens
cccagggccacccaatcacagggccagtcatccgttgagaccctgcctccgcgcccggcagccactccgtatcttcctcgcattatcgcagggttgggccgaggcccgcgcatgcctgcagaaaacctacggccgcgaggggtcgggcctcctcctgctcctactcccgagaggctccggcaatgagaataggccccgcccccccgcgcagccaagtctacggaccaagtccgagcctgcagacaagctccgcccccacgagggcctgctccggctgacagcgtccggcagcgcggcagagccccgcccccatgcgggggcacgcttactgacaccgtccgtgcgcgcgggaagggcccagcctcgcggcccggcgtggctttgtgacgggcctctggtggcccagccccttc

SEQ ID NO: 508 - DNA, 4712 bp, Homo sapiens
atgtcatatatatatgtcaccaaaccgaacaagtgatcagagttaattaagagaagggtttgccatcacccggatctggccaactctttcgtttggaaaatgcctacaagctcagttcgtggcaccttcccaccctcactccctatccctctgcaaagaaaccagggaacttttctttggcattgtgggtactaagatgcgccaggagtcccgggtggtgcgcgagcggcggaattcgtgacatcgccaagttgcgagaggcaaactccccgattccatccaaagcctctcgcggagcctttaagagacgttgtgtgtgcgcgctgcgctagtctccccgccggggcggaaacctcgggtgcgggatgtggggagagcagaggcagcgctcgcagggccagcacgggcccatccccctctcggcggcggcggcggcggcggcgcggccaccccgtccccgcccccccaacccccggtcccccgcgtgccccgcgccgcgccggcgacgccgctcgcgctaggaccgggctgcgcccgcggccgccatggcggggccgccgcagtccggccaatgacggcgagggggcggcgcgcgcgcgcctggccaggccaaccccggctgctgccttataaggcgcgccgtcgccatggcaacgtgcgctaagttgcagcagtcgtgtcaaagttcactatatagagagctcagtgagctgatcgcggagaagccacttctgccagccccggcgcctataaatcgcattccctcccgcgcccccctttttagcatatttgatcactttgattctctgttcttttctctccgcggtgtgtgtgtgcgtgcgcgcgtgtgtgttttcttcttctcctcctcctctccccgagttgcctcctttctccgggtgccgtactgccttttttcccctctttcattctttctctccgtctttttctcccccctctgcgcacgaaggatgtgcttctaggtggtgatctgccctcctctctctcttttatcatttctcccccgccgccggcgagttgactctttccctatgtgtgtgaggcggcggcggcagcagcagcagcagcggctccggcggcggcagcagcggcagcagcgacttcagcggcggcggcggcgctagacgcagcggctccgggcccgacccggcggcttcggcggcggctccggcggcagcggcggcccgggcggcccgcagggaacggcgagcggcctccacccagcgactgcgggcggcggcggccggagagagcgaggcgcgcgccggacgcccggggcaggcggcggcggcggcggcccagcgccaggacgacgccgcgcagcgcccgacgcggaccactttcatgctgattcccccggacccgggcagcgctccggccactccgcgggccgccggcctccgccccggcctgcctggctccctgggcgcgcccgcacccggcgcctccgatctcctagtcctcctgatttcgatggctttcctgaatggctgactgtgggctgccctggacttggcccccggacagtcgcctctcctcctcctctacctcctccttcaccaccacctcctcttcctcctcctcctcctcctcctcctccgccaactcctcggctgcacaccagctctaagagcgagagtgaacgagagagggagggagagagtgagagcgagcgagatctttggagagatttttttttttgcctcctacttctgtcttgaagccagacaatcgacttcagctctccctcccctccctctttctccacgttctgctcccactcgctctcctgtccccttcccctcccctcccggcggaaagccccccgaaaccaacaaagctgagccgagagaaacaaacaaaacaaacacaccgggccagacaagccatcgacaaaactttgcaaaagcaaaaacaaaaaaggaaaaactaaccaacctcaaccaaccagcccccgagccacccggggcgccctcccgcgccctcttgcaccctcgcacacacaaaaggcggcgcgccggagcccgagacccggggagccgccgccgccccgccgccgcccgcagccaggggagcaggaagtccggacgcagcccccatagatatggcaatggtagtcagcacgtggcgcgacccccaggacgaggtgcccggctcacagggcagccaggcctcgcaggcgccgcccgtgcccggcccgccgcccggcgccccgcacacgccacagacgcccggccaagggggcccagccagcacgccagcccagacggcggccggtggccagggcggccctggcggcccgggtagcgacaagcagcagcagcagcaacacatcgagtgcgtggtgtgcggagacaagtcgagcggcaagcactacggccagttcacgtgcgagggctgcaagagcttcttcaagcgcagcgtgcggaggaacctgagctacacgtgccgcgccaaccggaactgtcccatcgaccagcaccatcgcaaccagtgccagtactgccgcctcaaaaagtgcctcaaagtgggcatgagacgggaaggtatcggcctctcatttctccttccctcgtcctgggtcccggggtcctgggtacgtttggctagcctgctctgggtaaggacaagaagccccaagctcttctcttcgtattgcagcggaaaagggttttatactagaagcgagttctgcattggaacccagaccccaaatccgcatgctttggccgactgatttccttctttactctctctttgggctgtttccatttcctttgcattgattgtgagttcactggagtctgcctttctgcaagggatggggtgtttgttgttgttgttttaaagcctagtttacttctctctctctgcccttgtttttcctgcatgttcaacatgtccctccccctcacccctttccccagcccccaccctctcaaaaaaaaaaaaaaaaaaacctgagattgtacttttgtacaggaggttcaaattacaaatggcaattttatgcacttcgccgtattaacgctgccgcccgggcagcgctcatgtgaccctccgtggattaacatcctgctaaaaaaaaaaatacctctgctttcttttttcctttcactttttgaaacgaagagagcgcgataggaagtaggaaagggtgggcgaggggccctgggcggctgctttcgctctgcgcgagttgggtctttgtgatataaaattcgccgagcgccgcgagccgtgctttgccaatggcgcgctcggcgcggggcgcgggctccgggttggggcgagccaacgccggggtttctttgtgtttctgcgagagcgactctcccggtccggagtcagataacagcctgggcccgagcctcgccggctttccccggcccttacaggccctgcccaggctccgctagtgccggccgcctgctccctgcctctcccggcttcctctctctttagccggcctctctctctccgccctctccctccgtctctttctccgagcacactgattagacagacgccagacctccgctctctgcttgtctctcactgggggggttccccgccgggctggggctggggcttcggggtttgtgggagagtcgttccggagtggccacaggccgtctggggtggaccctcgtgccttttgcaaaagcgcctcaccctccccccagactcgcccctcccgctccctctcctccaatcaataagaaatatcagctgtttagcagtaaagaagaaagatgccctcagaatgctacatcccgcccacagcgccggggaccccgaggcaaggtggccaattctgggtcctcggcggaccagccccgagcgggcctcggaggcaagtgtgcccctctggccctcagagctcgcctgggtggtggtttgaaaggaatggtgccaagagcctggcgactccagctctgtggcaggcctgcctggttcttgcgctgctcagggcctgggtcaggtgggggtcgggctggggcagaccccgccggggaacctggggactgaggctggtcattaactgtggagtgtctcctttcctccccgcagcggtgcagaggggcaggatgccgccgacccagccgacccacgggcagttcgcgctgaccaacggggatcccctcaactgccactcgtacctgtccggatatatttccctgctgttgcgcgcggagccctatcccacgtcgcgcttcggcagccaatgcatgcagcccaacaacatcatgggtatcgagaacatttgcgaactggccgcgaggatgctcttcagcgccgtcgagtgggcccggaacatccccttcttccccgacctgcagatcacggaccaggtggccctgcttcgcctcacctggagcgagctgtttgtgttgaatgcggcgcagtgctccatgcccctccacgtcgccccgctcctggccgccgccggcctgcatgcttcgcccatgtccgccgaccgggtggtcgcctttatggaccacatacggatcttccaagagcaagtggagaagctcaaggcgctgcacgttgactcagccgagtacagctgcctcaaggccatagtcctgttcacctcaggtaggaaggagccctgtcttctcgtgcccacgggctcctagcccagagctggggcccagagaacttgggagtccccagggcaaacccag

SEQ ID NO: 509 - DNA, 3476 bp, Homo sapiens
tgcctttgggaggaaacattattaagataattggcatgctgatctgggaaggtagacagctctgtcttgtaacagaggtaaggaaaagccccacagcccaaggatcaacgtcaggagggagcccaaagacctgaaatcctcctgctctaaaccgcctgatctcatttcttcttctctcctcccagataaaagtttccttcgcccgggagtcggttgttcccggctgatcccgggaagccccgcctgagccccggtccggggcgggcagctgggagccggggcgccgctgtcacccgcgcctctgcctgtcacttaccgttgcgaaaagcgccggccggggcgaggacgagggcgagcacggcgcagaggagcggcagccccctctccattctcccttctccgggtccgcaggcagacgcgggagaacgaggacgtggggggaaatgcagcaaagaggagaatctaagcgatccgaagagccccaactccgcctagagctgtacaatcctcagcccgtcttggagaaaagaaagcagcgaggcaatgcctggatccgagaggaacgcttctctttttgtgtctcaagtcgcctgcatcctgtcatttagctccggcttcctctcccttttcccacacttgttcctcttccaggagccactgcccgggccatgtctcaaaaaaaaaaaaaaaaaaaaaaaaggccgggggggggctgtgggtgggagggggaggagggaggtagggcaacacacaccaaagccaatttccaggaagaaaaaaaaaaaatccggcttgtttctggacccgttggagccgcggagctggcgcccaggggaggtccgggtgtctgcgggaaggaggggaacgagcaatgaaaagatgagcgggagacagaggagtttcaccaactgcacccccgcctcccctcctgcggcctccccccactccggagcccttccctcgcttcctcccgcccctctccccgcttgggctcgcctgtaattgcctgacaggagactgggaggaacaactttccctccgagtgctctccccgtccgtctgtctgtctttcccagagaccctgcagggatctccgcgggaaggaaggcgctgggagtctgcgccccggtcgcgtgggtgcgggcgtcggaggaaggggcgcggaggtcagtgccagccgcagggtctggccagcccacctcgcgtgctcgctctgcacccctagagggagagctgggcagctcctgcaggcgcagatcccgccgccgcgcagtgcggagcgcccgggaggtggcggctgcttctcgcaacttcaaatcccggggcccgcgccctcggcggcgaggaacgcagggtcaccccctgccactcctcatcgcgccatcctgagacctgtggcggggagcagcggggcacccctggacgcccctgcccagagctggagggcgaggaggggaaagccgggctggagtgggagccgcaccgcgaagccgggcgaggggcagcagtgtcgctgtggtgtgaagcgaagacagaccgcctgggcgggcgaggccgggctgcagccccacgcggcctccctggcccgcatcagggcgcgcggctgggtgccctctgcgcgccgcggaggcttcattgtttcccgagatttggtgcccgttcctctggggcagagggaacaccggagggtgggaggtgctgcgccttccaagcaggggggcaatcgccgcccgaccccgcccaaccttgtctgggagcccaggtgctagggaggggtgtggaaatcaggggtcgggcggcccccgtttctctgtgcatcccgagggagggaaagagtggcgggggaggggtgctcttcgctgcgccctctcagtgtccgctcccggccacagacaacgtgcacgccggccctggagctcctgccacccccacttcccacagcggctcgtgcccccagcccgctggatgccccccaggcccgtccccgagggttttccgagaagtgctgcaggaagcgtttgtagatgcgaaaagcaaggacatttctgggaagaaacaggttgcggtcaccgatcaggccccggagaagggtcagtgcccacggggcacctcccagggagtttgtgggtaaggtgaaaaggtcacacgcctcctggggattggcgtctacacacacagccaaagtacagcctgattcctacctggcacaaatagctggccaaagggaggtaggaatgctggcaaggagactctggtcaaaggttttggcggaagaaaaaggggcgggcagggagaaagcccaaggcgcgcgggccccaggccaggactgcgcgctcgcgactcgggctgggagcagtgacgtgcgcccctcggggtgcaggccgttcccgggccagtgggaggtgcacccacggagcccgcactcgccactctcttgccccgggtcaagcctgtcttctcctccccacgcgcctcgggattcccagtcgccaagttggaaactcggcccccccgagcgcctctttcctctgccagtttcctccttcttctcagctgtcggacccctgggaggcgcggggtgggaagtgggcaccgaagggctgggagttaaatgcctaagttgagcggaaggagcactgtcgaagccatgggcagccctgcgcccgggcaggcgggcaggcgggaggcgaggcgggggcgccgaccgcgcccagcgcagaccccgggggagtgagcgccgcagaggcagcctgggctctcggctactacccgcgcgcggtcgccgcctccccaggtgcagccccgccagtcccagcccccggctgcgccgggcgagcggcatcatcagatttagggtgacccggagcgacctcggtccaggctcggagggagaggcgcctctcaggacctggcaggttgacagggaaggggaaccgggagggagcggcggccggggcggcctggagtctcggaggcccgcctgcgtcctggggggacccgacctccgcgctcgctccggagcgcccgtttggatagctctgagcctcggcggggtgagcgagggggcacacgagggagacttgagctgtgttgtttttagaaaactgccgccaatctctcttcccctttccctgctgagtgtgcattttccagcctagacccgccaggccagcaaataaagccaccggatcgtgcactccggagcgactgcccccaccccgaccttgtctttcccgaagggaatggctcctccggcgcctcaacgcacagaaaacgcctgcctccagctgagcgcttttgccagctttctccaaccagtgaataagggatgcactatggactcagaaggaaatgtcatatttaagttctgcctctgatttctctgctcttagctgatctaaggtttttctagactccttagtttccactcacagtgctgggtaatggagccctaccgcactgattgagaattgccg

SEQ ID NO: 510 - DNA, 1126 bp, Homo sapiens
ttttgtatctgataaagagcatacttccatctaatacaaatatgttccccccttcagatcttctcagcattcgagagatctgtacgcgcgtggctcctcattcctcttccttggcttcccaagcccccagggcgtcgccaggaggaggtctgtgattacaaaccccttctgaaaactccccaggaagcctcccctttttccggagaatcgaagcgctacctgattccaattcccctgcaaacttcgtcctccagagtcgcccgccatcccctgctcccgctgcagaccctctacccacctggatcggcctccgaccgtaactattcggtgcgttgggcagcgcccccgcctccagcagcgcccgcacctcctctacccgaccccgggccgcggccgtggccagccagtcagccgaaggctccatgctgctccccgccgccggctccatgctgctccccgccgcccgctgcctgctctccccctctccgcagccgccgagcgcacgcggtccgccccaccctctggtgaccagccagcccctcctctttcttcctccggtgctggcggaagagccccctccgaccctgtccctcaaatcctctggagggaccgcggtatctttccaggcaaggggacgccgtgagcgagtgctcggaggaggtgctattaactccgagcacttagcgaatgtggcacccctgaagtcgccccaggttgggtctcccccgggggcaccagccggaagcagccctcgccagagccagcgttggcaaggaaggaggactgggctcctccccacctgccccccacaccgccctccggcctccctgctcccagccgcgctcccccgcctgccagcaaaggcgtgtttgagtgcgttcactctgttaaaaagaaatccgcccccgccccgtttccttcctccgcgatacaaccttcctaactgccaaattgaatcggggtgtttggtgtcatagggaaagtatggcttcttcttttaatcataagaaaaagcaaaactattttcctagttgtgagagccccaccgagaatcgaaatcacctgtacgactagaaagtgtccccctaccccctcaacccttgattttcaggagcgcggggttcac

SEQ ID NO: 511 - DNA, 745 bp, Homo sapiens
tgttcattgctttcagtacatggaaacgtaagccttatgaggatatagaatttttctactatcttattcattgttgtattcctgagtgcctatatcagtgctgggtagcaagtaagagctcgataataaatattttttgaatgagggagacaggtctgaagcctggagaatgagatgcagaagaggtgcaagacctgctgcgccctctgcaggcggcgggggggcggtgcaggtgctttaagaattaccgcgggactcggtagggggagcgtaggcgcttctcgccaagatagaagcgttcagactacaactcccagcagccacgaggagccctagggcttgatgggaacgggaaaccttctaacctttcacgtcccggctccgcgggttccgtgggtcgcccgcgaaatctgatccgggatgcggcggcccaatcggaaggtggaccgaaatcccgcgacagcaagaggcccgtagcgacccgcggtgctaaggaacacagtgctttcaaaagaattggcgtccgctgttcgcctctcctcccgggagtcttctgcctactcccagaagaggagggaagcacaggtgggtttctttagctctgcgtcggatccctgagaacttcgaagccatcctggctgaggctaatctccgctgtgcttcctctgcagtatgaagactttggagactcaaccgttagctccggactgctgtccttcagaccaggacccagctccagcccatc

SEQ ID NO: 512 - DNA, 499 bp, Homo sapiens
tggagtgggtaagatcattgcaagcatgacagcagactcgcgaaggcacaagaaaatgatttatgaaagcagatataaaatgggtattaacatatataatatttatttctatgatggcgaatttgtccgaaacacattgttttcttagagtaacctgggcctcatattctccaacgccattggaagtgaaaggaaggttgggcgggggaaaaagaaaagtgctttgtaagttcattttcgttgtcagaagagcttgtggtttaggttctccacagaggcaggaaaccttcagtcacgtggcaaataccccactgagtgcgcatgctgcacagatctgttgctgcgcatgcgcctttctgcccaccttctgtctaggcagagctctgcaaggagaggttgtgtcttcgttctttccgccatcttcgttctttccaacatcttcgttctttctcactgaccgagactcagccggtaggtctgcagagtggtcttcctggta

SEQ ID NO: 513 - DNA, 2748 bp, Homo sapiens
tgtgtaaagtgttgtaaaccttgaaattagtcctggactgaaggcactttccctcctcccagactcgcttctctctgacacatttcccctttcaatggaaagtctatggcagtcaggtccactcagctcagacttcaagtatgtgagaccacaggttcccaccttaactggtcacccattgagggacagcacaagaccccggtgctggcgccaacgctgagctgcgccctctcggttaccatggcgaccgcaggcggggcgaggcccccactcgccgcttccccgcctctgcccagaacctttcgaacgctccaattgggacgaccctttcttgcctctgtcccgccccattcggctctaagcgctttgcgattggcccgggacgagcggcctggtacccagagggcaccacctcctgacaggaagagcgggaaagtgcaccaggagctgtccgaggcgtgaagctcacgggcgcggcgggggagcacacggttggctttgcagcgagttgcccactgtcccatacacaaccccccgctcccatgactgccaaaagcatggagcaggagtcggggtgcgggcacaccgtttgtgggccggcccggtctatcccggggggctaggacgcgacgaggttggagcgaggatccggcggcacactccggagcgcggagtatcagaggccagagggaacgggccgggccccgggggcggtgccggcggcggcggcgggaggcgcagagcccaatgagcgcgcgcaccgccccgggggcggggtctgaggagcgctcccccgcccttgccagcccccgtccctccccccggcgcggggccgcagtgagtgagtggcactggcagcctgtcaatccctcggcccagcggccttccagcccggtccgcggcggctgctggctgggtgcgcggctccggcggcggcggcggcgacttctcccgccctatccatcggctgtccgcccggcgcgcggcccgccggggccctcctccgggctcagcgcccccgccgcctctgcccgcccgctcccaaactttcctcctccgccccccctcgctccccgcggcccgcccgcaggcctcctcctccgccgccgcccgccgccgccgccgctgcctcccccggggccgccggggctcggatcccgcctggccgaggcggcggcggcggccgaggagggcagtgcgcaggctggcgcggggggcggcgggcgtcccggcaactcggcgggcgctgaggagcaagttgcgcggggccgcccggcggggcgcgcggcggcgaggaggcggcgcggcggccgcgtgagggcagcgggcgccgccgcctccgcctccgccaccgccgcggagcccggcgcttccgcccgccgcttctcctcagcctcggcggcggcggaagccggagccgggcgcccgtcctcatcgcctcagccgcgggccctgccggccgggccgccccctccccgcgcgggcggggagcgcgcggccgcctccccctccccctccccctctttcttctcctccctcgtcgccgccgccgccgccgccgcctcagccttcgcctcagccgccgcccgctcccgcccgcgcgcggcgggatggacgatcaatccaggatgctgcagactctggccggggtgaacctggctggccactcggtgcaggggggcatggccctgccgcctcccccgcacggccacgaaggggcggacggcgacggcaggaagcaggacatcggcgacatcctccaccagatcatgaccatcaccgaccagagcttggacgaggcgcaagcaaagttggtgtcgtctcattaagcatcttttgtgtgtgtgcgggagccgggcccgcggccgagtcgaggcccggggtggcgcccggggctagggccgcagcccccggcggggaactttctccgaaagccggccgcccgccccggcctcggggggacttgcccgcgccccccgaagcgggcgggagtcggcaaagttgctctggggggctcgggacggactcggtgcggcgcggattccgtggcgtcctttcccccgtcctgcccctcgcagcccggccccctcccagggatttaaacctcccgccccgaccccgtgcgggccgcgccgccgggaagtgtaactttctcctccggcggggagtcccggcgccggctcccgcggccgcgggacgacctcgcgatgcgggcggccacccgggcccggcgcggggcgatgggcggttccctggcgggtccgggtgcggacggccaagttctcggagagggagggccgccttgcaaactttgccgagctgtcaccctcccgctggccgcagtcggccggcctcctctgcacaggagcggacgcgggagcccctccgcacccgtcccctcccccgggtcgccttcgcctgcccccggggcgaggctccccgcgcgggttcgcgtcgcgtctgcagtggccgaggctgctgcctgccgggcagatgggtccgccttgttccggctgcagctttcgccgccggggcttgctggctgtcgggaactagtcaactggagtttttgttgacttcccacggttcaaaagggccttccagaatggggggctggaattaaatcttgggtctaacttaaaggaaggcgccatattattccataggtgatgctaatacctttgtgtttcgttatttgttttttaggaaacatgccctgaactgtcacagaatgaaaccagcgctctt

SEQ ID NO: 514 - DNA, 594 bp, Homo sapiens
acaaagctgggttcctgctgggccccgccctgctcctcgcccccgcgactgggctgggcgcgctgtcccctagcgcagctatgtcccgagcgcgcccccacctgtgcgttaatctactgggaatgggggtggactgcgccttacctggggcggggtggggcttaaggagtggtcgagactgaggcggggtgggaggttcaggttcccggggcgccttccccaacccgccccgctttccccgtccctccacgcgcaccctgcctgtggtttccgtgcgcccccggcctgagggctctgggcggcaccttaacccggagggcctggaggtctgcacccgaccgccttgtgccaggacggtcaggtccacgccctcccccaccgtggctccctccatctgcagtatcccccacctccagcccgtcctgccctcctgttctccgtctcgcttcccgtcggtgcctccgggatctcacagccctcgcacctcttttgtgacccaggctgtttttctgcacccccctctcccctgagggcactgagattgggccattggcctgaaggtctctgggagcagcacccttccaggggaggtgg

SEQ ID NO: 515 - DNA, 621 bp, Homo sapiens
gggtctttcttcatagacaggagatgggctgttggtggaggggcgcagaaaagtcccgatcttcaggaaccccggtggtcctgggaggagaactgggaaagctctcggggtctcagcagagagaagaagccacgctaaagagtctccacaagtaccccgggatttgacccctccagccccctaacctgccctgggggacatgcgttcccgggcctcgtcttcctctagcgcggatcgctacacccgtccgagccctcctcgagggcctcaccaccgggcggggggagatggaacccgggagagaagtgcagaccgccagtcccaagaaaacactacacttcagctgctgcagccccagcaaacgccgaactcccgggaaagcaaccggtctgacctcacttcctgtcgccgagcggaagtgacgaaaaagagtgagccttgaggaattgccagggtaggaaaccggttgctaggaaactagggaaggcgtggtcttactttgggcgagtctacagggagaaaatgatgctctgacaaagatctattccccttcctgctcttctacctttcattcacgaacgctgaggtttctgacatcccctagaatctgctcagacacgc

SEQ ID NO: 516 - DNA, 2802 bp, Homo sapiens
agctgggccagggccgggacaaaggtttcccagggagggccaactcttccgtgtctctggcgggttttccttgttaaaggctcacaggttggagcctgttcgcggctcttggcctggtagggattttattagctctgctctggcaactgcaagccaggaacacaatgtcctgtgcaggggattgcccatgcagcccagctcgtgagatcgcgggatggcggggcagtgagccggtgccgctctgggagcctgagccagggcggcagtcctgtcggcctcggagagggaactgtaatctcgcaaccaggccgccgcgaggccttctgcctttgcaaagctgcgccccaccggcgccctcccaggcggcgctgccttccacattctctcctggtctacttggcctgtacctccacaacatcctccccccatccctcccagactccgtgctggctcctacccggactcgggcttccgtaaggttggtccacacagcgatttcttcgcgtgtggacatgtccgggtagcggttcctctggaaagtggcctccagctcctggagctgctggctggtaaagtgagtccgctgccgcctttgccgcttcttcttagacgggtcctcggcgcccacgtcctcattcttcccctgctggcttttatctttctctgaaaacgaaacacacacactttcccgtcagcatgcccacctgcaacgcggacgccaactggaccggcggcagaagccgtggaagagctgggctgcctggcgccggaggagggtgcgcgcggcggctccgggccgcgaggagcgctgcgcctgtggggtgtgcaggcgcaagtgtgggtgtccgcgccccatttcctcccctcccccagcgccgcacgttttatttacatgtttatctcactgcagcggcacattcacttttatagcctgtgctttcaagtatatttatacacctctgcgcagacacaccaaatctcctgggacgcgcacacgcgcgtggtttacagacccccctccccctcgcagaaagctcagatttccatgcggtttgggaaggctaggaaaagatgtggggattcggttgggcaccgaagttcgccggccctttcccaaaaaaaaaaaaaaaatgcctcttcgcgaagggcatttctgagtggtttcaggcaatttcctaacgagtggagctcctcgggagctgaaagccgagaggaaaacagggacagaggtcggcggcctctgaaggtcctcgaatcaagatgctgggatttttgtgacccaggaaacagaagggaggccagggtacgaatagagagggcggcagaattgctcgcgcccttagcgccccaggagccgggccggtcgagggagaactaaagggatgcggggtagtcaaaattccggctcccggaagttctgcggggagccaggcgaacgaccactcccaccacgcctccccccggaggggctgacttccttggggcgagagggagcgggtggcgcagagcagctgagcgggaatgtctgcagggcggcgcggcgccttacctgcggcctccgggctggaggtgtcggagatggtgtgcacctccagcctgtgcttggaggagtccagcgaccggggctgaccgggagccagaaccgaagccatggctaacggctggggatggtgacaggaagatgaggagacggccgacagcttggtccccgctgctcggtgctccaagtgaagcgggcctttcatgcagttcatggacgagggagcgcgacgctctactagtccttggctactgccccgccgagcccccgtagccgccgctgcccgctccgggtcgcgctctaggcgcggagtttccccgctgcggggagagccaggggacgcaacccccgccgagttctcaagccaagctgcccccgtctcctccggaaggctcaagcgaaaaagtccggagacggaaagtcagcgggcaaacgaagacatgggatgtgggcagaagggcaccactcagagcgtctttagggagcaggcttccaagctccaaagcgaaacaagagtgggcaaagacccccttcttctctccctccctcccccaagaacccctccaataaggaaagctaacgccgaccgcgctctgcccgccccccccccacgcggcagccctgacagagaagtgtcaagagtgacagggacaggtaggtgatattagatcccctgcggcggcagcagccgctgcagccacgacgcggccctctgagcgcaccctccgcaacgcgcacacgcacacccctcgggcggtcgaacaggagccgggccttgccgcagctcagctccaggcacccaggcgagcgacggaccagatctgcggctccgcgcttccctgttggcctaacatcttaaaaccagaggcgggcttcctggtgccgagacgtcactccgccgcggccctccccagccctctccgcctccgcctcctcccagacccttctccgggtgcgactgacgtggctccgcaccaatcaggacgccccgagccgcggtggagggactgtcctgcctgcacctatcagcagtgcggggccgggctactgcctcgccgtgcgcactgggtctacacaggcaagctcccgggaattcagctcctgcccagcccaaggcgatccggcttttagtacgaacccaaaggtgaagagatgaggctaggagtcgaaggcttgggagaagagagtggaatggtcaagaagagaaaggtacaaggatcaacaagacacccactctttgtgtctcactacatccatttcca

SEQ ID NO: 517 - DNA, 2002 bp, Homo sapiens
gtaggggagatgcaggtttggagtcaccggtgcctgtgttgttgggcaaagccaggggaaaggtcaggtcgccttgggatctggcctagcactgagtgcagaggaagagaagcttctgagatcgagaggcagcggctacaggaggaggaagaaagcaaggcggggaaaaccaaagttcaggagagcatttgcaggagggcgctgcggggtagggtctgagtcggagccccgaatcggactcgagtcctgtcgcgtggggacggagcgtgcagaggcccatgcgagggacagacacaaaggccctgggagctgcaggtcagaccactggacagcgcggccgcggacctaacgcccctgtaaggggtgctcacgcttggggggaactctccgaaagagaggaccactgaaagccaccctggcagcaggggcggaacagacagacgtgggtctggccgtcgccagaactgcctgcggaccagccccgcgctgccgggggggcgggacatgtcggctgtccatcagtgcccgcgaccaatccgcaggcagccccgcccccgcccctcccgggagagggcgcgcggaggacccgccgccgccggtgtgaggcggcccgtcctggctcccttgtccgggaagcccgcccaggtaactgagtttggccgggcattcccggaggacgcgccatcccctcacgtcccgtcccggtcgctgcatgggcggtgttcgggacgcgggggctgcgtctgtgcgggaccgcggggtagcggccgcccgtgcgagtacgcctgactgacgcgccggccgccgggcctgcggcctgtgggcggggctgccgtgcgtgacagggccgctcgtggccgccgggctgtgtccggggccgcgtgagaaagctctgcgggactgagggctgggtgggtcgaccgggaacggcgcgcgctcccgccgccatcgcgcctccgtccccgcctgcggcctgtctgggggtcgcggggcgcaggcgcggcgggccgacgggcgggggtcctccccacgggtgcgcgggcgcctttgttcccggcgccgaagcggggtggggcctcagggcagccccgccccgccgcgctgaggccccgccccgtgtccgcctgcaggagccgccgccgggtcccgctcgtctgccgcctcagcctcagccccaacctcagccgccgccgttgcgcttgctcccgggcggtcctggcctgtgccgccgccgcccccagcgtcggagccatggcgggcgccgcgtccccttgcgccaacggctgcgggcccggcgcgccctcggacgccgaggtgctgcacctctgccgcagcctcgaggtgggcaccgtcatgactttgttctactccaagaagtcgcagcgacccgagcggaagaccttccaggtcaagctggagacgcgccagatcacgtggagccggggcgccgacaagatcgagggggccagtaagtgcgcccacttcctgcctgggcccgccccgcgcgggggtcgtgggagcccggcccgactgcttgcaccccggccggccgccccagcgacttgggcaaactttcgggccctcccagactccctccgggccccgcccccgcttcgtctcgggtggtcactgggggcggggggcatccgggtcctcggtcacctgacaggacaccccccctcccccagctggggggagtgttccaggcgctttgccctgaggcctaaaaatcctcgcgggctggagacctgcggtgcaggcatcgggccccccagaccctgggagtggtggcggcgtcggcgaggggagctaaggcagtggcccccaccctgcacgggaacctggggcctgaccagacggtccccgcccactctttatccagaaagagcagtctgtgaaactctccgggcccccaggctgggctcttatttgcaaaggaatctttgggttccctaagtagaacttaggcagatgttgggtagggctggtcttggagcagagc

SEQ ID NO: 518 - DNA, 1631 bp, Homo sapiens
ctcgaatcattacatatgaaaattcagagaatagcaaaacaaaataagacgcaaaggggaacaggtctatctgcttaaatactgaaagattttacatctccaaaaagcatttgcgagatgccaagatttctcaaatgggcccctcccccgtcgtgagcctctttcctccccctaccaattctgggaagtcattagttggacgcggctgggatgctactcaccgagtttctccaccagcttaagcatcacgcctccaatgtgcacctcgccggtcactctcagggtgacatcgcggttcaggtccgtcacatggacactcagttcccacgtcccgtccgcgtagcagccatctggcatccttatcccgtccagagccatggctccttcctgcgagcgcggaggaaatggctctcgtaagcgtcactcccccaaagaaaggcgaatcccaccgaattcgcagcgccggccacgggctgggaggtggtggaggacccggggctttcggcagaaactcgggagggcggcggcggccggggctgcgggaggaccctacgccccgctcgccccgcagcgttcctcggtcccggcgccgcccgccccacaccgtccccgcctagcggaccgcggagggcttcacctgccggccggccccgagacacagcgcgggctgactccccgttcccctccccgccgcgccccctcgggtcccggcggggtcccgctccctcaccgcgcgctcctagcgctccgggcccgggactcgcgcggcaacaggcgaggggctggaggctcgcggggcggcggtgtccgcgtccggttcctcggctggcgaagcgctgcctgtggccggagcggctaatggagtcccgtcgtcgcggcccctcgcctggctccccgcgctccaccctctccccgccccctctgtccgagcctggctgggctgggcgcgctggctccctccttcgccgcccgcagagctgccctgagcgggtccggccggcctcgcctctgcccccgccctcccggcgccgcgctcgggggagggggctgcgggtcccgggcggggcggggcggcgtggggcggggcggcgcgggggtggcggggggcgcggcggggcggcaatctcggccccgcggagcgctgcccttttatggaaatgaaggacccggctcaggaattgaatccaacatccgggctctaggcggcgggccctgaggagggggagcgggggagcgcgggcggcctttcccggggcgctggtggccggcggggcgtctagagctcaggctggactggggcccccgcggcgtgtgtccgcgctggcagcagggagagggggcttgtccgtgagaaacggccccaggaaattcccagggcaaggtcgttttcggagaacaaagtcctctcgccccctccccacgcctgcagcgagacaaaaagcctcagggaggtgagaggtgccgcaccccgggagctggagagaggagtctgggacggagaaactttaaaaagtaaatgcccaaaccaactccttgaatgtcctacaaaagaataagaaaaccccgagggaggtgaaccacaaaaattgctttgcaagcaatgtccggcaccatcagcaactcctactgga

SEQ ID NO: 519 - DNA, 1648 bp, Homo sapiens
tgagccgagatcgcgccactgcactccagcctgggcgacagagcgggactccctgcccaaaaaaataaaacagaggagacgaggtctctctctgtagcccaggctgatctagaacaggatcctgggctccagcgatccgcccgcctcggcctcccccagcgctaggcccccaggcagggtccccggccccgcccctccccgcaccctccccagccgcgcgtccaccccgcagggcctggcccgggtcccgccgctctcatcgcccccgccgcgcggcaggtcccaaacgcagctgcgcggaaggcgaggcccgaaaaacctcagaaagagccgagcagggaaaggccgcgcggcccgcctcgtttccccgcaaagaagccgcgcccgtagcgcctccgcccgccccgagcccctccgtcggcggtcggggcgcgccgccctaccccagtcaggcgcggcgagtgcgggcggctgcgggaaccgggcctccctcccgcctcggcggcgccagccgaggacggccccatggcaccctccggagggccggcccgagagcctcaggagggcgcccctcacctgctggcctcgccatccgcgcctgggtccgcgcgccgccgcccgccgctcctcagagacatggagccgttggctaccacctctgcttcaggggagaaatttggcgcgctcccacccagactcgcgagagccggaaatgccgccgtccatcaagaggcgcgctccgcccctcccccgccccccgaggcgctccaattggcgccggcgcccggcgggggaggtggccggcagggaccggggaaaagtgtgtggtaggcgcgcgcggcgcaccgtggggttgggaccgggcggcgctgcgggccgcggtggggccggaaatgtcaggcggtccccactccgctctcggcgcctcgggctccgcgcccggccagcctgaggtggggtcggtgcccccggcggcacggcgctggggaggcgatggcgccggccgcgtccaggctgcgggccgaagccgggctcggggcgctgccgcggcgggcgctcgcccagtacctgctcttcctgcggctctacccggtgctcaccaaggcggccaccaggtgagcgggggcgcgggaatcggacgccgccccggccccaggtcggccgcagcggcgcggggcctggacgggagcgccgggccgggaccaggctgggggcgcgcccgggtcagaacccccggagcgggcgccctccgacccggcgctgaacttcccgcggcgacacccgcgtccgcagtcagcgattgtgaggcgccttgcggatcccgccctcccagcgcgtggagggcccgcccagacccgggggtcggcgctgaggttccacccaaggacccaacaggggccccgcgcgcccggggaagccacggcaaggggagcgggtccgcaggcgcgggtgaagacagggcgaggggagcgggtctgcggggcgcgggtgaagacagggcgaggggagcgggtctgcggggcgcgggtgaagacagggcgaggggagcgggtcttgggggcgcgggtgaagacagggcgaggggagcgggtctgcggggcgcgggtgaagacagggccaggggagcgggtctgcggggcacgggtga

SEQ ID NO: 520 - DNA, 434 bp, Homo sapiens
cctgcccttggctgggtaatctctggatttacctactgtctgtaaccccccgggctgaagagaccaccccccgggagtcaattaacttactctctggagcgcgcgttcctccctgctcccaggagtcggtttcccaaaactttctgaggcccgcgcatgctcccctcgactccccgtgtttccactctccacagaaatccacgcattcacgcccctcccctcccccgagcctgcagaggactccgccctgctttccctacattcagggctgctccttttgtcgccaatacaaacctgttgacaggtcacgcctgggaagcgggtggggtgtcccggagcggtgctgaggcgctgcaggcccggcttctggctgcgggggagctgtaccctgaagcctcgccggaactcgcgtctggggccagcagggggcacta

SEQ ID NO: 521 - DNA, 709 bp, Homo sapiens
aactcaaccaggtgcagaggcagggagatttcatacagaagacacaaactcccgctgccaagttggtgttatcttcagtttactgacaatgaaacaaaagctcccatggatttcaggaacttgcccaaggtcacagggctagtttagtcacgacgcaggccattctactgccagaaatacccccaactcccatgaccctcgcctaggactcgcaaacctggtccccgccgcccttcctcgcatcaacttctaccaggaaagcctccgggggccgctccccgccagcctccgcaccccgctccagcctgcggcctgccctccccgcagaggagcccgaggggccaggccgcgctcggcgccccatggcgcccgaaaggggacccttcgccctacccgcctgctccgcgccggggctctccgcgccctttccgcacgggccaggttcgcattcgcgcctctcgcagcccctcccagtcccctgctcgcctccgccccctcctgcccgcccggaaggggctggggcagacctcccactctccatcacttccttcttcttttcccttgctcacagcctcccgcgccctttttacctctccctcttgaaacttctccctctagaaccccctagaaccccagcggtgtctttccctccctcctcgctgcctttcagcctcccagcccccttgcctctgcctcccctaaccaagtt

SEQ ID NO: 522 - DNA, 1387 bp, Homo sapiens
tgacagcctgaggagctgaaaacgctacctggtgggcccagggtgggcggaccgggcgtttaaaggtcaaggtcaaggtggggtttggcaagtgatgcatagcgctcaaaggagaccgaagacatacaaataatattataaaattatgactatcgttatcttttttttttaagaagaaaaaaataaaaggtctcctggacggctgcggggtggaggcggccctggcaggaacgcgtgcaaaataggctgggggctgaagtcgggacggagggcaggccgagggacctgtgctgggccaccggagggaggcactgagactcgggcccagggcctccgccgcactcacttgccctcctggcagtcatagagcacgatggagtcgtcgtcgctactcgagatgaccgtctcgccgttggggctgaaatcgaagcagttaatcttgtccgagttttcgcggaacaccttagcgacgcggaagctccgcaacacgctgtcggtcagcttcatggcggcggctggggaaggcagcggcggcgcagggccggggcggggcccggcggcgagcgggcgggctgccgaggggccaacccaggcggggcgggcgccgcgccggcggctagcgggaagtcggccaacagttgggccgcctcctcctcttcttcctgcttggtcgagggtcttctgcctggactgtggagcctcgccgaccgttcgtcctcacagccacctcacggacaaccggcgcgtcgccggctcattgtgtccgccattttggggcgacaggcagaagctgagggcgaggagcctcagccgcggcgcacgccgggaaagggggcggggcgggaacggcggagcaaggcggggaggcggggtggtgacgtcagcgcggccgcggcggagtgtagtggcggggcgggctgctggcagggagcggccgagacgtgcagggccttcccgggggatcggaaggttgcggatgccttgggagccgggggcccttgcgcgggattgcaacccccgcgaagactctggcgcctcctgcaggcgtcccgatcgcaggctgcgtgggagggggtactgcagggtcgggtatgctgctggaggccggttttgggggtgactcaggtgagccgctcgcaggcaagcttgaccgctagtccaccgcagcgtccgcttagacagcttcgacctctttacttttttttttttttttggagatggagtctctcgctcaggcgcgcgccaccacgcccagctaattttgtatttttggtagagacgggggttcaccatgttggccaggctggtctcgaactcctgacctcacccgcacccggcctcgttttcttaatcgaggtagactttaggtacagtaaaaatcagcctt

SEQ ID NO: 523 - DNA, 1332 bp, Homo sapiens
gtgcaccgaggagccgggcagccaccgccacgatccagctttaggacatttccatcaccccactcagccttcctgcgcccctggttcctcatctataaatagagataatatacgcctaaccctctaggtcaccagcacattgcacccataattattatacttgttgatgccacactttcaggttttaacctttccaaagcgcagccccgccccagcgcagcccaagtccctgcacgcagaggcgccgatcacgggtgggaggtggatcccggaggccccgccccaggccccgcccctccgtgcgcccaagctcgaccgcctgaaatcggggaatcgggcctctgccggcgccctggcagcggcgcggggcgtggctccgggctggattggtggcgcctgggccccgcgagcgcctgcgcagtggtcaaggccgcgctcgcgccgaggggctgcgagagtgaccgcggctgctccagcgctgacgccgagccatggcggacgaggagcttgaggcgctgaggagacagaggctggccgagctgcaggccaaacacggggtgagcgcatcagcccccgccaggcttggccctcgcggggcgccgccctcgcccggagtgtagggcgcgcctccggggcggaggctctgggcgcctccccggggccccggtcccctgcgctgccctggcggcctcgtggccggctcagaggtggcctcgtcgctttcctcgtcgcgaggggcccaggaggctggcggcgggggccgccacgtgcgggacctgccctggcgttggggtgtggggtcccctagcctggagcgggctcgtcctggcgggcctggatccaagcacaatctcagcttttggagccagcagccggaccgaattcagtttttccggagttcagttgttatgacccaagtttagccgacaggtattgagggtccgctgtgttcccggtgctgagagcgcggcagggtcggaggaacgcttgccccgccgggatgacagggaggggccgcctgggaagcttggatggatcacacgcccggagaggcgtttctgataagggaccgggaccttagggacggagggcgcagtctttgagttatcgttgtccctaataccgaggcccgttttattttccagaaattattcctacatgtgaaacacggtttcgtaatacagggattcccctccccagggtgctctgtattccaaagccttctgaggttgtggttttagacgttcatcaaaacgttgtaagtgggaactagttcattgatttctgtgaggcccagtttcttatttggggagttgtttctaccaccgcaag

SEQ ID NO: 524 - DNA, 1483 bp, Homo sapiens
tgttcgatttccccccaaaacctatccactcgtcgtgcctcctggcccctcaggaatcaggaccctctcgggcacaccaggttcagtcctgcaaccttcgttcctagcccgagctcgccccaccttccaaccaggccccgaatggccccccatcccacctccaacccaggactcccggaggccctccctctctccaagccggggagcccggtgtcgaccctcacgcccacccccgcaataactgggcgccccgtcccgcgcggtcacctgcatcccgccgccaccgtcggccgccgcctcctcagtgcgcggccgcttgctcaggccgggatcggctccgtctcctcccgctccgggcaccggcagctccagctccgactcccgcttcatgccccgggcggcggcgccgggcccgagctgaggcggctgatgcggctgggcgccctccgccatccgcccgcgcccccctcgcctccgggagccgggcgacccgcgacaggccacctccccctgggcctcggccccgactgtcgcgacaggcgggcgcgcgcctgcccccgcccccgcgtgcgtgcgtgcgtgcgcgcgagcggactccgcgcccctccgcgcctcgcggccgccgctccttgcctgaccgcttgctccccgcccgcccgcccgccgggttgtcggcgcggggccactggcgggtcgtgatgagcactcgctcgcgcccccgcacgcacacgcgaaacccggcccggcccgccgcgccgccccgcctctcgcactcccggagctcgcccaccggccgcgctggctcacactctccctcacagcacgccggccgagggaggaagggggcggtccgggctcccgaggcgtggggagggctgtttattttggggggaggaggggcgcgaggcaggaacgagctgactggccgggatcctccgacccgccactgtggcagcaccgggaaggcggggagagagaaagagggagggagggagggaccgggatgtagaactccagcccgcgcgggaggctacggcgaggggggcggtggcggcccgcggggggggcggggccaggccccctcggcaatctccgtagtctcctcgctggctgcccgagggaggccgggaagcgatcggggaagctcgggaatctccggcacgggcctgggattgtcctggaggcacagcgcggctggagtgcggggcagcgcggggggggcggggtctgtctcctttctgggcggggccgtatcctggagcaggcggggcttgagagacccgaaggccagggagtggctcctgcttgcggtactagttgtacagagttaagtcctgagtttactcgcctgagcaccttggttcccggagagggaatgggcactctgtgagaggcaggctatttgcctgcttcccctcccgcagaagaaaaaatgtctcaaattggaaggtcgaggaatgaagccacccctctatggttcacc

SEQ ID NO: 525 - DNA, 848 bp, Homo sapiens
aagaaaacgccggcttgtgcgctcgctgcctgcctctctggctgtctgcttttgcagggctgctgggagtttttaagctctgtgagaatcctgggagttggtgatgtcagactagttgggtcatttgaaggttagcagcccgggtagggttcaccgaaagttcactcgcatatattaggcaattcaatctttcattctgtgtgacagaagtagtaggaagtgagctgttcagaggcaggagggtctattctttgccaaaggggggaccagaattcccccatgcgagctgtttgaggactgggatgccgagaacgcgagcgatccgagcagggtttgtctgggcaccgtcggggtaggatccggaacgcattcggaaggctttttgcaagcatttacttggaaggagaacttgggatctttctgggaaccccccgccccggctggattggccgagcaagcctggaaaatgcaattgaaacacagagcaccagctctgaggaactcgtcccaagccccccatctccacttcctccccctcgagtgtacaaaccctgcttcgtctgccaggacaaatcatcagggtaccactatggggtcagcgcctgtgagggatgtaagggctttttccgcagaagtattcagaagaataaaggtgtgtgggagcttttactacacctttatcttcagtaaagagacagagaacacagttccatttttaatgtttaaatttcatttcaaaaagcaggtctgtagtttgtaaccatgacaattaaaatctgtgctaatgcacggcagtctataacaattctacaagccaatcagacagtacgtgacatttcaatgagtaa

SEQ ID NO: 526 - DNA, 465 bp, Homo sapiens
gttgttttttggttgtttttttcgttttcgtaggcgcgcggggttattatttacgcgcgtattgtaggtttttgcgtacgacgttttagatgaagtcgttatagaggtcgtattacgtgtgcgtggcgggtttcgcgggttggaagcggtggttacggttagggattagttgtcgtgtggggttgtacgcggtgtttcgcgcgatgcgtagcgcgttggtacgttttagtcgggtgcggttttttttagcgcgtttagcgggtgttagttttcgtagtttaatgagtttaggttttttcgatatggttcggttgggttcgtgtttcgttggttttgggcgttagtaagcgcgggtcgggcggggttatagggcgggtttcgattttagcgttttttttaggatttagattgggcggcgggaaggagttgaggagagtcgcgtaatggaaatttgggtgtagggat

SEQ ID NO: 527 - DNA, 979 bp, Homo sapiens
cacagcctgcctgtgcctcccctgccgcccccaacaaagttttctgctcttatgttaaccgtttaaaaacaaaaagtagcaaaccaagccccagatttactaatgaaattacccacctccagggccaacgagcagacatttgccaccggttaacctaacaaccccgaattctccaatctgcagcctgggggctgccctgggagcggtggccgaggacgccctttctggcgtccgggggcaccggaccgcggctcgccgggatggaggcaggcaggcagcgtctcgggagggctctagggagcggcccggagctgctcggcggggcgcccagggcgcagcgggcagcgcttaccgagggcgcgcaggtactcctcgaaattctcgttgaccaacatcttccagtacccagtgaagtcgactggcatttcggggagtgactggagccagttggccacaagcgggcgggacggctggagactgccgggacagcggctgccggtgctacgcgggtggtgggcggcccggaaatgagcgccctccggggacagggggctctgcggggcggcgacagctggattcccagcgcgcacaaagcctgcgggaggatccattgtagcggtcgctcctccccgcttagcgagggcgggcgcaggggcgggggatgtcgaagggtcaggtttgtccaggccgcgccaccttcgtttcagacgttcagttcgtttcccctacaaaggaagaggagaccggagacgggacgtttgctttgttggccgaactcttcggctgctggggatttccttcaaacgccattaacttctcacactcagcagccactcgggagttgggaaacaatctcctggggtttgtgctggcgtgattaaggaaagaggccacttacaggcctttgtctaaagcctcgcgccggtggtggctggggtcaggaaagctgggaggttcaactacgggcgagaaaattggg

SEQ ID NO: 528 - DNA, 784 bp, Homo sapiens
ccttgctttctccctgctcctggaatctgagcacgtggcatcaagtgcgcttctacctctctactcaagcctctagcgccccatccccgcgcccacccaggtccccaggcctgccgctccactccctaagagttggtgggacttcccccaagcagttggacaacctccccttacctctccaggcaggtggtggggaaggcggccagggtgcggcacatggctgcgggcgcagggcagcacgtagtaggcaggatggtggcaggctccaggaggctccgcgtgcgccaggaagcaaaggcgcggtagcaggtgctagtcaactgcggttgggtttagcagcctgcaagcgcccagactgagcggccgctgctttaaatcccctcggaggagcggggcggggctgggcactgctgaccaatcggagggtggggctgggcgctgagaaggcggaggcggggcccgcagccagactgggccgggtgcaactttcgcggcagctggagcacgcggagctgcaccctccgcccagaggacgccgcgtgtggaaggaaaagcggcaagagaattggcacggggatgagacgtgcacacccagtttcctagggaaaagtacctgccgacctctagtaacagcaggaaagtcacgcagcggtgtcgtgttgagagagtttgcacgggggtggtgggtctgggaggcacacagggctttctttgatcgcaggcaagtctgagcctgctgggtcctgaggacaggcgcacagggtgcaaacctgggcaaaacagc

SEQ ID NO: 529 - DNA, 1961 bp, Homo sapiens
acaggatttggtcattttctgccatgaatctagggcacgtgggtgcggggaaggagtcgggcagggggatgcaatagaggggaaagggccccatttccctcctctccgtcttcggagctgcgatcccaccctcagtccaagggcttaaacatctgcttttcggaactggaagcgcagcacaaaccttgcttttcaaaggcgctggggtctctcggcggccccatctcgggaagaggaactaacgataactacagcacgaagcacaccggccgagtctccggcgggaaaggcacccgcgcgcgcggacacaggacggacacagggaggagcggagcgggtcacttggtcgccacccccgaagtgacggccgcggcggcggcggcagcggcggcggcggcggcggctgcggtggtccctgcgggcactgcggcgggggtggctgcggtggtgctggcggtggtccggcgctgttccatgccgttgggcaggaagccggcgatgatgggcaccggccagatgagggcggccacgctccacaccacgatgaccagggtcatgaagcaggccaccagcgtggactcaatgggcttacggttcagccgccagcccggctcgccctgcagctcctccaggctgaagtctaggcagcagaaatgcagcggctcgccctgcagccacagcggcccgggcggctcggccgcggggtaggctggcagcgcggtgggcgccccggcggtggcggcggcggcgctgggggcggcggcgggccggaggcggccggtgcggcgaccgcgcacccagccgcagcccacgcagaggcgcaggtcctgctgcgcgccgcccgccgccagccccaggtgctcgatgagcagcggcggcccgacgcggtggaaggcggcggcgaagaaagcgcggccgtgcgcgttggtggagaagagcagtaggtcgcactggaagccccgcgggcggccccagcgcacgagcagcgagctcagcatctgccgctgcacgctgatgttgcacaggcgccgccgccgccgccgcctcctccgggcccgggcgctcggggggcccgggggccgccgaggccagcagccgcggggcgatgggctcgccgcggaggacgcgttgactgaagcattggagggcaccccgaggaggccgggcgcgtccgcggccccgccgcggacgggcggcagcgggcaggcggcggccaggagcgcggcgagcaggggcagcatggcctgcggcgggcgcctacgcgcgcgaggccggcggcggttgcatggcgagcgggcgacgggccggcgagctcacggtgggaccgcgggagccggcggccgggcgcagtgcgggcgcgccgggcgcctgccaagcccgcaggggcggcgcggaggcggtgacggcggctggctcggcgcggggatccctccagacccggttgcgtttggggttccccgccgcccccggcgccggggcccttgggctgccgcgctgctctgcggggccgggtgctatcccagagcaggattccccggctcggctccgcctcccgcgtgccccgccccgccccgccctccgctcgccaggctcggggactcaggggagggcggggttccacgcgccggggctgggagaggaatcgccggggcggggctgggtgcgggggcgcggcggagagcggatcgcccggggtgcgtgcgggcgggcggggaagcctgcggacgcgcccgggcgcccccgcgctccgggtgaatcgcgttctcagagggaggctccagggcccggagaccatccttttgtcaggcctgaggggagcttcgctgtctcgccttagtgctaccgaacctcctttgttttacagaaggggaaactgaggtcgagagaggggaaagggcctcgctaggccaactcatgagccagaaacaagtccgcgccactagctctttctgcctttttac

SEQ ID NO: 530 - DNA, 244 bp, Homo sapiens
cctctcccaggcctggagggtttctggggaaggaaggaggcccggcgggctgtcccactgcgaacgatcggaggggggaagccagactggacgagcccaaagataaataaaggggttggctttactttttgaaagtcttgggaagaaaaaagaaataaggaaattcactatactgtaaattaccagctagtgggatggaaatcgaatctactaatttggggaagtgggaaaattccttgtagcc

SEQ ID NO: 531 - DNA, 3878 bp, Homo sapiens
cccagccgccggcccttcctgccctgcccttttctcacggcagctgtgagaggtttaggggaaaaccgaggcgttttcgtttcatctcgctgcccccttaaaaaaatgaaaatgaaacagtcgcctactccctggcataaagaaaaaggtcctctaaatggctgggggctgccagggttaggggtcccccaatctcaactcgccattcgggacgcataatatccccgagcaaacgtctggagagcagtgccccgatcccggcctagcgccgtccggtaaaatttcggaagcccgagggtgtgagcaggaagcttttgcgaagcggcgcgggaggaggggtgctggaggcggagggtaggccctttcaccgttcgcaccccacccgcggtgtccttgcccctgtcccgggatcctcttctccgttacccgcagggctgtatctgagcgatccgggttaggggggcgcaaaaccccatccgcccatttccgcaccaacgtctctacgcaaggcgccccaaaacccaggtggagcggggcaaccccgttaaaagtcattcctgcagggcgcatccaaaacggaacgccgaggtcccggagccgagcgcgcagccagactgaaccgggtgcccgggtgtcgccgcggcgtctcgggcacctcccatccccactgctcccgaggctctggctcccgcagctcagacgcccggagccccagggccggcgccctcccgccccgggtcccgcactcaccttgaaggcgacgggcagcgtcttgttgcagcgccagtgcgagggcagcacggagcagaggaagttggggctgtcggtgcgcacgagctcgcctgcgtggtccgccagcacgtccaccatcgagcgcacctcgggccgggcgcgccctccgggccccacggccgcctgcgcgctcagcgcgccgctgttctcgcccatcttgccgccgccgccgccgcaggggaaggccggggagggaggtgtgaagcggcggctggtgcttgggtctacgggaatacgcataacagcggccgtcagggcgccgggcaggcggagacggcgcggcttcccccgggggcggccggcgcgggcgcctcctcggccgccgctgccgcgagaagcgggaaagcagaagcggcggggcccgggcctcagggcgcagggggcggcgcccggccactactcgccagggcccgcccgctgcgaggcctcgctggcccgacggccgcccgcagcctgcccggctagtcccgcatcctcggcgcgcggccccgcgtgcggccgcccctcgtggctgtcccggctgcctgggccgcggcggggcccgcgcggggctgtgccgctgccgccgcctcccgccccgaagctcgcccgcggccgccccgactccgcggccgcagccccagaacaaatcctccagaatcaagtggcggggccgcggccgcccgcgcggggttagtacccccggggcccgcggggcggggctggcggagcgacgcgtcgcacagccaatcggcggagcccccatcgcgggcacctcggtggcgttcgcggggaggaacggggcctgccggaggccgcccaacggggaggggcggaaggcgccaccccgcggaggaggccccagtgccacagcccagggcccccgagagctctgggagcccggggcaaatgctagaaatttgcttagaacgtccgggtcccacggaaggcgcccttgccgccctctctcgggtcgtagctccctgacgctggggcgcaaccccttcgctcctcctccccgctggccgcggccgggcttccccagctcttgctgcttcgggcctgtgacttctgcaaccccgggctgggggccgcggggtctcagggccggtgacgccgcactgggagccgccccaaagaggttactcacctccctcgtcccgcacattattctgacccaagagcctccaccccacacgggattttgcgcgtcgtccacgcccggccggcggcctttgctgctcccagccctgcgcggctttggtcccagcctcggtggcccctgtgccaaaccggggacaggcggaagggagtctcctagggaccctaagtagcctggggccaacaacccctttcctctctgctctcccctcaaaacaagtttcaggatcttgcaggcctcgcggcgtcgttcttcgttgtggcggcctgtggctctttgaaaaacacgacgaggcctgcaaaatgcgtttttctttttttcctttacgcatgtaaccacggtcctgcatcgtgaaacggtacgcgcgtcggtggcaaaagaaaaacagcagtggctgcaaagctaagggccctcgctttcagaggagagaattttctttctccatgcgggtggaaagtggcctctgcgggtccaaccccacttcttcttgggcccgtgcgctccggctgcgccgcagggaccgcggacagcttcgccaaggcactgcctgcccgcccggctccgggtccccgctcccactcccagccgcgtggcccaacctctcctgggcttcactgcaaatcaccccttcctctcccgcctcctaagtctgtcgagcagacctaggggccggctacagttgggagggcaacgggaaagatcaagccacaatcattccgaattatcgccccagacacctccctagactctggggaacgaacgcgtgctgagcctccccgccgctttggagacggggctagattttcgttgcctccggctctcgacaggtgcaaaacaatgaattccaagcctcggaagcaaagaagcttaggatccgacggtggccgcaagatctcatcatggatctgacccctgctcagcgcgcgccatttcgtcgttgccaaacgaaatcaagccccgcgtgcgctccaggggcgaaggactctggactcaccccgaccaccgggagagctggcccctacccacctcgggacctcacagcacgccctcaggccgtgtcgaaaggaaggacggcaaaggtcccttactgaaccttttaagagagcctgcgcctggcagttgtcgattgcggacccaggcccgcgcgccctcggacgcgctggcacgagcagcagaactagaggaaagcgagtgatccagcctgggcgctcccacctccgggaacgtctccgagaaggcgcagcgcgtcgtggccaggtagggccctggccgggggcgggcaacacgtgctgccctcgagcaggttgcgggaccatgacccgctgtttcaggtggtggtaaattccatttgtcgaatggtttcggtttgcaccgtgccctttgcttgttcctccgcctgatttctccctctccgcttacgatgggttcacagacaagtttccagagaatgagggactcttgtgggccctggcacctggcgcagggcccggcacggctccggctctccgtagggcgctggctccccgtgggcaccagatccaagggaccagggcggcggggggagggggggcgggtgcaggcccttgggtccccagaccaaggtcgcggggccgcctggcaggcacagtggcgggagccgccgctagttggcgcccgcgccctgccagccgcggaggtgcgggcccggccgggctacagatgcgcgccagctgcggccccgggtgcaggcgcggcgaccgcccccgaggagctgccctttccttgccatccatgcggccaggtctcagacaaaccgatggctttgtgtcaaaccaaggccgccttcctcacctctgataagatggacgccttctgtcttcgcgttttcaggcacccggggaagacccacagaacaggctagcttgttcccaatttccacctgcttcctccccatcccggaccgaca

SEQ ID NO: 532 - DNA, 1115 bp, Homo sapiens
gcttgggacatggttatcagcgtcagatatttccttaaactagcgtgaagtcgactttaaccttccagcagtgtgagtgagggccctgaccctagaatcaacgcccaaagagctgtggcgctacagcgggcggggacatggggtgcgcatgcgtgcgggggggtacgcgtgcgtgcaagtgcgcgtgcgcgcgcaggaccgctgaggcgggagccccgaatgcggttctcgcttgctgcgtggcggtaaaggaccgcaggtgtcgtaaaagggccgcagtggcagcgtcctggccgacggctagtagcccattttggataccgtcctcgctgcggaaagttggggcaacctgttgctagtctggtcgttggtgacagcgaggcttccgcgctcgctgctggtgagcagccccggcgtgccccgcgggctggaagaggcggcggcgtgatgcggcccgtggacgcggacgaggcgcgggagccccgcgaggagccgggcagcccgctgagccccgcgccccgcgccggccgcgagaacctggcctccctggagcgcgagcgcgcccgggcgcactggcgggcccgcaggaagctgctggagatccagagcctgctcgacgccatcaagagtgaggtggaggcagaggagcggggcgcccgggccccagcaccccgcccgcgtgcggaggctgaggagcgggtggctcggctgtgcgccgaagcagagaggaaggctgcggaggcggcgcggatgggcaggcggatcgtggagctgcaccagcggatcgccggctgcgagtgctgctgagccggcgaggccgcgcgggtctggagcggagcgcggcggggagtgtcccgcgtggaaggcgctgggtaggcagggaggggagcgcagagccgtgccacgctctccgcggagttggtttcattcttttttcataggtaattagagaaaataatttgatatgttgttgtaaagagtcatgaccactggaagtattttcaggacgtagacctggtgggtcactacgtggggggagagaggtgtgaagcggaggtacatgctggaaacgtgtgaatgttgttacgagattggagcgtttgcgatgaccttcc

SEQ ID NO: 533 - DNA, 907 bp, Homo sapiens
gccacaaagtgctccaaatatccacttgcaggtcctccaaaaagagtgtttcaaacgtgaactaccaaaggaaggcttagctctggactttgaatgccaacctcagaaggatatttctgcgaaagcttctgtttagttaggcgacgttatcccgtttccaacgaaatcctcagagaggtccaaatatccacctgcagagtctacaaaaagtgtgtttcaaaactgctccacccaaaggaatgttcagctctgtgagttgaactcaatcatcccaaagtattttctgagaatgcttctgtccagtttttacatgaagctgtttcctttactaccgtaggcctcatagcgttccaaatctccacttgcagatgctacgaaaggagcgtttcaacctgaactcacaagggaaggttcacctctgtcagttgaatgtcaacatcacaaagaagttctgagaatgttcctcttcagttacgtgaggtttttcccgtttccaacgaaattctcagagaagtcccaatatccacttgcatatcctacaaaacgtgtgttttgaaaatgctccatcaaaagaactgctctgctctgtgagttaaactcaatcatcgcaaagaattttctgagaatgcttctgtcttgtttttagatgaagttctttcctttactacgacaggcctcaaagaggtccaaatctccacttgcagattctgcagaaggagtgtttcaaacctgaactgtcagagaaaggttcaacactgtgagttgaatgcaagcatcacgaagaaggttctgagaatgcttctgtttacgtaggtgagttctctcccgtatccaacgaaatcatcagagcggtccgaatctccacttgcagattctacacaaagtgtgtttggaaactgctccatcc

SEQ ID NO: 534 - DNA, 1283 bp, Homo sapiens
ctgtcacacacacacacacacacacacacacaccgtcttcacaatgcacatagaaacgtcctgatcccatttgtgtagatcaccaaaaagggcttcgccaccatccccgcaccaaatttcaacacacacgtccactccctttctgagacaaaacaacccctctcctctcctccctgtgccgaccccactggctagaagacgtgggaagcgcggggagggaggataagggctctgaatgcttctgtccccaccggctcaccgttccctcgcccccgccccgacagcattatcgccgccttcccgctctttacctgccaacaggttcctaatttcctcagggagggggtagggagaggaggtgctgctggggttgggcatgttagggagcgcagggcgtgcggggaaaggacctgcgctgaaaaggtgaccgacggggtggggctgcggctgcgacctagactcaggctagcggcccggattaagaacagcggggctacgagtcgggacactgccgggccggggctcacaacaaggaagtcactgaatctccagcgagctgcagctggactgtcggcccagccccgcccagagggccggggcggggagatgggtgggaagggacacgaagggcctgaggggtccaactgcgcatgtgtattcctcggttttcccggccccagaagaaagagcctgtgggcaagccacgcccaccgctacgccgggaagcaggaggaggagccactggtgggaagggggcggactgaggctgcgtcacctgatgctgcgtcacctgatccagcgtccggagcgccttacagccattttcggtactggtgccatcacaactgctgatcgtttctgcaggattccagaaaataatgtatccatgatacagagtgtatacaagttttgcgattacgccttctgcgtaacagtcagtcccaccgcagtttaacccacaaggaattttctcttgtcctaaactattatggccttctttgtccgattgaacggaggtaacgaagtctcgtcttccgccagtcgtccgggcgaagccagtctggagccgctccggagcgcgcgttgtgattggctcttacattacttttctacctaattcgtatccttggggtgacagattttccccactacaaggaagactgggaagttctaagcaccgtcctttggcaggaaaaaacaaacaaaacaaaacaaaaaaacagaaggccgaatagacattggcaccactgtccatctacaagcatcaaaaataaaattgctgg

SEQ ID NO: 535 - DNA, 362 bp, Homo sapiens
ctggtgacagccaggtaggtggaagtttgcacctcgccggctgcattgcgggcgaagcactggaacatgccggtatcatcgggcaccaggccgctgatctgcaggcccccgtcgttgcgctgccggaagcgggtcaacttctccacctccaccacggctgcgtccttgtaccaggtgatggagggcggcggcacacctgtgggcaagacgtgggcccaccgtcaacctgcctcctgcctcctctccgcctaggaggggtgcttgggaggaggccgggcccggaaatctgcgagggggtccctggtgaatcttggtgtctagaaccttctcagaggtgccactttgggtctagggagaggagg

SEQ ID NO: 536 - DNA, 1029 bp, Homo sapiens
taaatcagctgggcgtggtggtgcgcgcctgtaatcccacctactcaggaggatgaggcaggagaatcgcttgaacccgggaggcggaggtggcaatgagccgagatcgcgtcactgcactccagtctgggcgacatatggaggttttgtcttgtctcaaaaaaaaaaaaaaaaaaaaacttggggaagtgtgctaaaccgggtttgcgtaggtggaaggggcggccgggctctttaaggtgggtcccgccccgtaggcctcccctcgcgccagcccctcccatcccggaaaggctcagcccaagcccggccccgcccgccctcggccccgcccatggcagagcgcagtcacgtgacccgggctgcggggcgcagcattgtgcggtcatggtgggccagatgtactgctaccccggcagccacctggcccgggcgctgacgcgggcgctggcgctggccctggtgctggccctgctggtcgggccgttcctgagcggcctggcgggggcgatcccagcgccggggggccgctgggcgcgcgatgggcaggtccctccagcctcccgcagccgctcggtgctcctggacgtctcggcgggccagctgcttatggtggacggacgccaccctgacgccgtggcctgggccaacctcaccaacgccatccgcgagactgggtaagggttggcttatccccacgcggggccatcgggggagggggatgcgtgggcgccggacctcgcctgttcccgggaccggcctcttgccgggtgggaacgcccgtttctctgggggtcagctactcccgaggctgcagcaccgccctgcgggccaagtaccctgtttgggagctggctactgcggcatccctcacctcatggggccagtcccttgccttcagccagtggtgacagtacttctgtccccaagaagccagttccctaccaccgaggccgcagtgcctgcatctggatcaggtccatggctggtgggtaaattctatcctcacggggacctgctcccttccct

SEQ ID NO: 537 - DNA, 559 bp, Homo sapiens
agggaagaagtgaccctggctgatgggcccacagtcccgggtgcccctaaatgctaagagacaggtccaaatacaaagacgagcatttggagacacaggaacacatacgtgcgcaggcttacacaagcacatgcacagccacatagccacacactacacatacacaagcacttatgcacatgtccacacaggcgtagatactctagcaagcacatactcacagaggcgcgcacacacacacacacacacacacaggcgtagatactctggtaggcacatacagaggcgcgcacacacacacacgcacaaatacacacaagcacactcacaggcacattcacacacgtggaaattcacacacaggcacagatacacactcatgcaggcacgtgcacgagcaacacgcaggtatacaggaacacacacatgcccagacacacaaatccacacacacccaggcacccactaaagagcacgagctggtcaggggcagcggctcacacctgtgatcccagcactttgggaggccgacacgggtggatcacctgaggtcaggagt

SEQ ID NO: 538 - DNA, 1558 bp, Homo sapiens
aaagcaaatgtccccttgaggagagaaggtgctagctgatctggcctctctaaggtatggaactataaggagagtctgtgggagctgctggcctgaaataagggctggcgattgaggcgatcacggggagtcgttgaatggctggagagagctgtgtggctgggctgcccccgacctggcctaggctggaggctgagcccgcgggaaggtccggcggtgccagccgtcccaggggctgcaccgccacctgctgagcagcccgggcagtcaggggtcctgcgccgcggggcccacgcacgggaccaggggcacggccggcggcgagtgggaatgagggacgcggaagggggtgctcaggtgagcgcaggctcgttgctccctggcgctgcagcccttctcgccagggagttgaatgcccgggtaaacaccaggcgcgatgtgtgcggtgtattcacagagagacaaagccagggcggggcgggggcgccgcccgcgaagcgggggcgggggcgaggggtgcagactagtctcccccggcgcggggtgcccaccgcctgggaaccctgagcccggcccacggacaggtggagcgggggcggggcggtggctgggcgggtcccgccccgggggtggggctgggcgggccgggcggggcggggctgcgctatgcaaatgtcgcccacgggcggccaattgccggcgctccccgcgcggctctgagcgccccgtcccgccggcggccgcgagaccagagcgagcgaacgaaccgcggcggtccggagagccccgagcgcagcgcaggacctgggtacgccgcgaggaaccgtgcagcccagcgcggccgcccggcccgggtccagcagccaggagagcgcagcgcttcgaagccgagtgcgcgccaccgcccgcgccccgcgctggggaatgcgccctcggcgcgccggccagggggcgcccgcagcccaccccaggggaggcggccccgagcgcccctgagccttcccatggcccgggctggggcccgggccctcggctgctgacgcgcccgaagcccgcggaaccggttaagccgcggccgcggcgccgatcccggctgaggcgcagcggcgagaggtcgcgggcagggccatggccccggggggccgctagcgcggaccggcccaacgggagccgctccgtgccgccgccgccgcccgggcgcccaggccccgccgctgcggaagaggtgagtgcagcgggaaccgggagggagcgggcaggcggccgggccaccccgcgacccctctgggacccgcggcactgcaactccgcagaagtgtccggggagcgggtctcgtcgagccggggggctgcccgcggacataggggcaacaaggctggggtgggattcttacctgtcctggaggcccggaccccttacctacggggcggcgtatggatgtgtggatgatgtggcctgcggggtcacccatgtgcaaagcaactttttcctgcaaggttctgggtgggtgctctcatacacccccaccttcacgcttctggagcgggagttccgatc

SEQ ID NO: 539 - DNA, 1066 bp, Homo sapiens
gggaggctgaggcagaaagttgcttgaacccgggagtcagaggttgcagtgagctgagatcacgccactgcactccagcctgggcaacaaagcaagactctgtctcaaaataaaaataaaaaataaaaagaaataaaaaagaaatataccaaaatgttagctggggtcttctctgggtagtaaagtgctgggggatattttccaaagtccttctttacattctctgagtttttccatgttcttcaatgagtatttaataagcagataaaaactaatacaacaaaggattttttctgtgtgcttttttgacctttggaggaagagattagagctagtcccataaccaggttatttgagtaggtctaacaagcccgtattaccagaaattatcatctggtcatttccagtccgagaacagaacacttggttgtcctggcatttcccaagcagggggaggagttctctgcaggaataaataagcctcagcattcatgaaaatccactactccagacagacggctttggaatccaccagctacatccagctccctgaggcagagttgagaacacgttgcttgtgcctttccatgctgactatcagactctgcgcccctgaggctacctgtgccccaaagacaagccccttgagctcccatgcaggctcagctccaccccaagatctgggctccttctgccttcagctgcagcagcctgcccaggactacctaagcttgtggccaagctcacccagacctcaggcccagggttgggaggggaccctgctgctctaaggccaattcctgccccccaggcttctgggtgcccactttcagtttctctccttccacccccagggcccaggctccacctggctctttccagagctcctagccctgaccacatctggcacagcccccggactccttgctcctgcagctgtgcagggctttatcatgcgacatccttcttctgccctttagcacccccaatttgggggaagtagctactttctgggtttccagatggagcccctcaactgcctcaccagttcctagccctggctctg

SEQ ID NO: 540 - DNA, 1066 bp, Homo sapiens; identical to SEQ ID NO: 539

SEQ ID NO: 541 - DNA, 490 bp, Homo sapiens
gctgtggggtggggcacacttggttgggggcggacgggggtttaggagaacggcgaggagcgggcgcagaagggtggcggggccgccgggtcgttgcgcggcagttgcgtccggcctcttgcgcgcggcgcccgggaaggggcggggccgaggcgggcaaggtggcgggcccccgcccctggccccgcccccgcagcccgcccgcgagcctcgccccgcctcctcgccgggcccgccccgccccctcgccgggccgtgctcttgctcccgccgcctggcagcctcacgctcggctccagcggccaagagccggagaaagtcctgctggtgggcggccgcggggctgagggcgtccggcatcccggggccgctccggcccgggcggcgagagtgcccggcggtccatgcatccgccgccgcccgccgccgcgatggatttcagtcagaacagcctgttcggttacatggaggacctgcaggagctcaccat

SEQ ID NO: 542 - DNA, 2098 bp, Homo sapiens
cggtttagcagggcgcctggagtgaggtgactgcggaggctcgcagacgttaggtctgcctaaatccgaagcttccaccctcctctgcctctgtgacttgctgcgtgactttggagctttcggaaactcagtttccctgtcttaagccctctgctcctcttgctttcccgctccagcaagtgagagtggactgggttgccgtgccgggcgggtgtgggtcgccgggcaactccggagtctccccttccccatcggcccccagcagaagtcccagctcccggcgttttctgtctcgtggcgagttggggcggagggagcggcgggaaggtgccgggtgggcaaggtcggagctgcgtcaccgagaggccggcaaggccagggagaaaaggccccgctgtgatttggggaagggccgggggcccataagtcacgtgctcggggcggttgtgcaggagcggactgtcctctgggaacatagaagcgcccccgactagagtaggggcggggagggagcgccggaggaggcaggttctgggccaaggggatgggggtgggggggaggtagggagcccgcggacaaaggaggcggccggcggcccagctgttttgaaaaatgcttcctgtttctttaaaggcgctcgcggctcgggcggcccgggctggggaggcggtggcggcgggagctgcctcctctccgggcggcggcgctgactgatctcgcgaactgggcttctgtgtaaactgaggctaaacaaacacagatggagcgcagcgggcgttctccagaaatgctggcaaggaggcagcgacttcagatgacactctgagcgctccgggaacggacagcccggcggcttcccgaagccggcggcgcagctgcccggggcgagggggagaaagggagagagggagggggagggcgggcgaagcgggagagccagagactcctcggcgctgagcgcggcggcggcccgggcagccccacgcccctgcctcgcgcgccgcccgcgccatgaagcacatcccggtcctcgaggacgggccgtggaagaccgtgtgcgtgaaggagctgaacggccttaagaagctcaagcggaaaggcaaggagccggcgcggcgcgcgaacggctataaaactttccgactggacttggaagcgcccgagccccgcgccgtagccaccaacgggctgcgggacaggacccatcggctgcagccggtcccggtaccggtgccggtgccagtcccagtggcgccggccgttcccccaagagggggcacggacacagccggggagcgcgggggctctcgggcgcccgaggtctccgacgcgcggaaacgctgcttcgccctaggcgcagtggggccaggactccccacgccgccgccgccgccgcctcctgcgccccagagccaggcacctgggggcccagaggcacagcctttccgggagccgggtctgcgtcctcgcatcttgctgtgcgcaccgcccgcgcgccccgcgccgtcagcacccccagcaccgccagcgcccccggagtccactgtgcgccctgcgcccccgacgcgccccggggaaagttcctactcgtcaatttcacacgtaatttacaataaccaccaggattcctccgcgtcgcctaggaaacgaccgggcgaagcgactgccgcctcctccgagatcaaagccctgcagcagacccggaggctcctggcgaacgccagggagcggacgcgggtgcacaccatcagcgcagccttcgaggcgctcaggaagcaggtacccgctcgccgccgcacgccctcactgcgccgggggacgactgcgggaatgggtgggcgagtggccggggcgggatagaggtgtgtttaaggggcaagctgccccgcccggtccgaccctggtgcccagacaggtgtgggcagtcctagggtatggatcatagttttcaaagtggctataagagttggttatctctggcccgagcaccccccgccctcaacccccgcctcgggggtaacgctgtctgcgcttctgacttcagcctccatagttcacttttatggcagcgagtatttgcctgccggagtcccctgg

SEQ ID NO: 543 - DNA, 1700 bp, Homo sapiens
agactagggggaggaaaggaggtggaaagaaacaatgccctacacacaggaggggctccacaaacaatttgttgaaaagaaatcgctctgcggacgctacaaagatgccctgcaggacaagcccacttacacagaaaaaaagaaatcctatctagggagttgttggtccccagagtccccatccctcattccccaaccccgtggcgtgcgcgcttcccgctagacttcagcctggggctctttcctggcccccagatcccccgcccctccggttgcgctccccgcctggccccgagggtaaacctgcctccgcggaacacatctccgcgagactgggccaaagctttggggcctgcactcctcgcaaaggagagcctccgagaccggaggttcatctcaacccccgcgttcgagcccgggcctggagaaaagtgccctcccctcggcctcggcctcgtcaccgcgggggactggcgggaggagtaggggaggccggtgggacacctcgcaccaggccgcccacctacaggggcgtccgggctaggggagcagacggagagcgggggcggctcctgactcctccgcgcgccggggccggggccgggccgggggctcggcccgctcagccaatgggcgccgcgctcgggccccctcccccgcaccgggaggggccgaggctgggctgctgctgccgaggtcgtgtgcgacgtgccgctcccgggccctgtgtccccagccccagcagggccgcgtggacggcgcggcgcccccggaaggaggccatgtgctcagtgccggcgccaactacgcacgcggcgccctccggcccgctagatccgaagcccatccccactgcggccagcggcccggcggggcgctgcgctcgggacccgcaccgagccaggctcggagaggcgcgcggcccgccccgggcgcacagcgcagcggggcggcgggggaggccctggccggcgtaaggcgggcaggagtctgcgcctttgttcctggcgggagggcccgcgggcgcgcgactcaccttgctgctgggctccatggcagcgctgcgctggtggctctggctgcgccgggtacgcgggtggcgacgggcgtgcgagcggcgctctcccgctcaggctcgtgctccggtccggggactcccactgcgactctgactccgacccccgtcgtttggtctcctgctccctggcgtgctcactcggggacccgcgactagcgaccggcacgctcgctgttgctacctcttgcctcttgccagagagcgcgcggaccgtagcgtttataggaccccggccattggctggcgacgccggtgtgtcaggggtgtgtgggcaggacctcggggcggggcctggggcccagcctgtcctgggcggcgctctgcggggagggggtgaggggagtggccggggccggggccccccctaaccactgcctcgctcaggctgccgatcggctcgttctctctgcgttgggaccgcggagggcattcgctgtgtgactcagggcaagtgtgcgcacctctctgagcctccgcggccttctctagcaaaatggtggagccggtacccggctgtaaggcaagctgggatgcgcggagtagcgggtggaaagctggactggagttctcactcggccgctgggtgacttcggtgcactagggatagtaacagtaccacctc

SEQ ID NO: 544 - DNA, 1309 bp, Homo sapiens
acgatcacacacgcgcgcgcacactcacgagccgagcctctcgcggatctcgtagacgctgctgatgtcctccgtgtgtttcttcagcagcagcatgtctgcaggggcaggggagaggtgatgggggtctctccccgccccgccgggggcaagggcagcttcctcctccaccccctgccaggcaggcctcagccattgccgctgcgcgctcgaccctcccctcccccgtccaggtccacacccccagacggacgcctcccagcgaggccaccgcatacacgcctccacatcccaggcctacagctgctatcccacgcaccctcccccgcaccccgaaggcgaccagcgcctggatgctgcctctctgcctccacacatccggccgctttccagaaggctccatgctcacccacgccgtccctcccccatccacacatctacagcgcccaagcccccacccccgcctagacccgccacccatggcacagcgcccacaggcacccgctgcccaagccaccaagccgcccgggatgcacctgcgacccccgcgcgcagccacctgccaagaccgcggccacccacgcggccgcagacgcagcccagccctcggcgccgcggagcagggggacctggcagcctgggccgcccgcgtcccgagctgcgtggaccgagcgcgtgcgggcagggggcgcgccaggcgtgacaggtgtgctgcggacgcgcaaggctgcggcggccgcgggccggatccggtgcgcccccgccccgcgcgtccccgcgccccggagcaggtgcagacctggagcgggtgccgcagcgtcgtgccgcgcagtggccgcagcgccaagccccccgcccgcgccccgccggccccgccccctccgcgcacgcccccgccagccgcccaagcacgcagtggcccagcgcggccgcggcgccgcagggcaccttgtaaggcaatcgccgggatccggctcgggtgccggccgccaccgcccagcccactgctcgctcgcgccgcctcccggagctgcggcccggctgggccggccccagggcctttgcaccaccggtggctgtctcccggagagctcttcggccacaggtcggcaaggctctctgccttccagtctctgccttcaccgcctctccgaggacaggctgacccggccacttgcccgaaagcagcctccatgcctagctccaggaccctgcaaccctgcaccacagctctgccaccctgggctcccggcactcactgggctccctctggctccaccattaatccctcaccctcgctttcttctct

(19) United States
(12) Patent Application Publication
(10) Pub. No.: US 2009/0317801 A1
(43) Pub. Date: Dec. 24, 2009

(54) METHODS AND COMPOSITIONS FOR DISEASE PROGNOSIS BASED ON NUCLEIC ACID METHYLATION

(75) Inventors: Dirk J. Van Den Boom, La Jolla, CA (US); Mathias Ehrich, San Diego, CA (US)

Correspondence Address: GRANT ANDERSON LLP;C/O PORTFOLIOIP, PO BOX 52050, MINNEAPOLIS, MN, 55402, US

(73) Assignee: Sequenom, Inc., San Diego, CA (US)

(21) Appl. No.: 11/997,402

(22) PCT Filed: Aug. 2, 2006
(86) PCT No.: PCT/US06/30256

§ 371 Date: Oct. 14, 2008

Related U.S. Application Data
(60) Provisional application No. 60/705,068, filed on Aug. 2, 2005.
(60) Provisional application No. 60/705,069, filed on Aug. 3, 2005.

(51) Int. Cl. C12Q 1/68

(52) U.S. Cl. 435/6

(57) ABSTRACT

A large scale DNA methylation study was performed in patients with acute myeloid leukemia (AML) that revealed quantitative methylation patterns correlated with patient survival. Based on these results, a prognostic model was built which categorizes a patient's risk—either in a good or poor prognosis group. The findings provided herein support the use of genomic methylation markers for improved molecular classification and disease management in adult AML. Also, the results provide insight into the pathophysiology of AML and offer novel AML gene targets. Thus provided are methods and compositions for the prognosis of a subject suffering from acute myeloid leukemia (AML) based on the methylation state of nucleic acids. The methods may used alone to determine a patient's prognosis or in combination with other prognostic factors or markers such as gene expression.

### RELATED APPLICATIONS

This application is a national stage of international patent application number PCT/US2006/030256, filed on Aug. 2, 2006, which claims the benefit of U.S. Provisional Patent Application No. 60/705,068 filed Aug. 2, 2005 and U.S. Provisional Patent Application No. 60/705,069 filed Aug. 3, 2006, each entitled “Methods And Compositions For Disease Prognosis Based On Nucleic Acid Methylation,” naming Dirk van den Boom and Mathias Ehrich as inventors, and bearing attorney docket no. SEQ-4098-PV and SEQ-4098-PV2, respectively. Each of these patent applications is incorporated herein by reference in its entirety.

### FIELD OF THE INVENTION

The present invention relates to diagnostic and prognostic applications in the field of medicine and biotechnology. More specifically, the invention relates to methods and compositions for the prognosis of a subject suffering from acute myeloid leukemia (AML) based on the methylation state of nucleic acids alone or in combination with other prognostic markers such as gene expression.

### BACKGROUND

Genetic information is stored not only in the sequential arrangement of four nucleotide bases, but also in covalent modification of selected bases (see, e.g., Robertson et al., Nature Rev. Genet. 1:11-19 (2000)). One of these covalent modifications is methylation of cytosine nucleotides, particularly cytosines adjacent to guanine nucleotides in “CpG” dinucleotides. Covalent addition of methyl groups to cytosine within CpG dinucleotides is catalyzed by proteins from the DNA methyltransferase (DNMT) family (Amir et al., Nature Genet. 23:185-88 (1999); Okano et al., Cell 99:247-57 (1999)). In the human genome, CpG dinucleotides are generally under represented, and many of the CpG dinucleotides occur in distinct areas called CpG islands. A large proportion of these CpG islands can be found in promoter regions of genes. The conversion of cytosine to 5′-methylcytosine in promoter associated CpG islands has been linked to changes in chromatin structure and often results in transcriptional silencing of the associated gene. Transcriptional silencing by DNA methylation has been linked to mammalian development, imprinting and X-Chromosome inactivation, suppression of parasitic DNA and numerous cancer types (see, e.g., Li et al., Cell 69:915-26 (1992); Okano et al., Cell 99:247-57 (1999)). Detected changes in the methylation status of DNA can serve as markers in the early detection of neoplastic events (Costello et al., Nature Genet. 24:132-38 (2000)).

The interest in genomic methylation has fueled the development of several methods for assessment of cytosine methylation. Many of these techniques can only analyze a restricted set of CpG sites in their target regions and have to extrapolate the methylation status to the whole region (Cobra, MSP, restriction techniques, primer extension, PNA-MALDI TOF, Methylight and others). Issues with misinterpretation of the methylation status have been reported. Of particular importance are complications that arise for those methods restricted to selected CpGs specifically when their methylation within the examined genomic region is inconsistent. Other techniques assess several CpG sites at once by simultaneous hybridization of multiple oligonucleotides (e.g. Microarray, Primer extension) to amplification products of bisulfite treated DNA. Hybridization based techniques for methylation analyses are compromised by the effect of the bisulfite treatment. The degenerated nucleic acid code (reduction from four to mainly three bases) decreases the specificity of hybridization oligos. Due to the high density of CpG sites within CpG rich regions, the oligo length cannot be elongated arbitrarily without the incorporation of ambiguous bases (C/T).

Studies demonstrating the practical use of DNA methylation analysis in a clinical environment are scarce. This is due, at least in part, to the technical limitations facing DNA methylation research. A few DNA methylation analysis techniques have been used, but each method has its limitations. See, for example, U.S. Pat. No. 6,214,556 directed to methods for producing complex DNA methylation fingerprints. The methods of this patent amplify fragments of genomic DNA that have been treated with bisulfite using degenerated oligonucleotides or oligonucleotide that are complimentary to adaptor oligonucleotides that have been ligated to the fragmented genomic DNA. Methods such as these are prone to false positive results and are limited in accurate methylation assessment to a single cytosine position per analysis. Often times they require large amounts of high quality genomic DNA and are labor intensive.

Technical limitations have prevented large scale DNA methylation studies that would offer a powerful tool for the diagnosis and prognosis of a wide variety of diseases, including acute myeloid leukemia (AML). AML is a cancer of the bone marrow and blood characterized by the rapid uncontrolled growth of immature white blood cells known as myelocytes. The incidence of AML is approximately 3.6 per 100,000 people per year, and the age-adjusted incidence is higher in men than in women (4.4 versus 3.0). The disease is more common in adults than in children, with the average age at diagnosis being more than 65 years. A significant increase in AML incidence has occurred over the past ten years, and, although treatment of acute myeloid leukemia (AML) has improved dramatically over the past 30 years, the majority of patients with this disease will die within two years of diagnosis. Therefore, there is a need for earlier diagnosis, more accurate prognosis and improved, patient-specific therapeutic regimens to provide greater options for patients who suffer from AML. More specifically, there is a need for reliable, cost effective, high throughput DNA methylation analysis tools and methods to evaluate potential methylated sites, to associate methylation sites with AML, and to develop AML-related prognostic and pharmacogenomic methylation markers.

### SUMMARY

A large scale DNA methylation study was performed in patients with AML that revealed quantitative methylation patterns correlated with patient survival. Based on these results, a prognostic model was built which categorizes a patient's risk. The prognostic model can be utilized to determine a good or poor prognosis for a subject. The findings provided herein support the use of genomic methylation markers for improved molecular classification and disease management in adult AML. Also, the results provide insight into the pathophysiology of AML and offer novel AML gene targets.

The methods described herein have been practiced using a novel approach for DNA methylation analysis. This method employs MALDI-TOF analysis to overcome the limitations of previous large scale methylation analysis methods. Using a combination of four base specific cleavage reactions, each CpG of a target region can be analyzed individually and is represented by multiple indicative mass signals. The acquired information about the methylation status of the examined region is based on numerous independent observations. The redundancy of this information can be leveraged to achieve higher confidence in qualitative analysis, and to obtain highly accurate averages in quantitative analysis with small standard deviations. The present methods may be customized to meet individual needs in DNA methylation analysis. For example, discovery of methylation in large stretches of genomic DNA with a single cleavage reaction, methylation ratio analysis, where fractions of methylated DNA are as low as 5% may be detected in mixtures of methylated and non-methylated template, and methylation pattern analysis, where the methylation status of each CpG within a target region can be determined as a group or independently. The general applicability of these methods have been demonstrated by reconstructing the described methylation sites for IGF2/H19 using cloned DNA as well as genomic DNA (see Examples 1-7). The semi-quantitative assessment of methylation in larger target regions spanning multiple CpG sites was demonstrated and was able to accurately analyze methylation down to ratio's of approximately 5%. The large-scale analysis of methylation in AML is a first implementation of the method for quantitative assessment of methylation ratios in a high-throughput format to predict AML patient outcome.

Thus, provided herein are methods for determining an AML prognosis for a subject, comprising: a) determining the methylation state of (one or more) target gene regions in a nucleic acid from the subject; and b) comparing the methylation state of (a) to the methylation state of the target gene regions in nucleic acids from subjects having known AML outcomes; whereby the AML prognosis for the subject is determined from step (b). In some embodiments, the methylation states of the target gene regions in nucleic acids from subjects are determined before the methylation state of the (one or more) target regions in the nucleic acid from the subject is determined. In some embodiments, the methylation state in each of step (a) and (b) is characterized by comparing the ratio of a methylated nucleic acid base to an unmethylated nucleic acid base.

Some embodiments are directed to a method for predicting the prognosis of a subject who suffers from AML where the prognosis is correlated with the methylation state of a nucleic acid sample from the subject. In certain embodiments, the method comprises the steps of (a) determining in the nucleic acid sample the characteristic methylation state of a nucleic acid target gene region by identification of methylation sites of the nucleic acid target gene region; (b) determining in a nucleic acid sample from a subject or group of subjects having AML, the characteristic methylation state of the nucleic acid target gene region by identification of methylation sites of the nucleic acid target gene; and (c) comparing the characteristic methylation state of step a and of step b to determine the prognosis of the subject. In some embodiments, the method comprises (a) determining in the nucleic acid sample the characteristic methylation state of a nucleic acid target gene region by identification of methylation sites of the nucleic acid target gene region; (b) providing the characteristic methylation state of a subject or group of subjects having AML, the characteristic methylation state of the nucleic acid target gene region by identification of methylation sites of the nucleic acid target gene; and (c) comparing the characteristic methylation state of step (a) and of step (b) to determine the prognosis of the subject.

In a related embodiment, the characteristic methylation state in each of step (a) and (b) is characterized by comparing the ratio of a methylated nucleic acid base to an unmethylated nucleic acid base and where step (c) comprises comparing the ratio in step (a) to the ratio in step (b).

In some embodiments, the number of target gene regions is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 105, 110, 115, 120, 125, 130, 135, 140, 145, 147, 150 or more.

In certain embodiments, the comparison of methylation states or characteristic methylation states is made by use of a classification algorithm.

In particular embodiments, the reagent that modifies unmethylated cytosine to produce uracil is bisulfite. In certain embodiments, the methylated or unmethylated nucleic acid base is cytosine. In another embodiment, a non-bisulfite reagent modifies unmethylated cytosine to produce uracil.

In some embodiments, the prognosis is the probability of surviving the leukemia for a certain period of time, the probability of AML relapse after induction therapy, or the probability of a complete remission.

In selected embodiments, the methods for determining the methylation state of (one or more) target gene regions may include treating a target nucleic acid molecule with a reagent that modifies nucleotides of the target nucleic acid molecule as a function of the methylation state of the target nucleic acid molecule, amplifying treated target nucleic acid molecule, fragmenting amplified target nucleic acid molecule, and detecting one or more amplified target nucleic acid molecule fragments, and based upon the fragments, such as size and/or number thereof, identifying the methylation state of a target nucleic acid molecule, or a nucleotide locus in the nucleic acid molecule, or identifying the nucleic acid molecule or a nucleotide locus therein as methylated or unmethylated.

Fragmentation can be performed, for example, by treating amplified products under base specific cleavage conditions. Detection of the fragments can be effected by measuring or detecting a mass of one or more amplified target nucleic acid molecule fragments, for example, by mass spectrometry such as MALDI-TOF mass spectrometry. Detection also can be affected, for example, by comparing the measured mass of one or more target nucleic acid molecule fragments to the measured mass of one or more reference nucleic acid, such as measured mass for fragments of untreated nucleic acid molecules. In an exemplary method, the reagent modifies unmethylated nucleotides, and following modification, the resulting modified target is specifically amplified.

In some embodiments, the methods for determining the methylation state of (one or more) target gene regions may include treating a target nucleic acid molecule with a reagent that modifies a selected nucleotide as a function of the methylation state of the selected nucleotide to produce a different nucleotide; contacting the treated target nucleic acid molecule with a primer containing one or more nucleotides complementary to the selected nucleotide, or one or more nucleotides complementary to the different nucleotide; treating the contacted target nucleic acid molecule under nucleic acid synthesis conditions, whereby nucleotides are synthesized onto primers hybridized to the target nucleic acid molecule; treating the synthesized products under base specific cleavage conditions; and detecting the products of the cleavage treatment, where a target nucleic acid molecule containing one or more methylated or unmethylated selected nucleotides is determined according to the number of cleavage products or according to a comparison between one or more cleavage products and one or more references.

In certain embodiments, the methods for determining the methylation state of (one or more) target gene regions may include treating a target nucleic acid molecule with a reagent that modifies a selected nucleotide as a function of the methylation state of the selected nucleotide to produce a different nucleotide; amplifying the treated target nucleic acid molecule to form an amplification product; contacting the treated target nucleic acid molecule with a primer containing one or more nucleotides complementary to a nucleotide complementary to the selected nucleotide, or one or more nucleotides complementary to a nucleotide complementary to the different nucleotide; treating the contacted target nucleic acid molecule under nucleic acid synthesis conditions, whereby nucleotides are synthesized onto primers hybridized to the target nucleic acid molecule; treating the synthesized products under base specific cleavage conditions; and detecting the products of the cleavage treatment, where a target nucleic acid molecule containing one or more methylated or unmethylated selected nucleotides is determined according to the number of cleavage products or according to a comparison between one or more cleavage products and one or more references.

In some embodiments, the methods for determining the methylation state of (one or more) target gene regions may include treating a target nucleic acid molecule with a reagent selected from among a reagent that modifies an unmethylated selected nucleotide to produce a different nucleotide, and a reagent that modifies a methylated selected nucleotide to produce a different nucleotide; specifically amplifying the treated target nucleic acid molecule by a method selected from: (i) contacting the treated target nucleic acid molecule with a primer that specifically hybridizes to a target nucleic acid region containing one or more of the selected nucleotides or one or more of the different nucleotides, and treating the contacted target nucleic acid molecule under nucleic acid synthesis conditions, and (ii) amplifying the treated target nucleic acid molecule to form an amplification product, contacting the amplification product with a primer that specifically hybridizes to a target nucleic acid region containing one or more of the selected nucleotides, or one or more of the different nucleotides, and treating the contacted amplification product under nucleic acid synthesis conditions; treating the amplified products with base specific cleavage conditions; and detecting the products of the cleavage treatment, where a target nucleic acid molecule containing one or more methylated or unmethylated selected nucleotides is indicated by an observation selected from among: the presence of two or more cleavage products, the presence of only a single cleavage product, the presence of one or more cleavage products greater than the number of reference nucleic acid molecules, the presence of one or more cleavage products fewer than the number of reference nucleic acid molecules, the presence of the same number of cleavage products as reference nucleic acid molecules, a change in the mass of one or more cleavage products compared to a reference nucleic acid molecule mass, and one or more cleavage products that are the same mass as a reference nucleic acid molecule mass.

In certain embodiments, the methods for determining the methylation state of (one or more) target gene regions may include treating a target nucleic acid molecule with a reagent that modifies unmethylated cytosine to produce uracil; specifically amplifying the treated target nucleic acid molecule with a primer that contains one or more guanine nucleotides; base specifically cleaving the amplified products; and detecting the cleaved products, where the presence of two or more fragments indicates that the target nucleic acid molecule contains one or more methylated cytosines. Another example includes a method of identifying an unmethylated nucleic acid molecule, by treating a target nucleic acid molecule with a reagent that modifies unmethylated cytosine to produce uracil; specifically amplifying the treated target nucleic acid molecule with a primer that contains one or more adenine nucleotides; base specifically cleaving the amplified products; and detecting the cleaved products, where the presence of two or more fragments indicates that the target nucleic acid molecule contains one or more unmethylated cytosines.

In some embodiments, the methods for determining the methylation state of (one or more) target gene regions may include treating a target nucleic acid molecule with a reagent that modifies unmethylated cytosine to produce uracil; specifically amplifying the treated target nucleic acid molecule with a primer that contains one or more guanine nucleotides; base specifically cleaving the amplified products; and detecting the mass of the cleaved products, where: a change in mass of one or more cleaved products compared to a reference mass indicates that a nucleotide locus in a target is methylated. A similar exemplary method includes a method for identifying the nucleotide locus of an unmethylated nucleotide in a nucleic acid, by treating a target nucleic acid molecule with a reagent that modifies unmethylated cytosine to produce uracil; specifically amplifying the treated target nucleic acid molecule with a primer that contains one or more adenine nucleotides; base specifically cleaving the amplified products; and detecting the mass of the cleaved products, where: a change in mass of one or more cleaved products compared to a reference mass indicates that a nucleotide locus in a target is methylated.

In certain embodiments, the methods for determining the methylation state of (one or more) target gene regions may include treating a target nucleic acid molecule to deaminate unmethylated cytosine nucleotides; specifically amplifying the treated target nucleic acid molecule with a primer that specifically hybridizes to a pre-determined first region in the target nucleic acid molecule containing one or more cytosine nucleotides; base specifically cleaving the amplified products; and detecting the mass of the cleaved products, where: a change in mass of one or more cleaved products compared to a reference mass indicates that a nucleotide locus in a second region in a target is methylated, where the first region and second region do not overlap.

In some embodiments, the methods for determining the methylation state of (one or more) target gene regions may include treating a target nucleic acid molecule with a reagent that modifies unmethylated cytosine to produce uracil; specifically amplifying the treated target nucleic acid molecule with a primer that contains one or more guanine nucleotides; base specifically cleaving the amplified products; and cleaving or simulating cleavage of a reference nucleic acid with the same cleavage reagent(s); detecting the mass of the cleaved products; determining differences in the mass signals between the target nucleic acid molecule fragments and the reference fragments; and determining a reduced set of sequence variation candidates from the differences in the mass signals and thereby determining sequence variations in the target compared to the reference nucleic acid, where methylation of a nucleotide locus is indicated by the nucleotide locus of a sequence variation. In another example of the methods, combinations and kits provided herein, a method, combination and kit is provided for identifying the nucleotide locus of a methylated nucleotide in a nucleic acid, by treating a target nucleic acid molecule with a reagent that modifies unmethylated cytosine to produce uracil; amplifying the treated target nucleic acid molecule to form a first amplification product; specifically amplifying the first amplification product with a primer that contains one or more cytosine nucleotides to form a second amplification product; base specifically cleaving the second amplification products; cleaving or simulating cleavage of a reference nucleic acid with the same cleavage reagent(s); detecting the mass of the cleaved products; determining differences in the mass signals between the target nucleic acid molecule fragments and the reference fragments; and determining a reduced set of sequence variation candidates from the differences in the mass signals and thereby determining sequence variations in the target compared to the reference nucleic acid, where methylation of a nucleotide locus is indicated by the nucleotide locus of a sequence variation.

In certain embodiments, the methods for determining the methylation state of (one or more) target gene regions may include treating two or more different target nucleic acid molecules with a reagent that modifies a selected nucleotide as a function of the methylation state of the selected nucleotide to produce a different nucleotide; contacting the treated target nucleic acid molecules with a primer containing one or more nucleotides complementary to the selected nucleotide, or one or more nucleotides complementary to the different nucleotide; treating the contacted target nucleic acid molecules under nucleic acid synthesis conditions, whereby nucleotides are synthesized onto primers hybridized to the target nucleic acid molecules; treating the synthesized products under base specific cleavage conditions; and detecting the products of the cleavage treatment, where target nucleic acid molecules containing one or more methylated or unmethylated selected nucleotides are determined according to a comparison between one or more cleavage products and one or more references.

In some embodiments, the methods for determining the methylation state of (one or more) target gene regions may include treating a target nucleic acid molecule with a reagent that modifies a selected nucleotide as a function of the methylation state of the selected nucleotide to produce a different nucleotide; contacting the treated target nucleic acid molecule with a primer containing one or more nucleotides complementary to the selected nucleotide, or one or more nucleotides complementary to the different nucleotide; treating the contacted target nucleic acid molecule under nucleic acid synthesis conditions, whereby nucleotides are synthesized onto primers hybridized to the target nucleic acid molecules; treating the synthesized products under fragmentation conditions; and detecting the products of the fragmentation treatment by mass spectrometry, where target nucleic acid molecules containing one or more methylated or unmethylated selected nucleotides are determined according to the number of fragmentation products or according to a comparison between one or more fragmentation products and one or more references. Similarly, methods are provided for identifying one or more methylated or unmethylated nucleotides in a nucleic acid, by treating a target nucleic acid molecule with a reagent that modifies a selected nucleotide as a function of the methylation state of the selected nucleotide to produce a different nucleotide; contacting the treated target nucleic acid molecule with a blocking oligonucleotide containing one or more nucleotides complementary to the selected nucleotide, or one or more nucleotides complementary to the different nucleotide; treating the contacted target nucleic acid molecule under nucleic acid synthesis conditions, where nucleotide synthesis is inhibited when the blocking oligonucleotide is hybridized to a target nucleic acid molecule; treating the synthesized products under base specific cleavage conditions; and detecting the products of the cleavage treatment, where a target nucleic acid molecule containing one or more methylated or unmethylated selected nucleotides are determined according to the number of cleavage products or according to a comparison between one or more cleavage products and one or more references.

In certain embodiments, the methods for determining the methylation state of (one or more) target gene regions may include treating a target nucleic acid molecule with a reagent that modifies a selected nucleotide as a function of the methylation state of the selected nucleotide to produce a different nucleotide; contacting the target nucleic acid molecule with a cleavage reagent that selectively cleaves the target nucleic acid at a site containing one or more methylated selected nucleotides or one or more unmethylated selected nucleotides, or with a cleavage reagent that selectively cleaves the treated target nucleic acid at a site containing one or more selected nucleotides or one or more different nucleotides; treating the contacted target nucleic acid molecule under nucleic acid synthesis conditions, where a target nucleic acid molecule not cleaved is amplified; treating the amplified products under base specific cleavage conditions; and detecting the products of the cleavage treatment, where a target nucleic acid molecule containing one or more methylated or unmethylated selected nucleotides are determined according to the number of cleavage products or according to a comparison between one or more cleavage products and one or more references.

In some embodiments, the methods for determining the methylation state of (one or more) target gene regions may include contacting the target nucleic acid molecule with a primer and treating the contacted target nucleic acid molecule under nucleic acid synthesis conditions, where a strand complementary to the target nucleic acid molecule is synthesized; contacting the target nucleic acid-synthesized product duplex with a methyltransferase reagent whereby methylation in a CpG sequence of the target nucleic acid also is present in the complementary CpG sequence of the synthesized product; repeating the primer and methyltransferase reagent contacting steps to form a second synthesized product having the same sequence of nucleotides and methylation state of CpG nucleotides as present in the target nucleic acid molecule; treating synthesized products with a reagent that modifies a selected nucleotide as a function of the methylation state of the selected nucleotide to produce a different nucleotide; treating the reagent-treated products under base specific cleavage conditions; and detecting the products of the cleavage treatment, where a target nucleic acid molecule containing one or more methylated or unmethylated selected nucleotides are determined according to the number of cleavage products or according to a comparison between one or more cleavage products and one or more references.

In certain embodiments, the methods for determining the methylation state of (one or more) target gene regions may include identifying one or more methylated or unmethylated nucleotides in a nucleic acid, where the amplified products are cleaved by base specific cleavage conditions selected from chemical conditions, physical conditions, enzymatic base specific cleavage conditions, and combinations thereof. For example, the amplified products can be cleaved by an RNase, a DNase, an alkaline compound, piperidine formate, piperidine, dimethyl sulfate, hydrazine, sodium chloride, and combinations thereof.

In some embodiments, the methods for determining the methylation state of (one or more) target gene regions may include identifying one or more methylated or unmethylated nucleotides in a nucleic acid, where the amplifying step includes transcription. In such methods, the nucleoside triphosphates incorporated into the transcript can include three rNTPs and one dNTP. For example, the one dNTP can be selected from dCTP, dTTP, dATP and dGTP. In another example, the one dNTP can be selected from dCTP and dTTP, and the transcript can be cleaved by RNase A.

In certain embodiments, the methods for determining the methylation state of (one or more) target gene regions may include identifying one or more methylated or unmethylated nucleotides in a nucleic acid, where the intensity of one or more sample measured masses is compared to the intensity of one or more reference masses. Similarly, also provided herein are methods of identifying one or more methylated or unmethylated nucleotides in a nucleic acid, where two or more nucleic acid samples are pooled, and the intensity of one or more sample measured masses is compared to the intensity of one or more reference masses. In such methods an incompletely converted target nucleic acid molecule can be distinguished from a methylated target nucleic acid molecule.

In some embodiments, the methods for determining the methylation state of (one or more) target gene regions may be used for distinguishing between a false positive methylation specific amplification and a true methylation specific amplification, by, for example, treating a target nucleic acid molecule with a reagent that modifies an unmethylated selected nucleotide to produce a different nucleotide; contacting the treated target nucleic acid molecule with a methylation state specific primer complementary to a first target nucleic acid region containing one or more of the selected nucleotides; treating the contacted target nucleic acid molecule under nucleic acid synthesis conditions; treating the synthesized products under base specific cleavage conditions; and detecting the mass of the cleaved products, where: a change in mass of one or more cleaved products compared to a reference mass indicates that a nucleotide locus in a second region in a target is methylated, where the second region does not overlap with the first region, whereby presence of one or more methylated loci in the second region confirms true methylation specific amplification.

In certain embodiments, the methods for determining the methylation state of (one or more) target gene regions may be used for identifying methylated nucleotides and thereby identify methylation patterns, which can be correlated with a disease, disease outcome, or outcome of a treatment regimen, by, for example, identifying methylated or unmethylated nucleotides, in accordance with the method of any of methods provided herein, in one or more nucleic acid molecules from one or more samples collected from one or more subjects having a known disease, disease outcome, or outcome of a treatment regimen; identifying methylated or unmethylated nucleotides, in accordance with the method of any of methods provided herein, in one or more nucleic acid molecules from one or more samples collected from one or more normal subjects; and identifying the differently methylated or unmethylated nucleotides between the one or more nucleic acid molecules of step (a) and the one or more nucleic acid molecules of step (b); whereby the differently methylated or unmethylated nucleotides identify methylation correlated with a disease, disease outcome, or outcome of a treatment regimen.

In some embodiments, the methods for determining the methylation state of (one or more) target gene regions may be used for diagnosing a disease, deciding upon a treatment regimen, or determining a disease outcome in a subject, by, for example, identifying one or more methylated or unmethylated nucleotides in one or more nucleic acid molecules from one or more samples collected from a subject; and comparing the methylated or unmethylated nucleotides in the one or more nucleic acid molecules with one or more reference nucleic acid molecules correlated with a known disease, disease outcome, or outcome of a treatment regimen; whereby methylated or unmethylated nucleotides that are the same as the reference nucleic acid molecules identify the disease, disease outcome, or outcome of a treatment regimen in the subject. The methods, combinations and kits provided herein also can be used in deciding upon a treatment regimen, or determining a disease outcome in a subject, by, for example, identifying one or more methylated or unmethylated nucleotides in one or more nucleic acid molecules from one or more samples collected from a subject; and comparing the methylated or unmethylated nucleotides in the one or more nucleic acid molecules with one or more reference nucleic acid molecules correlated with a known disease, disease outcome, or outcome of a treatment regimen; whereby methylated or unmethylated nucleotides that are different from the reference nucleic acid molecules identify the disease, disease outcome, or outcome of a treatment regimen in the subject.

In certain embodiments, the methods for determining the methylation state of (one or more) target gene regions may be used in determining a methylation state at one or more nucleotide loci correlated with an allele, by, for example, pooling nucleic acid molecules containing a known allele; identifying one or more methylated or unmethylated nucleotide loci in the nucleic acid molecules containing the known allele; identifying the methylation state of the corresponding nucleotide loci in nucleic acid molecules that do not contain the allele; and comparing the methylation state of the nucleotide loci in allele-containing nucleic acid molecules to the methylation state of nucleotide loci in allele-lacking nucleic acid molecules, whereby differences in methylation state frequency at one or more loci identify the different loci as correlated with the allele. Similarly, the methods, combinations and kits provided herein can be used for determining an allele correlated with a methylation state at one or more nucleotide loci, by forming a first pool of nucleic acid molecules containing one or more known methylated or unmethylated nucleotide loci, which loci were identified in accordance with the methods provided herein; identifying the frequency at which one or more alleles are present in the pooled nucleic acid samples; identifying the allele frequency at which one or more alleles are present in a second pool of nucleic acid molecules having nucleotide loci with different methylation state relative to the first pooled nucleic acid molecules; and comparing the allelic frequency in the first pool of nucleic acid molecules to the allelic frequency in the second pool of nucleic acid molecules, whereby differences in allelic frequency identify the one or more loci as correlated with the allele.

In some embodiments, the methods for determining the methylation state of (one or more) target gene regions may be used for determining the probable identity of one or more alleles, by, for example, identifying one or more methylated or unmethylated nucleotides a nucleic acid molecule; and determining the frequency of presence of one or more alleles with the presence of one or more methylated or unmethylated nucleotides where the probable identity of the allele is determined.

Also provided herein are combinations and kits for determining the methylation state of a target nucleic acid molecule. Kits can include a reagent that modifies one or more nucleotides of the target nucleic acid molecule as a function of the methylation state of the target nucleic acid molecule, one or more methylation specific primers capable of specifically hybridizing to a treated target nucleic acid molecule, and one or more compounds capable of fragmenting an amplified target nucleic acid molecule. The one or more compounds capable of fragmenting amplified nucleic acid products can include an RNase, a DNase, an alkaline compound, piperidine formate, piperidine, dimethyl sulfate, hydrazine, sodium chloride, and combinations thereof. For example, kits provided herein can include one or more RNases

In some embodiments, the methylation state is determined by mass spectrometry.

In some embodiments, the methylation state is determined by multiplexed hME assays, fluorescence-based real-time PCR, methylation-sensitive single nucleotide primer extension, methylated CpG island amplification, methylation-specific PCR, restriction landmark genomic scanning, methylation-sensitive-representational difference analysis (MS-RDA), methylation-specific AP-PCR (MS-AP-PCR) methyl-CpG binding domain column/segregation of partly melted molecules (MBD/SPM), or bisulphite sequencing direct. Specific methods for determining the methylation state may include combined bisulfite restriction analysis (COBRA), PyroMeth or MethyLight.

In some embodiments, the AML prognosis for the subject determined in step (b) or step (c) in the preceding embodiments is combined with an AML-related prognostic factor based on known morphology, cytochemistry, immunophenotype, cytogenetics or molecular techniques to provide a more predictive prognosis for the subject. In a related embodiment, the AML-related molecular technique is a gene expression profile. In a further related embodiment, the gene expression profile consists of one or more target gene regions and/or genes regulated by one or more target gene regions.

In some embodiments, the method for predicting the prognosis of a subject who suffers from AML further comprises administering an AML treatment based upon the AML prognosis. In a related embodiment, the AML treatment is a good prognosis treatment regimen or a poor prognosis treatment regimen. In a further related embodiment, the AML treatment is selected from the group consisting of administering a non-standard, non-aggressive or experimental chemotherapy agent chemotherapy agent, performing an allogeneic stem cell transplant, administering all-trans-retinoic acid, administering a novel therapy, administering palliative care, and combinations of the foregoing. A “novel therapy” as used herein refers to an investigational treatment (e.g., monoclonal antibodies, new consolidation chemotherapy regimens, multiple drug resistance inhibitors, biological modifier therapies, and demethylating agents). In another related embodiment, the AML treatment is a standard AML treatment course. Standard AML treatment includes a 7-day continuous infusion of cytarabine, and a 3-day course of an anthracycline. The anthracyclines include daunorubicin (Cerubidine), doxorubicin (Adriamycin, Rubex), epirubicin (Ellence, Pharmorubicin), and idarubicin (Idamycin). The standard treatment is often supplemented by performing a blood transfusion, performing a platelet transfusion, administering antibiotics and blood cell growth factors.

In certain embodiments, the methods described herein may be utilized to detect the presence or absence of a disease in a tissue or cell that correlates with changes in the methylation state of the tissue or cell, or classify the susceptibility of a tissue or cell to a disease where the disease is correlated with changes in the methylation state of the tissue or cell. In another embodiment, the methods described herein may be utilized for the early detection AML before AML is otherwise detectable by current diagnostic methods known in the art. For example, the methods described herein may be utilized to detect an altered methylation state associated with the presence of AML before physical indicators manifest (e.g., decreased leukocyte counts).

In a related embodiment, the disease state is a hematologic cancer. The hematologic cancer sometimes is a blood myeloid leukemia, acute myeloid leukemia (AML), chronic myeloid leukemia (CML), acute lymphoblastic leukemia (ALL), chronic lymphocytic leukemia (CLL), blood myeloproliferative diseases, blood multiple myeloma, blood myelodysplasic syndrome, Hodgkin's disease and non-Hodgkin's lymphoma. The hematologic cancer often is acute myeloid leukemia.

In some embodiments, the nucleic acid target gene is one or more of ABO1, ABCB1, ACTG1, ADFP, AFP, AGT, AMIGO2, ANGPT1, APOB, APOC1, AQP1, ARHGAP22, ATP8B4, AZGP1, BAALC, BAI2, BCL11A, C10orf38, CD3D, CDC42EP4, CDH5, CDKN2A, CDKN2A, CDX2, CEACAM6, CEBPA, CKMT1, CNN3, COL1A1, CTNNAL1, D2S448, DAPK1, DLK1, DMPK, DPEP2, DUSP4, E.cad (CDH1), EDG1, EML4, EMR1, ERalpha, ESR1, ETS1, EVI1, FARP1, FGFR1, FHL2, FLI1, FLJ21820, FLJ23058, FLJ25409, FLT3, FN14, FOXO1A, GAGED2, GAS7, GLUL, GNG2, GS3955, GSTP1, GUCY1A3, GYPC, HIP1, HOXA1, HOXA10, HOXA10, HOXA11, HOXA3, HOXA4, HOXA7, HOXA9, HOXA9, HOXB2, HOXB2, HOXB5, HOXD11, HOXD13, ID3, IFI27, IL6ST, ISG20, KIAA0476, KIAA0830, KIAA1447, KRT13, LAD1, LAMB3, LCN2, LGMN, LOC114990, LOC55971, LOC57228, LRP6, MAGEA3, MAP7, MEIS1, MGC14376, MGC16121, MGMT, MGP, MIG2, MSLN, MYOD, N33, NBL1, NFIB, NFKB1, NFKBIB, Notch4, NR2F2, NRP1, p16, p53, PAGE5, PBX3, PHEMX, PIK3R4, PITX2, PLCG1, PLEKHC1, PMP22, PRAME, PRG2, PRO2730, PSCB5, PVALB, RARB, RASSF1, RBP1, RGS16, RIS1, RPL22, RUNX3, S100P, SATalpha, SCAP2, SDK2, SDS-RS1, SELENBP1, SEMA3F, SERPINA3, SERPINA5, SERPINB5, SFTPB, SLC2A1, SLC6A8, SLC7A5, SLC7A7, SMG1, SNX9, SOCS1, SPI1, SPUVE, STGB3A1, STX1A, TACSTD2, TBXAS1, TCF4, TGM2, TM4SF2, TMEPAI, TNA, TNFRSF12A, TRIB2, TUBB, TUBB5, TUCAN, UGCG, UGCGL2, URB, VIL2 or ZD52F10. More specifically, the nucleic target gene region is one or more of chr7:27116632-27117064, chr7:87067801-87068530, chr17:77042426-77043830, chr17:77080311-77081236, chr17:77092731-77097121, chr17:77100095-77101608, chr17:77069230-77070518, chr17:77109501-77110986, chr17:77042426-77043830, chr17:77029988-77030478, chr9:19116981-19118080, chr4:74590458-74591581, chr1:227812884-227813798, chr12:45759345-45760487, chr8:109050870-109052632, chr2:21241007-21241697, chr19:50103362-50104640, chr7:30917877-30918305, chr10:49482759-49483458, chr15:48261515-48262578, chr7:99432944-99433641, chr8:104221803-104222666, chr1:31730622-31732925, chr2:60634325-60635988, chr10:15294961-15295393, chr11:117735176-117735778, chr17:68818372-68820477, chr16:64970452-64970801, chr9:21964481-21965407, chr9:21984002-21986010, chr13:27438257-27441645, chr19:46951004-46951263, chr19:38483802-38486884, chr15:41673107-41674117, chr1:95164227-95165904, chr17:45633888-45634168, chr9:110814664-110815955, chr2:1630803-1632607, chr9:89302236-89303737, chr14:100262505-100263352, chr19:50962440-50967107, chr16:66584476-66584997, chr8:29261385-29265966, chr16:67328436-67329945, chr1:101474835-101475533, chr2:42370638-42371668, chr19:6773069-6773804, chr6:152170416-152171564, chr6:152220837-152221985, chr11:127896823-127897921, chr3:170346622-170347240, chr13:97592201-97594442, chr8:38444050-38445731, chr2:105381112-105382516, chr11:128067782-128070321, chr2:20885847-20886615, chr17:77044897-77045932, chr8:38362799-38363952, chr13:27572029-27573370, chr16:3009898-3011506, chr13:40136302-40139950, chrX:52562488-52562915, chr17:10041560-10043365, chr1:180626122-180628386, chr14:51396700-51504379, chr2:12878166-12880958, chr11:67107562-67107961, chr4:156807549-156808942, chr2:127129968-127130841, chr7:75012514-75013929, chr7:27109607-27110104, chr7:27178842-27181021, chr7:26954490-26956868, chr7:27191476-27192254, chr7:27116456-27117043, chr7:27135998-27137263, chr7:27162027-27163192, chr7:27170341-27173087, chr7:27171485-27172005, chr17:43975168-43976572, chr17:47094807-47096211, chr17:44025323-44026657, chr2:176797014-176798012, chr2:176782362-176783986, chr1:23758071-23758999, chr14:92536844-92537906, chr5:55306022-55307474, chr15:86965182-86966156, chr1:152184798-152186158, chr11:94510552-94511964, chr17:77042327-77043930, chr17:36937236-36938325, chr1:198656619-198657489, chr1:206927634-206929017, chr9:129929273-129931165, chr14:92329862-92330877, chr16:4421737-4422733, chr7:97867835-97868558, chr12:499-49473-49950878, chr12:12310747-12312008, chrX:151617792-151618218, chr6:136851198-136852915, chr2:66514547-66516842, chr17:1565756-1567012, chrX:133405569-133406409, chr10:131194455-131195415, chr12:14887647-14888003, chr14:52486660-52488289, chr16:737974-738711, chr11:17697266-17700455, chr8:15441963-15442472, chr1:19842644-19844710, chr9:14302754-14305551, chr4:103640925-103642461, chr4:103641494-103642135, chr6:3378321-3378733, chr15:94674214-94678925, chr10:33626928-33630403, chr9:21964379-21965506, chr17:7532020-7532764, <chrX:55263140-55263638, chr9:127547944-127550691, chr11:2249028-2249621, chr3:131947967-131948587, chr4:111761312-111764113, chr20:39198445-39200446, chr14:52486659-52488289, chr12:131502182-131503829, chr22:21231288-21231721, chr11:56950718-56951426, chr3:52286911-52288297, chr19:37763517-37764848, chr22:34695834-34697316, chr3:25444558-25614624, chr17:2521246-2521267, chr3:140740640-140741618, chr1:180839800-180840583, chr3:452-41283-452-43243, chr1:6182401-6182644, chr1:25127915-25131792, chr4:6726394-6727508, chr1:121061571-121062477, chr7:26870196-26871478, chr17:68943183-68943544, chr12:112207972-112209000, chr1:149612226-149612784, chr3:50166777-50168334, chr14:94147980-94160642, chr14:94147980-94160642, chr14:93710152-93710641, chr2:85954841-85956938, chr1:43195856-43197555, chrX:152591630-152592938, chr16:86459756-86461161, chr14:22361402-22361999, chr16:18844333-18845827, chr6:158163391-158165223, chr16:11255843-11258504, chr11:47356165-47356782, chr11:86188634-86189737, chr5:179951301-179951693, chr7:72545287-72546501, chr2:474-49875-47450330, chr7:138884485-138885973, chr18:51595863-51597029, chr20:36226841-36227186, chrX:37451260-37452579, chr20:56969114-56971016, chr3:450-42634-450-42968, chr16:3009897-3011506, chr2:12807024-12809817, chr6:3102201-3103617, chr19:6452891-6453611, chr19:53466622-53467353, chr9:113698241-113699794, chr13:95503032-95504110, chr3:113805901-113842867, chr6:159158871-159160963, or chr19:40715824-40716843.

In some embodiments the nucleic acid target gene is one or more of ABO1, ABCB1, ACTG1, ACTG1.01, ACTG1.01, ACTG1.02, ACTG1.02, ACTG1.03, ACTG1.06, ACTG1.09, APOC1, AZGP1, BAALC, BCL11A, C10orf38, CD3D, CDC42EP4, CDKN2A, CDKN2A, CEBPA, CKMT1, CNN3, CTNNAL1, D2S448, DAPK1, DLK1, DPEP2, DUSP4, E.cad (CDH1), EDG1, EMR1, ERalpha, ESR1, EVI1, FARP1, FGFR1, FHL2, FLI1, FLJ21820, FLJ23058, FLT3, FN14, FOXO1A, GAS7, GLUL, GNG2, GSTP1, GUCY1A3, GYPC, HOXA1, HOXA10, HOXA10, HOXA11, HOXA3, HOXA4, HOXA7, HOXA9, HOXA9, HOXB2, HOXB2, HOXB5, HOXD11, HOXD13, ID3, ISG20, KIAA0476, KIAA1447, KRT13, LCN2, LGMN, LOC55971, LRP6, MEIS1, MGC14376, MIG2, MSLN, MYOD, NBL1, NFKB1, Notch4, NR2F2, p16, p53, PBX3, PIK3R4, PITX2, PLCG1, PLEKHC1, PRG2, PRO2730, PSCB5, RARB, RASSF1, RBP1, RGS16, RIS1, RPL22, RUNX3, S100P, SATalpha, SCAP2, SEMA3F, SERPINA5, SLC2A1, SLC6A8, SMG1, SNX9, SOCS1, SPUVE, TACSTD2, TNFRSF12A, TUBB, TUCAN, UGCG, UGCGL2, VIL2 or ZD52F10. More specifically, the nucleic target gene region is one or more of chr7:27116632-27117064, chr7:87067801-87068530, chr17:77042426-77043830, chr17:77080311-77081236, chr17:77092731-77097121, chr17:77100095-77101608, chr17:77069230-77070518, chr17:77109501-77110986, chr17:77042426-77043830, chr17:77029988-77030478, chr19:50103362-50104640, chr7:99432944-99433641, chr8:104221803-104222666, chr2:60634325-60635988, chr10:15294961-15295393, chr11:117735176-117735778, chr17:68818372-68820477, chr9:21964481-21965407, chr9:21984002-21986010, chr19:38483802-38486884, chr15:41673107-41674117, chr1:95164227-95165904, chr9:110814664-110815955, chr2:1630803-1632607, chr9:89302236-89303737, chr14:100262505-100263352, chr16:66584476-66584997, chr8:29261385-29265966, chr16:67328436-67329945, chr1:101474835-101475533, chr19:6773069-6773804, chr6:152170416-152171564, chr6:152220837-152221985, chr3:170346622-170347240, chr13:97592201-97594442, chr8:38444050-38445731, chr2:105381112-105382516, chr11:128067782-128070321, chr2:20885847-20886615, chr17:77044897-77045932, chr13:27572029-27573370, chr16:3009898-3011506, chr13:40136302-40139950, chr17:10041560-10043365, chr1:180626122-180628386, chr14:51396700-51504379, chr11:67107562-67107961, chr4:156807549-156808942, chr2:127129968-127130841, chr7:27109607-27110104, chr7:27178842-27181021, chr7:26954490-26956868, chr7:27191476-27192254, chr7:27116456-27117043, chr7:27135998-27137263, chr7:27162027-27163192, chr7:27170341-27173087, chr7:27171485-27172005, chr17:43975168-43976572, chr17:47094807-47096211, chr17:44025323-44026657, chr2:176797014-176798012, chr2:176782362-176783986, chr1:23758071-23758999, chr15:86965182-86966156, chr1:152184798-152186158, chr17:77042327-77043930, chr17:36937236-36938325, chr9:129929273-129931165, chr14:92329862-92330877, chr7:97867835-97868558, chr12:12310747-12312008, chr2:66514547-66516842, chr17:1565756-1567012, chr14:52486660-52488289, chr16:737974-738711, chr11:17697266-17700455, chr1:19842644-19844710, chr4:103640925-103642461, chr6:3378321-3378733, chr15:94674214-94678925, chr9:21964379-21965506, chr17:7532020-7532764, chr9:127547944-127550691, chr3:131947967-131948587, chr4:111761312-111764113, chr20:39198445-39200446, chr14:52486659-52488289, chr11:56950718-56951426, chr3:52286911-52288297, chr19:37763517-37764848, chr3:25444558-25614624, chr17:2521246-2521267, chr3:140740640-140741618, chr1:180839800-180840583, chr3:452-41283-452-43243, chr1:6182401-6182644, chr1:25127915-25131792, chr4:6726394-6727508, chr1:121061571-121062477, chr7:26870196-26871478, chr3:50166777-50168334, chr14:94147980-94160642, chr1:43195856-43197555, chrX:152591630-152592938, chr16:18844333-18845827, chr6:158163391-158165223, chr16:11255843-11258504, chr11:86188634-86189737, chr2:474-49875-47450330, chr16:3009897-3011506, chr6:3102201-3103617, chr19:53466622-53467353, chr9:113698241-113699794, chr13:95503032-95504110, chr6:159158871-159160963 or chr19:40715824-40716843.

In certain embodiments, the nucleic acid target gene region is one or more of ACTG1, ACTG1.01, ACTG1.01, ACTG1.03, ACTG1.06, CKMT1, CNN3, DLK1, DUSP4, E.cad (CDH1), EVI1, FARP1, FGFR1, FHL2, FLJ23058, HOXA1, KIAA1447, MSLN, MYOD, NFKB1, PITX2, PLCG1, RBP1, RUNX3, TACSTD2 or ZD52F10. More specifically, the nucleic target gene region is one or more of chr17:77042426-77043830, chr17:77080311-77081236, chr17:77092731-77097121, chr17:77109501-77110986, chr17:77042426-77043830, chr15:41673107-41674117, chr1:95164227-95165904, chr14:100262505-100263352, chr8:29261385-29265966, chr16:67328436-67329945, chr3:170346622-170347240, chr13:97592201-97594442, chr8:38444050-38445731, chr2:105381112-105382516, chr17:77044897-77045932, chr7:27109607-27110104, chr17:77042327-77043930, chr16:737974-738711, chr11:17697266-17700455, chr4:103640925-103642461, chr4:111761312-111764113, chr20:39198445-39200446, chr3:140740640-140741618, chr1:25127915-25131792, chr2:474-49875-47450330, or chr19:40715824-40716843.

In certain embodiments, the nucleic acid target gene region is one or more of KIAA1447, ZD52F10, HOXA1, PITX2, RUNX3, NFKbeta1, ACTG1, CDH1, DUSP4 or FARP1. More specifically, the nucleic target gene region is one or more of chr17:77042327-77043930, chr19:40715824-40716843, chr7:27109607-27110104, chr4:111761312-111764113, chr1:25127915-25131792, chr17:77042426-77043830, chr17:77080311-77081236, chr17:77092731-77097121, chr17:77109501-77110986, chr17:77042426-77043830, chr16:67328436-67329945, chr8:29261385-29265966, chr13:97592201-97594442, chr4: 103640925-103642461 or chr4: 103641494-103642135.

In some embodiments, the at least one primer that hybridizes to a strand of the nucleic acid target gene may have the forward primer sequence TTGGTTGTTTGGTAGGGGTAGTTAT (SEQ ID NO: 1), TGAAATGTTTTTAATGATTTAGTTGATG (SEQ ID NO: 2), GGGGTGTTGTAGAATTTTTTTTAGTTTAA (SEQ ID NO: a), GGGGTTAGGGTTTATTTTTGGGTA (SEQ ID NO: 4), TTGTTAATGGTGATGATTTGGTTAT (SEQ ID NO: 5), GGAAGTTGGGATTTGAGTTGGTTT (SEQ ID NO: 6), TTTTTTTTGGTTTTGTTTTGGTTTG (SEQ ID NO: 7), GGGAGTGGTTGAAATTTAAGTTGAG (SEQ ID NO: 8), GGTTTTGTTGTTGTAGATTTGTTTTATTTA (SEQ ID NO: 9), TTTTTGTGGGTTTTAGAGAAAGTTT (SEQ ID NO: 10), GGGGAGTTTTTTATTTTAATTGGG (SEQ ID NO: 11), TTTATTTTTAGGGAAAGAGGGAGGG (SEQ ID NO: 12), AGGGAGGTGGGTAGTTTTGTAGGAG (SEQ ID NO: 13), GGGTTTTTTTTATTGTAGGTTGAAGGTAT (SEQ ID NO: 4), GTTGGGGAGGATTTAGAGGGAGAT (SEQ ID NO: 15), TTTGGATTTTGTGGTTGTTTTTTTT (SEQ ID NO: 16), AAGTTGGAGGAGTAGGTTTAGTAGATA (SEQ ID NO: 17), CATCCAGAGGAGGTCTGTGTGGTGTG (SEQ ID NO: 18), GGTGTTTAGAGAAATTTTAGAAAGTTGGAT (SEQ ID NO: 19), TTTTTTAGGATATAGGTTATTTTTTGAAGG (SEQ ID NO: 20), TTTTTTTGATTTATTTTGAGGTTTT (SEQ ID NO: 21), GGGAGATAGAATTTATTTGGTTTATTTATA (SEQ ID NO: 22), TTAGGAGTGTTTGGGTATGGTTAGTA (SEQ ID NO: 23), GATTGGGTTTGAATGTAATTGAAAG (SEQ ID NO: 24), GTTAGGGGTTTTTTTTGTTTTTTTT (SEQ ID NO: 25), GGATTGGTGGGAAAATAAGAGAGTAGATT (SEQ ID NO: 26), GATTTTTTTTGTTTTATAGGGGGATT (SEQ ID NO: 27), CCCTGAGGCAGAGGGTGAGGAGTAG (SEQ ID NO: 28), TGTTTTTTAAATTTTTTGGAGGGAT (SEQ ID NO: 29), GGTTGAATGTTAGTTTTGAATTAAAAGT (SEQ ID NO: 30), TAATGGTAGGGTTGGGAAGGTGTATATTA (SEQ ID NO: 11), GGGACTCTCTGTGTGGTGCTGACAG (SEQ ID NO: 32), GGGTTGGAAAATTTTTTTTATAATTATTTT (SEQ ID NO: 33), TTGGGGGAGTTTTATTTTTGGAGAT (SEQ ID NO: 34), AAGGGTTTTTGTTGAAGTGGGTTAT (SEQ ID NO: 35), CATGTAGACTCTTTGTGGCTGGGGAG (SEQ ID NO: 36), TGTGTATTTGGATTAATTGTTATATAGTTT (SEQ ID NO: 37), GGGTTTTTATATATTTTTTAGGGGAATTGA (SEQ ID NO: 38), GTTAGGAATGTGGTTTTGGGGATT (SEQ ID NO: 39), TTTTTTTTGGGGGTTTTTTTGTGT (SEQ ID NO: 40), GGGAAGGGGATATATGAGGGATTTAT (SEQ ID NO: 41), GGGGTGGTAGTTAGAGAGTTTGAGAG (SEQ ID NO: 42), GGGTTGGAATTTAGTTTTAGTTTTGTTGT (SEQ ID NO: 43), GGGTATTGGAGAATAAAGATATTTTTAATA (SEQ ID NO: 44), GGGGGTTTTTAGTTGATAGAGGG (SEQ ID NO: 45), TTGTTGTTTGGGAGGGAGGT (SEQ ID NO: 46), TGTTGTGATTTGGGAGAGGTTTAAG (SEQ ID NO: 47), TTTTTATATTAAAGTATTTGGGATGGTTTT (SEQ ID NO: 48), GGGAGATTAGTATTTAAAGTTGGAGGTT (SEQ ID NO: 49), GGTATTTTAGGGGAAGTTGGTATTTTG (SEQ ID NO: 50), AGTGTTAGGAATTTAGATTTTGGTAAT (SEQ ID NO: 51), GTTTAGAGAGAGGGATTGGAGGTTTAGA (SEQ ID NO: 52), GTTTTTTGTAGTTGTTTGTTGGGTTTTG (SEQ ID NO: 53), TTTTTTGTTTGTTAGGGTTTTTTTT (SEQ ID NO: 54), TGTATTTTTTAATGGTTGGTTTGTTT (SEQ ID NO: 55), TTGGTTTAGGGTAATAGGGGTTTTG (SEQ ID NO: 56), TGATTTTTATAGAGTATGGGTGGG (SEQ ID NO: 57), ATTAGAGTATGATTTAGGTTTTTGATAGTT (SEQ ID NO: 58), GTTGTAGGTGGTTTTTTTAAGGATG (SEQ ID NO: 59), TAGGATTTTGTTGAATGAATGATTGAATT (SEQ ID NO: 60), GGGGAGGAGATTATTTGGTTTTTTT (SEQ ID NO: 61), GGGACCTGGGAAGGAGCATAGGACAG (SEQ ID NO: 62), GGGTTTAGGGGGAGGAGATTTAG (SEQ ID NO: 63), GAGGAGAGTTTTTTGGGGAAATG (SEQ ID NO: 64), GTAGGTAGTGTGTTAGGAAGGGGGT (SEQ ID NO: 65), GATTGTTTTGGGGTAATAAAAAGATT (SEQ ID NO: 66), TGGGAAAGAGGGAAAGGTTTTTT (SEQ ID NO: 67), GTAGTTGGGGGATGTTTGGATTT (SEQ ID NO: 68), AGGGTTTTGGGGATTTATTGGAG (SEQ ID NO: 69), TTTAGGTTAGTTGGGGTATTTTGGG (SEQ ID NO: 70), TTTTTTTTTAGTGTTTAGTTTAGAGTTTG (SEQ ID NO: 71), TGGTTGATATTTTTTGTGTAAAATATGTTG (SEQ ID NO: 72), GGGTATTATTGGTTTAATGGGGAAG (SEQ ID NO: 73), TTTTTTTTGTAGTTATTTTAGGGGAAGTAA (SEQ ID NO: 74), TTTTAGGTTTGGAGGTTGGTTAGGT (SEQ ID NO: 75), TGGATTTTTTTTATTTAGGGGTATA (SEQ ID NO: 76), TTAGAATGGAAGGGTAAGAGGTTTAAAT (SEQ ID NO: 77), TTTTTTTTATTAATTGGAGGAGAATTATAA (SEQ ID NO: 78), TTTAGGGTTTTAGTGGTGGTTATTAT (SEQ ID NO: 79), GAGAGAATTTTGTAGGTTAGGGGAGAG (SEQ ID NO: 80), GAGAGAATTTTGTAGGTTAGGGGAGAG (SEQ ID NO: 81), GAAGGTTGGTTTTGGTTTTTGAGTAGA (SEQ ID NO: 82), TTAGTTTTTAGGGAGTTTGGAGT (SEQ ID NO: 83), GAGTGGGTGGGTTTAGTTAGGTTTG (SEQ ID NO: 84), TATTAGGGGGTTTAGGGGTTGGTT (SEQ ID NO: 85), GGTAGAGTAGAAGGGTTTTTGTTTTTT (SEQ ID NO: 86), TTTTTAGGGGGAAGGGAGGTTT (SEQ ID NO: 87), TTTAGGTAGAGGAGTGGATTGGAGT (SEQ ID NO: 88), GAGGTTATTAGGTGGGATTTTTTGAG (SEQ ID NO: 89), TTGGTTGGGTTGTTGGAAGGT (SEQ ID NO: 90), GGGGTGTTGTAGAATTTTTTTTAGTTTAA (SEQ ID NO: 91), TATTTTGTTTAGGTAGGAGGTTAGG (SEQ ID NO: 92), TTTTTAGTTTAGGTGGGATTATATGGT (SEQ ID NO: 93), TTTTGGATAAGGGAAGTTGTGTATT (SEQ ID NO: 94), TTTTAAAGGTTTTTGGGTAGTGATT (SEQ ID NO: 95), GTTTTTTGTGGGTGTGGTTTTTTA (SEQ ID NO: 96), TGGTGTTTTATAGGTATTTGGGTTGTG (SEQ ID NO: 97), TGGAAAGTTTTGATTTTTTTGAGTTT (SEQ ID NO: 98), AGTTTGTTAAGTTTTATTGGGTTTTAGTT (SEQ ID NO: 99), GGAGTATATAGAAGTTGTAGGTTAGGAGGT (SEQ ID NO: 100), GGGTCCTGACCTTGATTCCTGCCACAG (SEQ ID NO: 101), TGGGTTTTTGTATAGATTAAAAATAAAAA (SEQ ID NO: 102), GGTAGTTTTTGTTTGAAATTTTAGTTTT (SEQ ID NO: 103), ATATTTATTTGGTGTTGGGTGTGGG (SEQ ID NO: 104), GATGGTTTAAGATTTATTTGTTGGGTAGGT (SEQ ID NO: 105), GTTGGTTTGGGGGTTTTTGATTAG (SEQ ID NO: 106), GGGCCTGTCTTCAGAAGAGAAAATGG (SEQ ID NO: 107), TTGTTTTTTTATGGAAATGAAGGATT (SEQ ID NO: 108), GAGATGTTGGTTTTTGTGGGAAGTT (SEQ ID NO: 109), GGGGATAGAGGAGTATTGAAAGTTAGTTTA (SEQ ID NO: 110), CCTCAGATTGAGGTCCCAGGGCCAAAGGA (SEQ ID NO: 111), GTAGAATTGGGGATTTTTGGTGT (SEQ ID NO: 112), TTTAAATAAAGTAAAGGAATGGGTTTT (SEQ ID NO: 113), TTAGTGGGAATTTTTAGTTAGGAAGTGAG (SEQ ID NO: 114), TCAGTGGGAATTTCCAGCCAGGAAGTGAG (SEQ ID NO: 115), TTTAGGGTTATTTAATTATAGGGTTAGTTA (SEQ ID NO: 116), TGGGGATTGAGGTTGGTTATTAATT (SEQ ID NO: 117), GGAGATTGGGAGGAATAATTTTTTTT (SEQ ID NO: 118), TGTTTTTTAAATTTTTTGGAGGGAT (SEQ ID NO: 119), GAGTTTTAGGGTTTGATGGGAA (SEQ ID NO: 120), TGGAGTGGGTAAGATCATTGCAAGCATGAC (SEQ ID NO: 121), GAGGGTATTATTTTTTGATAGGAAGAG (SEQ ID NO: 122), ACAAAGCTGGGTTCCTGCTGGGCCC (SEQ ID NO: 123), TTAGTTTTTTAATTTGTTTTGGGGGATAT (SEQ ID NO: 124), GGTGTGTATTTTTAGTTTGTGTTTGGAG (SEQ ID NO: 125), GGAAGATTTTTTAGGTTAAGTTGGAGA (SEQ ID NO: 126), TTGTTTTTTTATGGAAATGAAGGATT (SEQ ID NO: 127), AGTTTAGGTTGATTTAGAATAGGATTTTG (SEQ ID NO: 128), CCTGCCCTTGGCTGGGTAATCTCTG (SEQ ID NO: 129), ATGGATTTTAGGAATTTGTTTAAGGTTAT (SEQ ID NO: 130), TTTATTTGTTTTTTTGGTAGTTATAGAGTA (SEQ ID NO: 131), TTGTTGATGTTATATTTTTAGGTTTTAATT (SEQ ID NO: 132), GTTTGGGATTGTTTTGGAGGTATAG (SEQ ID NO: 133), TTGTTTGTTTTTGTAGGGTTGTTGG (SEQ ID NO: 14), GTTGTTTTTTGGTTGTTTTTTT (SEQ ID NO: 135), TTTAATTTGTAGTTTGGGGGTTGTTTT (SEQ ID NO: 136), ATTTTTTTAGGTAGGTGGTGGGGAA (SEQ ID NO: 137), GAGGGGAAAGGGTTTTATTTTTTTT (SEQ ID NO: 138), TTTTTTTTAGGTTTGGAGGGTTTTTG (SEQ ID NO: 139), GGGAGAGTTGGTTTTTATTTATTT (SEQ ID NO: 140), GTGAGGGTTTTGATTTTAGAATTAA (SEQ ID NO: 141), AAAGAAGTTTTGAGAATGTTTTTTTT (SEQ ID NO: 142), GGTTTTTAATTTTTTTAGGGAGGGG (SEQ ID NO: 143), CTGGTGACAGCCAGGTAGGTGGAAGTTT (SEQ ID NO: 144), TATATGGAGGTTTTGTTTTGTTTTAAAAA (SEQ ID NO: 145), AGGGAAGAAGTGACCCTGGCTGATG (SEQ ID NO: 146), GTGGGAGTTGTTGGTTTGAAATAAG (SEQ ID NO: 147), TTTTTTTGGGTAGTAAAGTGTTGGG (SEQ ID NO: 148), TTTTTTTGGGTAGTAAAGTGTTGGG (SEQ ID NO: 149), GCTGTGGGGTGGGGCACACTTG (SEQ ID NO: 150), TGGGGGTTTAGAGGTATAGTTTTTT (SEQ ID NO: 151), GGGTTTGGGGTTTAGTTTGTTTTG (SEQ ID NO: 152), TTTTTTGTTAGGTAGGTTTTAGTTATTGT (SEQ ID NO: 153), TGATGTAAGGATGTAGGGATTTAGAGATTA (SEQ ID NO: 154), TCTGGCTGTGGGGGACCAGGAC (SEQ ID NO: 155), TGAGTAATAGGGAGGGTTTTGGATTT (SEQ ID NO: 156), GGGGATTTTTAGGAATTGTAGGAG (SEQ ID NO: 157), AGATTTTTTTAGGAGGTTATAGAAGGTGTT (SEQ ID NO: 158), AGTAAGTTAGGAGGGTAGTGGGTGG (SEQ ID NO: 159), TTTTTTGGGTTGTTTTATTTTGTTT (SEQ ID NO: 160), CTGGGGCCCTCTGAGAGCAGGCAGGC (SEQ ID NO: 161), GTAGAGGGGGAGTTATAGGTGATGG (SEQ ID NO: 162), GGAGGGGAGTTTATTTATTTTTTTAATTTT (SEQ ID NO: 163), TTTTTAGAATTTTGTGTGTGTGTGTGTA (SEQ ID NO: 164), AAATTTTGTTGTATTGAGATATTTTAATGT (SEQ ID NO: 165), GGATGGGGAAACTGAGGCTCCAAGCA (SEQ ID NO: 166), TGTATAAAGTAGAAATTTAAATGTTAGGG (SEQ ID NO: 167), TTTAGGGAAATAAAATGGAAATTTTA (SEQ ID NO: 168), GGGTGTTCCCTGGCAGAGAGGCTCT (SEQ ID NO: 169), TAGGATTTTGTTGAATGAATGATTGAATT (SEQ ID NO: 170), GATTGTTTTGGGGTAATAAAAAGATT (SEQ ID NO: 171), GAATTAGGGGGAGGGGTTGTTT (SEQ ID NO: 172), AGTTTTTTTGTTTTTAGTTTGGTTTTGTTA (SEQ ID NO: 173), AGTTTGATTTTTATTTTGGTGTAGTTT (SEQ ID NO: 174), TGGTGTTGTGGTTGATGTATTTTATG (SEQ ID NO: 175), GAGGTGGTGATGTTTAGGGTTAGAG (SEQ ID NO: 176), GGGTAAGTTTGGTATGGTGTTGTTG (SEQ ID NO: 177), AAGTTTTTGAGAAATTTTTTTAAAAATTGT (SEQ ID NO: 178), GATGGTGTTAGGTTTTTGGTTTGG (SEQ ID NO: 179), or the reverse primer sequence (SEQ ID NO:), ACTAACCACTTTTTCTTTTATAACTTTCAT (SEQ ID NO: 180), ATCCCATAATAACTCCCAACTTTAC (SEQ ID NO: 181), AAAATCCTTATCCCCCATAAACAAC (SEQ ID NO: 182), TCTAAACTACTCCCTCCCCAAATCC (SEQ ID NO: 183), TCCTCCCTAAAACCTCCAAATTTCT (SEQ ID NO: 84), CTCCCCAAACAACCCTACCTCTAT (SEQ ID NO: 185), CTCAACCTCCATTTTCTCCTCTAAAC (SEQ ID NO: 186), TTCCAACACCCAAATCTACTTCCTC (SEQ ID NO: 187), TCCTTAAAAACCAAAAACTCCTCCC (SEQ ID NO: 188), AAAACAAACAACTCCCAACACTAC (SEQ ID NO: 89), CTCCAAACAAAACTACCTCCAACTC (SEQ ID NO: 190), AAAACTACCCCAAACACACTTCCC (SEQ ID NO: 191), ACAAAACAAAAACACCCTCATAACC (SEQ ID NO: 192), TCTAATATAAACCCCTACCCCCTCC (SEQ ID NO: 193), ATAAACAACCCACACCAAAACAACC (SEQ ID NO: 194), CCCTTTAAACCTTTTACAATCCTAAC (SEQ ID NO: 195), AAACTAAAATCCACCCCAAAAAAAC (SEQ ID NO: 196), GGCTGTCACACTGGGGCTGCTGCTCA (SEQ ID NO: 197), CTTCCAACCTCAACAAAAAATAACC (SEQ ID NO: 198), CATAACACAACCCAACTTCACCAAC (SEQ ID NO: 199), AAACCCCAAACAACTACACACCTAAC (SEQ ID NO: 200), AATCCTACCTCTACTTCCTCCCAAC (SEQ ID NO: 201), CCCCTCCCCTCAACTTAAAATTAAA (SEQ ID NO: 202), AAAAATATATCCCTCCCAAAAACCC (SEQ ID NO: 203), AATACTTTATCTCTACAACAAAACTACCC (SEQ ID NO: 204), TACCAAAACCTAAAATACCAACAAC (SEQ ID NO: 205), CTAAAAACTCCCAACCCTAAAAACC (SEQ ID NO: 206), GCCTGCCTGGGCCTGCTGGCAGTG (SEQ ID NO: 207), AAAAAAAACCATACTTTCCCTATAACACCA (SEQ ID NO: 208), AAATAAAAATAAACTAAACACAAAAAACTC (SEQ ID NO: 209), AATCCTAACTCCCAAAAACCCACTT (SEQ ID NO: 210), GTGACCCTGGGAAATGCTTCTATCCCTG (SEQ ID NO: 211), CACTCAAAAAACCCCAAAACCTAAC (SEQ ID NO: 212), CTACAAACTACACAACCCTCCAACTC (SEQ ID NO: 213), ACCTATAACTAAAAAACCCCCAAAC (SEQ ID NO: 214), AGGAGGAGGGAAGGGAGTCCACCCC (SEQ ID NO: 215), ACCTTTACCCCCAATACCTACCTC (SEQ ID NO: 216), CCAAAAACTAACCCCACTACATCAAC (SEQ ID NO: 217), TCAATCTCCAATCCTTTTAAAAAAAA (SEQ ID NO: 218), ACCAATCCCTATAACCCCCTCC (SEQ ID NO: 219), CCAAAAACCACAAACAACCTTAAAC (SEQ ID NO: 220), AAACAACAAAAAAACCACCTAAATC (SEQ ID NO: 221), TTACTCCTCCAAATAAACCCAATCC (SEQ ID NO: 222), TCAAAACCAAAATAACAAAACTCC (SEQ ID NO: 223), TAACCCAAAAATACAAATTTTCAAC (SEQ ID NO: 224), AAAAAAATTCCCACTTTAAAAAAAC (SEQ ID NO: 225), CCCACCTACTAAATAAAACCCAAC (SEQ ID NO: 226), TCCAAATAATAAAACACCTACTAACC (SEQ ID NO: 227), AATCTAATACAATAAAACCATCCCAAATAC (SEQ ID NO: 228), ACCTATACACCCAACCTACACACCC (SEQ ID NO: 229), AAAAAACTCCTCACTTTAAAAAAAA (SEQ ID NO: 230), AAAAAACAATCTTCAAAAACCCACC (SEQ ID NO: 231), AAACACTATTATCCCCCATTTACAAATAAA (SEQ ID NO: 232), AATAAAACCTTCCTTTAATCCCCTCC (SEQ ID NO: 233), CCCTCTTCCTCCCCTACTAATCCTAC (SEQ ID NO: 234), CCAAACCAATAAAAAATCTCCCAAC (SEQ ID NO: 235), AAAACCCATAAAAACCACAACCC (SEQ ID NO: 236, AAAACTAACATTTTCAACAAAAACTC (SEQ ID NO: 237), AAAACCCTACCTATTTTTCTTAATCCC (SEQ ID NO: 238), TTTAAAAACCACCTAACCCCAAATC (SEQ ID NO: 239), CCCCAAAACTTTAATCCTATCTCCC (SEQ ID NO: 240), GGCCATAACTAGGGAGGAAGGAGGGC (SEQ ID NO: 241), AAACAAATTCAACCCCAAATTCAAC (SEQ ID NO: 242), ACTCTTCCAAACCTTAAAAACCCC (SEQ ID NO: 243), CCAACCCAACCCAACAATAATAAAA (SEQ ID NO: 244), TAATCTCCCTCCAAAAATTCCAACA (SEQ ID NO: 245), CCCATACTAAAAACTCTAAACCCCATC (SEQ ID NO: 246), ACCCCTCACACCATTATCACTATCAA (SEQ ID NO: 247), AAAACAATTCTAACCCCACACATTTC (SEQ ID NO: 248), CTAACAAAACTCCAAACCAATCACC (SEQ ID NO: 249), AAAAAACAAACATCTTCTCTTTCCCTACTA (SEQ ID NO: 250), TCAAACAAAAAACCAATTCCAAATC (SEQ ID NO: 251), AAAAACCCAAAACCCTAATCCCTAC (SEQ ID NO: 252), AACAAACCACCAAACAAACACATC (SEQ ID NO: 253), AACCACTTTTTCTTTTATAACTTTCATATC (SEQ ID NO: 254), CTACAACAACCCCAACTCCCTC (SEQ ID NO: 255), AATCCAAAACTCACTAACAAAAATC (SEQ ID NO: 256), CCACTAAAACCCTAAACAACTACTAC (SEQ ID NO: 257), ACAAAAACAAAACTAAATTTAATCTTTTAA (SEQ ID NO: 258), CAAATCAAAATCTAATTTCAAAACC (SEQ ID NO: 259), CAAATCAAAATCTAATTTCAAAACC (SEQ ID NO: 260), CCCCACCCACAAAAAAATAAATAAAA (SEQ ID NO: 261), ATTACACAAAAAACTTAAACCAAAATCAAC (SEQ ID NO: 262), ACCCTCTCTCCCTCTATAAACCTCC (SEQ ID NO: 263), TAAACTCACTCCCCAACATAAAAAC (SEQ ID NO: 264), AAACAACCAATCAAATAACTAAATTTACCA (SEQ ID NO: 265), AAAAATCTCTCAAAAACCAATCAAC (SEQ ID NO: 266), ATCCTAAATCTCACCTAAAACCCC (SEQ ID NO: 267), ACCCCCAACTACTATCCCTCACTAC (SEQ ID NO: 268), AAAAAAAACCTCCTCCCACAAAAAA (SEQ ID NO: 269), AAAATCCTTATCCCCCATAAACAAC (SEQ ID NO: 270), CAAATTCCTCAAAACTCAAATATCC (SEQ ID NO: 271), ATACAACTCAAAAAACAATACCTCATTCAT (SEQ ID NO: 272), AAAAACTTCAACCACCAAAAAAC (SEQ ID NO: 273), ACAACCTAACACCCCACTTTACCAT (SEQ ID NO: 274), AACCCAAAAAATCTCTCCAATTACC (SEQ ID NO: 275), TCCCCCTCAAAAAAATTTAATTCATAAA (SEQ ID NO: 276), CCCATTCCAACTACCTAACCCC (SEQ ID NO: 277), AAAAAAACAAACTACCTTTCCTCCC (SEQ ID NO: 278), CAAAACCTCTCCCAAAATCTCAAAC (SEQ ID NO: 279), GCAGGGGTGGAACTGGATTCTGC (SEQ ID NO: 280), AAACCAAACCAATAACCAAAAAATC (SEQ ID NO: 281), AATCTAAACTCCCCCACCTCCTAAC (SEQ ID NO: 282), AAAAATAACCTCCTTACCAATCAAAACC (SEQ ID NO: 283), AACTTCCTTCAATCATCCAATCTTTATTC (SEQ ID NO: 284), CCTTTTCCTATCACAAAAATAATCC (SEQ ID NO: 285), GGAAGGCTGAACTGCTGAGTCTGAC (SEQ ID NO: 286), CCCCAATCCAACCTAAACTCTAAAC (SEQ ID NO: 287), AAATCACACAAACCTCCTCATTAACTACT (SEQ ID NO: 288), AAAACTTCCTCACCCCTAACTTCTC (SEQ ID NO: 289), GAGGTAGAATGGATCCCCTTGGCCTTC (SEQ ID NO: 290), CAAAATAACTCCCTCCAAACAAAAC (SEQ ID NO: 291), AATATATTCTCCCATCTATCTCACTCAAA (SEQ ID NO: 292), CCTCTCAACTACTATCAACCTCCTCC (SEQ ID NO: 293), GGAGGAGGTTGACAGTAGCTGAGAGG (SEQ ID NO: 294), AAAAAAACTAAACCACCAAAAACCC (SEQ ID NO: 295), CTCCCAAATTCTCTAAACCCCAACT (SEQ ID NO: 296), CTCCCTAACACCTAAACTCCCAAAC (SEQ ID NO: 297), AAAAACCCAATCCTCCTTCCTTAC (SEQ ID NO: 298), CCAATTCTTTTAAAAACACTATATTCCTTA (SEQ ID NO: 299), TAGGTCTGCAGAGTGGTCTTCCTGGTA (SEQ ID NO: 300), CAAACTACCAATACCACTCACTCACTAC (SEQ ID NO: 301), GGAGCAGCACCCTTCCAGGGGAGGTGG (SEQ ID NO: 302), TTTATCAAAACATCATTTTCTCCCTATAA (SEQ ID NO: 303), AATACCCTTCTACCCACATCCCATAT (SEQ ID NO: 304), AAAAACCACTACCTTAACTCCCCTC (SEQ ID NO: 305), AACAAACCCCCTCTCCCTACTACC (SEQ ID NO: 306), CCCCTAAAACAAAAATAATAACCAAC (SEQ ID NO: 307), GTCTGGGGCCAGCAGGGGGCACTA (SEQ ID NO: 308), ATAAAAAATCTACCCCAACCCCTTC (SEQ ID NO: 309), CACAATCCAAACAAAAAACCCTC (SEQ ID NO: 310), CTAAAAAACCCCACACCCCAAC (SEQ ID NO: 311), AAAAAAACAAACAAATAACCTACCTCTCAC (SEQ ID NO: 312), AATTCCCAAAAAAATCCCAAATTCT (SEQ ID NO: 313), ATCCCTACACCCAAATTTCCATTAC (SEQ ID NO: 314), TAACCAACTAACTCCAATCACTCCC (SEQ ID NO: 315), CCTCAAAACCCAACAAACTCAAACT (SEQ ID NO: 316), AACCTACTTCATAACCCTAATCATC (SEQ ID NO: 317), AACTACAAAAAATTTTCCCACTTCCC (SEQ ID NO: 318), ACCCACAAAAATCCCTCATTCTCTA (SEQ ID NO: 319), CTACCTCCACCTCACTCTTAATAAC (SEQ ID NO: 320), TTCCAAACACACTTTATATAAAATCTACAA (SEQ ID NO: 321), CCCACCAATAACTCCTCCTCCTACT (SEQ ID NO: 322), CCACTTTGGGTCTAGGGAGAGGAGG (SEQ ID NO: 323), AAATAAACCAACCCTTACCCAATCTC (SEQ ID NO: 324), GGGTGGATCACCTGAGGTCAGGAGT (SEQ ID NO: 325), CCCTAACTTTATCTCTCTATAAATACACC (SEQ ID NO: 326), AAAACTCAAAAAACTTATCTTTAAAACACA (SEQ ID NO: 327), AAAACTCAAAAAACTTATCTTTAAAACACA (SEQ ID NO: 328), TTACATGGAGGACCTGCAGGAGCTCACCAT (SEQ ID NO: 329), CCACACCTATCTAAACACCAAAATC (SEQ ID NO: 330), TAAAAACTCCAATCCAACTTTCCAC (SEQ ID NO: 331), CCAAACTACCAAATCCCCCTACTC (SEQ ID NO: 332), TCAAACCAACCCTAATACACTACCC (SEQ ID NO: 333), AAAGTGGGCTCCACTAAGCTGGGAAGG (SEQ ID NO: 334), AACTAAAATAACTAACAACCCAAATAAATA (SEQ ID NO: 335), CCATACCCAAAAAAAACTAACTAAACC (SEQ ID NO: 336), TCCAAATCCAAAACTCCCAATCTAC (SEQ ID NO: 337), TATCACCCCAAAAAAACTATCTCCC (SEQ ID NO: 338), AACAACAAAATCTTCTTTCCCCATC (SEQ ID NO: 339), CCAGTGGATGGGCCTGGTTTGTTCC (SEQ ID NO: 340), CCCCCAACCAAAACTAAAAAAAAC (SEQ ID NO: 341), ACCTCTTAATCCCCTCCCTATTATACC (SEQ ID NO: 342), AACAAAACATCCTATCCAAACATCC (SEQ ID NO: 343), AAAACTAATACCAAACAAAAACCCC (SEQ ID NO: 344), GACCTGGGAGGCCACCCATTGCCCA (SEQ ID NO: 345), CACAAATTTAATCTCCATTCTCCTC (SEQ ID NO: 346), CATAAAAATCAATAAATAACCCCAC (SEQ ID NO: 347), GCCCAAGAAGATTGTAAATGCCAAGAAAGG (SEQ ID NO: 348), TTTAAAAACCACCTAACCCCAAATC (SEQ ID NO: 349), TAATCTCCCTCCAAAAATTCCAACA (SEQ ID NO: 350), AAACCATCTTCCTCCCCTACAAAA (SEQ ID NO: 351), AAAAAAAATCCCTACACCACCTCC (SEQ ID NO: 352), AATACAAAAAACACAACCCCTACAACC (SEQ ID NO: 353), CAATCTCCTTTAACCTAACTAAACAATC (SEQ ID NO: 354), TCCAAATTTTAACAACTCCAAAACC (SEQ ID NO: 355), ACTTAACCTTCCTACTCCCCCTCC (SEQ ID NO: 356), AAACAAACAACCTCCCCACTTACAT (SEQ ID NO: 357), or CCTAAATTCTCCCTAAACCCCTCCTA (SEQ ID NO: 358)

In certain embodiments the primer sequence further comprises a promoter sequence. In an embodiment the promoter sequence is obtained from a T7 promoter, a SP6 promoter or a T3 promoter. If the promoter is a T7 promoter it may have the sequence: 5′-CAGTAATACGACTCACTATAGGGAGA-3′ (SEQ ID NO.: 359)

In an embodiment, where the nucleic acid target gene region is the IGF2/H19, the primers may have the sequences: 5′-CAGTAATACGACTCACTATAGGGAGAAGGCTGTTAGTTTTTATTTTATTTTTAA-3′ (SEQ ID NO.: 360), 5′-AGGAAGAGAGAACCACTATCTCCCCTCAAAAAA-3′ (SEQ ID NO.: 361), 5′-AGGAAGAGAGGTTAGTTTTTATTTTATTTTTAAT-3′ (SEQ ID NO.: 362) or 5′-CAGTAATACGACTCACTATAGGGAGAAGGCTAACCACTATCTCCCCTCAAAAAA-3′ (SEQ ID NO.: 363).

In some embodiments, a data structure of a nucleic acid target gene region for predicting disease outcome of a subject that correlates with changes in the methylation state of a subject's tissue or cell is provided comprising, a first data set providing the characteristic methylation state of at least one known subject with a good outcome, a second data set providing the characteristic methylation state of at least one known subject with a poor outcome, a third data set of an individual's characteristic methylation state, and providing a comparison of the individual's characteristic methylation state with the first and second data sets. In addition either the first data set or the second data set of the data structure may provide the methylated/unmethylated ratio for each methylation site of a nucleic acid target gene region of the subject with a good outcome.

In a related embodiment, another data set is a representation of the first and second data sets as a hierarchical cluster.

In certain embodiments, data sets comprising the characteristic methylation state of a nucleic acid, nucleic acid target gene region or gene obtained by any of the methods described herein is provided. A characteristic methylation state of a nucleic acid target region determined by spectral analysis of base-specifically cleaved amplified nucleic acid target gene region that has been treated with a reagent that modifies unmethylated cytosine to produce uracil is provided. A characteristic methylation state of a nucleic acid target gene region identified by any of the methods described herein is provided, as well as the characteristic methylation state of a nucleic acid target gene or nucleic acid target gene regions listed above identified by any of the methods described herein is provided.

In some embodiments, a method is provided for identifying at least one CpG island region in a nucleic acid having a characteristic methylation state that correlates with an unknown disease outcome of an organism, tissue or cell comprising the steps of providing a first CpG island region of the nucleic acid; identifying or discovering at least a second CpG island region within a region spanning about 5 Kb 5′ of the first CpG island region and about 5 Kb 3′ of the first CpG island region in the nucleic acid including the first CpG island region; and determining if at least one of the at least a second CpG island region has a characteristic methylation state that correlates with the unknown disease outcome of the organism, tissue or cell.

In the preceding embodiments, the methylation state of 50 or more gene target regions in the nucleic acid of the subject is determined in 24 hours or less. In some embodiments the methylation state of 50 or more gene target regions in the nucleic acid of the subject is determined in 12 hours or less, 8 hours or less, 6 hours or less, 5 hours or less, 4 hours or less, 3 hours or less, 2 hours or less, or less than 1 hour. In some embodiments the methylation state of 100 or more gene target regions in the nucleic acid of the subject is determined in 24 hours or less. In some embodiments the methylation state of 100 or more gene target regions in the nucleic acid of the subject is determined in 12 hours or less, 8 hours or less, 6 hours or less, 5 hours or less, 4 hours or less, 3 hours or less, 2 hours or less, or less than 1 hour. In some embodiments the methylation state of 150 or more gene target regions in the nucleic acid of the subject is determined in 24 hours or less. In some embodiments the methylation state of 150 or more gene target regions in the nucleic acid of the subject is determined in 12 hours or less, 8 hours or less, 6 hours or less, 5 hours or less, 4 hours or less, 3 hours or less, 2 hours or less, or less than 1 hour. In some embodiments the methylation state of 20 or more gene target regions in the nucleic acid of the subject is determined in 24 hours or less. In some embodiments the methylation state of 20 or more gene target regions in the nucleic acid of the subject is determined in 12 hours or less, 8 hours or less, 6 hours or less, 5 hours or less, 4 hours or less, 3 hours or less, 2 hours or less, or less than 1 hour.

The methods, combinations and kits provided herein can be performed or used in conjunction with any of a variety of other procedures including, but not limited to, any procedures for modifying the target nucleic acid molecule according to the methylation state of the target nucleic acid molecule, any procedures for amplifying a target nucleic acid molecule, any procedures for fragmenting a target nucleic acid molecule, and any procedures for detecting target nucleic acid molecule fragments.

### BRIEF DESCRIPTION OF THE DRAWINGS

FIG. 1A displays mass signals generated by cytosine specific cleavage of the forward transcript of the IGF2/H19 region (upper spectral analysis is the methylated template; lower spectral analysis is the non-methylated template) (SEQ ID NO: 364). FIG. 1B shows the IGF2/H19 RNA transcript sequence wherein each CpG sequence is methylated (upper sequence) and the same RNA transcript sequence where none of the CpG sequence is methylated (lower sequence) (SEQ ID NO: 365).

FIG. 2 is an overlay of mass signal patterns generated by cytosine specific cleavage of the forward transcript of the IGF2/H19 region.

FIG. 3 is an overlay of mass spectra generated by uracil specific cleavage of the reverse transcript of the IGF2/H19 region (SEQ ID NOS 366 & 367).

FIG. 4 depicts mass spectra representing all four base-specific cleavage reactions of the IGF2/H19 amplicon. Numbers correspond to the CpG positions within this target region. Arrows point at the mass signals that indicate the presence of a methylated Cytosine at the marked position. All methylated CpG's in the selected region can be identified by one or more mass signals.

FIG. 5 depicts mass spectra generated by uracil specific cleavage of the reverse transcript of the IGF2/H19 region. Genomic DNA was used for amplification. Dotted lines mark the position of mass signals representing non-methylated CpG's. Signals with 16 Dalton shift (or a multitude thereof) represent methylation events. The area-under-the-curve ratio of methylated versus non-methylated template approximates to 1, as one expects for hemi-methylated target regions.

FIG. 6A is a hierarchical cluster analysis of 96 diagnostic AML samples. More specifically, FIG. 6A is an overview of a two-way hierarchical cluster of 96 AML samples (rows) and DNA-methylation of 180 genomic regions (columns). The names of the CpG sites that were analyzed can be found in Table 9, where the units in the table are oriented from left to right. For example, X053_KIAA1447_{—}01_CpG 2.3.4 corresponds to the far left column and X015_CD3D_{—}01_CpG_{—}25.26.27 corresponds to the far right column of the histogram in FIG. 6A. Also, a sample ID for the AML samples is provided along the y-axis of FIG. 6A and can also be found in Table 10, where the samples in the Table are oriented from bottom to top. For example, sample ID 103_{—}02KM1932 corresponds to the bottom row and sample ID 027_AML_{—}087 corresponds to the top row of the histogram in FIG. 6A. DNA-methylation values are depicted by a pseudocolor scale (indicated). Gray denotes poorly-measured data. b DNA-methylation variability across samples (distribution of value variance).

FIG. 6B are methylation results showing variable methylation ratios along the HOXA7 and DUSP4 genes.

FIG. 6C is a graph showing regression analysis, which reveals a strong correlation between the methylation ratios in peripheral blood (PB) samples and bone marrow (BM) samples

FIG. 6D is a histogram showing variance of the degree of methylation for each CpG unit was calculated to obtain a measure for the DNA-methylation variability across samples.

FIG. 7 is a qunatile-quantile plot that shows the most pronounced differences among samples occurred in CpG Units that are less than 50% methylated in the group of low DNMT expression.

FIGS. 8A-C are DNA-methylation-based outcome predictions in 192 AML samples. Kaplan-Meier survival analysis comparing the cluster-defined subset of samples predicted to have “good” or “poor” outcome (log rank test P-value is indicated) in a the training (n=89), b independent test set (n=93), and c validation set.

FIG. 9A-C are outcome predictions in 96 AML samples with available gene expression data. The Figures show Kaplan-Meier survival analysis comparing the cluster-defined subset of samples predicted to have “good” or “poor” outcome (log rank test P-value is indicated) based on a DNA-methylation analysis, b gene expression analysis, and c a combined predictor.

FIG. 10 is a flow chart showing the therapeutic options available to an AML patient based upon currently known prognostic factors.

Throughout the document and in the Figures, CpG sites are referenced according to their CpG ID. The CpG ID's refer to the specific CpG location within the particular genomic region. For example, each CpG ID follows the general schema: databaseID_GeneName_AmpliconID_CPG_CPGposition in the amplicon. “GeneName” is the refseq gene name of the analysed promoter region, or in the case of intragenic regions, the nearest gene is identified. “AmpliconID” is the particular amplicon analyzed within the gene or region, especially relevant if multiple amplicons were analyzed for this gene. “CPG” is a constant text string. “CPGposition in the amplicon” indicates which CpG Sites are enclosed in the measured CpG Unit. The numbers given refer to the CpG sites as counted from the 5′ end of the analyzed amplicon sequence. The amplicon sequences are provided in Table 8.

### DETAILED DESCRIPTION OF THE INVENTION

Unless defined otherwise, all technical and scientific terms used herein have the same meaning as is commonly understood by one of skill in the art to which the invention(s) belong. All patents, patent applications, published applications and publications, GENBANK sequences, websites and other published materials referred to throughout the entire disclosure herein, unless noted otherwise, are incorporated by reference in their entirety. In the event that there are a plurality of definitions for terms herein, those in this section prevail. Where reference is made to a URL or other such identifier or address, it is understood that such identifiers can change and particular information on the internet can come and go, but equivalent information is known and can be readily accessed, such as by searching the internet and/or appropriate databases. Reference thereto evidences the availability and public dissemination of such information.

As used herein, a “nucleic acid target gene region” is a nucleic acid molecule that is examined using the methods disclosed herein. For the purposes of the application, “nucleic acid target gene region”, “region” and “gene” may be used interchangeably. A nucleic acid target gene region includes genomic DNA or a fragment thereof, which may or may not be part of a gene, a segment of mitochondrial DNA of a gene or RNA of a gene and a segment of RNA of a gene. A nucleic target gene region may be further defined by its chromosome position range. The chromosome position ranges provided herein were gathered from the March 2006 human reference sequence (NCBI Build 36.1), which was produced by the International Human Genome Sequencing Consortium and can be accessed at http address genome.ucsc.edu/cgi-bin/hgGateway. A gene region can include one or more or a portion of the following: open reading frame, 3′ untranslated region, 5′ untranslated region, promoter region and enhancer region. A gene region can include a subsequence of a particular gene (e.g., KIAA1447), such as a methylated sequence (e.g., hyper-methylated sequence) therein.

In the context of methods for prognosis determination, the invention provides methods for identifying the methylation state of a nucleic acid target gene region and/or the methylation state of a nucleotide locus. A nucleic acid target gene region can also refer to an amplified product of a nucleic acid target gene region, including an amplified product of a treated nucleic acid target gene region, where the nucleotide sequence of such an amplified product reflects the methylation state of the nucleic acid target gene region. One skilled in the art would recognize that the size or length of the nucleic acid target gene region may vary depending on the limitation, or limitations, of the equipment used to perform the analysis. The nucleic acid target gene region may comprise intragenic nucleic acid, a gene of interest, more than one gene of interest, at least one gene of interest or a portion of a gene of interest. Correspondingly a sequential or non-sequential series of nucleic acid target gene regions may be analyzed and exploited to map an entire gene or genome. The intended target will be clear from the context or will be specified.

As used herein, a “nucleic acid target gene molecule” is a molecule comprising a nucleic acid sequence of the nucleic acid target gene region. The nucleic acid target gene molecule may contain less than 10%, less than 20%, less than 30%, less than 40%, less than 50%, greater than 50%, greater than 60%, greater than 70% greater than 80%, greater than 90% or up to 100% of the sequence of the nucleic acid target gene region.

As used herein, the “methylation state” of a nucleic acid target gene region refers to the presence or absence of one or more methylated nucleotide bases or the ratio of methylated cytosine to unmethylated cytosine for a methylation site in a nucleic acid target gene region. For example, a nucleic acid target gene region containing at least one methylated cytosine is considered methylated (i.e. the methylation state of the nucleic acid target gene region is methylated). A nucleic acid target gene region that does not contain any methylated nucleotides is considered unmethylated. Similarly, the methylation state of a nucleotide locus in a nucleic acid target gene region refers to the presence or absence of a methylated nucleotide at a particular locus in the nucleic acid target gene region. For example, the methylation state of a cytosine at the 7th nucleotide in a nucleic acid target gene region is methylated when the nucleotide present at the 7^{th} nucleotide in the nucleic acid target gene region is 5-methylcytosine. Similarly, the methylation state of a cytosine at the 7th nucleotide in a nucleic acid target gene region is unmethylated when the nucleotide present at the 7th nucleotide in the nucleic acid target gene region is cytosine (and not 5-methylcytosine). Correspondingly the ratio of methylated cytosine to unmethylated cytosine for a methylation site or sites can provide a methylation state of a nucleic acid target gene region.

As used herein, a “characteristic methylation state” refers to a unique, or specific data set comprising the location of at least one, a portion of the total or all of the methylation sites of a nucleic acid, a nucleic acid target gene region, a gene or a group of genes of a sample obtained from an organism, a tissue or a cell.

As used herein, “methylation ratio” refers to the number of instances in which a molecule or locus is methylated relative to the number of instances the molecule or locus is unmethylated. Methylation ratio can be used to describe a population of individuals or a sample from a single individual. For example, a nucleotide locus having a methylation ratio of 50% is methylated in 50% of instances and unmethylated in 50% of instances. Such a ratio can be used, for example, to describe the degree to which a nucleotide locus or nucleic acid region is methylated in a population of individuals. Thus, when methylation in a first population or pool of nucleic acid molecules is different from methylation in a second population or pool of nucleic acid molecules, the methylation ratio of the first population or pool will be different from the methylation ratio of the second population or pool. Such a ratio also can be used, for example, to describe the degree to which a nucleotide locus or nucleic acid region is methylated in a single individual. For example, such a ratio can be used to describe the degree to which a nucleic acid target gene region of a group of cells from a tissue sample are methylated or unmethylated at a nucleotide locus or methylation site.

As used herein, a “methylated nucleotide” or a “methylated nucleotide base” refers to the presence of a methyl moiety on a nucleotide base, where the methyl moiety is not present in a recognized typical nucleotide base. For example, cytosine does not contain a methyl moiety on its pyrimidine ring, but 5-methylcytosine contains a methyl moiety at position 5 of its pyrimidine ring. Therefore, cytosine is not a methylated nucleotide and 5-methylcytosine is a methylated nucleotide. In another example, thymine contains a methyl moiety at position 5 of its pyrimidine ring, however, for purposes herein, thymine is not considered a methylated nucleotide when present in DNA since thymine is a typical nucleotide base of DNA. Typical nucleoside bases for DNA are thymine, adenine, cytosine and guanine. Typical bases for RNA are uracil, adenine, cytosine and guanine. Correspondingly a “methylation site” is the location in the target gene nucleic acid region where methylation has, or has the possibility of occurring. For example a location containing CpG is a methylation site wherein the cytosine may or may not be methylated.

As used herein, a “methylation site” is a nucleotide within a nucleic acid, nucleic acid target gene region or gene that is susceptible to methylation either by natural occurring events in vivo or by an event instituted to chemically methylate the nucleotide in vitro.

As used herein, a “methylated nucleic acid molecule” refers to a nucleic acid molecule that contains one or more methylated nucleotides that is/are methylated.

As used herein “CpG island” refers to a G:C-rich region of genomic DNA containing a greater number of CpG dinucleotides relative to total genomic DNA. A CpG island may be about 200 base pairs in length, where the G:C content of the region is at least 50% and the ratio of observed CpG frequency over expected frequency is 0.6; typically a CpG island can be at least 500 base pairs in length, where the G:C content of the region is at least 55% and the ratio of observed CpG frequency over expected frequency is 0.65. The observed CpG frequency over expected frequency can be calculated according to the method provided in Gardiner-Garden et al., J. Mol. Biol. 196:261-281 (1987). For example, the observed CpG frequency over expected frequency could be calculated according to the formula:

R=(A×B)/(C×D)

where R is the ratio of observed CpG frequency over expected frequency, A is the number of CpG dinucleotides in an analyzed sequence, B is the total number of nucleotides in the analyzed sequence, C is the total number of C nucleotides in the analyzed sequence, and D is the total number of G nucleotides in the analyzed sequence.

As used herein, a first nucleotide that is “complementary” to a second nucleotide refers to a first nucleotide that base-pairs, under high stringency conditions to a second nucleotide. An example of complementarity is Watson-Crick base pairing in DNA (e.g., A to T and C to G) and RNA (e.g., A to U and C to G). Thus, for example, G base-pairs, under high stringency conditions, with higher affinity to C than G base-pairs to G, A or T, and, therefore, when C is the selected nucleotide, G is a nucleotide complementary to the selected nucleotide.

As used herein, “treat”, “treating” or grammatical variations thereof, refers to the process of exposing an analyte, typically a nucleic acid molecule, to conditions under which physical or chemical analyte modification or other chemical reactions (including enzymatic reactions) can occur. For example, treating a nucleic acid target gene molecule with a reagent that modifies the nucleic acid target gene molecule as a function of its methylation state may include adding a reagent such as bisulfite or an enzyme such as cytosine deaminase to a solution containing the nucleic acid target gene region. In treating the nucleic acid target gene with bisulfite any unmethylated nucleotide, such as any unmethylated C nucleotide, present in the nucleic acid target gene molecule can be chemically modified, such as deaminated; however, if the nucleic acid target gene molecule contains no unmethylated selected nucleotide, such as no unmethylated C nucleotide, then a nucleic acid target gene molecule treated with such a reagent may not be chemically modified. In another example, treating a nucleic acid target gene molecule under fragmentation or cleavage conditions can include adding a cleavage reagent such as RNase T1, such that in selected nucleic acid target gene molecules, such as nucleic acid target gene molecules containing G nucleotides, cleavage can occur. Cleavage, however, need not occur, such as with nucleic acid target gene molecules not containing G nucleotides, cleavage with RNase T1 may not occur. In another example, treating a nucleic acid target gene molecule under nucleic acid synthesis conditions can include adding a DNA or RNA polymerase and NTPs, such that nucleic acid synthesis can occur if, for example, a primer is hybridized to a nucleic acid target gene molecule, however, no nucleic acid synthesis is necessary if, for example, no primer is hybridized to a nucleic acid target gene molecule.

As used herein, the phrase “hybridizing” or grammatical variations thereof, refers to binding of a first nucleic acid molecule to a second nucleic acid molecule under low, medium or high stringency conditions, or under nucleic acid synthesis conditions. Hybridizing can include instances where a first nucleic acid molecule binds to a second nucleic acid molecule, where the first and second nucleic acid molecules are complementary.

As used herein, “specifically hybridizes” refers to preferential hybridization under nucleic acid synthesis conditions of a probe, or primer, to a nucleic acid molecule having a sequence complementary to the probe or primer compared to hybridization to a nucleic acid molecule not having a complementary sequence. For example, specific hybridization includes the hybridization of a probe to a target nucleic acid sequence that is complementary to the probe.

As used herein, “nucleotide synthesis conditions” in the context of primer hybridization refer to conditions in which a primer anneals to the nucleic acid molecule to be amplified. Exemplary nucleotide synthesis conditions are 10 mM TrisHCl pH 8.3, 1.5 mM MgCl, 50 mM KCl, 62° C. Other exemplary nucleotide synthesis conditions are 16.6 mM ammonium sulfate, 67 mM Tris pH 8.8, 6.7 mM MgCl, 10 mM 2-mercaptoethanol, 60° C. Those of skill in the art are familiar with parameters that affect hybridization; such as temperature, probe or primer length and composition, buffer composition and pH, and salt concentration can readily adjust these parameters to achieve specific hybridization of a nucleic acid to a target sequence.

As used herein, “complementary base pairs” refer to Watson-Crick base pairs (e.g., G to C and A to T in DNA and G to C and A to U in RNA) or the equivalent thereof when non-natural or atypical nucleotides are used. Two nucleic acid strands that are complementary contain complementary base pairing. A probe is not complementary when mismatches such as G-T, G-A, C-T or C-A arise when a probe or primer hybridizes to a nucleic acid target gene molecule.

As used herein “substantially complementary” refers to primers that are sufficiently complementary to hybridize with nucleic acid target gene molecules having a desired sequence under nucleic acid synthesis conditions. Primers should have sufficient complementarity to hybridize to a desired nucleic acid target gene molecule and permit amplification of the nucleic acid target gene molecule. For example, a primer used in the methods disclosed herein can be 100% complementary with the nucleic acid target gene molecule desired to be amplified. In another example, a primer can have 1, 2, 3, or more mismatches, provided that the primer can be used to amplify at least one nucleic acid target gene molecule desired to be amplified. For example, a nucleic acid target gene molecule can have three cytosine nucleotides in the region with which a primer hybridizes; when only one of the three C nucleotides are methylated, treatment with bisulfite can convert the two unmethylated C nucleotides to U nucleotides, and a primer 100% complementary to a nucleic acid target gene molecule having three C nucleotides can still hybridize to a nucleic acid target gene molecule having only one C nucleotide, such that the nucleic acid target gene molecule having only one C nucleotide can still be amplified.

As used herein “nucleic acid” refers to polynucleotides such as deoxyribonucleic acid (DNA) and ribonucleic acid (RNA). The term also includes, as equivalents, derivatives, variants and analogs of either RNA or DNA made from nucleotide analogs, single-stranded (“sense” or “antisense”, “plus” strand or “minus” strand, “forward” reading frame or “reverse” reading frame) and double-stranded polynucleotides. Deoxyribonucleotides include deoxyadenosine, deoxycytidine, deoxyguanosine and deoxythymidine. For RNA, the base cytosine is replaced with uracil.

As used herein, “mass spectrometry” encompasses any suitable mass spectrometric format known to those of skill in the art. Such formats include, but are not limited to, Matrix-Assisted Laser Desorption/Ionization, Time-of-Flight (MALDI-TOF), Electrospray (ES), IR-MALDI (see, e.g., published International PCT application No. 99/57318 and U.S. Pat. No. 5,118,937), Ion Cyclotron Resonance (ICR), Fourier Transform and combinations thereof.

As used herein, the phrase “mass spectrometric analysis” refers to the determination of the mass to charge ratio of atoms, molecules or molecule fragments.

As used herein, a “reference nucleic acid molecule” refers to a nucleic acid molecule known to be methylated or unmethylated, or a nucleic acid molecule in which the methylation state of one or more nucleotide loci of the nucleic acid molecule is known. A reference nucleic acid can be used to calculate or experimentally derive reference masses. A reference nucleic acid used to calculate reference masses is typically a nucleic acid containing a known sequence with known methylated nucleotide loci. A reference nucleic acid used to experimentally derive reference masses can have, but is not required to have, a known sequence or known methylated nucleotide loci; methods such as those disclosed herein or otherwise known in the art can be used to identify a reference nucleic acid as methylated even when the reference nucleic acid does not have a known sequence.

As used herein, a “correlation” between a nucleic acid target gene molecule and a reference, including a “correlation” between a nucleotide locus in a nucleic acid target gene molecule and a nucleotide locus in a reference, refers to a similarity or identity of the methylation state of a nucleic acid target gene molecule or nucleotide locus to that of a reference, such that the nucleic acid target gene molecule and the reference are expected to have at least one undefined locus with the same methylation state. For example, when the methylation state of fewer than all nucleotide loci of a nucleic acid target gene molecule have been identified, and when there is a correlation between a reference nucleic acid and a nucleic acid target gene, one or more of the unidentified loci of the nucleic acid target gene molecule can be expected to have the same methylation state as the corresponding nucleotide locus in the reference.

As used herein, the term “correlates” as between a specific prognosis of a sample or of an individual and the changes in methylation state of a nucleic acid target gene region refers to an identifiable connection between a particular prognosis of a sample or of an individual and its methylation state.

As used herein, “nucleic acid synthesis” refers to a chemical or biochemical reaction in which a phosphodiester bond is formed between one nucleotide and a second nucleotide or an oligonucleotide. Nucleic acid synthesis can include enzymatic reactions such as DNA replication reactions such as PCR or transcription, or chemical reactions such as solid phase synthesis. Nucleic acid synthesis conditions refers to conditions of a nucleic acid molecule-containing solution in which nucleotide phosphodiester bond formation is possible. For example, a nucleic acid target gene molecule can be contacted with a primer, and can be treated under nucleic acid synthesis reactions, which can include, for example, PCR or transcription conditions, and, when the primer hybridizes to the nucleic acid target gene molecule, nucleotides can be synthesized onto the primer, that is, nucleotides can be enzymatically added via phosphodiester linkage to the 3′ end of primer, however, when no primer is hybridized to the nucleic acid target gene molecule, it is possible that no nucleotides are synthesized onto the primer.

As used herein, “amplifying” refers to increasing the amount of a nucleic acid molecule or a number of nucleic acid molecules. Amplification may be performed by one or more cycles of polymerase chain reaction (PCR). Based on the 5′ and 3′ primers that are chosen the region or regions of the nucleic acid molecule or nucleic acid molecules to be amplified may be selected. Amplification can be by any means known to those skilled in the art, including use of the PCR, transcription, and other such methods.

As used herein, “specifically amplifying” refers to increasing the amount of a particular nucleic acid molecule based on one or more properties of the molecule. For example, a nucleic acid molecule can be specifically amplified using specific hybridization of one or more primers to one or more regions of the nucleic acid molecule in PCR. Typically, specifically amplifying includes nucleic acid synthesis of a nucleic acid target gene molecule where a primer hybridizes with complete complementarity to a nucleotide sequence in the nucleic acid target gene molecule.

As used herein a “primer” is a polynucleotide such as DNA or RNA that because of its specific nucleotide sequence is able to hybridize to a template nucleic acid, whereupon an enzyme can catalyze addition of one or more nucleotides to the 3′ hydroxyl group of the primer thorough formation of a phosphoester or phosphodiester bond in a nucleotide synthesis reaction such as transcription or DNA replication.

As used herein, a “methylation specific primer” or “methylation state specific primer” refers to a primer that can specifically hybridize with a nucleic acid target gene region or a methylation-specific reagent-treated nucleic acid target gene molecule in accordance with the methylation state of the nucleic acid target gene molecule. For example, a nucleic acid target gene molecule can be treated with a methylation-specific reagent, resulting in a change in the nucleotide sequence of the nucleic acid target gene molecule as a function of the methylation state of the nucleic acid target gene molecule; and a methylation state specific primer can specifically hybridize to the treated methylated nucleic acid target gene molecule, without hybridizing to a treated unmethylated nucleic acid target gene molecule or without hybridizing to a treated, differently methylated nucleic acid target gene molecule. In another example, a nucleic acid target gene molecule can be treated with a methylation-specific reagent, resulting in a change in the nucleotide sequence of the nucleic acid target gene molecule as a function of the methylation state of the nucleic acid target gene molecule and a methylation state specific primer can specifically hybridize to the treated unmethylated nucleic acid target gene molecule, without hybridizing to a treated methylated nucleic acid target gene molecule or without hybridizing to a treated, differently unmethylated nucleic acid target gene molecule. Methylation specific primers that hybridize to a nucleic acid target gene molecule then can serve as primers for subsequent nucleotide synthesis reactions, such as PCR.

As used herein, an “amplified product” or “amplified nucleic acid” is any product of a nucleotide synthesis reaction using a nucleic acid target gene molecule as the template. Thus, a single-stranded nucleic acid molecule complementary to the treated nucleic acid target gene molecule and formed in the first amplification step is an amplified product. In addition, products of subsequent nucleotide synthesis reactions, which contain the same sequence as the treated nucleic acid target gene molecule, or the complement thereof, are amplification products. An amplification product can be a single-stranded nucleic acid molecule or a double-stranded nucleic acid molecule.

As used herein, “fragmentation” or “cleavage” refers to a procedure or conditions in which a nucleic acid molecule, such as a nucleic acid target gene molecule or amplified product thereof, is severed into two or more smaller nucleic acid molecules. Such fragmentation or cleavage can be sequence specific, base specific, or nonspecific, and can be accomplished by any of a variety of methods, reagents or conditions, including, for example, chemical, enzymatic, physical fragmentation.

As used herein, “fragments”, “cleavage products”, “cleaved products” or grammatical variants thereof, refers to nucleic acid molecules resultant from a fragmentation or cleavage of a nucleic acid target gene molecule or amplified product thereof. While such fragments or cleaved products can refer to all nucleic acid molecules resultant from a cleavage reaction, typically such fragments or cleaved products refer only to nucleic acid molecules resultant from a fragmentation or cleavage of a nucleic acid target gene molecule or the portion of an amplified product thereof containing the corresponding nucleotide sequence of a nucleic acid target gene molecule. For example, it is within the scope of the present methods, compounds and compositions, that an amplified product can contain one or more nucleotides more than the amplified nucleotide region of the nucleic acid target gene sequence (e.g., a primer can contain “extra” nucleotides such as a transcriptional initiation sequence, in addition to nucleotides complementary to a nucleic acid target gene molecule, resulting in an amplified product containing “extra” nucleotides or nucleotides not corresponding to the amplified nucleotide region of the nucleic acid target gene molecule). In such an example, the fragments or cleaved products corresponding to the nucleotides not arising from the nucleic acid target gene molecule will typically not provide any information regarding methylation in the nucleic acid target gene molecule. One skilled in the art can therefore understand that the fragments of an amplified product used to provide methylation information in the methods provided herein are fragments containing one or more nucleotides arising from the nucleic acid target gene molecule, and not fragments containing nucleotides arising solely from a sequence other than that in the nucleic acid target gene molecule. Accordingly, one skilled in the art will understand the fragments arising from methods, compounds and compositions provided herein to include fragments arising from portions of amplified nucleic acid molecules containing, at least in part, nucleotide sequence information from or based on the representative nucleic acid target gene molecule.

As used herein, “base specific cleavage” refers to selective cleavage of a nucleic acid at the site of a particular base (e.g., A, C, U or G in RNA or A, C, T or G in DNA) or of a particular base type (e.g., purine or pyrimidine). For example, C-specific cleavage refers to cleavage of a nucleic acid at every C nucleotide in the nucleic acid.

As used herein, the phrase “non-specifically cleaved”, in the context of nucleic acid cleavage, refers to the cleavage of nucleic acid target gene molecule at random locations throughout, such that various cleaved fragments of different size and nucleotide sequence content are randomly generated. Cleavage at random locations, as used herein, does not require absolute mathematical randomness, but instead only a lack of sequence-based preference in cleavage. For example, cleavage by irradiative or shearing means can cleave DNA at nearly any position, however, such methods can result in cleavage at some locations with slightly more frequency than other locations. Nevertheless, cleavage at nearly all positions with only a slight sequence preference is still random for purposes herein. Non-specific cleavage using the methods described herein can result in the generation of overlapping nucleotide fragments.

As used herein, the phrase “statistically range in size” refers to the size range for a majority of the fragments generated using cleavage methods known in the art or disclosed herein, such that some of the fragments can be substantially smaller or larger than most of the other fragments within the particular size range. An example of such a statistical range in sizes of fragments is a Poisson distribution. For example, the statistical size range of 12-30 bases also can include some oligonucleotides as small as 1 nucleotide or as large as 300 nucleotides or more, but these particular sizes statistically occur relatively rarely. In some embodiments, there is no limit to the statistical range of fragments. In other embodiments, a statistical range of fragments can specify a range such that 10% of the fragments are within the specified size range, where 20% of the fragments are within the specified size range, where 30% of the fragments are within the specified size range, where 40% of the fragments are within the specified size range, where 50% of the fragments are within the specified size range, where 60% or more of the fragments are within the specified size range, where 70% or more of the fragments are within the specified size range, where 80% or more of the fragments are within the specified size range, where 90% or more of the fragments are within the specified size range, or where 95% or more of the fragments are within the specified size range.

As used herein, the phrase “set of mass signals” or a “mass peak pattern” refers to two or more mass determinations made for each of two or more nucleic acid fragments of a nucleic acid molecule. A “mass pattern” refers to two or more masses corresponding to two or more nucleic acid fragments of a nucleic acid molecule.

As used herein, a “subject” includes, but is not limited to, an animal, plant, bacterium, virus, parasite and any other organism or entity that has nucleic acid. Among animal subjects are mammals, including primates, such as humans. As used herein, “subject” may be used interchangeably with “patient” or “individual”.

As used herein, “normal”, when referring to a nucleic acid molecule or sample source, such as an individual or group of individuals, refers to a nucleic acid molecule or sample source that was not selected according to any particular criterion, and generally refers to a typical nucleotide sequence of a nucleic acid molecule or health condition of a sample source (e.g., one or more healthy subjects or one or more subjects that do not a disease). For example, a normal methylation state of a particular nucleotide locus can be the wild type methylation state of the nucleotide locus. In another example, a group of normal subjects can be a group of subjects not having a particular phenotype (such as a disease).

As used herein, a “phenotype” refers to a set of parameters that includes any distinguishable trait of an organism. A phenotype can be physical traits and/or mental traits, such as emotional traits. A phenotype may also include a subject's disease prognosis.

As used herein, a “methylation” or “methylation state” correlated with a disease, disease outcome or outcome of a treatment regimen refers to a methylation state of a nucleic acid target gene region or nucleotide locus that is present or absent more frequently in subjects with a known disease, disease outcome or outcome of a treatment regimen, relative to the methylation state of a nucleic acid target gene region or nucleotide locus than otherwise occur in a larger population of individuals (e.g., a population of all individuals).

As used herein, an “poor prognosis treatment regimen” refers to an AML treatment course that is likely to induce complete remission and prevent relapse, but is either experimental, difficult to administer (e.g., finding an appropriate stem cell donor), palliative in nature (e.g., treatments designed to prevent and control the side effects of cancer and its treatment or provide comfort and support for the patient until they are deceased), or any treatment that is not included herein, but a medical practitioner may deem appropriate for a patient with a poor AML prognosis. Examples of poor prognosis treatments may include, but are not limited to, administering a chemotherapy agent (e.g., a non-standard, non-aggressive or experimental chemotherapy agent), performing an allogeneic stem cell transplant, administering all-trans-retinoic acid, administering a novel therapy and combinations of the foregoing. In older and/or poor prognosis patients, the benefit of intensive therapy has been more difficult to document and therefore pursuit of novel therapies as consolidation for these patients is usually pursued. A “novel therapy” as used herein refers to an investigational treatment (e.g., monoclonal antibodies, new consolidation chemotherapy regimens, multiple drug resistance inhibitors, biological modifier therapies, and demethylating agents). An example of a demethylation agent is decitabine, which can be administered alone or in combination with other known therapeutic compounds (e.g., Ruter et al., Int. J. Hematol. 80(2):128-35 (2004)).

As used herein, a “good prognosis treatment regimen” refers to a standard AML treatment course that is likely to induce complete remission and prevent relapse or any treatment that is not included herein that a medical practitioner may deem appropriate for a patient with a good AML prognosis. Standard therapy includes a 7-day continuous infusion of cytarabine, and a 3-day course of an anthracycline. The anthracyclines include daunorubicin (Cerubidine), doxorubicin (Adriamycin, Rubex), epirubicin (Ellence, Pharmorubicin), and idarubicin (Idamycin). If patients have not achieved a remission, another induction course of treatment will be given immediately. Generally, the standard treatment regimen is intense (high dosage and high frequency). The influence of intensifying therapy with traditional chemotherapy agents such as cytarabine and anthracyclines in younger and/or good prognosis patients appears to increase the cure rate of AML. This treatment is often supplemented by performing a blood transfusion, performing a platelet transfusion, administering antibiotics and blood cell growth factors.

As used herein, a “classification algorithm” refers to a statistical procedure in which individual items are placed into groups based on quantitative information on one or more characteristics inherent in the items (referred to as traits, variables, characters, etc) and based on a training set of previously labeled items. Examples of classification algorithms include, but are not limited to, Linear classifiers (Fisher's linear discriminant, Logistic regression, Naive Bayes classifier, Perceptron), k-nearest neighbor, Boosting, Decision trees, Neural networks, Bayesian networks, Support vector machines, Hidden Markov models, Principle Component Analysis and Random Forest. Specific algorithms and packages utilized in the present invention include the “gregmisc” package, which may be used for two-dimensional clustering; the “hclust” package, which may be used for hierarchical cluster analysis; the “survival” package, which may be used for Cox regression analysis; the Kaplan Meier estimates and the “superpc” package (Bair and Tibshirani, PloS Biol 2:E108 (2004)), which may be used for supervised principle components analysis, and the pair-wise Euclidean distances and the complete linkage clustering algorithm, which may be used for two-way hierarchical cluster analysis. Any classification algorithm known by those skilled in the art may similarly be used in the present invention—either alone or in combination with those disclosed here.

As used herein, a “data processing routine” refers to a process, that can be embodied in software, that determines the biological significance of acquired data (i.e., the ultimate results of an assay or analysis). For example, the data processing routine can make a genotype determination based upon the data collected. In the systems and methods herein, the data processing routine also can control the instrument and/or the data collection routine based upon the results determined. The data processing routine and the data collection routines can be integrated and provide feedback to operate the data acquisition by the instrument, and hence provide assay-based judging methods.

As used herein, a “plurality of genes” or a “plurality of nucleic acid target gene molecules” includes at least two, five, 10, 25, 50, 100, 250, 500, 1000, 2,500, 5,000, 10,000, 100,000, 1,000,000 or more genes or nucleic acid target gene molecules. A plurality of genes or nucleic acid target gene molecules can include complete or partial genomes of an organism or even a plurality thereof. Selecting the organism type determines the genome from among which the gene or nucleic acid target gene molecules are selected.

As used herein, “sample” refers to a composition containing a material to be detected. Samples include “biological samples”, which refer to any material obtained from a living source, for example, an animal such as a human or other mammal, a plant, a bacterium, a fungus, a protist or a virus or a processed form, such as amplified or isolated material. The biological sample can be in any form, including a solid material such as a tissue, cells, a cell pellet, a cell extract, a biopsy, or feces, or a biological fluid such as urine, whole blood, plasma, serum, interstitial fluid, peritoneal fluid, lymph fluid, ascites, sweat, saliva, follicular fluid, breast milk, non-milk breast secretions, cerebral spinal fluid, seminal fluid, lung sputum, amniotic fluid, exudate from a region of infection or inflammation, a mouth wash containing buccal cells, synovial fluid, or any other fluid sample produced by the subject. In addition, the sample can be solid samples of tissues or organs, such as collected tissues, including bone marrow, epithelium, stomach, prostate, kidney, bladder, breast, colon, lung, pancreas, endometrium, neuron, muscle, and other tissues. Samples can include organs, and pathological samples such as a formalin-fixed sample embedded in paraffin. If desired, solid materials can be mixed with a fluid or purified or amplified or otherwise treated. Samples examined using the methods described herein can be treated in one or more purification steps in order to increase the purity of the desired cells or nucleic acid in the sample, Samples also can be examined using the methods described herein without any purification steps to increase the purity of desired cells or nucleic acid. In particular, herein, the samples include a mixture of matrix used for mass spectrometric analyses and a biopolymer, such as a nucleic acid.

As used herein, “array” refers to a collection of elements, such as nucleic acids. Typically an array contains three or more members. An addressable array is one in which the members of the array are identifiable, typically by position on a solid support. Hence, in general the members of the array will be immobilized to discrete identifiable loci on the surface of a solid phase. Arrays include a collection on elements on a single solid phase surface, such as a collection of nucleotides on a chip.

As use herein, the term “data set” refers to numerical values obtained from the analysis, such as by mass spectral analysis of the nucleic acid target gene region. These numerical values associated with analysis may be values such as peak height, area under the curve and molecular mass for example in the case of mass spectral analysis.

As used herein the term “data structure” refers to a combination of two or more data sets, applying one or more mathematical manipulations to one or more data sets to obtain one or more new data sets, or manipulating two or more data sets into a form that provides a visual illustration of the data in a new way. An example of a data structure prepared from manipulation of two or more data sets would be a hierarchical cluster.

The present invention also provides a method for identifying an unknown phenotype of a tissue or cell that correlates with changes in the methylation state of the tissue or cell comprising; treating a nucleic acid sample from said tissue or cell with a reagent that modifies unmethylated cytosine to produce uracil; amplifying a nucleic acid target gene region using at least one primer that hybridizes to a strand of the nucleic acid target gene region producing amplified nucleic acids; determining the characteristic methylation state of the nucleic acid target gene region by base specific cleavage and identification of methylation sites of the amplified nucleic acids; and comparing the ratio of methylated cytosine to unmethylated cytosine for each of the methylation sites of the characteristic methylation state of the sample from the tissue or cell nucleic acid to the ratio of methylated cytosine to unmethylated cytosine for each of the methylation sites of a tissue or cell nucleic acid sample of the same type having a known phenotype thereby identifying the unknown phenotype.

In one preferred aspect of the present invention analysis of the DNA methylation of a nucleic acid target gene region is obtained by MALDI-TOF MS analysis of base-specific cleavage products derived from amplified nucleic acid target gene molecules. In general, a PCR amplification product is generated from bisulfite treated DNA, which is transcribed in vitro into a single stranded RNA molecule and subsequently cleaved base-specifically by an endoribonuclease. The conversion of cytosine to uracil during bisulfite treatment generates different base specific cleavage patterns that can be readily analysed by MALDI-TOF MS. These spectral analyses may be used to determine the ratio of methylated versus non-methylated nucleotide at each methylation site of the nucleic acid target gene region. One skilled in the art will recognise that the methylation state of any nucleic acid, nucleic acid target gene region or gene of interest may be determined using the methods of the present invention. In addition, one skilled in the art would recognise the importance of the location of CpG islands in identifying novel, unique or specific methylation states for diagnostic purposes. Correspondingly, the location of a CpG island in a nucleic acid of interest may indicate other CpG islands of significance located in and around, or in close proximity to, the initially identified CpG island. Consequently it would be reasonable that one skilled in the art would look to other areas in proximity to initially identified CpG island to locate other CpG islands of interest.

### Acute Myeloid Leukemia (AML) and Sample Selection

Acute myelogenous leukemia (AML) is the most common form of leukemia with more than 10,000 people diagnosed each year, according to National Cancer Institute estimates.

### Etiology

Heredity, radiation, chemical and other occupational exposures, and drugs have been implicated in the development of AML. There is no direct evidence of a viral etiology in AML.

Heredity: Certain syndromes with somatic cell chromosome aneuploidy, e.g., Down Syndrome, are associated with an increased incidence of AML. Inherited diseases with excessive chromatin fragility, e.g., ataxia telangiectasia, are also associated with AML.

Chemical and Other Exposures: Exposure to benzene, which is used as a solvent in the chemical, plastic, rubber, and pharmaceutical industries, is associated with an increased incidence of AML. Smoking and exposure to petroleum products, paint, embalming fluids, ethylene oxide, herbicides, pesticides, and electromagnetic fields have also been associated with an increased risk of AML.

Drugs: Antineoplastic drugs are the leading cause of drug-related (or treatment-associated) AML. Alkylating agent-associated leukemia occurs on average 48-72 months after exposure and demonstrates aberrations in chromosomes 5 and 7. Topoisomerase II inhibitor-associated leukemias occur 1-3 years after exposure and usually have aberrations involving chromosome band 11 q23. Similarly, chloramphenicol, phenylbutazone, and less commonly chloroquine and methoxypsoralen have been reported to result in bone marrow failure that may evolve into AML.

### Classification

Currently, the categorization of acute leukemia into biologically distinct groups is based on morphology, cytochemistry and immunophenotype as well as cytogenetic and molecular techniques. See Table 1 below:

TABLE 1
French-American-British (FAB) Classification of AML

| | | | Cytochemistry | | | |
|---|---|---|---|---|---|---|
| | | | Peroxidase/ | | | |
| | | | Sudan | Nonspecific | | |
| FAB subtype | % of | Morphology | Black | Esterase | Flow | Cytogenetic |
| M0: Minimally | 2-3 | Immature morphology | − | − | CD13 or | |
| differentiated | | | | | 33 | |
| leukemia | | | | | | |
| M1: Myeloblastic | 20 | Few blasts with | 3% or | − | CD13, 33, | |
| leukemia without | | azurophilic granules, | more | | 34, HLA- | |
| maturation | | Auer rods, or both | | | DR+ | |
| M2: Myeloblastic | 25-30 | Azurophilic granules, | + | − | CD13, | T(8; 21) |
| leukemia with | | Auer rods are often | | | 15, 33, 34, | (q22; q22)^{e} |
| maturation | | present | | | HLA-DR+ | |
| M3: Hypergranular | 8-15 | Hypergranular | + | − | CD13, 15, | T(15; 17) |
| promyelocytic | | promyelocytes with | | | 33, HLA- | (q22; q11-12) |
| leukemia | | multiple Auer rods; | | | DR− | |
| | | Variant: hypogranular | | | | |
| M4: Myelomonocytic | 20-25 | Granulocytic and | +/− | + | CD11b, | M4Eo: inv(16 |
| leukemia | | monocytic blasts; | | | 13, 14f, | (p13q22) |
| | | Variant: M4Eo: | | | 15, 33, | |
| | | increase in abnormal | | | HLA-DR+ | |
| | | marrow eosinophils | | | | |
| M5: Monocytic | 20-25 | M5a undifferentiated; | − | + | CD11b, | 11q23 |
| leukemia | | M5b differentiated | | | 13, 14f, | translocation |
| | | | | | 15, 33, | |
| | | | | | HLA-DR+ | |
| M6: Erythroleukemia | 5 | Erythroblasts >50% of | +/− | − | CD33, | |
| (Di Gugielmo's | | nucleated cells, | | | HLA-DR+ | |
| disease | | myeloblasts >30% of | | | | |
| | | nonerythroid cells | | | | |
| M7: | 1-2 | Megakaryoblasts | − | − | CD33 | |
| Megakaryoblastic | | >30% of all nucleated | | | | |
| leukemia | | cells | | | | |

Source: BD Cheson et al, J Clin Oncol 8: 813, 1990.

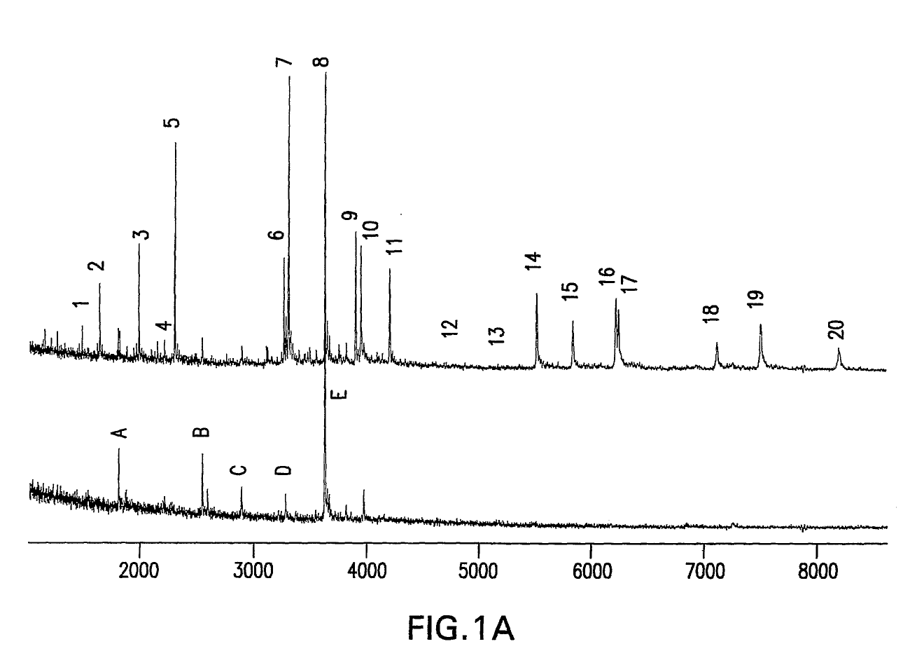

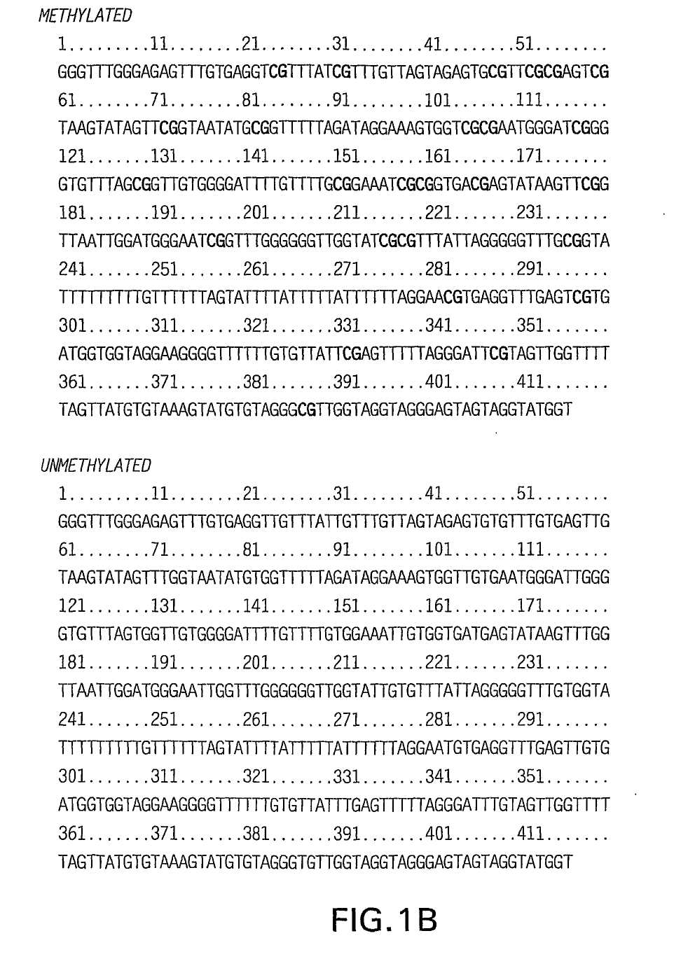

Morphologic and Cytochemical Classification: The diagnosis of AML is established by the presence of at least 20% myeloblasts in blood and/or bone marrow according to the World Health Organization classification. Once diagnosed, AML is classified based on morphology and cytochemistry according the FAB schema (see FIG. 1), which includes eight major subtypes, M0-M7.

Immunophenotypic Classification: The phenotype of human myeloid leukemia cells can be studied by multiparameter flow cytometry following labeling with monoclonal antibodies to cell-surface antigens. While results are useful for both diagnosis and prognosis, the process is complicated, time consuming and expensive. For example, M7 can often be diagnosed only by expression of the platelet-specific antigen cluster designation (CD) 41 or by electron-microscopic demonstration of myeloperoxidase.

Chromosomal Classification: Chromosomal analysis of the leukemic cell currently provides the most important pretreatment prognostic information for AML, but suffers from resolution limitations especially among those AML patients that fall into an “intermediate” risk group. Therefore, any improvement of existing AML classification methods (in terms of accuracy, speed and cost) has tremendous utility within the AML diagnostic, prognostic and therapeutic area. Two cytogenetic abnormalities have been invariably associated with a specific FAB group: T(15;17)(q22;q12) with M3 and inv(16)(p13q22) with M4Eo, and many chromosomal abnormalities have been associated primarily with one FAB group, including t(8;21)(q22;q22) with M2. Many of the recurring chromosomal abnormalities in AML have been associated with specific clinical characteristics. Changes in chromosomes in leukemia cells can be identified in 80% of children with AML. More commonly associated with younger age onset are t(8;21) and t(15;17), and with older age onset, del(5q) and del(7q). With currently available treatments, 30-50% of children with AML are cured. It is important to identify those children who can be cured with standard treatments and those who should receive more individualized treatment or more aggressive treatment. The distinct type of chromosomal abnormality present at diagnosis has been shown to help identify patients with a “good” or “bad” outcome.

For example, in one Pediatric Oncology Group study, outcomes of 478 children with AML were reported. They found that children with an inverted 16th chromosome had a survival rate without relapse of 58%, those with a translocation of chromosomes 8 and 21 had a survival rate without relapse of 45% and patients with no chromosomal abnormalities had a survival rate without relapse of 45%. Children with translocation of chromosomes 15 and 17 had a survival rate without relapse of 20% and children with 11q23 abnormalities had a survival rate of 24%. This study demonstrates the benefit of using clinical data to decide which treatment regimen is best suited for patients suffering from AML.

Molecular Classification: Molecular studies of many recurring cytogenetic abnormalities have revealed genes that may by involved in leukogenesis. The 15;17 translocation encodes a chimeric protein, Pm1/Rarα, which is formed by the fusion of the retinoic acid receptor-α (RARα) gene from chromosome 17 and the promyelocytic leukemia (PML) gene from chromosome 15. The Pm1-Rarα fusion protein tends to suppress gene transcription and blocks differentiation of the cells. Pharmacologic doses of the Rarα ligand, all-trans-retinoic acid (tretinoin), relieve the block and promote differentiation.

Similar translocations resulting in molecular aberrations involved in leukogenesis include inv(16), t(8;21), and 11q23, all of which are increasingly being used for diagnosis and detection of residual disease after treatment. Molecular aberrations are also being identified that are useful for classifying risk of relapse in patients without cytogenetic abnormalities. A partial tandem duplication (PTD) of the MLL gene is found in 5-10% of patients with normal cytogenetics and results in short remission duration.

Recently, more wide-scale gene expression profiling has been used in to improve the molecular AML classification. Initial studies have provided useful results identifying novel AML subgroups and prognostic gene expression signatures (Bullinger L. et al. N Engl J Med 350:1605-16 (2004)) and (Valk P J et al. N Engl J Med 350:1617-28 (2004)). In addition, Bullinger et a observed differential expression of DNA methylation enzymes (regulators) DNMT3A and DNMT3B in AML patients. DNA methylation is recognized as a key regulatory element of gene expression (Feinberg, A P Nat Genet. 27:9-10 (2001), therefore these findings point to a potential pathogenic role of aberrant DNA methylation patterns in subgroups of AML patients resulting in distinct gene expression signatures. In particular, aberrant promoter hypermethylation represents an important mechanism in the initiation and progression of human cancer. Aberrant methylation patterns have also been described in AML by Toyota, M. et al (Blood 97:2823-9 (2001)) and Issa JP (Nat Rev Cancer 4:988-93 (2004)).

Thus, in an embodiment of the invention, the methods described herein may be used alone or in combination with currently used morphology (e.g., the percent of myeloblasts in blood and/or bone marrow), cytochemistry, immunophenotype (e.g., platelet-specific antigen cluster designation) as well as cytogenetic and molecular techniques (e.g., gene expression) to provide a better means to stratify AML patients into different risk groups and accordingly administer the proper treatment regimen as determined by one skilled in the art.

### Clinical Presentation

Symptoms: Patients with AML most often present with nonspecific symptoms that begin gradually or abruptly and are the consequence of anemia, leukocytosis, leukopenia or leukocyte dysfunction, or thrombocytopenia. Nearly half have had symptoms for greater than three months before the leukemia was diagnosed.

Half of leukemia patients mention fatigue as the first symptom, but most complain of fatigue or weakness at the time of first diagnosis. Anorexia and weight loss are common. Fever with or without an identifiable infection is the initial symptom in 10% of patients. Signs of abnormal hemostasis are noted in 5% of patients. On occasion, bone pain, lymphaderiopathy, non-specific cough, headache, or diaphoresis is the presenting symptom.

Physical Findings: Fever, splenomegaly, hepatomegaly, lymphadenopathy, sternal tenderness, and evidence of infection and hemorrhage are often found at diagnosis. Significant gastrointestinal bleeding, intrapulmonary hemorrhage, or intracranial hemorrhage occur most often in acute promyelocytic leukemia (APL). Retinal hemorrhages are detected in 15% of patients.

Hematologic Findings: Anemia is usually present at diagnosis and can be severe. The degree varies considerably irrespective of other hematologic findings, splenomegaly, or the duration of symptoms. Decreased erythropoiesis often results in a reduced reticulocyte count, and erythrocyte survival is decreased by accelerated destruction. Active blood loss also contributes to the anemia.

The median presenting leukocyte count is about 15,000/μl. Between 25 and 40% of patients have counts<5,000/μl, and 20% have counts>100,000/μl. Fewer than 5% have no detectable leukemic cells in the blood. Poor neutrophil function may be noted functionally by impaired phagocytosis and migration and morphologically by abnormal lobulation and deficient granulation.

Platelet counts<100,000/μl are found at diagnosis in 75% of patients, and about 25% have counts<25,000/μl.

Pretreatment Evaluation: Once the diagnosis of AML is suspected, a rapid evaluation and initiation of appropriate therapy should follow. Factors that have prognostic significance, for example, for achieving complete remission (CR), for predicting the duration of CR or for predicting survivability, should also be assessed before initiating treatment.

Prognostic Factors

Although 70-80% of younger AML patients achieve complete remission (CR) with current chemotherapy induction regimens, more than half of these patients relapse and die of their disease. More intensive consolidation treatments, such as allogeneic stem cell transplantation, often prevent relapse, but are themselves associated with high treatment-related mortality (Giles, F. J. et al. Acute myeloid leukemia. Hematology (Am Soc Hematol Educ Program), 73-110 (2002)). Therefore, it is crucial to stratify patients by risk in order to prescribe the appropriate treatment regimen that matches their risk profile. For example, a patient with a poor prognosis (i.e., high risk) may be more willing to assume the risks associated with intensive consolidation treatments, such as allogeneic stem cell transplantation.

Many factors influence the likelihood of entering CR, the length of CR, and the curability of AML. In an embodiment of the invention, the methylation-based prognostic methods provided herein may be used to predict the probability of a subject's likelihood of complete remission following induction therapy wherein said likelihood of complete remission is correlated with changes in the methylation state of said subject. CR is defined after examination of both blood and bone marrow. The blood neutrophil count must be >1500/μl and the platelet count>100,000/μl. Hemoglobin concentration or hematocrit are not considered in determining CR. Circulating blasts should be absent. While rare blasts may be detected in the blood during marrow regeneration, they should disappear on successive studies. Bone marrow cellularity should be >20% with trilineage maturation. The bone marrow should contain <5% blasts, and Auer rods should be absent. For patients in CR, reverse transcriptase PCR to detect AML-associated molecular abnormalities and FISH to detect AML-associated cytogenetic aberrations are currently used to detect residual disease. Methods to detect minimal residual disease may become a reliable discriminator between patients in CR who do or do not require additional and/or alternative therapies. Prognostic factors are influenced by the treatment used.

Other prognostic factors include the following: age at diagnosis, chromosome findings at diagnosis, history of an antecedent hematologic disorder, history of a previous malignany, a high presenting leukocyte count, and other factors described in the FAB classification diagnosis of Table 1 (e.g., leukemic cell characteristics such as ultrastructural features, immunophenotype, expression of the MDR1 gene, etc.). In addition to pretreatment variables, several treatment factors correlate with prognosis in AML, including the quickness with which the blast cells disappear from the blood after the institution of therapy. In addition, patients who achieve CR after one induction cycle have longer CR durations than those requiring multiple cycles.

### Treatment Options for AML

Although treatment of acute myeloid leukemia (AML) has improved dramatically over the past 30 years, the majority of patients with this disease will die within two years of diagnosis. Researchers have learned that the best way to cure patients with AML is to administer large doses of chemotherapeutic agents in a short period of time. The concept is to kill leukemia cells within 6 months before resistance to the drugs occurs. Therapy is divided into two phases: remission induction and post-remission consolidation/maintenance. Induction chemotherapy is administered to produce a complete remission (CR) in the bone marrow. Once CR is obtained, further therapy must be used to prolong survival and achieve cure. The initial induction treatment and subsequent consolidation therapy are often chosen based upon the prognostic factors described above. In an embodiment of the invention, the initial induction treatment may be chosen based soley upon the methylation-based prognostic methods provided herein or in combination with existing prognostic factors or markers. The influence of intensifying therapy with traditional chemotherapy agents such as cytarabine and anthracyclines in younger and/or lower risk patients appears to increase the cure rate of AML. In older and/or higher risk patients, the benefit of intensive therapy has been more difficult to document and therefore pursuit of novel therapies as consolidation for these patients is being actively pursued.

Remission Induction Therapy: During remission induction therapy, patients are given large doses of chemotherapy over a period of 5-7 days. These chemotherapy drugs kill leukemia cells and normal bone marrow cells. The major side effects of these drugs are related to toxicities of rapidly growing cells in the body, i.e., normal bone marrow, skin and the gastrointestinal tract. Each drug also has specific side effects for other organs.

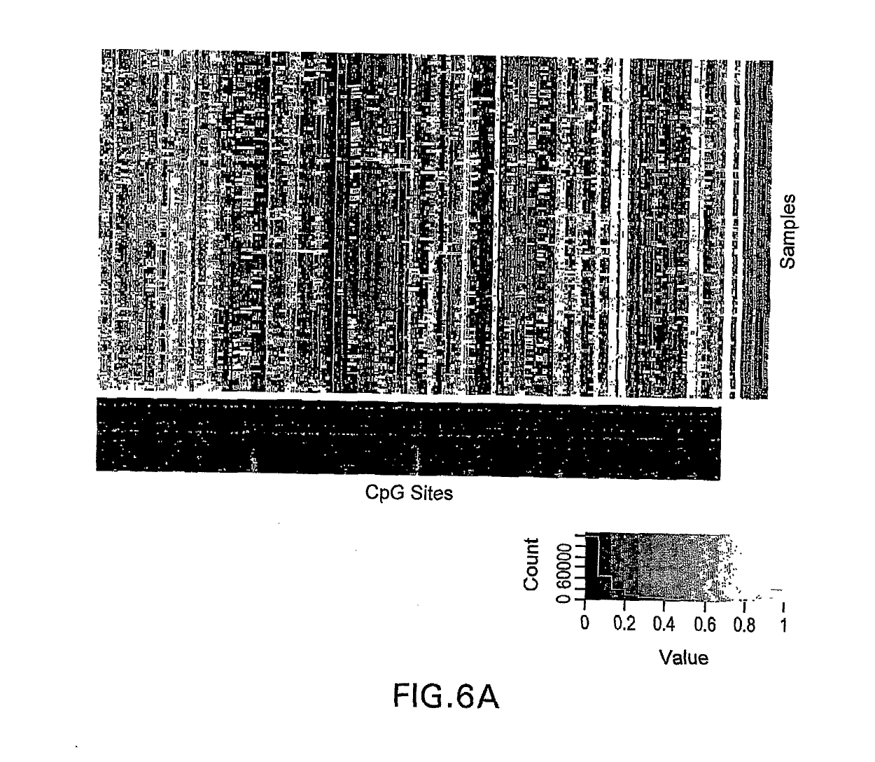

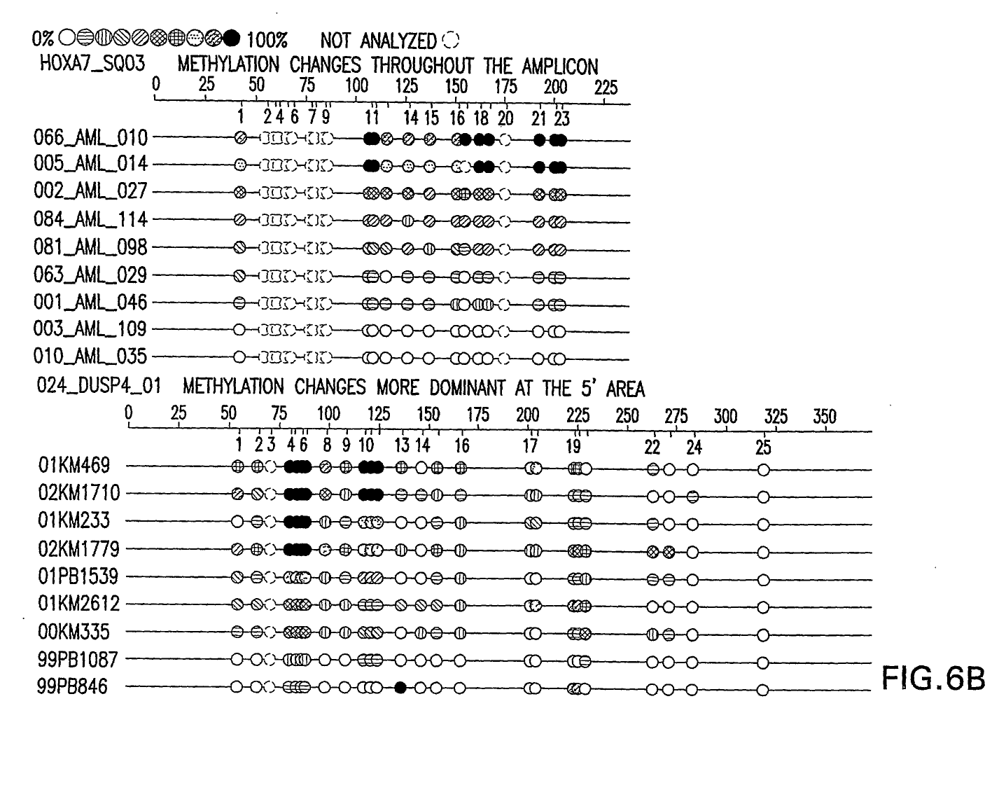

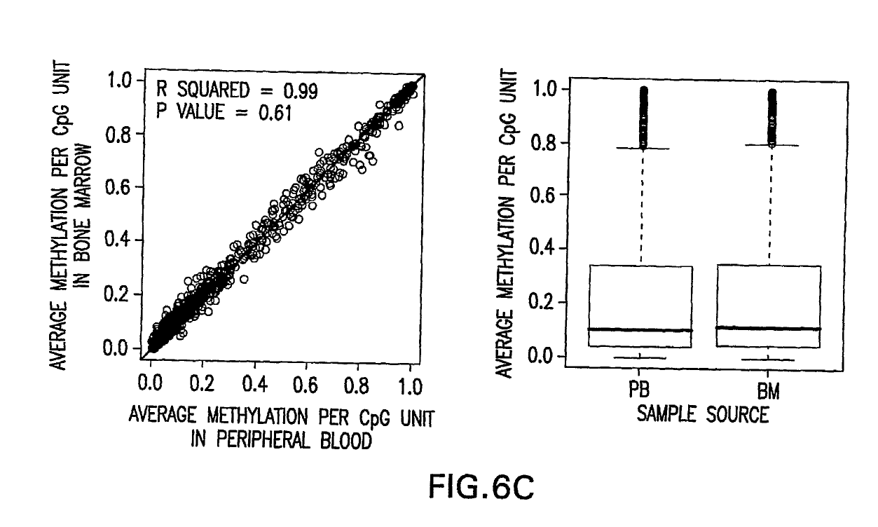

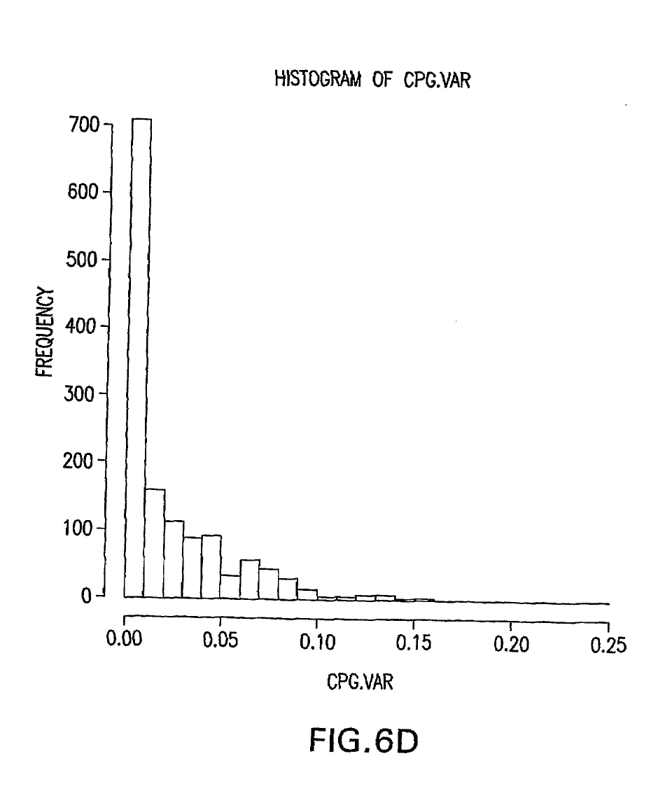

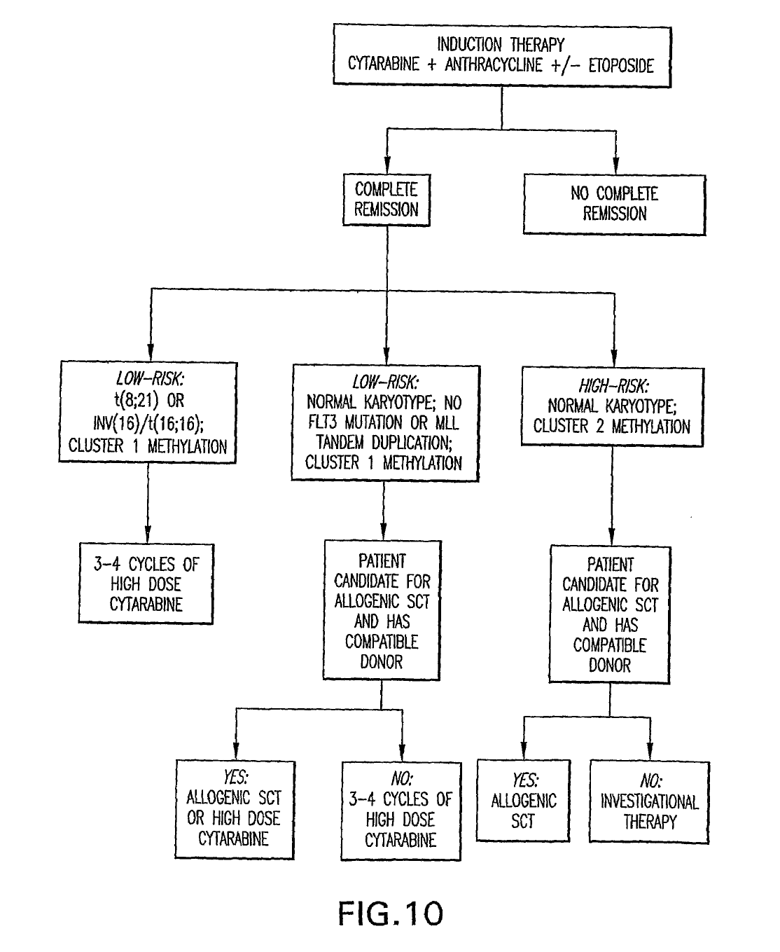

FIG. 10 is a flow chart outlining the therapeutic options available to a newly diagnosed AML patient. In FIG. 6A, the factors determining a low-risk vs a high-risk patient may be supplemented by the methylation-based prognostic methods provided herein. For all forms of AML, except APL, standard therapy includes a 7-day continuous infusion of cytarabine, and a 3-day course of an anthracycline. The anthracyclines include daunorubicin (Cerubidine), doxorubicin (Adriamycin, Rubex), epirubicin (Ellence, Pharmorubicin), and idarubicin (Idamycin). Following induction, patients typically require 2-3 weeks for bone marrow blood cell production to recover. During this time, patients often require blood and platelet transfusions to maintain red blood cell and platelet levels. In order to reduce the risk of infection, antibiotics and blood cell growth factors that stimulate the bone marrow to produce normal white blood cells are often given during this period of time. Neupogen® and Leukine® are white blood cell growth factors currently approved by the Food and Drug Administration to facilitate white blood cell production. After 2-3 weeks, blood counts will begin to recover and often return to normal. A bone marrow examination is repeated to see if a remission has been achieved. For patients in remission, the consolidation therapy will begin. If patients have not achieved a remission, another induction course of treatment will be given immediately. However, for patients with an HLA-compatible marrow donor, consideration should be given to having an immediate allogeneic stem cell transplant without receiving a second course of induction therapy. This will depend on chances of achieving a remission with a second cycle of chemotherapy. However, even if a remission is achieved with a second cycle of chemotherapy, remission duration is often very short despite consolidation.

For patients with acute promyelocytic leukemia (M3), all-trans-retinoic acid, Vesanoid®, may be included in the remission induction regimen. Patients with acute promyelocytic leukemia typically receive Vesanoid® at some time during their treatment course. There are ongoing clinical trials to determine the optimal time to administer this drug.

Strategies to Improve Remission Induction

New Drug Development: All new drugs for the treatment of patients with AML are tested first in patients with relapsed or refractory disease. When they are found to be effective, they are then evaluated in remission induction regimens.

Mylotarg®: Mylotarg® is a targeted chemotherapy, comprised of a monoclonal antibody attached to calicheamicin, an antibiotic that kills cancer cells. Monoclonal antibodies are proteins that can be produced in a laboratory and are able to identify specific antigens (small carbohydrates and/or proteins) on the surface of certain cells and bind to them. This binding stimulates the immune system to attack and kill the cells to which the monoclonal antibody is bound. Mylotarg® is targeted against the CD 33 antigen, a protein found on the surface of cancerous blood cells. Calicheamicin is an antibiotic substance that is toxic to cancer cells. Once the monoclonal antibody binds to the cancer cells, calicheamicin is absorbed into the cells and kills them. A significant benefit of this approach is that Mylotarg® mainly targets cancer cells, thereby sparing healthy cells from destruction. This is in contrast to chemotherapy or radiation, which do not differentiate between cancer cells or healthy cells in the body, a characteristic that leads to potentially intolerable side effects.

The European Organization for Research and Treatment of Cancer (EORTC) is currently conducting a clinical trial evaluating Mylotarg® plus intensive chemotherapy consisting of mitoxantrone, cytarabine and etoposide (MICE) as induction therapy for AML patients over the age of 60. Of the 34 patients in this trial so far, nearly 50% achieved an anti-cancer response to Mylotarg® alone. Approximately two months following Mylotarg® plus chemotherapy, over 40% of patients in the trial were in a complete remission (disappearance of cancer). At four and six months following therapy, the estimated survival rates are 65% and 57%, respectively. All patients had low blood cell levels from treatment, with other side effects being consistent with standard intensive chemotherapy regimens. Other clinical trials are ongoing to evaluate Mylotarg® either alone or in combination with other therapies.

Multiple Drug Resistance Inhibitors: Patients with AML may fail to achieve a remission or relapse because of chemotherapy drug resistance genes that can be present at the time of diagnosis or are induced by treatment. Several drugs are being tested to determine if they will overcome or prevent the development of multiple drug resistance in AML as part of remission induction strategies.

Post-Remission Therapy for Acute Myeloid Leukemia

If a complete remission is achieved and no further therapy given, over 90% of patients will have a recurrence of disease in weeks to months. Therefore, patients who achieve complete remission almost always undergo some form of consolidation therapy, including sequential courses of high dose cytarabine, high-dose combination therapy with allogeneic stem cell transplant (SCT), or novel therapies, based on their predicted risk of relapse (i.e., risk-stratified therapy), their perceptions of the outcomes associated with each treatment, the availability of an HLA-matched sibling stem cell donor, their physician's bias concerning the appropriateness of each treatment option, and the geographic availability of each treatment. In an embodiment of the invention, the consolidation therapy may be chosen based soley upon the methylation-based prognostic methods provided herein or in combination with existing factors or markers provided above.

Post-remission therapy treatments are given as close together as possible. The more intensive the chemotherapy and the closer together the courses of therapy are given, the less chance the leukemia has of returning (i.e., lower doses of drugs do not work as well as higher doses of drugs). In two randomized studies, high-dose cytarabine with an anthracycline produced CR rates similar to those achieved with standard 7 and 3 regimens. However, the CR duration was longer after high-dose cytarabine than after standard-dose cytarabine.

Risks and Benefits of an Allogeneic Stem Cell Transplant: If an allogeneic stem cell transplant is performed as consolidation, patients may proceed directly to the transplant following remission induction, as there does not appear to be an advantage to receiving chemotherapy in addition to that related to the transplant itself. In essence, the transplant is the consolidation treatment. Additional chemotherapy not related to the transplant procedure for consolidation before the allogeneic transplant may increase toxicity without preventing relapses.

Patients with a suitable stem cell donor who should consider an allogeneic transplant as consolidation immediately after remission induction include patients with normal cytogenetics or adverse cytogenetic abnormalities, patients who require more than one induction cycle to achieve a remission, and patients who refuse to undergo the 3-4 cycles of consolidation and maintenance required for adequate control of disease with conventional chemotherapy alone. In an embodiment of the invention, patients with a suitable stem cell donor who should consider an allogeneic transplant as consolidation immediately after remission induction may further include patients with a poor prognosis based soley upon the methylation-based prognostic methods provided herein or in combination with existing factors or markers provided above.

Some patients with a suitable stem cell donor may consider delaying allogeneic transplant until first relapse. Patients over the age of 50-60, depending on other risk factors and general condition, patients with acute promyelocytic leukemia, and patients with “good” cytogenetic abnormalities (t8-22 and inverted 16) who can tolerate all prescribed consolidation therapy may not need to expose themselves to the immediate risk of an allogeneic stem cell transplant. In an embodiment of the invention, patients with a good prognosis based on the methylation-based methods provided herein, may not choose to undergo allogeneic transplant or may consider delaying allogeneic transplant until first relapse in order to not expose themselves to the immediate risk of an allogeneic stem cell transplant.

For patients who choose to have a stem cell transplant only if they relapse, it is important that it be performed at the very first sign of relapse. This requires bone marrow examinations every 4-6 weeks for the first 2 years after diagnosis. This strategy offers the best chance to catch the leukemia early when treatment will be more effective.

Consolidation Chemotherapy: Consolidation chemotherapy typically consists of 3 to 4 cycles of cytarabine given in high doses over 5 days in conjunction with additional chemotherapy drugs such as etoposide, daunomycin or idarubicin. Remission duration has been correlated with the dose of cytarabine and the number of cycles administered. In general, the more intensive the consolidation, the higher the cure rate.

The administration of consolidation chemotherapy interferes with the production of blood cells by the bone marrow, resulting in low white cell counts in the blood. There is usually a delay of one to two weeks after the administration of chemotherapy before the bone marrow resumes function, leaving patients with low blood counts for days or weeks. During this time, patients are often hospitalized and given antibiotics and observed for infections. Neupogen® and Leukine® are growth factors that hasten the recovery of white blood cells after the administration of chemotherapy.

Consolidation chemotherapy is typically associated with 14-21 days of myelosuppression similar to induction for each of 3-4 courses. For patients who are unwilling or unable to undergo the complex and intensive chemotherapy required for consolidation therapy, either an autologous or allogeneic transplant may be considered, since these treatments condense the therapy and produce results that are equivalent or superior to the best chemotherapy regimens.

Strategies to Improve Post-Remission Therapy

Allogeneic SCT in first CR should be strongly considered by patients with high-risk karyotypes. Patients with normal karyotypes who have other poor risk factors (antecedent hematologic disorder, failure to attain remission with a single induction course, hyperleukocytosis, PTD or the MLL gene, and FLT3 abnormalities) are also potential candidates. If a suitable HLA donor does not exist, autologous SCT or novel therapeutic approaches are considered. In each of the above cases, a patient's methylation state as determined by the methods provided herein offers the patient and doctor additional information to consider while deciding whether to pursue allogeneic SCT or any other AML treatment available.

Possible Future Treatments

While significant progress has been made in the treatment of leukemia, many patients still succumb to leukemia and the complications of treatment and better treatment strategies are still needed. Future progress in the treatment of leukemia will result from continued participation in appropriate clinical studies. Currently, there are several areas of active exploration aimed at improving the treatment of leukemia.

Monoclonal Antibodies: Another approach is to deliver additional treatment directed specifically to cancer cells and avoid harming the normal cells. Monoclonal antibodies are proteins that can be produced in a laboratory that can locate cancer cells and kill them directly or stimulate the immune system to kill them. Some monoclonal antibodies have to be linked to a radioactive isotope or a toxin in order to kill cells and the antibodies essentially serve as a delivery system. Monoclonal antibodies such as Mylotarg® can be administered alone or with chemotherapy and are being evaluated to determine whether they can improve cure rates.

Mylotarg® is the first antibody-targeted chemotherapy and represents a breakthrough technology in the treatment of AML. It is currently approved by the FDA for the treatment of elderly patients with recurrent AML and is in clinical trials to evaluate its efficacy alone and in combination with other therapies in different stages of AML. Mylotarg® is comprised of a monoclonal antibody attached to calicheamicin, an antibiotic that kills cancer cells. Mylotarg® is targeted against the CD 33 antigen, a protein found on the surface of cancerous blood cells. Calicheamicin is an antibiotic substance that is toxic to cancer cells. Once the monoclonal antibody binds to the cancer cells, calicheamicin is absorbed into the cells and kills them.

Researchers from Saint Louis University Health Sciences recently conducted a small trial to evaluate the effectiveness of Mylotarg® as consolidation therapy for patients with AML in first remission (disappearance of cancer). In this trial, five patients received Mylotarg® within one to four months of being in complete remission following standard induction and consolidation therapy. Four patients remained in complete remission for 10 to 15 months. Two of these patients later received an allogeneic stem cell transplant and are free of cancer at nine months after the transplant. All patients had severely low levels of white blood cells following treatment with Mylotarg®; however, there were no treatment-related deaths. Future clinical trials will be evaluating the effectiveness of incorporating Mylotarg® into consolidation therapy for AML.

Supportive Care: Supportive care refers to treatments designed to prevent and control the side effects of cancer and its treatment. Side effects not only cause patients discomfort, but also may prevent the optimal delivery of therapy at its planned dose and schedule. In order to achieve optimal outcomes from treatment and improve quality of life, it is imperative that side effects resulting from cancer and its treatment are appropriately managed.

Stem Cell Transplant: High-dose chemotherapy and autologous or allogeneic stem cell transplantation is currently a superior consolidation treatment option for many patients.

New Consolidation Chemotherapy Regimens: Development of new multi-drug chemotherapy treatment regimens that incorporate new or additional anti-cancer therapies for use as treatment is an active area of clinical research. New anti-cancer therapies that are being evaluated in combination with consolidation chemotherapy include the following:

Multiple Drug Resistance Inhibitors: Patients with AML fail to achieve a remission or relapse because of chemotherapy drug resistance that can be present at the time of diagnosis or are induced by treatment. Several drugs are being tested to determine if they will overcome or prevent the development of multiple drug resistance in AML as part of remission induction strategies.

Biological Modifier Therapy: Biologic response modifiers are naturally occurring or synthesized substances that direct, facilitate or enhance the body's normal immune defenses. Biologic response modifiers include interferons, interleukins and monoclonal antibodies. In an attempt to improve survival rates, these and other agents are being tested alone or in combination with chemotherapy in clinical studies. Interleukin-2 is currently being evaluated as a maintenance agent after consolidation therapy. Newer biologic agents are in the developmental phase.

Treatment for Minimal Residual Disease: Following post-remission treatment, patients typically achieve a complete remission (complete disappearance of the cancer). Unfortunately, many patients in remission still experience a relapse of leukemia. This is because not all the leukemia cells were destroyed. Doctors refer to this as a state of “minimal residual disease.” Many doctors believe that applying additional treatments when only a few leukemia cells remain represents the best opportunity to prevent the leukemia from returning. Immunotherapy to activate the body's anti-cancer defense system or other agents including monoclonal antibodies, biologic response modifiers and chemotherapy drugs can be administered over several weeks to months in an attempt to eliminate any leukemia cells remaining in the body.

### Relapsed Acute Myeloid Leukemia

If a remission is not achieved or a recurrence occurs, there are essentially two choices of therapy. Since subsequent treatment with chemotherapy is rarely curative, a palliative approach can be adopted where biologic agents, such as Mylotarg®, or chemotherapy drugs are administered in non-toxic doses to keep the disease under control for as long as possible. In this situation, the emphasis is on the quality of life and supportive care measures.

The alternative approach is to receive more intensive treatment in an attempt to produce a complete remission. There are two main intensive strategies available. For younger patients, a bone marrow or blood stem cell transplant offers a possibility for control or cure of the leukemia. The other approach is to participate in clinical trials evaluating new treatments.

The most important factors predicting response at relapse are the length of the previous CR, whether initial CR was achieved with one or two courses of chemotherapy, and the type of post-remission therapy. When predicting response at relapse, a patient's methylation state as determined by the methods provided herein offers the patient and doctor additional information to consider while deciding which post-remission therapy to select.

### Identifying Nucleic Acid Target Gene Regions

Selecting nucleic acid target gene regions of interest that harbor potential methylated sites may be based on a variety of characteristics known or available to those skilled in the art regarding the target gene of interest. Selection criteria may include for example the gene's physiological role or function in a biological pathway related to the disease/phenotype of interest, existence of mutations effecting disease/phenotype or sequence polymorphisms conferring predisposition to disease/phenotype of interest. Selection may also be based on known expression status or sequence motifs binding specific proteins relevant to methylation of gene regions/chromosomal regions. One skilled in the art would recognize that a considerable amount of information may be obtained through publication of data and experiments that may provide key indications that the methylation state of a particular gene may be of importance for future prognostic or diagnostic purposes that are the subject of the present invention.

Any type of disease condition that can be correlated with changes in the methylation state of a sample organism, tissue or cell can be analyzed with the methods of the present invention, some of these disease conditions include for example, cancer, cardiovascular disease (CVD), central nervous system disease (CNS), metabolic disease, inflammation, aging, morbidity, osteoarthritis, infection and drug response. Of particular interest are hematologic cancers, and include for example, acute myeloid leukemia and chronic myeloid leukemia.

Any nucleic acid, nucleic acid target gene region or gene may be have a potentially significant characteristic methylation state for diagnostic purposes. Consequently, any nucleic acid of interest may be analyzed using the method described herein, some examples of particular genes of interest include, APOB, APOC1, AQP1, AZGP1, BAI2, BCL11A, CD3D, CDH5, CDX2, CEACAM6, CEBPA, CKMT1, COL1A1, CTNNAL1, D2S448, DLK1, DMPK, DPEP2, DUSP4, EDG1, EMR1, EVI1, FARP1, FGFR1, FHL2, FLJ21820, FLJ23058, FLT3, FN14, FOXO1A, GAGED2, GLUL, GNG2, GS3955, GUCY1A3, GYPC, HOXA10, HOXB5, ID3, IL6ST, IL6ST, ISG20, KIAA1447, LCN2, LOC55971, LOC57228, LRP6, MAGEA3, MAP7, MEIS1, MGC14376, MGC16121, MGP, MSLN, N33, NBL1, NFKB1, NR2F2, NRP1, PBX3, PHEMX, PIK3R4, PITX2, PLCG1, PLEKHC1, PRAME, PRG2, PRO2730, PSCB5, PVALB, RARB, RBP1, RGS16, RIS1, S100P, SCAP2, SDK2, SDS.RS1, SELENBP1, SEMA3F, SERPINA3, SFTPB, SLC7A5, SLC7A7, SMG1, SNX9, SOCS1, SPI1, SPUVE, STX1A, TACSTD2, TBXAS1, TCF4, TM4SF2, TNFRSF12A, TRIB2, TUBB, TUCAN, UGCG, UGCGL2, URB and ZD52F10. Each gene may have particular regions of interest selected by a variety of methods including for example the presence of CpG islands. Particular regions of interest in the above listed genes include for example the following genome locations, chr2:21241007-21241697, chr19:50103362-50104640, chr7:30724592-30725020, chr7:99206405-99207102, chr1:31730622-31732925, chr2:60755355-60757018, chr11:117767618-117768220, chr16:64970452-64970801, chr13:27438257-27441645, chr19:46951004-46951263, chr19:38483802-38486884, chr15:41701703-41702713, chr17:45631877-45634007, chr9:107154681-107155972, chr2:3008682-3010486, chr14:100262505-100263352, chr19:50962440-50967107, chr16:66584476-66584997, chr8:29227378-29231959, chr1:101165170-101165868, chr19:6773069-6773804, chr3:170346630-170347248, chr13:96492201-96494442, chr3:13565216-13566208, chr2: 105636080-105637484, chr2:21006878-21007646, chr17:77045096-77045732, chr13:26472029-26473370, chr16:3010097-3011306, chr13:39036302-39039950, chrX:52428784-52429211, chr1:179091355-179093220, chr14:51396700-51504379, chr2:12878166-12880958, chr4:157165726-157167119, chr2:127506767-127507640, chr7:26954490-26956868, chr17:471-44962-471-46296, chr1:23354959-23355887, chr5:55306022-55307474, chr5:55306022-55307474, chr15:86893946-86894920, chr17:80127851-80129454, chr9:126265497-126267389, chr7:97641296-97642019, chr12:499-49473-49950878, chr12:12310747-12312008, chrX:151537746-151538037, chr6:136851198-136852915, chr2:66572694-66574989, chr17:1565955-1566812, chrX:133405569-133406409, chr12:14887647-14888003, chr16:737974-738711, chr8:15442008-15442658, chr1:19439532-19441598, chr4:103880296-103881832, chr15:94602978-94607689, chr10:33626928-33630403, chr9:123884168-123886915, chr11:2246681-2249508, chr3:131786186-131786806, chr4:111899467-111902268, chr20:40450460-40452461, chr14:52486659-52488289, chr22:21225941-21226252, chr11:56950917-56951226, chr3:52287110-52288097, chr19:37763716-37764648, chr22:34695834-34697316, chr3:25444558-25614624, chr3:140740648-140741626, chr1:179812558-179813341, chr3:45226871-45228831, chr4:6740035-6741149, chr7:26645943-26647225, chr17:68943217-68943473, chr12:112207972-112209000, chr1:148158675-148159233, chr3:50150785-50152342, chr14:94147980-94160642, chr2:85954841-85956938, chr16:86459756-86461161, chr14:22361530-22362118, chr16:18903385-18904879, chr6:158152689-158154521, chr16:11255843-11258504, chr11:47356165-47356782, chr11:86237294-86238397, chr7:72545287-72546501, chr2:47507930-47508907, chr7:138884485-138885973, chr18:51595863-51597029, chrX:37451260-37452579, chr16:3009897-3011506, chr2:12807024-12809817, chr6:3102201-3103617, chr19:53466821-53467153, chr9:110038258-110039811, chr13:94403032-94404110, chr3:113805901-113842867, and chr19:40715824-40716843.

### Sample

The methods described herein can be applied to samples that contain nucleic acids, preferably a nucleic acid target gene region of interest, from any of a variety of sources, for any of a variety of purposes. Typically the methods used herein are used to determine information regarding a subject, or to determine a relationship between nucleic acid methylation and disease. The samples used in the methods described herein will be selected according to the purpose of the method to be applied. For example, samples can contain nucleic acid from a plurality of different organisms when a phenotype of the organisms is to be correlated with the presence or absence of a methylated nucleic acid molecule or nucleotide locus. In another example, samples can contain nucleic acid from one individual, where the sample is examined to determine the disease state or tendency toward disease of the individual. One skilled in the art can use the methods described herein to determine the desired sample to be examined.

A sample may be from any subject, including for example, animal, plant, bacterium, fungus, virus or parasite. Animal may include for example mammals, birds, reptiles, amphibians or fish. Preferably subject mammals are humans. A sample from a subject can be in any form that provides a desired nucleic acid to be analyzed, including a solid material such as a tissue, cells, a cell pellet, a cell extract, feces, or a biopsy, or a biological fluid such as urine, whole blood, serum, plasma, interstitial fluid, peritoneal fluid, lymph fluids, ascites, sweat, saliva, follicular fluid, breast milk, non-milk breast secretions, cerebral spinal fluid, seminal fluid, lung sputum, amniotic fluid, exudate from a region of infection or inflammation, a mouth wash containing buccal cells, synovial fluid, or any other fluid sample produced by the subject. In addition, the sample can be collected tissues, including bone marrow, epithelium, stomach, prostate, kidney, bladder, breast, colon, lung, pancreas, endometrium, neuron, and muscle. Samples can include tissues, organs, and pathological samples such as a formalin-fixed sample embedded in paraffin.

As one of skill in the art will recognize, some samples may be used directly in the methods provided herein. For example, samples can be examined using the methods described herein without any purification or manipulation steps to increase the purity of desired cells or nucleic acid molecules.

If desired, a sample may be prepared using known techniques, such as that described by Maniatis, et al. (Molecular Cloning: A Laboratory Manual, Cold Spring Harbor, N.Y., pp. 280-281 (1982)). For example, samples examined using the methods described herein can be treated in one or more purification steps in order to increase the purity of the desired cells or nucleic acid in the sample. If desired, solid materials may be mixed with a fluid.

Methods for isolating nucleic acid in a sample from essentially any organism or tissue or organ in the body, as well as from cultured cells are well known. For example, the sample can be treated to homogenize an organ, tissue or cell sample, and the cells may be lysed using known lysis buffers, sonication, electroporation and combinations thereof. Further purification can be performed as needed, as will be appreciated by those skilled in the art. In addition, sample preparation may include a variety of reagents, which can be included in subsequent steps. These include reagents such as salts, buffers, neutral proteins (e.g., albumin), detergents, and such reagents, which can be used to facilitate optimal hybridization or enzymatic reactions, and/or reduce non-specific or background interactions. Also, reagents that otherwise improve the efficiency of the assay, such as, for example, protease inhibitors, nuclease inhibitors and anti-microbial agents, can be used, depending on the sample preparation methods and purity of the nucleic acid target gene molecule.

### Nucleic Acid Target Gene Molecule

The methods provided herein are used to determine methylation states, including whether a nucleic acid target gene molecule contains a methylated or unmethylated nucleotide and determination of methylation ratios (methylated versus unmethylated) for one or more methylation sites or groups of methylation sites. Thus, nucleic acid target gene molecules used in the methods provided herein include any nucleic acid molecule. One or more methods provided herein may be practiced to provide information regarding methylated nucleotides in the nucleic acid target gene molecule.

The methods provided herein permit any nucleic acid-containing sample or specimen, in purified or non-purified form, to be used. Thus, the process may employ for example, DNA or RNA, including messenger RNA, wherein DNA or RNA can be single stranded or double stranded.

The specific nucleic acid sequence to be examined, (i.e., the nucleic acid target gene molecule), may be a fraction of a larger molecule or may be present initially as a discrete molecule, so that the specific nucleic acid target gene molecule constitutes the entire nucleic acid component of a sample, It is not necessary that the nucleic acid target gene molecule to be examined be present initially in a pure form; it may be a minor fraction of a complex mixture, such as contained in whole organism DNA. The nucleic acid target gene molecule for which methylation status is to be determined may be an isolated molecule or part of a mixture of nucleic acid molecules.

The nucleic acid target gene molecule to be analyzed may include one or more protein-encoding regions of genomic DNA or a portion thereof. The nucleic acid target gene molecule can contain one or more gene promoter regions, one or more CpG islands, one or more sequences related to chromatin structure, or other regions of cellular nucleic acid. The nucleic acid target gene molecule can be methylated or unmethylated at individual nucleotides, such as cytosines; at small groups of nucleotides, such as cytosine-rich sequences, or at one or more CpG islands.

The length of the nucleic acid target gene molecule that may be used in the current methods may vary according to the sequence of the nucleic acid target gene molecule, the particular methods used for methylation identification, and the particular methylation state identification desired, but will typically be limited to a length at which fragmentation and detection methods disclosed herein can be used to identify the methylation state of one or more nucleotide loci of the nucleic acid target gene molecule.

In one embodiment, the nucleic acid target gene molecule is of a length in which the methylation state of two or more nucleotide loci can be identified. For example, a nucleic acid target gene molecule may be at least about 20, 25, 30, 35, 40, 50, 60, 70, 80, 90, 100, 120, 140, 160, 180, 200, 225, 250, 275, 300, 350, 400, 450, 500, 550, 600, 700, 800, 900, 1000, 1200, 1400, 1600, 1800, 2000, 2500 or 3000 bases in length. Typically, a nucleic acid target gene molecule will be no longer than about 10,000, 5000, 4000, 3000, 2500, 2000, 1500, 1000, 900, 800, 700, 600, 500, 450, 400, 350, 280, 260, 240, 220, 200, 190, 180, 170, 160, 150, 140, 130, 120, 110 or 100 bases in length.

A nucleic acid target gene molecule examined using the methods disclosed herein may contain one or more methylated nucleotides, but is not required to contain any methylated nucleotides. The methods disclosed herein may be used to identify whether or not a nucleic acid target gene molecule contains methylated or unmethylated nucleotides, to identify the nucleotide locus of a methylated or unmethylated nucleotide in the nucleic acid target gene molecule and to determine the ratio of methylated versus unmethylated nucleotides at one or more methylation sites.

A nucleotide that has been identified as methylated in genomic DNA is cytosine. Methylated cytosines can be present in any of a variety of regions of genomic DNA. The methods provided herein may be used to determine the methylation state of a cytosine in any of a variety of genomic DNA regions. For example, methylcytosine is commonly found in cytosine-guanine dinucleotides termed “CpG” dinucleotides. In one embodiment, the methylation state of a cytosine nucleotide in one or more CpG dinucleotides in the nucleic acid target gene molecule is identified. Such dinucleotides are enriched in some regions of the genome, where these enriched regions are termed CpG islands. CpG islands may be found near promoter regions for some genes, including promoter regions for tumor suppressor genes, oncogenes, developmental regulatory genes, and housekeeping genes. Thus, the methods disclosed herein can be used to identify whether a cytosine in a CpG dinucleotide in a nucleic acid target gene molecule is methylated where the CpG nucleotide is located in a gene promoter region, such as a tumor suppressor gene, oncogene, developmental regulatory gene, or housekeeping gene promoter region. The methods disclosed herein also may be used to identify whether a one or more cytosines in a CpG island in a nucleic acid target gene molecule are methylated.

The methods provided herein may be used to identify the methylation of a plurality of nucleotide loci. Accordingly, methylation of one or more, up to all, nucleotide loci of a large nucleic acid target gene region may be identified using the methods provided herein. For example, the methylation state of a plurality of nucleotide loci, up to all nucleotide loci of an entire CpG island may be identified using the methods provided herein.

Nucleic acid molecules can contain nucleotides with modifications, such as methylation, that do not change the nucleotide sequence of the nucleic acid molecule. Amplification of a nucleic acid molecule containing such a modified nucleotide can result in an amplified product complementary to the unmodified nucleotide, resulting in the amplified product not containing the information regarding the nucleotide modification. For example, the amplified product of a nucleic acid molecule containing a methylated cytosine will result in an amplified product containing either an unmodified guanine (for the complementary strand) or an unmodified cytosine at the location of the methylated cytosine. Reagents are known that can modify the nucleotide sequence of a nucleic acid target gene molecule according to the presence or absence of modifications in one or more nucleotides, where the modification itself does not change the nucleotide sequence. For example, bisulfite may be used in a process to convert unmethylated cytosine into uracil, thus resulting in a modification of the nucleotide sequence of a nucleic acid target gene molecule according to the presence of unmethylated cytosines in the nucleic acid target gene molecule.

In performing the methods disclosed herein, the nucleic acid target gene molecule is treated with a reagent that can modify the nucleic acid target gene molecule as a function of its methylation state. The treated nucleic acid target gene molecule can have a resulting sequence that reflects the methylation state of the untreated nucleic acid target gene molecule. In one embodiment, the reagent can be used to modify an unmethylated selected nucleotide to produce a different nucleotide. For example, the reagent may be used to modify unmethylated cytosine to produce uracil.

### Reagents for Sequence Modification

A method for determining the methylation state of a nucleic acid molecule or nucleotide locus includes contacting a nucleic acid target gene molecule-containing sample with a reagent that can modify the nucleic acid target gene molecule nucleotide sequence as a function of its methylation state. A variety of reagents for modifying the nucleotide sequence of nucleic acid molecules are known in the art and can be used in conjunction with the methods provided herein. For example, a nucleic acid target gene molecule can be contacted with a reagent that modifies unmethylated bases but not methylated bases, such as unmethylated cytosines but not methylated cytosines, in such a manner that the nucleotide sequence of the nucleic acid target gene molecule is modified at the location of an unmethylated base but not at the location of the methylated base, such as at the location of an unmethylated cytosine but not at the location of a methylated cytosine. An exemplary reagent that modifies unmethylated bases but not methylated bases is sodium bisulfite, which modifies unmethylated cytosines but not methylated cytosines.

Methods for modifying a nucleic acid target gene molecule in a manner that reflects the methylation pattern of the nucleic acid target gene molecule are known in the art, as exemplified in U.S. Pat. No. 5,786,146 and U.S. patent publications 20030180779 and 20030082600.

In one embodiment, the reagent can be used to modify unmethylated cytosine to uracil. An exemplary reagent used for modifying unmethylated cytosine to uracil is sodium bisulfite. Sodium bisulfite (NaHSO,) reacts with the 5,6-double bond of cytosine to form a sulfonated cytosine reaction intermediate which is susceptible to deamination, giving rise to a sulfonated uracil. The sulfonate group of the sulfonated uracil can be removed under alkaline conditions, resulting in the formation of uracil. Uracil is recognized as a thymine by DNA polymerase enzymes such as Taq polymerase, and, therefore, upon amplification of the nucleic acid target gene molecule using methods such as PCR, the resultant amplified nucleic acid target gene molecule contains thymine at positions where unmethylated cytosine occurs in the starting template nucleic acid target gene molecule, and the complementary strand contains adenine at positions complementary to positions where unmethylated cytosine occurs in the starting nucleic acid target gene molecule. Further, amplification methods such as PCR can yield an amplified nucleic acid target gene molecule containing cytosine where the starting nucleic acid target gene molecule contains 5-methylcytosine, and the complementary strand maintains guanine at positions complementary to positions where methylated cytosine occurs in the starting nucleic acid target gene molecule. Thus, in amplification methods such as PCR, cytosine in the amplified product can mark the location of 5-methylcytosine, and thymine in the amplified product can mark the location of unmethylated cytosine. Similarly, in the amplified product strands complementary to the treated nucleic acid target gene molecule, guanine can mark the location of 5-methylcytosine and adenine can mark the location of unmethylated cytosine.

Exemplary methods for bisulfite treatment of target DNA can include contacting denatured DNA with a bisulfite solution that also may contain urea and hydroquinone, and incubating the mix for 30 seconds at 95° C. and 15 minutes at 55° C., for 20 cycles. In one alternative method, the bisulfite treatment may be performed in agarose, and precipitation steps may be replaced with dialysis steps (U.S. Pat. No. 6,214,556 and Olek et al., Nucl. Acids Res. 24:5064-66 (1996)). Variations of bisulfite treatment of a nucleic acid target gene molecule are known in the art as exemplified in U.S. Pats. Nos. 5,786,146 and 6,214,556, U.S. patent publication 20030082600, Tost et al., Nucl. Acids Res. 37:e50 (2003), Olek et al., Nucl. Acids Res. 24:5064-66 (1996), and Grunau et al., Nucl. Acids Res. 29:e65 (2001).

In the methods provided herein, a methylation-specific reagent-treated nucleic acid target gene molecule can have a different nucleotide sequence compared to the nucleotide sequence of the nucleic acid target gene molecule prior to treatment. Since the methylation-specific reagent modifies the nucleotide sequence of a nucleic acid target gene molecule as a function of the methylation state of the nucleic acid target gene molecule, the treated nucleic acid target gene molecule will have a nucleotide sequence related to the nucleotide sequence of the untreated nucleic acid target gene molecule, which reflects the methylation state of the untreated nucleic acid target gene molecule.

### Amplification of Treated Nucleic Acid Target Gene Molecule

The methods provided herein also may include a step of amplifying the treated nucleic acid target gene molecule using one or more primers. In one embodiment, at least one primer is a methylation specific primer. In another embodiment, the primer contains one or more nucleotides complementary to the nucleotide treated using the methylation-specific reagent. For example, bisulfite is cytosine specific; when bisulfite is used, a primer used in a method of identifying methylated nucleotides can contain one or more guanine nucleotides. The amplification methods can serve to selectively amplify nucleic acid target gene molecules complementary to the primers while not amplifying one or more other nucleic acid molecules in a nucleic acid sample.

Methylation-specific primers, which are also referred to herein as methylation state specific primers, are designed to distinguish between nucleotide sequences of treated nucleic acid target gene molecules based on the methylation state of one or more nucleotides in the untreated nucleic acid target gene molecule. For example, methylation specific primers may be designed to hybridize to a nucleotide sequence of a reagent-treated nucleic acid target gene molecule arising from a nucleic acid target gene molecule that contained methylated nucleotides in preference to hybridizing to a nucleotide sequence of a reagent-treated nucleic acid target gene molecule arising from a nucleic acid target gene molecule that contained unmethylated nucleotides. Correspondingly, methylation specific primers may be designed to hybridize to a nucleotide sequence of a reagent-treated nucleic acid target gene molecule arising from a nucleic acid target gene molecule that contained unmethylated nucleotides in preference to hybridizing to a nucleotide sequence of a reagent-treated nucleic acid target gene molecule arising from a nucleic acid target gene molecule that contained methylated nucleotides.

The primers used for amplification of the treated nucleic acid target gene molecule in the sample can hybridize to the treated nucleic acid target gene molecule under conditions in which a nucleotide synthesis reaction, such as PCR, can occur. Typically, two or more nucleotide synthesis reaction cycles are performed to produce sufficient quantities of nucleic acid target gene molecule for subsequent steps including fragmentation and detection. In methods of selectively amplifying a nucleic acid target gene molecule using a methylation specific primer, at least one primer used in the amplification method will be methylation specific. Preferably the primers used in the amplification method are not methylation specific.

Primers used in the methods disclosed herein are of sufficient length and appropriate sequence to permit specific primer extension using a nucleic acid target gene molecule template. The primers are typically designed to be complementary to each strand of the nucleic acid target gene molecule to be amplified. The primer can be an oligodeoxyribonucleotide, an oligoribonucleotide, or an oligonucleotide containing both deoxyribonucleotides and ribonucleotides, in some embodiments, a primer can contain one or more nucleotide analogs. The length of primer can vary, depending on any of a variety of factors, including temperature, buffer, desired selectivity and nucleotide composition. The primer can contain at least about 5, 8, 10, 15, 20, 25, 30, 40, 50, 60, 70 or 80 nucleotides, and typically contains no more than about 120, 110, 100, 90, 70, 60, 50, 40, 30, 20 or 10 nucleotides.

The oligonucleotide primers used herein can be prepared using any suitable method, such as conventional phosphotriester and phosphodiester methods or automated embodiments thereof. In one such automated embodiment, diethylphosphoramidites are used as starting materials and can be synthesized as described by Beaucage, et al., Tetrahedron Letters 22:1859-1862 (1981). Methods for synthesizing oligonucleotides on a solid support are known in the art, as exemplified in U.S. Pat. No. 4,458,066.

A primer used in accordance with the disclosed amplification and nucleic acid synthesis methods can specifically hybridize to a nucleic acid target gene molecule.

In methods provided herein, the nucleotide sequence of a nucleic acid target gene molecule can be modified as a function of the methylation state of the nucleic acid target gene molecule. Accordingly, the primer binding region of a methylation-specific reagent-treated nucleic acid target gene molecule that corresponds to a methylation state of a region of an untreated nucleic acid target gene molecule can be a primer binding region whose nucleotide sequence reflects the methylation state of that region in the untreated nucleic acid target gene molecule. For example, a region of an untreated nucleic acid target gene molecule that contains a methylcytosine at the 4th nucleotide and an unmethylated cytosine at the 7th nucleotide can be treated with bisulfite, which will convert the cytosine at the 7^{th} nucleotide to uracil without changing the methylcytosine at the 4^{th} nucleotide; thus, a primer binding region of the treated nucleic acid target gene molecule that corresponds to that region of the untreated nucleic acid target gene molecule will contain a cytosine at the 4th nucleotide and a uracil (or thymine) at the 7th nucleotide, and a primer complementary to such a primer binding region will contain an adenine at the locus complementary to the 4th nucleotide and a guanine at the locus complementary to the 7^{th} nucleotide.

The methylation specific primers may be used in methods to specifically amplify nucleic acid target gene molecules according to the methylation state of the nucleic acid target gene molecule, and to thereby selectively increase the amount of nucleic acid target gene in a sample. Methylation state specific amplification methods include one or more nucleic acid synthesis steps, using one or more methylation specific primers.

In accordance with the methods disclosed herein, a nucleic acid target gene sequence can serve as a template for one or more steps of nucleic acid synthesis. The nucleic acid synthesis step or steps can include primer extension, DNA replication, polymerase chain reaction (PCR), reverse transcription, reverse transcription polymerase chain reaction (RT-PCR), rolling circle amplification, whole genome amplification, strand displacement amplification (SDA), and transcription based reactions.

In one embodiment an amplification step can be performed that can amplify one or more nucleic acids without distinguishing between methylated and unmethylated nucleic acid molecules or loci. Such an amplification step can be performed, for example, when the amount of nucleic acid in a sample is very low and detection of methylated nucleic acid target gene molecules can be improved by a preliminary amplification step that does not distinguish methylated nucleic acid target gene molecules from unmethylated nucleic acid target gene molecules or other nucleic acids in the sample. Typically, such an amplification step is performed subsequent to treating the nucleic acid sample with a reagent that modifies the nucleotide sequence of nucleic acid molecules as a function of the methylation state of the nucleic acid molecules. Although this method does not distinguish according to methylation state, the primers used in such an amplification step nevertheless may be used to increase the amount of nucleic acid molecules of a particular nucleic acid target gene region to be examined relative to the total amount of nucleic acid in a sample. For example, primers can be designed to hybridize to a pre-determined region of a nucleic acid target gene molecule in order to increase the relative amount of that nucleic acid target gene molecule in the sample, but without amplifying the nucleic acid target gene molecule according to the methylation state of the nucleic acid target gene molecule. One skilled in the art may determine the primer used in such a preamplification, or amplification, step according to various known factors and including the desired selectivity of the amplification step and any known nucleotide sequence information.

In the methods of nucleic acid synthesis using a double-stranded nucleic acid molecule, the strands are first separated before any nucleic acid synthetic steps. Following strand separation, one or more primers can be hybridized to one or more treated single-stranded nucleic acid molecules to be amplified, and nucleotide synthesis can be performed to add nucleotides to each primer to form a strand complementary to the strand of the nucleic acid target gene molecule. In one embodiment, nucleic acid synthesis can be performed to selectively amplify one of two strands of a treated nucleic acid target gene molecule. In another embodiment, the step of synthesizing a strand complementary to each strand of a double-stranded treated nucleic acid target gene molecule is performed in the presence of two or more primers, such that at least one primer can hybridize to each strand and prime additional nucleotide synthesis.

In the methods of nucleic acid synthesis using a single-stranded nucleic acid molecule, a primer can be hybridized to the single-stranded nucleic acid molecule to be amplified, and nucleotide synthesis may be performed to add nucleotides to the primer to form a strand complementary to the single-stranded nucleic acid molecule. In one embodiment, the step of synthesizing a strand complementary to a single-stranded nucleic acid molecule is performed in the presence of two or more primers, such that one primer can hybridize to the nucleotide sequence of the strand of the nucleic acid target gene molecule, and one primer can hybridize to the synthesized complementary strand and prime additional nucleotide synthesis. For example, after synthesis of the complementary strand, PCR amplification of the nucleic acid molecule can be immediately performed without further manipulation of the sample.

In another embodiment, the step of synthesizing a strand complementary to a single-stranded nucleic acid molecule is performed separately from additional nucleotide synthetic reactions. For example, the complementary strand can be synthesized to form a double-stranded nucleic acid molecule, and the sample may be subjected to one or more intermediate steps prior to amplifying the double-stranded nucleic acid molecule. Intermediate steps may include any of a variety of methods of manipulating a nucleic acid sample, including increasing the purity of the nucleic acid molecule, removing excess primers, changing the reaction conditions (e.g., the buffer conditions, enzyme or reactants present in the sample), and other parameters. In one example, the sample may be subjected to one or more purification steps of the nucleic acid molecule. For example, the primer used to create the strand complementary to the nucleic acid molecule can contain a moiety at its 5′ end that permits identification or isolation of the primer or of a nucleic acid into which the primer is incorporated. Such a moiety may be, for example, a bindable moiety such as biotin, polyhistidine, magnetic bead, or other suitable substrate, whereby contacting the sample with the binding partner of the bindable moiety may result in selective binding of nucleic acid molecule into which the primer has been incorporated. Such selective binding may be used to separate the nucleic acid molecule from sample impurities, thereby increasing the purity of the nucleic acid molecule. After performing one or more intermediate steps, such as purity enhancing steps, the nucleic acid molecule may be amplified according to the methods provided herein and as known in the art.

After formation of the strand complementary to the single-stranded nucleic acid target gene molecules, subsequent nucleic acid target gene molecule amplification steps may be performed in which the complementary strands are separated, primers are hybridized to the strands, and the primers have added thereto nucleotides to form a new complementary strand. Strand separation may be effected either as a separate step or simultaneously with the synthesis of the primer extension products. This strand separation may be accomplished using various suitable denaturing conditions, including physical, chemical, or enzymatic means, the word “denaturing” includes all such means. One physical method of separating nucleic acid strands involves heating the nucleic acid target gene molecule until it is denatured. Typical heat denaturation may involve temperatures ranging from about 80° C. to 105° C., for times ranging from about 1 to 10 minutes. Strand separation also may be accomplished by chemical means, including high salt conditions or strongly basic conditions. Strand separation also may be induced by an enzyme from the class of enzymes known as helicases or by the enzyme RecA, which has helicase activity, and in the presence of riboATP, is known to denature DNA. The reaction conditions suitable for strand separation of nucleic acids with helicases are described by Kuhn Hoffmann-Berling, CSH-Quan tita rive Biology, 43:63 (1978) and techniques for using RecA are reviewed in C. Radding, Ann. Rev. Genetics 16:405-437 (1982).

After each amplification step, the amplified product will be double stranded, with each strand complementary to the other. The complementary strands of may be separated, and both separated strands may be used as a template for the synthesis of additional nucleic acid strands. This synthesis may be performed under conditions allowing hybridization of primers to templates to occur. Generally synthesis occurs in a buffered aqueous solution, typically at about a pH of 7-9, such as about pH 8. Typically, a molar excess of two oligonucleotide primers can be added to the buffer containing the separated template strands. In some embodiments, the amount of target nucleic acid is not known (for example, when the methods disclosed herein are used for diagnostic applications), so that the amount of primer relative to the amount of complementary strand cannot be determined with certainty.

In an exemplary method, deoxyribonucleoside triphosphates DATP, dCTP, dGTP, and dTTP can be added to the synthesis mixture, either separately or together with the primers, and the resulting solution can be heated to about 90° C.-100° C. from about 1 to 10 minutes, typically from 1 to 4 minutes. After this heating period, the solution can be allowed to cool to about room temperature. To the cooled mixture can be added an appropriate enzyme for effecting the primer extension reaction (called herein “enzyme for polymerization”), and the reaction can be allowed to occur under conditions known in the art. This synthesis (or amplification) reaction can occur at room temperature up to a temperature above which the enzyme for polymerization no longer functions. For example, the enzyme for polymerization also may be used at temperatures greater than room temperature if the enzyme is heat stable. In one embodiment, the method of amplifying is by PCR, as described herein and as is commonly used by those of skill in the art. Alternative methods of amplification have been described and also may be employed. A variety of suitable enzymes for this purpose are known in the art and include, for example, E. coli DNA polymerase I, Klenow fragment of E. coli DNA polymerase I, T4 DNA polymerase, other available DNA polymerases, polymerase muteins, reverse transcriptase, and other enzymes, including thermostable enzymes (i.e., those enzymes which perform primer extension at elevated temperatures, typically temperatures that cause denaturation of the nucleic acid to be amplified).

### Manipulation of Both Strands of a Nucleic Acid Target Gene Molecule

Methods of manipulating a nucleic acid target gene molecule subsequent to methylation-based sequence modification treatment, such as amplification and fragmentation, may be performed using only one strand of the treated nucleic acid target gene molecule, or using both strands of the treated nucleic acid target gene molecule. For example, primers used for amplification steps may be complementary to only one strand of the treated nucleic acid target gene molecule, or may be complementary to both strands of the treated nucleic acid. Accordingly, amplification steps may be performed to create at least two different amplified double-stranded products, where both strands of the treated nucleic acid target gene molecule is amplified into separate double-stranded products.

Alternatively, amplification may be performed such that only one of the two strands of the treated nucleic acid target gene molecule is amplified. For example, when amplification is performed using at least one primer that is selective for the sequence of one of the two strands, the strand hybridized to the primer may be selectively amplified.

After one or more steps of amplification, the amplified products may be subjected to one or more manipulation steps prior to additional amplification steps or prior to cleavage steps. For example, amplified products can be subjected to one or more purification steps prior to additional amplification or prior to cleavage.

Methods for purifying nucleic acid molecules are known in the art and include precipitation, dialysis or other solvent exchange, gel electrophoresis, enzymatic degradation of impurities (e.g., protease treatment, or RNase treatment for a DNA nucleic acid target gene molecule sample), liquid chromatography including ion exchange chromatography and affinity chromatography, and other methods of specifically binding nucleic acid target gene molecules to separate them from impurities (e.g., hybridization, biotin binding). Purification steps also may include separating complementary strands of amplification products. One skilled in the art will know to select which, if any, purification steps to use according to desired level of purity and/or desired sample composition for subsequent amplification, modification or cleavage steps.

Methods for determining methylation in a nucleic acid target gene may include methods in which a single sample is treated in one or more steps, and then the single sample may be divided into two or more aliquots for parallel treatment in subsequent steps.

Amplified products may be split into two or more aliquots after amplification. For example, amplified products may be split into two or more aliquots after amplification but prior to cleaving the amplified products, amplified products may split into two or more aliquots after amplification and subjected to further steps such as one or more amplified product purification steps.

When amplified products are split into two or more aliquots prior to cleavage, different cleavage methods may be applied to each of the two or more aliquots. For example, a first nucleic acid target gene molecule aliquot may be base specifically fragmented with RNase A, while a second nucleic acid target gene molecule aliquot may be base specifically fragmented with Rnase T1. In another example, amplified nucleic acid target gene molecule may be split into four aliquots and each aliquot may be treated with a different base-specific reagent to produce four different sets of base specifically cleaved nucleic acid target gene molecule fragments. Separation into two or more aliquots permits different cleavage reactions to be performed on the same amplification product. Use of different cleavage reactions on the same amplification product is further described in the cleavage methods provided herein.

A sample may be divided into two or more aliquots in specifically amplifying different strands of a nucleic acid target gene molecule in different aliquots. For example, a treated nucleic acid target gene molecule can have non-complementary strands that can be separately treated with different primers such as different methylation state specific primers in separately amplifying the different strands in different aliquots. In another embodiment, complementary strands of an amplified nucleic acid target gene molecule can be separately amplified in different aliquots, according to the primers used in each aliquot. For example, a sample of amplified nucleic acid target gene molecules can be separated into two or more aliquots, where the forward strand is transcribed in a first set of aliquots and the reverse strand is transcribed in a second set of aliquots. As will be appreciated by one skilled in the art, a sample can be divided into any of a plurality of aliquots in which any combination of the parallel reactions described herein may be performed.

Fragmentation in Conjunction with Nucleotide Synthesis

Selective nucleotide synthesis also may be performed in conjunction with fragmentation. A nucleic acid target gene amplified through a plurality of nucleic acid synthesis cycles will utilize primers hybridizing to two separate regions of the nucleic acid target gene molecule. Fragmentation of a nucleic acid target gene molecule in the center region in between the two primer hybridization sites will prevent amplification of the nucleic acid target gene molecule. Hence selective fragmentation of the center region of nucleic acid molecules may result in selective amplification of a nucleic acid target gene molecule even if the primers used in the nucleic acid synthesis reactions are not selective.

In one example, the sample may be treated with fragmentation conditions prior to being treated with nucleic acid synthesis conditions, and prior to being treated with a reagent that modifies the nucleic acid target gene molecule sequence as a function of the methylation state of the nucleic acid target gene. In such an example, the fragmentation conditions may be selective for methylated or unmethylated nucleotides. For example, a sample can have added thereto a methylation sensitive endonuclease, such as HPAII, which cleaves at an unmethylated recognition site but not at a methylated recognition site. This results in a sample containing intact nucleic acid target gene molecules that are methylated at the recognition site and cleaved nucleic acid target gene molecules that are unmethylated at the recognition site. The sample then may be treated with nucleic acid synthesis conditions using primers designed so that only uncleaved nucleic acid target gene molecules are amplified. As a result of the cleavage, amplification will be selective for nucleic acid target gene molecules that are methylated at the recognition site.

In another example, the sample may be treated with fragmentation conditions prior to treatment with nucleic acid synthesis conditions, but subsequent to treatment with a reagent that modifies the nucleic acid target gene molecule sequence as a function of the methylation state of the nucleic acid target gene. For example, a sample can have added thereto an endonuclease that cleaves at a recognition site that includes a C nucleotide at a particular locus, but not a recognition site that contains a T or U nucleotide at that particular locus. Or vice versa, a sample can have added thereto an endonuclease that cleaves at a recognition site that includes a T or U nucleotide at a particular locus, but not a recognition site that contains a C nucleotide at that particular locus. The sample can first be treated with a reagent that modifies the nucleic acid target gene molecule sequence as a function of the methylation state of the nucleic acid target gene molecule, and then treated with such an endonuclease. The resulting sample will contain intact nucleic acid target gene molecules that have the desired methylation state at the recognition site and cleaved nucleic acid target gene molecules that have the undesired methylation state at the recognition site. The sample then can be treated with nucleic acid synthesis conditions using primers designed so that only uncleaved nucleic acid target gene molecules are amplified. As a result of the cleavage, amplification will be selective for nucleic acid target gene molecules that are methylated at the recognition site.

### Transcription

Transcription of template DNA such as a nucleic acid target gene molecule, or an amplified product thereof, may be performed for one strand of the template DNA or for both strands of the template DNA. In one embodiment, the nucleic acid molecule to be transcribed contains a moiety to which an enzyme capable of performing transcription can bind; such a moiety may be, for example, a transcriptional promoter sequence.

Transcription reactions may be performed using any of a variety of methods known in the art, using any of a variety of enzymes known in the art. For example, mutant T7 RNA polymerase (T7 R&DNA polymerase; Epicentre, Madison, Wis.) with the ability to incorporate both dNTPs and rNTPs may be used in the transcription reactions. The transcription reactions may be run under standard reaction conditions known in the art, for example, 40 mM Tris-Ac (pH 7.51, 10 mM NaCl, 6 mM MgCl, 2 mM spermidine, 10 mM dithiothreitol, 1 mM of each rNTP, 5 mM of dNTP (when used), 40 nM DNA template, and 5 U/uL T7 R&DNA polymerase, incubating at 37° C. for 2 hours. After transcription, shrimp alkaline phosphatase (SAP) may be added to the cleavage reaction to reduce the quantity of cyclic monophosphate side products. Use of T7 R&DNA polymerase is known in the art, as exemplified by U.S. Pat. Nos. 5,849,546 and 6,107,037, and Sousa et al., EMBO J. 14:4609-4621 (1995), Padilla et al., Nucl. Acid Res. 27:1561-1563 (1999), Huang et al., Biochemistry 36:8231-8242 (1997), and Stanssens et al., Genome Res., 14:126-133 (2004).

In addition to transcription with the four regular ribonucleotide substrates (rCTP, rATP, rGTP and rUTP), reactions may be performed replacing one or more ribonucleoside triphosphates with nucleoside analogs, such as those provided herein and known in the art, or with corresponding deoxyribonucleoside triphosphates (e.g., replacing rCTP with dCTP, or replacing rUTP with either dUTP or dTTP). In one embodiment, one or more rNTPs are replaced with a nucleoside or nucleoside analog that, upon incorporation into the transcribed nucleic acid, is not cleavable under the fragmentation conditions applied to the transcribed nucleic acid.

In one embodiment, transcription is performed subsequent to one or more nucleic acid synthesis reactions, including one or more nucleic acid synthesis reactions using methylation specific primers. For example, transcription of an amplified product can be performed subsequent to amplification of a nucleic acid target gene molecule, including methylation specific amplification of the nucleic acid target gene molecule. In another embodiment, the treated nucleic acid target gene molecule is transcribed without any preceding nucleic acid synthesis steps.

### Fragmentation of Nucleic Acid Molecules

The methods provided herein also include steps of fragmentation and/or cleavage of nucleic acid target gene molecules or amplified products. Any method for cleaving a nucleic acid molecule into fragments with a suitable fragment size distribution may be used to generate the nucleic acid fragments. Fragmentation of nucleic acid molecules is known in the art and may be achieved in many ways. For example, nucleic acid molecules composed of DNA, RNA, analogs of DNA and RNA or combinations thereof, can be fragmented physically, chemically, or enzymatically. In one embodiment, enzymatic cleavage at one or more specific cleavage sites can be used to produce the nucleic acid molecule fragments utilized herein. Typically, cleavage is effected after amplification such that once a sufficient quantity of amplified products is generated using the methods provided herein, the amplified products can be cleaved into two or more fragments.

In embodiments where restriction enzymes are used, depending on the number and type of restriction enzymes used and the particular reaction conditions selected, the average length of fragments generated may be controlled within a specified range. In particular embodiments, fragments of nucleic acid molecules prepared for use herein may range in size from the group of ranges including about 1-50 bases, about 2-40 bases, about 3-35 bases, and about 5-30 bases. Yet other size ranges contemplated for use herein include between about 50 to about 150 bases, from about 25 to about 75 bases, or from about 12-30 bases. In one particular embodiment, fragments of about 3 to about 35 bases are used. Generally, fragment size range will be selected so that the mass of the fragments can be accurately determined using the mass measurement methods described herein and known in the art; also in some embodiments, size range is selected in order to facilitate the desired desorption efficiencies in MALDI-TOF MS.

Base-specific fragmentation using nucleases is a preferred fragmentation method. Nucleic acid target gene molecules may be fragmented using nucleases that selectively cleave at a particular base (e.g., A, C, T or G for DNA and A, C, U or G for RNA) or base type (i.e., pyrimidine or purine). In one embodiment, RNases that specifically cleave 3 RNA nucleotides (e.g., U, G and A), 2 RNA nucleotides (e.g., C and U) or 1 RNA nucleotide (e.g., A), may be used to base specifically cleave transcripts of a nucleic acid target gene molecule. For example, RNase T1 cleaves ssRNA (single-stranded RNA) at G ribonucleotides, RNase U2 digests ssRNA at A ribonucleotides, RNase CL3 and cusativin cleave ssRNA at C ribonucleotides, PhyM cleaves ssRNA at U and A ribonucleotides, and RNAse A cleaves ssRNA at pyrimidine ribonucleotides (C and U). The use of mono-specific Rnases such as RNase T, (G specific) and RNase U, (A specific) is known in the art (Donis-Keller et al., Nucl. Acids Res. 4:2527-2537 (1977); Gupta and Randerath, Nucl. Acids Res. 4:1957-1978 (1977); Kuchino and Nishimura, Methods Enzymol. 180:154-163 (1989); and Hahner et al., Nucl. Acids Res. 25(10): 1957-1964 (1997)). Another enzyme, chicken liver ribonuclease (RNase CL3) has been reported to cleave preferentially at cytidine, but the enzyme's proclivity for this base has been reported to be affected by the reaction conditions (Boguski et al., J. Biol. Chem. 255:2160-2163 (1980)). Reports also claim cytidine specificity for another ribonuclease, cusativin, isolated from dry seeds of Cucumis sativus L (Rojo et al., Planta 194:328-338 (1994)). Alternatively, the identification of pyrimidine residues by use of RNase PhyM (A and U specific) (Donis-Keller, H. Nucleic Acids Res. 8:3133-3142 (1980)) and RNase A (C and U specific) (Simoncsits et al., Nature 269:833-836 (1977); Gupta and Randerath, Nucl. Acids Res. 4:1957-1978 (1977)) has been demonstrated. Examples of such cleavage patterns are given in Stanssens et al., WO 00/66771.

Base specific cleavage reaction conditions using an RNase are known in the art, and can include, for example 4 mM Tris-Ac (pH 8.01, 4 mM KAc, 1 mM spermidine, 0.5 mM dithiothreitol and 1.5 mM MgCl.

In one embodiment, amplified product can be transcribed into a single stranded RNA molecule and then cleaved base specifically by an endoribonuclease. Treatment of the target nucleic acid, for example using bisulfite which converts unmethylated cytosine to uracil without modifying methylated cytosine, can be used to generate differences in base specific cleavage patterns that can be analyzed by mass analysis methods, such as mass spectrometry, and can be used for identification of methylated sites. In one embodiment, transcription of a nucleic acid target gene molecule can yield an RNA molecule that can be cleaved using specific RNA endonucleases. For example, base specific cleavage of the RNA molecule can be performed using two different endoribonucleases, such as RNAse T1 and RNAse A. RNAse T1 specifically cleaves G nucleotides, and RNAse A specifically cleaves pyrimidine ribonucleotides (i.e., cytosine and uracil residues). In one embodiment, when an enzyme that cleaves more than one nucleotide, such as RNAse A, is used for cleavage, non-cleavable nucleosides, such as dNTP's may be incorporated during transcription of the nucleic acid target gene molecule or amplified product. For example, dCTPs may be incorporated during transcription of the amplified product, and the resultant transcribed nucleic acid can be subject to cleavage by RNAse A at U ribonucleotides, but resistant to cleavage by RNAse A at C deoxyribonucleotides. In another example, dTTPs can be incorporated during transcription of the nucleic acid target gene molecule, and the resultant transcribed nucleic acid can be subject to cleavage by RNAse A at C ribonucleotides, but resistant to cleavage by RNAse A at T deoxyribonucleotides. By selective use of non-cleavable nucleosides such as dNTPs, and by performing base specific cleavage using RNases such as RNAse A and RNAse T1, base cleavage specific to three different nucleotide bases can be performed on the different transcripts of the same target nucleic acid sequence. For example, the transcript of a particular nucleic acid target gene molecule can be subjected to G-specific cleavage using RNAse T1; the transcript can be subjected to C-specific cleavage using dTTP in the transcription reaction, followed by digestion with RNAse A; and the transcript can be subjected to T-specific cleavage using dCTP in the transcription reaction, followed by digestion with RNAse A. These types of base specific cleavage patterns are exemplified below showing the theoretical cleavage products of a given nucleotide sequence TAACGCAT converted through bisulfite treatment to the sequence TAAACGTAT if methylated at the cytosine and to TAAATGTAT if not methylated.

| | Non-methylated | Type of | Methylated |
|---|---|---|---|
| | TAAATGTAT | change | TAAACGTAT |
| RNAse A | TAAATGTAT | Introduction | TAAAC GTAT |
| C specific | | of cleavage | |
| cleavage | | nucleotide | |
| RNAse A | T AAAT GT AT | Removal | T AAACGT AT |
| T specific | | of cleavage | |
| cleavage | | nucleotide | |
| RNAse T1 | TAAATG TAT | Mass Shift | TAAACG TAT |
| G specific | | | |
| cleavage | | | |

In another embodiment, the use of dNTPs, different RNAses, and both orientations of the nucleic acid target gene molecule can allow for six different cleavage schemes. For example, a double stranded nucleic acid target gene molecule can yield two different single stranded transcription products, which can be referred to as a transcript product of the forward strand of the nucleic acid target gene molecule and a transcript product of the reverse strand of the nucleic acid target gene molecule. Each of the two different transcription products can be subjected to three separate base specific cleavage reactions, such as G-specific cleavage, C-specific cleavage and T-specific cleavage, as described herein, to result in six different base specific cleavage reactions. The six possible cleavage schemes are listed below.

| | Forward Primer | Reverse primer |
|---|---|---|
| RNAse T1 | G specific cleavage | G specific cleavage |
| RNAse A: dCTP | T specific cleavage | T specific cleavage |
| RNAse A: dCTP | C specific cleavage | C specific cleavage |

Use of four different base specific cleavage reactions can yield information on all four nucleotide bases of one strand of the nucleic acid target gene molecule. That is, by taking into account that cleavage of the forward strand can be mimicked by cleaving the complementary base on the reverse strand, base specific cleavage can be achieved for each of the four nucleotides of the forward strand by reference to cleavage of the reverse strand. For example, the three base-specific cleavage reactions can be performed on the transcript of the nucleic acid target gene molecule forward strand, to yield G-, C- and T-specific cleavage of the nucleic acid target gene molecule forward strand; and a fourth base specific cleavage reaction can be a T-specific cleavage reaction of the transcript of the nucleic acid target gene molecule reverse strand, the results of which will be equivalent to A-specific cleavage of the transcript of the nucleic acid target gene molecule forward strand. One skilled in the art will appreciate that base specific cleavage to yield information on all four nucleotide bases of one nucleic acid target gene molecule strand can be accomplished using a variety of different combinations of possible base specific cleavage reactions, including cleavage reactions listed above for RNases T1 and A, and additional cleavage reactions for forward or reverse strands and/or using non-hydrolyzable nucleotides can be performed with other base specific RNases known in the art or disclosed herein.

In one example, RNAse U2 can be used to base specifically cleave nucleic acid target gene molecule transcripts. RNAse U2 can base specifically cleave RNA at A nucleotides. Thus, by use of RNAses T1, U2 and A, and by use of the appropriate dNTPs (in conjunction with use of RNase A), all four base positions of a nucleic acid target gene molecule can be examined by base specifically cleaving transcript of only one strand of the nucleic acid target gene molecule. In some embodiments, non-cleavable nucleoside triphosphates are not required when base specific cleavage is performed using RNAses that base specifically cleave only one of the four ribonucleotides. For example, use of RNAse T1, RNase CL3, cusativin, or RNAse U2 for base specific cleavage does not require the presence of non-cleavable nucleotides in the nucleic acid target gene molecule transcript. Use of RNAses such as RNAse T1 and RNAse U2 can yield information on all four nucleotide bases of a nucleic acid target gene molecule. For example, transcripts of both the forward and reverse strands of a nucleic acid target gene molecule or amplified product can be synthesized, and each transcript can be subjected to base specific cleavage using RNAse T1 and RNAse U2. The resulting cleavage pattern of the four cleavage reactions will yield information on all four nucleotide bases of one strand of the nucleic acid target gene molecule. In such an embodiment, two transcription reactions can be performed: a first transcription of the forward nucleic acid target gene molecule strand and a second of the reverse nucleic acid target gene molecule strand.

Also contemplated for use in the methods are a variety of different base specific cleavage methods. A variety of different base specific cleavage methods are known in the art and are described herein, including enzymatic base specific cleavage of RNA, enzymatic base specific cleavage of modified DNA, and chemical base specific cleavage of DNA. For example enzymatic base specific cleavage, such as cleavage using uracil-deglycosylase (UDG) or methylcytosine deglycosylase (MCDG), are known in the art and described herein, and can be performed in conjunction with the enzymatic RNAse-mediated base specific cleavage reactions described herein.

Methods for using restriction endonucleases to fragment nucleic acid molecules are widely known in the art. In one exemplary protocol a reaction mixture of 20-50 ul is prepared containing; DNA 1-3 ug; restriction enzyme buffer 1×; and a restriction endonuclease 2 units for 1 ug of DNA. Suitable buffers also are known in the art and include suitable ionic strength, cofactors, and optionally, pH buffers to provide optimal conditions for enzymatic activity. Specific enzymes may require specific buffers that are generally available from commercial suppliers of the enzyme. An exemplary buffer is potassium glutamate buffer (KGB). Hannish, J. and M. McClelland, “Activity of DNA modification and restriction enzymes in KGB, a potassium glutamate buffer,” Gene Anal. Tech 5:105 (1988); McClelland, M. et al., “A single buffer for all restriction endonucleases,” Nucl. Acids Res. 16:364 (1988). The reaction mixture is incubated at 37° C. for 1 hour or for any time period needed to produce fragments of a desired size or range of sizes. The reaction may be stopped by heating the mixture at 65° C. or 80° C. as needed. Alternatively, the reaction may be stopped by chelating divalent cations such as Mg²⁺ with for example, EDTA.

DNAses also may be used to generate nucleic acid molecule fragments. Anderson, S., “Shotgun DNA sequencing using cloned Dnase I-generated fragments,” Nucl. Acids Res. 9:3015-3027 (1981). DNase I (Deoxyribonuclease I) is an endonuclease that non-specifically digests double- and single-stranded DNA into poly- and mono-nucleotides.

Catalytic DNA and RNA are known in the art and can be used to cleave nucleic acid molecules to produce nucleic acid molecule fragments. Santoro, S. W. and Joyce, G. F. “A general purpose RNA-cleaving DNA enzyme,” Proc. Natl. Acad. Sci. USA 94:4262-4266 (1997). DNA as a single-stranded molecule can fold into three-dimensional structures similar to RNA, and the 2′-hydroxy group is dispensable for catalytic action. As ribozymes, DNAzymes also can be made, by selection, to depend on a cofactor. This has been demonstrated for a histidine-dependent DNAzyme for RNA hydrolysis. U.S. Pat. Nos. 6,326,174 and 6,194,180 disclose deoxyribonucleic acid enzymes, catalytic and enzymatic DNA molecules, capable of cleaving nucleic acid sequences or molecules, particularly RNA.

Fragmentation of nucleic acid molecules may be achieved using physical or mechanical forces including mechanical shear forces and sonication. Physical fragmentation of nucleic acid molecules may be accomplished, for example, using hydrodynamic forces. Typically nucleic acid molecules in solution are sheared by repeatedly drawing the solution containing the nucleic acid molecules into and out of a syringe equipped with a needle. Thorstenson, Y. R. et al., “An Automated Hydrodynamic Process for Controlled, Unbiased DNA Shearing,” Genome Research 8:848-855 (1998); Davison, P. F. Proc. Natl. Acad. Sci. USA 45:1560-1568 (1959); Davison, P. F. Nature 185:918-920 (1960); Schriefer, L. A. et al., “Low pressure DNA shearing: a method for random DNA sequence analysis,” Nucl. Acids Res. 18:7455-7456 (1990). Shearing of DNA, for example with a hypodermic needle, typically generates a majority of fragments ranging from 1-2 kb, although a minority of fragments can be as small as 300 bp.

The hydrodynamic point-sink shearing method developed by Oefner et al., is one method of shearing nucleic acid molecules that utilizes hydrodynamic forces. Oefner, P. J. et al., “Efficient random subcloning of DNA sheared in a recirculating point-sink flow system,” Nucl. Acids Res. 24(20):3879-3886 (1996).

Nucleic acid molecule fragments also may be obtained by agitating large nucleic acid molecules in solution, for example by mixing, blending, stirring, or vortexing the solution. Hershey, A. D. and Burgi, E. J. Mol. Biol, 2:143-152 (1960); Rosenberg, H. S, and Bendich, A. J. Am. Chem. Soc. 82:3198-3201 (1960).

One suitable method of physically fragmenting nucleic acid molecules is based on sonicating the nucleic acid molecule. Deininger, P. L. “Approaches to rapid DNA sequence analysis,” Anal. Biochem. 129:216-223 (1983).

Fragmentation of nucleic acid molecules also may be achieved using a nebulizer. Bodenteich, A., Chissoe, S., Wang, Y.-F. and Roe, B. A. (1994) In Adams, M. D., Fields, C. and Venter, J. C. (eds.) Automated DNA Sequencing and Analysis. Academic Press, San Diego, Calif. Nebulizers are known in the art and commercially available.

Another method for fragmenting nucleic acid molecule employs repeatedly freezing and thawing a buffered solution of nucleic acid molecules. The sample of nucleic acid molecules may be frozen and thawed as necessary to produce fragments of a desired size or range of sizes.

Nucleic acid molecule fragmentation also may be achieved by irradiating the nucleic acid molecules. Typically, radiation such as gamma or x-ray radiation will be sufficient to fragment the nucleic acid molecules.

Chemical fragmentation may be used to fragment nucleic acid molecules either with base specificity or without base specificity. Nucleic acid molecules may be fragmented by chemical reactions including for example, hydrolysis reactions including base and acid hydrolysis. An exemplary acid/base hydrolysis protocol for producing nucleic acid molecule fragments are known (see, e.g., Sargent et al., Meth. Enz. 152:432 (1988)).

### Mass Spectrometry

When analyses are performed using mass spectrometry, such as MALDI, nanoliter volumes of sample can be loaded on chips. Use of such volumes can permit quantitative or semi-quantitative mass spectrometric results. For example, the area under the peaks in the resulting mass spectra are proportional to the relative concentrations of the components of the sample. Methods for preparing and using such chips are known in the art, as exemplified in U.S. Pat. No. 6,024,925, U.S. Publication 20010008615, and PCT Application No. PCT/US97/20195 (WO 98/20020); methods for preparing and using such chips also are provided in co-pending U.S. application Ser. Nos. 08/786,988, 09/364,774, and 09/297,575. Chips and kits for performing these analyses are commercially available from SEQUENOM under the trademark MassARRAY′″. MassARRAY′″ systems contain a miniaturized array such as a SpectroCHIP@ useful for MALDI-TOF (Matrix-Assisted Laser Desorption Ionization-Time of Flight) mass spectrometry to deliver results rapidly. It accurately distinguishes single base changes in the size of DNA fragments relating to genetic variants without tags.

In one embodiment, the mass of all nucleic acid molecule fragments formed in the step of fragmentation is measured. The measured mass of a nucleic acid target gene molecule fragment or fragment of an amplification product also can be referred to as a “sample” measured mass, in contrast to a “reference” mass which arises from a reference nucleic acid fragment.

In another embodiment, the length of nucleic acid molecule fragments whose mass is measured using mass spectroscopy is no more than 75 nucleotides in length, no more than 60 nucleotides in length, no more than 50 nucleotides in length, no more than 40 nucleotides in length, no more than 35 nucleotides in length, no more than 30 nucleotides in length, no more than 27 nucleotides in length, no more than 25 nucleotides in length, no more than 23 nucleotides in length, no more than 22 nucleotides in length, no more than 21 nucleotides in length, no more than 20 nucleotides in length, no more than 19 nucleotides in length, or no more than 18 nucleotides in length. In another embodiment, the length of the nucleic acid molecule fragments whose mass is measured using mass spectroscopy is no less than 3 nucleotides in length, no less than 4 nucleotides in length, no less than 5 nucleotides in length, no less than 6 nucleotides in length, no less than 7 nucleotides in length, no less than 8 nucleotides in length, no less than 9 nucleotides in length, no less than 10 nucleotides in length, no less than 12 nucleotides in length, no less than 15 nucleotides in length, no less than 18 nucleotides in length, no less than 20 nucleotides in length, no less than 25 nucleotides in length, no less than 30 nucleotides in length, or no less than 35 nucleotides in length.

In one embodiment, the nucleic acid molecule fragment whose mass is measured is RNA. In another embodiment the nucleic acid target gene molecule fragment who's mass is measured is DNA. In yet another embodiment, the nucleic acid target gene molecule fragment whose mass is measured contains one modified or atypical nucleotide (i.e., a nucleotide other than deoxy-C, T, G or A in DNA, or other than C, U, G or A in RNA). For example, a nucleic acid molecule product of a transcription reaction may contain a combination of ribonucleotides and deoxyribonucleotides. In another example, a nucleic acid molecule can contain typically occurring nucleotides and mass modified nucleotides, or can contain typically occurring nucleotides and non-naturally occurring nucleotides.

Prior to mass spectrometric analysis, nucleic acid molecules can be treated to improve resolution. Such processes are referred to as conditioning of the molecules. Molecules can be “conditioned,” for example to decrease the laser energy required for volatilization and/or to minimize fragmentation. A variety of methods for nucleic acid molecule conditioning are known in the art. An example of conditioning is modification of the phosphodiester backbone of the nucleic acid molecule (e.g., by cation exchange), which can be useful for eliminating peak broadening due to a heterogeneity in the cations bound per nucleotide unit. In another example, contacting a nucleic acid molecule with an alkylating agent such as alkyloidide, iodoacetamide, P-iodoethanol, or 2,3-epoxy-1-propanol, can transform a monothio phosphodiester bonds of a nucleic acid molecule into a phosphotriester bond. Likewise, phosphodiester bonds can be transformed to uncharged derivatives employing, for example, trialkylsilyl chlorides. Further conditioning can include incorporating nucleotides that reduce sensitivity for depurination (fragmentation during MS) e.g., a purine analog such as N7- or N9-deazapurine nucleotides, or RNA building blocks or using oligonucleotide triesters or incorporating phosphorothioate functions which are alkylated, or employing oligonucleotide mimetics such as PNA.

For some applications, simultaneous detection of more than one nucleic acid molecule fragment may be performed. In other applications, parallel processing can be performed using, for example, oligonucleotide or oligonucleotide mimetic arrays on various solid supports. “Multiplexing” can be achieved by several different methodologies. For example, fragments from several different nucleic acid molecules can be simultaneously subjected to mass measurement methods. Typically, in multiplexing mass measurements, the nucleic acid molecule fragments should be distinguishable enough so that simultaneous detection of the multiplexed nucleic acid molecule fragments is possible. Nucleic acid molecule fragments may be made distinguishable by ensuring that the masses of the fragments are distinguishable by the mass measurement method to be used. This may be achieved either by the sequence itself (composition or length) or by the introduction of mass-modifying functionalities into one or more nucleic acid molecules.

In one embodiment, the nucleic acid molecule to be mass-measured contains attached thereto one or more mass-modifying moieties. Mass-modifying moieties are known in the art and may be attached to the 3′ end or 5′ end of a nucleic acid molecule fragment, may be attached to a nucleobase or to a sugar moiety of a nucleotide, or may be attached to or substitute for the phosphodiester linkage between nucleotides. A simple mass-modification may be achieved by substituting H for halogens like F, Cl, Br and/or I, or pseudohalogens such as SCN, NCS, or by using different alkyl, aryl or aralkyl moieties such as methyl, ethyl, propyl, isopropyl, t-butyl, hexyl, phenyl, substituted phenyl, benzyl, or functional groups such as N₃, CH₂F, CHF₂, CF₃, Si(CH₃)₃, Si(CH₃)₂, (C₂H₅), Si(CH₃)(C₂H₅)₂, Si(C₂H₅)₃. Yet another mass-modification can be obtained by attaching homo- or heteropeptides through the nucleic acid molecule (e.g., detector (D)) or nucleoside triphosphates. One example useful in generating mass-modified species with a mass increment of 57 is the attachment of oligoglycines, e.g., mass-modifications of 74, 131, 188, 245 are achieved. Simple oligoamides also can be used, e.g., mass-modifications of 74, 88, 102, 116 . . . , are obtainable.

Mass-modifications also may include oligo/polyethylene glycol derivatives. The oligo/polyethylene glycols also can be monoalkylated by a lower alkyl such as methyl, ethyl, propyl, isopropyl, t-butyl and other suitable substituents. Other chemistries also can be used in the mass-modified compounds (see, e.g., those described in Oligonucleotides and Analogues, A Practical Approach, F. Eckstein, editor, IRL Press, Oxford, 1991).

Mass modifying moieties can be attached, for instance, to either the 5′-end of the oligonucleotide, to the nucleobase (or bases), to the phosphate backbone, to the 2′-position of the nucleoside (nucleosides), and/or to the terminal 3′-position. Examples of mass modifying moieties include, for example, a halogen, an azido, or of the type, XR, wherein X is a linking group and R is a mass-modifying functionality. A mass-modifying functionality can, for example, be used to introduce defined mass increments into the oligonucleotide molecule, as described herein. Modifications introduced at the phosphodiester bond such as with alpha-thio nucleoside triphosphates, have the advantage that these modifications do not interfere with accurate Watson-Crick base-pairing and additionally allow for the one-step post-synthetic site-specific modification of the complete nucleic acid molecule e.g., via alkylation reactions (see, e.g., Nakamaye et al., Nucl. Acids Res. 23:9947-9959 (1988)). Exemplary mass-modifying functionalities are boron-modified nucleic acids, which can be efficiently incorporated into nucleic acids by polymerases (see, e.g., Porter et al., Biochemistry 34: 11963-11969 (1995); Hasan et al., Nucl. Acids Res. 24:2150-2157 (1996); Li et al. Nucl. Acids Res. 23:4495-4501 (1995)).

Furthermore, the mass-modifying functionality may be added so as to affect chain termination, such as by attaching it to the 3′-position of the sugar ring in the nucleoside triphosphate. For those skilled in the art, it is clear that many combinations can be used in the methods provided herein. In the same way, those skilled in the art will recognize that chain-elongating nucleoside triphosphates also can be mass-modified in a similar fashion with numerous variations and combinations in functionality and attachment positions.

Different mass-modified nucleotides may be used to simultaneously detect a variety of different nucleic acid fragments simultaneously. In one embodiment, mass modifications can be incorporated during the amplification process. In another embodiment, multiplexing of different nucleic acid target gene molecules may be performed by mass modifying one or more nucleic acid target gene molecules, where each different nucleic acid target gene molecule can be differently mass modified, if desired.

Additional mass measurement methods known in the art may be used in the methods of mass measurement, including electrophoretic methods such as gel electrophoresis and capillary electrophoresis, and chromatographic methods including size exclusion chromatography and reverse phase chromatography.

Using methods of mass analysis such as those described herein, information relating to mass of the nucleic acid target gene molecule fragments can be obtained. Additional information of a mass peak that can be obtained from mass measurements include signal to noise ratio of a peak, the peak area (represented, for example, by area under the peak or by peak width at half-height), peak height, peak width, peak area relative to one or more additional mass peaks, peak height relative to one or more additional mass peaks, and peak width relative to one or more additional mass peaks. Such mass peak characteristics may be used in the present methylation identification methods, for example, in a method of identifying the methylation state of a nucleotide locus of a nucleic acid target gene molecule by comparing at least one mass peak characteristic of an amplification fragment with one or more mass peak characteristics of one or more reference nucleic acids.

### Methylation State Identification

Fragment measurements may be used to identify the methylation state of a nucleic acid target gene molecule or to identify the methylation state of a particular nucleotide locus of a nucleic acid target gene molecule. Fragment measurements may be used to identify whether or not a nucleic acid target gene molecule contains one or more methylated or unmethylated nucleotides, such as methylcytosine or cytosine, respectively; to determine the number of methylated or unmethylated nucleotides such as methylcytosine or cytosine, respectively, present in a nucleic acid target gene molecule, to identify whether or not a nucleotide locus, such as a cytosine locus, is methylated or unmethylated in a nucleic acid target gene molecule, to identify the nucleotide locus of a methylated or unmethylated nucleotide, such as methylcytosine or cytosine, respectively, in a nucleic acid target gene molecule; to determine the ratio of methylated nucleic acid target gene molecule relative to unmethylated nucleic acid target gene molecule in a sample, to determine the ratio of methylated nucleotide at a particular nucleotide locus on a nucleic acid target gene molecule relative to unmethylated nucleotide at that locus, and to provide redundant information to further confirm any of the determinations provided herein.

### Additional Methylation Analysis Methods

Various methylation assay procedures are known in the art, and can be used in conjunction with the present invention. These assays allow for determination of the methylation state of one or a plurality of CpG islands within a DNA sequence. Such assays involve, among other techniques, DNA sequencing of bisulfite-treated DNA, PCR (for sequence-specific amplification), Southern blot analysis, use of methylation-sensitive restriction enzymes, etc.

For example, genomic sequencing has been simplified for analysis of DNA methylation patterns and 5-methylcytosine distribution by using bisulfite treatment (Frommer et al., Proc. Natl. Acad. Sci. USA 89:1827-1831, 1992). Additionally, restriction enzyme digestion of PCR products amplified from bisulfite-converted DNA is used, e.g., the method described by Sadri & Hornsby (Nucl. Acids Res. 24:5058-5059, 1996), or COBRA (Combined Bisulfite Restriction Analysis) (Xiong & Laird, Nucleic Acids Res. 25:2532-2534, 1997).

COBRA analysis is a quantitative methylation assay useful for determining DNA methylation levels at specific gene loci in small amounts of genomic DNA (Xiong & Laird, Nucleic Acids Res. 25:2532-2534, 1997). Briefly, restriction enzyme digestion is used to reveal methylation-dependent sequence differences in PCR products of sodium bisulfite-treated DNA. Methylation-dependent sequence differences are first introduced into the genomic DNA by standard bisulfite treatment according to the procedure described by Frommer et al. (Proc. Natl. Acad. Sci. USA 89:1827-1831, 1992). PCR amplification of the bisulfite converted DNA is then performed using primers specific for the interested CpG islands, followed by restriction endonuclease digestion, gel electrophoresis, and detection using specific, labeled hybridization probes. Methylation levels in the original DNA sample are represented by the relative amounts of digested and undigested PCR product in a linearly quantitative fashion across a wide spectrum of DNA methylation levels. In addition, this technique can be reliably applied to DNA obtained from microdissected paraffin-embedded tissue samples. Typical reagents (e.g., as might be found in a typical COBRA-based kit) for COBRA analysis may include, but are not limited to: PCR primers for specific gene (or methylation-altered DNA sequence or CpG island); restriction enzyme and appropriate buffer; gene-hybridization oligo; control hybridization oligo; kinase labeling kit for oligo probe; and radioactive nucleotides. Additionally, bisulfite conversion reagents may include: DNA denaturation buffer; sulfonation buffer; DNA recovery reagents or kits (e.g., precipitation, ultrafiltration, affinity column); desulfonation buffer; and DNA recovery components.

Preferably, assays such as “MethyLight™.” (a fluorescence-based real-time PCR technique) (Eads et al., Cancer Res. 59:2302-2306, 1999), Ms-SNuPE (Methylation-sensitive Single Nucleotide Primer Extension) reactions (Gonzalgo & Jones, Nucleic Acids Res. 25:2529-2531, 1997), methylation-specific PCR (“MSP”; Herman et al., Proc. Natl. Acad. Sci. USA 93:9821-9826, 1996; U.S. Pat. No. 5,786,146), and methylated CpG island amplification (“MCA”; Toyota et al., Cancer Res. 59:2307-12, 1999) are used alone or in combination with other of these methods.

The MethyLight™ assay is a high-throughput quantitative methylation assay that utilizes fluorescence-based real-time PCR (TaqMan®) technology that requires no further manipulations after the PCR step (Eads et al., Cancer Res. 59:2302-2306, 1999). Briefly, the MethyLight™ process begins with a mixed sample of genomic DNA that is converted, in a sodium bisulfite reaction, to a mixed pool of methylation-dependent sequence differences according to standard procedures (the bisulfite process converts unmethylated cytosine residues to uracil). Fluorescence-based PCR is then performed either in an “unbiased” (with primers that do not overlap known CpG methylation sites) PCR reaction, or in a “biased” (with PCR primers that overlap known CpG dinucleotides) reaction. Sequence discrimination can occur either at the level of the amplification process or at the level of the fluorescence detection process, or both.

The MethyLight™ assay may be used as a quantitative test for methylation patterns in the genomic DNA sample, wherein sequence discrimination occurs at the level of probe hybridization. In this quantitative version, the PCR reaction provides for unbiased amplification in the presence of a fluorescent probe that overlaps a particular putative methylation site. An unbiased control for the amount of input DNA is provided by a reaction in which neither the primers, nor the probe overlie any CpG dinucleotides. Alternatively, a qualitative test for genomic methylation is achieved by probing of the biased PCR pool with either control oligonucleotides that do not “cover” known methylation sites (a fluorescence-based version of the “MSP” technique), or with oligonucleotides covering potential methylation sites.

The MethyLight™ process can by used with a “TaqMan®” probe in the amplification process. For example, double-stranded genomic DNA is treated with sodium bisulfite and subjected to one of two sets of PCR reactions using TaqMan® probes; e.g., with either biased primers and TaqMan® probe, or unbiased primers and TaqMan® probe. The TaqMan® probe is dual-labeled with fluorescent “reporter” and “quencher” molecules, and is designed to be specific for a relatively high GC content region so that it melts out at about 10.degree. C. higher temperature in the PCR cycle than the forward or reverse primers. This allows the TaqMan® probe to remain fully hybridized during the PCR annealing/extension step. As the Taq polymerase enzymatically synthesizes a new strand during PCR, it will eventually reach the annealed TaqMan® probe. The Taq polymerase 5′ to 3′ endonuclease activity will then displace the TaqMan® probe by digesting it to release the fluorescent reporter molecule for quantitative detection of its now unquenched signal using a real-time fluorescent detection system.

Typical reagents (e.g., as might be found in a typical MethyLight™-based kit) for MethyLight™ analysis may include, but are not limited to: PCR primers for specific gene (or methylation-altered DNA sequence or CpG island); TaqMan® probes; optimized PCR buffers and deoxynucleotides; and Taq polymerase.

Ms-SNuPE. The Ms-SNuPE technique is a quantitative method for assessing methylation differences at specific CpG sites based on bisulfite treatment of DNA, followed by single-nucleotide primer extension (Gonzalgo & Jones, Nucleic Acids Res. 25:2529-2531, 1997).

Briefly, genomic DNA is reacted with sodium bisulfite to convert unmethylated cytosine to uracil while leaving 5-methylcytosine unchanged. Amplification of the desired target sequence is then performed using PCR primers specific for bisulfite-converted DNA, and the resulting product is isolated and used as a template for methylation analysis at the CpG site(s) of interest.

Small amounts of DNA can be analyzed (e.g., microdissected pathology sections), and it avoids utilization of restriction enzymes for determining the methylation status at CpG sites.

Typical reagents (e.g., as might be found in a typical Ms-SNuPE-based kit) for Ms-SNuPE analysis may include, but are not limited to: PCR primers for specific gene (or methylation-altered DNA sequence or CpG island); optimized PCR buffers and deoxynucleotides; gel extraction kit; positive control primers; Ms-SNuPE primers for specific gene; reaction buffer (for the Ms-SNuPE reaction); and radioactive nucleotides. Additionally, bisulfite conversion reagents may include: DNA denaturation buffer; sulfonation buffer; DNA recovery regents or kit (e.g., precipitation, ultrafiltration, affinity column); desulfonation buffer; and DNA recovery components.

MSP (methylation-specific PCR) allows for assessing the methylation status of virtually any group of CpG sites within a CpG island, independent of the use of methylation-sensitive restriction enzymes (Herman et al. Proc. Nat. Acad. Sci. USA 93:9821-9826, 1996; U.S. Pat. No. 5,786,146). Briefly, DNA is modified by sodium bisulfite converting all unmethylated, but not methylated cytosines to uracil, and subsequently amplified with primers specific for methylated versus unmethylated DNA. MSP requires only small quantities of DNA, is sensitive to 0.1% methylated alleles of a given CpG island locus, and can be performed on DNA extracted from paraffin-embedded samples. Typical reagents (e.g., as might be found in a typical MSP-based kit) for MSP analysis may include, but are not limited to: methylated and unmethylated PCR primers for specific gene (or methylation-altered DNA sequence or CpG island), optimized PCR buffers and deoxynucleotides, and specific probes.

The MCA technique is a method that can be used to screen for altered methylation patterns in genomic DNA, and to isolate specific sequences associated with these changes (Toyota et al., Cancer Res. 59:2307-12, 1999). Briefly, restriction enzymes with different sensitivities to cytosine methylation in their recognition sites are used to digest genomic DNAs from primary tumors, cell lines, and normal tissues prior to arbitrarily primed PCR amplification. Fragments that show differential methylation are cloned and sequenced after resolving the PCR products on high-resolution polyacrylamide gels. The cloned fragments are then used as probes for Southern analysis to confirm differential methylation of these regions. Typical reagents (e.g., as might be found in a typical MCA-based kit) for MCA analysis may include, but are not limited to: PCR primers for arbitrary priming Genomic DNA; PCR buffers and nucleotides, restriction enzymes and appropriate buffers; gene-hybridization oligos or probes; control hybridization oligos or probes.

Another method for analyzing methylation sites is a primer extension assay, including an optimized PCR amplification reaction that produces amplified targets for subsequent primer extension genotyping analysis using mass spectrometry. The assay can also be done in multiplex. This method (particularly as it relates to genotyping single nucleotide polymorphisms) is described in detail in PCT publication WO05012578A1 and US publication US20050079521A1. For methylation analysis, the assay can be adopted to detect bisulfite introduced methylation dependent C to T sequence changes. These methods are particularly useful for performing multiplexed amplification reactions and multiplexed primer extension reactions (e.g., multiplexed homogeneous primer mass extension (hME) assays) in a single well to further increase the throughput and reduce the cost per reaction for primer extension reactions.

Four additional methods for DNA methylation analysis include restriction landmark genomic scanning (RLGS, Costello et al., 2000), methylation-sensitive-representational difference analysis (MS-RDA), methylation-specific AP-PCR (MS-AP-PCR) and methyl-CpG binding domain column/segregation of partly melted molecules (MBD/SPM).

Additional methylation analysis methods that may be used in conjunction with the present invention are described in the following: Laird, P. W. Nature Reviews Cancer 3, 253-266 (2003); Biotechniques; Uhlmann, K. et al. Electrophoresis 23:4072-4079 (2002)—PyroMeth; Colella et al. Biotechniques. 2003 July; 35(1):146-50; Dupont J M, Tost J, Jammes H, and Gut I G. Anal Biochem, October 2004; 333(1): 119-27; Tooke N and Pettersson M. IVDT. November 2004; 41; and the following published patents and patent applications: WO03080863A1, WO03057909A2, US2005/0153347, US20050009059A1, US20050069879A1, US20050064428A1, US20050064406A1, WO02086163C1, US20050019762A1, U.S. Pat. No. 6,884,586, WO04013284A2, US20050153316A1 and WO05040399A2; and U.S. patent application Ser. No. 10/888,359 filed Jul. 9, 2004, entitled “Methods and Compositions for Phenotype Identification Based on Nucleic Acid Methylation;” International Patent Application No. PCT/US2005/009929 filed Mar. 24, 2005, U.S. patent application Ser. No. 11/089,805 filed Mar. 24, 2005 and U.S. Provisional Patent Application No. 60/556,632 filed Mar. 26, 2004, each entitled “Base Specific Cleavage Of Methylation-Specific Amplification Products In Combination With Mass Analysis;” and U.S. patent application Ser. No. 10/272,665 filed Oct. 15, 2002, entitled “Methods For Generating Databases And Databases For Identifying Polymorphic Genetic Markers.”

### Disease-Related Discovery

In one embodiment, presence or absence of one or more methylated or unmethylated nucleotides may be identified as indicative of a particular disease outcome associated with methylated or unmethylated DNA. In another embodiment, presence or absence of one or more methylated or unmethylated nucleotides may be identified as indicative of a normal, healthy or disease free state. In another embodiment, an abnormal ratio of methylated nucleic acid target gene molecules relative to unmethylated nucleic acid target gene molecules in a sample may be indicative of a particular disease outcome associated with methylated or unmethylated DNA. For example, a relatively high number or a relatively low number of methylated nucleic acid target gene molecules compared to the relative amount in a normal individual may be indicative of a good prognosis disease state associated with methylated or unmethylated DNA. In another embodiment, an abnormal ratio of methylated nucleotide at a nucleotide locus relative to unmethylated nucleotide at a nucleotide locus in a nucleic acid target gene molecule can be indicative of a poor prognosis disease state associated with methylated or unmethylated DNA. For example, a relatively high number or a relatively low number of methylated nucleotide loci compared to the relative amount in a normal individual can be indicative of a poor prognosis disease state associated with methylated or unmethylated DNA.

### Disease-Related Analysis

Increased or decreased levels of methylation have been associated with a variety of diseases. Methylation or lack of methylation at defined positions can be associated with a disease or a disease outcome. The methods disclosed herein can be used in methods of determining the propensity of a subject to disease, diagnosing a disease, prognosing a disease and determining a treatment regimen for a subject having a disease.

Diseases associated with a modification of the methylation of one or more nucleotides include, for example: leukemia (Aoki E. et al., “Methylation status of the p151NK4B gene in hematopoietic progenitors and peripheral blood cells in myelodysplastic syndromes”, Leukemia 14(4):586-593 (2000); Nosaka, K. et al., “Increasing methylation of the CDKN2A gene is associated with the progression of adult T-cell leukemia”, Cancer Res. 60(4):1043-1048 (2000); Asimakopoulos F A et al., “ABL 1 methylation is a distinct molecular event associated with clonal evolution of chronic myeloid leukemia” Blood 94(7):2452-2460 (1999); Fajkusova L. et al., “Detailed Mapping of Methylcytosine Positions at the CpG Island Surrounding the Pa Promoter at the bcr-abl Locus in CML Patients and in Two Cell Lines, K562 and BV173” Blood Cells Mol. Dis. 26(3):193-204 (2000); Litz C. E. et al., “Methylation status of the major breakpoint cluster region in Philadelphia chromosome negative leukemias” Leukemia 6(1):35-41 (1992))

The methylation state of a variety of nucleotide loci and/or nucleic acid regions are known to be correlated with a disease, disease outcome, and success of treatment of a disease, and also may be used to distinguish disease types that are difficult to distinguish according to the symptoms, histologic samples or blood or serum samples. For example, CpG island methylator indicator phenotype (CIMP) is present in some types of ovarian carcinomas, but not in other types (Strathdee, et al., Am. J. Pathol. 158:1121-1127 (2001)). In another example, methylation may be used to distinguish between a carcinoid tumor and a pancreatic endocrine tumor, which may have different expected outcomes and disease treatment regimens (Chan et al., Oncogene 22:924-934 (2003)). In another example, H. pylori dependent gastric mucosa associated lymphoid tissue (MALT) lymphomas are characterized as having several methylated nucleic acid regions, while those nucleic acid regions in H. pylori independent MALT lymphomas are not methylated Kaneko et al., Gut 52:641-646 (2003)). Similar relationships with disease, disease outcome and disease treatment have been correlated with hypomethylation or unmethylated nucleic acid regions or unmethylated nucleotide loci.

Methods related to the disease state of a subject may be performed by collecting a sample from a subject, treating the sample with a reagent that modifies a nucleic acid target gene molecule sequence as a function of the methylation state of the nucleic acid target gene molecule, subjecting the sample to methylation specific amplification, then detecting one or more fragments that are associated with a disease outcome (measured as survivability). In another embodiment, the fragments are detected by measuring the mass of the nucleic acid target gene molecule or nucleic acid target gene molecule fragments. Detection of a nucleic acid target gene molecule or nucleic acid target gene molecule fragment can identify the methylation state of a nucleic acid target gene molecule or the methylation state of one or more nucleotide loci of a nucleic acid target gene molecule. Identification of the methylation state of a nucleic acid target gene molecule or the methylation state of one or more nucleotide loci of a nucleic acid target gene molecule can indicate the propensity of the subject toward one or more diseases, the disease state of a subject, likelihood of survival or an appropriate or inappropriate course of disease treatment or management for a subject.

### Applications of Prognostic and Diagnostic Results to Pharmacogenomic Methods

Pharmacogenomics is a discipline that involves tailoring a treatment for a subject according to the subject's genetic profile (e.g., genotype, methylation state or characteristic methylation state). For example, based upon the outcome of a prognostic test described herein, a clinician or physician may target pertinent information and preventative or therapeutic treatments to a subject who would benefit by the information or treatment and avoid directing such information and treatments to a subject who would not be benefited (e.g., the treatment has no therapeutic effect, the subject experiences adverse side effects, and/or the treatment poses unnecessary risks given the prognosis).

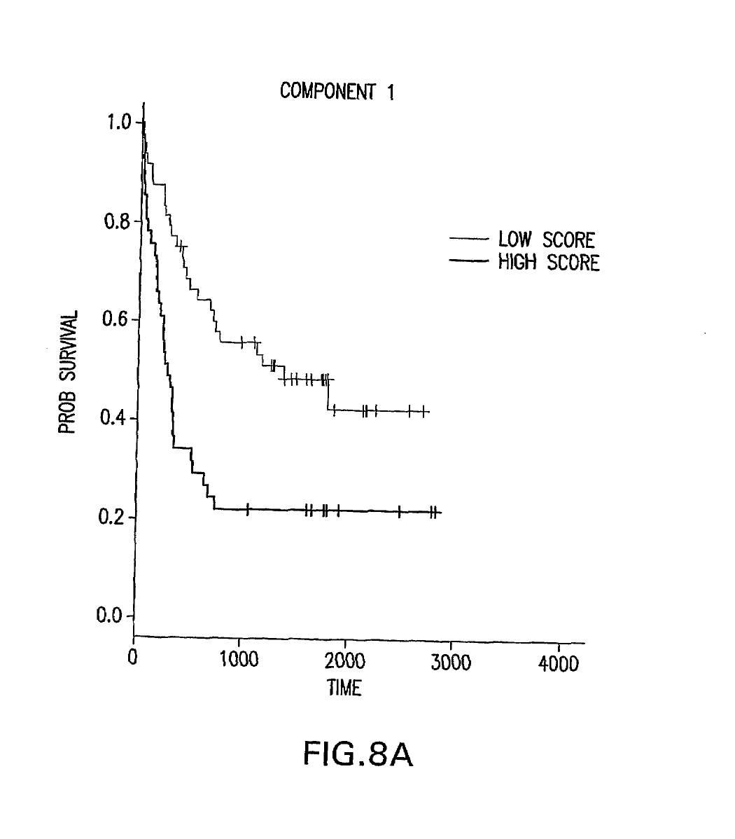

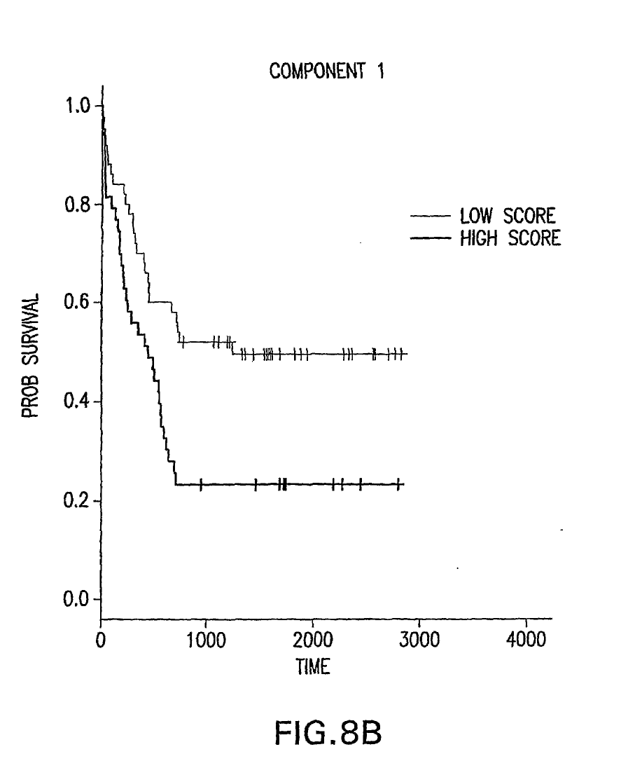

The following is an example of a pharmacogenomic embodiment. A particular treatment regimen can exert a differential effect depending upon the subject's characteristic methylation state. Where a candidate therapeutic response is correlated with a given methylation state (e.g., high methylation score in FIGS. 8A-C), a therapeutic typically would not be administered to a subject determined to have a methylation state that correlates with a poor response, and conversely may be administered to a subject determined to have a methylation state that correlates with a positive response. In another example, where a candidate therapeutic is significantly toxic (e.g., a chemotherapeutic agent) when administered to subjects, a subject with a good prognosis may be willing to endure the adverse effects and risks associated with the toxic therapeutic more so than a patient with a poor prognosis that is unlikely to survive regardless of the therapeutic administered.

The methods described herein are applicable to pharmacogenomic methods for preventing, alleviating or treating AML. For example, a nucleic acid sample from an individual may be subjected to a prognostic test described herein. Where a methylation state or characteristic methylation state that is predictive of AML outcome is identified in a subject, information for preventing or treating AML and/or one or more AML treatment regimens then may be prescribed to that subject.

In certain embodiments, a treatment or preventative regimen is specifically prescribed and/or administered to individuals who will most benefit from it based upon their likelihood of survival assessed by the methods described herein. Thus, provided are methods for determining a prognosis for AML patients and then prescribing a therapeutic or preventative regimen to individuals according to their prognosis. Thus, certain embodiments are directed to methods for determining the appropriate therapeutic regimen for a subject, which comprises: treating a nucleic acid sample with a reagent that modifies unmethylated cytosine to produce uracil; amplifying a nucleic acid target gene region using at least one primer that hybridizes to a strand of said nucleic acid target gene region producing amplified nucleic acids; determining the characteristic methylation state of said nucleic acid target gene region by base specific cleavage and identification of methylation sites of said amplified nucleic acids; comparing the ratio of methylated cytosine to unmethylated cytosine for each of said methylation sites of said characteristic methylation state of said sample to the ratio of methylated cytosine to unmethylated cytosine for each of said methylation sites of a subject or group of subjects having a known disease outcome thereby predicting the probability of said subject's survival; wherein a subject with a poor prognosis is administered an poor prognosis treatment regimen and a subject with a good prognosis is administered a good prognosis treatment regimen. In these methods, predisposition results may be utilized in combination with other test results or risk factors to diagnose hematology-related cancers, such as AML. Risk factors for AML include heredity, exposure to radiation, chemical and other occupational hazards, and antineoplastic drugs which are further described herein.

Pharmacogenomics methods also may be used to analyze and predict a response to an AML treatment or a drug. For example, if pharmacogenomics analysis indicates a likelihood that an individual will respond positively to an AML treatment with a particular drug or combination of drugs, the drug(s) may be administered to the individual. Conversely, if the analysis indicates that an individual is likely to respond negatively to treatment with a particular drug or combination of drugs, an alternative course of treatment may be prescribed. A negative response may be defined as either the absence of an efficacious response or the presence of toxic side effects. The response to a therapeutic treatment can be predicted in a background study in which the methylation state of subjects in any of the following populations is determined: a population that responds favorably to a treatment regimen, a population that does not respond significantly to a treatment regimen, and a population that responds adversely to a treatment regiment (e.g., exhibits one or more side effects). These populations are provided as examples and other populations and subpopulations may be analyzed.

The tests described herein also are applicable to clinical drug trials. A subject's prognosis may be determined using the methods described herein. Thereafter, subjects with a poor prognosis may choose to participate in clinical trials that may increase their probability of survival but have unknown or high-risk side effects; whereas subjects with a good prognosis may choose to undergo treatments that have higher success rates but expose the subject to adverse side effects. Alternatively, subjects with a good prognosis might choose to enroll in a clinical trial for a treatment which decreases a risk of relapse or a clinical trial with known or low-risk side effects.

Also provided herein is a method of partnering between a diagnostic/prognostic testing provider and a provider of a consumable product, which comprises: (a) the diagnostic/prognostic testing provider determines a subject's prognosis; (b) the diagnostic/prognostic testing provider forwards information to the subject about a particular product which may be obtained and consumed or applied by the subject given their prognosis; and (c) the provider of a consumable product forwards to the diagnostic test provider a fee every time the diagnostic/prognostic test provider forwards information to the subject as set forth in step (b) above.

### Combinations and Kits

In another embodiment, there are provided prognostic or diagnostic systems, typically in combination or kit form, containing a reagent that modifies one or more nucleotides of the nucleic acid target gene molecule as a function of the methylation state of the nucleic acid target gene molecule, such as bisulfite; one or more methylation specific primers for specifically hybridizing to a reagent-treated nucleic acid target gene molecule, such as one or more methylation specific PCR primers; and one or more compounds for fragmenting amplified nucleic acid target gene molecule, such as RNases, including RNase A or RNase T1. A kit also may include the appropriate buffers and solutions for performing the methylation identification methods described herein. For example, a kit can include a glass vial used to contain milligram quantities of a primer or enzyme. A kit also may include substrates, supports or containers for performing the methylation identification methods, including vials or tubes, or a mass spectrometry substrate such as a Sequenom SpectroCHIP substrate.

### EXAMPLES

The following Examples describe a novel technique that uses base-specific cleavage of amplification products and Matrix-Assisted Laser Desorption/Ionization Time-Of-Flight Mass Spectrometry (MALDI-TOF MS) to perform large scale quantitative DNA methylation analysis across a set of candidate genes (n=147). This method led to the identification of clinically relevant AML subclasses, while highlighting methylated genes of potential pathogenic relevance. Also described is a methylation-based outcome predictor derived from AML-associated promoter methylation patterns that provide a basis for improved outcome prediction in AML.

### Example 1

### Bisulfite Treatment of a Nucleic Acid Target Gene Region

Bisulfite treatment of genomic DNA was performed with a commercial kit from Zymo Research Corporation (Orange, Calif.) that combines bisulfite conversion and DNA clean up. The kit follows a protocol from Paulin, R. et al. in Nucleic Acids Res. 26:5009-5010, 1998. Briefly, in this protocol 2 μg of genomic DNA is digested with a restriction endonuclease (EcoRI), then denatured by the addition of 3 M sodium hydroxide and incubated for 15 min at 37° C. A 6.24 M urea/2 M sodium metabisulfite (4 M bisulfite) solution is prepared and added with 10 mM hydroquinone to the denatured DNA. The corresponding final concentrations are 5.36 M, 3.44 M and 0.5 mM respectively. The reaction is performed in a 0.5 ml tube overlaid with mineral oil. This reaction mix is repeatedly heated between 55° C. for 15 min and 95° C. for 30 s in a PCR machine (MJ Tetrad) for 20 cycles. DNA purification was done using the commercially available GENECLEAN kit from Q-biogene.

### Example 2

### PCR and In Vitro Transcription of a Nucleic Acid Target Gene Region

The IGF2/H19 gene region (Human Genome Chromosome 11:1,983,678-1,984,097) serves as an exemplary gene to demonstrate the effectiveness and feasibility of the methylation analysis methods disclosed herein. The IGF2/H19 region provides an ideal test case because of its hemi-methylated status. In a hemi-methylated region, the paternal allele is usually silenced by methylation, which results in an ideal 50/50 ratio. The presence of an expected 50/50 ratio validates the approach. As the following Examples demonstrate, this is in fact the case, and the methods used to analyze IGF2/H19 were applied to the AML target genes disclosed herein.

IGF2/H19 was PCR-amplified from bisulfite treated human genomic DNA using primers that incorporate the T7 [5′-CAG TAA TAC GAC TCA CTA TAG GGA GA] (SEQ ID NO: 359) promoter sequence. Two sets of primers were designed to incorporate the T7 promoter sequence either to the forward (5′-CAG TAA TAC GAC TCA CTA TAG GGA GAA GGC TGT TAG TTT TTA TTT TAT TTT TAA T-3′ (SEQ ID NO: 369); 5′-AGG AAG AGA GAA CCA CTA TCT CCC CTC AAA AAA-3′) (SEQ ID NO: 361) or to the reverse (5′-AGG AAG AGA GGT TAG TTT TTA TTT TAT TTT TAA T-3′ (SEQ ID NO: 362); 5′-CAG TAA TAC GAC TCA CTA TAG GGA GAA GGC TAA CCA CTA TCT CCC CTC AAA AAA-3′) (SEQ ID NO: 363) strand. Alternatively the derived PCR product was cloned into a pGEM-T vector system (Promega, Madison, Wis.) and re-amplified from the cloned DNA. The PCR reactions were carried out in a total volume of 5 μl using 1 μmol of each primer, 40 μM dNTP, 0.1 U Hot Star Taq DNA polymerase (Qiagen, Valencia, Calif.), 1.5 mM MgCl₂ and buffer supplied with the enzyme (final concentration 1×). The reaction mix was pre-activated for 15 min at 95° C. The reactions were amplified in 45 cycles of 95° C. for 20 s, 62° C. for 30 s and 72° C. for 30 s followed by 72° C. for 3 min. Unincorporated dNTPs were dephosphorylated by adding 1.7 ul H2O and 0.3 U Shrimp Alkaline Phosphatase. The reaction was incubated at 37° C. for 20 min and SAP was then heat-inactivated for 10 minutes at 85° C.

Typically, two microliters of the PCR reaction were directly used as template in a 4 μl transcription reaction. Twenty units of T7 R&DNA polymerase (Epicentre, Madison, Wis.) were used to incorporate either dCTP or dTTP in the transcripts. Ribonucleotides were used at 1 mM and the dNTP substrate at 2.5 mM; other components in the reaction were as recommended by the supplier. Following the in vitro transcription, RNase A (SEQUENOM, San Diego) was added to cleave the in vitro transcript. The mixture was then further diluted with H₂O to a final volume of 27 μl. Conditioning of the phosphate backbone prior to MALDI-TOF MS was achieved by the addition of 6 mg CLEAN Resin (SEQUENOM Inc., San Diego, Calif.).

### Example 3

### Mass Spectral Measurements of Transcribed Nucleic Acid Target Gene Region

Conditioning of the phosphate backbone was achieved by the addition of 6 mg CLEAN Resin (Sequenom Inc., San Diego, Calif.) to the transcription sample. A 15 nl aliquot of the cleavage reaction was robotically dispensed onto a silicon chip preloaded with matrix (SpectroCHIP; Sequenom Inc., San Diego, Calif. Mass spectra were collected using a MassARRAY mass spectrometer (Bruker-SEQUENOM). Spectra were analyzed using proprietary peak picking and spectra interpretation tools (Little, et al. Nat Med 3:1413-6 (1997)).

### Example 4

### Identification of Methylation Sites in IGF2/H19

The difference in the mass spectra results from a C-specific cleavage reaction of the forward transcript may be seen in FIG. 1. The mass spectrum derived from the methylated template shows signals corresponding to the expected methylation sites. In this spectra each mass signal represents at least two CpG sites (cleavage at the beginning of the fragment and at the end) and two cleavage products therefore represent each methylated CpG site. The non-methylated template creates a mass spectrum that is devoid of any sequence/methylation associated signals. FIG. 1 displays mass signals generated by cytosine specific cleavage of the forward transcript of the IGF2/H19 region (upper spectral analysis is the methylated template; lower spectral analysis is the non-methylated template). Methylation of the target sequence results in the generation of rCTP-containing transcripts; every methylated CpG is represented in the transcript by a cleavage site. Each of the cleavage products is labeled with a number, which indicates the CpG position in the template. These numbers can be cross-referenced with the cleavage products listed in Tables 2 and 3. The non-methylated target sequence does not contain cytosine and therefore does not contain cleavage sites. Mass signals are labeled with letters and the corresponding explanations are listed in FIG. 1(B). A full list of expected cleavage products illustrates the predicted difference between methylated and non-methylated template. Predicted mass signals 12 and 13 are not found in the experimental spectrum, because the corresponding CpGs 23 and 24 are not methylated which results in concatenation of fragment 5167 and 12616 in a much larger fragment that can not be detected.

The below tables show the cleavage products of mass signals generated by cytosine specific cleavage of the forward transcript of IGF2/H19 in both the methylated (Table 2) and non-methylated (Table 3) transcript sequences.

TABLE 2

| | | | | |
|---|---|---|---|---|
| | CpG | | | |
| Molecular | island | Cleavage | | |
| Mass in Da | position | product type | SEQ ID NO: | Cleavage product composition and origin |

| | | | | |
|---|---|---|---|---|
| 653.41 | OOMR | MAIN | | 5OH-AC-3p @447 |
| 669.41 | OOMR | MAIN | | 5OH-GC-3p @227; 5OH-GC-3p @169; 5OH-GC-3p @116; |
| | | | | 5OH-GC-3p @63 |
| 932.60 | OOMR | MAIN | | 5OH-TTC-3p @431 |
| 1236.8 | OOMR | MAIN | | 5OH-TTTC-3p @434 |
| 1277.81 | OOMR | ANCH | | 5OH-GTTC-3p @59 |
| 1535.07 | 1 | LAST | | 5OH-TAAAT-3OH @450 |
| 1648.03 | 2 | MAIN | | 5OH-GAGTC-3p @65 |
| 1993.24 | 3 | MAIN | | 5OH-GGTGAC-3p @171 |
| 2215.41 | 4 | ANCH | | 5OH-GTTTATC-3p @35 |
| 2306.45 | 5 | MAIN | | 5OH-GGAAATC-3p @162 |
| 3260.05 | 6 | ANCH | 370 | 5OH-GGTAATATGC-3p @83 |
| 3301.07 | 7 | MAIN | 371 | 5OH-GAATGGGATC-3p @118 |
| 3623.03 | 8 | ANCH | 372 | 5PPP-GGGAGAAGGC-3p @0 |
| 3893.46 | 9 | ANCH | 373 | 5OH-GAGTATAAGTTC-3p @177 |
| 3941.46 | 10 | ANCH | 374 | 5OH-GGGGTGTTTAGC-3p @128 |
| 4197.66 | 11 | ANCH | 375 | 5OH-GTAAGTATAGTTC-3p @70 |
| 4574.86 | 12* | ANCH | 376 | 5OH-GTGAGGTTTGAGTC-3p @294 |
| 5167.26 | 13* | ANCH | 377 | 5OH-GAGTTTTTAGGGATTC-3p @343 |
| 5512.47 | 14 | ANCH | 378 | 5OH-GTTTGTTAGTAGAGTGC-3p @42 |
| 5832.66 | 15 | ANCH | 379 | 5OH-GTTTATTAGGGGGTTTGC-3p @229 |
| 6211.9 | 16 | MAIN | 380 | 5OH-GGTTAATTGGATGGGAATC-3p @189 |
| 6234.88 | 17 | MAIN | 381 | 5OH-GGTTTGGGGGGTTGGTATC-3p @208 |
| 7106.46 | 18 | MAIN | 382 | 5OH-GGTTGTGGGGATTTTGTTTTGC-3p @140 |
| 7494.71 | 19 | MAIN | 383 | 5OH-GGTTTTTAGATAGGAAAGTGGTC-3p @93 |
| 8192.12 | 20 | MAIN | 384 | 5OH-TGGGTTTGGGAGAGTTTGTGAGGTC-3p @10 |
| 9870.16 | OOMR | MAIN | 385 | 5OH-GTTGGTAGGTAGGGAGTAGTAGGTATGGTC-3p @398 |
| 11366.1 | OOMR | MAIN | 386 | 5OH-GTGATGGTGGTAGGAAGGGGTTTTTTGTGTTATTC-3p @308 |
| 12616.9 | OOMR | MAIN | 387 | 5OH-GTAGTTGGTTTTTAGTTATGTGTAAAGTATGTGTAGGGC-3p @359 |
| 14763.4 | OOMR | MAIN | 388 | 5OH-GGTATTTTTTTTTGTTTTTTAGTATTTTATTTTTATTTTTTAGGAAC-3p @247 |

TABLE 3

| | | | | |
|---|---|---|---|---|
| | CpG | | Cleavage | |
| Molecular | island | | product | |
| Mass in Da | position | SEQ ID NO: | type | Cleavage product composition and origin |

| | | | | |
|---|---|---|---|---|
| 324.208 | OOMR | | MAIN | 5OH-C-3p @449; 5OH-C-3p @430 |
| 524.192 | OOMR | | ACYC | 5PPP-G-3OH @0 |
| 653.417 | OOMR | | MAIN | 5OH-AC-3p @447 |
| 869.401 | OOMR | | ACYC | 5PPP-GG-3OH @0 |
| 932.601 | OOMR | | MAIN | 5OH-TTC-3p @431 |
| 1214.61 | OOMR | | ACYC | 5PPP-GGG-3OH @0 |
| 1236.8 | OOMR | | MAIN | 5OH-TTTC-3p @434 |
| 1889.03 | A | 389 | DBLC | 5PPP-GGGAGAAGGC-3p @0 |
| 2547.45 | B | | ACYC | 5PPP-GGGAGAA-3OH @0 |
| 2889.83 | C | | MAIN | 5OH-TATAGTGTC-3p @438 derived from PCR primer tag |
| 2892.66 | C | | ACYC | 5PPP-GGGAGAAG-3OH @0 |
| 3237.87 | D | | ACYC | 5PPP-GGGAGAAGG-3OH @0 |
| 3623.03 | E | 390 | MAIN | 5PPP-GGGAGAAGGC-3p @0 derived from PCR primer tag |
| 135810 | OOMR | 391 | MAIN | 5OH-TGGGTTTGGGAGAGTTTGTGAGGTTGTTTATTGTTTGTTAGT |
| | | | | AGAGTGTGTTTGTGAGTTGTAAGTATAGTTTGGTAATATGTGGTT |
| | | | | TTTAGATAGGAAAGTGGTTGTGAATGGGATTGGGGTGTTTAGTGG |
| | | | | TTGTGGGGATTTTGTTTTGTGGAAATTGTGGTGATGAGTATAAGTT |
| | | | | TGGTTAATTGGATGGGAATTGGTTTGGGGGGTTGGTATTGTGTTTA |
| | | | | TTAGGGGGTTTGTGGTATTTTTTTTTGTTTTTTAGTATTTTATTTTTA |
| | | | | TTTTTTAGGAATGTGAGGTTTGAGTTGTGATGGTGGTAGGAAGGGG |
| | | | | TTTTTTGTGTTATTTGAGTTTTTAGGGATTTGTAGTTGGTTTTTAGTT |
| | | | | ATGTGTAAAGTATGTGTAGGGTGTTGGTAGGTAGGGAGTAGTAGGT |
| | | | | ATGGTAGC-3p @10 |

Cleavage product characterization legend:
MAIN = regular cleavage product
OOMR = out of mass range (molecular mass either too low or too high to be detected within the automated data acquisition)
DBLC = double charged molecular ion species (at half mass of parent molecular ion)
ACYC = Abortive cycling (incomplete transcription products generated during the first 10 nt of transcription)

All masses below 1300 Da cannot be detected reliably in the chosen mass window. The mass signal labeled A is a doubly charged molecular ion E. Mass signals labeled B and D represent so called abortive cycling products. Abortive cycling is the premature” termination during the transcribtioon process while the polymerase has still formed the initiation complex and has not yet reached the more stable elongation complex. During that phase the transcribtin might occasionally be terminated without generating a full length transcribt. Mass signals labeled C and E are expected main signals generated by cleavage of the transcription product.

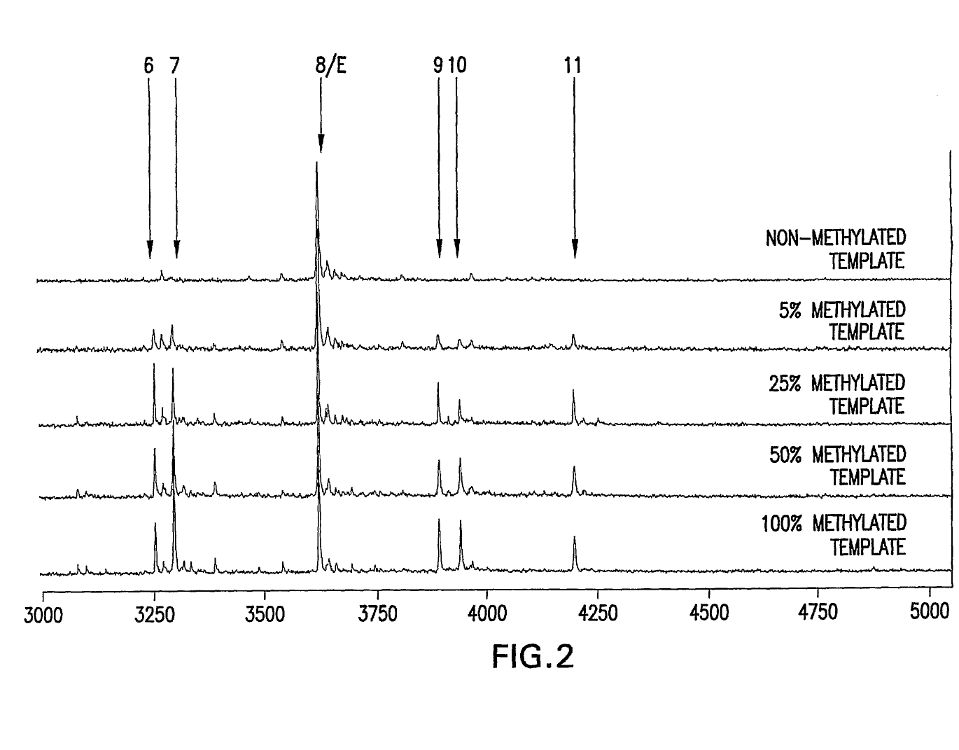

The reactions described above provide ideal mass signal patterns that are well suited to identify methylation in mixtures that contain methylated DNA in a fraction as low as 5%, without selective PCR amplification. FIG. 2 is an overlay of mass signal patterns generated by cytosine specific cleavage of the forward transcript of the IGF2/H19 region. In the depicted case, the template used for PCR amplification consisted of a mixture of methylated and non-methylated DNA. Mass spectra reveal increasing signal intensity of cleavage products with increasing amount of methylated template DNA. Methylation specific mass signals can be detected in mixtures containing as little as 5% methylated DNA.

### Example 5

### Statistical Methods

Base-specific cleavage reactions also can be used in determination of methylation ratios. For example, methylation induced C/T changes on the forward strand are represented as G/A changes on the complementary strand. These changes lead to a mass shift of 16Da (G/A mass shift) or multiples thereof, when multiple CpGs are enclosed in one cleavage product. In reactions where methylation results in a mass shift of nucleic acid target gene molecule fragments, one fragment represents the methylated template and a second fragment represents the non-methylated template. The intensities of the measured masses of these fragments can be compared to determine the ratio of methylated vs. non-methylated nucleic acid target gene molecules. Also, the base composition of the measured fragments differs only by one or a few nucleotides, which assures equal desorption and ionization behavior during MALDI-TOF measurement. Methods for intensity estimation of mass measurements such as “area-under the peak” and “signal to noise” can yield similar results. Depending on the sequence of the nucleic acid target gene molecule, multiple signal pairs can be used in determining the ratio between signal intensities. This information can be used to assess the degree of methylation for each CpG site independently, or, if all CpG sites are methylated approximately to the same degree, to average the methylation content over the complete target region. A direct correlation between signal intensity ratios and the ratio of the deployed DNAs can be determined for ranges of 10%-90% of methylated template. If the ratio between methylated and non-methylated template is below 10% or exceeds 90%, the signals that represent the lower amount of template can still be detected, but the quantitation can be subject to higher error.

All statistical analysis was carried out using the R statistical environment, which is described at the following URL: http://www.R-project.org (R Development Core Team, (2003) R Foundation for Statistical Computing (ISBN) 3-900051-07-0). The “gregmisc” package was used for two-dimensional clustering, the “hclust” package was used for hierarchical cluster analysis, the “survival” package was used for Cox regression analysis. The Kaplan Meier estimates and the “superpc” package (Bair and Tibshirani, PloS Biol 2:E108 (2004)) was used for supervised principle components analysis.

Relative methylation was compared between long and reduced survival groups using the Wilcoxon signed-rank test, a non-parametric counterpart of the paired t-test. The two-way hierarchical cluster analysis clustered samples and CpG units based on pair-wise Euclidean distances and the complete linkage clustering algorithm (Ripley, Pattern Recognition and Neural Networks, Cambridge (1996)). This was carried out using the heatmap.2 function of the “gregmisc” package using the R statistical environment.

### Example 6

### Methylation Ratio Analysis

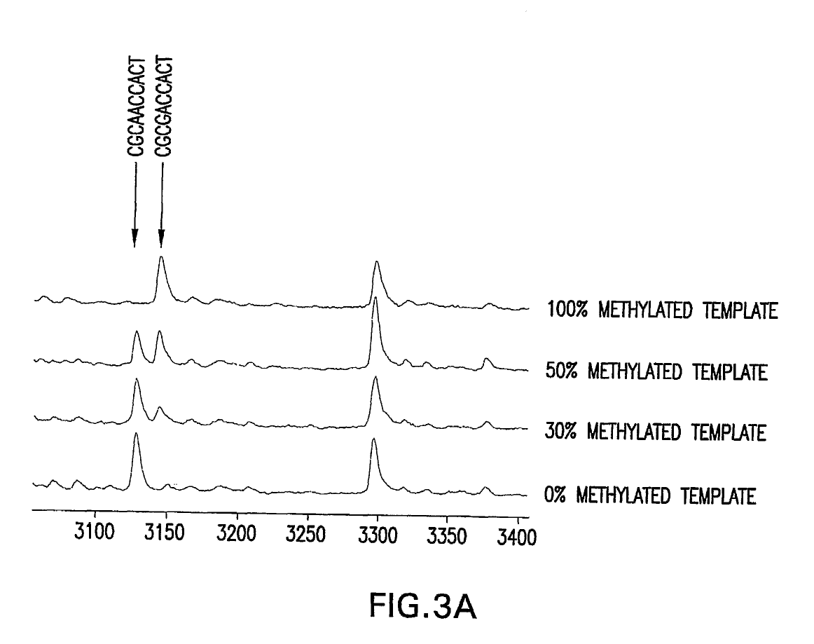

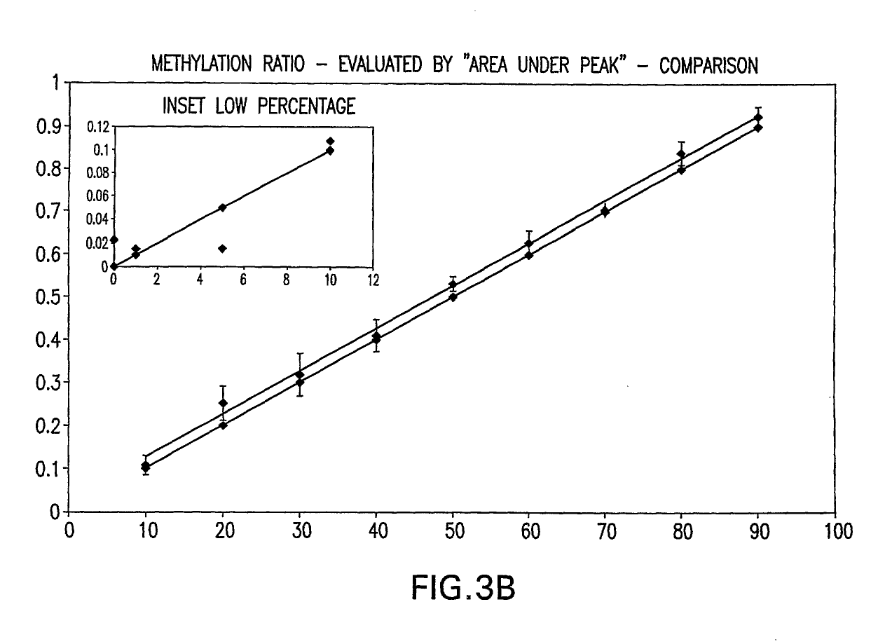

Determination of methylation ratios is enabled by a different base-specific cleavage reaction. Methylation induced C/T changes on the forward strand are represented as G/A changes on the reverse strand. Since cleavage schemes were restricted to C- and T-specific cleavage, methylation events led to a mass shift of 16Da (G/A mass shift) or a multitude thereof when multiple CpGs are enclosed in one cleavage product. The signal pair shown in FIG. 3 demonstrates this. FIG. 3 is an overlay of mass spectra generated by uracil specific cleavage of the reverse transcript of the IGF2/H19 region. Cleavage products derived from the methylated template contain rGTP at every position where the Cytosine of the forward strand was methylated. In contrast, the bisulfite conversion of non-methylated Cytosine to Uracile results in incorporation of rATP on the reverse strand. This 16Da difference between rGTP and rATP, or a multitude thereof when several CpGs are embedded in one cleavage product, can be detected unambiguously. The calculation of the area under the curve of mass signals specific for methylated and non-methylated template can be used to determine the ratio between methylated and non-methylated DNA used for amplification.

The cleavage product derived from the non-methylated template (CGCAACCACT) (SEQ ID NO: 366) was detected at 3132 Da while its equivalent derived from the methylated template (CACAACCACT) (SEQ ID NO: 368) can be found at 3148 Da.

Reactions where one signal represents the methylated template and a second signal represents the non-methylated template can be used to determine the ratio of methylated vs. non-methylated template by comparing their signal intensities. The nucleotide composition of the measured fragments differs only by a single nucleotide, which ensures equivalent desorption and ionization behavior during MALDI-TOF measurement. Depending on the reference sequence of the target region, multiple signal pairs are available for determining the ratio between signal intensities. This information can be used to assess the degree of methylation for each CpG site independently or, if all CpG sites are methylated approximately to the same degree, to average the methylation content over the complete target region.

A direct correlation can be seen between signal intensity ratios and the ratio of the deployed DNAs. The span of linearity of this correlation ranged from 10%-90% of methylated template. The average standard deviation of the investigated concentrations was approximately 3%, with higher standard deviations towards both ends of the scale. If the ratio between methylated and non-methylated template is below 10% or exceeds 90%, the signals that are representing the lower amount of template can still be detected, but the intensity of signal does not correlate exactly to the actual ratio anymore.

### Example 7

### Methylation Pattern Analysis of IGF2/H19

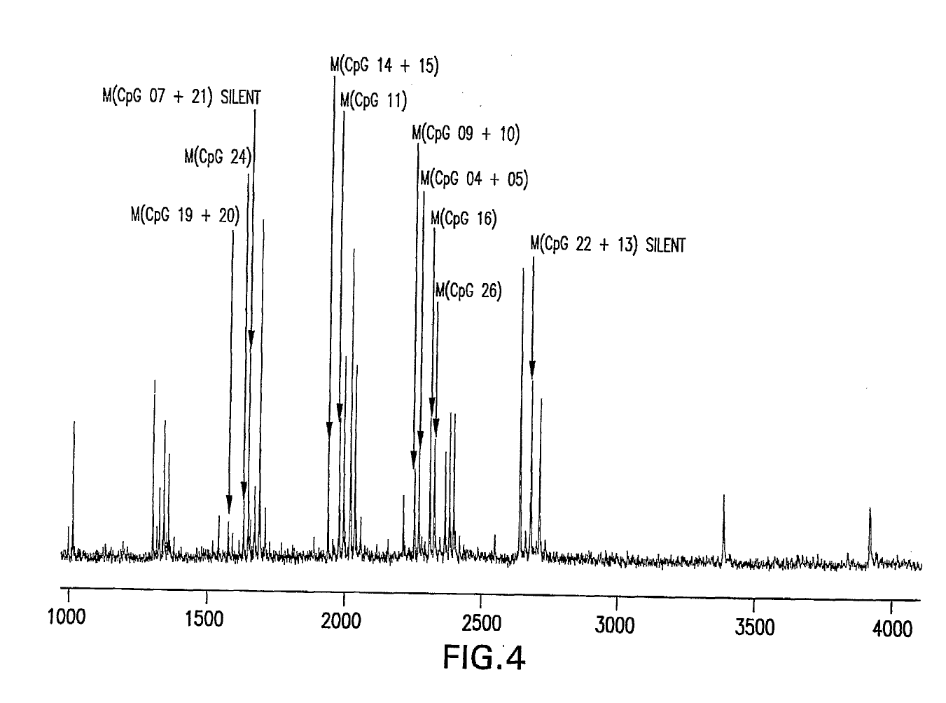

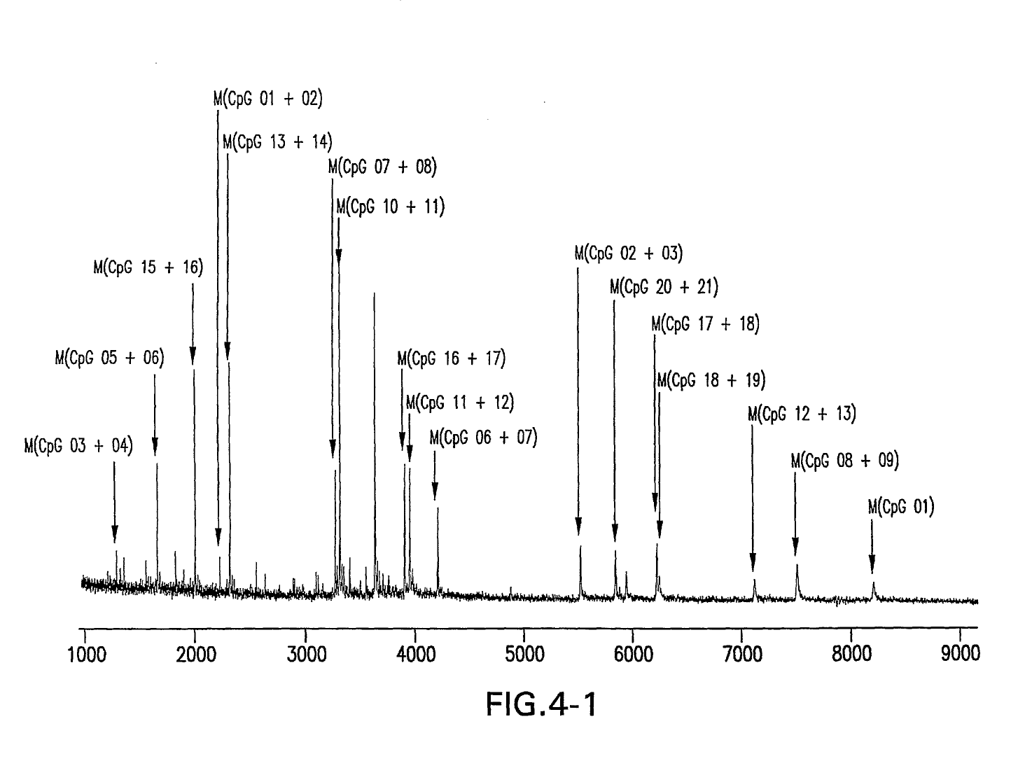

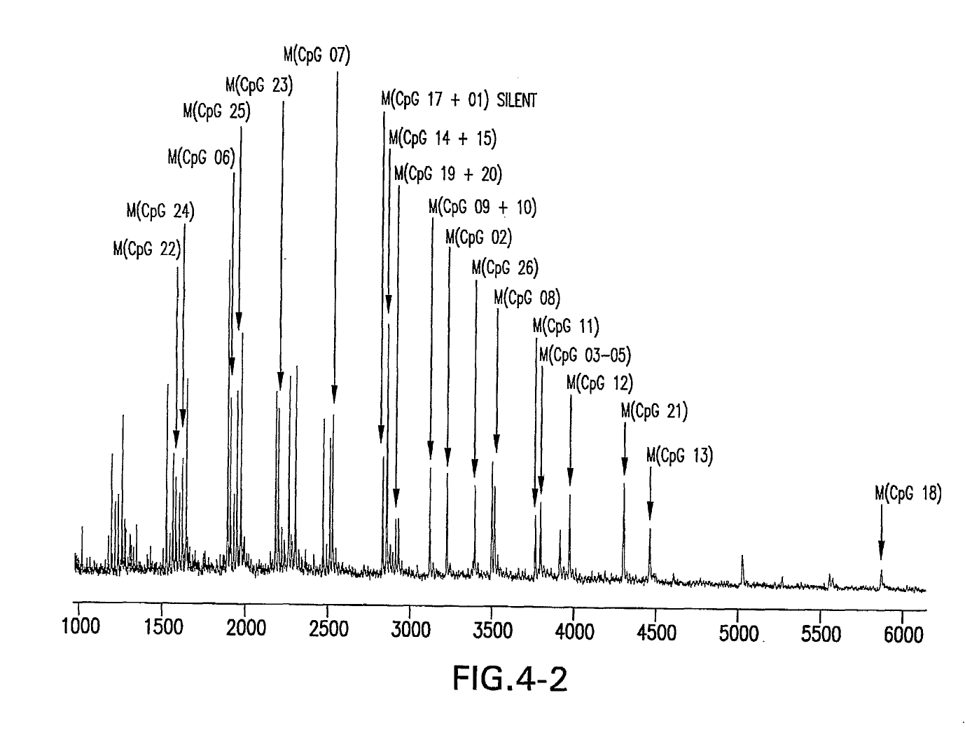

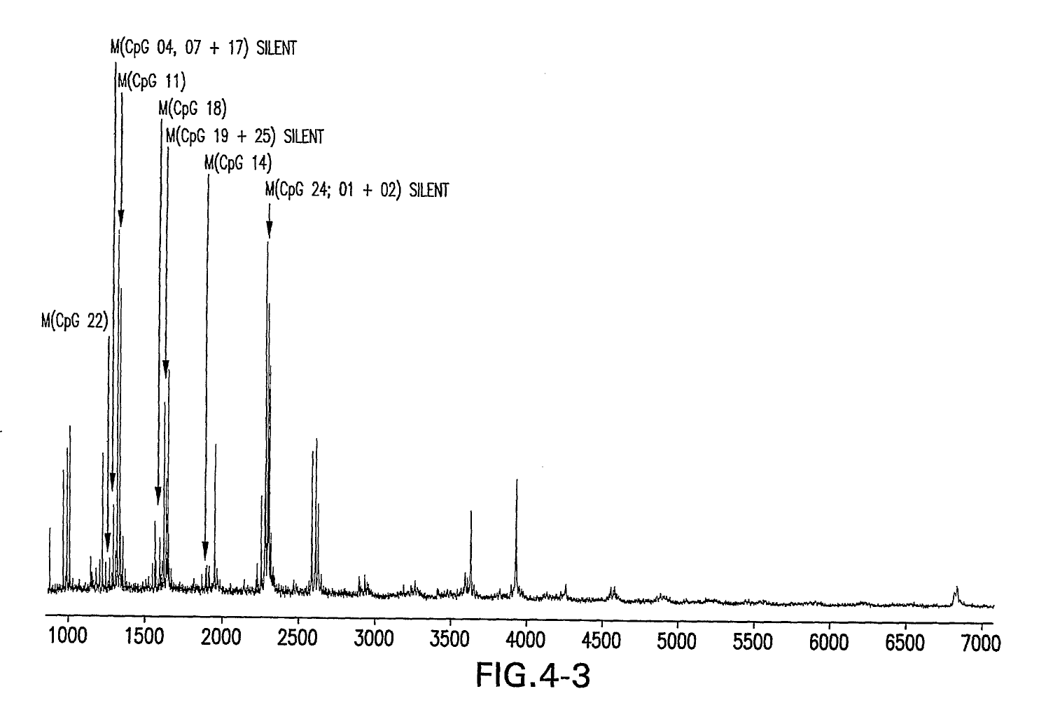

The capability of base specific cleavage to determine the methylation status of each and every CpG within a given target region was determined. As outlined above, the C-specific forward reaction incorporates a cleavage nucleotide for each methylated CpG within the amplicon. The resulting cleavage products represent the existence of two cleavage nucleotides (exception: first and last fragment) or in this case two methylated Cs. Given the current limitations of MALDI-TOF instrumentation, a practical mass window ranges from around 1000 Da to 10000 Da. In this mass window, cleavage products with a length around 4 to 30 nucleotides can be detected. When the distance between two methylated cytosines becomes smaller or larger than this range, the resulting mass of the cleavage product might be too high or too low to be detected under standard conditions. The analysis of a single reaction still allows determining the methylation status of approximately 75% (depending on the reference sequence) of all CpG sites within the amplified nucleic acid molecule. To obtain information about all CpG sites, a set of four reactions were performed: C- and T-specific cleavage of the forward and reverse transcription product. This combination enables base-specific cleavage after each nucleotide (C-specific cleavage on the reverse strand equals G-specific cleavage on the forward strand; T-specific cleavage on the reverse strand equals A-specific cleavage on the forward strand). The combined information from these four cleavage reactions allows compilation of the exact methylation pattern. For the IGF2/H19 region described here, two reactions were sufficient to obtain the methylation status for each CpG site. However using four reactions provides the advantage of information redundancy. In this system 92% of all CpG sites were represented by more than one signal. This means that each methylation event is independently confirmed by more than one observation in one or more reactions. This redundancy is an important aspect in potential diagnostic use. FIG. 4 is a mass spectra representing all four base-specific cleavage reactions of the IGF/H19 amplicon. Numbers correspond to the CpG positions within this target region. Arrows point at the mass signals that indicate the presence of a methylated cytosine at the marked position. All methylated CpG's in the selected region were identified by one or more mass signals. Approximately 75% were identified by more than two mass signals.

The methylation pattern of the IGF2/H19 imprinted region in adult blood samples confirmed the segregation into methylated and non-methylated template strands reported by Vu et al. (Genomics 64(2): p. 29331-40, 1999). Out of the 24 clones analyzed, 13 (54%) could be identified as methylated and 11 (46%) as non-methylated. No sequence changes were observed. Vu et al. (supra) showed by dideoxy sequencing of bisulfite treated DNA that 25 out of the 26 CpG sites within the amplicon are methylated. The only non-methylated CpG was found at position 470. The results indicated a slightly different methylation pattern in the studied sample DNA, where all CpG sites were methylated. The data also confirmed methylation of the CpNpG site at position 347. Due to the variability in individual methylation patterns, which have been observed by other groups, minor differences are anticipated.

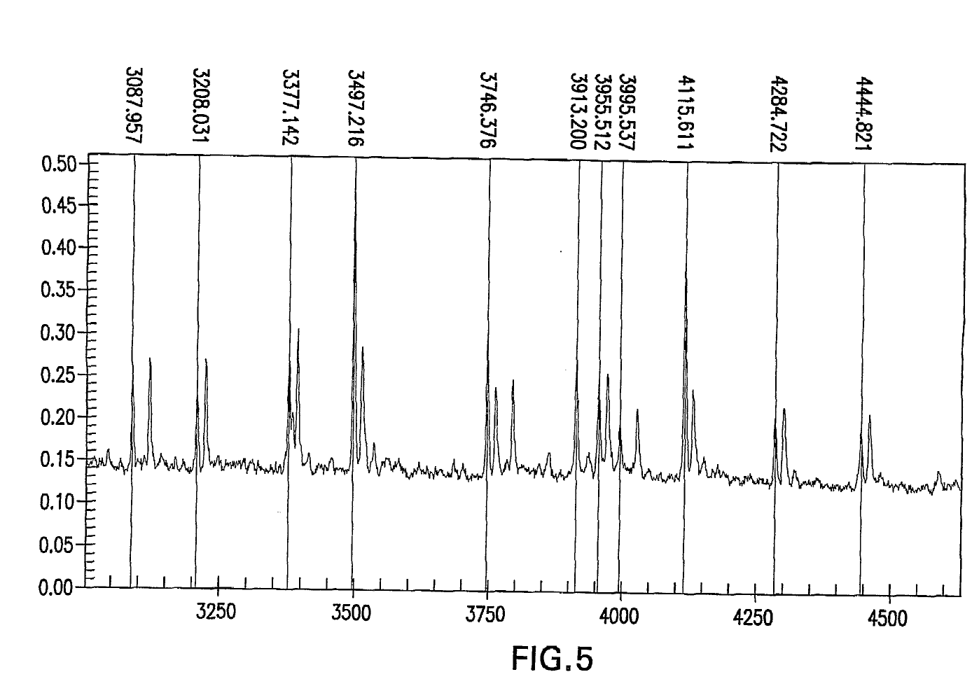

The results demonstrate the capability of the method to discriminate methylated and non-methylated DNA nucleic acid target gene regions simultaneously and to reconstruct the exact methylation pattern. In order to support this contention, bisulfite treated genomic DNA was analyzed directly. The produced mass signal spectra showed signal patterns that are representative for the methylated template as well as those that are characteristic for the non-methylated template. The signal intensities for methylation-specific signals and non-methylation-specific signals were compared and the 50/50 ratio expected for hemi-methylated DNA, as in control blood samples, was confirmed. FIG. 5 is a mass Spectra generated by uracil specific cleavage of the reverse transcript of the IGF2/H19 region. Genomic DNA was used for amplification. Dotted lines mark the position of mass signals representing non-methylated CpG's. Signals with 16 Da shift (or a multitude thereof) represent methylation events. The area-under-the-curve ratio of methylated versus non-methylated template approximates to 1, as one expects for hemi-methylated nucleic acid target gene regions. This indicates an unbiased amplification of methylated and non-methylated template for the analyzed region and validates our semi-quantitative capabilities.

### Example 8

### Analysis of Methylation in AML Patients

In order to investigate methylation in AML, 180 genomic regions were analyzed in 192 samples from adults with AML using Sequenom's proprietary methylation analysis methods and systems. The clinical data for the 192 samples are provided in Table 11. These results were further validated as part of a second phase in an additional set of 72 samples. The approach identified a highly significant methylation-based predictor for patient survival (P<0.01).

Samples

A total of 192 DNA samples derived from peripheral blood (PB) and bone marrow (BM) specimens from adult AML patients were provided by the AML Study Group Ulm (AMLSG ULM, Germany) with patient informed consent and institutional review board approval from all participating centers. Following sample collection, patients were entered into one of two treatment protocols (AML HD98A and AML HD98B, enrolled between February 1998 and November 2001), and received intensive induction and consolidation therapy. The median clinical follow-up was 513 days overall (1120 days for survivors); Conventional cytogenetic banding, FISH analysis, and MLL and FLT3 mutational analysis were performed as previously described (Frohling et al. Blood 100:4372-80 (2002)), Dohner et al. J Clin Oncol 20:3254-61 (2002)) at the central reference laboratory for cytogenetic and molecular diagnostics of the AMLSG ULM. Detailed clinical, cytogenetic and molecular cytogenetic information are provided in Table 11.

Methods

In an initial phase, the methylation status of 180 genes in 192 samples of adults with AML were analyzed to evaluate if such analysis of genomic DNA-methylation provides new insights into the molecular classification of AML. The 180 genes from this first phase included over 6600 CpG sites for each of the samples. The genomic sequences containing the CpG sites are provided in Table 8. The CpG sites were analyzed as 3732 CpG units (where a unit comprises 1 or more sites). All experiments were performed in a first-pass approach.

Amplification of bisulfite treated DNA was performed as described in Examples 1 and 2 using the primers provided in Table 4. Some of the regions have more than 1 set of primers because more than one amplicon in that region was amplified. Sometimes the amplification product is less robust compared to genomic DNA due to the high degree of degradation of the DNA; therefore, a quality filter was applied that served to remove low quality data from the analysis. The analysis of CpG units was restricted to those units that had data available for more than 75% of the samples. After filtering, data for 117 genes (see preferred set) of the original set remained available for further analysis. Also, 10 patients samples were removed from further analysis because of poor DNA quality.

TABLE 4

| | | |
|---|---|---|
| Region | Forward Primer (SEQ ID NOS 1-179) | Reverse Primer (SEQ ID NO: 180-358) |

| | | |
|---|---|---|
| ABO1 | TTGGTTGTTTGGTAGGGGTAGTTAT | ACTAACCACTTTTTCTTTTATAACTTTCAT |
| ABCB1 | TGAAATGTTTTTAATGATTTAGTTGATG | ATCCCATAATAACTCCCAACTTTAC |
| ACTG1 | GGGGTGTTGTAGAATTTTTTTTAGTTTAA | AAAATCCTTATCCCCCATAAACAAC |
| ACTG1.01 | GGGGTTAGGGTTTATTTTTGGGTA | TCTAAACTACTCCCTCCCCAAATCC |
| ACTG1.01 | TTGTTAATGGTGATGATTTGGTTAT | TCCTCCCTAAAACCTCCAAATTTCT |
| ACTG1.02 | GGAAGTTGGGATTTGAGTTGGTTT | CTCCCCAAACAACCCTACCTCTAT |
| ACTG1.02 | TTTTTTTTGGTTTTGTTTTGGTTTG | CTCAACCTCCATTTTCTCCTCTAAAC |
| ACTG1.03 | GGGAGTGGTTGAAATTTAAGTTGAG | TTCCAACACCCAAATCTACTTCCTC |
| ACTG1.06 | GGTTTTGTTGTTGTAGATTTGTTTTATTTA | TCCTTAAAAACCAAAAACTCCTCCC |
| ACTG1.09 | TTTTTGTGGGTTTTAGAGAAAGTTT | AAAACAAACAACTCCCAACACTAC |
| ADFP | GGGGAGTTTTTTATTTTAATTGGG | CTCCAAACAAAACTACCTCCAACTC |
| AFP | TTTATTTTTAGGGAAAGAGGGAGGG | AAAACTACCCCAAACACACTTCCC |
| AGT | AGGGAGGTGGGTAGTTTTGTAGGAG | ACAAAACAAAAACACCCTCATAACC |
| AMIGO2 | GGGTTTTTTTTATTGTAGGTTGAAGGTAT | TCTAATATAAACCCCTACCCCCTCC |
| ANGPT1 | GTTGGGGAGGATTTAGAGGGAGAT | ATAAACAACCCACACCAAAACAACC |
| APOB | TTTGGATTTTGTGGTTGTTTTTTTT | CCCTTTAAACCTTTTACAATCCTAAC |
| APOC1 | AAGTTGGAGGAGTAGGTTTAGTAGATA | AAACTAAAATCCACCCCAAAAAAAC |
| AQP1 | CATCCAGAGGAGGTCTGTGTGGTGTG | GGCTGTCACACTGGGGCTGCTGCTCA |
| ARHGAP22 | GGTGTTTAGAGAAATTTTAGAAAGTTGGAT | CTTCCAACCTCAACAAAAAATAACC |
| ATP8B4 | TTTTTTAGGATATAGGTTATTTTTTGAAGG | CATAACACAACCCAACTTCACCAAC |
| AZGP1 | TTTTTTTGATTTATTTTGAGGTTTT | AAACCCCAAACAACTACACACCTAAC |
| BAALC | GGGAGATAGAATTTATTTGGTTTATTTATA | AATCCTACCTCTACTTCCTCCCAAC |
| BAI2 | TTAGGAGTGTTTGGGTATGGTTAGTA | CCCCTCCCCTCAACTTAAAATTAAA |
| BCL11A | GATTGGGTTTGAATGTAATTGAAAG | AAAAATATATCCCTCCCAAAAACCC |
| C10orf38 | GTTAGGGGTTTTTTTTGTTTTTTTT | AATACTTTATCTCTACAACAAAACTACCC |
| CD3D | GGATTGGTGGGAAAATAAGAGAGTAGATT | TACCAAAACCTAAAATACCAACAAC |
| CDC42EP4 | GATTTTTTTTGTTTTATAGGGGGATT | CTAAAAACTCCCAACCCTAAAAACC |
| CDH5 | CCCTGAGGCAGAGGGTGAGGAGTAG | GCCTGCCTGGGCCTGCTGGCAGTG |
| CDKN2A | TGTTTTTTAAATTTTTTGGAGGGAT | AAAAAAAACCATACTTTCCCTATAACACCA |
| CDKN2A | GGTTGAATGTTAGTTTTGAATTAAAAGT | AAATAAAAATAAACTAAACACAAAAAACTC |
| CDX2 | TAATGGTAGGGTTGGGAAGGTGTATATTA | AATCCTAACTCCCAAAAACCCACTT |
| CEACAM6 | GGGACTCTCTGTGTGGTGCTGACAG | GTGACCCTGGGAAATGCTTCTATCCCTG |
| CEBPA | GGGTTGGAAAATTTTTTTTATAATTATTTT | CACTCAAAAAACCCCAAAACCTAAC |
| CKMT1 | TTGGGGGAGTTTTATTTTTGGAGAT | CTACAAACTACACAACCCTCCAACTC |
| CNN3 | AAGGGTTTTTGTTGAAGTGGGTTAT | ACCTATAACTAAAAAACCCCCAAAC |
| COL1A1 | CATGTAGACTCTTTGTGGCTGGGGAG | AGGAGGAGGGAAGGGAGTCCACCCC |
| CTNNAL1 | TGTGTATTTGGATTAATTGTTATATAGTTT | ACCTTTACCCCCAATACCTACCTC |
| D2S448 | GGGTTTTTATATATTTTTTAGGGGAATTGA | CCAAAAACTAACCCCACTACATCAAC |
| DAPK1 | GTTAGGAATGTGGTTTTGGGGATT | TCAATCTCCAATCCTTTTAAAAAAAA |
| DLK1 | TTTTTTTTGGGGGTTTTTTTGTGT | ACCAATCCCTATAACCCCCTCC |
| DMPK | GGGAAGGGGATATATGAGGGATTTAT | CCAAAAACCACAAACAACCTTAAAC |
| DPEP2 | GGGGTGGTAGTTAGAGAGTTTGAGAG | AAACAACAAAAAAACCACCTAAATC |
| DUSP4 | GGGTTGGAATTTAGTTTTAGTTTTGTTGT | TTACTCCTCCAAATAAACCCAATCC |
| E. cad (CDH1) | GGGTATTGGAGAATAAAGATATTTTTAATA | TCAAAACCAAAATAACAAAACTCC |
| EDG1 | GGGGGTTTTTAGTTGATAGAGGG | TAACCCAAAAATACAAATTTTCAAC |
| EML4 | TTGTTGTTTGGGAGGGAGGT | AAAAAAATTCCCACTTTAAAAAAAC |
| EMR1 | TGTTGTGATTTGGGAGAGGTTTAAG | CCCACCTACTAAATAAAACCCAAC |
| ERalpha | TTTTTATATTAAAGTATTTGGGATGGTTTT | TCCAAATAATAAAACACCTACTAACC |
| ESR1 | GGGAGATTAGTATTTAAAGTTGGAGGTT | AATCTAATACAATAAAACCATCCCAAATAC |
| ETS1 | GGTATTTTAGGGGAAGTTGGTATTTTG | ACCTATACACCCAACCTACACACCC |
| EVI1 | AGTGTTAGGAATTTAGATTTTGGTAAT | AAAAAACTCCTCACTTTAAAAAAAA |
| FARP1 | GTTTAGAGAGAGGGATTGGAGGTTTAGA | AAAAAACAATCTTCAAAAACCCACC |
| FGFR1 | GTTTTTTGTAGTTGTTTGTTGGGTTTTG | AAACACTATTATCCCCCATTTACAAATAAA |
| FHL2 | TTTTTTGTTTGTTAGGGTTTTTTTT | AATAAAACCTTCCTTTAATCCCCTCC |
| FLI1 | TGTATTTTTTAATGGTTGGTTTGTTT | CCCTCTTCCTCCCCTACTAATCCTAC |
| FLJ21820 | TTGGTTTAGGGTAATAGGGGTTTTG | CCAAACCAATAAAAAATCTCCCAAC |
| FLJ23058 | TGATTTTTATAGAGTATGGGTGGG | AAAACCCATAAAAACCACAACCC |
| FLJ25409 | ATTAGAGTATGATTTAGGTTTTTGATAGTT | AAAACTAACATTTTCAACAAAAACTC |
| FLT3 | GTTGTAGGTGGTTTTTTTAAGGATG | AAAACCCTACCTATTTTTCTTAATCCC |
| FN14 | TAGGATTTTGTTGAATGAATGATTGAATT | TTTAAAAACCACCTAACCCCAAATC |
| FOXO1A | GGGGAGGAGATTATTTGGTTTTTTT | CCCCAAAACTTTAATCCTATCTCCC |
| GAGED2 | GGGACCTGGGAAGGAGCATAGGACAG | GGCCATAACTAGGGAGGAAGGAGGGC |
| GAS7 | GGGTTTAGGGGGAGGAGATTTAG | AAACAAATTCAACCCCAAATTCAAC |
| GLUL | GAGGAGAGTTTTTTGGGGAAATG | ACTCTTCCAAACCTTAAAAACCCC |
| GNG2 | GTAGGTAGTGTGTTAGGAAGGGGGT | CCAACCCAACCCAACAATAATAAAA |
| GS3955 | GATTGTTTTGGGGTAATAAAAAGATT | TAATCTCCCTCCAAAAATTCCAACA |
| GSTP1 | TGGGAAAGAGGGAAAGGTTTTTT | CCCATACTAAAAACTCTAAACCCCATC |
| GUCY1A3 | GTAGTTGGGGGATGTTTGGATTT | ACCCCTCACACCATTATCACTATCAA |
| GYPC | AGGGTTTTGGGGATTTATTGGAG | AAAACAATTCTAACCCCACACATTTC |
| HIP1 | TTTAGGTTAGTTGGGGTATTTTGGG | CTAACAAAACTCCAAACCAATCACC |
| HOXA1 | TTTTTTTTTAGTGTTTAGTTTAGAGTTTG | AAAAAACAAACATCTTCTCTTTCCCTACTA |
| HOXA10 | TGGTTGATATTTTTTGTGTAAAATATGTTG | TCAAACAAAAAACCAATTCCAAATC |
| HOXA10 | GGGTATTATTGGTTTAATGGGGAAG | AAAAACCCAAAACCCTAATCCCTAC |
| HOXA11 | TTTTTTTTGTAGTTATTTTAGGGGAAGTAA | AACAAACCACCAAACAAACACATC |
| HOXA3 | TTTTAGGTTTGGAGGTTGGTTAGGT | AACCACTTTTTCTTTTATAACTTTCATATC |
| HOXA4 | TGGATTTTTTTTATTTAGGGGTATA | CTACAACAACCCCAACTCCCTC |
| HOXA7 | TTAGAATGGAAGGGTAAGAGGTTTAAAT | AATCCAAAACTCACTAACAAAAATC |
| HOXA9 | TTTTTTTTATTAATTGGAGGAGAATTATAA | CCACTAAAACCCTAAACAACTACTAC |
| HOXA9 | TTTAGGGTTTTAGTGGTGGTTATTAT | ACAAAAACAAAACTAAATTTAATCTTTTAA |
| HOXB2 | GAGAGAATTTTGTAGGTTAGGGGAGAG | CAAATCAAAATCTAATTTCAAAACC |
| HOXB2 | GAGAGAATTTTGTAGGTTAGGGGAGAG | CAAATCAAAATCTAATTTCAAAACC |
| HOXB5 | GAAGGTTGGTTTTGGTTTTTGAGTAGA | CCCCACCCACAAAAAAATAAATAAAA |
| HOXD11 | TTAGTTTTTAGGGAGTTTGGAGT | ATTACACAAAAAACTTAAACCAAAATCAAC |
| HOXD13 | GAGTGGGTGGGTTTAGTTAGGTTTG | ACCCTCTCTCCCTCTATAAACCTCC |
| ID3 | TATTAGGGGGTTTAGGGGTTGGTT | TAAACTCACTCCCCAACATAAAAAC |
| IFI27 | GGTAGAGTAGAAGGGTTTTTGTTTTTT | AAACAACCAATCAAATAACTAAATTTACCA |
| IL6ST | TTTTTAGGGGGAAGGGAGGTTT | AAAAATCTCTCAAAAACCAATCAAC |
| ISG20 | TTTAGGTAGAGGAGTGGATTGGAGT | ATCCTAAATCTCACCTAAAACCCC |
| KIAA0476 | GAGGTTATTAGGTGGGATTTTTTGAG | ACCCCCAACTACTATCCCTCACTAC |
| KIAA0830 | TTGGTTGGGTTGTTGGAAGGT | AAAAAAAACCTCCTCCCACAAAAAA |
| KIAA1447 | GGGGTGTTGTAGAATTTTTTTTAGTTTAA | AAAATCCTTATCCCCCATAAACAAC |
| KRT13 | TATTTTGTTTAGGTAGGAGGTTAGG | CAAATTCCTCAAAACTCAAATATCC |
| LAD1 | TTTTTAGTTTAGGTGGGATTATATGGT | ATACAACTCAAAAAACAATACCTCATTCAT |
| LAMB3 | TTTTGGATAAGGGAAGTTGTGTATT | AAAAACTTCAACCACCAAAAAAC |
| LCN2 | TTTTAAAGGTTTTTGGGTAGTGATT | ACAACCTAACACCCCACTTTACCAT |
| LGMN | GTTTTTTGTGGGTGTGGTTTTTTA | AACCCAAAAAATCTCTCCAATTACC |
| LOC114990 | TGGTGTTTTATAGGTATTTGGGTTGTG | TCCCCCTCAAAAAAATTTAATTCATAAA |
| LOC55971 | TGGAAAGTTTTGATTTTTTTGAGTTT | CCCATTCCAACTACCTAACCCC |
| LOC57228 | AGTTTGTTAAGTTTTATTGGGTTTTAGTT | AAAAAAACAAACTACCTTTCCTCCC |
| LRP6 | GGAGTATATAGAAGTTGTAGGTTAGGAGGT | CAAAACCTCTCCCAAAATCTCAAAC |
| MAGEA3 | GGGTCCTGACCTTGATTCCTGCCACAG | GCAGGGGTGGAACTGGATTCTGC |
| MAP7 | TGGGTTTTTGTATAGATTAAAAATAAAAA | AAACCAAACCAATAACCAAAAAATC |
| MEIS1 | GGTAGTTTTTGTTTGAAATTTTAGTTTT | AATCTAAACTCCCCCACCTCCTAAC |
| MGC14376 | ATATTTATTTGGTGTTGGGTGTGGG | AAAAATAACCTCCTTACCAATCAAAACC |
| MGC16121 | GATGGTTTAAGATTTATTTGTTGGGTAGGT | AACTTCCTTCAATCATCCAATCTTTATTC |
| MGMT | GTTGGTTTGGGGGTTTTTGATTAG | CCTTTTCCTATCACAAAAATAATCC |
| MGP | GGGCCTGTCTTCAGAAGAGAAAATGG | GGAAGGCTGAACTGCTGAGTCTGAC |
| MIG2 | TTGTTTTTTTATGGAAATGAAGGATT | CCCCAATCCAACCTAAACTCTAAAC |
| MSLN | GAGATGTTGGTTTTTGTGGGAAGTT | AAATCACACAAACCTCCTCATTAACTACT |
| MYOD | GGGGATAGAGGAGTATTGAAAGTTAGTTTA | AAAACTTCCTCACCCCTAACTTCTC |
| N33 | CCTCAGATTGAGGTCCCAGGGCCAAAGGA | GAGGTAGAATGGATCCCCTTGGCCTTC |
| NBL1 | GTAGAATTGGGGATTTTTGGTGT | CAAAATAACTCCCTCCAAACAAAAC |
| NFIB | TTTAAATAAAGTAAAGGAATGGGTTTT | AATATATTCTCCCATCTATCTCACTCAAA |
| NFKB1 | TTAGTGGGAATTTTTAGTTAGGAAGTGAG | CCTCTCAACTACTATCAACCTCCTCC |
| NFKBIB | TCAGTGGGAATTTCCAGCCAGGAAGTGAG | GGAGGAGGTTGACAGTAGCTGAGAGG |
| Notch4 | TTTAGGGTTATTTAATTATAGGGTTAGTTA | AAAAAAACTAAACCACCAAAAACCC |
| NR2F2 | TGGGGATTGAGGTTGGTTATTAATT | CTCCCAAATTCTCTAAACCCCAACT |
| NRP1 | GGAGATTGGGAGGAATAATTTTTTTT | CTCCCTAACACCTAAACTCCCAAAC |
| p16 | TGTTTTTTAAATTTTTTGGAGGGAT | AAAAACCCAATCCTCCTTCCTTAC |
| p53 | GAGTTTTAGGGTTTGATGGGAA | CCAATTCTTTTAAAAACACTATATTCCTTA |
| PAGE5 | TGGAGTGGGTAAGATCATTGCAAGCATGAC | TAGGTCTGCAGAGTGGTCTTCCTGGTA |
| PBX3 | GAGGGTATTATTTTTTGATAGGAAGAG | CAAACTACCAATACCACTCACTCACTAC |
| PHEMX | ACAAAGCTGGGTTCCTGCTGGGCCC | GGAGCAGCACCCTTCCAGGGGAGGTGG |
| PIK3R4 | TTAGTTTTTTAATTTGTTTTGGGGGATAT | TTTATCAAAACATCATTTTCTCCCTATAA |
| PITX2 | GGTGTGTATTTTTAGTTTGTGTTTGGAG | AATACCCTTCTACCCACATCCCATAT |
| PLCG1 | GGAAGATTTTTTAGGTTAAGTTGGAGA | AAAAACCACTACCTTAACTCCCCTC |
| PLEKHC1 | TTGTTTTTTTATGGAAATGAAGGATT | AACAAACCCCCTCTCCCTACTACC |
| PMP22 | AGTTTAGGTTGATTTAGAATAGGATTTTG | CCCCTAAAACAAAAATAATAACCAAC |
| PRAME | CCTGCCCTTGGCTGGGTAATCTCTG | GTCTGGGGCCAGCAGGGGGCACTA |
| PRG2 | ATGGATTTTAGGAATTTGTTTAAGGTTAT | ATAAAAAATCTACCCCAACCCCTTC |
| PRO2730 | TTTATTTGTTTTTTTGGTAGTTATAGAGTA | CACAATCCAAACAAAAAACCCTC |
| PSCB5 | TTGTTGATGTTATATTTTTAGGTTTTAATT | CTAAAAAACCCCACACCCCAAC |
| PVALB | GTTTGGGATTGTTTTGGAGGTATAG | AAAAAAACAAACAAATAACCTACCTCTCAC |
| RARB | TTGTTTGTTTTTGTAGGGTTGTTGG | AATTCCCAAAAAAATCCCAAATTCT |
| RASSF1 | GTTGTTTTTTGGTTGTTTTTTT | ATCCCTACACCCAAATTTCCATTAC |
| RBP1 | TTTAATTTGTAGTTTGGGGGTTGTTTT | TAACCAACTAACTCCAATCACTCCC |
| RGS16 | ATTTTTTTAGGTAGGTGGTGGGGAA | CCTCAAAACCCAACAAACTCAAACT |
| RIS1 | GAGGGGAAAGGGTTTTATTTTTTTT | AACCTACTTCATAACCCTAATCATC |
| RPL22 | TTTTTTTTAGGTTTGGAGGGTTTTTG | AACTACAAAAAATTTTCCCACTTCCC |
| RUNX3 | GGGAGAGTTGGTTTTTATTTATTT | ACCCACAAAAATCCCTCATTCTCTA |
| S100P | GTGAGGGTTTTGATTTTAGAATTAA | CTACCTCCACCTCACTCTTAATAAC |
| SATalpha | AAAGAAGTTTTGAGAATGTTTTTTTT | TTCCAAACACACTTTATATAAAATCTACAA |
| SCAP2 | GGTTTTTAATTTTTTTAGGGAGGGG | CCCACCAATAACTCCTCCTCCTACT |
| SDK2 | CTGGTGACAGCCAGGTAGGTGGAAGTTT | CCACTTTGGGTCTAGGGAGAGGAGG |
| SDS-RS1 | TATATGGAGGTTTTGTTTTGTTTTAAAAA | AAATAAACCAACCCTTACCCAATCTC |
| SELENBP1 | AGGGAAGAAGTGACCCTGGCTGATG | GGGTGGATCACCTGAGGTCAGGAGT |
| SEMA3F | GTGGGAGTTGTTGGTTTGAAATAAG | CCCTAACTTTATCTCTCTATAAATACACC |
| SERPINA3 | TTTTTTTGGGTAGTAAAGTGTTGGG | AAAACTCAAAAAACTTATCTTTAAAACACA |
| SERPINA5 | TTTTTTTGGGTAGTAAAGTGTTGGG | AAAACTCAAAAAACTTATCTTTAAAACACA |
| SERPINB5 | GCTGTGGGGTGGGGCACACTTG | TTACATGGAGGACCTGCAGGAGCTCACCAT |
| SFTPB | TGGGGGTTTAGAGGTATAGTTTTTT | CCACACCTATCTAAACACCAAAATC |
| SLC2A1 | GGGTTTGGGGTTTAGTTTGTTTTG | TAAAAACTCCAATCCAACTTTCCAC |
| SLC6A8 | TTTTTTGTTAGGTAGGTTTTAGTTATTGT | CCAAACTACCAAATCCCCCTACTC |
| SLC7A5 | TGATGTAAGGATGTAGGGATTTAGAGATTA | TCAAACCAACCCTAATACACTACCC |
| SLC7A7 | TCTGGCTGTGGGGGACCAGGAC | AAAGTGGGCTCCACTAAGCTGGGAAGG |
| SMG1 | TGAGTAATAGGGAGGGTTTTGGATTT | AACTAAAATAACTAACAACCCAAATAAATA |
| SNX9 | GGGGATTTTTAGGAATTGTAGGAG | CCATACCCAAAAAAAACTAACTAAACC |
| SOCS1 | AGATTTTTTTAGGAGGTTATAGAAGGTGTT | TCCAAATCCAAAACTCCCAATCTAC |
| SPI1 | AGTAAGTTAGGAGGGTAGTGGGTGG | TATCACCCCAAAAAAACTATCTCCC |
| SPUVE | TTTTTTGGGTTGTTTTATTTTGTTT | AACAACAAAATCTTCTTTCCCCATC |
| STGB3A1 | CTGGGGCCCTCTGAGAGCAGGCAGGC | CCAGTGGATGGGCCTGGTTTGTTCC |
| STX1A | GTAGAGGGGGAGTTATAGGTGATGG | CCCCCAACCAAAACTAAAAAAAAC |
| TACSTD2 | GGAGGGGAGTTTATTTATTTTTTTAATTTT | ACCTCTTAATCCCCTCCCTATTATACC |
| TBXAS1 | TTTTTAGAATTTTGTGTGTGTGTGTGTA | AACAAAACATCCTATCCAAACATCC |
| TCF4 | AAATTTTGTTGTATTGAGATATTTTAATGT | AAAACTAATACCAAACAAAAACCCC |
| TGM2 | GGATGGGGAAACTGAGGCTCCAAGCA | GACCTGGGAGGCCACCCATTGCCCA |
| TM4SF2 | TGTATAAAGTAGAAATTTAAATGTTAGGG | CACAAATTTAATCTCCATTCTCCTC |
| TMEPAI | TTTAGGGAAATAAAATGGAAATTTTA | CATAAAAATCAATAAATAACCCCAC |
| TNA | GGGTGTTCCCTGGCAGAGAGGCTCT | GCCCAAGAAGATTGTAAATGCCAAGAAAGG |
| TNFRSF12A | TAGGATTTTGTTGAATGAATGATTGAATT | TTTAAAAACCACCTAACCCCAAATC |
| TRIB2 | GATTGTTTTGGGGTAATAAAAAGATT | TAATCTCCCTCCAAAAATTCCAACA |
| TUBB | GAATTAGGGGGAGGGGTTGTTT | AAACCATCTTCCTCCCCTACAAAA |
| TUBB5 | AGTTTTTTTGTTTTTAGTTTGGTTTTGTTA | AAAAAAAATCCCTACACCACCTCC |
| TUCAN | AGTTTGATTTTTATTTTGGTGTAGTTT | AATACAAAAAACACAACCCCTACAACC |
| UGCG | TGGTGTTGTGGTTGATGTATTTTATG | CAATCTCCTTTAACCTAACTAAACAATC |
| UGCGL2 | GAGGTGGTGATGTTTAGGGTTAGAG | TCCAAATTTTAACAACTCCAAAACC |
| URB | GGGTAAGTTTGGTATGGTGTTGTTG | ACTTAACCTTCCTACTCCCCCTCC |
| VIL2 | AAGTTTTTGAGAAATTTTTTTAAAAATTGT | AAACAAACAACCTCCCCACTTACAT |
| ZD52F10 | GATGGTGTTAGGTTTTTGGTTTGG | CCTAAATTCTCCCTAAACCCCTCCTA |

Next, a two-dimensional hierarchical cluster analysis (see Example 5) was performed to explore associations among patients and to explore the relationship of the relative methylation of CpG units within and between genes. (See FIG. 6A). The resulting patient clusters were not well defined, and hence a strong correlation to clinico-pathological features could not be observed. However, samples with karyotype t(9;11) and inv(16) did cluster together. A single sample with normal karyotype and two samples with a complex karyotype was identified that presented with generally hypermethylated DNA and deviated the most from the methylation patterns of all other samples.

In general, the clustering of relative methylation in CpG units revealed two main groups: a larger group that is characterized by low levels of methylation and little variation across the samples; and a second, smaller group of CpG units that is set apart by high levels of DNA methylation but the variation of methylation levels across samples is limited. In both groups the variation of methylation levels across samples was limited. However, in both groups a small subset of CpG units splits off early, which is characterized by average methylation levels and higher variation of methylation levels.

The formation of sub clusters among the CpG units is mainly determined by their chromosomal location. In general CpG units from the same gene are clustered closely together. The majority of regions showed constant methylation levels throughout the entire amplification region. A subset of regions showed variable methylation ratios along the analyzed sequence. (See FIG. 6B).

The samples used in this study were derived from either bone marrow or peripheral blood prior to treatment. The peripheral blood samples were enriched to a fraction of blast cell of more than 80%. To investigate weather the methylation patterns are influenced by the sample material, the mean methylation value for each CpG Unit was calculated across all samples derived from peripheral blood and for all samples derived from bone marrow. A regression analysis reveals a strong correlation between the methylation ratios in PB samples and BM samples (R squared=0.99). A t-test reveals no statistically significant difference between the two groups (P=0.61) (See FIG. 6C).

The variance of the degree of methylation for each CpG unit was calculated to obtain a measure for the DNA-methylation variability across samples. The distribution of variance values is shown in FIG. 6D. The majority of CpG units have very low variance values (708 or 52%<0.01, and 185 or 13%<0.001).

Aside from the variability in the entire sample set, the differential methylation in the subgroup of patients with normal karyotypes was also investigated. Segregation of normal karyotype AML samples into two groups based on high and low levels of DNMT3a and DNMT3b expression has been described in the literature (Bullinger L. et al. N Engl J Med 350:1605-16 (2004)). Therefore, the Applicants evaluated if elevated expression of DNMT methyltransferase is correlated to higher levels of methylation by calculating CpG unit specific methylation medians for samples with low DNMT expression (Group A) and compared them to methylation averages for sample with high DNMT expression (Group B). Using a paired t-test, a small difference in the mean methylation levels was observed, which was statistically significant (mean difference=−2.7%, P=0.05, 95% confidence interval: −4.8% to −0.02%, paired-t test).

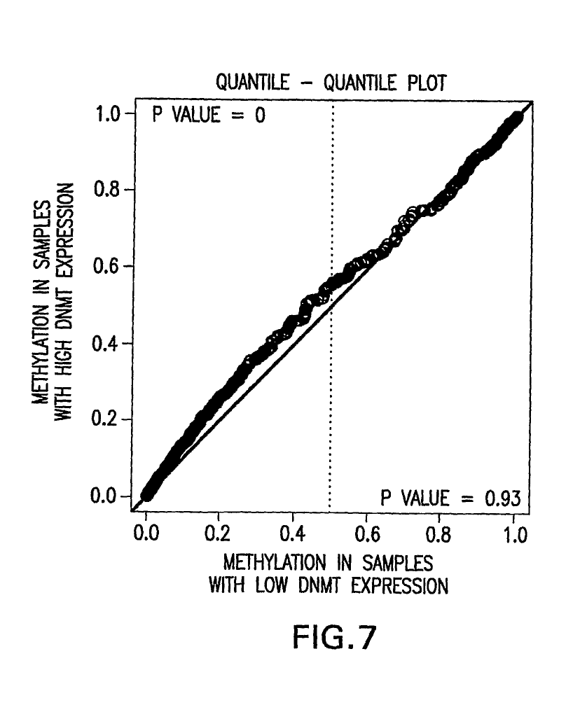

A qunatile-quantile plot revealed that the most prominent differences occurred in CpG Units that are less than 50% methylated in the group of low DNMT expression. To verify this observation, the CpG Units were divided into those with less than 50% mean methylation or more than 50% mean methylation in Group A. The results confirmed a larger difference in CpG units with lower methylation in Group A. The mean difference for those CpG Units was 3.1% (P<0.001). The difference in mean methylation for CpG units that showed more that 50% methylation in Group A was negligible (−0.2%) and no longer statistically significant ((P=0.93), 95% CI=−2%−2%). (See FIG. 7).

Replication in Second Cohort

An objective of this study was the evaluation of a possible correlation between patient's prognosis and quantitative methylation patterns. The two dimensional hierarchical clustering revealed segregation of patient samples into two main clusters. A Kaplan Meier analysis of these two main clusters showed a small difference in patient survival between the two group. (P<0.05,hazard ratio:1.47, CI:1.01 to 2.15)

DNA-methylation patterns at the time of diagnosis clearly correlated with clinical outcome, therefore, a DNA-methylation-based outcome predictor for AML was constructed. However, there exists no consensus yet on how to handle large amounts of quantitative methylation data. To some degree the problem is comparable with microarray-based gene expression studies, where predictive models are built based on thousands of data points. Both, supervised and unsupervised strategies have been used to identify prognostically relevant gene signatures. Recently, a strategy for outcome prediction that combines the strengths of both approaches; a so-called semi-supervised approach has been developed (Bair and Tibshirani, PloS Biol 2:E108 (2004)). Supervised analysis based on outcome is used to select gene candidates followed by unsupervised principle components analysis to build a continuous predictor for survival. This supervised principle components analysis (SuperPC) has been shown to yield reliable predictors for several microarray based gene expression data sets including AML gene expression data (Bair and Tibshirani, PloS Biol 2:E108 (2004)). The SuperPC analysis yields a continuous score for each sample with higher scores predicting worse outcome. Based on this score, samples can be divided into discrete groups characterized by high and low scores (or poor and good outcome), respectively.

To apply this model to the data set described herein, the 192 samples were first separated randomly into a training set (n=89) and an independent test set (n=93). Where applicable, the Chi-Square test statistic was used to exclude significant differences between the sets in clinico-pathological features like: gender, karyotype, cytogenetic risk group, survival time or survival status. (See Table 5). The predictive model was built based on the data from the training set. The resulting good and poor outcome groups showed a significant difference in survival (P<0.001, log rank test: FIG. 8A). This model was applied to the data in the test set and assigned good and poor outcome class labels. Samples assigned to the poor prognosis group were associated with significantly reduced survival compared to samples in the good prognosis group (P=0.028, log rank test: FIG. 8B). The confirmation of the predictive capabilities of the model on the test set data was encouraging and justified further validation. An additional, independent set of 72 samples was collected from patients with AML. Methylation measurements were calculated for all 117 genes which had been previously identified to yield good quality results. Finally, the methylation based prediction model was applied to the validation data set. Again, the model assigned good and poor outcome class labels, which were correlated with patient survival (P<0.001 log rank test). See FIG. 8C.

The superPC algorithm used here also assigns an importance score to each of the features in the model. The CpG units most predictive for survival were derived from two genes located on the long arm of chromosome 17. Notably, the strongest predictor for survival (KIAA1447: accession number AB040880) is a hypothetical protein of unknown function. Methylation of the KIAA1447 gene region is associated with poor survival. Other genes with very high importance scores include one more hypothetical protein (ZD52F10: accession number NM_{—}033317), four genes involved in transcriptional regulation transcription factors (HOXA1: accession numbers NM_{—}153620 and NM_{—}005522; PITX2: accession number BC013998; RUNX3: accession numbers NM_{—}004350 and NM_{—}001031680; NFKbeta1: accession number NM_{—}003998), one actin (ACTG1: accession number NM_{—}001614), one Cadherin (CDH1: accession numbers NM_{—}004360 and AB025106) and one Phosphatase (DUSP4: accession number NM_{—}001394). The list also contains a gene involved in cell adhesion (FARP1: accession number NM_{—}005766)), which was recently found to be hypermethylated in AML cell lines (Gebhard et al. Cancer Res. 2006 Jun. 15; 66(12):6118-28). Gene regions for analysis herein can comprise a sequence from one or more of these regions.

TABLE 5

| | Training | Test | |
|---|---|---|---|
| | set (N = 89) | set (N = 93) | Test Statistic |
| Gender (female) | 45% (44) | 49% (46) | Chi-square = 0.31 |
| | | | d.f. = 1 P = 0.575 |
| Cytogenetic | | | Chi-square = 12.12 |
| Group | | | d.f. = 11 P = 0.355 |
| 8 | 3% (3) | 1% (1) | |
| Complex | 7% (7) | 6% (6) | |
| Karyotype | | | |
| del (9q) | 0% (0) | 1% (1) | |
| inv (16) | 12% (12) | 11% (10) | |
| inv (3)/t (3; 3) | 2% (2) | 2% (2) | |
| normal karyotype | 46% (45) | 44% (41) | |
| other | 5% (5) | 6% (6) | |
| t (11q23) | 1% (1) | 2% (2) | |
| t (15; 17) | 11% (11) | 12% (11) | |
| t (6; 9) | 0% (0) | 1% (1) | |
| t (8; 21) | 8% (8) | 9% (8) | |
| t (9; 11) | 4% (4) | 5% (5) | |
| risk.group | | | |
| 1 | 32% (31) | 31% (29) | |
| 2 | 56% (55) | 59% (55) | |
| 3 | 12% (12) | 11% (10) | |
| Survival Days | 184.25/ | 220.75/ | F = 0.1 d.f. = 1,190 |
| | 531.00/1025.25 | 499.50/1008.00 | P = 0.75 |
| Survival Status | 58% (57) | 59% (55) | Chi-square = 0 |
| (dead) | | | d.f. = 1 P = 0.961 |

Prediction of Disease Relapse

A prognostic set of genes that allows prediction of disease relapse was also evaluated. The method yielded a model with marginal significant difference (p.value=0.05) between the resulting groups in the test set. There is a chance that the observed effect can be attributed to chance alone, because the model didn't show reproducible results when the analysis parameters were altered.

Methylation-Based Prediction Combined with Gene Expression

In multivariate analysis a combined gene expression and methylation-based outcome predictor outperformed the cytogenetic based risk stratification in the data set [odds ratio=4.66 (2.27 to 9.58), P<0.001].

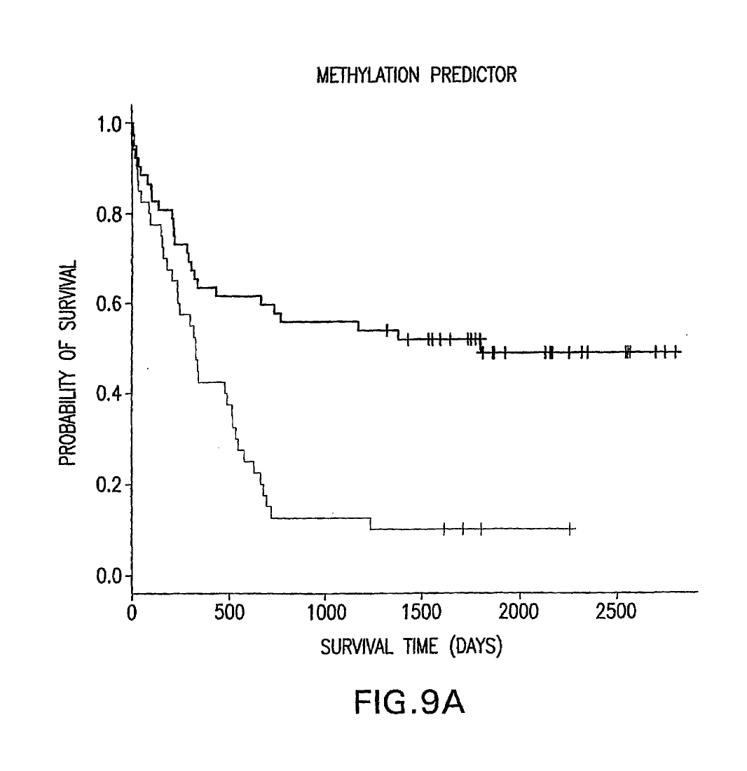

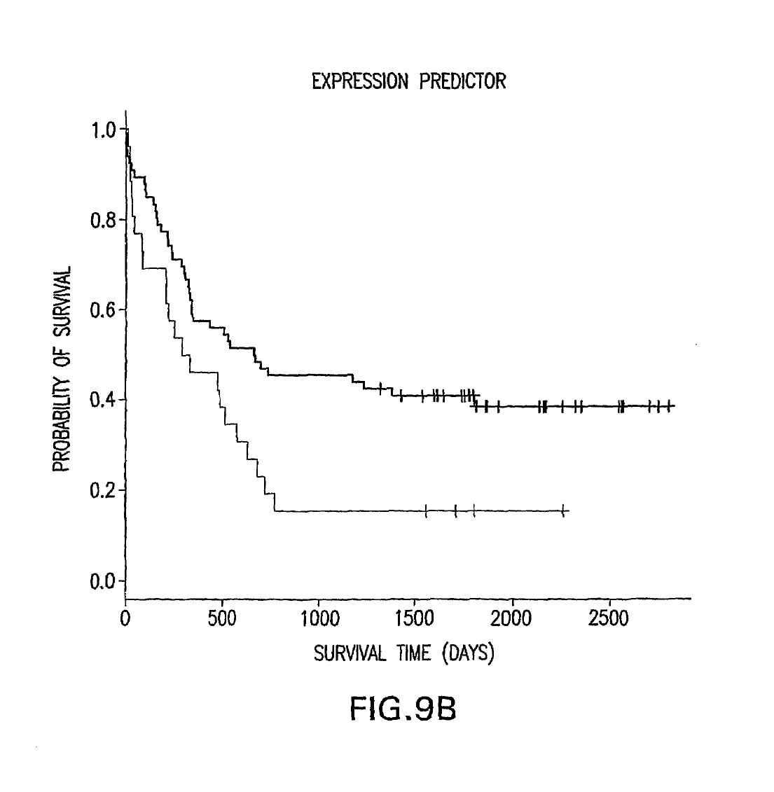

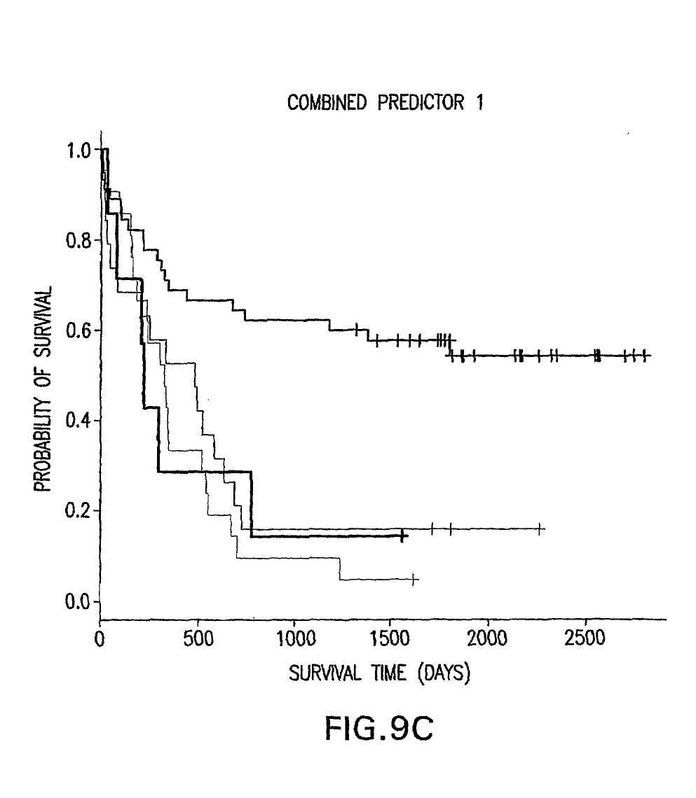

A subset of the samples (n=96) used here had been previously analyzed in a microarray-based gene expression study (Bullinger L. et al. N Engl J Med 350:1605-16 (2004)). Therefore, the inventors were interested to evaluate the concordance of survival-associated outcome labels between the expression- and the methylation-based models. In this sample set, both models were in agreement for 64 cases (70%) and assigned different class labels in 28 cases (30%). In 45 cases, both models predicted good survival (group GG). In 19 cases, both models predicted poor survival (group PP). In 21 cases, the expression-based model predicted good survival and the methylation-based model predicted poor survival (group GP). In 7 cases, the methylation-based model predicted poor survival, and the expression-based model assigned a favorable outcome (group PG). A Kaplan Meier analysis was performed to evaluate the survival times of each of the four groups. (See FIGS. 9A-C).

Interestingly, Kaplan Meier analysis revealed that the subgroup GG (in which both models predicted good survival) was in fact associated with longer survival times. However, when one or both of the models predicted poor outcome, the probability of survival was dramatically reduced. Consequently, samples in the GG group were assigned to a good outcome class, and samples where at least one models predicted poor survival were assigned to a poor outcome class. The association of this combined outcome predictor to survival was very strong (P<0.001, likelihood ratio test).

This model was also applied to the clinically important subset of intermediate risk patients (as determined by cytogenetics). This sample set consisted of 45 samples in the intermediate risk group (37 samples with normal Karyotype). In this subset, 16 samples were assigned to a favorable group and 29 samples were assigned to a poor outcome class. The difference in survival times between both groups was again statistically significant (P<0.05, likelihood ratio test). When the model was applied to the subset of samples with normal karyotype alone, only 9 patients were assigned to the good outcome group. Although visual inspection of the survival curves suggests similar results to previous analysis, a reliable survival analysis is impaired by small sample numbers in one group.

Multivariate Analysis

To evaluate whether the methylation-based outcome predictor adds improved prognostic information beyond known prognostic factors, a multivariate proportional hazard analysis was performed. Using the methylation-defined outcome-class labels (FIG. 9A), the DNA-methylation predictor provided significant prognostic information [odds ratio=3.51 (1.87 to 6.61), P<0.001], independent of other risk factors determined to be significant in the model, which include expression based outcome class, the presence of a FLT3 aberration, and stratification into low, medium and high risk group based on cytogenetics. In this data set, methylation proved to be the most significant predictor for survival. (See Table 6).

TABLE 6
Multivariate analysis DNA-methylation outcome predictor

| | | | | upper | |
|---|---|---|---|---|---|
| N = 96 | exp(coef) | exp(−coef) | lower .95 | .95 | p-value |
| methylation-based | 3.51 | 0.285 | 1.865 | 6.61 | 9.9e−05 |
| predictor | | | | | |
| FLT3.aberration | 1.32 | 0.757 | 0.771 | 2.26 | 3.1e−01 |
| expression based | 1.14 | 0.876 | 0.634 | 2.05 | 6.6e−01 |
| predictor | | | | | |
| cytogenetic | 1.36 | 0.733 | 0.831 | 2.24 | 2.2e−01 |
| risk group | | | | | |

Rsquare = 0.286 (max possible = 0.995)
Likelihood ratio test = 32.3 on 4 df, p = 1.64e−06
Wald test = 28.9 on 4 df, p = 8.18e−06
Score (logrank) test = 33.3 on 4 df, p = 1.02e−06

A combined predictor of gene expression- and methylation-based outcome classes was evaluated. Individuals that were previously assigned to the good prognosis group in both models were assigned to a good outcome class. Individuals that had a poor prognosis in at least one of the models were assigned to the poor outcome class. The resulting model yielded a better segregation into good and poor prognosis groups than either of the models alone. Further, the combined model outperformed all clinical and molecular features currently used for risk stratification (FIG. 9C). The combined predictor also provided significant prognostic information [odds ratio=4.66 (2.27 to 9.58), P<0.001] independent of other parameters in the model. (See Table 7). When the multivariate analysis was repeated on only the subset of samples included in the original test set, the combined predictor was also significant [odds ratio=5.46 (1.74 to 17.15), P=0.004], while FLT3 aberration and cytogenetic risk group were not significant (P=0.068 and 0.620 respectively).

TABLE 7
Multivariate analysis combined DNA-methylation - Expression
outcome predictor In test set samples

| N = 48 | exp(coef) | exp(−coef) | lower .95 | upper .95 | p |
|---|---|---|---|---|---|
| cytogenetic risk | 1.18 | 0.847 | 0.661 | 2.11 | 0.5700 |
| group | | | | | |
| combined | 5.89 | 0.170 | 1.927 | 17.99 | 0.0019 |
| predictor | | | | | |

Rsquare = 0.299 (max possible = 0.99)
Likelihood ratio test = 17.1 on 2 df, p = 0.000196
Wald test = 11.8 on 2 df, p = 0.00272
Score (logrank) test = 14.9 on 2 df, p = 0.000597

Promoter Methylation and Gene Expression

A brief analysis of the correlation between promoter methylation and gene expression was also done. Based on preliminary results, only a minority of genes with variable gene expression shows corresponding promoter methylation. While no such correlation was observed in KIAA1447 so far the relationship between the HOXA10 transcript and its promoter methylation was statistically significant (P<0.001, for the Spearman correlation).

From the results, differential methylation on the long arm of chromosome 17 (17q25.3) was observed. The region of interest is upstream of a the hypothetical protein KIAA1447 and ACTG. This region was found to be a highly variable DNA-methylation region across the analyzed AML samples. The predicted protein sequence for KIAA1447 encompasses a proline rich region with some homology to the forkhead family from Mus musculus. The protein function remains unclear. Interestingly, this genomic region has a high density of CpG islands. It remains unclear if methylation of the examined region regulates expression of KIAA1447 and ACTG; however, the data suggests that gene expression in this genomic area might be generally downregulated by DNA methylation. This mechanism has recently been described by (Frigola et al. Nat. Genet. 2006 May; 38(5):540-9).

The expression of genes in a 1 MB window around ACTG1 was examined for samples with high and low methylation values. A statistically significant reduction in gene expression was found for samples with above median DNA methylation (P<0.05).

TABLE 8

SEQ ID NO

| | | |
|---|---|---|
| 392. | ABO1 | TCAATAGCTAAGTGACATGAAAGCCATAAAAGAAAAAGTGGTCAGCAATATTTAGCAGCACGAC |
| | | GGAGCAGGCGGGTCGGTGGTACTCGCCGTCGGCGCCCAAAGCGGCGGACGCCGGGTACGGCTGCTGATTGGCATTATAAGCG |
| | | AACCCGTTGGCTGCCTGGTAGGGGTAGCCACCGTAGATCGCCGAGCTGTCGTAGTAGGTCGCTTTTTGCATCGCGTTGTTTC |
| | | ACGATCTTGATCGCACACTCTGACAGGGGTTTGACACCCGTGAGGGCGCACATTGGCACGCCCCCGCGGTCACGTGACACTC |
| | | CGCCGCCAATGGCCGCCCCGCGCAGACCTGGTGGGGCGAGAAGCGCAGCGCGGTGAGGGCTCCGCGCAAATCCATCTTACTC |
| | | TCAATAGCTAAGTGACATGAAAGCCATAAAAGAAAAAGTGGTCAGCAATATTTAGCAGCACGACTTGGCCCCGGGCGCAGGG |
| | | AGCCGTGCTATAAAAAACCGCTG |
| 393. | ABCB1 | AGAAAGGTGATACAGAATTGGAGAGGTCGGAGTTTTTGTATTAACTGTATTAAATGCGAATCCC |
| | | GAGAAAATTTCCCTTAACTACGTCCTGTAGTTATATGGATATGAAGACTTATGTGAACTTTGAAAGACGTGTCTACATAAGT |
| | | TGAAATGTCCCCAATGATTCAGCTGATGCGCGTTTCTCTACTTGCCCTTTCTAGAGAGGTGCAACGGAAGCCAGAACATTCC |
| | | TCCTGGAAATTCAACCTGTTTCGCAGTTTCTCGAGGAATCAGCATTCAGTCAATCCGGGCCGGGAGCAGTCATCTGTGGTGA |
| | | GGCTGATTGGCTGGGCAGGAACAGCGCCGGGGCGTGGGCTGAGCACAGCCGCTTCGCTCTCTTTGCCACAGGAAGCCTGAGC |
| | | TCATTCGAGTAGCGGCTCTTCCAAGCTCAAAGAAGCAGAGGCCGCTGTTCGTTTCCTTTAGGTCTTTCCACTAAAGTCGGAG |
| | | TATCTTCTTCCAAAATTTCACGTCTTGGTGGCCGTTCCAAGGAGCGCGAGGTAGGGGCACGCAAAGCTGGGAGCTACTATGG |
| | | GACAGTTCCCAAGTGTCAGGCTTTCAGATTTCCTGAACTTGGTCTTCACGGGAGAAGGGCTTCTTGAGGCGTGGATAGTGTG |
| | | AAGTCCTCTGGCAAGTCCATGGGGACCAAGTGGGGTTAGATCTAGACTCAGGAGCTCCTGGAGCAGCGCCCAAACCGTAGTG |
| | | GCACTGGACC |
| 394. | ACTG1 | GCAGGTGGAGGCAGCTTTGTGGGCCCAGCTGGGGCTGACTCTGCTGGGCTTTTGCCCTCAGGTA |
| | | AAGCCGAACTCCTAACCTCAGGTGCCAAATCCCCCACGGGGGCCTCCGACCACTTCCTGGGCCGCCGTGGCAGCCCCTTGCT |
| | | GAGCTGGTCCGCGGTGGCGCAGACCAAGCGGAAGGCGGTGGCAGCGGCCAGCAAGGGGCCGGGGGTGCTGCAGAACCTCTTC |
| | | CAGCTCAACGGCAGCAGCAAGAAGCTGCGGGCCCGCGAGGCCCTGTTCCCCGTGCACAGCGTGGCCACACCCATATTTGGCA |
| | | ACGGCTTCCGCGCCGACTCCTTCAGCAGCCTGGCCAGCTCCTACGCGCCCTTCGTCGGGGGGACCGGGCCGGGCCTCCCCAG |
| | | GGGAGCCCACAAGCTGCTGCGGGCTAAGAAGGCCGAGAGGGTGGAGGCCGAGAAGGGTGGGCGGCGGCGGGCGGGCGGTGAG |
| | | TTCCTGGTCAAGCTGGACCACGAGGGTGTGACCTCCCCCAAGAACAAGACCTGCAAGGCGTTGCTCATGGGGGACAAGGACT |
| | | TCAGCCCCAAGCTCGGGCGGCCCCTGCCCAGCCCCAGCTATGTGCACCCGGCCCTTGTGGGCAAGGACAAGAAGGGGCGGGC |
| | | ACCCATCCCCCCGCTGCCCATGGGGCTGGCGCTGCGCAAGTACGCGGGCCAGGCAGAGTTCCCGCTGCCCTACGACAGCGAC |
| | | TGCCACAGCTCCTTCTCGGACGAGGACGAGGACGGGCCGGGGCTGGCGGCCGGCGTGCCCTCCCGCTTCCTCGCCCGCCTGT |
| | | CCGTGTCCTCTTCCTCCTCTGGCTCGTCCACCTCCTCCTCCTCAGGCTCCGTGTCCACCTCCAGCCTCTGCTCCTCCGACAA |
| | | CGAGGACTCGTCCTACAGCTCAGACGACGAGGACCCGGCTCTGCTGCTGCAGACCTGCCTCACCCACCCCGTGCCCACCCTC |
| | | CTGGCCCAGCCCGAGGCCCTGCGCTCCAAGGGCAGCGGCCCTCACGCGCATGCCCAGCGCTGCTTCCTGTCCAGGGCCACGG |
| | | TGGCTGGCACCGGTGCGGGCTCAGGCCCCAGCAGCAGCAGCAAATCCAAGCTCAAGCGCAAAGAGGCCCTGAGCTTCTCCAA |
| | | AGCCAAAGAGCTCTCCCGGAGGCAGCGGCCGCCCTCCGTGGAAAACCGGCCAAAGATCTCAGCCTTCCTGCCCGCCCGGCAG |
| | | CTCTGGAAGTGGTCGGGGAATCCCACACAGGTAGGTCCAGCGGGAGGCGGGAGGAGCTCCTGGTTCCCAAGGAAACCGGGGC |
| | | GGGCTCATGCGCCCCTGCTGCCCTTCCCTCTCCTTTTTCATCTTCCTACTTGATTTCAAGTTAAAAAATGTGGAAAACTCAA |
| | | GGGAAGAACAAAGACCCATCCATGACCCA |
| 395. | ACTG1.01 | CTCCAGGCTGGGCGACAGAGCAAGATCCTGTCTCAAAATTTGAAACAACTTCAAAGAAGGAAAG |
| | | GAAGCTATGCCTGTTGCATACAAGCAGAATCTTCCTCGCGGCTCGGCCTGCTCCAGCCTCCCGGAGGATGAGCCTCTGACCA |
| | | GGGCCGGCCCAGGTGCCACAGTCACAGCCAGTGGCCCGCAGTTCTGAGGCCCCTGGCACCGCTGAGGCGTCCCTGGAGCTTC |
| | | CTCACCAGCCTCACGCATCCTGCCGGGGTGGCGGGGTCAGGGCCCACCTCTGGGCACGGGAGGCGTGGGCCTCACGGGCCAC |
| | | GGATGGGAGCCCAGGGCAGGGGGCTGGAGCCCTCGGGGGTCAGAGCCCGTGCGCCTTCCAGGGCGGAGGCGGTCAGCCGATG |
| | | GACCAGGCGGTGCCGCAGCGACAGAGGGGGTTGAGATCGGGGCTTTCCCCTGCGACCAGCTCGCAGCCTGACGTCGGCCTCG |
| | | CTCTGGGGAAACCAAGGCACGGGCACGCGCGGGCGTGGCGCCAACGTCCTCGCTGCCGGCCCCGCCTCGCCCGGGACAGCGC |
| | | CCCGGCCCTGCTCCCCGCTCTACGGCCCCGCGCCCCGGCCATCTGCGGCTCCTCTTGCTTCGCGCCCGCGCCGCGCTCTGCC |
| | | CACGCCCAGCCCCGCAGCTTCTCTGTGACCCCCGCCTGCCGCACCCCGGCTAGGGGCTTATTAGCGCCACGGAGCTGAGCTG |
| | | GACGTGTCCAGGATCTGGGGAGGGAGCAGCCCAGAGAGGGCAGGGCCTGGCCAGGCCTCCCCAGGCAGCCCTGCGGTCCTGC |
| | | AGCCACGAGGGGAGGGGACGCCACAAATCCGGGTGCCGACCCGGGCCAGGAGAGTCGGGAAATGGGCCTTGGCTGGGACCCA |
| | | GGCCGACTCTGATCCCGCGGGCCTCGTAGCCCCTCCAGCCCT |
| 396. | ACTG1.01 | TAGGGGACAGAGCCCTCCCTTAGTGATGCTGTGTCACCGAGGATGTAAGAGTAGAAACCTTTAG |
| | | CTCACAACACCTACCCAGGAAGGAAGGCTGGAACAGCGCCTCCGGACACCGGAACCGCTCATTGCCAATGGTGATGACCTGG |
| | | CCATCGGGCAGCTCGTAGCTCTTCTCCAGAGAAGAGGAGGATGCGGCGGTGGCCATCTCCTGCTCGAAGTCCAGGGCGACGT |
| | | AGCACAGCTTCTCCTTGATGTCGCGCACGATTTCCCGCTCGGCCGTGGTGGTGAAGCTGTAGCCTCGCTCAGTGAGGATCTT |
| | | CATGAGGTAGTCGGTCAGGTCCCGGCCAGCCAGGTCCAGACGCAGGATGGCGTGGGGGAGGGCGTAGCCCTCGTAGATGGGC |
| | | ACCGTGTGGGTGACCCCGTCTCCAGAGTCCATGACAATGCCAGTGGTGCGCCCAGAGGCGTAGAGGGACAGCACGGCCTGGA |
| | | TGGCCACGTACATGGCCGGGGTGTTGAAGGTCTCAAACATAATCTGAGAAGGGACAAGGGGCGGCTTAGTCAGGGACAGAGA |
| | | CCCACGGCCACCCCATGCTCACACGCCACAACATGCTGCATGCCAGTGTGATGTGTGGAGAAAAGAGAACGCAGGCAGAAAC |
| | | CAAATGAGAAACCTGGAGGCTTCAGGGAGGAAATGCCGGGAGAGGAACAGAGCCTGGAACAGCGAAGAAACACTTAAATGTC |
| | | AGAAATCAAGCCGGGCAGAAAATGACTGGGGAAAGGACGGGAGGAGCACGGGCGTCGGCCGAGCCTCACCTGAGTCATCTTC |
| | | TCTCTGTTGGCCTTGGGGTTCAGGGGGGCCTCGGTCAGCAGCACTGGGTGCTCCTCCGGGGCCACGCGCAGCTCGTTGTAGA |
| | | AGGTGTGGTGCCAGATCTTCTCCATGTCGTCCCAGTTGGTGACGATGCCATGCTCAATGGGGTACTTCAGGGTCAGGATGCC |
| | | ACGCTTGCTCTGGGCCTCGTCGCCCACGTAGGAGTCCTTCTGGCCCATGCCCACCATGACGCCCTGCAGGGGACGACCCGTC |
| | | AGCCTCGCCGGCGACACCGAACCCACCCCGCAACGCAGAACCCAGGAGCCCCGCGGCGCCATCCACTCACCTGGTGTCTGGG |
| | | GCGCCCGACGATGGAAGGAAACACGGCTCGGGGAGCGTCGTCCCCAGCAAAACCAGCTTTGCACATGCCGGAGCCATTGTCA |
| | | ATGACCAGCGCGGCGATCTCTTCTTCCATTGCGACCTGCCCGGAAAAGGATGGACTCAGGCGGGCGCGTCTGTAACACGGTC |
| | | CCCTCCCCACAGCCACCTAATGCCCTCCCGCGGGGAAGCCTCGGCCCTGCCCCAACCCCAGCGGCCGTGGCCTCCAAGATCG |
| | | CAACCGCCTGGAACCGAAGGCCGGGCCTTTTACGTAACGTCCACGGCTCGGAAGTCTGCACTGCGGCCGGGCCCCGCCCTGG |
| | | ACCCCCGGCGCCCCCCCAGCCCCGTCCGCCTGACCCGGCCCACCCCGCCTTTTGTTCCCGGGGAAGGCGCGACGAGGCCTCG |
| | | GCAGCTGGAAGCGGGGCCAGCCGGGGTCGGGGGGCGCAGGCCTGCGACGTCCAGCTCAGGCCCCGGGGCGGGGCGCTCACCG |
| | | GCAGAGAAACGCGACGGCGGAGCGGCGGAAGAACAGAGTGCGAGAGCTGGCAGCGGCGACTGAGACCGACCGCGGCCTCCCC |
| | | CGCCGTTATTTAAGCGGAAGCGGCGCGCCCGCGCGGCCCGGCGGCGCGCGCGGCCCCAGAACATGTCCATATATGGCGATCT |
| | | TTCCGAAAGCCGGGCACCCATTGGCCCGCCGGGCAGGGACACGTGGGGCCCCGGCCCGCCCCGCCCCCAGCCCGCACCGCCT |
| | | CCCAACGGCCCCGCCCGCGCCACCCCCGCGCGCTCGCGACCCTGCGCGGGCCGGCGGGCGGGTCTGAGTTGGGGCGCCCTCC |
| | | GAGGGTCGCCGGGAGGGCCGAAGGGCTGACGGGCCTGGCCCCTCCCCGGGACTGCCGCGCCGTGGGGAGGGCCCTGCTGCGC |
| | | CCCGAAACTGCCTGACCCGGGGCGGGGCCGCGCCGGAGCTGGGGTGGGTCCCCGAGTCCCCGGCCACGCTGCGGGGCTTTGC |
| | | TCCCTGGGACGTCCCTTGCAATCTTTCCCCTCGGGCTCCACGAGGATCTCGGGCGCCAGTGCCGGGCTGAGGAAGGGGCGCG |
| | | GCGGGGGTCCGCGACGCTCTGCGTCCCTGGGCGCGGGGGAGCTCTCAGGAGCCCTCACCTCGCCCCGGTCACCATAGGAAAC |
| | | AGCCCGCCCGTCCCCAAGCGCAGCCACCACCATGCAGGTCCCTCCACGGGGCGCCCTGCCCCGGATCTCCGTTCGTTTGAAG |
| | | ACCGAGGCCCCTTTCTACCCGACCCGACACATCCCTGCCCCTCAAATCACAGGCCTGATTGTCCCCAGGCGCGCGCTCCGGG |
| | | TTGGGGCTACCCCGCCCCCTTATGTGCTGAGAAGGTGGTGTGGAGTGAGGCCTTTGTTTCATTCTGGTCACCTGTAGACATT |
| | | ATAGGGAAACTGAGGTAGACAATGGGAGCCCTGACTTTTTTGAAGGTCGGCTCCCCAGCTTCGGGCGGTCGGGGGGATACCC |
| | | TCTTCTGTTTACGATGCTAGTTAAGAGCAGCGATCAGGGCTGGCGTGGGTTTAGCCACAGGCTTGTCCAGGGCGCTCACTCC |
| | | TCGCCAGGAGCGGCGCCGGTGCTTTGGTTACACCCCGAGAGGCGCCCGGCACTACGGTTTCCTTGTGCGCTGCTGGAATTTT |
| | | CAGGGTTGGAGCGCCGGCCGCGTAGGGGTGGCAGTGAGGCCGCGCAGTGAGGGGACGGACTCGCCCGAGGTCGCAGAGCGTT |
| | | CAGATTGACTCGACAGGCTGGGGGGCGGGGCCGGGGTCCCCACGCGGAGTCCGGGGCCCTGCAACGTTAGCTCGCGACCCCG |
| | | TGTTTGGGAGTGACAACAGCGAGTCAGGGCAAGGCGGCCGGCCCAGCTCACCTGCGGGCTGCAGTCAGCCCGGACGCCGAGA |
| | | CCCGTGCCTAAGGCCCCCGTGCCCCAGGCCTCCCAGGGGTGCTGAGCGCCCCCAGGCCCAGAATCTCCGGGGGCCCGGGCAA |
| | | GGCTGTCAGGTATGTTCCCTCCCGTGGCGAAGCCGGGGCTGGGCCTCCAACAGACCCACCCGGACTCGCGTCCCTGGGCAGA |
| | | ACCACACAGCCTGCTCGACCCCCGGGTGAGGACTTCCACTTGGGGGGTCCCAAGATGCGACGTCACACACCGAGGGACACGA |
| | | GGCGGGTGGGGAATAGCCACAAGCCCCTCCATCCTGCGTTCACCCCACACTACGTTCTGCTCCGGGCCGGCCGGGGACCCTG |
| | | CCCCTCCGGGTCTCTGGTCTCCTCCGAAGCTTCTTTGTCTAGGAAGTGCGGGGAGGTGCCGGCCCCGGGCGCGCGTCCCCGT |
| | | CCCCCACCTTTCTTCCCCACGTTGCGGACCCGAGGCCGCCAGGGTCGTGGGGGCGGGGGCGAGGCTGGCGCCTCGAGACCCC |
| | | CGGGTTCCCCGCCACCCGCAGGGCCTGAGCCGCGGGGAGGGGCGGGGAGGGGACGCCGCCGCGGGCCGGGGGTGGAGAGCGA |
| | | CTTCCTCCTCCGCGGCCGCGTCACGCGGCAGGGGTCAAGGTGTGGCCCAGGTACGCGGCGGGGGGCGGTGGCGCGGCCATAT |
| | | TTGGCGACTTCGGCGCCGCCGGAGCCCCGCCCGCCTCCGCCGGGAAGCCCGGCCCGGGGCAGCGCCATCCCCGCCCGCCGCC |
| | | GGGCCTGAGTCACGGCGGGCGGGGGGCGGAAGTAACGGACGGGGGGCGCCCGCCCGGCGCTCCTGGCCTTATTTAGTCAAGG |
| | | AGGCCGCCGATTGGCGCGCGGGACAGAGGGGCGGCGGCCGGGCAGCCTCGGGGCGTAGGAGTGGCCCGGCGCCTCCGAGGGG |
| | | TCGGGGCGGGAGCCGGGCGTGGGGGGCCTCGGAGCCTGGGGAGTCCCGGGACCGACGGCGGCGGGGATGCGGGTCTGCCTGT |
| | | CCCTGGGATGCGGGCGGGCGGGCAGGGCGAGGCCTGGAGCCCGCGGGGCGCAGGGGGCTGGACCGCGCGCTGTGATTTTTCT |
| | | GTTTAGCAGCGGGGCCTCGCCCAAGGTCGCGCGGGGCCTGTCCTGGATATGCTCTGGGAAGCCAGTGGCGCAGGGCAGCCGC |
| | | CACCCTTCTGGAACCAGTGCCGGAGAGGGCCGGGCAGCTGGGCCTGGGGAGGGGACCGCATGGATGGAGCTGGGGGTAGCTG |
| | | GGCGGCCTCCGGAGCCGGGAGAGTCGGCAGCTGCACTTCCGCCAGAGGTGGGTGTGTCCTTCACATTTCAGGAAGGGAGACT |
| | | TGGGGCCTGGAGAAGCGATGTGATTTTTCTTTTCTAGTTCAGTGCTGGTTTTGATGGCT |
| 397. | ACTG1.02 | GGAGGAGGGAGCGCAGAGGAAGGCTAGGCCGATTCGCAGCTGCCTCCTATCTCAAAAACGTGGA |
| | | AGGGGCTATAACTCTAGGGCTGGGAGGGAGGGGAGTCGTTTTCGCTGACTCTCCGCACGGACCCTCTCCTGCCCGCTAGCTG |
| | | GTCAGCTCCTCCAGCTCTCCCCGCGGCAGCAGGCCGGGGAGCAGATGTCTTGTCCAGTGTGCGCGGGGCACCGAGGTAGGGA |
| | | AAGCCTGTTTAACCCGTCCCGCCCCTCCTGCTCTGGCCAGCAGCTCGCCCAAGCAGCTCTGCAGAGCGACTAGGTCAGGGCC |
| | | TCTCCAGGTGTCTCCACTCGAGGGACGGCTGGCGCCACGCGGAAAAGGGGAAGCGGGTCAGGGCGGGACAAGGGCAAGAGGT |
| | | TACTGCTCTGGGCGCTGTGTCCTAAGGGCTGCCTCTTGTCTGCGGAGAATGGAGGGTGCCGGGGGTCAAGCCCGGACTCTGT |
| | | CAGGGTACCCGAGTCTTAGCCACGCCATCCTTCCCCAGCGCGCCGCGGGGGCGGGCACAGCAGGGCGGGCGTGGCTGGGAGA |
| | | CCGGCTGTGCCTCGCCGCCCCGCCCGTCCCAGGCCGGGAAGCTGGGACTTGAGCTGGTCTGCATTCGGGCAAGGTGAGCCCC |
| | | GGGATGTTTCTCAGGCAGGGCCCAGGGCTGCACGCCCTAACTTGGTGCGGGGTCGCGGCGGTCCAGCCCGGCTGCACGTGGT |
| | | CGCGCGCTCCTTGCAGCTCCCAGCCCGCCGGCCCGCGCTCCTGCGCCCCCTCCCCACGCAGTCGACCTCTCCTGACCGCCAG |
| | | GTGCACGCAGGGCGCGGGCCTGGGCCTCAGCTCCGCGCACTCCCCCCGGGACGCGGCACCGGGGCTTCGGGTGGGCCCAGGA |
| | | CCCGCACTCAGCACGCACTTCCCCCGCCGAGCACTGTCTGGCAGCGGGGACGCACCCCCAGGACCACGAGCTCGGAGGCCTG |
| | | GGGCCGGGAGATGGCGCCCGTGGAGTGGGTCCCGCGAGAAGGCCGGGAATGGCGGGCCGCTGTCGTTCCCACCTGGGCCGCC |
| | | AGGTGGGTGCCGCTATCGCCGCGAGGCCGCACCTGTCGCGCTGCCGGAGCCCGCGCCTGCCCAGGAGCGCCAGGAGGGACCC |
| | | CCGCTCCTCCCTCCGCCACTGGGCGGGGCCGGTCGCTGAGTCACGGCCGCACACCGGACGGTGACAGAGGCAGGGCTGCCTG |
| | | GGGAGGGGCCGGTGACCTCAGTCAGGGCTGACGCGGAAAAGTGGGATGGGGTCACCCGTGGCAGTGGATGAGGGGGTGGCCC |
| | | GGGCCTCCACTCACTGCCCACGCTAGATGTCCGGGGTCCCTCCTGCGACCGAGGACAGGAGGCACCCACAGAGGACCCCAAC |
| | | ACGAGGACCCTGGCCCTGGGTGCGGGGCGTTTTTCCCGCACTCTGCTCCCCTAGCAGATGGCCCCTGCTAAGCACCAGCTCC |
| | | CAGAAAAGGAAAGGAAGGGGCTGAGAGGCTGGATCGAGGCAACCCAGCAGGAAAAG |
| 398. | ACTG1.02 | GATCCTCCCGCCTCGGCCTCCCAAGTGGCTGGGACCACAGGCGCGGGCCCCCTCCAGCCAAGCA |
| | | TTGTTTTTCTGACCCGCAGGATGGGAGCAGGGAGAGCGTGGCTGGCCGGGGGTCCACCGGGAGGCCCTGCGAAGGGCAGGCC |
| | | CCGGCCAGGCGGGGCGCGGCTCCACGTCTCAGCCGGCGCTTCTGCGCACGGGCTCACTACTGGGGGCGGCCGGGATCACGGA |
| | | CTCGCTGGCCTCGTCTCCGGAGCGGAGGCGGGCAGCTTGAAGGAGCGACCCCGTGGGCCGGCGCATCCCTCCCCAGGCCCCG |
| | | AGACCCGGCGGGAGACGGACCCTCCCCATGCCTCGCCGCGGAGGCCCGCGCCGAGCCAGGAGCCGCGCATCCATTGGCCGAG |
| | | AGCGCGGCCGCCCGGGCCAATCACAGGGCGGCCCCGGCGCCCCCGGGCGCGCCGTGGGGACAGAGTCAGGCGCGTGCGCACT |
| | | CGGCCCTCCCCGGCGGCCTCCAGGCGGGACGCGGCGTCGGCGCCTGAAGTTGGGGCTCCGTCCTGCCTCCCTGCGGGCCGGG |
| | | AAAAGGGTCTCGCACGTGCGCCCCCCGCGGTCCGCGATCCCCAGCCGTCGGCCTAGCCCCAGGCGGGTCGATCCCGCTGTCC |
| | | CCAGCCCCGGACCAGCCTCCTCCAGCTGCCGGGTGGAGAGGCTGGGGGGCTTTTCCCGAGGTCGGCGGCGAGGCTGAGAGCC |
| | | CCGGCCCCGCCGCGCCCGAGGAGACAGCCCTTCGCGGGCTCTAAAGGCCCCGGGGCCTCCGGGATGTTCCGGGGCCGAGTTG |
| | | TGCCTGTTGCCGTTTCCGGCGCCCGGGGCCTCCGGGATGTTCCGGGGCCGAGTTGTGCCTGTTGCCGTTTCCGGCGCCGCTG |
| | | CCCTCGGGCACCGTCCCCTCTGGCCCTGCCTTGGTCTGGGAGGGGTCGCTAAGGGGGCGGAGGGCGCTCCTAGGGGGCCCTG |
| | | CGGATCTCCTGGCCTCGGCCCCCCCGGGTGGCCAGAGCGGGGCTCTGCACAAAGGCCTCAGGTAGCCGCGGCCTCGGTTCCG |
| | | AGAAAGCCCCTCGCACTGACCGGGCCTCAGCTGACCCGGGCAGCCTACTGACCCGACCCTGACGCCCGGGCCCTCCAGGCCT |
| | | GTCAGAGCCTCCAGGAAAGGAAATGGGCTGCGGGGCGCAGCGAGGCTGGACAGCGGGCGCTGGCCCAGGACTCCCCTTGCCC |
| | | AGAGGAGAAAATGGAGGCTGAGCCTCCGGAGAGCCCAGCTCGATGCCAGGAGCACTTTGCAGAGGGACACGGATGGG |
| 399. | ACTG1.03 | GGTGGAGGCGACCTGGGCTGCAGGTTGTGGCTGGGGTCCTTGCTATGCTTCTGGCAGCAGGAAT |
| | | CAGAGAAGCAGTAACTGGATCCAGCTGAGGGCCTGGCGGCCACCGAGGGTACGGGCGGCGATCAGGTTGCGGCCGAGCCGTG |
| | | AGCGTTCCACTGGACGGCTTCTACATCCGGAAGGGCTGGGCCGGGCCGGGCCGGGAGCCCACACGGGGTTGGAGGAGGGGGG |
| | | CAGGAACGAGAGAGGCCAAAAGGTGCAGGGCCGTGAAGAGGGGCGCTGGGCCCACGCTATGAGGAAAAGGGGATCAACCCTG |
| | | CCCTCCAGCCCCGTAGGCCCTCAGGCCCTCGGGCTCTGGGCTCTGTGCAATCCGGGAGTGGCTGAAATCCAAGCTGAGGGTA |
| | | ATGAAAGGTGCTGCTCCTAAGCCGCACGTCTCTGATCCGCCCTCCCCGCCCGCCCTCTGCTGCCCGCGGGCCCTCTAAGAGC |
| | | TGCCCAGGCTGCTGCCGCGGGTCAGAGGCGGGTCAGAGCAGGCAGGGGGTTCGTGACGCCGGCTGGGTCTGGGGGCTGTGGG |
| | | CCAGCCGAGCCGACCCGGGCTTCTGGGGGACCGCGGGGGCCGTGAGCACTCAGAGGGCGCATCCCAGGCCCCTCCGGGGACC |
| | | CGGCCAGCCTGAAGATGCCGACGAACGGCCTGCACCAGGTGCTGAAGATCCAGTTTGGCCTCGTCAACGACACTGACCGCTA |
| | | CCTGACAGCTGAGAGCTTCGGCTTCAAGGTCAATGCCTCGGCACCCAGCCTCAAGAGGAAGCAGACCTGGGTGCTGGAACCC |
| | | GACCCAGGACAAGGCACGGCTGTGCTGCTCCGCAGCAGCCACCTGGGCCGCTACCTGTCGGCAGAAGAGGACGGGCGCGTGG |
| | | CCTGTGAGGCAGAGCAGCCGGGCCGTGACTGCCGCTTCCTGGTCCTGCCGCAGCCAGATGGGCGCTGGGTGCTGCGGTCCGA |
| | | GCCGCACGGCCGCTTCTTCGGAGGCACCGAGGACCAGCTGTCCTGCTTCGCCACAGCCGTTTCCCCGGCCGAGCTGTGGACC |
| | | GTGCACCTGGCCATCCACCCGCAGGCCCACCTGCTGAGCGTGAGCCGGCGGCGCTACGTGCACCTGTGCCCGCGGGAGGACG |
| | | AGATGGCCGCAGACGGAGACAAGCCCTGGGGCGTGGACGCCCTCCTCACCCTCATCTTCCGGAGCCGACGGTACTGCCTCAA |
| | | GTCCTGTGACAGCCGCTACCTGCGCAGCGACGGCCGTCTGGTCTGGGAGCCTGAGCCCCGTGCCTGCTACACGCTGGAGTTC |
| | | AAGGCGGGCAAGCTGGCCTTCAAGGACTGCGACGGCCACTACCTGGCACCCGTGGGGCCCGCAGGCACCCTCAAGGCCGGCC |
| | | GAAACACGCGACCTGGCAAGGATGAGCTCTTTGATCTGGAGGAGAGTCACCCACAGGTGGTGCTGGTGGCTGCCAACCACCG |
| | | CTACGTCTCTGTGCGGCAAGGTAGGGAG |
| 400. | ACTG1.06 | GCAGGTGGAGGCAGCTTTGTGGGCCCAGCTGGGGCTGACTCTGCTGGGCTTTTGCCCTCAGGTA |
| | | AAGCCGAACTCCTAACCTCAGGTGCCAAATCCCCCACGGGGGCCTCCGACCACTTCCTGGGCCGCCGTGGCAGCCCCTTGCT |
| | | GAGCTGGTCCGCGGTGGCGCAGACCAAGCGGAAGGCGGTGGCAGCGGCCAGCAAGGGGCCGGGGGTGCTGCAGAACCTCTTC |
| | | CAGCTCAACGGCAGCAGCAAGAAGCTGCGGGCCCGCGAGGCCCTGTTCCCCGTGCACAGCGTGGCCACACCCATATTTGGCA |
| | | ACGGCTTCCGCGCCGACTCCTTCAGCAGCCTGGCCAGCTCCTACGCGCCCTTCGTCGGGGGGACCGGGCCGGGCCTCCCCAG |
| | | GGGAGCCCACAAGCTGCTGCGGGCTAAGAAGGCCGAGAGGGTGGAGGCCGAGAAGGGTGGGCGGCGGCGGGCGGGCGGTGAG |
| | | TTCCTGGTCAAGCTGGACCACGAGGGTGTGACCTCCCCCAAGAACAAGACCTGCAAGGCGTTGCTCATGGGGGACAAGGACT |
| | | TCAGCCCCAAGCTCGGGCGGCCCCTGCCCAGCCCCAGCTATGTGCACCCGGCCCTTGTGGGCAAGGACAAGAAGGGGCGGGC |
| | | ACCCATCCCCCCGCTGCCCATGGGGCTGGCGCTGCGCAAGTACGCGGGCCAGGCAGAGTTCCCGCTGCCCTACGACAGCGAC |
| | | TGCCACAGCTCCTTCTCGGACGAGGACGAGGACGGGCCGGGGCTGGCGGCCGGCGTGCCCTCCCGCTTCCTCGCCCGCCTGT |
| | | CCGTGTCCTCTTCCTCCTCTGGCTCGTCCACCTCCTCCTCCTCAGGCTCCGTGTCCACCTCCAGCCTCTGCTCCTCCGACAA |
| | | CGAGGACTCGTCCTACAGCTCAGACGACGAGGACCCGGCTCTGCTGCTGCAGACCTGCCTCACCCACCCCGTGCCCACCCTC |
| | | CTGGCCCAGCCCGAGGCCCTGCGCTCCAAGGGCAGCGGCCCTCACGCGCATGCCCAGCGCTGCTTCCTGTCCAGGGCCACGG |
| | | TGGCTGGCACCGGTGCGGGCTCAGGCCCCAGCAGCAGCAGCAAATCCAAGCTCAAGCGCAAAGAGGCCCTGAGCTTCTCCAA |
| | | AGCCAAAGAGCTCTCCCGGAGGCAGCGGCCGCCCTCCGTGGAAAACCGGCCAAAGATCTCAGCCTTCCTGCCCGCCCGGCAG |
| | | CTCTGGAAGTGGTCGGGGAATCCCACACAGGTAGGTCCAGCGGGAGGCGGGAGGAGCTCCTGGTTCCCAAGGAAACCGGGGC |
| | | GGGCTCATGCGCCCCTGCTGCCCTTCCCTCTCCTTTTTCATCTTCCTACTTGATTTCAAGTTAAAAAATGTGGAAAACTCAA |
| | | GGGAAGAACAAAGACCCATCCATGACCCA |
| 401. | ACTG1.09 | CCCAGGGGCCCCAAGGACAGCCGGACAACAGTGGGCGGGTCCAGAGCACCTGGGACACCCGAGG |
| | | GCCAGGTGTCTGCTTCGTCCACCCTGGGCCTTGGTCCGGGCACTCCCTGCGGTCCAGTGCTCCCGGGAAAGCGCCCGCTTCC |
| | | TGCCGTCTTCCCTGTGGGCCCCAGAGAAAGCCCCGGGGTGGAGGGCGTCCCCCAGCCGGGCACGGCGCCGTGGTTGTTGATA |
| | | GCACGAAGCTGACAGTCTTCAGGGCTCGGCCGCTGCTTTCTGGATGCATAAAGCTCACCACCGCGATGAAGAGAATGTTCTC |
| | | GAACAGTGCATTTCCCCAGGGGCCCGTGGTCACCCTGTGCTGGATGAGGCTGCTGGCCCGCACGCGGCAGGGGTGGTTCCGC |
| | | AGTGCTGGGAGCTGCCTGCCCTCGGGGGTATCTGGCCCAGAGTCCCTGCTTCTGGGTGGGGCTGTGCAGAGGGTTTGAGGAA |
| | | GCTTGGAGTCCTACCAG |
| 402. | ADFP | AGTGAGCCTAGATGGCGTCACTGCACTCCAGCCTGGGCGACAGAGCGAGACTCCGTCAAAAAAA |
| | | AAATGCGGTTTTCTAACGCGTTTCCCTTTCGATAATGTCCCTTCGACAAATATTGTTTTCCCACAACCACCCAAGCAGGCCA |
| | | AGGGACATTCGATAGCAATCGCCCTAAATCTGTTGGCAGGAAAGAAGCCTCAACGCACAAAGGCAAGGGTCGAAAGCGCGGG |
| | | TTCAGCAGACCGCCCCTACCCAGCCAGGACCTGGAAGCCGCGAGGCGAGCGGGGGGTCTCGGACCATCACCCCCGCGTCCCG |
| | | TCCACCTTTGAGCCCCGCGGGGAGCAGAGCAGCGGTCCCAAGGCCCCGGGGAGCCCCCCACCCCAACTGGGCGCCGCTGGGG |
| | | GCCCGGGACCGGTTCGCTGGGGCACCCACTGGGCGCGCACTCACCGACGGACTGCAGCGAAAGGCGAAGAGCAGGCGCGTCC |
| | | CGAAGACGACTCCGGCTGCCACGACCCGGTCAAAGCGTGGGGCCCGCGGGCGCCCGGGCTATAAACCCCGCCTCGCCGCCCC |
| | | TCCCCAAGCCTCCGCAGCACGTGACCCGCGGCAGCACGCCCTGTCCCAAGCCCGAGTGTCACCCTCGGGCACGTCCCGGCGC |
| | | CCTCCCCCAACGCCGGCCCTCCTAGAGTGGTGGGGGCCCGGTCGGGTTGAGAGGGTGTGCTGCCTGGGCGAGGGGCCCCGCC |
| | | ACGGGGAGAGGAGCCTGTCGGGAGCCGGGCGGGGGCCCGGCCTGGGGAGGCTGGGAGTTGGAGGTAGCCCTGCCTGGAGTCG |
| | | GGGCGCTGCCTGGAGTCGGGGCGCTGCCTGTGGTCGGGGTCCTGCCTGTGGTCGGGGTCCTGAGAGGCCCGGGGCAAATGGG |
| | | GATGGCGCCCAGGCCGGCTTTGGGGTTTTGAAAAATGCAAACAAATTCACACCAACTTGTTGAACGGCGTTAGTCCTCAGAA |
| | | TAGAACTCTAAAAATGGAAGCGGTGGCTCACGCCTGTAATCACAACACTTTGGGATGCTGAGGCGAGAGGATCTGTCGAGCC |
| | | TAGGAGTTCGAGACCAGCCTGGCGAAGATTTTTATTTTATTTTAAAAAATAG |
| 403. | AFP | AGGTCGCAGACTCGCACCATCCCACGAGGAGACGAGGCCGAGCGAGCTCTGCTGCTGCCGCCAA |
| | | CGCCGCCGCCGACCTCCGCCGCCGCGGGGGGGCCCGCCACAGCCGCCACCGCCGGGGGGCTCCCTTCTCCTTCTGCAGCCGT |
| | | CGCCGCCGCCACCGGAACCGTCGCCTTCTCCATCCCCAGGGAAAGAGGGAGGGCGCGGACGGGGGAGGGGCGTGGGGCTACG |
| | | CTCTACCGCGACTTCGGCCGCACTGGGGCCGACACAGCAATCGGTGCCGCCTCCGCCGCCACCGCCTCGGTCACCTCTACTG |
| | | CCGCCGCCGCCGCCGCCGCTCCGCCAGCTCTCACCTCGTCTCGCGATACTAAGGGCGGGGAGCCTGACGATGGGAGGGAGGG |
| | | AGGGAGGGAGAGGGAGGGAGCAAGGGAAGGAGGGTGAGGCGTAACAGGGAGACCGGGTGTCGGGAGAACCAGAGGAGAGGAG |
| | | ACTGGAAAGAAGCGGGGAAGTGTGCCTGGGGCAGCTTCCGAAGGGGAACGGCGGGGACAGATGAGGGGGGCGCGATTGCAAC |
| | | CAGAGCGCCCGCGAGCTCCCGGATTTCTTCTCTTTCCTCGCTATTTCCCGCACACACAGCCCTGCAGCCCTCCCTTCTTCCT |
| | | TCCTGGCTTGTGATTAGAACGCAGCCGCAGGCGGAAGTGCAGTGACGCCATCAGCCGTCGCCTGAGCCGTGGCGCGCGGGTG |
| | | GCCGAAAGGCGAAAGTGAGATTTCAAGGGCCGGGTCTGGGGCCCCGCGGGCGCGCAGTGCTGCGGCGTGTGGGGAGTACGGG |
| | | GAGGCTGGAGAGGAGCCGGCCCCGGGCAGTGTGAGGTGCGGAAGCGCGGTCCAGACACTTTGCTGACGGGAGGCCGGTCCGC |
| | | CAGATCCGGGAGGTGGGGCCTCGTTCTGGACTGCTGTACGTAGTTTTATTCCTGGTCATTTTTTTCCACCGCCAAATCGAGA |
| | | GATAGAGCTTTGGATCATGAGAGATGGCGTCGTGTTGGGAAAAGAGCCTTCTTTACCTGAGTTTAAGCTCGGCTCATAGACA |
| | | TCCTCTACCTGAGTTTACACGTGTGTAATATGAAGGCAGTCGATTAGGAGATGTCTAAGATCATTACTTACACCTC |
| 404. | AGT | CCTCCTCAGCCCCTCCATCCCGCCCTGAGAACCAAAGGGCCAACTTCTGGCCATGCATAACGAAGCAGAGCAGTC |
| | | TCAGGGTAGAAGGCACTGGAAAAGCGTTGTAGGACAATTTTTCCCAGTAGAAACGGTGGGGGTGGGAGGGAGACCCGCTCAC |
| | | CCCTTTTGACCCACCCCTGCAGCTCAGCCACGCCTCCTAGGGAGGTGGGCAGCCTTGCAGGAGCCCGCGCGAGGCTGCAGTA |
| | | CCGCCCTCCGCCACATGAGGGCAGCACGGCCTACCCGTGCAGCAGCGGCGGCCCCCGCGCCCCTCTTGGCCAGAGCCAGGCG |
| | | CAGGCCGGCTTGCGGTGCCGTCGTTCATTGGCCGCGGCGCGGGCGCTGCCATGTTGGCGGAAGCGGACCCCCCTGTGCCGTG |
| | | GAAACTGGCGGTGGCCGCGGCCGCCGAGTCGGTCTGCGCAGCCTCCTGCGTTTTCTCGCTTGGATCTTGGCACTGAGAGGCG |
| | | GTGGCCGGCGGGATGGAGAAAAGTAGGATGAACCTGCCCAAGGGGCCGGACACGCTCTGCTTCGACAAGGACGAGTTCATGA |
| | | AGGTGCGCGCGGCGTCCGCTCCCCGGAGCCGGGCCATGAGGGTGCCTCTGCCCTGTGCCCTCTGTGGCGGTCTCTTCGGTCG |
| | | GCTGCTCCTGCCTGCGCACGCTCAGTGTCAGGTCCTCCGCCGAGACCTCGCTGCACTTCGCAATGAGTCTTTTGGCGTCAGG |
| | | TTTGGCGGGTGCAGCCTGGAGAAAGCACTTCTGTAAAATCGTGCGTGTTACATGGAAAGGCCTGAAAGTTCTGGGCAAGTGG |
| | | TTTTGAGCTGCATGTCAAAGCCACTGTTACCCGTTCCTGTTACCATGCAGTGCAGTCAGATTGCCAGTCCCCTTGTCGGAAA |
| | | GAAGCTTGTGGACCGGGGCCTTGGAGCTCATTAAGTGTGTGTT |
| 405. | AMIGO2 | TCTCACCCACCATCAGAAACTCAATTTTTTCCTAAGGTGCATCCTGCGTTGTTGTTGTTGTTGT |
| | | TGTTGTTTTAAACTCCCTCCAACAGACACAACTATACGCAAACGTACGCTACAAACAATTAAACGGAAATACTTAGCAACCA |
| | | CCCATCCAGGAAAAAACTCACGGTGCTGGGGAGCCTCGTGGGCTCCGGGGAGGCTGCCTACGCAGTGCCTTCCGAAGGTCTG |
| | | CGCGTCCGTCTGTCCGTGTCTGTCACTCTTGCACCTTCCGACTTCCTTTCCCTCCGGCTCCCGGCGGGCGGCACCCTCTAGC |
| | | CGGCTCTCAACTTTGAGGAGTTTCAAGCAGCCGCGGCGGCAGCAGCAGCCCTGGACGCAGCAGCCAAGCTCTTCGCTGGCTC |
| | | CCGGGGGCTCTCCCGGGTTCCTCTCATTGCAGGCTGAAGGTACCGCCTCGGCGCTCTGGCCGCCGTGTGCCCGCGCGCGGGG |
| | | CGCCCCGCTCCCAGAGCCGGGGCCGCGGGAGGGGGCGCAGGCAGCCGGGCGGCAGCGGGCGGCCCCGCCTCTCCGCACTCGG |
| | | GAGGCTGCAGGACCCGGGGTTCCCGCGCGCCCGGGGCCGGGAGACGGGCTGGGGCGCCGGTCCCACCCCTGCGCGTCCTGCC |
| | | TCCTGCGGGCAGCAGCTCGGAGCCTGCGGGAGGGAGCAGGCTGGGCGCGTGGTGGGGGGCGCGCGATGCGGAGGGGGCGGCG |
| | | GCGCAGCCAATCCGAGAGGCGGCCGGCGCCCCCTCATACCGCCCCGCGGCCTCGCCGCCTCCTCCCGCTTTCCTCCTCCAGC |
| | | TCCCACCCGGACCTCTAGAACGGCCGCGCAGAGCGGGGAGGGGGCAGGGGTCCACACCAGAGGCCCAGACTCTGGTCCTTGA |
| | | GTCAAGATGCCCAACCCACTCGCCTACCAATTACGTCCCTTCGTCCAGCTCCAAGCCGGGCGGTAATGGGGTCCAGGAAGGC |
| | | TAAAGGGGACACGCCCAGCCACCGAGGGAGGGGTCTCACCCTTGGAGACCCACTTCGCGGCGGCCGCACAGGTCACGTCTCT |
| | | CTCCCCACCCAGGCCAAGGAATCCCCGGACCTCTTAGAGCTTTGCTTTGGGGCAAGGGCCAAGGAGGCTTTGCCTGCGCCAA |
| | | GTGCAGTTGACTG |
| 406. | ANGPT1 | CCACACACACACTATGCTAGGTCAAGTCCGAAGAGATCTGAGGCCGGGTTTGGGGCGCACGTGG |
| | | ATGGGGAAGATAAAACAAATGAGCTACAGGGAAGATGGTTAGGAGGACAGAGAGAAGAACAACGACGACAAAAAAAAAATGC |
| | | ATGTCAGATAAGAAATACGTCCTAAAGAGACAAGTTTAGATGAGGAGGTGGGGCGGCGAACATGAGTGCGGACCGCACGACT |
| | | TAGCCCTGAGCGCCCGATTGGCGCCCGGCTCGCCGAGCTCCCAGCGCGTCTCGCTCCGGCTCCCCGCGCCTCCAGGGTACAG |
| | | AAAACAGAGTGTTTTGTAGAGCTCCAGGGTCCTAAAGGTGGGGACTGGGGACCCGCAAAAGCTGGGGTGGAGAGCACAGCCC |
| | | TGACCATCTGAGCCCCCGGAGCCAGGGCGTGAGTGAGCGCCTCCACACGCCACCCCTGAAGCCCACCACGCACCTTTGGCAG |
| | | AGAGGGACCCACCTCGCTTACTGCGTCTCCATCGGTTGCCTTGGCAGTGGCTGTAATCCATGCAGTTCAGAATGATGAGGGC |
| | | AAAGGAGAAAAGGCGAAACTGCATCTGGGCGGTCGGGCGGGGGAGAGACGCCTCTCAAAGTCTAGGAACTGGAGGGTTCGCC |
| | | CAAAGAGCCGGCGCCGGCCGCGCTGCTGGGGAGGACTCAGAGGGAGACTCGCCACTCACCCCCGGGCCGCACCGGTCAGTTC |
| | | AGCGCGATCAGCATCTCTCCGCCACGAACCTGAGAGACAAGAAGCGAAAACAGGGTGTGTGGGGGATGGAGAGAGCCCCGGG |
| | | GAGGCAGCTGCGCCTTCGCACGTCCCAATTTAAAGAAGAGAGGGAAGGAGGAAGCGAACCCACTGCCTAGCACGAGCCGCGG |
| | | AGCGGCTTAATCCAGAGCGGGCAGCTGCGGACGAGTTGGAGAGGAAGGTGATTATAATCAGTGAATTAGCAACCTCTGCCGA |
| | | GCCCTAAGCTCCGACCCAGCGGCATAATGCGTTGTGCCGGCGCTCCGGGGAAAGGAAAGAACTCTAAAGGCACTGAGAGCGC |
| | | AGGGCTAAAAGAACGCTCTTAGGCCATTTGGAGTCCACGGCGGTGTACCCAAGCGCCGCAGCTGCGGCTGCCTTGGTGTGGG |
| | | TTGCCTACCGTGCGCACGTCTCGGGCGGCGCGGCCTCCCTAGGCGCGGTGCTCCATCCCGGGCTCGGGAAGCGAGCCGCTTG |
| | | GTTAGCACGTGGGCTGGGGAAAAGTTTGCCGAGACCGGCTGGGAGCGCCTGGCAGCTCTGGAATTCCAGCGGCCGCCGCGGG |
| | | GTTGGCGACGCCAGGCAGGAGCGCGGAGCGGCGGGTGCAGCCACTGGGATGCTCCGTCCCCGCCCGCGAGCGCGGTCCCCAG |
| | | GCAAGGGGTGCGCCCGCTCACACTCTCTGCCTCCCTAACCAATTGTGTCGCTTCCTGCGCTTTCTCCACGGTCACTTCACAG |
| | | CTAAGATTTCTTTCTTTCCGAGCTGTAGAAGGCAGAACGCTCTCGGGAGGACGAAGTGATCCGAAGGGATGTGGCAAGCGCA |
| | | CTTTCCGATGGAGATGCAGACCGGCTGAGGCTTGGGATGCTTCTATAAGTAGGTGGTGCTGTGGTCCAAGGCATCTAAGTCT |
| | | AGGCTGCCTCGGAGTTCCTGGGTCCTAAAGCCAGAAACGTCCCGGTTTCCCTGAGGTCCTAAAGAACGTTGACAGCCAACAG |
| | | CGCGCCTAACTTGGAGCGAATCCTCTTCGGGCTTTCCAGAGTGCGGGGGATAGATAAAG |
| 407. | APOB | CGGGTGCTAGGGCCCGACAGGGGGACCACCGGCACAGGTTTCACCTTTCAGGAGGGAGGCAGGC |
| | | TCCGGAGACCCCCTCCTCAGCCCCTCCATCCCGCGCCCCCCATCCTGAGCCTGCAGGGGCCGCCAGCTGGTCCAATCCCCCC |
| | | ACTCGCCCTGGACCCTGTGGCTGCCCTCCCTCTGGCCTAGGCCCAGGCTGCCCCGGCCAACCTCGTGCCGCCGGCTCCCTCC |
| | | CGCTCCCTCTGCGCCCGCAGAGCGGCCGCGCACTCACCGGCCCTGGCGCCCGCCAGCAGCAGCAGCAGCAGCGCAGGCAGCG |
| | | CCAGCAGCGCCAGCAGCGCGGGCCTCGGCGGGTCCATCGCCAGCTGCGGTGGGGCGGCTCCTGGGCTGCGGCCTGGCCTCGG |
| | | CCTCGCGGCCCTGGCTGGCTGGGCGGGCTCCTCAGCGGCAGCAACCGAGAAGGGCACTCAGCCCCGCAGGTCCCGGTGGGAA |
| | | TGCGCGGCCGGCGCCCGCACCCCATTTATAGGAAGCCCAGGCTGCAAGAGCGCCAGGATTGCAAAAGGTCCAAAGGGCGCCT |
| | | CCCGGGCCTGACCTGTTTGCTTTTCTACACTGGCTTCTCTTTGAGCCTTGAAGAGCCTCGGGGAGGGGGCCCACCTGGGATG |
| | | CAGCCGCAGCCACCAGGGGCTGGGTCCCAGGTGGGTTCCCTTCCCCAAGCGTC |
| 408. | APOC1 | GGCTCAAGCGATCCTCCCGCCTCGGCCTCCCAAAGTGCTGGGATTAGAGGCATGAGCCACCTTG |
| | | CCCGGCCTCCTAGCTCCTTCTTCGTCTCTGCCTCTGCCCTCTGCATCTGCTCTCTGCATCTGTCTCTGTCTCCTTCTCTCGG |
| | | CCTCTGCCCCGTTCCTTCTCTCCCTCTTGGGTCTCTCTGGCTCATCCCCATCTCGCCCGCCCCATCCCAGCCCTTCTCCCCG |
| | | CCTCCCACTGTGCGACACCCTCCCGCCCTCTCGGCCGCAGGGCGCTGATGGACGAGACCATGAAGGAGTTGAAGGCCTACAA |
| | | ATCGGAACTGGAGGAACAACTGACCCCGGTGGCGGAGGAGACGCGGGCACGGCTGTCCAAGGAGCTGCAGGCGGCGCAGGCC |
| | | CGGCTGGGCGCGGACATGGAGGACGTGTGCGGCCGCCTGGTGCAGTACCGCGGCGAGGTGCAGGCCATGCTCGGCCAGAGCA |
| | | CCGAGGAGCTGCGGGTGCGCCTCGCCTCCCACCTGCGCAAGCTGCGTAAGCGGCTCCTCCGCGATGCCGATGACCTGCAGAA |
| | | GCGCCTGGCAGTGTACCAGGCCGGGGCCCGCGAGGGCGCCGAGCGCGGCCTCAGCGCCATCCGCGAGCGCCTGGGGCCCCTG |
| | | GTGGAACAGGGCCGCGTGCGGGCCGCCACTGTGGGCTCCCTGGCCGGCCAGCCGCTACAGGAGCGGGCCCAGGCCTGGGGCG |
| | | AGCGGCTGCGCGCGCGGATGGAGGAGATGGGCAGCCGGACCCGCGACCGCCTGGACGAGGTGAAGGAGCAGGTGGCGGAGGT |
| | | GCGCGCCAAGCTGGAGGAGCAGGCCCAGCAGATACGCCTGCAGGCCGAGGCCTTCCAGGCCCGCCTCAAGAGCTGGTTCGAG |
| | | CCCCTGGTGGAAGACATGCAGCGCCAGTGGGCCGGGCTGGTGGAGAAGGTGCAGGCTGCCGTGGGCACCAGCGCCGCCCCTG |
| | | TGCCCAGCGACAATCACTGAACGCCGAAGCCTGCAGCCATGCGACCCCACGCCACCCCGTGCCTCCTGCCTCCGCGCAGCCT |
| | | GCAGCGGGAGACCCTGTCCCCGCCCCAGCCGTCCTCCTGGGGTGGACCCTAGTTTAATAAAGATTCACCAAGTTTCACGCAT |
| | | CTGCTGGCCTCCCCCTGTGATTTCCTCTAAGCCCCAGCCTCAGTTTCTCTTTCTGCCCACATACTGGCCACACAATTCTCAG |
| | | CCCCCTCCTCTCCATCTGTGTCTGTGTGTATCTTTCTCTCTGCCCTTTTTTTTTTTTTTAGACGGAG |
| 409. | AQP1 | CATCCAGAGGAGGTCTGTGTGGTGTGGGGCGGGCCAGGAGCGAAGAGAGGCCTTCCTCCCTTTG |
| | | TGCTCCCCCCGCCCCCCGGCCCTATAAATAGGCCCAGCCCAGGCTGTGGCTCAGCTCTCAGAGGGAATTGAGCACCCGGCAG |
| | | CGGTCTCAGGCCAAGCCCCCTGCCAGCATGGCCAGCGAGTTCAAGAAGAAGCTCTTCTGGAGGGCAGTGGTGGCCGAGTTCC |
| | | TGGCCACGACCCTCTTTGTCTTCATCAGCATCGGTTCTGCCCTGGGCTTCAAATACCCGGTGGGGAACAACCAGACGGCGGT |
| | | CCAGGACAACGTGAAGGTGTCGCTGGCCTTCGGGCTGAGCATCGCCACGCTGGCGCAGAGTGTGGGCCACATCAGCGGCGCC |
| | | CACCTCAACCCGGCTGTCACACTGGGGCTGCTGCTCA |
| 410. | ARHGAP22 | CGTTTGCCAAGAGGCACATGCGGTGCCCAGAGAAACCCCAGAAAGTTGGACTTACCCCTCCTGG |
| | | CCTGCCTGATCTTTGGGCTCAGCATGTTCTTGCAGCCGTCCGGCCAGCCCCGCAGGGCCGTTCATGCTGTCATCCACTTGCT |
| | | TTTGCTCGTCCTCGCGCCTAGTCGCCCCTCATGTCCTGCTCGTTCGGGGCCCCGTGGCCGCTGGCGTCACCCGTCAGGCTCC |
| | | CTCGGCTACCTCTCCTGGCTCCGGACGGACGGCTCGCCTTGGACTACATAAACCTCGATGCATTATTTATCCATCCCAGAAT |
| | | TAATTCCCATCCAAGCGGACCATTAAAGCCTCAGTAATCACTGCTGATCAATCACTGGACCAGGGCGGGGCAGTCCCTCCGC |
| | | CTGGCCTGGGCGCACGCGTGGCTGTCCAAGCTGAGCGCGGGGGCGGGGCGGGGCCGAGAGGGGCGGGGCCAGCCAGAGACGC |
| | | GGGGCGCGGTCCTGGCCCGGGAGAGGGTACTGGGGTTCCGGCCATCCTTTGCTGAGGCTGGAAGAAATGCGCCTTTCAGCCG |
| | | CCGCCTGGGAGCCGCACTCCCTGCCAAGCCCGGGTGGGATAGCGCTTTGGTGAGAAGGTAGCCGAGACCCCCCCCCCACCCC |
| | | AGAGCCCAATCCACGGGGACCCGCTTATGAGCTCACTGTTTTACGTGTTGAAAGTCAGGTGT |
| 411. | ATP8B4 | GTCACAACGCGAGGAGGTCCAGACTTGACCCCAGACCTCCAGACTCGAGCTGGGAAGTGGCCGA |
| | | GGCTCCTGGTGAGCCGCAGGGCAGCCTGCCCAGCCCCGGGAGGACCGCGTGGGAGGGCCGGGAGAGCAGGTGGCTGCCCCCC |
| | | ACAAGGGGGCGGGCTCAGCCGGCCAGTCCTGCCCGGAACCCCCGGCAACGCGCATACGACTACACCTGCTCCGGAGCCCGCG |
| | | GCGGTACCTGCAGCGGAGGAGCTCTGTCTTCCCCTTCATCTCACGCGAGCCCGGCGTCCCGCCGCGTGCGCCCCGGCGCAGC |
| | | CCGCCAGTCCGCCCGGAGCCCGCCCAGTCGCCGCGCTGCACGCCCGGGGTGAACCCTCTGCCCTCGCTGGGACAGAGGGCCC |
| | | CGCAGCCGTCATGCTTTCCGCCATCTACACAGTCCTGGCGGGACTGCTGTTCCTGCCGCTCCTGGTGAACCTCTGCTGCCCA |
| | | TACTTCTTCCAGGACATAGGCTACTTCTTGAAGGTGGCCGCCGTGGGCCGGAGGGTGCGCAGCTACGGGAAGCGGCGGCCGG |
| | | CGCGCACCATCCTGCGGGCGTTCCTGGAGAAAGCGCGCCAGACGCCACACAAGCCTTTTCTGCTCTTCCGCGACGAGACTCT |
| | | CACCTACGCGCAGGTGGACCGGCGCAGCAATCAAGTGGCCCGGGCGCTGCACGACCACCTCGGCCTGCGCCAGGGAGACTGC |
| | | GTGGCGCTCCTTATGGGTAACGAGCCGGCCTACGTGTGGCTGTGGCTGGGGCTGGTGAAGCTGGGCTGTGCCATGGCGTGCC |
| | | TCAATTACAACATCCGCGCGAAGTCCCTGCTGCACTGCTTCCAGTGCTGCGGGGCGAAGGTGCTGCTGGTGTCGCCAGGTGA |
| | | GCCCCGAGGATCGCCCTGCCCTGGCACCAGGGCTTCTCGGCGCCTTGACTGACGAGCCACAGCGTGGCATAAGGGGTTCGCA |
| | | GAGGGAGGTTCAGATCGGAACTGTAGGTATAGAAAAAGGTTGGCAGTGTTTCCTTGAATCGCAAATAATGATCTTTTAGGAC |
| | | CACCTGTTGTGGAAGC |
| 412. | AZGP1 | GGGGCTACAGGTGTGCCCCACCACACCCGGCTCATTTTATTAATTCTTGGTAGAGACCAGGTCT |
| | | CTCTCTGTTGCCCAGGCCAGTCTCGAACTCCTGGCCTCAAGCGATCCTCCCACCTCGGCCTCCCAAAGGACTGGGATTACAG |
| | | GTACGAGTCACTGCGCCCAGGCTTTTCTCCCTTTTGACTCATTTTGAGGCCCCCGTCGTAGGGTACACGCCCTGGGAATCGG |
| | | GGGTCTTCCAGTATAGGGAACCACCGAGATGCGGGCCCTCCGAAGTCCTAGAGAGGGCGCCTCGGCCGCCGGCACCCGCCAC |
| | | ACTTCCGGCCTTTGTGGGCCGCAGCGACGGCGGTCTGCGGCTGTCGGTTCTGTTTGTTGCTGTCACTGCTGTTTGTTCTTGC |
| | | CAGCGGCTAGGTGTGTAGTTGCTTGGGGCTCCTAGCACGAGGCCTCTGTCCCCAGGCACGCAGCGCCAGTCTCCGGCTGTTG |
| | | CGCCCTGGGCGCCGCCTCTCTCCGGCCCCCCTTTGTTTCCTGAGCCAGCTGGGAAGACTAAAAGGGTGGGTCCTGGGAGCGG |
| | | CCCATGGTTCGACCCTTCTTTTCAGGCATCCTTGGAGCATGCAGGCTGCAGATTTCAGGCCGACCCCGCTAGCACCAAAGTC |
| | | CATGCTGCTGTCCGTGGCAGCTCAGTTTGAAAATGGCCTGGTAGAAAGTCCAGGTTGTTG |
| 413. | BAALC | GAAGGAAGGAAGGATGGATGGATGGATGAATGAATGAGCGGGTGAGCGGATGGATGGATGGATG |
| | | GATGGATGGATGGATAGATGGGCCAGAGACTGGGAACGGTAATGCCGGCCAGACGGGTCCGAGCGCCTCGGGAGCCCCAGCC |
| | | CTCCTCCGGCTGCGGGGCCGCTGCTCCTGCTCCCGGGCCGTCACGGAGCCCTGAGGGAGGGGGCGGGCGAGCGGGGGCCTTG |
| | | GGGCGGCCAGGGCGCTGGGAGGGAGGACTGGGCGGTGGGGACCGGAGGGGAGGGAGGGCCTTGGAGGCGGAGAGGAGGGACG |
| | | GGCTGGGAGAGGGCCCGGACTAGGGGCGGCGGGCACCGCAGGAGCTCCGCGCGGCTGCAGCGCGGGCGGGAGCGGGGACGCG |
| | | ATGTCGCCGCCGCCGCCTCCTTGCGGGCCGGGGCTGCGCCTCCGGGGCTGAGCCGCCGCCAGAGCCGACAGCCGAGCAGCCG |
| | | CTGGGCGCTCCCGCGGCGCAGGAGGATGGGCTGCGGCGGGAGCCGGGCGGATGCCATCGAGCCCCGCTACTACGAGAGCTGG |
| | | ACCCGGGAGACAGAATCCACCTGGCTCACCTACACCGACTCGGACGCGCCGCCCAGCGCCGCCGCCCCGGACAGCGGCCCCG |
| | | AAGCGGGCGGCCTGCACTCGGGTAAGTGGCCGGGCCCCTGCAGACCCTCGCCCGCCCGCTACCCCGGGCGCCTTGGCCCGGG |
| | | CACTGAGAGAGGAGCCGGTTCCCTGAGGAATTGATGGCGCGGCGGGGGTGGCTGGGAGGAAGCAGAGGCAGGACCTGCCCAC |
| | | TGATCAGTGGACAGATGCAAGCCCAGGGAGTGGGGTCGACCAGAGCCCCTTACCGAAGGCTG |
| 414. | BAI2 | CTGGTCTGTACGTGAAGGCTCAAAGGACTCTTGACTTGCTGCTGGGGTGAGCCCTCCACCCCAC |
| | | CCCAACCCATCCCACCCCCAGAACATATGGCCTGACAACCAGGAGTGTTTGGGCATGGCTAGCACGAGGAGGCACCTGGCCT |
| | | GGCCCACCAATCTCCTCTCCCCAGGGATTTCCCAGCGTCAGTCCTGCAGCCACCGCTGATGCTCTGGCTGCGGAAACCCGGG |
| | | GTTGCCCGCGGGAGAAGGGGTGGCGGGTCCCAGGCTTCGGGCAGGGGGCGCCAGAGGACGCCCAGGAGCCCGGCCGGCAGCT |
| | | GGCGAGGCGGGCAGGCAGGCGGCATCTCTCGGCCCGGGCGCCCCTCCCCCCGGCGGGCAACAGACCCGTCGCCTGCTCTGCA |
| | | CGGGCCGCGGCGCGGAGGACTTCCCCCGGCTGTGCCCTCGGACCAAGCTGACTGCGGGAGTGGCCGCCAACTTCACAGCCTA |
| | | CAACCCCCTCCGGATCATGCCGGCCCTGGGGCTGCGGGAGGGTGCGTCTGGTGCCCAGCTCTATGACCAGGGGTGGGAGGAT |
| | | AGGGATGGGGGATGGACAGATAGACTCCGGCCAAAGATGGACACGGGAGTCAGGGATGCGGGTCCAGTCGGGATCTCAGCTG |
| | | GAGGGGAGGGGGCGCGGAGCAGACACGGGGGACACGGCCCCAATTCCAAGCTGAGGGGAGGGGACTTGCAGCAAGATTGGCG |
| | | AGGGAAGGCGGGACTCCGGTCAGATAGATATGGGGGAGGGGGCGCTAGGGGCAGAAATAGAGAAGAAGCATGGTCGGGGTAC |
| | | AGGCGCGGGGGAGGGTAGAAACTATGGGCAGACATGGATGGGGACAGGAAAGACTCCGGGCAGTGATGAAGTGGAGGAGAGG |
| | | AGCAGGGTCCGGCATTCAGACCGTGACGGGAAATAGAGGGGGAGACACAGTTGAGCTGCAGGTTAATGGGGGGGCTCTAATA |
| | | AAGGTGGGGGGCACACTGCCCGGATCCAGGCCGAGATAGGGAAAGACTCGAGACAGCAAGCACGGTCTGTGTCAGAATTCTG |
| | | GAGGGGGAGACTTTAGCATAGATGGGTAAGAGGGTCGGTCCAGGCTGGGGGAGGGGGGGGGCCGAGATTAGAGATTCACCAG |
| | | CGATTAAGGGGGCCTCGGCCCTAGGTTTGGGAGTTCCCCAGAAGATGGGGGTGGGCGAACGCTGAACGAACTCAGTTCGGGC |
| | | TGGGGGAGAATTCAGCAGAGACGGGTTAGGAGAGCGCCCCGAGTCCGGGCCGGTGCGGAAACTCGGGACCGCGCCGGCAGGG |
| | | CTCGGTGCGAGGCCAGGTCGGGGTCTGGGGCCGAGCACTCACCTGGGCGCCGTCAGCAGCAGGAGCGGGGGCCGGCGCTGGC |
| | | GGGGGCGGCCACGGGGCGCAAGTTTGCCATCCCTAAGTCGGGGACCGGGCCGGGCGCAGGGTAGGTAGCTGCAGCCGCGCGG |
| | | AGGGCCGAGGCTCCCGCTCTCCCGGGCGGCGGGTGCAGAAAAGGCGCCGCGGAGCAGCGCGGGGCGGGCGGGCGGGCGGCGC |
| | | CGGGCCGGGCGCGGGCTCCTGCCGCCGCCGCCGCCGCCGCCTCCTTGCCGCGCCGCCCCCCGCTCCCCCGCTCCCCCGCCCC |
| | | GAGCACCGCCCGCGCCGCGCGCCGCCTCCTATTTGCGGGCGGCGGCCGCAGCCCCGGGCTCTGAGAGCCGGTGCCGCATCCT |
| | | CTTCGGTCTCTGAGCGCCGCCGCCGCTGCCGCCGAACCCGGTTCCTCCGGCCGCTCCGGCCGCTGCCCGCAGCGCGCGCCGG |
| | | GCCGAGTGACGGCCGAGGCGGGACGCGGCGCCTGCGCGGGCCGGGCCCGGGAGGGGGCGCGCGCCGGGCCGGGCGCGAGGAG |
| | | CCGCGGGAGAAGGGGCGGGGGCGACGCGCGCCGCCTCCTGTTAAAGGGGGAACAGCGCCAGCCGAGCCGATCGTGCCCCCCA |
| | | CCTCCCCGCCTTGGGCCCCAGGCGCCGGCGCGGGAGAAGCCCTGGGTAAGCCCCCGCCCAGCCCGCTGCTTCTTGGACCGCA |
| | | GAATGAGGCGATCCGCGACGCCCCTCCAGCTATACCCATGCCGGAGGCTTGTCGCGCCGCCAGGGTGTCCCGGGCCCTGCAG |
| | | GCTGAAGCATTTGCTCACCTTCCCATGTGCATTGCATATTGCATGTCCCTGTGCATGCAGGTGCCAGTCATGTGCGCTGAAC |
| | | TTTGCATATGCATACAGGTGCCCTAAGTGTGCACTGCCATGTTGCCTGGGTAGGTGCATGCAAGTGCCACACACATGCACGG |
| | | TGTATTAACATGTGCCCAAGTCCTGA |
| 415. | BCL11A | AAGGGGAGGAGGGAAGGGGAAGCTCACACCAATGGACACACATCAGGGGCTGGACATGAAAAAG |
| | | AGACCAGGACAAGCCAATGGCCAGTGCGGGGAGGGGGAGGTGCGGGGCGGGGGGCTCCGCGGACGCCAGACGCGGCCCCCGG |
| | | GGGAGGGGCGGGCCGAGGGGAGGGGGCGCTGGGGCCGCGGGCTCACCAGTGGCCGCAGCGAGCGCCGCGGCGGTGGCGTGGC |
| | | CGGGAGAGAAGAAAGGGGTGGCAGGGGTGGGAGGAAAGGGTGGGGGGGAGCAAGACGTGCGCCCTGCTCCCCCCCACACACG |
| | | CGGACTCTAAAATGAAAGATTATTCAAAAAAGAAAAAAATAGAGCGAGAGTGCACCGGGAGGCTGCAGCCCCGGGCTGGGGA |
| | | AGCGCGGGCGGAGGGAAGCCAGGTAGAGTTGCTCCCGGACACCCTCCTCCGTACGCACACCCACTTCCCCTCCCCGCCGCCG |
| | | CCGCGCTCGCTCCCCGCGTGTGGACGCCAGGGGCCGAAGTAAAAGCCCCGAGCCCGCGGCTGCGCTCGGGAAACTTTGCCCG |
| | | AGGAGAGGACAGCAAAGAAAAATCACCCGAAGTTGAGAGCTGAGCCTCCAAGTTACAGCTCCGCAGCGGGCGAGGGGAGGTG |
| | | GGAGGGAGCGCACGGCAACGCGGAATCCAGCCTAAGTTTGGAGGGCTGCGGGTCCGGAAGGGCAGCGCCCAAGTCTCCAGGA |
| | | GCCCGCGCGGCCTGGAAAGAGGGGACCGGGGAGAGGCAGGCGGCGCAGGCCGGGGCCCGAGGGCGCCCCCAAGGCCGAGCCA |
| | | GGGACCGGGAATCTGAGCGCCCCGCCAAGCGACTGGGTCTGAATGCAACTGAAAGCGCCGAGTCCCCGGCTCCTAAGGGTCG |
| | | CAGAAAGAGAGCCCGAGGAATAAAGAACGAGGAGCCAGAGTCTGGCCCCCGGAGGGGAGCGCCGGGGGCCTGGGCAAGAGAG |
| | | GCACCGAGGCGTCCACCTCTCTGAGAGGCCGAGGACAAAGCGCGGGCAGTCCCGGGAGTCCCAGTCCCTGGGAATGTGCTCA |
| | | CGGCGCCGCGGGAGGTCCCCGAGCCGGGTCCCTGGGAGGGACACACCCCCTCCCACTCCAGAGCAACTGCAAAAGCACAGAG |
| | | GGATGGGAAGAAAATGTTTCTTACGGGCAGAGCACAACTGTTCCTGTGCTGTTACCCAGGGAAAGAGAAAATGGGAATGGAG |
| | | AGAGAGCGACAGGCTCGCAGGAGCAGCGGGGAGACCACGGTGGTGAGATGACCGCCTCGGGTCACCGCCGCGCTCCCCAGGC |
| | | GCCGCCGGCCAAGGCGCACACCCAGGAGCCAGCAGCCCCCTCTCGCCTTTCTGCAGACGTTCCCTGGAAATTAGGGAAGACC |
| | | TGTTGGAAATAAAACCCTGTCTCTTTAATGCACACACGCACACACACGCGCGTGCTGCCGCTGTCAAAAAGCGGGCTTCACT |
| | | CTGGCGTGGGATACTTTCAGTTTTACGTAAAATGTACAGCAACAGCACCCCCTGCGATTGGAAAGTTTATTCAGAAACGTGC |
| | | CTGTAAAGTCCCCTGGCGCCTCCCGCCTCCCAGCCTCCCTGCCTGCCAGCACCACTCTGCAGCCGCCGCCGCCGCCGCCGGG |
| | | GAGATCGCAGTCCGATTCCAAATCTGGAAGGAGTTGGGGGGA |
| 416. | C10orf38 | TCTGCCAGGGGCTTTTCTTGTCTTCTCCTTGGTCATCATCATCATCGTCTTCCTCTTCCTCGTG |
| | | GGCAGATCTTCTCTGGTGGGGGCTGGCTGCTGGCTCCGAGGGGGCATCCGCAGTCCGTCTGGTCGTCTCCTCCTGCAGGCTG |
| | | GGCAGCTGGCCACCACTTCTCCGACTCGACCCCTCCAACAAGCATCGCAGGGCACTGTCCTCGGGGGTACAGACCGTGGTCC |
| | | CACATTCGCTACCACTCTGTTCCACGTCATCCAGGTACACGAGCTGCGTGTAGGCCGTGCTGTCTGGGGCTCGAGGCTCTTT |
| | | CTGCTGGTGCTCTTGGACGGGCGGGTAGTTCTGCTGCAGAGACAAAGCATCTCCCCTTCCCTTCCGGGCTGATTTTGGTTCA |
| | | TTCATATCTACGCCAGAGTCCAAACTGGCATCATTACTTCC |
| 417. | CD3D | TAGGACTGGTGGGAAAATAAGAGAGCAGACCCCCAACAGAGGTTGCGAGCAGAGCCAGGGATCA |
| | | CAGAGTTTTATCTGCCCTCGGTACGCTGATTTCCAAAACCCAGCCTCATATTCTATACTCCAAAGCGCACTGCCAGGTGGGC |
| | | CAACTCCAGCCCCCACAATCCGATGCCAAGGCCACTTCTTGCCACTTCCTGCCCGCCTCCAGCCCGCCCCTAACAAGCTAGC |
| | | GCATGCGCTGTAGGACTATTTGCGCGCCGTTCCCCCCGGTGAGCCTGCACACCGTGCTTCCGGCGCGGAGATGAACTTCCAC |
| | | ATGCCCCGGCGCTTGTTGATGACGTAACTTCCGGTTGCTGTGCTGAGTCGGAAGTGGGAACCCTTCGGCCGCTGAGATTCTG |
| | | TCGTGTCGTCGCTGCTGGCACTTCAGGCTCTGGTAAGACAGGCAATAGCTTCAGGGGACGGGGTCGTTGACTTTGCGTTGTG |
| | | ATATTTTCTGCTGGTCCAATGCCACTTCAAGCCTGTTCACTCTTATCTTCTCCAAATTCCTTCTCCTATGCTTTGCTAACCA |
| | | GGGACCTTCTCCTTCTTGAACTTCGACCCTGTCTGATTTGGAACCCC |
| 418. | CDC42EP4 | TCCATTCTCCCAGGGAGAAAAAGAAAACTTCCAAAGCCGAGGCCGCAGTCACTGGGAAAACACA |
| | | GGGAACCTGGTTTGAGTTAGCAGCGAGCACCCCTCCCCCAGTCACACACACACACACACACACACACACACACACACACACA |
| | | CACACACACACGCACTTCAGTCCTCTCCCAGGAAGCTGGCTCAGCCTCAGCCCCGGCTCCCGCCCAGCTCTCCAGCCCTCCC |
| | | GGCCGGGGCACTCACCGGCTGACGGGATCCCCGCGGGCAGGATGGCTCCTGGGGGACGCGCCGGGGGCAGGGGGCAGTTTTG |
| | | TAACCCCGGCTGGGGGCGCTCCGGAGAGGGGGCGGGGCCAGGGACGGCCGATCCGCGCGCACCCGCCCCAAGCGGGCGATTA |
| | | AATCATTAACCGACCGCTCCCGCCTGCGATCCCGCTGAGCGCGCTGAGCTCGGGGCGCACTAGCCCAGGAGCGGCCGTCTCC |
| | | CCGGCTCCAAGGGGGCGGGTGGCTCGACTGCCCTCCCCCCGCACCCCGCCAGGAGGCGCCTCGGCCTCCCGGGCTTCCGAGG |
| | | GAGCGGGCGGGGCGGGAGCCTGGCGGGGGAGGGAAGGGTTCGCGGGGCGGGAGTAGGGGAAAGGAGGACCATTTGTTCCTTC |
| | | GGCCATTCCCTTCCAGACAGCTCCTTCCCTCTGGCTTCCCGGCCGGGTGACATTGGGTGGCGTGTGCTGCAGTGTGTGTACG |
| | | AGAGCGAGATGTGTACACGCCTCTGCCCGTACAGGCGGAGAATTCGGTTCTTATTCATGGAGAACTCCGGGCGCACACACGC |
| | | CCCCCTCGCGGAGATCGCGGGCCGCGCGCCGCCGATTGCTGCGCTCCTGCCCACACCGACACACACCCCATTCCTGGGAAGG |
| | | GGGAGGCCCTTTAAAACCGGCCCCCAACCCCCTCAGCGGCACCCTTGGCCAGGGCCGGGAGTTCCAGCGTGGAGAGAGGAAA |
| | | TGGAGTCCCGGCAACGCTGCGCCCGGCTCCCTGCCACCTCCTCCTCCACACATCCCGCTCTGCCAGGACACTAGGTGCGCAG |
| | | CTTAGGGTCCCAGGCACCGCGGTGGGCGCGGTGAAAAGCGCGAGCGCCGAGAAGTGGGGGCTCCGCGCCCTTCTCCCCGCGC |
| | | CGCGGCCTCGCATCCCCAGCTTCGATCCTCTCTGCCCCACAGGGGGACTCGAGACCCCCAAGCCCACCCAGGGCACCCAGCG |
| | | CGAAGGCGGCTACCACAGACAGAGGCGGTTGCCCGTCGCCTACCTCGGTCCCCGGAGACCCGAGCTCTGGGCCGGCTGCGCT |
| | | GTCCCCTTCGTGGGCTCCGCGCGGCGGAGGGGCTGGCGGCCCCTGTCAGCGCCGGCGTTTGTTTCCCTCTGGCTCTCGGGCT |
| | | TTCAGGGCTGGGAGCCTCCAGGACACTCCCCTTCCAGGCTCCGCCCGCGCCCGCCCCGCGCAACCCTCCCCGCGCACCCCTC |
| | | TGCGCTGATGTCACGGCGCCGCAGCAGCCAATGGCCGTCGGGAACCTCTGCCCGACTGCTCGGCCTCCTCGGTTCAAACTGA |
| | | AAGCCTGAGACTTTCCCAACTCCGTATGGGGCAGCCTGGGCCCGCCCGGCTTCCGGGAGACACTCCCGCCCCAGCCTGCGCT |
| | | CCGGCCGCGAACCCCCACTGGGGCCCCCAGGGCCAGGGGCGCGTCTCCCTCCGGGGGCCTGGCGCGAGGCTCTTTTGAGGCT |
| | | CTTCCCCATTCGAGCCGGGGCAAGAAACCAAGAACTCCAGCCCGGGTGGCGTTCCTCGTTCCTGCTCCCTCCCGGACCTGCT |
| | | AACCACCTGGCTGCAGTACTGTCGGGAGATGGGGCAGGAGGGCTGGCGCCACCAGCTTCGTGGGTGCCATCCCAGACTCTCG |
| | | CTGTAAACGAGGGAGCGGGGAGGAGCCGGCCAGCCCCGAGGCCAGGCTTGGACTGGGCGTCAGAAATGTTTCTACAGGGTGA |
| | | CAGCCCTTGAGCTCCCCGCATCCCCGTCTGTGCCTTTGCCTCCTAAGTATCAGCTGAGGCTAAATTTGCACCAGGTGAACGT |
| | | CAAGGAGGATCAACTCCTGAGTCCCAGAGCCTGAATCCAAGTCCCAGGCCTTGGACTCTATTCTTTTTAGAGGA |
| 419. | CDH5 | CCCTGAGGCAGAGGGTGAGGAGTAGGTACAAGGAAGTGTAGGGAAATTTATCTTAAATGCGCTT |
| | | GTTTACTCATGTTGTCCTAAAACCGACCTTTCATCCGAGTGCATGACTGCTCCCTGAAAGGGGGAACAATAATGTTAATTAC |
| | | CCGCAGATTGTGTTTGCCCCAGGCTTTTGGCATTATATCTAGCTGCTCTTATGTGAAATACCTGTGTCTAAGTACTCCTTTC |
| | | ATCGTCACTGGCCAGAGTCTGAATGTGTCTCCTCTTTCCCCAGATCTGTTCCTCCTGGGAAGATGCAGAGGCTCATGATGCT |
| | | CCTCGCCACATCGGGCGCCTGCCTGGGCCTGCTGGCAGTG |
| 420. | CDKN2A | TCCTCTTCCTTGGCTTCCCAAGCCCCCAGGGCGTCGCCAGGAGGAGGTCTGTGATTACAAACCC |
| | | CTTCTGAAAACTCCCCAGGAAGCCTCCCCTTTTTCCGGAGAATCGAAGCGCTACCTGATTCCAATTCCCCTGCAAACTTCGT |
| | | CCTCCAGAGTCGCCCGCCATCCCCTGCTCCCGCTGCAGACCCTCTACCCACCTGGATCGGCCTCCGACCGTAACTATTCGGT |
| | | GCGTTGGGCAGCGCCCCCGCCTCCAGCAGCGCCCGCACCTCCTCTACCCGACCCCGGGCCGCGGCCGTGGCCAGCCAGTCAG |
| | | CCGAAGGCTCCATGCTGCTCCCCGCCGCCGGCTCCATGCTGCTCCCCGCCGCCCGCTGCCTGCTCTCCCCCTCTCCGCAGCC |
| | | GCCGAGCGCACGCGGTCCGCCCCACCCTCTGGTGACCAGCCAGCCCCTCCTCTTTCTTCCTCCGGTGCTGGCGGAAGAGCCC |
| | | CCTCCGACCCTGTCCCTCAAATCCTCTGGAGGGACCGCGGTATCTTTCCAGGCAAGGGGACGCCGTGAGCGAGTGCTCGGAG |
| | | GAGGTGCTATTAACTCCGAGCACTTAGCGAATGTGGCACCCCTGAAGTCGCCCCAGGTTGGGTCTCCCCCGGGGGCACCAGC |
| | | CGGAAGCAGCCCTCGCCAGAGCCAGCGTTGGCAAGGAAGGAGGACTGGGCTCCTCCCCACCTGCCCCCCACACCGCCCTCCG |
| | | GCCTCCCTGCTCCCAGCCGCGCTCCCCCGCCTGCCAGCAAAGGCGTGTTTGAGTGCGTTCACTCTGTTAAAAAGAAATCCGC |
| | | CCCCGCCCCGTTTCCTTCCTCCGCGATACAACCTTCCTAACTGCCAAATTGAATCGGGGTGTTTGGTGTCATAGGGAAAGTA |
| | | TGGCTTCTTCTTTTAATCATAAGAAAAAGCAAAACTATTCTTT |
| 421. | CDKN2A | CGAATGGGGAAGCCTCCACCGGCGGTTATCTCCTCCTCCTCCTAGCCTGGGCTAGAGACGAATT |
| | | ATCTGTTTACGAAATCACACCAAACAAAACAAGTGCCGAATGCGCCCCGGACTTTTCGAGGGCCTTTCCTACCTGGTCTTCT |
| | | AGGAAGCGGCTGCTGCCCTAGACGCTGGCTCCTCAGTAGCATCAGCACGAGGGCCACAGCGGCGGGCGCCCCTGGCGCTGCC |
| | | CACTCCCCCGTGAGCCGCGGGATGTGAACCACGAAAACCCTCACTCGCGGCGGGCCGCACGCGCGCCGAATCCGGAGGGTCA |
| | | CCAAGAACCTGCGCACCATGTTCTCGCCGCCTCCAGGGCCGAGCTCGGCAGCCGCTGCGCCGCCCTTTGGCACCAGAGGTGA |
| | | GCAGCGCCACTCCTGCCCCCTTAACTGCAGACTGGGACCCACGCACCGCCCCCTGCCCATCTCCGCCCCGCAGGCGCGCACC |
| | | CGCCTTCCCTGAGCGCGCCCGCCCCCCACCTTCACCCCCACCCCCACCCCACCCCCACTCCCACCCGGACCTCCAAGATCTC |
| | | GGAACGGCTCTGAGCCCTGCGCACGCGGGAAGGGCTGCCGGAGGCGCCCGTAGGGAGGCGCGCGCGCGGGCGGCTCAGGGCC |
| | | CGCGTTCCTCTCCCTCCCGCCTACCGCCACTTTCCCGCCCTGTGTGCGCCCCCACCCCCACCACCATCTTCCCACCCTCAGC |
| | | GCGGGCGCCCCGCGGTGACGGCCCAGGGGCCGGACGCCTGGAACGCAACTCCAGGCAGCTCGCCCCCTAGCTACATCCGTCA |
| | | CCTGACACGGCCCTACCAGGAACAGCCGCGCTCCCGCGGATTCTGGTGCTGCTCGCGTCCCCGCTCCCCTATTCCCCTTATT |
| | | TTATTCCTGGCTCCCCTCGTCGAAAGTCTTCCATTCTTCAAACTAGATTATTTAAAAATGAAAAAGGAAGAAAGGAAAGCGA |
| | | GGTCATCTCATTGCTCTATCCGCCAATCAGGAGGCTGAATGTCAGTTTTGAACTAAAAGCCGCTCCGCTCCTCTTCTAGATT |
| | | TGGAAAACAAGCGAAATTAAACTAAACCGCTGCACGCCTCTGACGCGACATCTGGACACGGCGCGGCGCTGGCGCTGCCGGA |
| | | GCTGTCGACCCGGCCTGGCGCCGGACTAGGTAGGTGGAGTCGCACCCGGGGGTCCCAGCTGGGTCCGGGCGCCCATTCCCCT |
| | | CCCAGCTGCCCGCGTCGCCGAGGGCGCCTGGCTGGGACAAGCACCGAGTCCTTTGTGTCTAGCCCATTTTTATTTTCGGTTT |
| | | TAACCTTCACGACAGCCGCGGAGCATCTGAGCGCTTCTTCCTCTTTCCTCTTCCCCCGCGCTCCCCTCCCCTGCTGGCCGCT |
| | | CCCCTCCTGTCGCCGCGTCCTCGCGTAGAATGGTTGTCTTGGCGACCGTTGGCCGCTGCCGCCTCCACGCTCTGCCCCGCGC |
| | | CCAGACACCCCGACTCCCCTTGATCCCGCCGCCTGACTCCTCGGCGTACGTTCTCTCTCCGGTCTCCCCCTCCATGTCCCCT |
| | | CCTCCCCTTTTCTTCCACATCACCGATCCTTTCTGGACTCTCTCCCTTCCTCCTTTCCAGCTGGGAGACAGGAAAAGCGGTC |
| | | CTGTTTGGGAACAGTAAAAGCAGGGCAAGGAAAGGAAGGAGCGGCAGAAAGGAGGGGTGAGTCGAGGACACAGGGGCAGCCG |
| | | GAGAATGCGGAGGAGCCGGGTCCTGAGCGCGGTCTAAGCGAGGCTCGGCTCTCGTCCAGGAACTCGGACGCGGGCTCGCCGG |
| | | CTCTCCGCGCGCGGGAAGTCGAGCCCAGGACGCCGCCTTCAGGCCGGCGCGCTGACCCGGTGCCCCGACCCGGAGCCTGCGG |
| | | TCTGCCTGGATCCGTCCTAAACCTCGCGGGCTGGACCCGCGGCCTGAGTGGGTGGGTGTGTGCCAGAGGATTCGGGACTAGG |
| | | CCCAGCTCCGGGAACCTGGAAATGTGGCCCGCTTCTCAGTGGCTTCCTGTTCATGCGCT |
| 422. | CDX2 | CGCAAACAATGCAGGACAAGGCGATCTTATCAAAGAAAAGCCCTTTCAATGCCTTAATAACAAC |
| | | CACATTCTGTTTGAATTACCACTACTGAGCAAATGGCGGCCCCGGCTTTCATCCCCCTCCCCGCCAGGCGCATTAACATAAA |
| | | CACGACCCTGCAGGCGCATTTGAATGGGGCTGCGGCCCCGACCCCGCGCCTGCTATGAAAGTAAAGGGAACTAACGCGACTT |
| | | TCTCCGCGGTGGACGCTCGGACACGCGTGACCGCCTCAAACCCACTCCAGGCTGCCATTGGTACTCGCCCCTTTTTACAGAT |
| | | GAGGAAATGGAGAATCAGACCGGGTCACGCAGATAGTATCAGGCGGGGTTGGCACCGGAGCCTGGCTGGAGCCACAGAGGAC |
| | | ACCTCCGGAGCTGAGGCGCAGGGCAGTTCCCTCAGCCGAACACCCCGGGAGGGAGACTCGCCTGGAAGTGTCTCGGAGCGAC |
| | | CCTTGCTCGGGTGCTGCCCCTTCAGTGTCCCGGACGTGCCGGGCAACGCGATGACCAGACCCTGAGCGCTCTTCGACCCATG |
| | | CCCCAAGGGCCTTGGAGGCGCCCCACACCCCCTGGGAACCTAGGAGCGGGGCTATGGAGGGGAGGGATGAGAGGGACTTGTC |
| | | AGAATTCATTATTGACTGCGAAAGTCACCACCCTCTTTCCGGGCCCTAAAAGAGACAATGGCAGGGCTGGGAAGGTGTATAT |
| | | CAAAGCAAGCCAGGCCGCGTCCCCCCCTCCACCCCCACACCTTGACAGATGACGCTCCGCAGAGGAGCCCGGCTCCGGCCCG |
| | | CGGGCTGCGGCCACCCCACATTAACATCACAAGGGCACCCGGCGCCGACTGCGGCCTTGCCACCTCGGCGCAGGACTTCACA |
| | | GCGGCCTCTCATCTGCTGACCCCGCGGCCTGGGCTCTGGCGGCTTCCCTCCCGCCCCCTTCCCGGCCCTCACCCCGCGGCCG |
| | | CTCTCCCGGGACACGCAAAGGTCTTCCTTAGGGCGTGCGCCACCCCCGCCAACCGCGCCCCAGACAGGGGCTGTAGAAAGTG |
| | | GGTCTTTGGGAGCCAGGATCTGGGAGCCCCTGCTAGGAGAAAGGCGCCTCCTCTAGGAGAAGGGACCAAGCCAAAGCTCTGA |
| | | GGCTCACCCGAGGCTGATCTGTGGCCTTCTCCCCGGTCGGCGGCATTCCCACCAGGACTCTCCCTAGCATGTGCACAGTGAC |
| | | GCAGAAACCGATGCCCAGAGACAGCCCCCAAAGGGCGCTTTGATTTATACAGTTGTAATTGGCTATAAACTTCTGCAAAGTG |
| | | CCCATAACCAGCGTGCCTGCCCAATTGGGGCATCAAGGAGCGAAGCAAGAAAAGCGGAAGCGCTGAGGGCTGGGGAAAGACT |
| | | TTAAAAGGGGCACTCAACTAGGACTCTCCCCACCGCACTTCCTTGCCACCTCTAGGCACACTCGAACACCAGAAGGGGCAGA |
| | | CCCAGAGTCAATGAGCCCCACAGTCCGGCAAACCTAGGATCTTTTTCAGTTGCACACACACACTCACAGACATCCCTGACAC |
| | | CCTTGAAAGGAAGCTTTGGGAGAAATGACCCCTACCTGGAGCGGCGGCTTAATTCCCACCAGGGTTTCCTCCCCGTCTCCCT |
| | | GCTCTCTCCGCAGACGCCTGCCCCTAGGCTTCTTGCCCCTAGCCTTCCAGCTTTTGCTTGAAATGACCCCTTCACAGGTCAG |
| | | AGGTTCAGAGACGGGGCGCGCCCCCGTCGCCTTAGCAGCTGGGGCTAGCACCTTCTTCCCAGCGACAGCAGCTCTCCCTGGT |
| | | CTGCCTGCTCGCTGTACCTTGAATTGGCCCGAGTGGCCAAACCCGGTCCCACACACAGCCTAGCCATCCTCCGACCCCCCCA |
| | | GGTCACTTCTCTTCTTAGATGGCTCAGATGGGGAAAGAGAGAGCGGAGATAAATGGCCTAGACCTAGCCCAAGAAGGTTCGA |
| | | GCCTCCCTCTCTCCTCGGCGGCCAGGAGCCACGCAAGGAATGCATGCCGCAGCAAGTGCCGGAGGACGCGATTCTGCCGGCG |
| | | TCCAGCAGTGCTCACGCCGACATTCCCCGGAGCTTCCCGAAGGTGTCCCGGGGTTGCGAATGAGGAGCTCTTGGAAAGAGGA |
| | | CGCCTGAACACGGTGTTGAGACTCAGAATATTCACTCCCAGGCTCAGTAGGTCAGAGAAGCCCCGACGGGCCAGAGGCCCCC |
| | | GAATTTGTCTCCGGGCCGTGCCCCTCCTGGCGCCGAGCTCCCAGACAGCCCGGGACGCCCCTGTGCGCACTGGACGCGCGAC |
| | | GCGCAGCAGCCACTGGCTCGACCCGCGCCTTCCCAAGCACCCTCCGAAGGGGCGCAGCCTCTGCTTACCTTGGCTGCCGAGG |
| | | GACTGCTGCGCCGGCTTCCGCATCCACTCGCACAGGTTCCGCCGCTGGCCGCCGGGAGACAGCTGCTCGGCGGCAGCGGTGG |
| | | CGGCGGGCCCAGGAGGGCCGGGGTTGAGCGTTTGCAGCAGCCCAGAAGCGCAGGAAGGCGCGGCGGCCGGGTGGTGCGGGTG |
| | | GTGATGCGGGTGGTGGTGCGGATGGTAGTCTGCGGGGCTGCTGTAGCCCATGGCTGCGGCCGGGGAGCCACCGTTGAGGCCG |
| | | TGAGCCACGGCGTTGGCGGCGGCCGCGGCGCCTCCGGGCGCGTAGCCATTCCAGTCCTCCCGGAGTGGGGCGCCATACGCTG |
| | | CCGGCCAGGATGGCCCCGGGGACTGCGCGCTGTCCAAGTTCGCTGCCGCTGCAGCTGCGGCCGCCACGTGGTAACCGCCGTA |
| | | GTCCGGGTACTGCGGGGGGCTGACGAAGTTCTGCGGCGCCAGGTTGAGGCCGCCAGAGTGGCGCACGGAGCTAGGGTACATG |
| | | CTCACGTCCTTGTCCAGGAGGTAGCTCACGTACATGGTGGCGAGGGTCCGGGAGCAGACCTCACCATGCTGCCTGGGGACCG |
| | | ACGCTGGAGGCTGCCGGGGGGCACGAAGGGAAAGGGGCGAGGGGACTCGAGGAGCGGCGGGTGGCTGCGCCCCAGCCCGCGG |
| | | TGCTCCGCTGGCTCCTCGCGGCTCTTCTGCCTCCGAGGCGGTCCCTCCCTCTGGCCTGCCTCCTCCCTCCCTCCCTTTCTTC |
| | | CTTCTTTCCTCCCACCTCCTTCCCACTAGGCTGCAGAGGCGGGGAAGACCCGCCACAGGCTGGCGTGCGGAGCCCCAGGCCG |
| | | GCGGCCTTCCGTGATTAACGAGTGTTTACAAGACTCTATTAGTAATGACACAGACACCAATGGTTGGAGACGTCGAGGCGCA |
| | | GCGCGCACTCTACGCACAACCCCTCGAAACATAATTTGCATTTTAAAAGATAAAGGGGAGGGAGGCTCGTGAGAGGGCAGCG |
| | | ACCTGACACAGCTAAATATTCAAACCTTTATTGTTAAGAGCTTC |
| 423. | CEACAM6 | GGGACTCTCTGTGTGGTGCTGACAGACCCAAGGCCCAGACACAGCAGAGGTCCGTGCTGGGGAG |
| | | GGCGGGTCGTCCTGTTATGGAACAGGGGTCCAAACAAGCTTGCTTCTCAGAGCATCTTCTGGGGAACTGAATATAAACAGAA |
| | | AGGGAAGAGGAGGAGGGACAAAAGAGACAGAAATGAGAGGGGAGGGGATAGAGGATTCCTGAACAGAGACCGCACCCATGAC |
| | | CCACGTGACCCTGGGAAATGCTTCTATCCCTG |
| 424. | CEBPA | AGGTTATCCTAAATACTAGAGTTGCCGGGCTCCCAGCTCAGCCCCAAGAATTCTCCCCTCCTCG |
| | | CAGGGAGAAGCCACCGCCTGGCCCCCTCATCTTAGACGCACCAAGTCCGGCGCAGAGGAAGGGAGGGGACACGCGGAGCAGG |
| | | CCAGGCTTTCAGGAGGCACCGGAATCTCCTAGTCCTGGCTCGCACGGCTCGGGCAAGCCTCGAGATCCGGCGACCCCAAACC |
| | | ACTCCCTGGGTCCCCGCCGGAGGCTGGCCCAGGGCGGTCCCACAGCCGCGCGCCTCACGCGCAGTTGCCCATGGCCTTGACC |
| | | AAGGAGCTCTCTGGCAGCTGGCGGAAGATGCCCCGCAGCGTGTCCAGTTCGCGGCTCAGCTGTTCCACCCGCTTGCGCAGGC |
| | | GGTCATTGTCACTGGTCAGCTCCAGCACCTTCTGCTGCGTCTCCACGTTGCGCTGCTTGGCCTTGTCGCGGCTCTTGCGCAC |
| | | CGCGATGTTGTTGCGCTCGCGCCGCACCCGGTACTCGTTGCTGTTCTTGTCCACCGACTTCTTGGCCTTGCCCGCGCCGCTG |
| | | CCGCCACTCGCGCGGAGGTCGGGGTGCGCGGCGCCCAGCCCCTTGAGCGCGCTGCCAGGGCCCGGCAGGCCGGCGGCACCGA |
| | | GCGCGGGCGCGGGGTGCGGGCTGGGCACGGGCGTGGGCGGCGGCGTGGGGTGACCGGGCTGCAGGTGCATGGTGGTCTGGCC |
| | | GCAGTGCGCGATCTGGAACTGCAGGTGCGGGGCGGCCAGGTGCGCGGGCGGCGGGTGCGGGTGCGGGTGCGAGGGCGGCGGC |
| | | GGCGGCGGCGGCTGGTAAGGGAAGAGGCCGGCCAGCGCCAGCTGCTTGGCTTCATCCTCCTCGCGGGGCTCCTGCTTGATCA |
| | | CCAGCGGCCGCAGCGCCGGCGCCCCGACGCGCTCGTACAGGGGCTCCAGCCTGCCGTCCAGGTAGCCGGCGGCCGCGCAGCC |
| | | GTAGCCGGGCGGGGGCCCGTGCGCTCCCCCGGGCATGACGGCGCCGCCGGGGCCCGCGGGCGCGCCCGGGTAGTCAAAGTCG |
| | | CCGCCGCCGCCGCCGCCCGTGGGGCCCACGGCCGCCTTGGCCTTCTCCTGCTGCCGGCTGTGCTGGAACAGGTCGGCCAGGA |
| | | ACTCGTCGTTGAAGGCGGCCGGGTCGATGTAGGCGCTGATGTCGATGGACGTCTCGTGCTCGCAGATGCCGCCCAGCGGCTC |
| | | CGGGGCGGCAGGTGGGGCGGGAGGCTGCGCGGGGCCCGCGCCCCGGGGAAAGCCGAAGGCGGCGCTGCTGGGCGCGTGCGGG |
| | | GGGCTCTGCAGGTGGCTGCTCATCGGGGGCCGCGGCTCCGCCTCGTAGAAGTCGGCCGACTCCATGGGGGAGTTAGAGTTCT |
| | | CCCGGCATGGCGAGCCTCGGCGGCCTCCAGCCTGCGCGGGGCGTCGCCGCCGCCCACCCGGAGACCCTGCTCGCCCGCGCCC |
| | | GCGCACCTCCGGGTCGCGAATGGCCCGGCCCGCGCCGGCCCAGCTTTTATACCCGGCAGGCCGCGTCGCCCCCTAGAGTCCG |
| | | AGGCGGCCTCTGTCCCCGGGCTGCGGCGGCGCGGCGCCTGCTGGGTCCTAGCGCGCGGCCGGCATGGGGCGGCGAACCAGCG |
| | | CGGCACAGCGCCGCGCTCCCCAGGCAGGCCGCGGCGCAACGCCCACCGCCTCCAGCGCGCCCAGCAGAGCCGCGGCGCTCGC |
| | | TCCAAGCTCCGCCCCCGGCCCGGCCGTCGCCCCCGCGCCCACGTGGTCGGTAGCGGGGGCCCCCTCCTCCTGCCTGCCCTAG |
| | | GCGCCCGTATCCAGCCACGGCCGGGAGCCCAGGAGTATCCCGAGGCTGCACGGGGTAGGGGTGGGGGGCGGAGGGCGAGTCT |
| | | TGGTCTTGAGCTGCTGGGGCGCGGATTCTCTTTCAAAGCCAGAACCAGGCCTGTCCCGGACCCGCGTCCCGGGGAGGCTGCA |
| | | GCGCAGAGCAGCGGGGCTGGGGCCGGTGGGGGGCCGTTTGGGACGCGCGGAGAGGTCCTGAGCGCGGTGGCTCTGCGTCTCC |
| | | TAGCTCTGATCTCCAGGCTACCCCTGTGATTCCGCGCAGAGGTACCTCTCGGAGGACGCCGGGGTCCCATGGGCGGCGCCGC |
| | | GCAGGGCGCTAGGACCCCGCGGGGAGCGGAGGCGGCCTCGGCCCGGGAGCCTGGAGGACCTGGCCGGTCGATCCGCCCGGGC |
| | | TGGAAAACTTTCTTTATAATTACTTCTCCAGGTCGGAGCGCGCGGCTTGCTAGGCGCGCGGGGCCGGCGCTGTTACCCGGCG |
| | | TGGAGTCGCCGATTTTTTTTCCTGCGGGACCGCGGGGCCCCCCAGACTAGCGGAGCTGGACGCCGGGGCGAGCACGGGGAGG |
| | | GGCGCACCGAGGGAGGAGACAAACTTAACTCTGGGGCCGGGATTCCGAGGCGGGGGCCGCAGCCCTCGAGGCCCGAAGCCAC |
| | | CGCTTCCTCCCCCGCCTCCCCATTCAGGTGGGCGCCAACGGCGGGAGCGAGGGTGTCCAGGCCGCCGGGCTGCCAGGTCCGA |
| | | GCACGCACAGGGAGAACTCTGCCCAGTGGTTCGCCGGGCGCTGTAGTCCCCGGGATCCTAGGGACCGAGGCGGCCAGGCCCT |
| | | GGGGCCCCTTGAGTGCGGCAGCTAATGCTCTCACCGCGGCGGGGGAAGGAGCTTGCCACCGAGACCCCCAGCCACGTGCGTC |
| | | CCTCGCATTCTTTACCGGGGCCGGGGTGGCGGCTACGGACCGTCAGCTGGGCCCAGATGGAGTCTTGGGAGCCCTCAAGTGT |
| | | CTCCTGTCCTTGCCCGCGCCGCCCCTCGCCACTGGCGCTGAGGCCTGACGCCGCCTGCGTCCCGGCTAGAGGCGCGCTTGCC |
| | | TACAGGTGAGGGAAGACCCCCTTCACCGACAGTGGCCTTAGGCCTGGCAAGGCGCCACGACCCGCCCAGGAGCCCCGGAGGG |
| | | GGCACAGCTAAAAACACCGCTGGAGAGCCCCGAGCTTCCACGACGATCGCAGTAAAGAAGCAGTTTCATCTGGGCAACGCAC |
| | | ACTGCGCTTTAATCAAGTTCCTATTCAACATAGTCCCAGTGATTAATAGCCCAACTGCTTCGTTTTC |
| 425. | CKMT1 | TTCCCCGCGGCTGCCCCCGGGAGACCGAAGAGGTGGCTGGGGCAGGCCGAAAGCAGAGGAAAGG |
| | | TTGGGCAGTGTCACCCTTGCACACCCACAGGGTAGACGGATCGAGTTTCAGCTTAAACCGAGGGGTGTGGGGGAAGGGTGTT |
| | | GGGTTCAGCTAGTCCACGTGGGTGCTGTCCTCCACTTGGTGCTGAAATCTGGGCGGCCACATCCCCGGGGCGGGAGGGGGCT |
| | | ACATCCCCGGCTTTAGACGCGCGAGTCTCAGGTCCCGCTAATTACCTGGCGGGTGCTGCCCACCCCTGCCCTCGCGCACCTA |
| | | GCGCGGTGGCAGGCGGGAAGGCGGGGCCTGGGGGAGCCCCACCCCTGGAGACTGCGGCTGGGGCCTCCCTCTCCTCCGCCCG |
| | | CCCGCCTGCCACTAGCTCATTGCGCCTCTCCTGCAGTCTGATTGGCACCGGCTCCCATTCCGGCTCCAGCCTCCAATCCGAC |
| | | CCCCATTTCGGCTGCAGCCTCGGACCTAGCTCCGGCCCTCGGTCTATCCGGTTGCATCCTCCCTCCCTGTTCCGGATCTTAT |
| | | CTTGCGCCAGCGCCTACTCCAGGATCCCGTAGCCAGACCTCAAGCCATGGCTGGTCCCTTCTCCCGTCTGCTGTCCGCCCGC |
| | | CCGGGACTCAGGCTCCTGGCTTTGGCCGGAGCGGGGTCTCTAGCCGCTGGGTTTCTGCTCCGACCGGAACCTGTACGAGCTG |
| | | CCAGTGAACGACGGAGGCTGTATCCCCCGAGGTAACAGTGCCTGAGGCGCGGGAGGAGGCGGGGGCAGGAGGTGATGGGAAC |
| | | GAAGGTGCGGGTAGAAGTGAGAATCCGGGCAACAGAGAAGGGCTATAATCACGAAGGCCCTGGAGCTGGAGGGCTGTGCAGT |
| | | CTGCAGACCTCAGTGGGGTGGGGGTGGGGGCCAAAACCATAAAGCAAGAACATTCCTGGGGACCTGCCAAGACCAGCTCTGG |
| | | CCCTACGAGTTCTAGCTGCACTGGCTGCCCAAATCCCTAATTGTA |
| 426. | CNN3 | AAACAAAAGCAACCCACAAAAATAATCCTTTATGTCGTGAAAGTCTAATGTACTTCAAACTTCT |
| | | CAGTGTTTCATAAAAAGTCTCAAGCCTAAGCAGATATCACTCGCACAGTGGCGCGCTCAGGCTTTGTCTCCATTTCCCGCAA |
| | | AAGCAACAGCAGAAGCAGCAGCAGCAATCTGCCTCCAAATGCCTCCCAGGCACCGAGATCGGAGACTGTGATGGGTCCCGGA |
| | | CCGGCTTCCGTTCCTCGGGGCCCCTGGGGTCGGCGGGACATTAGGCGGTCTCTCTCCCCGTGGGGAGGCGCGGAGTCCGCCA |
| | | GCACCCGGGGCCCGGGCAGATGGCGGGGGCTGCGGCAGCGGCTCGCCGGGTCCCTGGCCGCGCAGACGGGCTCCGCCTAAGG |
| | | GCGAGTGGCCACGCAGGAGCGCCCCCTTCCCGGAGGGCGCGTTCTGCAGTCACCAAACGGCCCCCGAGACCCCCGCAGCCGG |
| | | AAAGGCTGGCGCCGCGGGACTCCAGGCGCCCGGGAACCCACCGCCAGCCACTAGTCCTGTCCCACGGCGCGGGAACAAAGCC |
| | | AGGCGGGTGCCCGGGAGCCGGCCCCGCAAGCCCGGGGACTGGACGCGACCGGGACAGGCAGAGACGCTCGCGCCGCCTCGAC |
| | | GGCCCCTCTCCAGGAAAACGGTGAGCCACAGCGCGAAGAGCAAACGAAGCACGGCCCAGCGCCAGGCCAGCCCAAGGGTGCC |
| | | CCGGGGGCCCCCGCGCCCGCCCGAGCCAGGCGTACCTTGTTCTTGACTTCGGCCGAGAGCCCATAGGAAGGGCCCTTGTTGA |
| | | AGTGGGTCATGGTGGTTCGGGCGGCGGGAAGAGACAGCGCTGGGGTCCGGGGTCTCTCGCACTTCGCTTCCCCGCTCCTGGC |
| | | CCCGAGGAGTGGCCGCCGCGGGGGATGCTCGAACTCCCTCCTCTGGGAGGCGCAGGAGACGGCCGCGGGGCGCGGGCGGTGC |
| | | CTGGGCGACTGGGTCCAACTGGGTGCTACAGAGCCTCGAGCTCCGCTGCGAAGCACCCGGCTGCCTCGCTCGCCGCCCGCAC |
| | | CTCGGTTCCCCACAGGCCGCCCCCGCCTCCGCCTGGGCCTTCGAGGATTGGCCGCCGGGCCCGACCAATGGAGGGCCGCCTC |
| | | CTCCCGCCTCTGCGCCGAGGGGCGGTGCGGCCAATTGAAGGGCCGCGGGGGCCGCTTTCCCTCCCGCTCCCTCCCCGCGAGG |
| | | TCCCCGGCCCCCGCCCCAGCCCTCACGCCACCGGGGCCGGCGGCTAGGCGGCCCCGCCCCCGGGCTAGGCAGGGCGCCTGGG |
| | | GGCTTCCCAGCCACAGGCCATTCACGTCAACCTCCAGGCATCCTCCGCGGCGGGCCGGACCGGGCCGGCGGAGGGGAGCGGA |
| | | AGCCCACCGGGGCTGGCCCGCCGCTGACCCGCCGCCTCCGTCTGCCTCCTCCCGGGCTCTGGGCCCAGCGCGCCCAGATGCC |
| | | GGGCAGGACCCCTCCGCCCGCTGCTGGAACACCAATAGCAGCGTTGGAGCCTCTGGGCCGGAGGAACTGGGGCCTCCACCGA |
| | | CACCTGCTTCTTCCCTCACCTCGCAGCCTTCCACACCTGTTGATCTGACCTCTGCCCCGCTGCCGCCCAGGAGTAACCTAAG |
| | | GCTTTTGGCCTGATTCGAGCTCACTCCGTCTAAACCTCTAAATGCGGTGCGAAAAA |
| 427. | COL1A1 | CATGTAGACTCTTTGTGGCTGGGGAGGGGGTTAGCGTCCGCTCATGCGTGGCCTCACACTCCGC |
| | | GTGCCTCCTGCTCCGACCCCGAGGAGAAACTCCCGTCTGCTCCGACGACTGGCCCGGGCCCCTTTTATACTGTCCTGATGGA |
| | | GAGCAGGGAGGAACCCTGCCCCTCGGAGAGGGGGAGCCGGCCGCCCGTGCCCCAGCCAATCAGAGCTGCCTGGCCCGGCCCC |
| | | CAATTTGGGAGTTGGAATGGAGAGGGGGAGGAGGAGGGAAGGGAGTCCACCCC |
| 428. | CTNNAL1 | CTCAGGGCGTCGGTTAGCTGATTGGCAAATACAGTAACGCACACATCCCAAACGGGACTTGGCA |
| | | AAACGGTGTGCATCTGGATTAACTGTCATACAGTCTCCACGTCACTCTTCCACGAGGCCGGACAGAAGCGGGCAGAAGTGAG |
| | | CAGCAGCAGGTTCAGGGGAAGGGGTAGGGTGGCCGGGATGAATGGGAGGCCAGCGAACTGACCTACGGCGGGGAGGGACAAA |
| | | GGATCAGGGAGCGCACGCTGGGCGGGGAGGCCGGGAGGAATACCCCAGGCGGAGGAGCCGGGCTCGGGACCAGGCTGCGAAC |
| | | TGGTGACAAAGTCGGTTATCGTGGAGGGCCCTAAGTACCAGCGAGGGGCCCCGCGGGGGTCCTCTCTGCCTCGGGAGCTGGG |
| | | CTCAACCACTCGCCCCGCGGAGGGGGCATCGCCCGGCCGGGGGCACCCGGGAGCCGCAGACCCGAGGCAGGTACTGGGGGCA |
| | | AAGGCCCAGGCCGGCACCCCCGCCCCGCAGCCTCCCCTGCTGCCGAGTCCACTCAACCCTGCCGCCGGAGTTTGGATTATCC |
| | | AGTCTCTCGCCGCCCGGGCCGAGGCCCGTCCCGGAGCCCCACACAGGCGGTCCGACAAGAACGCCGCAGTGCCGGCGCGGAA |
| | | AGTGGGGCAGCGGCTGCCTCCCGTCCGGTCGTGCGAGCGGCGGCCGCCATCGCGGGCCGCGGCGGGAAGGAGAAGAGGGGCC |
| | | GCGCCAGGCGCCACTTTACCTGAGAAACCAGCGGGAGTAGCGTCTGCTCCACCGAGCGAGTTTTGATCTCCAGTCCCGAGTC |
| | | GAGGGCGAAGCCCGAAGAGCCGGAGCCGTAGACTGCTCCGGCGCCGCCAACGCCGGCGGGTCCGGGAGAGGCGGCCATGGCC |
| | | CTCGGTCTATCCCGCAGCCGGGACTCCGCGCCGCGGCGAGCCTGCCGCCAGTCAGCCCACCCGCCCGAGGCGGCGGCGACAG |
| | | GACACTCGCGCCAACCGAGACCCCGCCCCCAGGGGCCCGCCCCCCCAGGGCTCCGCCGCCAACCCTGCGTCCCCGACGCCCG |
| | | TCACCGTATGTCCCCGCCCACCCCCGGCCTCTGCCCCGCCCGCGCAGTCCAGCCTGGTGTGGGCTCCCTGCAACCCCACCCC |
| | | CTAAGCCGGGTCCCGCCCCCACATGGCCCCGCCCTTATCTCTTCTCAGACCCCTTCCATGGGTTCCAGGCACCTTCTCGCCA |
| | | GGGTGGCGAGGAGAAACAAAACACATCTGTCACACAGCCACATGGCCTCCTCTTACTTGGACAGACAAGCGTCCCCAGCA |
| 429. | D2S448 | TTCCAGCAGGAAGCGCTTGTTGCATGGCGCACAACAGGTTTCATGAGCTGGGGGTAGATAGCCC |
| | | CATGTCAGCGGCAGAGGTGCCAAGGGGTACCGGGCCCTCACACATCCTCCAGGGGAATTGAGTGCGTCTTGCTTTTCTCCAC |
| | | GTCCCAGGTTACCACTACAACGACCTGGTGTCTCCACAGTACCTGAACCTCATCGCAAACCTGTCGGGCTGTACCGCCCACC |
| | | GGCGCGTGAACAACTGCTCGGACATGTGCTTCCACCAGAAGTACCGGACGCACGACGGCACCTGTAACAACCTGCAGCACCC |
| | | CATGTGGGGCGCCTCGCTGACCGCCTTCGAGCGCCTGCTGAAATCCGTGTACGAGAATGGCTTCAACACCCCTCGGGGCATC |
| | | AACCCCCACCGACTGTACAACGGGCACGCCCTTCCCATGCCGCGCCTGGTGTCCACCACCCTGATCGGGACGGAGACCGTCA |
| | | CACCCGACGAGCAGTTCACCCACATGCTGATGCAGTGGGGCCAGTTCCTGGACCACGACCTCGACTCCACGGTGGTGGCCCT |
| | | GAGCCAGGCACGCTTCTCCGACGGACAGCACTGCAGCAACGTGTGCAGCAACGACCCCCCCTGCTTCTCTGTCATGATCCCC |
| | | CCCAATGACTCCCGGGCCAGGAGCGGGGCCCGCTGCATGTTCTTCGTGCGCTCCAGCCCTGTGTGCGGCAGCGGCATGACTT |
| | | CGCTGCTCATGAACTCCGTGTACCCGCGGGAGCAGATCAACCAGCTCACCTCCTACATAGACGCATCCAACGTGTACGGGAG |
| | | CACGGAGCATGAGGCCCGCAGCATCCGCGACCTGGCCAGCCACCGCGGCCTGCTGCGGCAGGGCATCGTGCAGCGGTCCGGG |
| | | AAGCCGCTGCTCCCCTTCGCCACCGGGCCGCCCACGGAGTGCATGCGGGACGAGAACGAGAGCCCCATCCCCTGCTTCCTGG |
| | | CCGGGGACCACCGCGCCAACGAGCAGCTGGGCCTGACCAGCATGCACACGCTGTGGTTCCGCGAGCACAACCGCATTGCCAC |
| | | GGAGCTGCTCAAGCTGAACCCGCACTGGGACGGCGACACCATCTACTATGAGACCAGGAAGATCGTGGGTGCGGAGATCCAG |
| | | CACATCACCTACCAGCACTGGCTCCCGAAGATCCTGGGGGAGGTGGGCATGAGGACGCTGGGAGAGTACCACGGCTACGACC |
| | | CCGGCATCAATGCTGGCATCTTCAACGCCTTCGCCACCGCGGCCTTCAGGTTTGGCCACACGCTTGTCAACCCACTGCTTTA |
| | | CCGGCTGGACGAGAACTTCCAGCCCATTGCACAAGATCACCTCCCCCTTCACAAAGCTTTCTTCTCTCCCTTCCGGATTGTG |
| | | AATGAGGGCGGCATCGATCCGCTTCTCAGGGGGCTGTTCGGGGTGGCGGGGAAAATGCGTGTGCCCTCGCAGCTGCTGAACA |
| | | CGGAGCTCACGGAGCGGCTGTTCTCCATGGCACACACGGTGGCTCTGGACCTGGCGGCCATCAACATCCAGCGGGGCCGGGA |
| | | CCACGGGATCCCACCCTACCACGACTACAGGGTCTACTGCAATCTATCGGCGGCACACACGTTCGAGGACCTGAAAAATGAG |
| | | ATTAAAAACCCTGAGATCCGGGAGAAACTGAAAAGGTGAGCTGAAGAGGCTGTGTGGGATGGCTCGTGTACCTAGGCACCTG |
| | | GAACACCTGAACTGTGGTGTGTGTGTTATTTGTCTCTGTGTGTGAGTTTTGTTCGTGTCGTGCAGAGGTGAGATGAAGGTCA |
| | | AGTCCATGAAGGTCAAGAT |
| 430. | DAPK1 | CGGCACAAGCTGGGATCCCAGTACACATCTCGGGACGGAAGAACCGTGTTTCCCTAGAACCCAG |
| | | TCAGAGGGCAGCTTAGCAATGTGTCACAGGTGGGGCGCCCGCGTTCCGGGCGGACGCACTGGCTCCCCGGCCGGCGTGGGTG |
| | | TGGGGCGAGTGGGTGTGTGCGGGGTGTGCGCGGTAGAGCGCGCCAGCGAGCCCGGAGCGCGGAGCTGGGAGGAGCAGCGAGC |
| | | GCCGCGCAGAACCCGCAGCGCCGGCCTGGCAGGGCAGCTCGGAGGTGGGTGGGCCGCGCCGCCAGCCCGCTTGCAGGGTCCC |
| | | CATTGGCCGCCTGCCGGCCGCCCTCCGCCCAAAAGGCGGCAAGGAGCCGAGAGGCTGCTTCGGAGTGTGAGGAGGACAGCCG |
| | | GACCGAGCCAACGCCGGGGACTTTGTTCCCTCCGCGGAGGGGACTCGGCAACTCGCAGCGGCAGGGTCTGGGGCCGGCGCCT |
| | | GGGAGGGATCTGCGCCCCCCACTCACTCCCTAGCTGTGTTCCCGCCGCCGCCCCGGCTAGTCTCCGGCGCTGGCGCCTATGG |
| | | TCGGCCTCCGACAGCGCTCCGGAGGGACCGGGGGAGCTCCCAGGCGCCCGGGTGAGTAGCCAGGCGCGGCTCCCCGGTCCCC |
| | | CCGACCCCCGGCGCCAGCTTTTGCTTTCCCAGCCAGGGCGCGGTGGGGTTTGTCCGGGCAGTGCCTCGAGCAACTGGGAAGG |
| | | CCAAGGCGGAGGGAAACTTGGCTTCGGGGAGAAGTGCGATCGCAGCCGGGAGGCTTCCCCAGCCCCGCGGGCCGGGTGAGAA |
| | | CAGGTGGCGCCGGCCCGACCAGGCGCTTTGTGTCGGGGCGCGAGGATCTGGAGCGAACTGCTGCGCCTCGGTGGGCCGCTCC |
| | | CTTCCCTCCCTTGCTCCCCCGGGCGGCCGCACGCCGGGTCGGCCGGGTAACGGAGAGGGAGTCGCCAGGAATGTGGCTCTGG |
| | | GGACTGCCTCGCTCGGGGAAGGGGAGAGGGTGGCCACGGTGTTAGGAGAGGCGCGGGAGCCGAGAGGTGGCGCGGGGGTGCC |
| | | ACCGTTGCCGCAGGCTGGAGAGAGATTGCTCCCAGTGAGGCGCGTACCGTCTGGGCGAGGGCTTCATTCTTCCGCGGCGTCC |
| | | CTGGAGGTGGGAAAGCTGGGTGGGCATGTGTGCAGAGAAAGGGGAGGCGGGGAGGCCAGTCACTTCCGGAGCCGGTTCTGAT |
| | | CCCAACAGACCGCCCAGCGTTTGGGGACGCCGACCTCGGGGTGCCGTGGTGCCCGGCCCCACGCGCGCGCGGGGCTGAGGGG |
| | | TCGGGGGCGTCCCTGGCCGCCCAGCTTTAACAAAGGGTGCTCCTCTCCACCCCGCGAGGAGGGGCAGCTCCGGAGACCCGGT |
| | | CTTCAGCGAGCGGGGTCTTAGCGCCGGGGAGGTCTACTTCCTTTTGGGGTTGCCATTTTACTATTATTATTGCCTTTTTTTT |
| | | TTCTTCAAAAGGACTGGAGACTGATGCATGAGGGGGCTACGGAG |
| 431. | DLK1 | GGCTTTTCCTGGGGGTTTTTCTGTGTGCGTGTGTAATTATGTGCTTAGTTCACATCTTCATAGT |
| | | GCGCCTTTGTGTTTTCCTGGGTATCTAACCATTGCACGTGTGCCCGGGACTTCAGCGATAAGTGTTTCGGTGTTCCTGCGTG |
| | | CGGATTTGTGCTCTCCGGGGAGGTCTGCGGCCCAGGTTCGATTCCTGCGACTTGTCCTAGGCAGGCCTGTATGTGCGCGGCG |
| | | GCCGCGTGCTGTACAGTGTGAGGGAACGTGTACCAAACGCTCGCGGGATACCTGTGCCCGTCTAGCCAAGAGTGCACCCGTG |
| | | TGCGCGAGCGGGCTTCTGGGACGCCGCCGTGGTCGGGGGCGGCCCTGCGAGGGGAGGGGGTCACAGGGACTGGCCGGCGCCG |
| | | GCCCCGTGCGCACGGAGGCGGGGGCGGGGGGCGGGGGCCGCGAGGGGGGAGGCGGTACGAAAAGGGCGGCGCGCGCGGCGGC |
| | | GGCGGCAGCTCCCCGGCAGCGGCGGTGGAGAGCGCAGCGCGCAGCCCGGTGCAGCCCTGGCTTTCCCCTCGCTGCGCGCCCG |
| | | CGCCCCCTTTCGCGTCCGCAACCAGAAGCCCAGTGCGGCGCCAGGAGCCGGACCCGCGCCCGCACCGCTCCCGGGACCGCGA |
| | | CCCCGGCCGCCCAGAGATGACCGCGACCGAAGCCCTCCTGCGCGTCCTCTTGCTCCTGCTGGCTTTCGGCCACAGCACCTAT |
| | | GGTGAGTTCCCCGGCGGCCCGGCTCGCGCCCCCTCTGGGGAAGCCTGCGACTCCCCGCCGGCCGCCCGGTGCCCCGCACGCC |
| | | CCGTCTCGTGAGCCCCAACTCCGCCCGTCCCGCCTAGCCCTAAGCC |
| 432. | DMPK | AGAAGCGCTGGGGAGGGAAGAGGCCCTTGCTCTCCCCCACCTCGGCCTCGGGTCCCCAGAGGCT |
| | | GAGCTCCCAGGTTGCCCTAAAAGTTCCTTGGTTGGGCGGAAAGCGGGCGAGTGGCCTGGAGAGAGGGAGCAGCAGCCGCCAG |
| | | CCCAGTTCCCGGTGCCCTCCCCGCGGGGCGTCAGAGAAGGGAGAGGCCGGCGCTCAGGGTGGGAGGAGAAGGGTTTGGGTTA |
| | | CAGGGAAACCGGAGCTGGGAAAGGTTCACGTTTCACAACAAAGGCAGAAGACGGACCACGCCGTCCGGGCCCGGAGGGAGTG |
| | | TGGGGGCGGGTGGGTAAGAGTAACGGTCAGTGAAGAAAGGGGGCTGGGAGGCAGCCCCTACGCGGAGTGGAGTGGCCACAGG |
| | | CCCTGTCCTTTTTCCTCAGTCCCTCTAGTGCCCCCCGCAGTGTGATTCCCCAACACCGATGGGATTTGGGAAGGAGCTCGGA |
| | | ATGGAGCCGCTGGAAGAGGAGACGCGTGCGGGGAGAGGGCCCGGGCGGGTGCCTGTCCCGTCCACACTTAGTCCCCGCGCCC |
| | | CGCGGGCGCCTGAGATTGTGAGCTGGTCCCGGGAGATGTCCGAGGACCTCGGCGCGCCGGCCCCAGCAGTGGGCACGGGGGA |
| | | AGAGGGCTGGTGGACGGGATGTCCCCGGGAGAGCTGGACTTGCGCCGCCCGAGGCCCCTCACCTCTTGCAGGGCGCGCCGCC |
| | | TCCGGCCCCGGTCCGGTCGCGCTGTCGCCGGTTCTTGAACCAGTTGCTGACCTGCGTGAGCGACAGGCCGGTGAGTGTGGCC |
| | | AGGCGGCGCTTCTCGTCCGGCGTGGGGTAGCGGTTGCCGCGGTAGCAGGCCTTGAGCGCTGCGCGGGAGCGCTCCTTGAAGC |
| | | AGTAGACTGTCTCCTCGCCGTCCCAGATGGTCTTGGGCAGCGGGAACTTCTTGCGCAGTCGATACTTGTCCACTGCGCCAAG |
| | | CGCGCGGCCGCGGGCCCGCTCGGCCTCATGGTAGCGCGCGCGCAGGTAGAGGTCCTGCAGGAAGGCGTGGTGGGCGGCGGGG |
| | | AAGGGGCGGCTCTCGAGTAGCCGGTAGAGCTCGGCGTACTCGCCCCGCTGGAAGGCCACCAGGGCCCGCGCGCGCAACACCG |
| | | GGTCGCTGCCACGTAGGCGCTCGGCCGGGGGCAGTGCGCCCAGGAAGCGGCTCAAGCGGCCGGCGTGGCCCGCCTGGAGCAG |
| | | CGCCTCGCAGACGCACGCCACCTGCTCGGGCGAGAAGCGGAGGCCCGTGGGCGGTTCGGAAGCGGCCTCGGGGGGCGACCCG |
| | | GGGACGCCCGGGGATCCCGGGCCCTCAGCTCCCGCAGCCGCTGCGCCCGCCCCGGCCCCGGCCGCCGCCGCCGCCTCACCCT |
| | | CGGCCGCCTGCAAAGTCTGCAAGAGCTGGCGCGCTTCCTCCTCCTCCTCTTCGGTCGCCGCCGCCGCCGCCACCGCCTCCCC |
| | | CCCAGCCGCCGGCCCCGCGCTCGGCTCCGCAGGCAAGGTAGCCATGTTTTGCAACTTTGGGAAGTTCCTCCCTCCCTCTCTT |
| | | CCTCCCTCGGGCTTTCCCCAGCCTCCTCCCCCACCTGTCCCCCCTTTTCGCCCCCACTCCCCGCTCTTCTCGATCTTCTTTC |
| | | TGGCCGACCCTGCGCCCCACGCCGGGAAGGCGAGATCCAGCTCTCCACTCGGGTCTCTGTCCCCTTGTGTGTGTCCGTCCCC |
| | | CTCCCGTCTGTCTGTGATTCTCCCTTTGTTTTCCCTCCGCCTCTGGCCGCGCTTTCTGCCTCCCCCCAGCGTGTGCTTCTGG |
| | | CTCAGGGCCTCAGTTTCCCCATCGGGACAACGCAGAAGGTAACGGGCCGTCCAGGAGGACTAAGGGCGCGAAGCCTCCGCCC |
| | | CGAGACTGAGCTTCTGCACGCCTCCGTCTCCAGGGTCCTCTGCAGGCCCCCACATTCCCCATCTCGGCCTGCGCTCCGCCCC |
| | | TCGGAATTCCCGGCTCCGCAGGGGGGGCGGGTCTGGCCGGGAGGAGGGGCGGGGAACGGGCTAGAAAGTTTGCAGCAACTTT |
| | | TCTCGAGCTTGCGTCCCAGGAGCGGATGCGCGTGGCGTGCGCAGGCGCAGTGGAAGGAGGATGGCCGCGCGCGCTGCCAGCC |
| | | CAGCCCCCTCTTCTCGACGCTCGGTGGCACAGCTGGGCCACAGCTGGGCGGGGGCGGTGCCTCCGGGTGGCCCGCTCGCCCT |
| | | CCTATTGGCCGGACGCCAAAGCCCCGCCCCGTGGCTTTTCCTCCCCCAACCCTGATTCGGCCGCTTCGCATCCCGCTAGCTC |
| | | CTCCCAGACCTTCGGCCGCCTCCACACGCCTCCGGATTGGCCCGCTGCGGAGCCTCCGGCCCACAACGCAAACCGCGGACAC |
| | | TGTGGAGTCCAGAGCTTTGGGCAGATGGAGGGCCTTTTATTCGCGAGGGTCGGGGGTGGGGGTCCTAGGTGGGGACAGACAA |
| | | TAAATACCGAGGAATGTCGGGGTCTCAGTGCATCCAAAACGTGGATTGGGGTTGTTGGGGGTCCTGTAGCCTGTCAGCGAGT |
| | | CGGAGGACGAGGTCAATAAATATCCAAACCGCCGAAGCGGGCGGAGCCGGCTGGGGCTCCGAGAGCAGCGCAAGTGAGGAGG |
| | | GGGGCGCGGGATCCCCGAAAAAGCGGGTTTGGCAAAAGCAAATTTCCCGAGTAAGCAGGCAGAGATCGCGCCAGACGCTCCC |
| | | CAGAGCAGGGCGTCATGCACAAGAAAGCTTTGCACTTTGCGAACCAACGATAGGTGGGGGTGCGTGGAGGATGGAACACGGA |
| | | CGGCCCGGCTTGCTGCCTTCCCAGGCCTGCAGTTTGCCCATCCACGTCAGGGCCTCAGCCTGGCCGAAAGAAAGAAATGGTC |
| | | TGTGATCCCCCCAGCAGCAGCAGCAGCAGCAGCAGCAGCAGCAGCAGCAGCAGCAGCAGCAGCAGCAGCAGCATTCCCGGCT |
| | | ACAAGGACCCTTCGAGCCCCGTTCGCCGGCCGCGGACCCGGCCCCTCCCTCCCCGGCCGCTAGGGGGCGGGCCCGGATCACA |
| | | GGACTGGAGCTGGGCGGAGACCCACGCTCGGAGCGGTTGTGAACTGGCAGGCGGTGGGCGCGGCTTCTGTGCCGTGCCCCGG |
| | | GCACTCAGTCTTCCAACGGGGCCCCGGAGTCGAAGACAGTTCTAGGGTTCAGGGAGCGCGGGCGGCTCCTGGGCGGCGCCAG |
| | | ACTGCGGTGAGTTGGCCGGCGTGGGCCACCAACCCAATGCAGCCCAGGGCGGCGGCACGAGACAGAACAACGGCGAACAGGA |
| | | GCAGGGAAAGCGCCTCCGATAGGCCAGGCCTAGGGACCTGCGGGGAGAGGGCGAGGTCAACACCCGGCATGGGCCTCTGATT |
| | | GGCTCCTGGGACTCGCCCCGCCTACGCCCATAGGTGGGCCCGCACTCTTCCCTGCGCCCCGCCCCCGCCCCAACAGCCTACA |
| | | GCTGTTGTTAGTCCACTCGCACGCCTCGAATCCCGTCCGAACTCGTCATTGGCTGCTTCCTAGCGGCCTGTGTTGATTGGCT |
| | | GCCCGAAGATCCGCCCTCCTGCCGTGGGCCCAGCCCCGCAAATGCGCAGCTAAGCGGGTGGCAAGGGGCGGGTGGAGCGCGG |
| | | GGCGCGACGGCGGAGGGGGGCGTGGGCAGCCGGACGTACCCTGGCAGGGAGCAGCAGGTGGCGGCGGTGCATGGGGCCTGGC |
| | | CCCACCAGCGGGCACTGGCCCACAGCCACGGCCGGGGGGCCATCTAGCTGGAGAGAGAAGGGACAGGTGACCCGATCGGAGC |
| | | CCAGCCCAGCCCTCAGCGGTGGGGCGAGAGACAGCGAGGGGAATCGAGGTTGGGGAGGTTATCTAGGGAGATCCCGGAGGGA |
| | | ATCTGGTGAGGCCTGAACGGAGGGAGATCTGGGGCTGAATAAAGGGCTTCTGCCCTCTAAAGTCGCAAAGACGTAGGGTGAG |
| | | CCCTATATCTGGACGGGGAGACCAGGAGCCAGGGAGGGGATCTGCAGAATGGGCAGCAGGTCTGAGGCAGGGGAAAGAGAGG |
| | | GGTCTTACATGGGAAGGTGGATCCGTGGCCCGGGGACTGGGGACCCCCGTGACAGCTGGAAGGAGAAGAAAGAGGCATAGGG |
| | | CGCGTGGAGGGGCGAAGGAGGGCGGTGGCGCGGCGTGCCCCAGCGTGGGTCCCTTCCCTCCTCCAGGTGTCTATACACGCCC |
| | | CGCGGAGCAGACGGCCCACCTCCTCCCGGTCCTCCGGGGAAGGGGACACATGAGGGACTCACCTGTGGCTCCCTCTGCCTGC |
| | | AGCAACTCCATCCGCTCCTGCAACTGCCGGACGTGTGCCTCTAGGTCCCGGTTCCGAGCCTCTGCCTCGCGTAGTTGACTGT |
| | | GGGGAGGTAAGGACGGTGAGTCCGTCCGGGCCGGACGAGAGGGGATGCCAAGGGTTGCCACCGGCCCGCATCCCGGCCCCGG |
| | | CCCCGGCCCCGATCCCGACCTGGCGAAGTTCTGGTTGTCCGTGCGGATGGCCTCCATCTCCCGGCTCAGGCTCTGCCGGGTG |
| | | AGCACCTCCTCCTCCAGGGCTTCCTGGAGCTCCCGCAGCGTCACCTCGGCCTCAGCCTCTGCCGCAGGGACAGCCGCTGGAA |
| | | CTGCCACTTCAGCCTGTGTATGGGGACCAGGCTTAAGGCTGCCTGTGGCTCCTGGAAGACTCAGGACTTGGGCACTGGTTCC |
| | | AGGCTAGGAATC |
| 433. | DPEP2 | GCCCAGCGTGGTGAGGGCTCTGGGGGGGCCTGGCGTGGTGTAGGCACAGGTTACAGGCTGGAGC |
| | | AGCAGCAGCAGGAGCAGCAGACTCAGCAGAGGCCACCGACCAAACGTGCCGGGACCCTCGAGGCCGGAGGGCTGCATGTTGT |
| | | GCAGGGCCGGGCAGGCAGGCAGGGGTGGCAGTCAGAGAGCCTGAGAGGGGCGGGCGAGGGGCAGAGCGCGACGATGGAGTCC |
| | | CGACCCTCGATTCAGGCCACCTGGCGGCGTGGCCTCAAGAGGAGGGTAACGAAGTCACAGGGCCCCGCGAAGGATGTGCGGA |
| | | GAGCGGCGGCACCGCCCAGTACGGCCACCAGGGGGCGCCCAGCCCTCCCGCCGTCATCCCCAAGGGCGCTCCCGCCGGCTGG |
| | | GCGGATCCCTGTCCTGGGCGCCCACTCGCGGCCTGTCTTAGGGCCCTCCACCCTCCCTTTTACTCCTCCCACTGTCCCCAGG |
| | | GGGCCGACCCAGGTGGCTCCTCTGCTGCCCCTAGGCTCTCTCTCTAAG |
| 434. | DUSP4 | AAATAATCCCTCTCTCCCCGGGAGCGGGGCCTAAGTGGCTGCACCCCCGGAAGCCCAAGGGCCG |
| | | AGGTCCTGGAGCTGCCGCTCTAGGGCGGTCCCCTCGGGATTTCTTCCAGGAAGGAAAGCAAAACACAACAAAACAAATCCAG |
| | | ACTCCACGTGGGGCTGGAACCCAGTCCCAGCCCTGCTGTGTTCTAGCTGGTCCCGGGTTTGGACGCCAGCGAAGGAAGGCGC |
| | | GCCCGCGCTCTCCATCGGGGGGACTCGAGGGCAGCGCCGTGCGGGGAACATCCCGGCCAGCCCCGGCCATTCGAGGGGACTT |
| | | GCGTCCCAGAGGAATTGCCCTCATCTCTCCCACCAGCGCCGACAAGCTCAAACAATGGCGCGGACGCCCACCCTGGTGGCCT |
| | | AACCAGGACCTGGCCCCGCTCAGCCGCCAGGCCTTCCGGGACAAGCCCCTTCCTGGCACTTTCTCTTGGAGCGCTTTGGGGA |
| | | GAAGGGAGGGGGACTGGGCTCATTTGGAGGAGCAAAAGACAGCCCTCGCGGAAAAAGAAAGAAAAATCAGCAAAATCGCCTT |
| | | AGCAGTTCAACCAAAGGTCAACAGCAGCGTTTCCCCAGCAGCTGACAGGTCAAATGGGCAGGAAACAACTGCGGGGAACAAA |
| | | GCCCCCCGCTGTCCGGCAGGGGCGAACCCGGCTCCTCCGCACTGAACAGTTTTGTTGTGCTTTTTGGAGGGGAGAGGTTTCC |
| | | GCCCCCTTTCCAGCTCCCTTCCTCCCGGGTGGACAGGTCGGAAGAGGAGCGGAAAGGAAAGGAAGGGAGGGAGGCGGCGAAG |
| | | GCGCCTGCGGGGGGGAGGAGCGGCTCTTTGATGGGTGTGGGGGGGTCTGAACTGCCTACCGGGCTCGGAAGCGCAGACCCTA |
| | | CCCGCGCGGAAGAATGCGCGGAATGCGGCGGCGGGAGGCGGAGGCGCAGCGCGGCGGGGGGCGCCCGGACCCCACGCGCCCT |
| | | GCCCGCTCCCTAGCCCGAGGGCCGCGGACCCACCCTGCCCTGCAGGTGTGGCGGGCGCCAGGTCCCTCCAGGGACTCGGACA |
| | | GCACGCGATTTTTCCAGGATTTCCGACTACCAAATGTGACAAGACAGTCTCGCCGCCGAGAGTTTCGCCAGGGCTCGCTAAA |
| | | ATCCGGCACGCGCCGCCATCAGATAGTCGAAAATCCGGGCTGAACAAAAGGTGATCATTCTCCGCTGCCCGGATTTGCAGGC |
| | | GCTAGAGGACCGGTTCCGCCCGGCGTCCCCCAGATACCCAGTCCTTCAGCCTCCCGCTCCCAGAGCCTGGATCTAGCCCGGG |
| | | ACGGGGATTGGCCACCTTCCAAGCTGAGCTTGGAGTTGGGCAGCGGGAACGCAGATACAGATCGCTCTACACAGTGTGTGCA |
| | | ACCCAATCTCCCTTTCTGCATTTGCCAGGGTCTTGTTTTCAGTGAAACTGGCAAGGCCAATTGTACCTCGGCGCCCACCCCT |
| | | CTTCACCTTTTCTCCACCTCTGGGATTCTCCCGGTCCGGCTCAGTGTAGGGCCCCAGCACGCCAAACCAACAGGCCTATCTT |
| | | GTCCCCACGCGCCCCGTGGTACTTGGAAACTGTTCCCGGGACTCAAGATGGACCGCGCAGAGGAGTCGCTTCATCCTCGAGG |
| | | CTAGGACCGTTAATGTCAATAAACGGGGCCTCGCTAGGACGGTGCGGAATTGCAGGTGACCGGAGAAACCCGTCCGCACTTT |
| | | CCCTGAGGACTTGAACCTCGCACTTGCACAGAAACCTTGCAAACCCCTACACACCAACACACACAAAGGGGCGCGCGGGGAT |
| | | CCCCGCAACCACGAATCACGCCGGGGACTCCTTCCCGTGCCAATAAGGCGCAGGCCGCCAAGACCCCCTCCATCTCCCCGAC |
| | | TCCAGCTAGCGCCCGGAGCCCTCGTTTACCTTTGAGCAGGCAGATGTCGGTGCGCTCGGCGTTGCGGCGCAGCGCCTGCACC |
| | | ACCAGCGACACGGTGCTGTCCTCGCGGAGGCTCTCGGCGCGCGGGCTGCGCTCGTCGTAGACGATGACCGCCGAGTAGAGGC |
| | | CGGAGCGCAAGCGGGCGCGTACCTCCTCCTCGGCGGGCAGGATCTGCTCCAGGCTCACGGAGCCCTTAGCCCGCCGCCGCAC |
| | | GATGGTGTTACAGCGCACGTTGACCGAACCTAGGATGTAGCCCGCGCTGTGCGCCAGGAACGGTCTGCAGTCCAGCAGCAGG |
| | | CACTTGCCGCCGCTCGGCAGCCCCAGGGTGCCGTGGCTGCCGCTGCCGCCCGCGCCGCCGCCATTCTCGTCCCGGTTCATCA |
| | | GCCTTTTGAGCACACTGCAGTCCATCTCCCGCAGCTCCTCCATCGTCACCATGGTCGCCGGGAACCGAGGCGGCTGGGCGCG |
| | | CGAGGAAGAGAAGAGAACCCGGGCCGCCTAGGCTGCAGAAAGGGGCGGGCGAGAGCTAAGAAGGGACGCCTGCAGAAGTGGC |
| | | CGCAAACTTGGTCCTCAAGGGCTCCCGCGGGAGAGCCTCTTCTTCCCTGTCCCCTTCCTCCCGCAGCCTCGCGGTCACATAG |
| | | CAGTCGGAGCGGCCTCGGGCGCCCAGCCGGGCGGCGCGCAGAGCGGAGGGGGAGGCGCCGGTGGAGGAGAGTGTGTTTACGA |
| | | GAGAGGACCGCGGACTCTTTTCGGCGACGGCCTCCCGTGTATTTTTGCCGGTCGCGCGGCTCCTGTCGCCACTGGCGCCAGC |
| | | GCTGCCCTGCCTACGCTCCTCCGGCGCTCAGCGCACTGCCCCAGCCAGAGTTTTCTCCTCGGCTTAGAGGTTTATTAATGCT |
| | | CCTCCGCGCTCCCGGGGGAGGGGGCGCTAGGTACTACCCCGCCAGGCCCCGCCCCGTTCCATTCCGGGCCCCGCGCCGTCCC |
| | | CCGCCCCCCGCGCTGTCCCCCGCCCCCCGCGCTGACTCCCCGCCCCCTCCGGGCCCCTCCTCCCACCCCCTCCCCGGAGCTT |
| | | GAATGGAGCGCGGCCGCTGGCTCCCGGTCACGGGAACTCGACGTCACAAAGAGCGGAGTAAACAGGGCGCCGGGTGCTCGCC |
| | | ATCAGGCCCATTCATAAAACCGAGGCCGAGAGGAAGAGGGAGGCGGCCTCCGACCCGCCGCCAGCGGCGGGCAGCGCCCGGA |
| | | CTTTGTGAATGGAGCGCGCGGCGCCTTTCGCCGTCTACCCGCGGGCCCAGCCTGCGCCGCGCCTCCAGCCCGACACCGGCAC |
| | | CTGTGCCGCCGCTGCACCCCGCCGCTAGGCGGGCGGCGCCAAAGGAAATAGCCGGCTGAGGAGGCCAGGGCTCGGGTTTGAG |
| | | CTTTCATCCCTTGGCACCTTCGCGCTTCCCCGCGTGACAGCCCCGTAGGTTTTTCCCGGTTACCCAAGTGGAGAGGGGACCG |
| | | GAGCGCTGCTGCCCACCCAGCGGGGCCTGACACCTCCTAGCCCCAGACTCCCCGTGCGCTTTCGGCCCGGCCAGTCCTTTCT |
| | | GAATATGCGATTCCTGAGACGTTAGCGTATGCATTAGCGTGATCGATTTTCATCTAAAGCCATTAAATCAGTTTTATAGCTT |
| | | CCCCAAGAACGTGGGCAACCCCCTGCTTGGAAGATGAAAGAGAAAGTCCTCCCGCCCGAAGCCCAGGATCGGAGTGAGGTGT |
| | | GGGGTTTGTTGCTTTACTACCCAAAGGGAGGCATTAATTGCCGGAATGGTGTTCCGAGGGAGGAGAGCAGGCTGCGCTCCTG |
| | | TCCACGCTTCCTAAAAGATGTTTATTTAACCCTGGTCGGGGGAAAAACAAGGTTTTCGGGTTTGGGAAACGTCTTCACCCTA |
| | | TCTTGCATAGTTGTGAATTATCCTTTCAGGAAATGACAGGGTCCACATTTGTTGCGGAGCTGGACCCTGGAAAATGTACCCG |
| | | CAAAGAATGACCTTTTGAAAGGACTTCAGCAGCTCTGGGTCCGATTCATGTTTCTGGGCATCCCTTTTTCCTGGTCACGTAG |
| | | CAGCCCTCCCCATCCCCATTTCTCTTTTGCATTTAATTTCCCAGAGGCGGCTTTTGGCTGGGGGTTTGTGCACGCACGCGGT |
| | | GGGGACCGTGCAGAAGCCAAAAATCATTCGCCCGACATCTTGCCAAGTCTTGTTAGTGAGAATTAATCACAGAGCAAGGGAG |
| | | TTACAGGACTCGCTCGCAGTTTCGGAGGCTGCAGAGTAGCGTATTCCATTCCGGGGCGCGCGTGGAAGCCCGACCCAGGCAG |
| | | CGAGGGCACCGGTACCCGCCGGGTCTCTCCCGCCCAAGCTCACCCTCTCCCCATGCCTTTGTTCCGGGCGGAGCGGCTTTCC |
| | | CGAGCTCTCACCTCCGGTCCCGGGGGCCATCCCTGCACTCCCCGACCGGAGCTCGGCGGAGCCTCCACTTCCGAGACCTTCG |
| | | ACTGGCCTCGTCCCCCACTGGGCCCCGAATGCCTCGACCCCCGGGCCCTACTCGGATGAGCTGTAGGGGCACCGCTTGGGCA |
| | | AGGTGATATTTCCTTTTGCTCCGGGCTCCAAAATGGCTTACGATGGCACTGCACGGATCCTTTCTCTTTTTAAAATTTAGTA |
| | | TTTTATTCATCATGGATTTTTTGCATTTTTTTTTCTTTTAAAGAGGCATTGCATCAAGATATTTCTTTTGATTACTGACTTT |
| | | TCGGCGCC |
| 435. | E. cad | GTGAGCCACCGGCGGGGCTGGGATTCGAACCCAGTGGAATCAGAACCGTGCAGGTCCCATAACC |
| | (CDH1) | CACCTAGACCCTAGCAACTCCAGGCTAGAGGGTCACCGCGTCTATGCGAGGCCGGGTGGGCGGGCCGTCAGCTCCGCCCTGG |
| | | GGAGGGGTCCGCGCTGCTGATTGGCTGTGGCCGGCAGGTGAACCCTCAGCCAATCAGCGGTACGGGGGGCGGTGCCTCCGGG |
| | | GCTCACCTGGCTGCAGCCACGCACCCCCTCTCAGTGGCGTCGGAACTGCAAAGCACCTGTGAGCTTGCGGAAGTCAGTTCAG |
| | | ACTCCAGCCCGCTCCAGCCCGGCCCGACCCGACCGCACCCGGCGCCTGCCCTCGCTCGGCGTCCCCGGCCAGCCATGGGCCC |
| | | TTGGAGCCGCAGCCTCTCGGCGCTGCTGCTGCTGCTGCAGGTACCCCGGATCCCCTGACTTGCGAGGGACGCATTCGGGCCG |
| | | CAAGCTCCGCGCCCCAGCCCTGCGCCCCTTCCTCTCCCGTCGTCACCGCTTCCCTTCTTCCAAGAAAGTTCGGGTCCTGAGG |
| | | AGCGGAGCGGCCTGGAAGCCTCGCGCGCTCCGGACCCCCCAGTGATGGGAGTGGGGGGTGGGTGGTGAGGGGCGAGCGCGGC |
| | | TTTCCTGCCCCCTCCAGCGCAGACCGAGGCGGGGGCGTCTGGCCGCGGAGTCCGCGGGGTGGGCTCGCGCGGGCGGTGGGGG |
| | | CGTGAAGCGGGGTGTAGGGGGTGGGGTGTGGAGAAGGGGTGCCCTGGTGCAAGTCGAGGGGGAGCCAGGAGTCGTGGGGACG |
| | | ATCTTCGAGGGAAGGAGAGGGGCATCCGTAGAAATAAAGGCACCTGCCATGCCAAGAAAGGTCGTAAATAGGAGTGAGGGTC |
| | | CCGGGGATAAGAAAGTGAGGTCGGAGGAGGTGGGAGCGCCCCTCGCTCTGAGGAGTGGTGCATTCCCGGTCTAAGGAAAGTG |
| | | GGGTACTGGAGAATAAAGACATCTCCAATAAAATGAGAAAGGAGACTGAAAGGGAACGGTGGGCTAGGTCTTGAGGGGGTGA |
| | | CTCGGCGGCCCCCTCCCGGGAGTTCCTGGGGGCTCGGCGGCCGTAGGTTTCGGGGTGGGGGAGGGTGACGTCGCTGCCCGCC |
| | | CGTCCCGGGGCTGCGGGCTGGGGTCCTCCCCCAATCCCGACGCCGGGAGCGAGGGAGGGGCGGCGCTGTTGGTTTCGGTGAG |
| | | CAGGAGGGAACCCTCCGAGTCACCCGGTTCCATCTACCTTTCCCCCACCCCAGGTCTCCTCTTGGCTCTGCCAGGAGCCGGA |
| | | GCCCTGCCACCCTGGCTTTGACGCCGAGAGCTACACGTTCACGGTGCCCCGGCGCCACCTGGAGAGAGGCCGCGTCCTGGGC |
| | | AGAGGTGAGGGCGCGCTGCCGGTGTCCCTGGGCGGAGTAGGGAGGGGTTGGAAAGGGGCCGAGAAATTGCACTCCCACACCC |
| | | CTGGGTTGCAATGGGCAAGCTCCCTCCTTGGCTCAAACGACACCCCTTGGAA |
| 436. | EDG1 | CTTTTTTTCTTTCATTTGTTGTTTGGGGAGGAGGGGTTCTTTTTTTTTTTTTTTTTTTTTTTTT |
| | | TGCTTCTGCCCCAGATCTTTCCTGGACAGTGCGTCTCAGCAGTTCAGATCCGGGGGCCCCCAGCTGACAGAGGGCGTGGGGG |
| | | GTTAAGGCATTAACCCCTCCCAGCCTCTTCCTGAAGAAACCACCCAGCCTTGGCGCGGCGCTGGGTGACTTCGCGTAGCAGG |
| | | CAGGGAACTGGCCGCGGCGAGCGGGACTGGCCATTGGAGTGCTCCGCTGCGGAGGGAGGGGACCCCGACTCGAGTAAGTTTG |
| | | CGAGAGCACTACGCAGTCAGTCGGGGGCAGCAGCAAGATGCGAAGCGAGCCGTACAGATCCCGGGCTCTCCGAACGCAACTT |
| | | CGCCCTGCTTGAGCGAGGTAGGGGGCGGCGAATGGCGGGGGTCGCGGGCGCGGGGAGCCCGGTTGTGGTAGGGCTTAGGTGT |
| | | CAGAAACGAATTTTTAAGTCGAACGAGGCAAACAAAAACTTAGAAAACATGCATCTCCGGGCGCTGAAAATTTGCATCTCTG |
| | | GGCTACTAAGGAGCCCATCTCGATCAGATTGTTCTTGGGCACTTGTGATTTATGGAGTTCAGAAAAAAGGTTAATTCCCAAC |
| | | TGTGGAACCTACGGGTGTAGGGGCAGTGAAAATTGCAGTTGGTCGGAGGAATAGGAGGGAA |
| 437. | EML4 | TCTTTAGTGTCCTTGTAGGACCCACGTGGTGGTCATCTCCCGTCACGGGGGTCCAGTCTTGGCT |
| | | CTGCGGGCTTCCCAGGGGCCACATAGGGAGAGGATCCTTCATGCATCAGAGTATCGCAGGCCAGAGTCTATGCCTTATCCCC |
| | | ACCTGTTTGGAACCAGCTTCCAAACCGTGGTCCCCAGCCTTCAGCAGGCCTACCGCGCAGGCGCAACCACCAGCCTGGCGCG |
| | | AGGCGGACAAACGCCGAGGCCGAGGCAAGGTGAGAGGGCGGGGCTGGCAAGACCCCGCCTCCGTGGCGTCACGTGGGAGGCG |
| | | GAGTCGGAAGTGTGACGGCGTGCGCGGGGCGGGGCTACTGCTGCGCGCGGGCGCGGGGGCGGGGCGGGGCGCGGCGCGGCGC |
| | | GGCGCTCGCGGCTGCTGCCTGGGAGGGAGGCCGGGCAGGCGGCTGAGCGGCGCGGCTCTCAACGTGACGGGGAAGTGGTTCG |
| | | GGCGGCCGCGGCTTACTACCCCAGGGCGAACGGACGGACGACGGAGGCGGGAGCCGGTAGCCGAGCCGGGCGACCTAGAGAA |
| | | CGAGCGGGTCAGGCTCAGCGTCGGCCACTCTGTCGGTCCGCTGAATGAAGTGCCCGCCCCTCTAAGCCCGGAGCCCGGCGCT |
| | | TTCCCCGCAAGATGGACGGTTTCGCCGGCAGTCTCGGTGAGTACGGCTGGGGAGTCCTGCGTCTTCTGCGAAGGGTAGGAAC |
| | | TTTTTTCCTTCCGCACACAGCCCAGGCCCTGCCCTCCCGCCTGCCCCTCGGGGTGGCAGCGGCTCCACTGCACATGTTCCCT |
| | | TCGAGGCTGCCGCCCCTCCGCGGACTCCGGTGGACTGAGGGCCCCTCCCCCATGTCCAACTCTCCTCTGTGTCAGACCCTCC |
| | | TTTCCTCCCGGAGCCCCTTTCAGTGCGGGGCCCTCGTTCCCCCAAAGTGGGAACCCCTTCCTTCTCAGGCCCCCCGATAGCT |
| | | GGCACACCCCTCCCCGTACACTCACGGCTCCCTCCACTTACCCCCCTTCAGAGTTGGGACATTTC |
| 438. | EMR1 | GGGACATGGCCCTGCCCTGGAGCTTGGAGGGACATGGCCTGCCCTTGGAGTCTGAGGTCCCACC |
| | | CTTGGAGTCTTGGGGGGACAAAGCCCTGCGCTGGGGTCTGCGGGGACCCTGCTGTGATCTGGGAGAGGTCCAAGGGATCCCT |
| | | GACCTCACAACCCACAGCGACACGGTGGAGGAGGATGAGGACCTGTATGACTGCGTGGAGAATGAGGAGGCGGAAGGCGACG |
| | | AGATCTATGAGGACCTCATGCGCTCGGAGCCCGTGTCCATGCCGGTGCGTGACGTGGAGGGTCGGGCCTGGGGAGGGCGTGG |
| | | GCGGGGGGCAGCCCCAGGCCCCCCAACACCGGCCTCTCCCCTCGCTCTCAGCCCAAGATGACAGAGTATGACAAGCGCTGCT |
| | | GCTGCCTGCGGGAGATCCAGCAGACGGAGGAGAAGTACACTGACACGCTGGGCTCCATCCAGCAGGTGGGCGCCTCCCACCC |
| | | AGCGCCTGCCGGGCGCATGCGCGGGAGCTGGGCCGGCAGGTGCACGTCCACCTGTCCGGCCGCTCTGGGCAGCTGAGGATTT |
| | | CCTGCTCCCATCGCTTTTCCTTTCTGTGACTGTCTCCGTCTCTGAGTTTCTCTGACTTACATATGTATATATAAATATGAGT |
| | | CTGACGTATATATATATTAAAAAATATATATAAAATATAGGACACTGCCTGCAGGAGCAGTCAAAAAACCAAAAACAAACAA |
| | | AATAAATACAAAATCA |
| 439. | ERalpha | GCAAGGCAACAGTCCCTGGCCGTCCTCCAGCACCTTTGTAATGCATATGAGCTCGGGAGACCAG |
| | | TACTTAAAGTTGGAGGCCCGGGAGCCCAGGAGCTGGCGGAGGGCGTTCGTCCTGGGACTGCACTTGCTCCCGTCGGGTCGCC |
| | | CGGCTTCACCGGACCCGCAGGCTCCCGGGGCAGGGCCGGGGCCAGAGCTCGCGTGTCGGCGGGACATGCGCTGCGTCGCCTC |
| | | TAACCTCGGGCTGTGCTCTTTTTCCAGGTGGCCCGCCGGTTTCTGAGCCTTCTGCCCTGCGGGGACACGGTCTGCACCCTGC |
| | | CCGCGGCCACGGACCATGACCATGACCCTCCACACCAAAGCATCTGGGATGGCCCTACTGCATCAGATCCAAGGGAACGAGC |
| | | TGGAGCCCCTGAACCGTCCGCAGCTCAAGATCCCCCTGGAGCGGCCCCTGGGCGAGGTGTACCTGGACAGCAGCAAGCCCGC |
| | | CGTGTACAACTACCCCGAGGGCGCCGCCTACGAGTTCAACGCCGCGGCCGCCGCCAACGCGCAGGTCTACGGTCAGACCGGC |
| | | CTCCCCTACGGCCCCGGGTCTGAGGCTGCGGCGTTCGGCTCCAACGGCCTGGGGGGTTTCCCCCCACTCAACAGCGTGTCTC |
| | | CGAGCCCGCTGATGCTACTGCACCCGCCGCCGCAGCTGTCGCCTTTCCTGCAGCCCCACGGCCAGCAGGTGCCCTACTACCT |
| | | GGAGAACGAGCCCAGCGGCTACACGGTGCGCGAGGCCGGCCCGCCGGCATTCTACAGGTACCCGCGCCCGCGCCGCCCGTCG |
| | | GGGTGGCCGCCGCGCCCGGCAGGAGGGAGGGAGGGAGGGAGGGAGAAGGGAGAGCCTAGGGAGCTGCGGGAGCCGCGGGACG |
| | | CGCGACCCGAGGGTGCGCGCAGGGAGCCCGGGGCGCGCGGCCCAGCCCGGGGGTTCTGCGTGCAGCCCGCGCTGCGTTCAGA |
| | | GTCAAGTTCTCTCGCCGGGCAGCTGAAAAAAACGTACTCTCCACCCACTTACCGTCCGTGCGAGAGGCAGACCCGAAAGCCC |
| | | GGGCTTCCTAACAAAACACACGTTGGAAAACCAGACAAAGCAGCAGTTATTTGTGGGGGAAAACACCTCCAGGCAAATAAAC |
| | | ACGGGGCGCTTTGAGTCAC |
| 440. | ESR1 | GCAAGGCAACAGTCCCTGGCCGTCCTCCAGCACCTTTGTAATGCATATGAGCTCGGGAGACCAG |
| | | TACTTAAAGTTGGAGGCCCGGGAGCCCAGGAGCTGGCGGAGGGCGTTCGTCCTGGGACTGCACTTGCTCCCGTCGGGTCGCC |
| | | CGGCTTCACCGGACCCGCAGGCTCCCGGGGCAGGGCCGGGGCCAGAGCTCGCGTGTCGGCGGGACATGCGCTGCGTCGCCTC |
| | | TAACCTCGGGCTGTGCTCTTTTTCCAGGTGGCCCGCCGGTTTCTGAGCCTTCTGCCCTGCGGGGACACGGTCTGCACCCTGC |
| | | CCGCGGCCACGGACCATGACCATGACCCTCCACACCAAAGCATCTGGGATGGCCCTACTGCATCAGATCCAAGGGAACGAGC |
| | | TGGAGCCCCTGAACCGTCCGCAGCTCAAGATCCCCCTGGAGCGGCCCCTGGGCGAGGTGTACCTGGACAGCAGCAAGCCCGC |
| | | CGTGTACAACTACCCCGAGGGCGCCGCCTACGAGTTCAACGCCGCGGCCGCCGCCAACGCGCAGGTCTACGGTCAGACCGGC |
| | | CTCCCCTACGGCCCCGGGTCTGAGGCTGCGGCGTTCGGCTCCAACGGCCTGGGGGGTTTCCCCCCACTCAACAGCGTGTCTC |
| | | CGAGCCCGCTGATGCTACTGCACCCGCCGCCGCAGCTGTCGCCTTTCCTGCAGCCCCACGGCCAGCAGGTGCCCTACTACCT |
| | | GGAGAACGAGCCCAGCGGCTACACGGTGCGCGAGGCCGGCCCGCCGGCATTCTACAGGTACCCGCGCCCGCGCCGCCCGTCG |
| | | GGGTGGCCGCCGCGCCCGGCAGGAGGGAGGGAGGGAGGGAGGGAGAAGGGAGAGCCTAGGGAGCTGCGGGAGCCGCGGGACG |
| | | CGCGACCCGAGGGTGCGCGCAGGGAGCCCGGGGCGCGCGGCCCAGCCCGGGGGTTCTGCGTGCAGCCCGCGCTGCGTTCAGA |
| | | GTCAAGTTCTCTCGCCGGGCAGCTGAAAAAAACGTACTCTCCACCCACTTACCGTCCGTGCGAGAGGCAGACCCGAAAGCCC |
| | | GGGCTTCCTAACAAAACACACGTTGGAAAACCAGACAAAGCAGCAGTTATTTGTGGGGGAAAACACCTCCAGGCAAATAAAC |
| | | ACGGGGCGCTTTGAGTCAC |
| 441. | ETS1 | AGGGCCACCAACAGTCCTCCTCCTCCTCCTCCTCCTCCTTCTCCTCCTCGTCCTCCAGATCCAG |
| | | CTGCCAACAGCATCCCCCGCTCCTGAAGAAATGCACCGCCCAGAAGGGAACGGCGAAAGGGGGAAGAAGTCCAGGGGACCCC |
| | | CGGCCTCTGGCCGAGAGCTTGGGTGGGGGCCTCGGCCGTCGCCACTCACCCGGGGAGGGGAAAAGCTCCAGATCGACTTTTT |
| | | CCGTCTTGATGATGGTGAGAGTCGGCTTGAGATCGACGGCCGCCTTCATGGTGCCAGGAGTGGGGGACGTACGGGATGGTAG |
| | | CAAGTTTGCAGTTACTGTTGTTTTTCTTTTTAATGAGGATTAGTAACAGGGGGAAGGGGACGGGGGAAATCCGACTTTCTTC |
| | | CCAAAAATCTCAAATTCCCGCTGCCTTTCTTTCCCCCGCGCCCGGACGGTGCGCGCCCGGCACTCCAGGGGAAGTTGGCACT |
| | | TTGCGGCGAAGTGAGCGCGCTCGGGTCCCAGCCTCGCCCGCGCCGCGCCCGCTCCTCCTGCCCGGCCCTCGCCCGCTCCCTC |
| | | CTCTCCGCCGGCGGCTGCCTCGTTCGCTCTCGATCTCCCGGCCGCTCTCCCTCCCTCTCGCCCTCCCGCGCTCTCCCCTCCT |
| | | CTTTAGGGCGTTTCTCGCGGCGCCGCGTCTCGGCCGCTGGGTCCGCGCGCCCTGGGCCGGGCGATGTCCGCTTGGGGGAGCG |
| | | AGGGGCGGGGCGTCGGGGCAGGGCGGGGAGCCGGGGGCGGGGCCGAGCACCGCGGCCAATCTCCGCCCGCGGCCCAAAGCGA |
| | | AAGGAAGGGCTGGCGAGCGCGGGGCCGGGCCGCGACCGCGGAGGAGCAGTGCGTGGAGCCCCGGGCCTCGGAGCCCAGGCAG |
| | | GGGCGGGAAGGGCCGGGAGCGGGTGTGCAGGCTGGGTGCACAGGCCGCCTCCACCGTTTCGGGAAAGTCCGTCTGATTCTCC |
| | | ACGCATTCTTGAGGGCTTGGTCACCCGCTCTCGCCCCCTCCCAGTAGCAGCATAGTTTTCCAAGTGCGTGTACATCATTCAT |
| | | TCACAAAGACTGACACACAGGACGTGAGGCATGTGGATGAAGGCGAGCGTG |
| 442. | EVI1 | GCTTAAAAAAAAACCGTTTAGGTAGACTTTAGAGAAAGGCCCTTTCAAAAACCAACAGCAAAGG |
| | | AAAAAAAAAAAATCCCCACAATCTAGCCAAAGGGTCCGAATGTGACTTTCTCGCGAAGCAGCACACGATGTTGGACAGCAAA |
| | | GCTGTTACTTGGCAAGGTGGAACTCGGCCGAGAGCGGCTCCAAAGTCGCGTGGCCAGTGCCAGGAATTCAGATTTTGGCAAC |
| | | CGCACCTTGTGCGTCCCCGAAACCGACGGACAGAGACACACGGAGGGAGGGGAAGGAGGAGAAGAGCGCAAGGAAAAGAGGC |
| | | CGACAAGCCGGCAGAGAAACCCACCGAAGGCCGGACGACCCACCCCGGAGACGTGTCCAGACCGCACCGTTTCGCAGGAGGA |
| | | ACAAGGAAATCGCCCGGCTTAGCAACGTAGATAAGCAGCAGGGCTCCGCGAGACTGCGGGCGAGGAGGAAAGAAGGCTGGGG |
| | | GCGGGGGGCGCCCGTAGAAACGGTGCCCCGGCAGGAAACTGTCCGAAGTGACAAACTTTCACATCGCCCAGACTTTTTTTCC |
| | | TTTTAAAGTGAGGAGTTCTCTTACCTTTAGATTTCTATGGCCTCAACTCAGCAATAATAG |
| 443. | FARP1 | GTATTACATCTCTGCTCCTCCCCAGGAGCTGCCCACAAGCACCCCCTACTTCCAAGACCCCTGT |
| | | TCTTGTCTCAACCCGCCCTTCTATCAGGGTCCCTACACTCCACTTCTGCCCAGCTCCTGTCCAGCTGTGAGTTCACTTGAGG |
| | | CCTCTCCCGTGAAATCTGCCCTCTCGGTCTGAGGTCAGGGAACTCCTGTAAGACGCAAAAGGCCCACGGTTCTGAGCCAGTG |
| | | GCCTCCGAAGGCCGGAATCCGCTAGCCCCGGGTCTGGCAGAACGTCACTTGCTGCGCCCTAGGCCTCTTCCCGCCACGCTTA |
| | | GAGAGAGGGACTGGAGGCTCAGAAGGGGGAAGAGAAGGTAACGCGGCAGCTGCTGGGTGGGGACGGTGACTGTGCCCCTAGA |
| | | TCGCTGCCTCCTGGGTCCTGAATCTTGCTCCCCGCTCCGTAGAGTTCCCGAAACCCCGAAGGCTCTCAAGCACCGCAAGCCA |
| | | CTCCTAACCTCCCCCACGCCATTCCCCAAGGGCACTGTCCCCCGAGGCTGGGCGGGCGGGAACTGTCATGGGATTCGCTCGG |
| | | GCGGAGGGCGCTGGGCCAGGACGTGGGCGGGCGCGGGGTGGCCGCCGCTCCCCCAAGCTTAAGTCCCGCGCAGCCCGGGGTG |
| | | GTGCCGGAAGCCCGTGCCTGTGGCTCCGGGGGAGGGGAGGGCTCGGTGGGTCCCTGAAGACTGTCCTCCCCGCGCGACGTCG |
| | | CAGGCGAAGCAGCCCCAGGCAGGACCTCAAAAAGCGACCTGCAGAGTCCGAGCCCCTGACCGCCCGCCTGCCGCTCCCGGCT |
| | | TCGCCGTGCACACGTCTCCCGCTCTCCCCGGTTCGTTTTTCGCCACAGCTGCTGGATTCGCGTCTCGCCAGTGGCGCTCCCC |
| | | GCGCTGCATTAGGTAGCGCCGCGCGGAGCCCGGCCCTGGCCGGAAGCCCAAGAGAGGCGCAGATCCCCGGCGGCGCGCTGGG |
| | | CTCGGGAGGCCCCTGGGCGCACCTGCCCGGGCGGAGCGGAGCGGGGCGGGGCGGGCGCAGGGCGCGGGCCCGGACGCTGGAG |
| | | GGGGCTGTGGCTCTCCCGCGTCCCCGCTGCTCGGGCTGCGCGGCGCCCGCCTCCCCCCGCCGAGGGGCCGGCCCAGACCCGG |
| | | GGAGGGGCGGTGGCCACTGCACTTCCCGCTCGCCGGCCTCAGAGGCGGCGGGTCCGGCGCGGGCGCAGCGGTGCGGGCGCTC |
| | | GGCTGGGGCGCGGGGCGGGGACGCGGCCGCTGCCCGCTTTGCGCCGCTCCTCCCTGCGCGAGTAGCGCTGGCCCCGGCGTCG |
| | | AGGCGGCCATGGCGACCCGGAGCCCGCTCCCCACCCACCCCGCCTGCTCCGCCCTCCCCTCCGCCCCGCGCCACCTTTGATG |
| | | GCTCGGACCTCAGCCGGCCACCGCCAGCCCTGCTCGCGCGCCCGCGCCGCCGCCGCCCGCGGGTATTAATAGCCGGCGCCGC |
| | | CGCGCCCTCGGCCGCCGGGGGCTTGGGAGCCGCCGATCCCGGAGCCCGAGCCGGGAGAGGGAGCCGCCGCAGCCGCCGGCGC |
| | | TGTGGAGGTAGGAGGCGCGCGGTGAACAATGACCGCGGCGGGAGGGCGGGGGCCGGCGGGGTCCGGGCCGCGGGCGGCAACT |
| | | TGTGCGAGTCCAGGCTCCCGCAGCGCACGGCCGCGGCTGCGGGCGAAGGTGGGCGCGTGGTCCCCGAGGTCCTGCCCTGCGC |
| | | AGTCGGGCGGCGGGTCGGGCCCGGGCAGCCCCGGCCACCATCGCAGGAGCTCGGGGGCCTCGGGGCTCCGGGCTGCCCCCTG |
| | | CGCCCCTCTCCCCTCACCTCCGCCGACGTCGGGCTGCGGGGCTCCGCGCCGGTCCCCGCTCGCCTCCCCCGACCCCGGCGCC |
| | | CTTCCCCCGTTTCCTTCCGTCCTACCCGCCCGCTGACAGCGCTGGACGCCGCTTCCGGACCTCGGGCCGCAATCTCGGCCCC |
| | | TGAGGCCGGTTGCGGGCCGGGGAGGTGGCCGCTGGCGCGGATGCCGCCGGGTGCCCGCCGCTCGCCCACGCGCGGCGCGAGG |
| | | GTTCCCGGCGGGTGACAAAGAGGAACACACCCTCTGCCCAAGTTAGATTTGTGTCTCTCTTTACTGTCTGCCTTTATGCAGG |
| | | GCAAAGTTTTTTTTATTTTTTATTTTTTAAAGCAAAGGACAGATTGCTTTGCGGCGAGTATTTCCAAACAACTTCTGATTGT |
| | | GGTTTTACGATTCAAGTTCCGGAAACTTTAGCTGTGTGATCCGGCT |
| 444. | FGFR1 | CACGTCCTCCGCAGTTCATCTCCCACCCCATCCGACCCCAACCCGCACCCCAGGCTTTCCCGGG |
| | | GGTGGAAAAGGACTTAGGAATGCCCCGGATTTTTAGCGTTGAGCCCAAAGCTACCTGCTGAATTGGGGGTGAATCCCCAAAA |
| | | GGGGACCAAAAAGACATGGAGTGGAATGCAACACACACACATAACACACACAACGCATCCCACCTCGCCGCCCACTCCCGCC |
| | | CTCCTCCCCAGTCCAGGAAACCGCGGGCCTCGCCGCCTTCGCCCCGGGCCTGTCCCTCCCGGGACTCACTGAGGAGCCGCCG |
| | | CCGCCTCGCCAGCTCCCGAGCGCGAGTTGGAGGAAAAGTTGGGCGGCGGGAAGAGCGGCGGGACGAGCGCAGGGAGGGGGCG |
| | | CAGGAGACGCGGACGGAGGGAAGGGAGGGGAGACCCAAGGGGCGCGGATCGCCCGGGAGGGAGCCAGGAGGTGAAAGGGGCG |
| | | GGCGGCGAGCGGAGGGAGGCGCCGGCCCCGGCTGGGCTGCGGCGGGCAAAGCGCGCAGCCGGGAGGCTCCGGCGCCGGGGGC |
| | | CGCTCGGGACGCCAAGCCCGCCCCAGATCCGGGGGCGCCGCGGCTCTTACCTCGCTCGGCGTGGAGGTTCCGCCTCGGGAGA |
| | | GTCCGCCGTGGCTTGTGCGAGCGGGCGTGTGCCCGCGTCCCCGGCTCCGGCCCGCCGCCCCCGGGCTCTGTTCGGGTCCTGG |
| | | CGGGGTCGCAAGAGCTCCGCGGCCGGCGCTCGACTCCCGGCGGCGCTCGGTGCTCGGCGCCTCCAGCCCGGGCGGGAACAAT |
| | | GGAGCCGGAGCTGGTGCCCCGGAGGCGGGGCGGGGGGAGGGCTCCCGTCCGCCACCCGGGGTCTCCAGACCTTGTGCCCCCC |
| | | TCCTCCAGAACGCAGCGGCGGCGCCTCACTTTCCTTGCAGACCGGGCTCCATCGCCCTGCGGAGGCCCCGGCGCCGCGGCGT |
| | | GCCCGACTGCAGCACGGGTACCGTGCGCCCTGCGGGGCGCCCCGAGCTCGGACCGGAGAAAAGTCCTTGGGTTCCGCGGCTT |
| | | TTCAAGCAGCGGCGCGCTCGCGGCCGGGGAAGGCGAGGTCGCCGCAATGCGCTACGAGGGGTCTCGGTCCCGTCCGGACGTG |
| | | GCCGCCCAGCTCCCGGCACACCCGGGTTCCTCCGCGCGCTGCGGCTGCACCGGCGTCCCGGCTCCCGCTAGCTGCCGCCCGC |
| | | CGCCGAGGACGCCGCGCCTGTGGCCGCAAGAGCGCTCCGAGCGCTATGCGCCGGCGGGGCGCGAGGGCTCGCTCTCTCACTC |
| | | CCACCCGCGCTGGTCCCTCCTCCGCCTCCGCCCCCTGCAGCTGCCTGCTGGGCCTTGTAGTTCCCCAGCCGCGGCCCCAAAG |
| | | GACGGGGAGCGGGCCGAGCTGTCCGGCGCTCTGCCCTTCTCTTCCTACAGCCTGGTCTCCTTTGGCGTTTGCGCCCCTGCAT |
| | | CTGAGCACGTCCCAGAGGGCGCCGGAATGTCAGGCCCTAGACATAGCAGCGGTTTGGGGGCTAGGGGGCAGGTGAAGAGACG |
| | | CCGCAGCCTTGAGGTTGTACCTGGTAGCGTGGGCTTTGGAGTCTGTGCGCCTTGGGGAAATGATTTTCGCGCTCTAAATCTC |
| | | TGTTTTCCCATCTGTAAATGGGGGATAATAGTGCTTTTCACATAGAGTTGAGGCGTCAAC |
| 445. | FHL2 | AACACTCAATATTTTCTTCCCTCTATTTCTGGTATGCTAAGATATCACACGAGTGCGACTTAAG |
| | | AAAAACTTAGAATCTTTTCATTTAAAATAAGCCATTTTTGTTTTAACCGAATGGTTCAGTTATTAAATGTCTACCTGAGTCA |
| | | AGGAGCGAGGTCACACTGCAAACTCCACTTATTTCTACTAAAGGGAAATGGCCCGGACTCCCCCGCGCAGACCACCGTGCCA |
| | | GGACAGCCCGCTCGGGAGTCGGGCCTGGAAGCAGGCGGACAGCGTCACCTCCCCGCAGCCGCCGGCTGGGACCCGCGGCCAG |
| | | CCTTTACCCAGGCTCGCCCGGTCCCTGCCCGCATGGCGGTGCCCCACCTGGTGAGGACGCGGGCTGCAGTCCTCCCTCTGGG |
| | | GATGAGTCGGTGGGCTCCTCTGAGCTCCAGCGCCCCCAGGGTCTAGACCCCTCCCATTGCTCCGCTCCCCTCTCCCCGTGGT |
| | | CTCCCCACCCCCAACCCCCAGGGTTCGGCTCTCCTCTCCCGGGTCTACCCCACAGCTCAACTCCTTTCTCCCGGCTTCTCCC |
| | | CGCACGGTTCGGGTCCCCTCTCCCAGTGGTCTTCCCGGACCCACAGCTCTGCTCTCCTCTCACCAGTCTCCCCAACTCCGGC |
| | | TCTGCTCCCCTCTCCTTGGGTCTCGCACCGGATTCGGCCCCCACTTCCGAGCCCTGGTGGCTAAGCCCCTCGGCCTCCCTCC |
| | | GGGGCGCAGGGGGTTGGAGGTACTCACGGCACCGCAGCGGGCCGGGGACTCCCGGACGGGGCTGGAGGGCGCGGGCGGCTGG |
| | | TGGCTGCGGCTCCGCTGCCGGCCGAGTGGAGCGCTGCGCAGCTCCCGCCCTCGAGAAACCCCGCCGTGTCATTTGACCATAT |
| | | AAGGAGATGCACGCCTCGGCTCGCTGGCACCGGGCGTGGGGCGCGGGGGGCGGGCGCCCCCAGGCCTCGCGGTTGCCGTGCC |
| | | CCCGCCCCGGGCTGCGGCGGTCCCGGCCCGTACCCTTTGTTTGCCAGGGCTCCTTTCTTCGTGCCCTCCGGGTCTTGGGAGC |
| | | ACAGTAGTTATCGGGAGCGTCGCCTCCGGCGTGGGCTCTCGGGCGCGAGTTTCGGACGAGGCCTGGGCGCGGTGGCAGGGGT |
| | | CTGCCCACGCCGGGATCTCTGCCTGGTCCCAGGAGCGGGAGACTGGAGAAGCCCCAGGACGTGCCGGGGGAGGCGGAGGGAG |
| | | GAGGGGGTCACTTCTCAGGAGGCATGTGCCTGGGATATATTTGCAAGGTGGACGTTTTTCCTTCTTACCCTGCAGATGCTTC |
| | | TTTACGAAATGAACACCACAGATGACCATAAAGAATCCTTCGTTCCACTACGGAGGGGACTAAAGGAAGGTCCTATCCCCAC |
| | | CTCTCCCTCTCGGGGCCAGAAGACTAGTT |
| 446. | FLI1 | CCTTTTGAAATTTTTTCTTCCAATTGACTTGGATCTCCTCTGGATTTTTTTTCCCTCTTTCCCC |
| | | CGTCGGACTCTTAAATAAAAGTGAGCAAGTCCAAAGCGTGGTCCGGAGAGCGTGGACAAGGTAACCTGTTTTGGATGTCCGG |
| | | GAGAAGGGAGTCCGGCCGAAAGGGCCCAGCCCCTCGAGGCTGCTCCAACTGGGGCGCGCGGCGGCCCAGGAGGGAGGCGGGG |
| | | AGGTCTCCGGGTTGGGCCGGGGAACGCCGGCCTCCAGGCTGGGTAGGGAGGACGGTCCAGAGCGCGGGCCTCCGGGTCTCAC |
| | | CCCGGGAAAAGTCTGGGTCTGCTCTGCGCTGTTGCGTGCTGCGCTCCTGGGACCTGCTGACTGTCCTAGAGGGAACGTCCCG |
| | | CGGTAGTGTCAGTTCCCTGGGGACCAGGAAGGGCGACTCTTGCCTGGGAGTCAGTGCTTGGGCCGTCGGGTCCGGGTAAGCG |
| | | CTGGGGGTTTGGCTCGAGAAGATGCGGATAGGTGGCCTAGGGCGCCGCGGATCTCCGCCCTCTGCGGGCACCGGGAGCCCAG |
| | | GGTCGAGGCGCGCCGCCCCGAGGGGCCCCTTCTCCTAACAATGCGCCCGGAATGGAGTCCGCGCCCTGACAGCGCGGGTCAG |
| | | CCCGAAACCGCCCAGAATCCGCTTCGATGAGTGGGTGAGCCGCTCTGGGAGTCTGCCGCGTCTCGGCAGCGGAGGGCCTGAG |
| | | TGGAAAAATTCTTATTGGATGCACTTTTTAATGGTTGGTTTGCCTCGGTTTTCGTCCGAGTCTTCCCCGGCAGTAGGCGCTG |
| | | GGTTACCCGCAGCCCTAGCCAACTCTCCCTCCATACCCCCCCTACCCCACCCCCAAAAAGTTTGTCTCCGGCTGGAAAAAGC |
| | | ATTAATGAACGTATCTGTGGGAAGAAACGGAAAGTGAATGAGTCAGGAGGGCCACCTTCGGCCAACCGCCTCAGGAAAAGCA |
| | | AAGAAGCCAAAGGTCTTTGGCTTTGGATTTTGGGGGAAGGTGATGGGGGGAGGTTCAGACTTCGGGAATCAGGGCGCGGACG |
| | | CTGGGCGTGGACCCCGTCATTGTTCCCGGCTAGCTCTTATCTCCCAGGAGCAAGTATCCTGTGTGCGCAGCGCATGAATGTG |
| | | TCTGGGCATCTCCGCGTATATTTATATAGTGTGTGATGCGAAAAGCAGGACCAGCAGGGGAGGAAGAGGGGGTGTGGGGGGG |
| | | GAGGGAAGACGAGGGAGAGCAGAGGGGAGAGAAGAGAGAGGAGAGCTCGAGGCGAGAGAGAGAGAGAGAGAGAGAGAGAGAG |
| | | AGAGAGAGAGAGAGAGAGAGAGATAGGACTTCCTCCCCGATTCGCAAAGTGAAGTCACTTCCCAAAATTAGCTGAAAAAAAA |
| | | GTTTCATCCGGTTAACTGTCTCTTTCGCTCCGCTACAACAACAAACGTGCACAGGGGAGTGAGGGCAGGGCGCTCGCAGGGG |
| | | GCACGCAGGGAGGGCCCAGGGCGCCAGGGAGGCCGCGCCGGGCTAATCCGAAGGGGCTGCGAGGTCAGGCTGTAACCGGGTC |
| | | AATGTGTGGAATATTGGGGGGCTCGGCTGCAGACTTGGCCAAATGGACGGGACTATTAAGGTAAGCGGCGGGGCAACGGACG |
| | | CGGGCGGCGGGGACCGGCCGGGGAGGCGAAGCGGCGGGCGGGTAGGTGCGGGGCCCGCGTCCCGGAAGACGTGGCCTCTCTC |
| | | CCTTCCCTCCGCGCCCCGGCTTCGCGCCGGCTCCTCCGGCGCTCGGGTGGCGAGTCCTACTCGCGGGCCAGCCGGCCAGGCG |
| | | CCCGCATTGAGGGCGACCCTCCCCCGAAATCCCAGCCCCCAAAGTGGAGCCCCGGCCCCCCTCCCACCTCGCTGCCCGCCGG |
| | | GGCTGCAGGAGGGGACGGCGGGAAACCGGGGGACCCCAGGGTACGGAACGGAGTCAAGGGCGAGAGACCTCGGGGTCCACCA |
| | | CTCCCACCGACACCCGGGGCTCCCGTGGCCCCAGCGCTAGGCACTGGGCTTCCTCTCCGCAGAGGCGGAACACGGCGGGGGC |
| | | GGGGGCTGCAGTCGCCCGGGCTCGGGTCCCTGCCTGGTGGGCCCTGGGGTGGGTAAGTGGCCTCGTTGCACGAGGGTAGGGG |
| | | TGCAAGTGAGTGTGTCGGGGGATCTATCTGAGCGGGCTCCTCCAGATGGAAGCCCCTGTTTACATGTCAGCGCTTTCCCTTC |
| | | CTCTCGGCACTCAGTCGCCCGGCTCTAGGCGCTGGAGAGCCGCCGGCTCCGCGGGACTCCTGGGCCGGCCTCGCCGCCTCTC |
| | | CGGCGGGGAACCTTCCCCAGCCCCCGTCCGCACAGATCCCTAGCGCCCCGAGCCCCCGCCCTTCGCGCCTAGGCGTGCGCCG |
| | | GCTCCAGGACCAGGGCTCCTGGAGCTGTCGCCTCCGAAAGGGTCCTGCGTCCTTCGGAGCCGCCTCCGTTGCAGGGGCCGGC |
| | | TGTGAGCCCGCGCCCCGCGCCCGCCCGGCCATTCCCACCCCAGCCGCCGCCGCCAAGCCTGGGTCTAGGTTGCAAAGTGCAA |
| | | CCCAGGCGGAAAGGTG |
| 447. | FLJ21820 | GTTAAGGTCTTCAGCGTGAATAAAAGGCGGCGTGAGCACTAAACGCCGGGGGCAAAGAACACTC |
| | | CCGGCCCAGAGTTTGACCCCTCCTAGCGAGTTCTTCCCGGCCAGCTGCCCAACTTTTTGGCCTCTACTCCTCCGCGGGGGCA |
| | | CCATAAGACTCCCTACAGGTGGGCTACACTTAGGGGTTCAAATGCTGCATTACCGCGCTCGGGTGAGCGACTGCCCGGCCTG |
| | | CAACTCAAGGGACTCCCAGGCCAAGTGGCTCCGAACTCGCGAGCTGTCGGAACAGTATTATTTTTGCTGCTACGGGCCATCT |
| | | GGCCCAGGGCAACAGGGGCTCTGCGAGGAACCATAGAGAAGAAGGGGCGCCAGATCCCGCGTGCCTGCCCACCCGTCCTTCC |
| | | TTTGGGCAGTCACCCTGAGCTCGGCAAGCTGTACCTCACCTGTCCACCTGGAAGGCTGCCCGCTCTCCCTGAGGGTCCTGGG |
| | | AAGGCGGCACGAGACCGGAAGTGCCCCCCTCAGGTGACGTCACAGTACCGCGCCGGAGCTGATCCGGGAAGCCGATGTCAGC |
| | | CTGGCGCGCGGCGCACAGCTGGCTGGGAGATTTCTTACTGGCTTGGAGCCGGTGGTCCAGATCAGAGAGACCTCTTGGAGGG |
| | | TGTCGGGGTTTTCCTTTCTTTTTCATTTGAATTTTATCCGCCTTTTATTTTCTGTCATCCTTGATGTGCTTCTGGCTTCTGG |
| | | CTTCCCTTTCCTTCTCTCTCATTTGTTCCATGTCTGCCCCTCTTTTCTC |
| 448. | FLJ23058 | GCCAGCCTGAGACAGAAATTGCACTCAGCACACCCTCTGCTCCTCTCTGAGAAGAAGCCATTTT |
| | | CCAGACCATATGACACGTCCATCTTTCAGGGAAATTAGTATTTCTCTGACTCCGACAGCCCCCAGAGGGTCCCCGGCAGGTT |
| | | CCTCGGCCTCTGGACAGACAGGGCTGCCTGGACCTGGTCCCTGGCAGGTTCCTCGGCCCCGTCTTCCGGCCACACAGAAGTC |
| | | CATCCGGTGACCCTTACAGAGCATGGGTGGGCGGGTGGTCTTTCCTGGCTGGGCAGGGGCGCACTGGCCAAGGCCGGCCACT |
| | | GATGCCCCGCGCGCCTTGCCCCCAGAATGCGCTGTACCAGTCCTGCCACGAGGATGAGAACGACGTGCAGACCATCTCCCAC |
| | | AAGTGCCAGGTCGTGGCGCGCGAGCAGTATGAGCAGATGGCCCGGAGCCGCAAGTGCCAGGACCGGCAGGACCTCTACTACC |
| | | TGGCGGGCACCTACGACCCCACCACCGGGCGCCTGGTGACGGCTGATGGCGTGCCCATCCTATGCTGAGCCGCCCACCGCAG |
| | | ATGCCTCCCACGTGCGCCAGGGACCCTGTGTGCGGAGCCTGGCGTCGGCCAAGCCACCGGGCAGGAGGCAGCCCCGGCCTCC |
| | | CAAGGGCGCATCTGAGCAAATATGCAAAAGCCCACAGGGCAAGACCCAGGCTTTCTTACGGTTTTCCCTGGAAAGAGCGCTC |
| | | CAGGTGTCGGAATCCAGTCCCGTCCCATCCTCTGCGGAGGGTCGGGCTGTGGCCCTCATGGGTCCCCGGCCCGCCCCACCCA |
| | | CAGCGCCCTCCGTTTCCCGCACCGGCAGTTCACGGGAGATTTGAATCCAAGCCATATTCCCTAGTACCTCCGACTGTCTCCC |
| | | ACCAGGGAAAGCAGAAATCAGGTGTGTTGTCTATTTATTTCTCTATGTAAATATTGTATTTCTGCGGGGAAATTTTATGGTA |
| | | AAAAGTGGAAAAGGGTTTTTCCCCATCCGCGTGACAAGGTGTGTGTGAGCGTGTATGTGTGTGCGCGTGT |
| 449. | FLJ25409 | TCAAAGTGATGTCATTCATGACTCCGAAATGGTTACCATGGCGACTATCAGACATATTAAGCTG |
| | | TCAGGGAAAGAGCCTGTTTAATCCAGTCTTCAATCACGGGTTTCCTCGGGCGTTAGCCACTGCCTGCGGAGTGTGGACTGGC |
| | | AGAAAAACGTCACCTTCGGGCCCCCCAGCACCAACGCGCCTTAAACCAAGCCCCACAAAGAAGTCACTTCGGCGATGTAAAC |
| | | GCGACTTTGCGGCTTCCTGGGCTGCTAAGGTGAAGGACGCCCGCCCTCCCCAGAACTTGAGAGCTTATTGGTTGGCGATGTC |
| | | GTCACTCAGAGTGACGTCAGGGGCGAGGAAAGAGGCGGAACCGTAGAGACTTGGCTTCGGGCCCTTCTAGCTTGGGGGTCCC |
| | | GGGAAGGAGCTGGGAGGACCTAGGCGGCCGTTCCGCGGAGCCCGGCCGAGGAGGTAGGGGTGGGCAGGCCCAGCCCTCAGGC |
| | | CGCGACCTGGTGGGCCGCGCTGCCTTTTTTTCCTCCGGATCCCGCTCGGTCCGCATCCTGCTCCTCTCATCAGAGCATGATC |
| | | CAGGCTTCTGACAGCTTCCAGTTCCCACCGCGCTCTCCTCCACCCACGACCTCATCCCCGCCAGTTCCCCAGCACAGCGCGA |
| | | ACTCTGAAGCTCGGCCTCTCCTCCCCCTTCCCCGCACAGCGCGCTCCGTCCTTTTTTCCGTCCCAGGCCCTGCTTCTTTCTC |
| | | CTCGGCTTCATTTCCAACAACCCCGATCATCCACGAGGGTCGTCGTTCCCGTGCCTGTCCCACCTCCGCACCAGAAAACCAA |
| | | AGCATATCCCCCAGACCCCGCCGCATGCAGGCCGCGCACACATCAGGCGCGCCGAAGCCGCCGGGCGAGGAAGCCGAGGGCG |
| | | CGTTTTGCTCCCGTGTGGTGATGGGCAAAGCCGAGGGAGCCCGGGAGCAGCGGGCGTGGGGCGGCGCCAATGCGAGTGCGAG |
| | | TGGTGTCCGCCGCCCGGGAGCGCCCGGGCCCGAGCGGATTAACCGCCGCCTCAGGTGTCGTCTCCTGCCCGCGAAACACCAC |
| | | CCACCGTTAGTGTGGGTCCACAGGTTTCGAGCCCCTGTTGAAAATGTCAGCTCTGTGGCTAAGGTTTCTATTCAGGAGAGCT |
| | | CCTGGAACTCTTTTCCCAACCACA |
| 450. | FLT3 | CTATTCGTTGATCCAACCAAAATTCGGCGGGGCTGGGGGGTATCGTTATTGAGGATGACTCGCT |
| | | CTAGGGGAGAACGCGGACGCATTTTCCCATCAGAGTTCCCTCCATAAATCAAAACCTCAAAAGAAGAGTTAAAGACGACACC |
| | | CAGGTGTCTACAGTATCCAAGGGCCGGGCCAGGAGAGGTCCTTGGCCACCTGGCGCCGAGTTCGCGGCCGCAGACTCCCACG |
| | | GACGGCCCAGCACCCGAGCGCGCGGGCCGAGCTGCCCCAGACCCGGCGCAGCGCCTCGGCGAGGCCGTCTGCGAAGTCCGGG |
| | | CCGCCACTCGGGACCGCGGCCCGAGCCAGAGGAGAGACTTCGGAGAAGAGGGAAGAGGACGCGGGGTGGGAAACGCCGGGGC |
| | | CGCAGGCAAGTAGTGGGCGAGTCCGGAGGGCGCGAAAGAGGGGAGGGGCGCGGGAGGCAATGGAAGGAGCGAGCGCGGGGAG |
| | | GAGCGAGGCGGCTGGGCCGGAGGAGGCGCGCGCCCGGGTCCACACTGCGGGGTGGGGGCTGAGGGACCGCGAGGGGCTGCGA |
| | | GCGAGCGAGCGGGGCCTTACCGAGCAGCGGCAGCTGGCCGCCGTCGCGCGCCAACGCCGGCATGGCCTCCGGAGCCCGGGGT |
| | | CCCCAGGCCGCGCCGGCCCAGCCCTGCGATGCCGCCTGGAGCGGCGCGCCTCGCGCTGCAGGTGGCTCTCTTAAGGATGCGC |
| | | GTCACCGACCGCAAATTCCCTCGGACTGGTGCAAAGTGGAAGGGGGGAGGAACCCTCTCCCCAGAGGCAGGGACCGAGGCGG |
| | | GAGCGGAGAGGTTGGGCAGAGCCGAGCAGCCGGTGCCTGGCGCAGCGCTGCAGCCGAGCCCACGTGCCCGCCGCGGTCCGCA |
| | | GCCTGTGCTCGGCGGCCCCCGGGGCTCCCCGACTGCGGCGGGGAAGATTGGGCGCGGTCCCGCCGGAGAAGTTGCACTGGCT |
| | | CCGCGGCTCTGCCGCCTTGCTGCCCCTGTGAGTCCCCGAACTCTGTCGTTTGGATCCGGGGCGTGGGGACTGCAGGGAGCCA |
| | | CAAACCACCCTCCTAGTGCGCTGGGATCTTTGAGGCCCTGAGAAAGGCGCTGAATCCGCTCGGCACGCTGGCAACTCCCGCC |
| | | AGCGCACAGGCGAGCATTCCATTAAGAACCATTCCATTCTTAATTGTGGACTGATTTCCTTTGTCTCTGCCTCCATCTCCTA |
| | | GGATGCTCAGGACCCAGTCAGCGGCGGGATCAAGAAAAACAGGCAGGGTTTCAATGTCAACCAAGGCAATTAGGGAGGTTAA |
| | | CAAACAGCCTTCAAAACAAAACACACAGAAAATACATCCCAGAGGATG |
| 451. | FN14 | GTAGCTCGGATTGCAGGCGCGCGCCACCACGCCCGGCTAATTTTTGTATTTCGAGTAGAGACGG |
| | | CGTTTCACCACGTTGGTCAGGCTGCTCTCGAACTCCTGACCTCGTAATCCGCCTGCCTCAGACTCCCAAACTGATGGGATTA |
| | | AAGGCGTGAGCCACCGCGTCCGGCCGTTCGTGTCTGATTTCTATATGTGCTGCCGAAGCGAGCACTCGTGTCTAATTTTTGA |
| | | TTTCCCCACACCACGCACGCTGGACTAGATCACCGGCTGGGCAAGGAAGGGGGTCTGCGTCCCTGCGGGGTCCTGGCAGCTC |
| | | CCGCGCCAGGACTTTGTTGAATGAATGACTGAACTGAGCGGGCGCCGCGGGGCGGGGCGTCCCGAAGCGGACCTCAAGGCGG |
| | | GCGGAGGCGAGAGCTCCGCCCCGGAAGGCGGGGGCGGGGGCGGGGCGGCGGCCGTGGGTCCCTGCCGGCCGGCGGCGGGCGC |
| | | AGACAGCGGCGGGCGCAGGACGTGCACTATGGCTCGGGGCTCGCTGCGCCGGTTGCTGCGGCTCCTCGTGCTGGGGCTCTGG |
| | | CTGGCGTTGCTGCGCTCCGTGGCCGGGGAGCAAGCGCCAGGTACGCGGGACTCCGGGGTCGGGGAACCCCGGGCCGGGGCTC |
| | | GGGAGCCGGACTGGGTGTCTGGAGAACAGCGCCGAACTGGGGGAACAGGCGGATGTGGGGATCTGGGGTCAGGTGGCCTTCA |
| | | AACAGCTCGCTCCCGGCTGTGGGAAGTTCCATCACTCTCCAAGCTTCATTCACAATCCGGGCCTCAGTTTCTCCCACCGTCC |
| | | AATTGGGGTTGGGGGGGGTCTTCAGTTCCACAAGTCCGAAGTACTTCGAATGGAGAGAGAAAGTGAGTGAGGGCGGGATGTT |
| | | TAGTCTAGGAAGGGGCACGCCCCCAGCCTGGTCCGTACAGGGTCTAACTAGCCCCAGTGATCGACGAGTTGGGGAGGCCGTC |
| | | GAGAGGCACTAGGGTCTGTGCCCTCTATACCATGTGGGGTGCCCCCTTCCATCCGGTGACTTCAGTGCCCAGCTATGCGGGG |
| | | GGAGGGACTGGGGTCGGGCCCGGTGCCCAGGAGTGAGTCACCCGCCGGCGGCAGGGGGGTTGTGGGGACCTGACCCCCCCCA |
| | | CCCCCAGCGTCCCGCGCTACGCCCTCCCTCCCGGAGCTGCCGTGTCCCGGTCGGGCCGTGCGGTCACCTGTGAGGAATGCGC |
| | | GGGGAGGGCGCCGGTGACTCACGTTATTCGAGCCGGCCGACCCTGACCTCAGACCCCAGAATAGCCGAGTCCCGCCCCCAGC |
| | | CTCTGACCCGAGGCCCCCTCCCCAGGCACCGCCCCCTGCTCCCGCGGCAGCTCCTGGAGCGCGGACCTGGACAAGTGCATGG |
| | | ACTGCGCGTCTTGCAGGGCGCGACCGCACAGCGACTTCTGCCTGGGCTGTGAGTGGGGGGCAGGGCCAGTGGCCAGCGGACC |
| | | CCAGACTGGGAGGGAGGGTGGCTGGTGCGCAGAGCGGGGCCGAGAGCTTTGCATCTGGGAAATCATTCGGGAGGAGGTGGGA |
| | | GCTGGGAGGGGGCTCCGGTCAGGGAGGAGGCCACGTTTGGGAGAAGGCAGAAGGCTCAGTCTTAGGGGG |
| 452. | FOXO1A | CGGGAGGCTGAGACAGGAGAATCGCTTGAACCAGAGAGTCGGAGGTTGCAGTGAGCCGAGATCG |
| | | CGCCACGGTACTCCAGCCTGGCGACAGAGCGAGACTCCGTCTCGGAGGAGGGGAGGGAGGTGTTTAGCTCAACTAGACAGCC |
| | | TTTACAGCAATTCCGCCACCGGTGACAGTGAGAAACTGTCAGCTTGGGGAGGCCGCGCCACGACTGGACCCCGCCCCCGGGA |
| | | AGCCTCCCCGGAGCCGAGGGCCCCCGGGCCTTCTCCTCGGAGTCGGGAAGTGCCTGCCCCCACGCTAAGTCTCTCCTGGCCA |
| | | CGCTCTGAAAGGCAAAAATGGCAAAAATCCTCCTTCAAGGCACCGCTCCCTCGACGCGCACCGCCACTCCGACTCCCTCAAA |
| | | ACATTGGTCGCAAAGCCCCCCCGCCTGGTCCTCCATGCTCAAGAATAGGGTGGCAAGGCAGGAAAAGACTCACACATCCCAG |
| | | CCCCCGGCCCCTCAGGGCAGCCTCTGTGTTTCTAACTAAAAGCCTCCTACCAGGGGCAAGACCAAGTTCGGGCGCTTCCCCG |
| | | CATGGAGAAACGCTGGCCCCACGGCGCCCGGCCCCGACGCGCTGGCCCTTTAAAGGCGCGGGCTTCCTGCGCCCGGGGCCCC |
| | | TCCCACGCCACCGCCACCGCCACCGCCACCGCCCGCGCCCGCCCCCGCCGAGGGTCACACTTGAACTTTAGCTCGCCCCGCG |
| | | CGGCCAGGCGCCGGCGCTCCGCAGGCGGTGGCCGCCCCTTCGCACGTGTCGGAGGCACCACCTCGGGCTCGGGACGCCGGGC |
| | | CCCCGGCACTCGAGGCCACTCCGGAGAAAACCAAAACAAAGCAAAATGCGCACCGCCTCCAGAAACTGCCCGGCAGGCCCTG |
| | | CACCGTCCCCGGCCGGTCGCGCCCTGCCTGTCCCCTCCACCTGCAGCCCGGTCCCGGGGCAGCGAGGCTCCGGGGCCCCTCG |
| | | CCGCGCCCTCCCCCGCCGCGCCCGGCGGAAACAGCTCCGCGGCGCGGCCGGAGAGAACCCGCCCTCCCCCCGCGGAGGTCCG |
| | | GGAGGGAAGGGGCAGCCGAAGCAGTCGGCGCGGGCCGGGGGTTGCCGCTCCCAGCGAACCCCTTTCTCCTTTCACTGGCAAA |
| | | CTTTTCGGCCTCGCTCTGACGTCCACTTCTTGGCGCACTTTCTTTACTTAGTTCCCCAACGAGCCCCTTACCGCGTCCCACG |
| | | CGAACTCCTGACTGGCGCGCACGCACACCTACTGCCGTCCCCGACCGGACCCGGGCGAGGCCACCGCGACCACCGCTTCTCG |
| | | CCCGCCCTCCTGGGAACGCGCTGCCCTCCTGCTCCGCACCTTCAGGCCGAGCAAACCTGCACAGCTGCGCCCTCGCCTGACC |
| | | CACCGCGCCCCCAAGGTCCGGCCGCGCGCCGAGTCCACTCACCTTCCAGCCCGCCGAGCTGTTGCTGTCACCCTTATCCTTG |
| | | AAGTAGGGCACGCTCTTGACCATCCACTCGTAGATCTGCGACAGCGTGAGCCGCTTCTCCGCCGAGCTCTCGATGGCCTTGG |
| | | TGATGAGGTCGGCGTAGGACAGGTTGCCCCACGCGTTGCGGCGGGACGAGCTGCTCTTGCGCGGCTGCCCCGCGAGCGGCCC |
| | | AGCGGCGGCGGGGGGCACCGGCGGGTGCTGCGACAGCGGCCCGGGCGGCGGGGGCTGCGGTGGCGCTGGGTGCAGGCAGCCC |
| | | GCCTCCGGGCCCTGGAAGTCCCCGCACAGCCCCCCGGTGGCGGCCGCGGCGGCCGCCGCCGCCACCGCCGCCGCCACGGAGC |
| | | CGGGCGCCTGCGGGAAGTCCTCGCTCTCCTCCAGCAAGCTCAGGTTGCTCATGAAGTCGGCGCTGACAGCGGCAGCCGAGGC |
| | | CGAGGGCAGGCCCGCCGCGGCGTCGGGGTTGGCAGCCGCGCTGCCCGACGGCGCCGGGCTGGAGGTGGCCGAGTTGGACTGG |
| | | CTAAACTCCGGCCTGGGCAGCGGCCAGGTGCACGAGCGCGGCCGGGGCAGCGGCTCGAAGTCCGGGTCGATCTCCACCACCT |
| | | GAGGCGCCTCGGCCATGGTGACCCCCGCCCCTCCCCCAGCCGCAGGAGAGCCAAGAGGGGGAGAACGCAGCACTGGGGGCGG |
| | | ACGGGGAGGGGGCGCGAAGGGACGGTCCGAGATTTGGGGGAACGAAGCCGGTGCGGCGAGCGGACGGAAACTGGGAGGAAGG |
| | | CGCGGCGGAGTGGAAGCGCGAGCCCAGAACTTAACTTCGCGGGGCCATCCACATCGAGGCTCCTCGGGGTCCGCCGCACGGA |
| | | CTGGACGGCCGGCCAGAGCCGCCGGGCCGGGGCAGAGCCTGCGCCGCGCTCCAGCTGACAGGGCCGCGGACGGAAGGACGGA |
| | | CGGACGCCGCGGGCCGCTTGCTCTCCCCAGCGGCGCGCCCGCTGCGCTGCTGCCTGTTGAATGTGGCGGCTGCGGCAGCGGC |
| | | TGCTGCGACTACCAGGCCGCCCGACTTACGGGATCTGCCGCCGCCCCCCGCCCGCGGCGGCGCGCGCGCCGGCCCGCCCCTG |
| | | ACCGACAGCCCGCGCGGCCAATGGGCATGCGGCACCGCCGCCCGGGCAGCCAGTGGGCGCCGGGCTGGGTGGGGCCCGGTTT |
| | | TCCACGGGGAGGCGGCGGTGGGCTGGTGGGGGGTAGTGGGGTGTTTTTCTCTTTCACACACTCACCTCCTTTTTTTTTTTTT |
| | | GGATCTCTATTATTTTCTGGTAATTCTCGAGTGTTTCTGTGATTCTCTCGCCTTCTCAGTGTTTTGATTGCTAGGAAGCAAA |
| | | CCAGCGTGGAGGCGCCGGCGACACTTTGTTTACTACGGAGCAGCAGAGCCGAGTACTCGGGAAGCCCGGGTGGGAGGAGGCG |
| | | CTCGCTGCTCCCTGACCTCCGCTGCGGGCCGAGCCCGGCGGGCTGGCAGGGCAGGGGGCCGAGGGCCGGGGGCGCGGGGTGG |
| | | GCGGGCGGAGGCGGCCGCGAGGAATTCTACTCAATCGCTCCCTCCTGGCTCCACCCACGATGTCTTTGCTGAACGACGTGGG |
| | | GAATCGGTGGGTTTTGTTTTGGTTTAATGTTTTCTTTCGCTGCGATCTGTCAAGTCCTCCGGCCCCCTCGCGAGCGGCACAC |
| | | GCCCCCCACCCCCGGCGCCGCGGCTCCTGCAGTCGAGTCCGCGCCGAGGGACCCTTCTCCGTGCCCACCGGTCCGCACCCCC |
| | | GGGCTCAGCCTGTGCCATTCGGTCTAGCCAGAGGCGCGTCACCCAGCCGGCAGCCCCCAGCCGGATTCACTGTATTCTTGAC |
| | | CTTTTTTAAAATGCTAGAAATGAGGAACAGGGGCTCCGGCGCGGGGAGGAGATTATCTGGCCTCCCTGAACACACGAATCCA |
| | | AAGGCACCCGCGACCGCATGCCCTCCGTGGGAACCTCTTCCCCTGGGCGGGAATCTCCCGCTCCGTCCACTAAGTCCAGGGC |
| | | CGGCAGCGAAGGGCTCAGGAGCGCAGTTAGAGAGAACCAGGTCATTTTATTACTTATCCCTAATTAAATTTAGCTGTTTGTT |
| | | ACTTCTTTTTTATTTGCAACGGAAGTGATCGGGTGTTAAGTAAAAACGCAAGGGGGGCGGGAGATAGGACCAAAGCCTTGGG |
| | | GCGCAGCCTGCCCCACCCCCAAATTCCTTGAGGCTACTTGCTGTGTGTGTCAGTCAACC |
| 453. | GAGED2 | GGGACCTGGGAAGGAGCATAGGACAGGGCAAGGCGGGATAAGGAGGGGCACCACAGCCCTTAAG |
| | | GCACGAGGGAACCTCACTGCGCATGCTCCTTTGGTGCCCACCTCAGTGCGCATGTTCACTGGGCGTCTTCCCATCGGCCCCT |
| | | TCGCCAGTGTGGGGAACGCGGCGGAGCTGTGAGCCGGCGACTCGGGTCCCTGAGGTCTGGATTCTTTCTCCGCTACTGAGAC |
| | | ACGGCGGGTAGGTCCACAGGCAGATCCAACTGGGAGTTGAAGTGTGAGTGAGAGTGAAGAGGAACCAGCAGGCTTCCGGAGG |
| | | GTTGTGTGGTCAGTGACTCAGAGTGAGAAGGCCCTCGAAGTCGTCGTCCCTCTCATGCGGTGCCACGCCCATGGACCTTCTT |
| | | GTCTCGTCACGGCCATAACTAGGGAGGAAGGAGGGC |
| 454. | GAS7 | CTCAGCAGGTCCTGTGCAGCCCTGGGGCCAGGCCAGAACCCCAGGAACAAGCCACTTTCGGGTT |
| | | CTGAGCCTTAGCCTCGGGGAGCTGGACCAGGGGAGCAGGCAGTGGCGGGGGGTGCTACGGCACCCCAGGATCAAGCCTGCTG |
| | | AGGGGGCTCGGGCTGGTGGCCAGCAGGCTCGGCTGACCAGCAAGTGGGTGGTCCCGAGGCGGCCAGGCAGCCTGACCCAAGG |
| | | CAGCACGACCGTCCACGGGTCATCAGGCTCTAGACTCCGGGGTGGAGACGCGGGGCTGCGGGACCGAGTCGGTGCCACTTTC |
| | | TCGCACCCCTGATCCTCCGTGCCTCTTCCCAAGACCGCCAGACCCTTCACCGGCCCCTGCCACTGCCGGGAACGCTCCCGCC |
| | | TGCTCCTTAACTCACACAGGCAACAAGTTCTCGCTTCCTCAGAGCAGCCACGGTGTGCTCCGGAGGCCCGGCGCAAGGTGGA |
| | | AGCTCCCCGCGAGTCCGCGTTGCCCACGCCCCGAGGGGCGCCCGCTGCCGTCGCTGCCCCCCAGGAGCGTGGAGCTACAGGT |
| | | AGCGCCGGCAAGGGCTGCCCCCCGGGGCGCCCCTCCGACCGGGACGCTGCGGCATCCCCGGAACGGGCAACAGGTACGCGAG |
| | | CGCACCGAGGACCGCAGCCCCGGCTCCTACAGCCCCGGCCCTCGCCCCTTACCTCCAGCAACTGCACGTAGCTCGCCGGGAA |
| | | CCAGCCACGGAGCCCGTCCTCCTTCTCGCCTTCCCACCAGCCGCCGTCCGGGACCTGCAGCAGCGTGATCAGCTCGCCCGCG |
| | | GCGAAGCGCAGCCCCTGGCCGTGCCGCTCCCCGGAGAAGGGGTACAGGGTCCGGCAGCGAGCGCCGGACATGGCCTTGGCGC |
| | | CCGGGTTCACAGCGCAGCCTGCATTCCCGCCGAGCCCCACGGGCTGGGCAGCGGCTCCGCGGGGTCCCAGGCGCCCGGCGCT |
| | | CCGGGCTCCCGCGCTCTGGGCGCGCGCCGTCTCTGGGGTGCGCGGGGGTCCTCAGGCAGGCGGGGGACGCGCGCTCCGCGCC |
| | | GGGAAGCAGAGACTCGTTGGCTTCGCAGAGCGAGCGGCGACGCCCCCGGGCCGGGCAGCTCGCGGATCCTAGCTCTGGGCTG |
| | | GGGGGCGGGGCTCAGGGGGAGGAGACCCAGGCGCCGCGGGCTCCGGAGACAACTTCCCGGGAACGCGGAACGCGGGCTGCCG |
| | | CCGCGAGGGCTGGCCGGGGCCGGGGCCGGGACCGGGGCCGGGGCCGCCCTGGCGCGCGGAAATGCGCAGCTCCCCCGGGAGG |
| | | TGGGACCCGGAGCATCCCCCGCGGCAGGACTCCGCGGCGGGCGGCCGTGGAGAGGAGCTGGCGGCCGGGTGGCCGGGCGGGG |
| | | GTTAGCTCACTGGCGAGGGGGCGGGCCGGGGCGGGGCCAGGGCGGCCACGCCCGCGGAGGGCTGCGCGCTCCCTCGCAGAGG |
| | | GGCCTGTCACTGGGGAGAGGGGCTCCCACTCGCGCCCCCACCGCCGGGGACCGCGGGAATGGAAACGCCCCTCTGTTCTGTG |
| | | CCTGCTTTGTAGGCCTGGCGGCCCGTGGCGTGCGGGCGGCTTCTTCGAGGTGTCTGCGAGGGATCGTTTGTGGTTGTCATTG |
| | | TAGTGGTTGAACCTGGGGTTGAACTTGTCTCACCGGAAACTGAACAATGGGTGAAAGACAGGTTTGAAGCCTGGCGCCTGAG |
| | | CCCAGCAGCCTGGTGGCAGCGTGGCACGCTGGAGGGACGGAATGGAGAGAAGACGGGGTCGGAGTTGATAGAATTGGAAACA |
| | | GTCGATGGGGATAAATGGAG |
| 455. | GLUL | CAGCCTAGTGCCACTCTATCAACTGCTGCTGCTGCGGTGCCAAGTTTACGTGGGGGAAATGGAT |
| | | TCTGAAGTTTTCAGCAGTTTCTGCAGCTCCCACCAACTCCGGGGTCGTCCTCCCTGCCCCCTCACTCACCCCAGCCACAGAA |
| | | AGTCAAGCGTATCGTTAGGGTTGCCCTCTTTAACCCTTCTCTTTTTGGAGTGGCGTTCGCGCCTCTCCTCTTTCTCAGACTC |
| | | TAGCTGGTCCAAGCACCGGAGCTTTCTGGGAGAGGGCGATGGAGAAGGGGACAGACAGCGGCCGGCCCGGGGGGTGTCCGAA |
| | | CAGGCAGGTTGGTGGGTTAAGGTCTTAATCTTGACTCGAGATCTCTCCCCGGAGTTCACAGAGTAGGCGACGAAGCCGAAGC |
| | | AGCTGGAGCGCGACCCGGAGGAGTCTGACTTCTCGTTGTCTTCATAATTTTCATTCGTTGCTTTCTTCGTGGACTTGCGGCT |
| | | GGGGGAGGATCCCCGCTTCCCGCCCCCGAGGGAGTTATAATTATCCACTTTGAGAGCAGCCCCTTCCCACTGAAATGTCCCT |
| | | AAAGTGGGCGCCCTGCCCCTCGGGCCCCCGTCCGAATCTCAGGGCCTCACGGAGGTGGCCATACCCTGCCGCCGAGGGGTTG |
| | | GGGGGTCCGGGGGAGGGTGGGGGCGCAGGCCGTAGGGACTGCGCGCTGCTGCTGCCTCCGCCCGGACCGGCTTCTCCACTGA |
| | | CTGCGCCGAGGCCCGGGCTCTCCATTGTTCGGTCACATTGGAAGGCCCCCGGGACCCCGAGGCGGGAGCCGGCCGCCCAAAC |
| | | AACGCTCACCGTTACACGCCGCAATCGAGGCGTCTCAACCATCGCCAGAACCAGGACGATTGCTCCGAAGGCCCCACCACGA |
| | | GGAAGGCAGCCAGGCGCCCGCGCCAACCAAGCGCCTTGTTTGCGCTCCACAGGGGACCGCACCGCCACTCCATCCACAGCGC |
| | | AAACAAACCACTGCGAGCCCCAGGGCGCAGGCCGGCCCAACGGCCTCCAGGGGCGGCGCTTCGGCCGCAAGTAGTGAAAAGA |
| | | AGATCAAAACAACTCACCCCGCCCTTCCCAGAGCCTGGCCAAGGGCGCGCCCCACCAGCGCCAGCCCCGACGGGTCCAAGCC |
| | | ACCAGCGGCGGAAGCACGCGGGAGGAGCACTCATGCCGGATCTCAGGCATGGGCCGGAGAGGCCGCAGCGTAGGGCTGGGAC |
| | | TGGAGGGGCAGGCAGCCGAAAAAAGAGGCTGGGCGGCGCGCCTCGCGGGGTAGCAGGGGCGGCGCGCCTCGCTTGCGTGACA |
| | | CCGGGCCGTCCGGGGGCTTACCTGGTCGCCGAGCAGGCGGGCGGGTAAAGCTAGGCCGCGAGAGCGAGGTTAGGAGAGGAGA |
| | | GGAGGCCGCAGTACTGCTCACACGCTCCGCTCTTCTCCCACTCTCGACTCTGCAGAGCCGCCAGCAGCCCCGCACCCTTTAT |
| | | CCGCGGCCCGGGGCCGCCCCCGGTGCCCTGATTGGCTCCCAGCGGCCAGGAGCCCGGGCCGATCAGCTGATGCGGTCCCGGG |
| | | CCCCGCGGCCATTGGGCGAGGAGCCCGGCGGGCGCCGCTGGGGTGGGGGCAAGCGAACTGGGAAAGAAAGAAAGAAAGAGAA |
| | | AGGGGCGTGGGGGAGGGGCAGGGCGGCGTGGGGGAAGGGAGCGGAGCCGGGAGGGCTGAGGGCAGAGGGGCGAGTTGCCGCC |
| | | AGGCCGGGAAGCCTCGGCTCGCCGCCCACCCGCCAGCTCGCCAGGTGCCGCCCCACGGCCCCCAACCCGGGGTCCCGAGGCT |
| | | CCCAGCAAAGCCCGCCCTGGACCTCTGCCCTGCCGCCTTCACGGGGAATAGTTTTACGAGGAGAGCTCCCTGGGGAAATGCG |
| | | GAACCAGGGAAACCGAATTTAGCCTTTTGTTGCGAGGGAGCCTCCGAGCCCCGCTTCCTGCGGGCAGGGGTTAGCTTGAGAG |
| | | GGAGCGCGGGGCGCGGGACCCGAGGGAACTTTGGGCGGGCCTGGGCTAGGGCGGGTGCCGCGCGGGCCTGGGGACAGGCCGG |
| | | GGTTCCCAAGGCCTGGAAGAGCCGCCCGCTCCGGCTCTCTTGGGTCTGGGGAAATAAACAGAACTCTGCCATTTCACATTTG |
| | | AAATCAACGTGCTGTTTCTCTAATTTTACAGAGAGGATTCTGGAGGGCGGTAGAAACTAAGTCTGGAGACCTGTCTCATACT |
| | | GCTGGCCAGACCCTAGGCAGGTCTTTCAGGGCCCAAGTTTCCTCATCTGCAAAATAAAGTGGTCATCGA |
| 456. | GNG2 | GCAGCAGCAGTAGAAACAACAACAGCAAGAACAACAATAGCAACAAGCCAGGTTCAGGCTAAAA |
| | | AAGCAGGCAGTGTGCCAGGAAGGGGGCCGACTGAGCAGTGTGTGGAAGCTGTCGTGGAGGTGACCGTGGGGGGATGAGAAAA |
| | | GGGGAAGGAAGGAACAGACAGCACCAGAGGGACTTGAACAAAGGGAGAAGGGGGATTAAAGTCAAATCACTGTCAGCAGCCC |
| | | AGGAGCAGACAAAACCACCAGAGTAAGCCCTGAAATGGAAATGAGAGGGAAAAGAAGCAAGCCGCTAACAGCTACCAAGAAG |
| | | AGGAGGAAGTGGGAGATGCGCTGCTTCCTGTAGAAAGAACGTTGATTGAAGACAAAGCTGGGTGGGGACTAGTCCCTGGGCT |
| | | GCAGCCGCTGCTACACATACTCACAACGCTGCCGCCGCGCTCCGTGGGCAACTCCTACTACTGCTGGGCTGGGCTGGGCTGG |
| | | GCTGGGCTGCGCCGGAGCTCGCCTGCACAGATCAGCTCCGGAGAGGGGAAAACCACGCTCCTCGGACCAAGCCTCGGGAGCT |
| | | AAGCCAGATCTGCCAGTGAGCCTCAGGCTTTAGGAACTGAAGAGTGTTTCTGAAAGATCTATCCAGCACTCCGTGACTGGTG |
| | | CTTTCATATATTTGTGACAATTTTTGAGTAATATTGCATGAAAATGTCCCTATGTTACATCCATTCAGAAGTTTTGTTGTTT |
| | | TACTCTAAAGCTGGGAAAGGAAATGAGAGGGAAAAGACCCCGGAGAGGGAAGAAAATCTCAGTATTTTGAAAATTGAGATTA |
| | | CTTCAGAGCCTTAGCCACACCTAAAATACCTCCTAGTTAATAGTGGTATAAATGCCTCTTCAATACGTTTTCCAGAATCCAA |
| | | AGCATTTTGGTTTATCCAGGACCCAGGGCAGCACAGCTGTCACCAAGCAGGAGAGTTAAGGATTCACCATGAGCTGGGAAAT |
| | | GCTTTTGCCATGAGTATGAGCAAATTCCCTCTTTCCCTGAATCATGGACATTCTAGATTAAAAGAACATTTTTTTGTGCTCT |
| | | TAACAAGAAAACCATGGCCCTCCTTTGTTCAAGTATCAGAAGAAATAAACCCACAGCTCCAGAGAAGGTGACCATTCTCAG |
| 457. | GS3955 | ACACCAACTAAAAGGAAGCAATGGATTTCCTTTTAAATGGAGAGGAGGATGTCGGAGAGATACC |
| | | ATGAAACGAATTCTCGAAACTCCAGGCAGCAATACCGATGAAAGCGCATTCTTCCAAAACAGAGCTCCTCATTCAGGGCCAC |
| | | ATCTGAGCCTGGGGGCCCAATGCTCAGAGCGTGCCTGGCTTTCTGGGGAAAAGCGCCAGGAACATGAACGAGCTACGAAGAG |
| | | CTGGAGCGGGCGCTGCGGGAGCAGCGTGTGGGGAACGGCTCGGGAGTCGCTGCTGGTAACCCCGGCCCAGCCGCCCGCAGCT |
| | | GGGCTCGCAGCCGGCTCGCAGCCGACACCCAGGGTGCTGCGCCCGGGGCCCCGGGCCGCGGAAGGATATTTCACTTTGTTTA |
| | | CCTCCCCGGCCGGGCGAGGGAGCTGCGAGGCCGGTGCTGGCGCCCCCGGCACGGAGCTGCAATGGCAGCGGCGCGGGGAGCC |
| | | GCCCGAGGACTGGCAGCGGCGGGGCTGATTGATGGGCGCCCGGCGCAATGAGGGGGGCCCGGGCGGGGGCGGGGCGGGGCCG |
| | | GCTCTGGGCTGAGCCAGTCGCGGGGGCGGCGGGGGGGGGGCGGAGGCTCCGTGGAAATGTGTCGGTGACATTGAATGGGGAG |
| | | ACCGAGCGCGAGCGAGGCGCGCGCGCGCACACGCGCACTCACGGCCCCGCTCGCTCCGAGATCCCCGGCCACGTAAGTAACT |
| | | ATTAATACCGCCTCGCCGGGGGATGCGGGCGTGCTGAGCGCGGGGATTGGCCTCGGGCACCGTCGGCCGTCCCCTTTAATTT |
| | | TTAAATACACGGTCCCCTCTTTTCTCTGGGGGGGGCAAGCAAGAAATCAAAGAAGGAGGAGACAAGCCGTCAATTTTCTCCA |
| | | AAACAAACCCCACCGGGCAATTTGGTCTCGGGGTAGGGGGAGACGGGGTGATTGCAAATTATTCCAGGACGAGATCCAGTTC |
| | | TCCAGCGGGAAAGGGGCAAAGGAACGCCGCGCGTTGGAAGGGCCAGGGACGCAGCTCCCCTTGCAGCGCCCGCAGGACCCCC |
| | | GCAAGCTCGTGCCGGCGAAATCGGAGACCGCCGATCTGTCCTCGTTCTCTCCTGCACGTCTGGCTGCATTCGGAGGAAGACC |
| | | TGGGGCGCGAGCGAGCGGCGACAGCATGAGCCTGTGCTGACCTCCGCGCGGCGGGCCGAGCCCAGGGCTTTGTCGCGGTACC |
| | | TGCGCCCAGCCCGCGCCGCAACTCTGTGCCCAGCTTTTGCAATCTTTTGTTGGCAGCGCTGACCGCACCAAGTTAAATGCTC |
| | | CCTTGCAATTTTTCTTTTTTTTGTTTGTTTGTTTAATTTTTGGAGAGCTCGCGATCTTGGAAAAGCCTCAGACGCCATCTAC |
| | | AGTTAAAACGTAGGTAACTGCCCTCTCCCGCACCCCCCCCTTACACGCCCCCCACCCTTTCCACCAAAAAAAGGGGGTGCAG |
| | | CGCGGATTCTGGCTGCCGTGCGTCGCCAGCCGGTAGACCCGTGCTTGTTTCCTTTCTCTTTTTGTTTGGCTTCTAACGCGTT |
| | | GGGACTGAGTCGCCGCCGTGAGCTCCCCGAAGACTGCACAAACTACCGCGGGCTCCTCCGCCCCGTCTGCGATTCGGAAGCC |
| | | GGCCTGGGGGTCGCGTCGGGAGCCCTGGCGCTGCAGCTCCGCACCTTAGCAGCCCGGGTACTCATCCAGATCCACGCCGGGG |
| | | ACACACACACAGAGTAACTAAAAGTGCGGCGATTCTGCACATCGCCGACTGCTTTGGGGTAACAAAAAGACCCGAGTTGCCT |
| | | GCCGACCGAGGACCCCCGGGAGCCGGGCTCGGAGCAGACGAGGTATCCGGCGGCGCCCATTTGGGGGCTTCTAACTCTTTCT |
| | | CCACGCAGCCCCTCTTCTGTCCCCTCCCCTCTCGCTCCCTTTTAAAATCAGTGGCACCGAGGCGCCTGCAGCCGCACTCGCC |
| | | AGCGACTCATCTCTCCAGCGGGTTTTTTTTTGTTTGTCGTGTGCGATCCTCACACTCATGAACATACACAGGTCTACCCCCA |
| | | TCACAATAGCGAGATATGGGAGATCGCGGAACAAAACCCAGGATTTCGAAGAGTTGTCGTCTATAAGGTCCGCGGAGCCCAG |
| | | CCAGAGTTTCAGCCCGAACCTCGGCTCCCCGAGCCCGCCCGAGACTCCGAACTTGTCGCATTGCGTTTCTTGTATCGGGAAA |
| | | TACTTATTGTTGGAACCTCTGGAGGGAGACCACGTTTTTCGTGCCGTGCATCTGCACAGCGGAGAGGAGCTGGTGTGCAAGG |
| | | TAAAGGGCCAGTGGGTTGCTTTTTGTCTTTGGAAGGGGCCCGAGGGAGCGGGAGGGCGCCAGGCCCTCGAGTCTGGGAGAGG |
| | | GAGATTCGCGGGATAATTACCGTGGCCTTATTAAATGGGTTTATTTATTTATTTGCTCAGGTTCGGTAAGTTGCGAAGTTTT |
| | | TAGACCGTTTCAGACAATGGGGCGGGCGGCAGTGGGGGCGTTTCGGGGAGAGCCCGGGGAGGAGAGGGCGGCGGGACTGCGC |
| | | GGGGGCCACGGACACGCGTGCACCGAAGGCTCCAGGAGCTCTCTGCGCGAGGCCGGGTCCCGCTGCCCGGGGGGGATTTCTT |
| | | CCTGTGTCTAGCCCCCTCCCCTTCCAACAAGGATTAGGGAATCCCCCGGTAATTTTAAGACTGATGACTTCGTTCTTTTCGC |
| | | AGCCATTGTTCTTAGCAGCGGGCAGGTGTTAAACCTTTGTTCCGAAGGTGCCCTTTAAAACAGACACACAAAGGTGCCCCCT |
| | | TCGGCTGAGCCCAGGGGCCCAGC |
| 458. | GSTP1 | CGGCGCGCCAGTTCGCTGCGCACACTTCGCTGCGGTCCTCTTCCTGCTGTCTGTTTACTCCCTA |
| | | GGCCCCGCTGGGGACCTGGGAAAGAGGGAAAGGCTTCCCCGGCCAGCTGCGCGGCGACTCCGGGGACTCCAGGGCGCCCCTC |
| | | TGCGGCCGACGCCCGGGGTGCAGCGGCCGCCGGGGCTGGGGCCGGCGGGAGTCCGCGGGACCCTCCAGAAGAGCGGCCGGCG |
| | | CCGTGACTCAGCACTGGGGCGGAGCGGGGCGGGACCACCCTTATAAGGCTCGGAGGCCGCGAGGCCTTCGCTGGAGTTTCGC |
| | | CGCCGCAGTCTTCGCCACCAGTGAGTACGCGCGGCCCGCGTCCCCGGGGATGGGGCTCAGAGCTCCCAGCATGGGGCCAACC |
| | | CGCAGCAT |
| 459. | GUCY1A3 | CGGGCGTGATCTCACCATGGTGCGTTTTGGCTGCTACTTTCTTTTTCAGTCTTTCGGTGCGGAG |
| | | AAGGGGAGGAGGCGGGCAGAGGTCTGAAAAAATCGAATGCCTTATGGAAAGGAACTGCAAGGGTTCCTTTGGGGTGATCAAA |
| | | GAGGGAGACACAGACACAGAGAGACAAAGGCAAGGAGGACTGTCTGGGAGCCACGCGGGCGATACAGTTTCCGAGGCACGCC |
| | | GCGTCCCGCCTAGCCTGTTGAACAGGTAGACATGAGCGACCCAAGCGTAAGTGCCGCTGCCGCGAGTGCCCAGGGTGCACTC |
| | | GCGCGGGGACCGACAGGTGCCACTGCAGGGTCCCCTCTCCCGCTCACTGCCTGCCACCCCTGACGCCTGCTCTCTTTCTCCA |
| | | TTCGCAGTGCGGATTTGCGAGGCGCGCCCTGGAGCTGCTAGAGATCCGGAAGCACAGCCCCGAGGTGTGCGAAGCCACCAAG |
| | | TAAGTGGTCGCTGCATCCCCACGCGGGTCGGGGTTCTTCAGACAGGCGGTCCCTCTTCCGGGACCCCCGCTTGGGCTGGGTC |
| | | TCCTTCCTCTCCTGAGAAACCTTCGGTACGCTCCCAGTTGCCCAGCCCTGGGAAGCCAGCATCGGGTTCCCACGCTGGGGAG |
| | | GAGCGAGAGGAAAGGAGAGGACGCCCGAGGGGCCGGCCCAGGGCCCGGGGGACCCGCGGCGCCCGAGGGAGGAGCACAGACG |
| | | GGGGTGGGCGCCGAGCAGCGCCGCGCCGGCTGCTGAGCTCGCGGGGCCGGGCCGTGAGAACGCGCGGGGCGCGCCGGGGCCG |
| | | CTGGTCTCCCACGGCCAGGGCGCCTGGGCCCCAGAGCCCATCAGCCGCGTGCAGCGCTGCGGCCTCCGGCGCGCCGGCAGGC |
| | | AAGCGAGCGGGCGCAGCTGGGGGATGCCTGGACCCCGGAGGCAGCAGGGCTGGAGGCGGTGGCGGGCTTCAGGGCAACTTCA |
| | | CCCGGCCGGCTGCGGCGTGGGTAAGAGGCGCGTCCTCCTCCACAGCAAAAGCTGCGATCGCTGGGCTGCCCAGAGAAGGCAG |
| | | CCGGCTAACTGCCCAGCGGCTCCACGAGGTGTGCGCGTGTGTGCGAGAGAGACAAATGATTTACTGCTTAGCAGTCCCATTT |
| | | CTGTTTTCGTTAGGACTGCGGCTCTTGGAGAAAGCGTGAGCAGGGGGCCACCGCGGTCTCCGCGCCTGTCTGCACCCTGTCG |
| | | CCTGAGCTGCCTGACAGTGACAATGGTGTGAGGGGCTGCGTTGCAATGTGGTCATGTGATTTCAGGCTATGGATTTCGTGCG |
| | | TCTTTCTCTGCTCACTGATTTTCACATGGTTATTCTTTCATTTTGCACTCGATAAACAGTTTACATCTTCTGAAGTAAGCCA |
| | | GAAGATTGTATGGCTCAC |
| 460. | GYPC | CTGTATCCGGTAGTGGCAGATGGAAAGAGAAACGGTTAGAAAAGCGTGGTTCTTGCGCAGGAAA |
| | | GTTGGAGCAAAGAAGAGTTCAGAAGTGGGCGGGTGTGTGTTTAAAAAAAAAAAAGGGGGTGGAAACCCCACCAGCCAAGTCT |
| | | GCAGAAAAAAAATAAATGAAGTCTGCCTATCTCCGGGCCAGAGCCCCTCCCCTCGGCCCGCGCGGGAGGAGTGTGACCCAGG |
| | | TGCCGCTTCCTCTCGCCGCCGAGGGTCAGGAGCCCGGGAGCGCGACCCTCCCCCGGCCCGGCCTGGCCCGGCCTGGCCAGTC |
| | | CCCGCGGTCTCTGCCCGGGCTGACGCCCAGGAATGTGGTCGACGAGAAGCCCCAACAGCACGGCGTGGCCTCTCAGCCTCGG |
| | | TGAGTACCCGCCGTGGGGAAGGGTCCTGGGGACCCACTGGAGGCCGCGGCCCGCAGCAGCCAGGGGCCGAGCCACGGCCACG |
| | | GACGCCCTGGTGTCCCGGTCCGTGCCGGGCCTCCAGGCGGAGGAGGCGTCCGCTGGGCTCAGATCCCCGACTCCAGCCCCGG |
| | | TTCCCCGGCGCCTGGGCTGCGCGGAGTCCCTGTCCCGCGTCCCGGACCCCTACGCGCAGCCTCCACGCGCTCCGAGCTGGAG |
| | | AAGCCGCCAGCCCGCCCTCCCAGGGCGTGTCGCCCGCTTTCTGTTTCTTTGTGCGGGGCATGTGAAATGTGTGGGGCCAGAA |
| | | TTGTCTCCCTAAAGAACAGTTAGGTGAGAGTTCACATCACAAGTTGCTTCTGTGCTGCTCCCCAGCCAGGGTCCGACCGGCC |
| | | AGGCTCCGGTGAACTCCAGGCAACTCCAGCCTTGCCCAGGGACTTGCTCGCTCCGCTGGGCGCCGCAAAGGC |
| 461. | HIP1 | ATCAGGCTCCTCTTGGCCACGCATCCTGCCATCTCCTCCGAGCCCGCCTCTGCCCTTCTCCCCAG |
| | | GCCAGCTGGGGTACCCTGGGGTCCCCGACCTTCCTGGGATCGCCCAGATCTGCACGTCAATGCCCCCGCTCACTACACCCTC |
| | | GCCCCGTCTTCAACTGGGGCCTGCAAGACTCCTACTCCCTTCAAAGCCCCTCCCGGACACCGCGTCCCTCAGGGTGCCCCAG |
| | | GGATGCCCCGGGGTCCCCCGTCCTCACCTGAGTCCGCTCGAAGCTCTCGCGCTCCGCCGCCTCCAGCCCAGCGCCGACCCCG |
| | | CGCCGGCTCAGCACCTTGGGCAGTGGGTTGGGCACCTGCTTCATGGAGCTGGCCATCCGATCCATGTCGCCGCGAGGAGCCG |
| | | AGTCAGGGGCCCTCGGCTGCCCCGGGATCCCCACGGCGCCGCCCGCCCCGCCCTACCCGCGGGGATCCGGCGCTAACGGGAC |
| | | GGCTCCCGCCTGCCATTGGCTGTGAGTTCTGCCAGTCTTCCAGAGACCCCGCCCCTCGCCGAACGGCTTCCCCGCCCGCCCC |
| | | CGCCCCTTCAGGAGCCCCTCCGCTGGCTCGGCCCCGACTAGCCTCTCGGGGCCTCGAGGCGCGTGGTGACTGGTCTGGAGTC |
| | | CTGTCAGTCACCGTGGTCCCGCCGTTCTAGGCAGGGCCGGGGCGGGGTGCGGACGGGGCGAGGCCTGGATGGGGCGTGGCCT |
| | | GGGCGGGCTGCCCTGATATGCCCTCGCTCGCCCCGCCCCCGCCCGCGCACCGCCCTCTCCTCCCCTCGGCCGCAGTCCCCGC |
| | | GCGCCCCGAGCGTGCTGCCCTCCGCCAAGCGCCGCCCACTACCCTGCCCGCTCCTGCAGGGGGCTATCCCGCGACGGCCCGT |
| | | GGAGATGGGCGGGGATGACGCGGGCCGGGCAGTGGGGCCTCCCCCGGGGGAATCCCAGCCCGCCTGCGAATAGCCCGTTAGC |
| | | TCCTTCCGGGCCTGGGACCCGGGAGCGCCGGACTGACCAGCCCTGCGGCCGCAGCCCGGGAAAGCGCAGCCCCGCGGGCGGG |
| | | GCGCAGCGTGGGCGCCTGGCCGGATCCGGCTCAATGGGTGGCCGCCCCCACCCAGCTCCCAGGCTTCCCCGTCCTGCACTTC |
| | | TCCATTCTGCCCCTGCCCATGCCCTCTGCAAGGATCGCAGAGCCCAGGTCCCGGAGTTCGGGGCTGAGTCCGCGGGCACCTC |
| | | CCGGGACCTGCCTCCCAAACCTCATCAAAGGAACCCGTCAGAGCTTATTTGTTGGTAGAAAGCCCTGTAGACACTTTCGGTT |
| | | TATCTTAGGCTCTGAGCCGCGCGTCCCATCCTGCTCAGACGTCAGCCAGGGTACCAGCAAGCAGAGAGAAGTGTGATGCTTA |
| | | GCAGGAGTGCTGGAGAAAGTTAAAAGTTTTCCGTTAAAC |
| 462. | HOXA1 | TTTTCCTCTTCTCCCCCTTTCTCCCTCCCTCTCTCTTCTGTCCGCCTCCCCTTCTCCCCTCAGC |
| | | CTCTCGCCCCCCTCCCAGTGTCCAGCCCAGAGTCTGCGCCCCGGGCCCATTGTTAGCAGGCTATTCCACGGCAGCTTTGCAT |
| | | CTGGCTCCGGCGGGAAGCGGAAAACGGGAGGCGGCTCTCGAAGCTTCCCGACCTTCCTGCGCCATCAACTTCTCAGGAGTGG |
| | | CTGGAGAAAGATGCATGTGCCAAGCGAGACAACAACCCCAGGTCCATGTGTCCAAATCCCCGTAGCCAGGGCGGCGGCCAGC |
| | | CAAAGAAATGCCGCCCCGAGCAGGCGCGTGCGGCTCCTGGCATTCTGGGTTTCATACCCGTAGGGCTCGGGTGCGGTGAGTA |
| | | TTTCCGATTTCCAGGAAGTCTGTTGGAAGTTACCAGCAGGGAAAGAGAAGATGCTTGCTCCTCTCTTTCCCTCCCTCTCTCT |
| | | TTTTTTTCTACCCTTCTTCTTGTC |
| 463. | HOXA10 | TCCCTCAAGTTTCTCCAGGAGCCAGGGATAGATAGCTGGGCGATTCCGAGGTCCAGGGGAAGGG |
| | | AAATGGCCCTTCTCTGGCTCTCAGCTCAGGGCCCCCCGCTTCCAGGCCGGATTTGCCTTTTCTTCTTCCCGCAACGAAGATT |
| | | CCCGCCCCTCAGCAACTTTGAAAAAAGCATGGGGGATCGTAAACTCGAACTTCGCCGGTTAATGGGCTTATTTATTGGCGCT |
| | | GGCGGCTGCTTATTTTGGATGCCTTACAAACATCCGCGCTATCTGCGGGCGAGCTACTTTCCCTCCCTCCCCCTCCCCCCGC |
| | | GTGGGCCGCGCCGCGCAGGCTGGGCAGGGACCAGGGCTCTGGGTCCTCCCGGCCACAGGAAAGAGCGCACAGGAGGGGGCCT |
| | | GCTCGCTGGTGTCCTCGTCCCTAGTCAGGGGGAGCTGAGGCCAGCGCCGAGGACGTCTTGCTGTGGGGCGCTAAGCCGGACA |
| | | TGAATTTTACTGCGTCCCCACGCCCAAATATTAAAAAGCAAGTTCACAAGGTCAGCCTGCCTGCAGCTTGGGCCAAGGCCGG |
| | | CCGGCTGCTGCGCGGGCTCCTAGTTTTCTGATCCTTCTCCTCCTTGTGTCTGCCTGTCTGCCCGCCTGACTGCAGCCCTCTG |
| | | CAGCCCTGCTTACCCAGGGAATCCTTCTCCGGCGAGGCTTTGCTGCTCTCGGAAGGGGCCGGGGAGAGCTCCTCCGCGGCCG |
| | | AGGACGACGCGTGCGCCTCCTCGTCGCCCTGCGAGCCCCCGCCGCTGCCGCAAGCCAGCGTGGGGGGCGGCGGCGAATCGAG |
| | | GGCTCGCTCCTTCCGGGCCGCATCGGCCGAGCCGGAGGCTAGCGCGGGCGGGAGATCGAAACCGCGCCCCGGGGGCTGCGCG |
| | | GGGAACGGGCCAGCCCCGAGTTGCTGCGCGCCGCCGCCGCCGCTGCCATAGCCCTTGGCGGTGCCGTAGGCCTGAGAAAGGC |
| | | GGAAGTAGCCAGGCACTGGCACCCCGCTGGAGGTGCCCAGGGCGCAGCCGTCGGGCGGCGGGCCCCGCGGGAAGGGAGCCAG |
| | | TTCGGCGGCGGTGGCCGAGACTTTGGGGCATTTGTCCGCCGAGTCGTAGAGGCAGTAGGAGCTCTCTTCTTTGATGTTCTGC |
| | | GCGAAAGAGCACGAGGTGGCCTGCGGCGCTGGCTGGGGTGGTTGCGGCGGGGGCGGCGGCTGCTGCTGGGGCGGCGGCGGCG |
| | | GCCCGTCAGGCGGCTCCATCCGGCAAGACCGGGGCGCGTCTAGCCACAGGTCTATGGGCGAGGGCCCGTAGCCGTGCGCCCC |
| | | GGGACCTAGACCCCCGCCACCGCCACCGCTGCCCGGCGACGCTGCCTCATTGCGCTTGCCGCCCAGCGTGGGGAAGAGCCCG |
| | | CAGCTCTGCAGCCCGTAGGGCAGGTCGGCGGCGGGCGGCAGGTAGACCCCGCCGTGGGCGTAGTAACCGCCACCGCCGCCGC |
| | | CCCCCGCGCCACCACCACCGCCGCCTGCCTCGCCTCTGCCCGAGCTGATGAGCGAGTCGACCAAAAAAGAGTTCGCGGCGGG |
| | | GCTCTCCGAGCATGACATTGTTGTGGGATAATTTGGCGAAGGGAGCAGATAGCCCTTTCTGGCTGACATTTCTTGTGCAAAA |
| | | CATGCTGAATACGATTAGCAATCCCCCCGCACCGCGGCGGGCGCCCGCAGCCAATCCCGAGCCAGAGTTTCCGCGCGACCAC |
| | | TCCCAGTTTGGTTTCGTAGGCGCGGGGCCGCTCTCCGAGGGCGCCCTCAGAGCCCGCGATTGATATAAATATGTAATCTGTA |
| | | TTGATGGGCCAGGAGACGCACCCCGACACCTTGGCCCGAAGGCCGGGAGCTGTGGGGGCTGCCCCAACGTGGCTGGTGGGGG |
| | | GCCTGGCCATTGGGCTCGCCCCGCCCCTACCCGGACGTGAGCCCCATACCGGGGTCCCTTAGAAGGGCCCTTGGGCCCCGCG |
| | | CAGTTAACAAGTGGGGTGTTTATGGTGCGCGCCCAGTCTGCCTTGGGTGCTCACCATCCCTGTCGCAGAAGCTGCCACTAGT |
| | | CCCCGGTGTACTCTAACCACTGAAGCGGCCGTGTCGGGGACTCACGCGCTTCCCATTCAGCTCTGGATCTGGAACTGGCCCC |
| | | TTGTCTGAATTCTGCCTCCTCAAAAGTGGCGAACCTGGCCCTATGGCCGTCAGGATCCTCAGAGTG |
| 464. | HOXA10 | CCAGGGCCCTGATTGCCCAAGACTCGATAAGGGGAGAAAGAAGGGCATCATTGGTCCAATGGGG |
| | | AAGGCAGGAAAAACCGATTCGGGGGTCAAGGGCCCTCCCTCAAGTTTCTCCAGGAGCCAGGGATAGATAGCTGGGCGATTCC |
| | | GAGGTCCAGGGGAAGGGAAATGGCCCTTCTCTGGCTCTCAGCTCAGGGCCCCCCGCTTCCAGGCCGGATTTGCCTTTTCTTC |
| | | TTCCCGCAACGAAGATTCCCGCCCCTCAGCAACTTTGAAAAAAGCATGGGGGATCGTAAACTCGAACTTCGCCGGTTAATGG |
| | | GCTTATTTATTGGCGCTGGCGGCTGCTTATTTTGGATGCCTTACAAACATCCGCGCTATCTGCGGGCGAGCTACTTTCCCTC |
| | | CCTCCCCCTCCCCCCGCGTGGGCCGCGCCGCGCAGGCTGGGCAGGGACCAGGGCTCTGGGTCCTCCCGGCCACAGGAAAGAG |
| | | CGCACAGGAGGGGGCCTGCTCGCTGGTGTCCTCGTCCCTAGTCAGGGGGAGCTGAGGCCAGCGCCGAGGACGTCTTGCTGTG |
| | | GGGCGCTAAGCCGGACATGAATTTTACTGCGTCCCCACGCCCAAATATTAAAAAGCAAGTTCACAAGGTCAGCCTGCCTGCA |
| | | GCTTGGGCCAAGGCCGGCCGGCTGCTGCGCGGGCTCCTAGTTTTCTGATCCTTCTCCTCCTTGTGTCTGCCTGTCTGCCCGC |
| | | CTGACTGCAGCCCTCTGCAGCCCTGCTTACCCAGGGAATCCTTCTCCGGCGAGGCTTTGCTGCTCTCGGAAGGGGCCGGGGA |
| | | GAGCTCCTCCGCGGCCGAGGACGACGCGTGCGCCTCCTCGTCGCCCTGCGAGCCCCCGCCGCTGCCGCAAGCCAGCGTGGGG |
| | | GGCGGCGGCGAATCGAGGGCTCGCTCCTTCCGGGCCGCATCGGCCGAGCCGGAGGCTAGCGCGGGCGGGAGATCGAAACCGC |
| | | GCCCCGGGGGCTGCGCGGGGAACGGGCCAGCCCCGAGTTGCTGCGCGCCGCCGCCGCCGCTGCCATAGCCCTTGGCGGTGCC |
| | | GTAGGCCTGAGAAAGGCGGAAGTAGCCAGGCACTGGCACCCCGCTGGAGGTGCCCAGGGCGCAGCCGTCGGGCGGCGGGCCC |
| | | CGCGGGAAGGGAGCCAGTTCGGCGGCGGTGGCCGAGACTTTGGGGCATTTGTCCGCCGAGTCGTAGAGGCAGTAGGAGCTCT |
| | | CTTCTTTGATGTTCTGCGCGAAAGAGCACGAGGTGGCCTGCGGCGCTGGCTGGGGTGGTTGCGGCGGGGGCGGCGGCTGCTG |
| | | CTGGGGCGGCGGCGGCGGCCCGTCAGGCGGCTCCATCCGGCAAGACCGGGGCGCGTCTAGCCACAGGTCTATGGGCGAGGGC |
| | | CCGTAGCCGTGCGCCCCGGGACCTAGACCCCCGCCACCGCCACCGCTGCCCGGCGACGCTGCCTCATTGCGCTTGCCGCCCA |
| | | GCGTGGGGAAGAGCCCGCAGCTCTGCAGCCCGTAGGGCAGGTCGGCGGCGGGCGGCAGGTAGACCCCGCCGTGGGCGTAGTA |
| | | ACCGCCACCGCCGCCGCCCCCCGCGCCACCACCACCGCCGCCTGCCTCGCCTCTGCCCGAGCTGATGAGCGAGTCGACCAAA |
| | | AAAGAGTTCGCGGCGGGGCTCTCCGAGCATGACATTGTTGTGGGATAATTTGGCGAAGGGAGCAGATAGCCCTTTCTGGCTG |
| | | ACATTTCTTGTGCAAAACATGCTGAATACGATTAGCAATCCCCCCGCACCGCGGCGGGCGCCCGCAGCCAATCCCGAGCCAG |
| | | AGTTTCCGCGCGACCACTCCCAGTTTGGTTTCGTAGGCGCGGGGCCGCTCTCCGAGGGCGCCCTCAGAGCCCGCGATTGATA |
| | | TAAATATGTAATCTGTATTGATGGGCCAGGAGACGCACCCCGACACCTTGGCCCGAAGGCCGGGAGCTGTGGGGGCTGCCCC |
| | | AACGTGGCTGGTGGGGGGCCTGGCCATTGGGCTCGCCCCGCCCCTACCCGGACGTGAGCCCCATACCGGGGTCCCTTAGAAG |
| | | GGCCCTTGGGCCCCGCGCAGTTAACAAGTGGGGTGTTTATGGTGCGCGCCCAGTCTGCCTTGGGTGCTCACCATCCCTGTCG |
| | | CAGAAGCTGCCACTAGTCCCCGGTGTACTCTAACCACTGAAGCGGCCGTGTCGGGGACTCACGCGCTTCCCATTCAGCTCTG |
| | | GATCTGGAACTGGCCCCTTGTCTGAATTCTGCCTCCTCAAAAGTGGCGAACCTGGCCCTATGGCCGTCAGGATCCTCAGAGT |
| | | GTCAGGAGCCCAGAGTGAACTAGAAGCTGACTTGCCTCTACTTCCAGTATCCACAGATTTTTCCCCAAAATGCAGTGGTTGT |
| | | TCCCTAGCCCCTAACCCCC |
| 465. | HOXA11 | GTCCGGGCCTTTTCCCCCCCCCTTTCCTTTTTTTTTTTTCCTCCTCTCCCCTCCCTCCCGGCTT |
| | | CCTTTCTTTGTAGCCACCTCAGGGGAAGCAACAGATCGTCACTCGGTGTTCTCACCGAAAGCACGTAATCGCCGGTGTAACT |
| | | CATGTTGGCTGGGGGGCCTCCCGGCGCGCGCGGAGAGGCTGGGGTGCGCCCCCATGCAGCATGCTTGTGCTCAATTGCAGGG |
| | | TCCTCGTTCTCGAGTGTGCAGAGGGCGGTGAGAGCTCAACTCTCGTCCCCACCTCCCACCCGCAGCTCCCCGGGTGGGTGAG |
| | | GGATGCCCTGGACTGGGGATAGCCAGGTGGGAGTCCGTCGCTGTGTGGCCTGTGGTCTCGGAGTCTGTTCTCCTGGAGTCTC |
| | | GCATTTGCACCCCCTTCTTCGCAGTCCCCCTCCCATAGACTTGCTCTGGGAAGCGCCTCTGCCTCCGACCCTAGCCGGAACC |
| | | CCTTCGGGGCCAGAGTTTGAAGCCGTGGATGTGCCTGCCTGGTGGCTTGTCCGATTTGCACGGTGACTTGATTACACTCTCT |
| | | CATTCATGGTCACTTCCGAAGCGCTTTAGTGCCTTCCGTCCCTAAACCGCCAACAGCCAGAACGGCTTCTCCCCGCGGTTTG |
| | | TCACTGATCCGCAGGGCCCGGAAGGGCCTTCGTCTTACCCGGGATCCACCTCTCCCCTCATCTTCCCTGCCTACCTCTTCAT |
| | | CCCACCTTCTGTCCTTGGAGAAACTCCCTCCTCCTCGCTGCCTGCCGGGCTTCGGAGTG |
| 466. | HOXA3 | TGAGGTGCGGGCTGAGGCGGCTGTGGGGCAGGGGGCGCGGCCTGGGGCGGCGGCGGGTGCAGGG |
| | | GCGGCTCTCCCAGGCTTGGAGGCTGGCTAGGTGGGGCGCTCAGGGTGCGCAGGCACGCCTCACTCAGTTCGTGTGCCTTGGG |
| | | GTGGCCCCCGGCGCTGGAGGGAGACTGGAGGGAGCAGGCGGGTCGGTGGTACTCGCCGTCGGCGCCCAAAGCGGCGGACGCC |
| | | GGGTACGGCTGCTGATTGGCATTATAAGCGAACCCGTTGGCTGCCTGGTAGGGGTAGCCACCGTAGATCGCCGAGCTGTCGT |
| | | AGTAGGTCGCTTTTTGCATCGCGTTGTTTCACGATCTTGATCGCACACTCTGACAGGGGTTTGACACCCGTGAGGGCGCACA |
| | | TTGGCACGCCCCCGCGGTCACGTGACACTCCGCCGCCAATGGCCGCCCCGCGCAGACCTGGTGGGGCGAGAAGCGCAGCGCG |
| | | GTGAGGGCTCCGCGCAAATCCATCTTACTCTCAATAGCTAAGTGACATGAAAGCCATAAAAGAAAAAGTGGTCAGCAATATT |
| | | TAGCAGCACGACTTGGCCCCGGGCGCAGGGAG |
| 467. | HOXA4 | ATTTATGGGGGCTATAATTACTGCCCTAACAGTTTGGCGTCTCGTAAATCTCCTGATAAAGGGA |
| | | CCCTGGGTACAAAAGGGTTCTCATGTTGGGATCAGGCGGCTGGCTGGCGCGCACATACCCACATCTCACCGCAGCCCGGGTC |
| | | AGATGGGGGCTCCCCTCCCGAGGCCCCCTTCCCCTGAGCCTCTCCCTCCTGACCCCGACCCTCGAACCCAGGCCCAGCCCCG |
| | | GCCCACCTCCCGCGCCTCCCAAGCGGCGCCACGTACCGGCGCTGACATGGATCTTCTTCATCCAGGGGTACACCACGGGCTC |
| | | CTTGCCCTTCAGGCCCAGCGGGCTCTTGTCGGCCAAGAGCAGCGGGCACGCGGGGGCGCTGCCCCCTGCCGGGACGCCTGGG |
| | | GTGGCGGGGGCCGCCTCGCAGCGCCGCGGGGCCGCTGGGGGCACGGCGCGAGGCTGCAGGGGCGGCGGCAGCTGGGGCTGCA |
| | | GGACGTGGCTCGCATGCAGGCCGTGCGCTGGGCCCTTGGCTTGCGCCGGGGGCTGCTCGGGCTGGGGCGGCCGCCCGGGGCT |
| | | GGCGCCGCCGCGGTAGCCATAGGGGTAGGCGGTGTCCGCGGCCCCATGCGCGGGGTACAGCGCGGCAGCAGGGTAGGCGGGC |
| | | TCGCGGGCGGTCCGCGGCGCGTAGTAGGAGGCAGTGGGCTCTCGGCCGCCGCCCGCGTGAGGGAGCTGGGGCTGCTGCAGCG |
| | | GCAGGTGCTGGGTCGGGGGCGCTGGGGGCTGCTGGTAGCCGGGGCCCCCGCCCGGGCCGCCGTCTGCGCCGCCCGAGCCGCT |
| | | GTGCTGCGCGTACTCCTCGAAGGGAGGGAACTTGGGCTCGATGTAGTTGGAGTTTATCAAAAACGAGCTCATGGTCATTAAT |
| | | TTGTGAAGTGCAAAAATACTAATTTTTCTCGCGTTGTCGTTTTTTCTGGGCTTGCCGAGGCCCCTCCCCCTCCTGCCTCGCT |
| | | TCCCATCCCCCTTTCCTCTGCGCCCTTCCCCTCCCCCCGCTGTCAAGTGCCCACTCCTCCCCCTCCCGCAGACGCCGCCACC |
| | | AAAGTTCGAGCCGCTCCTCCCCAGCCCAGCGCGCGCCCCGCCCCGTGCCCCACGTGCAGCGCCCCCACCAATGGGCGCACCG |
| | | CGCGCGCGGACCCGGATCAGGAAACGCGCGGGTGCGTGATGGATGCTGCTGTCCGGCCCCTGGGCTGGGGGAGGGAGCAGGA |
| | | GCTTTGGACCCCAGCCCCCCAGCTTTGGTTCCCGCTGGGAATTCAGGCCCTGTC |
| 468. | HOXA7 | CAAGACAAATGGAGTTAAATAAATTTACGAGGATCGAACCCATTAATTGGGCCATAAAAAGTTT |
| | | TATGAGCCTCATTTACATACAATGCTATGGGCTCCACGCAATGGCGCCTCCGCTCCAATTAAAACCAGAAAGGCTGCGCCGG |
| | | GAGTCACGGGGCTACCGGCTCGCAACAGCCTGGCTCCGCTCTTCCGGCCCCGCGCCCCGCGCTCCGCGCTCCCCAGCGCTGC |
| | | GCTCCCCGCTCCCGGTCCCGCTCCGCCAGCCTGGCCCGCCTAGCGACTGCGCCTACCTGAAGACCGCATCCAGGGGTAGATG |
| | | CGGAAATTGGCCTCAGCCGCGCCATGCAGCGCGCCCTCGTCCGTCTTGTCGCAGGCGCCTTTGGCGAGGTCACTGCAGAGCC |
| | | CGGGGATGTTTTGGTCGTAGGAGGCGCAGGGCAGGTTGCCGTAGGCGTCGGCGCCCAGGCCGTAGCCGGACGCAAAGGGGCT |
| | | CTGATAAAGGGGGCTGTTGACATTGTATAAGCCCGGAACGGTCGAGGCGAAGGCGCCGGCGCCCGCCCCGTAGCCGCTTCTC |
| | | TGTGAGTTGGGAGCAAAGGAGCAAGAAGTCGGCTCGGCATTTTGGAACAGAGAAGCCCCCGCCGTATATTTGCTAAAAAGCG |
| | | CGTTCACATAATACGAAGAACTCATAATTTTGACCTGTGATTTGTTGTCCGGCAGCTTTCAGTGTCGGTTTTACGAGGTAGA |
| | | GTGATATATGATAACATTACACCCCCAGATTTACACCAAACCCCATTTTCTTTTGGACGGAGCTCGCCGCAGCACGTGACCG |
| | | CCCACATGACCGCCTCCGCCAATCTCAGCAGTCCTCACAGGTGGTCTCGCTCCGCAGGGCCCGCAGCCGCCTAGAATGGAAG |
| | | GGCAAGAGGCTCAAATATGCGGCCAAAGAATCCGCCCGCGCCCGGCGGGCCTGGCGCGTCCCGCGGAAAAAGACCTGGAGGC |
| | | TCCGCGGGAGCGCCCAGCTGGCGGCCAACCTCCGCACTGGGGTCTGCGGACGCCAGGCGGCCCGGCCCCACGCAGCACCCCC |
| | | CACCCCGCCCCCCCGCCGACTCCTGCTAGTGAGCCCTGGACCAAGCTTGGGATCCTCCCCATCCCTCTCCTGTCCGCCTGCC |
| | | CAGACCCTGGAAGGGTCTCTGTCCCCCGCAACAGCC |
| 469. | HOXA9 | AAGATTAGGATTCCTCTGTCCCGTTCACTGACTTCGTCTTTCTTTCCCAACCTGTCCCTCTACG |
| | | CCCCCCACTCCTTATTTAACCTTCCTGGAAGGCCTTCGGAGCTGGGCAAGCCGTCAGGGCGCCCTAAGGCCGCTGATCACGT |
| | | CTGTGGCTTATTTGAATAATCTGTCATGGGGACCCTTGTGGCCCGGGTCGCCCGCAGCCTCATCTTGGCAGGATTTACGCCG |
| | | CCACTGGCCGAAGGCAAGAAGTGGAAGGAATCGGCCGTCTCCCCCAGCGTCCCAGCTCCGGCTGCCCTGGCTGCCGCCGCTC |
| | | ACGGACAATCTAGTTGTACAAAAGGCTCTCTGGGCTGCACTGCTTTCGAAGAACGGCCCAAAGTATCTCGGTCCTGGGCCTG |
| | | GGCAGCCAAGGAGAGGGGCGGCCAGTCTTGGCTCGTCCCGAAGTGCCCGCCCCGCCCCCTCTCGCTGCAGCAGCCGCCTCCT |
| | | CTCCCGTAGCCCTGCGGGCCGCTCTTCACTGCTCTCCAGACTTGGGGCCCTATCTGAGGCGTCCCAAACACCAACTTCTGGC |
| | | TCCTGGCCCCAACTCGAGAGGCTTCCAGCGAGGACGAAGGCAGGCTCGAGAGAAACCTGGCGGGCCAGCAGATCCGGGAGGC |
| | | CGGCGTGGAGGCGGCGGCGGATTTGAAGGGAGGAGACACTTACTGGGATCGATGGGGGGCTTGTCTCCGCCGCTCTCATTCT |
| | | CAGCATTGTTTTCAGAGAAGGCGCCTTCGCTGGGTTGTTTTTCTCTATCAACTGGAGGAGAACCACAAGCATAGTCAGTCAG |
| | | GGACAAAGTGTGAGTGTCAAGCGTGGGACAGTCACCCCTTCTGGCCGACAGCGGTTCAGGTTTAATGCCATAAGGCCGGCTG |
| | | GAGGGCAAGCCCGCGAAGGAGAGCGCACCGGGCGTGGGCTCCAGCCAGGAGCGCATGTACCTGCCGTCCGGCGCCGCCGCCG |
| | | CCACGGGCGCCTGGGGGTGCACGTAGGGGTGGTGGTGATGGTGGTGGTACACCGCAGCGGGTACAGCGTTGGCGCCCGCCGC |
| | | GTGCACTGGGTTCCACGAGGCGCCAAACACCGTCGCCTTGGACTGGAAGCTGCACGGGCTGAAGTCGGGGTGCTCGGCCAGC |
| | | GTCGCCGCCTGCCGGGGAGGCTGGCCCAGGGTCCCCGGCGCATAGCGGCCAACGCTCAGCTCATCCGCGGCGTCGGCGCCCA |
| | | GCAGGAACGAGTCCACGTAGTAGTTGCCCAGGGCCCCAGTGGTGGCCATCACCGTGCCCAGCGCCTGGCCCGCCCGGCCCGA |
| | | CCCACGGAAATTATGAAACTGCAGATTTCATGTAACAACTTGGTGGCACCGGGGGGGAAGTACAGTCACCTAATAAGTTGCC |
| | | GGCGCCCGCGCCCCCATTGGCCGTGCGCGTCACGTGCCCGTCCAGCAGAACAATAACGCGTAAATCACTCCGCACGCTATTA |
| | | ATGGTCCGATGTTTTGCAGTCATAATTTTTATAGCAAAAGCCATATGTTTTTATGTAAAGGGATCGTGCCGCTCTACGATGG |
| | | GGTTTGTTTTAATTGTGGCCAACGACGATTAAAAGATCAAATCTAGCCTTGTCTCTGTACTCTCCCGTCTCCCCCCCCATAC |
| | | ACACACTTCTTAAGCGGACTATTTTATATCACAATTAATCACGCCATCAAGAAGGCGCGGGTCCCGCGTGCGAGTGCGGCCA |
| | | GCGGAGCCCCTCACATAAAATTAGACAATAATTGAAGCCATAAAAAAGCAGCCAAATCGCATTGTCGCTCTACTGTATTTAA |
| | | ATCTATATTTATGATATTTCATAAGGAGTTATTGTTTCAGAAGCCACACAGGCTGGCGGGAAGTCGGAAACGACCAACAGAT |
| | | TCGTTTGCCTCGCCGTGGCTCCCAGCTGTAAAAATTTACGAGGACTTGGAAAGGTTAGACTGTTGTGTTTGGTTGGCGAGCT |
| | | CCCTGTAAATAATCCCTGCGGTCCCCGGGAGAGGCGAGTTTACCCGCGGCCGCCCTCGAAAAGTCAAATTCAACGCAGGATC |
| | | CGTCCCAAACGGAGCCGCCGCCGGCCCTACCAGGGCACTCCAGGCAGGGACCGGCCGCTCAGGGAGTACCGCGGGTGTAGGT |
| | | CCCCACAGCTACCCGCCTGGAGCGAGGGGCGCCCGGGCAACCCTTAAATTCGCCTTTGCTACGAGGACCCCACGGAGGAGCT |
| | | GGCCAGGAGGGAGCGGCCAGCCGCCACCAGGGCGAAGGTTTTGAGGGCCTGGTTGGTTGTGCGGCGCGCTCGGTCCCCGGCC |
| | | CTCGACCCCACGCACACGCGCGCCCAGCCCGCCTTTCTCATCAGCTGGCAATCAGGATTCCCAGGCGCAGGCGGCTGGCGAC |
| | | CCAGCCCTGTGCTCCAGCCTCAGAGGCTCTAACCATGAGCGCTGCAAGCCTGGTTGCGCTCCGTGAATCCCAGCTGGGGAAA |
| | | AAACTACAAGTGGCATGAATGGAAGGCAAGTTCGGTTTGGGAAAAGGCAGCCTCGCCTAAGAGACCCCGCAGCTCCGGAACC |
| | | TGGGAGGCCCGCACCGATGTGGCCTGTCCCGGGGCCGCGTGAGCCTTTCAGGGCTCCTTCCTCCCTTTCCAGCTGCTACTCC |
| | | GGGCCTCGCCTTGGTTACCTACGGGGCCCGGAGACTCGGCGGAGAGGTACAAGGCCCAAAGAGAGGCAGCCACAGCTCAAGG |
| | | CCAGGGCTGGAAATTAGAACGGGGAGGGGTAAAAGGGCATCGACTCCAGTCCCATTCCT |
| 470. | HOXA9 | GGGAGGCTGGCCCAGGGTCCCCGGCGCATAGCGGCCAACGCTCAGCTCATCCGCGGCGTCGGCG |
| | | CCCAGCAGGAACGAGTCCACGTAGTAGTTGCCCAGGGCCCCAGTGGTGGCCATCACCGTGCCCAGCGCCTGGCCCGCCCGGC |
| | | CCGACCCACGGAAATTATGAAACTGCAGATTTCATGTAACAACTTGGTGGCACCGGGGGGGAAGTACAGTCACCTAATAAGT |
| | | TGCCGGCGCCCGCGCCCCCATTGGCCGTGCGCGTCACGTGCCCGTCCAGCAGAACAATAACGCGTAAATCACTCCGCACGCT |
| | | ATTAATGGTCCGATGTTTTGCAGTCATAATTTTTATAGCAAAAGCCATATGTTTTTATGTAAAGGGATCGTGCCGCTCTACG |
| | | ATGGGGTTTGTTTTAATTGTGGCCAACGACGATTAAAAGATCAAATCTAGCCTTGTCTCTGTACTCTCCCGTCTCCCCCCCC |
| | | ATACACACACTTCTTAAGCGGACTATTTTATATCACAATTAATCACG |
| 471. | HOXB2 | GTCCCCCTAGAGTTTAATTATTCCTGAGATTTCATTGGAAGGAGTCTACCAAACGGAATTTTTC |
| | | TGTGTGAATTTTAAAAGATAACCGAGTGCCCAATATTTTAGAAGAAGAAGAAAGGGAGTGGATTAAACGCTAATTCAGTAAT |
| | | ACCTGAATTTTAGCAAAACACATAAGTCTATGCGACTGAGGGTGGGAGAGGCTCGATTTTTCCAGTAGACGGCCAAGGAGCG |
| | | CGGGGGTCGAAAGGACCGGGAGGAGGAAACAGGTTAGGGAAACTGCAGGTCGATGGCACAGAGCGTACTGGTGAAAAAATCC |
| | | AGCTCTTCCTCGGAAAAAGGGACCGGGCTGTCGAGAGAACCCTGTAGGCTAGGGGAGAGGCCTCCGGATAGCTGGAGACAGG |
| | | AGTCGGCCGCGAAGAAGTTGAGGTCGGGAAGGAAAGGTGAATCCTGGCGCCCCGAGAAGACGTCTTCTGGCAATGGCCCGGG |
| | | CTCCAGCCCGCCGGCCCCGCGCAGCGCGCAGCCGGGACTCGACGCGCCTGCGCCCTCTAAGCGAACGGCTAAAGGCCGGGGG |
| | | TCCGCGCTTAAGGCCCCCGGCACCACCTCCGGCGGGTGACAGCAGGCTTCCCACGCCGCCCGCGAGGCGGAGGGGCCGCCCG |
| | | GGCTGGCCGCGGGTTCCTCGGCAGGGTCGCAGATGTCCTCCAGGGCTCCCGGGCAGGCAGGCTCCCCATCCGGCGGCTCTCG |
| | | GTGCTGCGTCTGCCGCTTGTGCTTCATGCGCCGGTTCTGAAACCAGACTTTGACCTGCCTTTCGGTGAGGTCCAGCAAGGCC |
| | | GCGATCTCGACGCGGCGTGGCCGGCACAGGTACTTATTAAAGTGGAATTCCTTCTCCAGTTCCAGCAGCTGCGTGTTGGTGT |
| | | AAGCCGTGCGCAGCCTGCGCGCCCCGCCGCCACCAGCCTCCGGCAGTCCCAGGCCATCTGCAGGGAAAACAGGCTCGGCGTC |
| | | ATAGCGGGCCGTGTACTCAGCCCAACCTCAGTTCGGACTACCCCATCCTCCAAAACCAGTATCACCTCTCCCCTTCCTCGCC |
| | | ACCCCACCCCCGCCCCCACCCCAAAGCCGCTCGCTTTACTGCTTTTGGGTTTTCTTCTCTCCCTCTCTAGTCTACAGCCCCG |
| | | GCCCGGGTCAGGGCAAAGCATTCAGAGCCGCCCACGGGCCACGCAGGGGGCGGAGAGCGGCTCCCTTCGGCGCCGGAACCTC |
| | | AGATCCCCCTCCCAAGCCCTCCCAGTGGACCCCTCAATTCGGAACTTTCCAACTCAACCGGAGCGAGGGTCAGAAAGATTGT |
| | | TCTTCACCCCTCTTCCTCCCTAGCTCTGAATTCTTCCTCTTCTCCTAGAGTTGGGGCAGAAAGGACCGAATCTTTTCTGCTT |
| | | CTAGGGTCTATCCCAGCAACAACATACTC |
| 472. | HOXB2 | GTCCCCCTAGAGTTTAATTATTCCTGAGATTTCATTGGAAGGAGTCTACCAAACGGAATTTTTC |
| | | TGTGTGAATTTTAAAAGATAACCGAGTGCCCAATATTTTAGAAGAAGAAGAAAGGGAGTGGATTAAACGCTAATTCAGTAAT |
| | | ACCTGAATTTTAGCAAAACACATAAGTCTATGCGACTGAGGGTGGGAGAGGCTCGATTTTTCCAGTAGACGGCCAAGGAGCG |
| | | CGGGGGTCGAAAGGACCGGGAGGAGGAAACAGGTTAGGGAAACTGCAGGTCGATGGCACAGAGCGTACTGGTGAAAAAATCC |
| | | AGCTCTTCCTCGGAAAAAGGGACCGGGCTGTCGAGAGAACCCTGTAGGCTAGGGGAGAGGCCTCCGGATAGCTGGAGACAGG |
| | | AGTCGGCCGCGAAGAAGTTGAGGTCGGGAAGGAAAGGTGAATCCTGGCGCCCCGAGAAGACGTCTTCTGGCAATGGCCCGGG |
| | | CTCCAGCCCGCCGGCCCCGCGCAGCGCGCAGCCGGGACTCGACGCGCCTGCGCCCTCTAAGCGAACGGCTAAAGGCCGGGGG |
| | | TCCGCGCTTAAGGCCCCCGGCACCACCTCCGGCGGGTGACAGCAGGCTTCCCACGCCGCCCGCGAGGCGGAGGGGCCGCCCG |
| | | GGCTGGCCGCGGGTTCCTCGGCAGGGTCGCAGATGTCCTCCAGGGCTCCCGGGCAGGCAGGCTCCCCATCCGGCGGCTCTCG |
| | | GTGCTGCGTCTGCCGCTTGTGCTTCATGCGCCGGTTCTGAAACCAGACTTTGACCTGCCTTTCGGTGAGGTCCAGCAAGGCC |
| | | GCGATCTCGACGCGGCGTGGCCGGCACAGGTACTTATTAAAGTGGAATTCCTTCTCCAGTTCCAGCAGCTGCGTGTTGGTGT |
| | | AAGCCGTGCGCAGCCTGCGCGCCCCGCCGCCACCAGCCTCCGGCAGTCCCAGGCCATCTGCAGGGAAAACAGGCTCGGCGTC |
| | | ATAGCGGGCCGTGTACTCAGCCCAACCTCAGTTCGGACTACCCCATCCTCCAAAACCAGTATCACCTCTCCCCTTCCTCGCC |
| | | ACCCCACCCCCGCCCCCACCCCAAAGCCGCTCGCTTTACTGCTTTTGGGTTTTCTTCTCTCCCTCTCTAGTCTACAGCCCCG |
| | | GCCCGGGTCAGGGCAAAGCATTCAGAGCCGCCCACGGGCCACGCAGGGGGCGGAGAGCGGCTCCCTTCGGCGCCGGAACCTC |
| | | AGATCCCCCTCCCAAGCCCTCCCAGTGGACCCCTCAATTCGGAACTTTCCAACTCAACCGGAGCGAGGGTCAGAAAGATTGT |
| | | TCTTCACCCCTCTTCCTCCCTAGCTCTGAATTCTTCCTCTTCTCCTAGAGTTGGGGCAGAAAGGACCGAATCTTTTCTGCTT |
| | | CTAGGGTCTATCCCAGCAACAACATACTC |
| 473. | HOXB5 | GCTCTGTCTACGAAGCTATGAGGCCCCTCTTAGATACTTCTCCCCCTTAGAAAAATGAATCTAT |
| | | ATTTAGGGTAAAGCCAAGCTCTCCTTGTCCCCCCGATCCCACCCCAAAACGCAGAGAAGGAAAGCCGAGAAGACAAAAAAGA |
| | | GAGAGAGAATTACCATGGCTGATGTGAAGCTTCCTCATCCAGGGGAATATTTGCGGAGTCTGCCCCTCGGGCGCGGCTGTGG |
| | | AGGTGGCCATGGGCTCTGGCTGCGCCCGAGCTAGGCTGGGGCTGCTTAGCTGGCTTGCCGCTTCCTCAGGCTCCGAGGACGC |
| | | GCTGGCCTCGTCTATTTCGGTGAAATTGGCGCTGGAGCTGGCTGAGGTCGCCTGGTCGGAGGGGGACGAAGCAGAGGGCTTG |
| | | GCGCCGTGGCTGTCGCCGTTGGTGCAGGGCAGGGACTCGGGCGAGGACAGGGAGCAGCTCGAAGCCGCTTGCCTGAAGCGGG |
| | | GCTCCTGGGCGGGCGCGGGGAAGGCGCGCGAGCTCTCGCCCACCGCCCCAAAGTGGCTGGAGGAGGCCGAGGAGCGGTTGAC |
| | | GCTGAGGTCCATCCCATTGTAATTGTAGCCGTAAGAGCCGGTGTGCATGGCAGCGGGATCCCTGTAAGAGCCGCTCAGAGAG |
| | | CTGCCACTGCCATAATTTAGCAACTGATAGTCCGGGCCATTTGGATAACGCCCCGAGAAGGAGTTTACAAAGTACGAGCTCA |
| | | TTTGGGTGATTTTGGAGGGCTTGATTTGTGGATCGTGGTCGTTATAACCCTGTGCTTCACGATTTATGATGTATTAATGAAT |
| | | TATAGCGATGCACTGTACTTCGTTTTGCTATGTATGCGGCCAAATATGGGGGGGGGGTTGGAAGGGGGGTGGGGGGCGTTAG |
| | | GGAATCACGTGCTTTTGTTGACCAGTCGTAAATTCTCGCTGATGACCTCAAGAGGTAATTCATGCTCTATGGTTACAAATAA |
| | | TGACGATCCGAGAATCGTTAGGGCCGATTCAATGCGAGCCTCCGAGAGAGGGGGAAAAAAGGAGAAGGGGGAGAAACAGAGA |
| | | GAAGGTTGGCTTTGGCCTCTGAGCAGAGCGCGCCACCTCCCGGCGACCGACGGGAGCGCGGGCGTGAGACCGCCATGGCGGC |
| | | GGGCGGCTGCCTCCCTGCTGGCTCCCTGCAGGCTCCCTGCAGTCGCCGGGAGAGAAAGAAACCCGGCCAAGGCTCAGCCACA |
| | | GGGTTGAGCCTTTTAGAGCAGGGGTTGGGCTTTTTACCCACTTTCTTGTGGGTGGGGTTAGCCTCTCTATTTGGTTCTTTCT |
| | | CCCTACATCATCATTGTCGTTATTATTTTAACTTTGGATGA |
| 474. | HOXD11 | GGGTGGGGTTCGGAACCTCACACCCTCACACCTAGTTCCTAGCTTCTAGGGAGCCTGGAGCCGG |
| | | GGCTTCCCCCCCTCTGCTCGCTGCTTCTCTCCCTTCCCCCTTCCTCCCCCTCTTCCCCCTCCTTCCTCCTCCCCGCCGGCCT |
| | | CGGTCCGCGTACTTAAAGCGGCGCGGGAGGGCGGACGCGGGCGGGCGGCCCGTTCGGGCGGTGGCAGATGCGCCCAGCGGTG |
| | | ACAGCGGCCAGCGGCGCGCAGGTGACCGGCCTGAGGCGCAGCCTGGTCAGGGAGCGCCCGGGGAGAGCTGGCGGCAGAGGGC |
| | | AGCCGATCCGCCCCCAGCGCGCGCGTCTCGGCGCCAGGAGCCGTCCCGGGGCGTGTTGGCGAGCGTTGATATAGATATAAGG |
| | | ACATTTCTCTTCATGGCGTCACGTGACATAATTACCACCAGAATCAATCAAGATGAATTGCACGTCAGCGCCCGGTGGGGAT |
| | | TTTTGCTTAGTTGATCCTGGCCCAAGCCTCTTGTGCAATCGATGGCTCAGGTTGGCTGCGCGGGGAGCGGCCAGAGGCTCGC |
| | | TGGCGCGCACGCCGCGGAGTCATGAACGACTTTGACGAGTGCGGCCAGAGCGCAGCCAGCATGTACCTGCCGGGCTGCGCCT |
| | | ACTATGTGGCCCCGTCTGACTTCGCTAGCAAGCCTTCGTTCCTTTCCCAACCGTCGTCCTGCCAGATGACTTTCCCCTACTC |
| | | TTCCAACCTGGCTCCGCACGTCCAGCCCGTGCGCGAAGTGGCCTTCCGCGACTACGGCCTGGAGCGCGCCAAGTGGCCGTAC |
| | | CGCGGCGGCGGCGGCGGCGGCAGCGCGGGGGGCGGCAGCAGCGGGGGCGGCCCCGGCGGGGGCGGCGGCGGCGCGGGGGGCT |
| | | ACGCTCCCTACTACGCGGCGGCGGCGGCGGCGGCTGCGGCGGCCGCGGCGGCCGAGGAGGCGGCCATGCAACGCGAGCTTCT |
| | | CCCGCCCGCGGGCCGCCGGCCGGACGTGCTCTT |
| 475. | HOXD13 | AAGGGTCTGCTCCAATGCCTCTTACCTGTGTGAAATGCCTTTGCCGGGTACCAGTGCACAAGGT |
| | | AGGGCAAATTAGCTCACTCGGATTTGGGGTCTAGAAGTCGACTAACTGAGGGATTCAGCAACAGGATAAAAAAATGGGCCTG |
| | | TTTTCACATCATTCTGATCATCTCTGTCCTTCGTCTTCATTTTGCTGTGCAACTCGGGGAGCCGAGGAGAGGTGGCAAAAAC |
| | | AGCGGTTGCCGAGACAAGGCGCAGGCCTTGGCGCCCGCCTCAGTCGCAGACAGGGCCTGGGATGGGCCGTCGCGCAATCAAC |
| | | TCGTGGGGGTGGCTGCAGCGCGTACGCCTGGGTCGGGGGGGAGGGCGGGAATGGGAGGTGGACCCTGCAAGGGGCAGGAGAG |
| | | GGGTGGGGGCCGGAGTGGGTGGGTCCAGCCAGGCCTGGGCCGGGAGCCAGGCTCCCCCGCGTTCCTACCCCCACGTGGCCGC |
| | | GCGCAGCCAATGGCACGCCCCCGGCGGGGGCCCTCGGGGCGGGAGGCGGCCCCCCGACCGGCCCAGGCCCCCTCCCAACCTG |
| | | AACTTCGTTTTTATAAACGTCCCGCGATGAGCTAACCTGTTGGAGGGCAGGCGGGCCGGAGGCGGGAGGCTCACAGAGGGAG |
| | | AGAGGGCTAGAGGAAGAGGGCGGGAGCGAGCGAACCAGAGAGAAAGGAGAGGAGGGAGGAGGCGCGCCGCGCCATGGTGTCC |
| | | TGCGCGGGGCCAGGGCCAGGGCCGGGGCCGGGCCAGGCCGGGCCATGAGCCGCGCCGGGAGCTGGGACATGGACGGGCTGCG |
| | | GGCAGACGGCGGGGGCGCCGGTGGCGCCCCGGCCTCTTCCTCCTCCTCATCGGTGGCGGCGGCGGCGGCGTCAGGCCAGTGC |
| | | CGCGGCTTTCTCTCCGCGCCTGTGTTCGCCGGGACGCATTCGGGGCGGGCGGCGGCGGCGGCAGCGGCGGCTGCGGCGGCGG |
| | | CGGCGGCAGCCTCCGGCTTTGCGTACCCCGGGACCTCTGAGCGCACGGGCTCTTCCTCGTCGTCGTCCTCTTCTGCCGTTGT |
| | | AGCGGCGCGCCCGGAGGCTCCCCCAGCCAAAGAGTGCCCAGCACCCACGCCTGCAGCGGCCGCTGCAGCGCCCCCGAGCGCT |
| | | CCAGCGCTGGGCTACGGCTACCACTTCGGCAACGGCTACTACAGCTGCCGTATGTCGCACGGCGTGGGCTTACAGCAGAATG |
| | | CGCTCAAGTCATCGCCGCACGCCTCGCTGGGAGGCTTTCCCGTGGAGAAGTACATGGACGTGTCAGGCCTGGCGAGCAGCAG |
| | | CGTACCGGCCAACGAGGTGCCAGCGCGAGCCAAGGAGGTATCCTTCTACCAGGGCTATACGAGCCCTTACCAGCACGTGCCC |
| | | GGCTATATCGACATGGTGTCCACTTTCGGCTCCGGGGAGCCTCGGCACGAGGCCTACATCTCCATGGAGGGGTACCAGTCCT |
| | | GGACGCTGGCTAACGGGTGGAACAGCCAGGTGTACTGCACCAAGGACCAGCCACAGGGGTCCCACTTTTGGAAATCTTCCTT |
| | | TCCAGGTAGGGGCGATGGAGAAAAGGGACCGACACGAGGGAGGGGGAGAGAGAAGGAGAAAAGAAAGGACTAAGAGTATGCG |
| | | CCC |
| 476. | ID3 | GACAAGTTCCGGAGTGAGCTCGGCTGTCTGATTAGAGGAAAAGAGGGAAGAGTTACGCGAGGCA |
| | | ATCGGGAGCTCCGAGGGTCCCGCAGCATCCTTGCCTGGGTGTTCAGCCCTGTCCCGACTTCGAGGCTTACCTGGATGGGAAG |
| | | GTGGGGGCCATCAGGGGGTCCAGGGGCTGGCTCGGCCAGGACTACCTGCAGGTCGAGAATGTAGTCGATGACGCGCTGTAGG |
| | | ATTTCCACCTGGCTAAGCTGAGTGCCTCTCGGGACTCCGGGTACCAGTTCCCGCAGGCGGGAGTAGCAGTGGTTCATGTCGT |
| | | CCAGCAAGCTCAGCGGCTCCTCAGCTGCCGGGCCCTTCCCTCGGCCCCGGGCGATGGCCAGACTGCGTTCCGACAGGCAGCA |
| | | CACCGCCTCGTAGCAGCCGCGCACCGGGCTCAGCGCCTTCATGCTGGGGAGTGAGTCCAGAGGTGCCCCAAAGAGAAAGAAA |
| | | ACCAAAAGAAGTCCCGCTACAGTGACCTGCAACGCGCGCACGCTCGCCGCGGCGGTCACTTATAGAGCCTGCCTGGAAGGCA |
| | | CGCCTCTTTATTCAAAATGGCCCGCCTCGGCCCTGCCCCCGCCGGCCCTGGGCGTTCACAGCCCGCTTAAATTGCAAACAGG |
| | | CTTCCTCCGGCTGGTCTGACGCCGAAGACCGCGGAGCCGCGGATTCAAAGAATGAGGAAGCGCTGATACCGGGGAGAGGCGG |
| | | GCCTCTCCGCCAGCAAGGATTTAAAAATCACTCAAAACCATTAACTTCCAGAATTTGCTTTTTCCTGGCAGCACCCCAGATC |
| | | TTTGAGCTTCCCTGCCCCCTGCCAGTCCGCCTTTAGCCCAACACTGGTTCGAGCCACAGCTCCTCCGAGGTCATAAATCCCT |
| | | GAACAGCAAAGAAGCTCCCCCCACCCCCCGTTTTTTTTAAGCGAA |
| 477. | IFI27 | AAACTTTGGTTCAAATCCAGATCCACCACTTGTCAGCTGTGGCTCTGCGGTGGACGTATTAATC |
| | | TCTGAGTCTCTTATTTCCCAGTCTGCACAATGGGAAAAACGAGGACATCTGTCCCACAGAATCCTTATACGCCACTACGAAA |
| | | AAGTGACGCACGTCAAAAAAAACCGCTGAGCATGGCGCCCGGCGAGGAGAGCACGTTCGCACACAGTGCCCGCCGGACCCGC |
| | | TGCGCCACGGCAAAAAAACAAAAAACAAACAAAAAAAAACACAAAGAAACACGCGGGGTTCAACAAGGAGAGGGCGAGGGGT |
| | | GCCACGCGAACCGGGCGAGGACACGGAGAGCGCCAGGCAGAGTAGAAGGGCCTCTGTCTCCTCGTGACGCCGGTCCCGCGCG |
| | | GCCCTCTCGCTTTGTCTCAGGCACGAACGCGCGCACGAAACCGAGAAACCGAGAAGCCGAGAAGCCAAGGCCGTCAGGCTCT |
| | | GATGACCGGACAAGGAGCCCAAGGCGCGGGGACCGTGGCACGCAGCTCGGTTGGACGGCTTGGGCCGGCGGCCGCCCTCTCT |
| | | GGACCCGGGAACCCACCGGCCCAGAGCGACCCGCGATAGGAACCCGGGTTCCTGGCCTCAGCCCCTCTCCAGAGTCGGCTCC |
| | | AACCCCGCTCGTTTTGGTACTCACCGTGTGGAGACGCCACCGCAGCTCCGTCAGTCGCGAGTGAAGAACCTCAGAAACCGCC |
| | | GCTGTACCTCAGCTGCAGCAGCAACTGCAGTTCCGGGGCGGGACCTCCACGCACGTACTCGTGCGCGCTGGGGAGGAAGTCC |
| | | CGCCCCTATGGCAAACTCAGCTACCTGATTGGCTGCCTCGCGGACCGCAGCAGTGCCGGCGGGAGAGCTGGCTTGGGGCGCT |
| | | GGCACCTCCTCTTACAGCTTTACTCCTGCCAGCTTGGGAAAAGGCCGGAGAAGGTGAAATTCTGTGTGCTCCCTCCGGCGAG |
| | | AGACTTTGTCAGCTCCCGCACAGTAACGTAAGTTTTCTTGTATTCTTAGTGTAGTTTCGTTACCGGAAAGGGGTCTCGATCC |
| | | AGACCCCAAGAGAGG |
| 478. | IL6ST | AATAGTGTTTATAAGCATTGTGAACTTTCAACAAAGCTGAAACGTTACGATTTCAAGATGAAAA |
| | | AACGGCTTTGGGGAAGGGAAATCCACCTCGAGAATGAAGACAAGTGCGGAGCGAATAGAATTTCAGGGGAGATGAAAAGGGG |
| | | ATTGGGAAAAACGTGGGATCTGGTAACAAAAAGGGGGGTTTGTTCTTGAAGGGCGTTCTGCGGGGCGGGCGTTAAGAGGGGT |
| | | GCGTGCGTGCGCCTGGGTGAAGTTTGTACTCAGGGCTGCGATAATCGAGGTGACAAAGTTGTGGAAGTAGAATTCGGAGGTC |
| | | CGGGAGCAGGAAGGGGAAGGAATGTGGGGATTTCCAGGCTGGGCTGACAAGTTCCGGGCGCTGCTCTCAGGGGGAAGGGAGG |
| | | TCTCCTCGAGTGCCAGATAGCCCGTCCGAAACCACCGACACTCTAACTCCAGCTACGCGACCCAGCACCCGGCCCCTCCTCA |
| | | CCTCAGGCCGCGCCGCCTCGACCCTCCGCGTCTCCACAGCGCACGAACCCCTTGGCGCCAGGCTGGGCCAGACCCGTCGGCC |
| | | TGGCAGGCGCGGCCCCCGGTTCAGCTGCGCCGGGGCGGCCCAGCGCGACTCCGCGGGCCTTTTGGCTGCTCGCCCCGGCTCC |
| | | GGAACACTGTCAGATCCTTCTCCGCAGAGGTAGCGCGCCACCCCAGTCCCGCGGCGGGGCGGGGCGCTCCCCAATCCCGCCC |
| | | AGCTGCGCCCCCGGCCGGCCAACGCTGCGCCCCGCCCCCTACCGGGACCTAGGCGCTGATTGGCCCCTGAGAGACCCTTTGC |
| | | GCCCAGGCATTGGTCCAGTGGCCGCCTGTCGACGAGGGGGCGGGACTCCGAGGGGTAGCGATTCCCGTAACGCGGCTCTTCG |
| | | CGTCCCGGATTCCGGTGGCTGCGTCCGCGTCGCCTCTCCCAGACTAGATCGAAGGGTTCCCCCCACCTCCCCTCTGTGGGCG |
| | | GAAAGAGCGGAATGTTCCATTTTTCTGTGGAATAACGGGGTCATGAACTGGAGCCCGGGTGCTGCCCGCCGCCCGCTGCCCC |
| | | CGCCCTCCCTTCTCGCCAGGGCGCCTGCGGCCTCTCTTCGCGGCCTTCCCCGAGGGACTCAGAGCCGCGAGGGACCGCCTAG |
| | | CTCCCTTGGGGATGGGGAAGGCGTGGGAGCCTCTTGGGGAAAGTGAAGAGCTCGGTGGCCGTGACTCCGCACGAGACTGCGG |
| | | CAGATTGTGGCGGCGGCTTCTCTCCAAGACTTACGTAAACGAGCCTTGCCACAGTACTTTTTTAAATTAGTAAATGAGTTTG |
| | | GGTGCGATTGATCAGTGAGCCCCAGAGGTGGTGGCCCACCAAGCGACTAGTGTTTCTTTGCGGTTTGTACCTCCCCGTTTAT |
| | | GATAAAAAATACCGGGTAAAAACCTATTCATCCCGCCTCCGCCCCCCCCTTTCCCTCAGAAAATGGACCTCAAGGCT |
| 479. | ISG20 | CCCGAGGAGGTAAGGGCCGATGTTCCCAATCTGCAGATGTGGGTGTACTCAGTAAGTCATTCGG |
| | | CCTTTGGTGGTACCAGGCTTACAGGAGCCCACATCCCGACCCAGGTAGAGGAGTGGATTGGAGCCGGGGAACACTGATGGGA |
| | | CCGGGCAGGGACGACAGGAGGTCAGTCTCCCTTCCCTTCTCTTTAAAATTTCCCGCCAGTATGGCCGGCTCGCAGCACGAGT |
| | | TACGCCCTCTTCAGGAGTCCCTCCCCTTCTGCTCTCGCCCGCCTCCCCGCTGCCAGGCAGGCTCGACCAATCAGAAGAGGTC |
| | | TTGGGCGGGCGCTGCCGTCCAGCGAGCCAATAGGCGGGCAGTCGGAGCCGGGCTTGCCCGGGCATGTGGGAGCTGCCGGCTT |
| | | TCCGGACGCCACGTGCAGACCGGAAGAGACACGCGGGGCTTCAGGTGAGATCCAGGACCCTGCACCGGGGCGGCGGGGTCGG |
| | | GCGGGCTGAGGCCGCGTGTCCCGGGTCGCAGGTCCCCGCGCGTTGTTTCCGCCCCGGCCTGCGGCGGGGACGCCTGAACTGG |
| | | GCTGACCCTGCCCTACCGGCCCGCAGAAAAGCAGGCCGGGCGCCGCCTGTCTCCAGCCCTCGCTCTGCTCGGGGGTCCAGGG |
| | | CCAGAGGGGGCGGCGCTGGTCGTTGGGACTCGGCCCAGCTGCCGCGGCCAGCAAGTGACCAACGCCCGAGTCCTTCCCGGTG |
| | | AGATCAGGCCTCCCACGCGTCACGCCTGGGAGAACGCCAGCCGGCGGGGAGGTCGTGCCAGGATAGCAGACTTTAGTTGTTG |
| | | TCGTTGTTCCGTGGACTTTTTAGCAGCACAGGGGACCTTATCGAGCCAGGTTTTTTCAGGTTTGGGCGATTATCAGAATTGT |
| | | GGCTTGAGGGTAGGGGTCGTCCGCGCTCTTTTTAAAAATCTAGATTCTCAGGCCCCTTTCCCCAAAATAGCAATTAGGACGG |
| | | TGTGGGAGT |
| 480. | KIAA0476 | CTCATCCACTGAGGGCGAATGGGAGCCCTATGCCCAAGGCAGCAGGCAGAGGCCATTAGGTGGG |
| | | ACCTTCTGAGGGGCAAGTGGGTGGCACCCATCCGCAGGACCTGCGCACCGGGGCCTCCCCTCACCAAGCCAGGGAAGAGTCC |
| | | TGCAGAAAGCAGCCCTTTCTCCCTAGGGAAGACAACGGGCACAGGGCCCACTCCGGGTCCCCACGCCCGCGTGCCGGGTGAC |
| | | GGGAAGTTCGGGTTGGGGCAGAGGGCGCGCCGAGGGGGAGGAGGCTGAGGAAGGAGCAGGAGGGCAGAGAAGCAGGCGCGCC |
| | | GGCGGCCGAGGACGTGACGGCAGCAGCGCCGGCCGCCGCGTGACCCAAGCGGGAGGGAGGGCGGCTCCGACTCCGGCACCCA |
| | | CGGGAGCAGAAGGGGGTGCAGGAGGCCGGGCACGGCGAGCTAATTGCGGGAGGTGCCCTCCCCTCCACTTTCACTTCCCCAG |
| | | GAGAGAGGAACCGGAACCAGATGTGCAGTGAGGGACAGCAGCTGGGGGCCATCCCCCACCCCGCCTGCCGCCCGCGCTCCCT |
| | | CCTGCCCGTCCCCGCCTGCCGCCCAGCCCGGTCCAGCCCCTACCTGCCTGTCCCGCGCTTCCCGGCCGGCCGGCCCGCTGGC |
| | | GGGTGGCTCGGAGGGCGAGCTGGCGGGCCGGCGGGCGGCGGGGCTACCCGGCCCCAGACATGGCGCACCCGACTTGGGCGCC |
| | | GCTCCCGCCCGGGCAGTGAGTGAAGGAAGAAGAGGCGGCCGAGGACCGCAGAGCGCGGGGCGCAGTGGAAGGAGATCCGGGC |
| | | GGTCCCCGCGTCCCCGCCGCGCTGCGCCACGCGGGGACTGTGCTGCCGCGCTGCTCGCTCGGCCGGCTCGGTCCTCCCAGCT |
| | | CCCGCGGCGCCGGGGACCCAGCGTTCCTCCGCGCGCCCCGCTTTCTCTACTCCCCCAACCCCCGCTCCGGGCCGCGGGCGCC |
| | | GCCGCTACCCCCACCCCTCCTCCTCGGCCGGCGGCCGTGACCCTGGCAAGCGTCTGCCCAAAGCCCAGCCGCCTGCAGCCCG |
| | | ACCCCTGGCGGCTGGGAGGGCCCCCTGCACCCAGGTAGCTTCCCACCATCCAGCCCGTGCCTCAGGACACTGACCCCAGGCG |
| | | ACCCAGAGGGCCGCGGCGCGCTCCCTTCCCGCCTGGGTCCTGCAGTCAGATGTGGCAAGCATGATGTGGGCGCAGGTGAAGT |
| | | CCCGAAGGTCTAGTCTACGGGGACCCAGGTGGCTCCCCCTACTTCCAGGTTTAGGGCACAGGGCATTCCCTTTGGACCAGAA |
| | | CCAGAAGCTCTCAGTTCTCTTTTTTGAAAATGACAAGAGCTTTGGAAAGAGGAAGAGAGGCCAGGAC |
| 481. | KIAA0830 | GTTATACTGTCTGCCTAGTTTAGGAACTTCCTTTAACAGTATTTCTTTACTCTCTGAGTTTCTG |
| | | GTGAGCTAAAAAAACATAACAAAAAACAAAAAACTCCATCAAAATTGTATGCGTGTGCTGGTCGTTGAGTACTTCGGTGGAG |
| | | AGTGGGAGACGCATCTTCATGAAATTTCAGAGGGGAACTCTGCCCATCCACTCCGAACCAGCCGCAGCTCCACGAGCATCTC |
| | | ACGCCACCCCTCTGCATCACCAGGGCGGCCGTGGGGCGCGCGCGATCAGCAGGCTTCTTGGCGCCCAGTCCATGCGACTATC |
| | | TCTTCCCAGCCCCGGGACTAGGCCCGCCCCACTCGGATTCTCCACTCTTGGTAGCGCGCGTCCCCCGGACTGCTGAGACCAG |
| | | GCGGCGCGGAGGCAGGTGACCCGCGTCTCCAGTCCCGGGCGCAGCCCAGGGTACGTTGTCAGCATCGCGGAGCGCGGCCCTG |
| | | GGCCTCTGCAGCCATCTTCAGGGAGGGAGGCGCGGCCTCCCGACGCGGACCCGCCCCCGCCGCCCGGGCCGCCCCGCCCGGC |
| | | TCCGCAGAGCGCCGCGCCTAGGTTGCGACTTCCCTTCCCTACCCTGCTCGGCTGCGTAGTGCGCTCCCCGCCCAGCCTGCAG |
| | | AGCTCGCGCCGCGGCAGCCCAGCCGCTCGGCCCCGCCGCGCTCGCAGAGGCCGCCATGGGCACCGCGCGCTGGCTCGCGCTG |
| | | GGCAGCCTCTTCGCCCTGGCTGGGCTGCTGGAAGGCCGGCTCGTGGGCGAGGAGGAAGCCGGCTTTGGCGAATGTGACAAGT |
| | | TCTTCTACGCCGGGACCCCGCCTGCGGGGCTGGCGGCCGATTCCCACGTGAAGATCTGTCAGCGCGCGGAGGGTGCTGAGCG |
| | | CTTCGCCACCCTCTACAGCACCCGGGACCGCATCCCCGTGTACTCCGCGTTCCGCGCCCCGCGCCCTGCGCCCGGCGGCGCC |
| | | GAGCAGCGATGGCTGGTGGAGCCGCAGGTAAGCGAAGTGGTTCCCGAGCCGGGCTGCGGGCGCCGGAGACCGTGCCGCTGGA |
| | | CATGCCCCCAGTTGCAGCTACCGCGAGGGGCGGGCCGGGAACAGCAATCCCTACGCCTTCGGTGCCTTGGGCCAAGAAACCC |
| | | GGGTTCCAGGTCCACCCTGTTGCGTCCCCGGAGTTAGCCTGCCTCGCCCCTGTCGAGCTACAATAAGAGCCTGGCGATCTGC |
| | | GAGCCTTCACTTCATGAGCTGCAGGGCGGGAGGTCCCCTGTGGGAGGAGGTTCTCCCTGCATTTCTGAGCAGACATCCGCAG |
| | | TGTTTCATTCCTGAATGGTGAAGAGCTTGATACAGAGACCCTGGAGACCGTTCAGATCCGCAGAAAGCCTAGGCTGGGCGCT |
| | | CACAGGCCTTCGTGCAACTTATTGCCATAGCACTCAT |
| 482. | KIAA1447 | GGTATTTTACCGGACTTCCCAGAATCCGGATCGGGGAAGGCACCCTCTGGGGGCTGGGGGAACC |
| | | AGGAGGCCCGTGGGGTAGGCAGGGTCCGGGGAGGGGCAGGTGGAGGCAGCTTTGTGGGCCCAGCTGGGGCTGACTCTGCTGG |
| | | GCTTTTGCCCTCAGGTAAAGCCGAACTCCTAACCTCAGGTGCCAAATCCCCCACGGGGGCCTCCGACCACTTCCTGGGCCGC |
| | | CGTGGCAGCCCCTTGCTGAGCTGGTCCGCGGTGGCGCAGACCAAGCGGAAGGCGGTGGCAGCGGCCAGCAAGGGGCCGGGGG |
| | | TGCTGCAGAACCTCTTCCAGCTCAACGGCAGCAGCAAGAAGCTGCGGGCCCGCGAGGCCCTGTTCCCCGTGCACAGCGTGGC |
| | | CACACCCATATTTGGCAACGGCTTCCGCGCCGACTCCTTCAGCAGCCTGGCCAGCTCCTACGCGCCCTTCGTCGGGGGGACC |
| | | GGGCCGGGCCTCCCCAGGGGAGCCCACAAGCTGCTGCGGGCTAAGAAGGCCGAGAGGGTGGAGGCCGAGAAGGGTGGGCGGC |
| | | GGCGGGCGGGCGGTGAGTTCCTGGTCAAGCTGGACCACGAGGGTGTGACCTCCCCCAAGAACAAGACCTGCAAGGCGTTGCT |
| | | CATGGGGGACAAGGACTTCAGCCCCAAGCTCGGGCGGCCCCTGCCCAGCCCCAGCTATGTGCACCCGGCCCTTGTGGGCAAG |
| | | GACAAGAAGGGGCGGGCACCCATCCCCCCGCTGCCCATGGGGCTGGCGCTGCGCAAGTACGCGGGCCAGGCAGAGTTCCCGC |
| | | TGCCCTACGACAGCGACTGCCACAGCTCCTTCTCGGACGAGGACGAGGACGGGCCGGGGCTGGCGGCCGGCGTGCCCTCCCG |
| | | CTTCCTCGCCCGCCTGTCCGTGTCCTCTTCCTCCTCTGGCTCGTCCACCTCCTCCTCCTCAGGCTCCGTGTCCACCTCCAGC |
| | | CTCTGCTCCTCCGACAACGAGGACTCGTCCTACAGCTCAGACGACGAGGACCCGGCTCTGCTGCTGCAGACCTGCCTCACCC |
| | | ACCCCGTGCCCACCCTCCTGGCCCAGCCCGAGGCCCTGCGCTCCAAGGGCAGCGGCCCTCACGCGCATGCCCAGCGCTGCTT |
| | | CCTGTCCAGGGCCACGGTGGCTGGCACCGGTGCGGGCTCAGGCCCCAGCAGCAGCAGCAAATCCAAGCTCAAGCGCAAAGAG |
| | | GCCCTGAGCTTCTCCAAAGCCAAAGAGCTCTCCCGGAGGCAGCGGCCGCCCTCCGTGGAAAACCGGCCAAAGATCTCAGCCT |
| | | TCCTGCCCGCCCGGCAGCTCTGGAAGTGGTCGGGGAATCCCACACAGGTAGGTCCAGCGGGAGGCGGGAGGAGCTCCTGGTT |
| | | CCCAAGGAAACCGGGGCGGGCTCATGCGCCCCTGCTGCCCTTCCCTCTCCTTTTTCATCTTCCTACTTGATTTCAAGTTAAA |
| | | AAATGTGGAAAACTCAAGGGAAGAACAAAGACCCATCCATGACCCAGTGAGGCAGCCGCTTCCGCTGGCCCCTCGCTGGAGC |
| | | CCGTGGCCTGCGTGACAGTAGAAATGGCTTCATGTCGGGCCGGGCCTGAGCTGCACTCCGCATG |
| 483. | KRT13 | GCGCGGCCCCTGGCCTAGACCTCTGGGCACTCTACAGCCGGGTCCCATCTCCTCCCACTCTTCA |
| | | GGCCTCGCTGACCCGCGTCCCTTCTTCGTCCCGGGGCTCCAACCTTGCCCCCGCCACCTCCAGAGGCCCCACCCACTTATCT |
| | | GCCCTGCCCAGTTCTCCTCCCCTTTCCACCACCCCGGCCTCTGCATCCAGCCCCGAGGTCCTGCCCCGCACGTCTCCTGTCC |
| | | CCCTTTACTCGGCCCCACCTGTGGACCTTCCCACGTCCTAACGGGCTCCTGCCCGCCGCCCCGCCTGGAACCTCGCCCGGCC |
| | | CGCGGGGCTGGGTTTCCGCGGCAGGTGCACTGCACTTCCCGCGGCCGGGCCTCCGCCCACCTTGTCCCGCAGGTCCTGGATG |
| | | GTCGTGTAGTAGTGGCTGTAGTCGCGGGAGGGCCCAGGCCCCTGCTTCTGGTACCAGTCGCGGATCTTCACCTCTAGCTCGC |
| | | CGTTGGCCGCCTCCAGGGCGCGCACCTTGTCCAGGTAGGAGGCCAGGCGGTCGTTGAGGTTCTGCATGGTTAGCTTCTCGTT |
| | | GCCCGCCAGCAGCCCGTCGGACGCGGTCAGGACGCCGCCGTAGCCGCCGCCGTAGGCCCCCGAGGAGGACGAGGACACAAAG |
| | | CGGGCGGAGGACACGGATACGCCGCGGCCGCCGGAGCCCCCGTGAATGCTGGGCGCGCGAAAGGCGACCCCCGGCCCAAAAC |
| | | GCACGGAGCCGCCGCCCAGGCCTCCGAAGGACGACGTGGCCGACGACTGGCGATAGCTGTAGGAAGTCATGGCGAGGCGGAG |
| | | CACGGACGGAGCAACCCTGGTCTCAGAAGCTGCGATTCGCGGGAGGAGCGGCGAGGCCCTCACCTGGCGCCTTTTATGCCCG |
| | | CGGCCGGTGGAGGGGGGAAGGGAGGAATGGTGTCAGGGGCGGATATCTGAGCCCTGAGGAATTTGCAGGCTCCTGAGAGCAA |
| | | ATATGGGCTCTCTCCCCATTGGTCAATTCCCTCCCCTCCCAGAGACCAGAGGCCCCTGCCCTCCAGAGGTGCCCCGCCCCGG |
| | | TCCGCGCAGAAGCTCCGACCCGCACTCCCCCACTCTCTCTCT |
| 484. | LAD1 | ACCCCCCGCCCCTCCCGGCTCCCTCCGGCGCTCACCTGGACAGCGCGGACCAGTCCTTCCTGC |
| | | TGACAGCCATGCTGCAGGAGCCCCGCGTGGCCGCCCGCGCCCCGCCGGCCGCCGCCTCACCTGGCGCCCCTCCCCTTCCAGC |
| | | CCAGGTGGGATCACATGGCCGCTTATCTCACCCCCGCGCGCCCCGGCCGCGCTCCGATTTGCCGACCCGGGAATGGCCTCTG |
| | | GGATGTGGGCGTCCCAGACAAAGCCGCATTGATTGCAGCCCAGGCCGGCCCGGCCGCGAGGCCGCAAGCACTGGGTGGACCG |
| | | TCGGACGGATCGCGGGACCGAACGACGGACGCCCGGGGCCGGCGTGGGGCTGGTCGGCTGCCCGGGGCGAGGGCGGGAGACT |
| | | TCCTGCTCAGAACCAGTCCGACCAGCTTGGCGCTGGGACGCCTCCCCGCGCTGTGCCGCGGCCCGGGGTGCCGAGGAGCTGG |
| | | GCTGGGGGCGGGGACCCCTGCCGGGAACCCGACTTAGCCCGGAGACGCTCAAGAGCAAGAAGCCGAGCGGAAAAGTTAGCGC |
| | | AGGTACTGTGATCTCGGGTCTAGAGCGCCCGCGCCTTCGGGGCCCCTTCGCAGGGAGCGCCCTCCCCACTCCCTGGCTTCGG |
| | | AACCCACCCGGGCCCACCGTCCCAGTAGGCCTCGCTGAGATGAATGAGGCATTGCTTTCTGAGCTGCACACAAAACATGCCC |
| | | CTGGTAGACCCAGCCCCACCCAAAGGGAGAATCTTAGACTCAATTCAGCAAACATTTGTGAAAGCACAAATATGTCAAAGGC |
| | | ACTGTCCTTGCTCCCTGATACTGGACTTTGGGCTAGAGTGGCAGGGTGTATAATAAGGGCAGAGGAAGCT |
| 485. | LAMB3 | TTCCCCCTTGCCTTCTCCTCTCTGATCCATTGCCACACACGTGGGAAGGTGACAACCCTTCCGA |
| | | ATAAAAATGAAAGCTTTCTTCTTTAGATGGAACCCCCAAATTCCCTCATTATTTATAATGTCAGGCTGTCCTGGACAAGGGA |
| | | AGCTGTGCACCCGCTGACACCAGTAAGAAGGTTGCCGCCATGTCAGAGATGTCCGCGGACACCTCCCTGGGCTCCGGGTCCT |
| | | CCCCTGCGCTCGCCTGGAGTGGGACCTTCGCGTGCACACTGGCCTTCCCACGCGCCCCGCTGCGATGGCACCCGCGCCGGGC |
| | | CCCCTAGCTCACACAGTCGGAGCGTGCTCAGCGCGTGGCCACCTCCTGCCAGGTCCCAGCCGGGTTCCACCCCCTCCTTTTC |
| | | CCCTCCTCTTCTTCCTCCCCCTCCGAGTTCCCCTGGCTCTGACCGCGCTGGCCTGGGCCGGAGAGCCCAGGAGGCGTGTCTC |
| | | AGAGAAAAGATATAAGCGGCCCCCGGACGCTAAAGCGGTGCCAGCGGCGGAGTCTCCAACTGGGAGAGCTGCAGCTGCCGAG |
| | | AGGAGGAGAACGCTGAGGTCGGTCGGACCAACGGACGCGCTGACCGCTGCCAACTGCAGCTCGCGCTGCCTCCTGCTCGCGC |
| | | CGTGCCACTAAGGTAGTCCGCCTTTCTATGAGCCCTCCCCAAGATTAGCTGGGTGCGGGGTGGTGGGAGCCGTTCTTTGGTG |
| | | GCTGAAGCCCCTCTCCTGCTGCTCCTCCTGCAGGTCATTCCCGCCTCCGAGAGCCCAGAGCCGAGATGGAAACGGTCCAGGA |
| | | GCTGATCCCCCTGGCCAAGGAGATGATGGCCCAGAAGCGCAAGGGGAAGATGGTGAAGCTGTACGTGCTGGGCAGCGTGCTG |
| | | GCCCTCTTCGGCGTGGTGCTCGGCCTGATGGAGACTGTGTGCAGCCCCTTCACGGCCGCCAGACGTCTGCGGGACCAGGAGG |
| | | CAGCCGTGGCGGAGCTGCAGGCCGCCCTGGAGCGACAGGCTCTCCAGAAGCAAGCCCTGCAGGAGAAAGGCAAGCAGCAGGA |
| | | CACGGTCCTCGGCGGCCGGGCCCTGTCCAACCGGCAGCACGCCTCCTAGGAACTGTGGGAGACCAGCGGAGTGGGAGGGAGA |
| | | CGCAGTAGACAGAGACAGACCGAGAGAGGAATGGAGAGACAGAGGGGGCGCGCGCACAGGAGCCTGACTCCGCTGGGAGAGT |
| | | GCAGGAGCACGTGCTGTTTTTTATTTGGACTTAACTTCAGAGAAACCGCTGACATCTAGAACTGACCTACCACAAGCATCCA |
| | | CCAAAGGAGTTTGGGATTGAGTTTTGCTGCTGTGCAGCACTGCATTGTCATGACATTTCCAACACTGTGTGAATTATCTAAA |
| | | TGCGTCTA |
| 486. | LCN2 | AAACATAAATAAACAAACCAAAAAAAAAAAAAAAAAAAAAACCACGGTAGAAGAACGATT TTA |
| | | TGAGAACTCGAACAATTCCAGGTAGTAGGCATCAGGAGTGCCCCTATTTTACACAAAAGAATACAGAGGGTTCGGAGACTTC |
| | | AGACCAAGTCACTCGCCCAAGGTCACAAGCAGGCAGAGGCAACTCCTGACCCTGCGGGTTCCCAGGAGTCCCGAGTTCGGCG |
| | | CTGATCAGCATCCCCATCCCCGGCCGGGCCAGGCCTTACCTGCGCGGCTGAGCGCTCTGCGTGGAGGTCCTGGGCGCGCAGG |
| | | TGCCACCGCGCCGTGTGGTACAGCCCCAGGGCTCCCCCCAGGGCCAGCGCCGCAGCTCCCAGCAGCCGCGGGCTCCCCTTAC |
| | | GAGCTGCAGCCACGGGGCTCGGGCCGCCCGCCGCCCCCGCGAAGCCAGCCCGGCTCTGCGTGGGTAGCAGCGGCTGGGGGCG |
| | | GCCTCCCAGCCTCCAGGCCAAGGCGCACCCACCAGGCCACAGCGCCCGCACCACCCGCGCAGCCGGGTCCATGTTCGCTCCG |
| | | CCGGCGCCGCGGGCGGGCGCGCGAAACGAAGACGCCGAGGCACGCGCGGCGTTTAAAGGGCCAGGACTCTGGCGCCCCGCGG |
| | | GTTGGCCGGGGTGAGGGCGACGCTAAGGGAACCCTCAGCGCTCTCGGGACTGGGCGTGTGCCCGGCGCCCAAGTTCGAAACG |
| | | CCCGCCAGAGCCGCAGAGGCCCGCTCGGGAACGTTTGCAGACCGTTCCATGCTTCCGCCTCCAAAGGCTCTTGGGTAGTGAC |
| | | CCGGCCGACTGCAGGGGGGCGAACGTCTCCTCGCCCCACCTTGACCGCCTAAAGGGCGGCGTGGCCGTCTGTAACACGTACT |
| | | TGCCCACCTCTCTTACGGGCGCCCATTCAAAACAACTCCAGCCCTTCCTAGAGTCAGTACAGGCGCCGCGACTTCCGGCCAC |
| | | TGAAAGCCCCGGAAACGACCCGACCCGGCACCAGGTGTTGCGGTTCTCTCTGCCCTCCCGCCTCATTGTCCGCTTCCTACTG |
| | | TGACCCGAACCATAAAATTTTCCCGAACCCGAAGGGGCCTTGGAAACCACGTGTCTCTGATTTCGCCCACGCGAAGCGGCCG |
| | | GCCCGCCGACCCCGAGATTTCCAGGGAGAGGATGGCAAAGTGGGGTGCCAGGCTGCTCGGTCCTTCTGCTGGGCTCCCCCGC |
| | | CCTAGCTGAAGCCCGGAGCCTCCACCGCATAGGCGTCTCCGCGGACAGCCCCGGGATGGCCCCGCCCGGCGCCCGAGAGGGG |
| | | GCGGGACTCGCAGAGTGGGCGGGGAGAGGGGCGGGGCGAGGGGGGAGCTCTGAGTCCCGGCTCTGCTGCTCCGCCGCGTCCC |
| | | ACTCTTCTCGCTCCGCTTTCGCCCCGCATCTTCCACCTTCCTTCCAGTCCTTGCGGCGACCGAGCCCGCAGTGTCAGTCCCC |
| | | AGCGGGGACCTGAGTATGCCGGTGAAGACGAGAGCGGAAGGCGAGGACGACGGCTTCGGGGAAGCGGGTGACCCGAGGAGAC |
| | | TGCTGGAGCGACCTTGGCGTTTCCGGGGGTGCCTTCCCGGGAAGGGGAATCGGGATGTTGGCTTCGAAGGGACCGAAGGGCC |
| | | CACCTCGACCCGCCCCGAGTGGGTTTGGAGTTGTAGGTGCTGTCTCGGATGCAGGGCGAGCACCCGCGAGCGGGTAACCAGT |
| | | CCCGTGAGAGCTGCAGGTCCCCAGCCCCGTTTTACAGATAGGGAAACTGAGGCAGCTGCTGGGACACTCGCCCATATGGGTT |
| | | TTGCTCCACCCACTTCCTTCAGCCACTTTACAGACCAGGAGTTGAGGGACTGCTCGAGCTTAGAGTGTTTAGGGGTGGTTGA |
| | | GGGTGACCCCCATGTTCTGTGCTCTA |
| 487. | LGMN | CCCGTTTTCTTGTCGTTACAACTCTGGTAACGCCCTGAAATTGTCTTGTCCATTCACTGTTACG |
| | | TAATATCTGTCTCTGCCACTAGAAAGTAAGCGCCATGCAATCCGGATCATGTTTTCTTATTTCCACCTTCTGCCGTGGACCT |
| | | CTGGCATTCTCGTCTGTAAAACTTAGGGGAGATGGTGAGGAAAGGGAGAAACACGAGGTCTAGCCGGTCCACCCGGGTCTCA |
| | | AGCCCCGCCCAACTCAGGCCTCGCGTTCCTTCCAGGCTGGGCACCGCCTTTGGTCACCCACGTGGCCCGCAGTAAGGCCCCG |
| | | CCCTTTGTGGGTGTGGCCTTTCACGGCGTGCGGGCCGTCCACCGGCGGCGAGCCACGTCACACGCACGCAGGCCATGCGCCG |
| | | CGCCTGTTCTCGCCGCGGGCGGAAAAGGGGCGTGGCCATGCGCGAGGACCCCGTAGCCGCCAGCGCCACCGCCTGAGTCGCC |
| | | TGCAGGTGGCTGTTCGCGTCTCCCTGCCGTGCAGGCAGCTCGAAAGCCACTCTGTTGTGGGGTCCTCGCTTCCCTTCCACGT |
| | | CTCCCGGAAGCGGTAGCAGGGCCCCGGCGGGTCGGGGAGGAGGTTTACTCAGCTTGGGCCCCCTCCGGGCCAGCCGCCGAGG |
| | | GGGCGCGGCCCAGGACGGCGGCTAGGCCGTAGTGCAGCCTCTCCGGAGTCCTCAGGTGAGGCGGAGGCGAGGGCCCACTGGA |
| | | CGAGACGGGGGTGTTAACGGTCCTAGGAGCGGGGGCGCAGTCCCTTCAGTGGAGACCCTGGTCTCTCCGTTTTCTTTCGGGT |
| | | CGCGCTCATCTTCGCCCTGTTAGGGGCTGAGGAGGGTAACTGGAGAGACTCCTTGGGCTTCAGCCGACGTCGTTTACTGCCC |
| | | TCCTCTGTCTTCCTTTCTCCTGACCCCTGCCTGGCCTGGCCGTTCTTCCCGCACGGAACGAGATGGGTGGGGAGCAGAGTGG |
| | | GGGTCTCTGCTCGCTTACCTGAGATTCCCCAAACTGACAGACCCCAGGGC |
| 488. | LOC114990 | CCGGGCTGGTCATGAGGACTCAGCCCTTACCAGCCCCACTCCCGCCATCCTGCCTCTCCAGGGC |
| | | CGCAGAGGCCCTGGGGTGGACTTCTGGCATTTGTCACCTGCCTGGTGCCTCATAGGCATCTGGGCTGTGACGCTTAGGATTC |
| | | CTAAATAGTCTCTCGCTTTTTACACAAAAAGCAGAAACCTGCCCAGTGTCATCCGGCAAACCCACGACGGGCAAGGTCTCCC |
| | | CGCGCAGGTCAGGTGGCCGCAGGGTGAGGGCCAGGGCAACCCGGCCCACGAGCCCCTCGGTGGGGAAGGGGCGTGTCTCCAC |
| | | GCGGCTCCGCCCCGGCCAATGGGGAGAGGCCCCGCCCCTGCCGCGGCGGGCCCGCCCCCCGGGACTCTTAAGGCGCGACCTG |
| | | CCTCCAGCGAGCCGACTCCGGAGCCCGAGCCCGGGGCGGGTGGACGCGGACTCGAACGCAGTTGCTTCGGGACCCAGGACCC |
| | | CCTCGGGCCCGACCCGCCAGGAAAGACTGAGGCCGCGGCCTGCCCCGCCCGGCTCCCTGCGCCGCCGCCGCCTCCCGGTGAG |
| | | TTTCTGTGGGGCTGGGGGGCTGCGGGCGGGGAAGGGGGCGCCACGGCCAGGCTGTGCACACGCGCGGGGACCGCCGCCGCGC |
| | | GCACACACGCACTCTCGGGCGGGACCGCGGCCCCAGCCCCACACGGTCACCTGGGCCGCCCGGCGGGCAGGCCCTGTACGCT |
| | | CCCTCCCCCGCCGTCCTGGGGCGCCGACAGCCCCGCGGCCGGCCAGGGTGCGCGCCTGAGTGTGCGGTGAGCCCGCGAGGCT |
| | | GCAAGCAGACAAAATAAACACCCCGTCCCATGCTTCCTCCTCCCACGGCTGGGCCTCCGGCATGACCAGTTTTATGAATCAA |
| | | ACCCCTTTGAGGGGGATAGCCAAGCCACAGGCTTGGGTCACTTTTCCCTCCTGGGGTCCGGCAGTAGGTGCTGCTGTGCAGA |
| | | AGGGCTGCCCGGGGTCCTCTGGACCCTTGTC |
| 489. | LOC55971 | GAGGGGATGGGGAGGGACTGGGAGGAAGGAGAGGGCCAGGGGAGGAAGAAGAGGGGCTGTGGAG |
| | | GGCCAGGGGAGGAGTGGGAGAGACCGGGAGAGGTGGAAGGACGCGGGGGAGGGGAGCTGGAGGGAGGGAAAGTACCTTCCCG |
| | | CGTAAAGTCCCCTTCTGAACCCCGAGGTGGAAAGCCCTGACCTCCCTGAGCCCCGGGCCCTGGGCTTACCCGGTAGGTGCTC |
| | | TCCGTGAGCCGGTTCACCTCCTCGGGCCCCCGGGACATGGCTGCGGCCGCCGGGCGAGCAAGCGCGGGAGGACGCGGCTGGG |
| | | CCTCGTGGCCGCCGGACTCCGGGCAGCGGGAGGGCCGGGGCGCGACTAAGGGGCTCTGGAGGGTCGGCCGCCGCCGCAGCCG |
| | | TCGGCCCGAGAGTGCCCGCGCGCGTCTCCGCTGCGAAAATGTCAAAACTTGCGGCAGCGCCGCCCTGGCCTTCTTCGAGGAG |
| | | CAGAGGAGAAGCGGCCGGACGCCGCCAGAGGAAGCTGGGCGGGCCGGGCGGCCCCACCTGAGGCGGGCGGGGCAGACGGGGG |
| | | CGGGGCCGCAGGGCCGGAGGGGCGTGGCGTACCCACCTGAGGCGGCCTGGTGAGCGGGGCCCGGCCGGGGTGGGCGGGGAGG |
| | | AGGCGGGGCCAGGCAGCTGGAATGGGCGTGGCCAGAGGGAAGCGGGCCCCCCGAGTGGGCGGGGTCACCCCACCTGTGGCGG |
| | | ACAG |
| 490. | LOC57228 | TCCCCTTTTTCTCTTGTCCTCTTCTGGTTTCCCTGAATATCTCCTCAGGCTCCAGGTACCAACA |
| | | GCTCTCCACAGGAGCAGGTCTGAGGCCGCATGCAGTTTGACGGGGTGTTACCTTCATACGCTCTCTCCAGGTCCTTTCAATG |
| | | GGCCACCAAATTTACTTAGATCTCTCAAGGCCTGAGAAAGTCAGGCAAAGGTTCGCAAATCAGCAGCGAACCCAGTTTCGCC |
| | | AAAGCGCGGTAACAGGTACCCTAGGGTTCCACAGCTAGGAGTTCTGGTGACGCCATCCACCAGGTGTCAGGAGAGGGCCGGG |
| | | AAGGTCAGGACCCGCGCCCAGGCTCCAGAGCGGAGTGGGCGCAGCGCAGTAGCCGGGCCGGGACCGGGAGGCGACGGCCGTG |
| | | GGCCGCCTTCTGGCTCGCCTCGGACGCTGGGACGCCGGACTGGCCGCCCCCTCCGGGCCTGCTTTCGCCGTCCAGGGCGCGG |
| | | TCCGCGCACAGCCTGCCAAGCCTCACTGGGCCCCAGCCCGCGCCCCGCGCCCCGGAGATCCTCGACGCCCCTGCGAACGCGG |
| | | GGTGGACGCGACGGGCTGGGTTGGTCCTCGCCCCGCGGCCCCGCCCCCGTCCCCGCCCCCGCCCCGAGCCCCCCGCGTTACC |
| | | AGCACCCCGCGGAGCGCGCTGCGCTGGGGGCGGCCGGTGCGTCGGGGAGCGGCGTTCCCAGCCGCCGCGACCCTCTGCCCCA |
| | | GCGGAGCGCTGAGCTTCGGCCGCTCCGGGTTTCGGTTCCTGCCACGGCCGAGGTGGCTGCGGCGAATGTGGGCGACCCGGCT |
| | | CTCCGGCGCCCCCGCCCTTCCCTCGTGCTCACCTTTCCAGTCGGACGGGCTGCTGGTGAAGGGCCGGCGCCGCTCCGCGCGT |
| | | CCTTTTGAACTCAACGGGGGCGGGCACCGCGGAGTCGCGGAGGCCAGCAGAGGCCGAACGAGGACCCCGAGCGGAGGAAGCC |
| | | GCGGGTGGCGCGCGGGGTTGGCGCAGAGGCCGGAGGGGGTGGGGGGCAGGCCGACGGGGTGGGACAGGAAAAGCGGAGAGAA |
| | | ACCGCCCTCTGCAGGTCCCCTTGGCTCCCCCGGGAGGAAAGGCAGCCTGCCCTTCTCCGATTGTCACTTTACTCTCCATCCG |
| | | GAGCCGCTTCCTTTCTCGCCGCGAGGCTCGGGGTTGGGGGGGGACCAGATTGGAGCCGCGGGCTAACTGGGATCCGTCCCAT |
| | | TTCCCTGGGCTTGACGTTCTCTGAATTTTTAGCTAATGTGGAAAGTTACATTTATTTGCATTTGTTTATCGCTTGCTCACAT |
| | | AGGTCTGTGTCCTGAAGCTTGGCAGATGAGCGAACTTAGCCAGCACACCCCCGGCCGTGAAGCAGGGAGGTGAAGCGGGGAG |
| | | AGCAACGAGCCCCACCCGGGTCTTGCCAGC |
| 491. | LRP6 | CCTCCCCACGACCAGGCCTCTCCCCGCTCCTCTCCCCTTCTCCCTTCCTCCGTCCCTCCCCTCC |
| | | CCCTAGACACATACAACAAGGCCACCTCCCCCCGAACCCCACCAACTTTCCAGTGCCCCCACTCTTCCCACCTCTCAGGAGC |
| | | ACACAGAAGCTGCAGGCCAGGAGGCTCCTCAGGACGGCCCCCATCTTCCCTTCTCGCGTTCTCTTCTCTCACCGGCGAGGGG |
| | | TGGCCAGAAGTGGGGGAGGCGAGGAGCCGGGGCGGCCGCCGCAGCGGCAGGGCTGCACGCTCATACTTCCCAGCTTCCCAGC |
| | | GAGAGAAGAAAGAAAGGGGCACGTCAAGGTTCCGCGCGCGCCGCCGCCGCCCTCTCTACCGCGCCGCTCGGCCCCGGGCTCG |
| | | CGCGACGCCAGCGTCTGCTTCCATCCCGCCGCCTCCTCCCCCGGCGCCCCGCTTCCCCCGCGCAGCTCCTCATTCAGCCTCT |
| | | GCCTCGCGCAGCGGCGCAGGGATACGGTCGGCACCGCCTCCTAGGGTCGCCGCGACCGTGTCCCTGCGCGCAACGAGCCCCT |
| | | TCTCCCGGTACTGCCTCCTGTACGGCCAGGGAAGGGAGTCGGCAACCGCACGCACAGCCTCTGCCTGAGACCCTGGGAGAGG |
| | | TTCTGGGCTAGCAGAGGCGAACTGGGAGGGAAAGCCTCCTCCCGGTGGCGCATTCCAGCGGGATTCTTTCCCGGACCGGCCG |
| | | CTTCGGCCCTCCCCGCGGAGGGCGTGAGGCGGCGTTGAACAACATTGGAGCCGGCGTGGTCGGGACTACTTTCTGCGGCTCG |
| | | GCCGGGCAGCCGTCTGCCCCGCTCTTTGTGCGGCCGCCGCCGGCAGGGCCAGGTGGGGCTCCGGTCTCGCGCCCCCAGCCAC |
| | | CTGAGACTGCCCAGCCGCGGTGCACGCGCGGGGAGCCACGGCGGATCCCGTTGCGGGGTGATGAGCTCCGTCTTCGGGGTTG |
| | | GAAATCGGTTTCAGCATCCTTTTTTTGGGAGGGCGGAATTTTTGAACGCTGTTTACACCGGCTTTCAGCGTGGATCTGGTGA |
| | | ACGATATTAAAACGCCAATTCAAAACTGAGATTGTCATTTTTCTCCCGCCTTCCCAGAAATACAAACTGCCCCCAGAATTAA |
| | | GCCCGACCAGCTAAAGATTTCTAAACTGGCAGATAAAATTCCAAGATAAATGCCTCAAAACCTAAGAGCAAAGGGACAATTG |
| | | GACAAGACTATTTAGGATTTTACAAGTTGCATATAATAACGAATGTTTGG |
| 492. | MAGEA3 | GGGTCCTGACCTTGATTCCTGCCACAGCGTGGGCCCTGACCTCTTCTAACCAGCCCTGCCCTGG |
| | | CCACATTAAGCTCTGTCCCCAGAGTTCCCCGTGGCTAAAGCGGCGGGGGTCGGCGGGGTGAGGTAGTGGCGACCCAGCCTGG |
| | | AAGTCTTCCCCTGCGGGGTGGCCCAGGCCCGGCAGCAGAGGCAGCACTGGATTATTTGAGGCCCTCTGTCTGAGGTGAGGCC |
| | | CGCCTCAGTCCTCCCTCAGCGTCTTACCTTGCCTCCTCACCGAGCCTGGGCCGGCTTCCCTCCGCCGACGTCAGGCCGTCGC |
| | | TCGTTGCTCAGGGCGAGAATCTCGCGGTCTTCTGACCTCCAATGCGCAAGTCAGTGGCGTCACATCCTGGCAACGGTACTAC |
| | | CCTGGGTTCCGGGCAGGGGTGGAACTGGATTCTGC |
| 493. | MAP7 | GGGAAGCCTTGCTGCTGGTCTTGGGAAGCCTTGCTGCTGGTCTTGGGGACGTGATCCCCTGTCC |
| | | CTAAGTGTGCCCCTGGAGGTTGTGGGGGAGGGGTATCCTCAACACAGAGGCCTGACCACCAAATCCCGGGCTGCAGAGCCAA |
| | | CAATCCTCCTTTGGGCTCCTGCACAGACTAAAAACAAAAAACTCTGAGAACTCCGACAAGTCAACGAAAGTCTGATCCCGTC |
| | | AAGCCCGCACTCGCAAACCCCCACCGTGCTGCACTGGGCCCCGGCCCTCCCGAGCTCGGTCCCCTCGGACGACGGTCGTCGC |
| | | CACTGCGGGCACCAGATTGGGGGGCGGGCCGCTCACCCCGCGGTCCTGCCCCACACCGACCCCGCTGAGGAGCCCCCGGCAC |
| | | CCGGGGCGACCAGGGAAGGCGGGAGGAGGTGCCCAGAAACTGCTCGTCCTGCCAGTCGCACCTGGCCGACGACTCTCTGGCC |
| | | ACTGGTTTGGTCTGGGGGATACGATTTCCCATTCAACTGAATTAGACCCGAGGCCGCGGCGGGGAGGGCAGCGGCCGGGGTC |
| | | TCTCCGTTTCCTCCCCCGGCTCGCCTCCACCTGTTGCGGGAAAGTCGCGGTGGAGCGCCCGAGGGCGGCCGCAACCCCGGCC |
| | | GGGGCCGAGCCGGGCTGGCCGAGCCGCGGCGGCGAAGGGCGGGGGAAGGCGCTCTCGGAGCAGGGTGCGCGGCGCGCAGGGC |
| | | CGGTTGTTCCGGGCCGCGGCCGCGCGGGCGGGGAGGGGGCTGCCGGCGCCGGGTGATTTCGGTGCCAGCCCGCCGGCCCCGC |
| | | TCGCCGTCCCCTGCCCGACGGGACCCCCACTATCCCCGCTGTGCGGTCACCTGTTTCGCTTCGCACTGCGCCGTCGCCGCCC |
| | | CTGTGGCCGTCGCCGCCAGCTCCTAGCTCCGCCATGGTGCTCCGATGACGCGCCCGGAGAGCCGCTCCGCCCTTTCCTCGGC |
| | | TTGGCTCCCGCCGGCCCCCGCCTCCCAGGCCAGCGGCCCAGACAGGTGAGCGCAGTCCGGGGATGGCGGAGGGCGGGGACAG |
| | | AGAGCGCGGCAGTCGGCTGCGGCCTGGGCCTGGCGCGCCCCCTCGCGGGCAGGTGCGGCGTGGCAGCCGCTCCGACCGCCGC |
| | | TGCCGAGTCTCGGCTGGAAGAGCGGCGGGCGGAACCAGTACCTCAGTCCGGAGCTTCCGGTCGCCGCGGCCGACCAGCTGAG |
| | | GGCTCCCGGAGATATGGAGCGGGGTCAGCGAGGCCTGGTGGCGCCTCCTGGAGTCTGGGACGAGCGGCGGTCCGAGGAGCAG |
| | | CCCAGGTGGGGCGAGGGTACGGAGACGCTATTTCCCTCCGAACTCTGAAGTAGCCAGGTCAGTGTCGGCTCCGCGAGGGCTG |
| | | GAGTTCACGGTGACAGCCGGCAGAGCATCTGATGCCACGTGGAGAGGCGAAGTACATGGGTCGGATTCCACGCTGGGAGCCG |
| | | GCCTGGATCTGCGCTTTAGGAAGCCACTGCGGGCGACCCTATCTTGTCGCCAGGACTTCGCATCTGATGAGCCTCAGGGCAC |
| | | ACAAGTTAGAGAGGCTTGTGCCGGCTGATGTGGCTTTTCTTTTTCTTAGTTTCCGTTTAGTAAACGACCTTGTGACTTCTGG |
| | | CTTGCTCAGGAAGACCAGAAAAAAAGGCAATAGGTAACGAATTTTTCTTTTTTTAAAGAAACAGATTATTGGCCGAGGAAAA |
| | | ACCAATTTCGCTCT |
| 494. | MEIS1 | TTGTAGGTACAGGCAGCTTTTGTCTGAAATCTCAGCTTCCGAAGCGGATTTCTTTTTCTCTGGC |
| | | ACTGGGAGGTTTCGCCGAGCCGGGTTGGGGACGGGAAAGAGGAGCGCGGGGAAGGATGTCTGGGGTGGTGAGGGCAGGGCTT |
| | | CGCTGGAGAAAGAGCTAGTGGGGCGCGAGGTTCTTACAGGCCCGGGAGAGGTCGAGGCTGGAGCCCCTCGGCGCCTCTAAGA |
| | | CAAAGGCAGCGGTGGCTGCAGCCGGAGCCTAGCACTCCGGCAGCGTCGCGCCGCGCCGCGCCGCGCCCTGGGCGCACGGCCG |
| | | CCTCACCCCGAGCGGGTCGGAGAAAGAGCCTCCCTCCCAGCGGCTCCCCGGCCCCGGCTCCGCCCGCGAGGTCTGGGCTGCT |
| | | GCGAGCCCGCGCCGGGTTTCGCTTTCCGACGATCATAAATAGCTTGGTGTTTGTAAACAGGCGCTGGGGGCACATTCCCCGC |
| | | GCTCAGCTCATTGTTCCCTCCCTTCCTCTCTACTTCGCGCAGGACGCCTGGGCTGGGGCTGGGAGCCGCCGGGGCTAGGAGG |
| | | TGGGGGAGTCCAGACCCGAAGTGACAAAATGCTAGCATTTTCTTTCCCCCGGCCGGGCGTCCACCTCTAACAGCAAAGAAAC |
| | | CTCTAAGCTGGGTCTGCAGAAAGCCCGAGCCACCTCAACCCCATGTTCTCAGGACTCCTTAGCAGAGGCTTTCCCAACCTGG |
| | | CTTCTCCCTCCTTTTCCTCCACGATCCCGCTTTGACTTTTCTCCTTGCAGTGTTTAGTTCTGAGAATTCAGTACTTTGCGCA |
| | | TCACCCCCTGCCCCGAAAAACACTGGCAGACCCCAATAATTTCGAGGAAAGTCATGAAGTCTATGCGCGGAGCCCTGTGCAA |
| | | AATAACTCCCGCTGCTGCCTGCCCGGCGTTGATTCCCAATTTATTTCAAGAGAGTCGGCTTTGGGGAGAGAGTCTGCAGGGG |
| | | GAGGGAGAGAAAAGAATACTGAAAATAAAGCTGGCGGCCGCGGGCTACTGCCTGCGTTTGTGTGCGTGTGCCCTGGGTGTGT |
| | | GGTGTTTGTGCCTGGGCGTGCGATTTAATGGAGCGCCTCTCTGCCTCTCCAGTGCGGCCAGAGCTCGCTTCGCGCACCCACC |
| | | CCTGCCGAGGAGCCTACTTGCTGCAGCCCAATGCATTGTGTAAGACGCGACCTGTTATGGCCACCACTACTTCCGGGTTCTA |
| | | GCATTCTGGTCGGAATCCACCTCTCCGCCTGTGCAACACACACTTTACACACGCACGGGGACTGCAAGCGGGCAGCATCGAT |
| | | CGTGGCTCCTTTAAGACAAACTCAGACAGACATTTTTTTTAACCCTCCTTCTCTAATCTCCTTCCAGTGCAGCACTTGCAAA |
| | | GAGGGAGAGAGAGGGAGAGAAAGAAAGAGAGAGAGAGAAGAGAGAAACTGATTAGGAATTAGGACTGATTCAAGGGAAGCGA |
| | | GCGCTAGGGCTTTGTGCATTTGAATATTAACATTTGAGGTGTTCTGACCAGAAGAAGACAGAGCGGATGATCATTCATTCAC |
| | | CACGTTGACAACCTCGCCTGTGATTGACAGCTGGAGTGGCAGAAAGCCATGAGATTTGGTAGTTGGGTCTGAGGGGCGCTCT |
| | | TTTTTTTCCTTTTCTTTCTTTCTTTCTTTTTTTTTTTTTAAACTGATTTTTGGGGGAGAGAAGATCTGCTTTTTTTTGCCCC |
| | | CGCTGCTGTCTTGGAAACGGAGCGCTTTTATGCTCAGTGACTCGGGCGCTTTGCTTCAGGTCCCGTAGACCGAAGATCTGGG |
| | | ACCAGTAGCTCACGTTGCTGGAGACGTTAAGGGATTTTTCGTCGTGCTTTTTTTTTTTTTTTTTTTTTTTTCCGGGGGAGTT |
| | | TGAATATTTGTTTCTTTTCACACTGGCCTTAAAGAGGATATATTAGAAGTTGAAGTAGGAAGGGAGCCAGAGAGGCCGATGG |
| | | CGCAAAGGGTACGTATTAAAAAACAATTGTGGAACTCAAATGTGAGATTAAATTGAAACGTGGCCGAAAGACACTGCTAAAA |
| | | AGTTTCCTTTTTTTCTCTCTCTCTTTTAAAAAATGAGGCTCCTAAAGCCGTGGCCTAAGCGAGAGGATTTATTCAATGCATT |
| | | TGTAGACTTATGTATGTTACTTGGAGTTGTTAATTAAAGGAATCTATTTATCTATAGATTCCTAACCAGCAGCTATATAAAT |
| | | AAAAATGCAAGCTCAAACAATCATCTGGGTTTTTTTTTAAAAAAGCTAGATACCTAAAGCTGTAATTTGAACATTCAGAGAT |
| | | CAGAAGTTTAAAGTTATG |
| 495. | MGC14376 | CCCTTCCCTGCCAGGGCGCTGAACTGGCCCTGACCCAGTGAACGCCGGCAGAGGCAAAGCACCT |
| | | CCTCGCCGCCCGCTCGGGGGGTCCAGACCCTCCCCAGGCTAGATGGCCGGCCCCTGTATCCTCAGACCGGGGGCCGCCGGGT |
| | | GAGCCGCCCCCGGCCCTCCGGCCCCTCGGCTCTCCCCCCGCCCCCCGCCACTCCGCCAGCCCTTCCGCCACGACTCCCGTCC |
| | | CAGACTCTTTCCTCGTAGCTCCTGACACACTAACCCGGCCAGTCCAGGCCCCTCTCCCCGAGGCGCCCCTGCCCCCGCCGCC |
| | | CCTTCTCCTCCCGGAAGGGCTGGGTGTGGCCGCCCGGACCCCACTTTCACCCTCCATCCCGTTACCGCGGCTCCCCGCTGCG |
| | | TGACCGGGTCGCCAGCTCACACCCACCTGGTGCTGGGTGTGGGGGTTGCTGCACGAGGGGGAGCAAATCACTGCGTCCGCTC |
| | | CCCACGGCCGGCGGCGCTGTCTCTTCTCGGCACCCAGCACACACCGGCTCCAGGCTCCGATCAGCTGGCGGCGTCGTGACGC |
| | | ACGAGCTGCGAATGCACGGCCCGGGGCCAATCACCGCCCACTGCTGGGCAACGTGGCCCCGCCCACCCGGGGGCCGACGGGG |
| | | CCTCGCCCTGGTCCCGCCTCTCCGGGCGCCCTGGCTCTGATTGGCAAGGAGGCCACCCTTTTGCCGGGCGGAATTGCACGAA |
| | | GCCACGCCCACCCACTGAGGAGCCGGCCAATAGACGGACAGGTGTGGCCCAGCCCTCCGCGTCAGCGACCCGCTTAGAAAAG |
| | | CGCTACGTCAAACGAGAGTCCTAAAGAGCAGGCGAAAGCCACGGCGTCTGCGTTTGCAATGCATGCTGGTCCGTGTAGTTCT |
| | | CAGCCTGACACCGTCGTTCCCAGAACCCAGCGCGCTCTGTGAGTTGGCATTTTTAAACTGAGCTCGGAATCCAGCCCGGGGA |
| | | AGTCCTTAAAGGGCGACAGCCCCTCCTTGTTGGAGTCGTGAGTGCTTCTTTTAGACATTCCCCAAACTGACTCGCCGGCCTC |
| | | TGCGGGACGCTATAGCTCTTTAAGGAAAGGTGGAGCGGACCAAGGAGCAGGAGTGGGCAGAGCGTCAGCCCCCAGGGCAGCA |
| | | GAGACTGCCGTTGGAGAGCTCTCCAGGGTTTTTGGAGGAGGGAGGGTGCTCGCCTCGCCCAGCCCACTCCTCCTGGGGGCTC |
| | | TTCCTAACCACAGGAGGCTTTTTTTCTTGGATGTCTCTTCCCAGT |
| 496. | MGC16121 | TCCTCTCCCCTTACTCCTTTTCCAGTCCTCCCCACCCCCCAGCATTTCGTGGGTGGGGACTGAA |
| | | GATAGGTTTTCTTCGTTGTCCTGTGGGTGGTATTCTGATTGGGAAGGGATTTTTTTCAATTGTTTACATTCTACCTGAGCAG |
| | | GGAAAGGGGGTGCCAAAATGAAGCATCATTCACGTCCAGTCACATATGCAGAGACGGGGCAAACGTTTGATGCAATCTTGGG |
| | | TCTCGCTGCTCCCGGGGCCGACACCGCAGGCGATGGCCTAAGACTTACCTGCTGGGTAGGCCCGAACGGCAGTCCCAGACTT |
| | | ACCCTGGCAGCGGAAACAATACCCCAGAGCACCGATCGCTCACTGCAGAACTGTTCCCGCTGCTAGGGCACGGCTGAGCGCG |
| | | GGCACCGCGTCTCTCCTCGACTCGCACCGCCTATGCGCCCCAGCCTAGCCAGGAATACTGCCCTCCCCGGACTACAGCCCTG |
| | | CTGCGTGGACAAGGGCGAGGACCCGCCGGAAACAGAGACGCGAGCCCCCGCCCACCTACCTGCTCCTGAGCCGGCCTCGCGG |
| | | GGAGCGGGCACCTGGTGGCAGGAACACGCGTCCGGCAGACCCCACCTTCTACCTTCCCCACGAGGGGGTATAGCAGCGCCTC |
| | | ACGTTTTGAACCATTTAGAACACTTCAAAACATGAATTGCTGCTGTATCCCCTCGGAGTCAATGAAGGGGGATCGATCTGGG |
| | | GGTACCTGCGGGTGAATAAAGACTGGATGACTGAAGGAAGCCCCGCCCCACCCCCTGGCAAGGAATAGGGCAAAACTTATGA |
| | | CTAGGCCACTTCACTCCAAGTTAAGGTACTTCTTATAAC |
| 497. | MGMT | CACCACAGGCCCTGGAGGCTGCCCCCACGGCCCCCTGACAGGGTCTCTGCTGGTCTGGGGGTCC |
| | | CTGACTAGGGGAGCGGCACCAGGAGGGGAGAGACTCGCGCTCCGGGCTCAGCGTAGCCGCCCCGAGCAGGACCGGGATTCTC |
| | | ACTAAGCGGGCGCCGTCCTACGACCCCCGCGCGCTTTCAGGACCACTCGGGCACGTGGCAGGTCGCTTGCACGCCCGCGGAC |
| | | TATCCCTGTGACAGGAAAAGGTACGGGCCATTTGGCAAACTAAGGCACAGAGCCTCAGGCGGAAGCTGGGAAGGCGCCGCCC |
| | | GGCTTGTACCGGCCGAAGGGCCATCCGGGTCAGGCGCACAGGGCAGCGGCGCTGCCGGAGGACCAGGGCCGGCGTGCCGGCG |
| | | TCCAGCGAGGATGCGCAGACTGCCTCAGGCCCGGCGCCGCCGCACAGGGCATGCGCCGACCCGGTCGGGCGGGAACACCCCG |
| | | CCCCTCCCGGGCTCCGCCCCAGCTCCGCCCCCGCGCGCCCCGGCCCCGCCCCCGCGCGCTCTCTTGCTTTTCTCAGGTCCTC |
| | | GGCTCCGCCCCGCTCTAGACCCCGCCCCACGCCGCCATCCCCGTGCCCCTCGGCCCCGCCCCCGCGCCCCGGATATGCTGGG |
| | | ACAGCCCGCGCCCCTAGAACGCTTTGCGTCCCGACGCCCGCAGGTCCTCGCGGTGCGCACCGTTTGCGACTTGGTGAGTGTC |
| | | TGGGTCGCCTCGCTCCCGGAAGAGTGCGGAGCTCTCCCTCGGGACGGTGGCAGCCTCGAGTGGTCCTGCAGGCGCCCTCACT |
| | | TCGCCGTCGGGTGTGGGGCCGCCCTGACCCCCACCCATCCCGGGCGAGCTCCAGGTGCGCCCCAAGTGCCTCCCAGGTGTTG |
| | | CCCAGCCTTTCCCCGGGCCTGGGGTTCCTGGACTAGGCTGCGCTGCAGTGACTGTGGACTGGCGTGTGGCGGGGGTC |
| 498. | MGP | GGGCCTGTCTTCAGAAGAGAAAATGGGGGAAGAGCTTTGAACCCAGAGGTACAATCTCTGGATT |
| | | GGAGAGTTTCAAAGGTGCCAGGGAAAAGCAGTACAAAGAGCCTTCTTTTTGACAATAACTCTAAAACACACTTTGCTCTGTG |
| | | AACGCCCGTCCTGGGCAGGGCCTATCAGCAGGGCCAGATGAGCCTATAGACACCCAGGGTGGCGCGTTCCCAGCCGCCCTGC |
| | | GCATGCCAATCTTATCAGTTTTGATGGAAGCCTGGGGCTCCAGATAATGCTACTTGGCCCCAAACGCTGGACAGAGCATGCT |
| | | TTTTCCTGTTTGGCTGGGAAAGGGAAGGCTGAACTGCTGAGTCTGAC |
| 499. | MIG2 | TCGAATCATTACATATGAAAATTCAGAGAATAGCAAAACAAAATAAGACGCAAAGGGGAACAGG |
| | | TCTATCTGCTTAAATACTGAAAGATTTTACATCTCCAAAAAGCATTTGCGAGATGCCAAGATTTCTCAAATGGGCCCCTCCC |
| | | CCGTCGTGAGCCTCTTTCCTCCCCCTACCAATTCTGGGAAGTCATTAGTTGGACGCGGCTGGGATGCTACTCACCGAGTTTC |
| | | TCCACCAGCTTAAGCATCACGCCTCCAATGTGCACCTCGCCGGTCACTCTCAGGGTGACATCGCGGTTCAGGTCCGTCACAT |
| | | GGACACTCAGTTCCCACGTCCCGTCCGCGTAGCAGCCATCTGGCATCCTTATCCCGTCCAGAGCCATGGCTCCTTCCTGCGA |
| | | GCGCGGAGGAAATGGCTCTCGTAAGCGTCACTCCCCCAAAGAAAGGCGAATCCCACCGAATTCGCAGCGCCGGCCACGGGCT |
| | | GGGAGGTGGTGGAGGACCCGGGGCTTTCGGCAGAAACTCGGGAGGGCGGCGGCGGCCGGGGCTGCGGGAGGACCCTACGCCC |
| | | CGCTCGCCCCGCAGCGTTCCTCGGTCCCGGCGCCGCCCGCCCCACACCGTCCCCGCCTAGCGGACCGCGGAGGGCTTCACCT |
| | | GCCGGCCGGCCCCGAGACACAGCGCGGGCTGACTCCCCGTTCCCCTCCCCGCCGCGCCCCCTCGGGTCCCGGCGGGGTCCCG |
| | | CTCCCTCACCGCGCGCTCCTAGCGCTCCGGGCCCGGGACTCGCGCGGCAACAGGCGAGGGGCTGGAGGCTCGCGGGGCGGCG |
| | | GTGTCCGCGTCCGGTTCCTCGGCTGGCGAAGCGCTGCCTGTGGCCGGAGCGGCTAATGGAGTCCCGTCGTCGCGGCCCCTCG |
| | | CCTGGCTCCCCGCGCTCCACCCTCTCCCCGCCCCCTCTGTCCGAGCCTGGCTGGGCTGGGCGCGCTGGCTCCCTCCTTCGCC |
| | | GCCCGCAGAGCTGCCCTGAGCGGGTCCGGCCGGCCTCGCCTCTGCCCCCGCCCTCCCGGCGCCGCGCTCGGGGGAGGGGGCT |
| | | GCGGGTCCCGGGCGGGGCGGGGCGGCGTGGGGCGGGGCGGCGCGGGGGTGGCGGGGGGCGCGGCGGGGCGGCAATCTCGGCC |
| | | CCGCGGAGCGCTGCCCTTTTATGGAAATGAAGGACCCGGCTCAGGAATTGAATCCAACATCCGGGCTCTAGGCGGCGGGCCC |
| | | TGAGGAGGGGGAGCGGGGGAGCGCGGGCGGCCTTTCCCGGGGCGCTGGTGGCCGGCGGGGCGTCTAGAGCTCAGGCTGGACT |
| | | GGGGCCCCCGCGGCGTGTGTCCGCGCTGGCAGCAGGGAGAGGGGGCTTGTCCGTGAGAAACGGCCCCAGGAAATTCCCAGGG |
| | | CAAGGTCGTTTTCGGAGAACAAAGTCCTCTCGCCCCCTCCCCACGCCTGCAGCGAGACAAAAAGCCTCAGGGAGGTGAGAGG |
| | | TGCCGCACCCCGGGAGCTGGAGAGAGGAGTCTGGGACGGAGAAACTTTAAAAAGTAAATGCCCAAACCAACTCCTTGAATGT |
| | | CCTACAAAAGAATAAGAAAACCCCGAGGGAGGTGAACCACAAAAATTGCTTTGCAAGCAATGTCCGGCACCATCAGCAACTC |
| | | CTACTGGA |
| 500. | MSLN | AGGAGGCTGAGGTAGGAGAATTGCCTGGACCCGGGAGGTCGAGGCTGTAGTGAGCCGTTATTGC |
| | | ACCACTGCACTCCGGCCTGGGCAACAGAGCAAGACCCCATCTCAAATAACAGTACAAAATAAGTATGGAGATGCTGGCTCCT |
| | | GTGGGAAGCTGGGTGGACGTGGGGGGCACACCCTCCAGTCTGCAGAAACTTCCCGCCGCTCGGGCTGATGTGCTCGAGGGCT |
| | | CCCGAGGCCCTGCCTCCCGGGTGGCAGAGGGAACATGGGAAAGGCTGCCGGGCCCCGGGCTCGCCGGTGGGTGGCCCCGTGC |
| | | AGCCTGTTATTCATAAAACTTGGAGAGAAAGTGCCTTTTTGTAGGAAAGTGTGGGGTGCTGGCGGCCATTTCCACCTGTGGT |
| | | TGGACCCAGACTGCCTCCCCGCGTGTGGCCCATGCCCCACCCCACACGCACCCCCGCCCTCTGTCAAGCAGGACTCGGGCTC |
| | | TCCGTTGGCCGAGCAGACGTTTTATTTGTGGAACGGAATTGCTGTCGGGATGGGCGTGGGGTCGTTAGCAGCATAATCAACA |
| | | CCCACATCCACCCTGGGGGCAGCCACGTCTTGTCAACAGGTGGAGGCCGTGACATGCTGCAATTAGCAGCTAATGAGGAGGC |
| | | CTGTGTGACCCACGCCTCCGTCACAGGTGCGCAGTGAGCTGTGGAAACGCCGACAATTTAAAATAGTCGAACAGGGCCGGGC |
| | | GTGGTGTCTCAGGCCTGT |
| 501. | MYOD | TCTCCAAATCTCTCACGACCTGATTTCTACAGCCGCTCTACCCATGGGTCCCCCACAAATCAGG |
| | | GGACAGAGGAGTATTGAAAGTCAGCTCAGAGGTGAGCGCGCGCAGCCAGCGTTTCCCGCGGATACAGCAGTCGGGTGTTGGA |
| | | GAGGTTTGGAAAGGGCGTGCCGGAGAGCCAAGTGCAGCCGCCTAGGGCTGCCGGTCGCTCCCTCCCTCCCTGCCCGGTAGGG |
| | | GACCTAGCGCGCACGCCAGTGTGGAGGGGCGGGCTGGCTGGCCAGTCTGCGGGCCCCTGCGGCCACCCCGGGGACCCCCCCA |
| | | AGCCCCGCCCCGCAGTGTTCCTATTGGCCTCGGACTCCCCCTCCCCCAGCTGCCCGCCTGGGCTCCGGGGCGTTTAGGCTAC |
| | | TACGGATAAATAGCCCAGGGCGCCTGGCGAGAAGCTAGGGGTGAGGAAGCCCTGGGGCGCTGCCGCCGCTTTCCTTAACCAC |
| | | AAATCAGGCCGGACAGGAGAGGGAGGGGTGGGGGACAGTGGGTGGGCATTCAGACTGCCAGCACTTTGCTATCTACAGCCGG |
| | | GGCTCCCGAGCGGCAGAAAGTTCCGGCCACTCTCTGCCGCTTGGGTTGGGCGAAGCCAGGACCGTGCCGCGCCACCGCCAGG |
| | | ATATGGAGCTACTGTCGCCACCGCTCCGCGACGTAGACCTGACGGCCCCCGACGGCTCTCTCTGCTCCTTTGCCACAACGGA |
| | | CGACTTCTATGACGACCCGTGTTTCGACTCCCCGGACCTGCGCTTCTTCGAAGACCTGGACCCGCGCCTGATGCACGTGGGC |
| | | GCGCTCCTGAAACCCGAAGAGCACTCGCACTTCCCCGCGGCGGTGCACCCGGCCCCGGGCGCACGTGAGGACGAGCATGTGC |
| | | GCGCGCCCAGCGGGCACCACCAGGCGGGCCGCTGCCTACTGTGGGCCTGCAAGGCGTGCAAGCGCAAGACCACCAACGCCGA |
| | | CCGCCGCAAGGCCGCCACCATGCGCGAGCGGCGCCGCCTGAGCAAAGTAAATGAGGCCTTTGAGACACTCAAGCGCTGCACG |
| | | TCGAGCAATCCAAACCAGCGGTTGCCCAAGGTGGAGATCCTGCGCAACGCCATCCGCTATATCGAGGGCCTGCAGGCTCTGC |
| | | TGCGCGACCAGGACGCCGCGCCCCCTGGCGCCGCAGCCGCCTTCTATGCGCCGGGCCCGCTGCCCCCGGGCCGCGGCGGCGA |
| | | GCACTACAGCGGCGACTCCGACGCGTCCAGCCCGCGCTCCAACTGCTCCGACGGCATGGTAAGGCCGGGACCCCAGGAAGTG |
| | | AGGAAGTTAGGGCGGCGCTCGGGATATCAGGGACGCGTTTCCGAGGGCGGGGAGCTGGCCTTGCGGGAGGTTTGGGCCAGGA |
| | | TCCTTCCCGAGAGAGAGGACCCCCTTGTCCTGGGCAGCTGTCACTGGGGTAGCCTGTTTTGGAAGTGTGCGGGCAAGCGTTC |
| | | GAGCTGCCCCATTGGGGGCGCTATTAGAACACTGCAGCGCGAACGTGAAGATCTTTTTCTCTACTTATCCCTACTTCCAAAA |
| | | TGTAAATTTGCGCCCCTTGGTGACTGTCCGCCCTTGGTTTGGCCCTGCATGTTGCAGACCTCATCTCCTACCCACCCGTAAT |
| | | TACCCCCCCAACCAGGACAGGTCTGGGCCCGGAACTAGAGCCTTAGGCTAGAGTTAGGGAGGGGGCGGCTACAGGAATTGGT |
| | | GTTCGGGCCTCGAGCCGTCCCGCGGGCCTGACTCAGTCGCCCTTGCTGTTTGCAGATGGACTACAGCGGCCCCCCGAGCGGC |
| | | GCCCGGCGGCGGAACTGCTACGAAGGCGCCTACTACAACGAGGCGCCCAGCGGTGGGTATTCCGGGCCTCTCCCTGCTCGCT |
| | | CCTCCTCCTTCATGGAGCTGTCCTGGCCTCTATCTAGGACGCTCCCACCCCCACTCACACACGCCTATGTCCTGGGAAGTGG |
| | | TGCAGGAGATGAAATACTAAGCAAGTAGCTCCCTGTCTTTTGGATTGTCCCGGACTCTAACTAAAGTCCTCAGTTTCCAATC |
| | | TGTCTCAAAGTACTGGGCCCGGGGGTGGGAGGCTTGTCGCGGCCCCACCCCTGCTTACTAACCGAGCCCTCCCCGCGCAGAA |
| | | CCCAGGCCCGGGAAGAGTGCGGCGGTGTCGAGCCTAGACTGCCTGTCCAGCATCGTGGAGCGCATCTCCACCGAGAGCCCTG |
| | | CGGCGCCCGCCCTCCTGCTGGCGGACGTGCCTTCTGAGTCGCCTCCGCGCAGGCAAGAGGCTGCCGCCCCCAGCGAGGGAGA |
| | | GAGCAGCGGCGACCCCACCCAGTCACCGGACGCCGCCCCGCAGTGCCCTGCGGGTGCGAACCCCAACCCGATATACCAGGTG |
| | | CTCTGAGGGGATGGTGGCCGCCCACCCGCCCGAGGGATGGTGCCCCTAGGGTCCCTCGCGCCCAAAAGATTGAACTTAAATG |
| | | CCCCCCTCCCAACAGCGCTTTAAAAGCGACCTCTCTTGAGGTAGGAGAGGCGGGAGAACTGAAGTTTCCGCCCCCGCCCCAC |
| | | AGGGCAAGGACACAGCGCGGTTTTTTCCACGCAGCACCCTTCTCGGAGACCCATTGCGATGGCCGCTCCGTGTTCCTCGGTG |
| | | GGCCAGAGCTGAACCTTGAGGGGCTAGGTTCAGCTTTCTCGCGCCCTCCCCCATGGGGGTGAGACCCTCGCAGACCTAAGCC |
| | | CTGCCCCGGGATGCACCGGTTATTTGGGGGGGCGTGAGACCCAGTGCACTCCGGTCCCAAATGTAGCAGGTGTAACCGTAAC |
| | | CCACCCCCAACCCGTTTCCCGGTTCAGGACCACTTTTTGTAATACTTTTGTAATCTATTCCTGTAAATAAGAGTTGCTTTGC |
| | | CAGAGCAGGAGCCCCTGGGGCTGTATTTATCTCTGAGGCATGGTGTGTGGTGCTACAGGGAATTTGTACGTTTATACCGCAG |
| | | GCGGGCGAGCCGCGGGCGCTCGCTCAGGTGATCAAAATAAAGGCGCTAATTTATACCGCCGTGGCTCCGGCTTTCCCTGGAC |
| | | ATGGGTGTGGGATCCGGAGGAAAATCCGCAAACTGGGCCAGCTGTCCCTCAGCGACGCCTGTAGGCGGCAGGCGGATTGCAA |
| | | GGAGGAAGCCTGCTGCCTGGGGAAGGAAGGAGGGGTGCAAATTTCTCCAGTACGTGAGGAAGTTCCTCTGACCTTGACTACA |
| | | TTACTACACA |
| 502. | N33 | CCTCAGATTGAGGTCCCAGGGCCAAAGGACCACTCCTCTCCTCAGCGCTGGTCCGGGAAAGGCA |
| | | AGCTCCGGGCGGGAGCGCACGCCGCGCCCCCGAAGCCTGGCTCCCTCGCCACGCCCACTTCCTGCCCCCATCCCGCGCCTTT |
| | | CCAGGTCTTCTCCCGGTGAACCGGATGCTCTGTCAGTCTCCTCCTCTGCGTCCTCGGCCGCGGCCCGGGTCCCTCGCAAAGC |
| | | CGCTGCCATCCCGGAGGGCCCAGCCAGCGGGCTCCCGGAGGCTGGCCGGGCAGGCGTGGTGCGCGGTAGGAGCTGGGCGCGC |
| | | ACGGCTACCGCGCGTGGAGGAGACACTGCCCTGCCGCGATGGGGGCCCGGGGCGCTCCTTCACGCCGTAGGCAAGCGGGGCG |
| | | GCGGCTGCGGTACCTGCCCACCGGGAGCTTTCCCTTCCTTCTCCTGCTGCTGCTGCTCTGCATCCAGCTCGGGGGAGGACAG |
| | | AAGAAAAAGGAGGTAGAATGGATCCCCTTGGCCTTC |
| 503. | NBL1 | GAACCCCCGAGGTGAGGCTGGAAGCTGGGGCCAGGAGCTCCACTCTTCCCAGGAGTCAGAGAAG |
| | | TGAGTTCATTTGCTGGCTTTTTAGTTCCTAATAAGTCCCTGGGGCTTTTCGGGATGCTTAAGCCAAGGGCGTGGGGCCGAGA |
| | | GCGACACTAGGATAACACTAGGCAAGGCTGAGCAACCCAGGCTACCAACAGCACGGACTTGAACCGGGTCGCATGCCGGAGC |
| | | GGACGGGATGAGGTGCACCGCAGAACTGGGGACTCCTGGTGCCGGGACGCGGCGGGCTGGGGCGCCCCACTCTCGGGGCAAG |
| | | TCTGCAATGCCCGGTGCCCACAAAAAGGACACATCCATCTTTGTCACCCCGAGGCCTGGCACGGCCTGACGGCCCAGGCTCG |
| | | GTAAAAGTCGGGCGGAGAGAGTGGAAGCGCAGAGAAACCCGGGGAGGGCGCTTAAGAACGGGCCAGGGCGCCCGGCTGGGCT |
| | | GCCCTGCCTCTCGGGCGCCGGCCAGGCGTCCCGGCCGCCCCACCACGCCGTCGGAGCGCGCCGAAGCTCAGCCCGGGGCGGG |
| | | CGGGCCAGGAGAGGGCCCGGGGCCCCTGCCGCCGCGGCGCCCCCTCCTGCGCACCCGGAGGGAGAGGCCGCGCGCGCCCGCC |
| | | CGCTCTTTCTGCGCGCGGCCCTCCCCGCTGCCCCCGCCCTCGCGCCCTGTTTGGAGGGAGCCACCCTGACGTCGGAGCCGCT |
| | | CCCCCGCGCCGCGCGCCCGCCCGGGGCCGCAGACAGCGCGCAGCGCAGCCCAGCCGAGCGTCGCGGGGCCGCCCCCCGCCCT |
| | | GCCGGCCGCCTCGCCGAGCCTCCTGGGGCGCCCGGGCCCGCGACCCCCGCACCCAGCTCCGCAGGACCGGCGGGCGCGCGCG |
| | | GTAAGTCCCGCCCGGGTCGGCACGCGGGCGCCCGGCTTCCAGAGGCTTCGGCCGCGGGGGCAGTGCCGCGCCCCCAGCCCGG |
| | | AGCTGCGTCCCCCGGCGCGTCCGGCGGCCGCGGGCACCGGGTGGAGGCGCCGCCGCCGAGCTCAGGAGCCCTGGGGGACCTG |
| | | GCGGGCGCCTCCGGACGCCGGCGCTGGGGACGTGGGCGCGGCACGGGGTGGCCGGGGGGCACCGCCGCGTCCGGAGCCCGTC |
| | | CCCAGACTCGCCCCAGGGTTCCGTTTCTGGATCGGAGTTTCCGCGGCCGGGGGAGAGAGGGAGCGAGGGAGGGAGATGCGTG |
| | | GCCGGGCGGGCCGGGCTCGCATGTCCCCCGGCCGTGCCCGGGCCTCGCCGCGCCGCGCCTCTCGGCTCTGGACGTTTCCGCC |
| | | TCCCGCGGCCCCCTCTGCTTCGGTCCCCACGTCCGTGTGTCTGTCTCTCCGTCGCTCCGTTTCCGCCGCTCACTTTCCCCTG |
| | | GTCTGCCCGTCTCTTTCCGTTCCCCGCCTGCCTCCGTGTTCGGCTGCCCGCGTTTGTGTCTCTGCCTCTGGCTTTTCTCTCC |
| | | CTCTCTCTGTCTCTGCGCTGCCCTCTGTGGGGGTCTCGCCCTGTGTCTGCGCCTCTCCCTGATCCCCCGCCCTCCGATCCAG |
| | | ACAATCCCCCAGTGTCGGAGGCTCGCCTTCCCCCAGCGGGGTTCACGAATAGTTTCCTAGACCGGGTGCGGCAAACCCTCCC |
| | | CCTGCCTCCCGCCGGCCCCGAGGCCTGGCCCGGGTCATCTCCCTGGAATTGAGTCTGGGGCAGGGAGAGCCAGCCTTTCCGG |
| | | GGGCTCAGTTTTCTTAAGAATTCCACCCCCGCGAGGCCGGCTGGACGACAGGCCCTGCTGCGGGCCACCTGCCCGGCCCGCT |
| | | CCCAGGGCGGGGCGATTCTGATCGTTTACAAGCGGCGGCAAAGGGGGCAGCCGCCTGCCTTGGCGTGGCCGGAGGTTGGCGG |
| | | GCATCAAGGGAGAAAGCCACACTTCCAGGCTTTCGGAGGCCCTCGCTCCCCAAGCTGGACGCCACCAGCCTGTAACTAGGGG |
| | | CACCCCAGGAAAACAGAGGTCAGGGCTGGAGGCCAAAGACCGGGCGCTGGCGGGGAAGCAATTTGTCAACAAGGAGCCCCTC |
| | | ACCCTTTGTTACTGCGGAATGGGGCCTCCCTAGAC |
| 504. | NFIB | CTACGCCCAAATATCGTCAAATGAACAGGTTTCCACAAAAGGAACACGTTCTAAAAACTCCTTA |
| | | ACAATCACCAAGACCAACTAACTCGTGTCAACGTAAATACTGCCTTTGTGGTGCGGTCACTAGAAACCCCAAAGTGCAGCAG |
| | | GTCACTATGGACCAGGGTTTCAGATCCCCAAATAAAGCAAAGGAATGGGTCCCCGGACCACAACCTCCGACTCGATTCGTCG |
| | | CCCGCCCACCGCCTGCAGCGCAGTCCAGCGGCGCCCACACAGACTCGTGCTACAGTCCCCTCGCCCAAGTTCACTGGCTGTT |
| | | TTTAAAAGCTTAGTCCCCCGTAAAGTCCTCCAAAGCCCGAGTCCACCTCAACGCGCGAGCTGCTGAGAGGGGCTACGGGCAC |
| | | CCCGGGCGGGGATGCCGCACCACAACGGGCACTTGAGGGGCCGCACGGGGCCTCGCACTTACAGGTCCCGGCCCTGCCCACC |
| | | CCCCGGCGGCCTTGCCCGGCCCAGCGCCCGCGCTCCGTGCCCAGGGCGCGGGGCTGGGCGCTCGCAGTGGCCGTGGCGAGGG |
| | | GCCGCTCCCGGCTCCCACGCCGCCCCGCGACGCCCGCTGCAACTCCGGGCCACTTCTCCAAGGGACGGGGATGTGCGGAGGT |
| | | TAACTCAAGCCGCTAATTGTCCGCAACAAAACAAAACAAAGGCATTTCGGGCCAGAGAGAAAGCTCGAGAAAGCGACCGAGA |
| | | CATGTACCTGAGTGAGACAGATGGGAGAATACATCATGACTTCGCCTTAAAACGCACTTTCCGGGAGATGCCCAAGAAAATC |
| | | TTCGAGAAGCAAGAATTTCATCTATTCATTTTACAGTCATCTGAGCCCCGCGATGCGATCAATCAGGACGGGGCTCTGCGCT |
| | | GGATCACCGCAACTTCACAACAAACCCAGTCCTCCTTAAATAGCCAAAGATTCAAGTTCACTCGGCGTGCTAGATTTCCAGG |
| | | GGTGAAATCCAATCTACACTTTTAACCCTCTTGCAGTCCGAGCGCGCTGGCCGTGCTTGCCGAGGCCGCCGCCGCCGCCGGT |
| | | GTTGGCTGCTTTTCGCCTGGGTTTGGGGATTTGTTTTCTATTTTGCAGTTGTTGTTGTTGTTGGGGTGTAGGGGGTGCGCGA |
| | | AGGTTCGGTGTGGGTTGGATTGGGGTGGTGGTTGGTGGTAAAATGCTTTTTCAAAAAAGGCGGGGAGGGGGGCGCGGGAGGG |
| | | CGCAGGAGGGCGAGCGGGCGGGCGGGAGGGAGAGCGGGGAGAATGTGTCACCGCGCTGGGAAAGTTCAAGGTTACAGCCCCA |
| | | AGCACTGCGGCAGGATCCCGGAGTGGTGATCGCAGGCGAAACTTTGCCGCGAGCCGACCATGTGTGTGCGCGAGGGGCAGCG |
| | | TGAGCGAGTGCGCGCGGGTGGCGGGGCGCGCGCGGGAGAGGGCCGGGGTGGGGGCGGGGTGGGATGGGGGAGAAGGGAGAGG |
| | | CCGGGGTGAGGGAGGGGGCGCGAGCGCTGATCTCTGGGGCGGAAGAGGCTCGCGGCGCGTGGTCCCCGGCAGCAGCAGCCGC |
| | | CGCCGCCCGCGCCGGGTCTTCGGCGCCCGGCCGCCCGCCCGCCCGCCTCGCCCCGCGTCTGACCCGGCCGAGCGTGGAGGCT |
| | | GCTGTTGCCGCCGCTGCCCAGAGCCCAGGGGTATCAAAACGATCTCCTGAAAAATTCCAAACGATAAATCCCGCTCTCCCGC |
| | | TCGGCTCCAAACTCCTCTCTTTTCTGCTCTAGGCTCTTTTTTTTCCCTCCCTCTCTTGCTTTCTTTCGTTGCAAAACTTGGA |
| | | AGTTGAAAAATTCACCGGTTCCACCCTCTGGACCCCGCTCGAGCTCTCTCCAAATCAGACTTAAGGATTGTTTTATTATTTA |
| | | ATTACACAATCATCGCTTTACTCCTCCTCCTGCAAACGCGCATTCCTTTTGCACCAGCCTCTCCACACCCCCCGCCCCCCCA |
| | | CCTTCCTCCTCCTCCTCCTTCTCCTTCTCTCCCCCCACCTCTCCCTCATTTGTTAAAGGTATCCTACCGGTGCTACAGAACC |
| | | ATGACTTTTTTTTTTTTTCCAAAATAGCTCTTCTTTCCGCTGCCCTCCCCACACCAGCACACACATACACACCCCCGAAAAC |
| | | CCGCCCTGGAAGAGGTATTTCGGTGCTCGCTGCGGACTCTTTTAAGCCGCGGGAACATCCGAGTGGGAGAAGCACCGAAACC |
| | | GTCTGAGCCCGAGAAAGGAACGAGTGGAAAATTCCCTCACACCCAGGCCGCCCCCAGGCCGGGGCTGGGGCGCGGACACTCG |
| | | CACCCGGGGCGGGCTCAGTCCGCGGAGCCTGCGGCTGCCGGGAAAAGGGGGAGGCCGAGCAGGCGGGGACCTGAGCGAACTT |
| | | GGGCTCAAGTAGTTGGGGCGCGCCGGCGTGGACCAGGGGGGTGCGGCGCCCAGCCCCCGACGGCCCCGCCCGGAGCCCGGGT |
| | | TCGCGGATAGGGAGGGGGTGCGGGTTCGTCCCGGGTCTGCCCACCAGGGGCCCGCACCCGAGGCAGTCGCGGCGCGCGGCGC |
| | | TTCGCCGCGGTTTGCCGCCCTCCCCGGGGGTGCCCCGTGCACGTGGCCTCGCTCTGAGCGGGAGGACCGGGCCGAGCCGCCG |
| | | CCGCCGCCTCTTGCTCCCTCCACCTCCTCCCCGCGCCCGGCTTCGCTCTCCTCCTCCTCCTCCTGCAGCCCCGCTGGCTGGC |
| | | TCCCAATCCCCACCCCCCGGGGCCCGGGGCGCCGGCGGAAAGCGTCTCCCTACCCGCCCGGGTGCAGGCGGGGCGGGGCCGC |
| | | GGCGCGCCCGGGGCTGCGGGGCGGGCCG |
| 505. | NFKB1 | AAAAACCAGTAGAGGGTTATACTTTACTGGGCACAAGTCGTTTATGATAACGAAATTGTAGTTT |
| | | AATCTGTGAAGAGATGTGAATGTAACTGAGACACGCTTAAATGGAATATACAGATGAGCTTTATTTTTATATCTGGCATGCT |
| | | TGGATCCATGCCGACCCTCCAGCTGCTCGGGCCTGCCCTTAGGGGCTATGGACCGCATGACTCTATCAGCGGCACTGCCACC |
| | | GCCGCCGCCTCCGTGCTGCCTGCGTTCCCCGACCATTGATTGGGCCCGGCAGGCGCTTCCTGGGGGCTTCCCTACCGGCTCC |
| | | AGCCCTTGGGATTCGGGAGCGCCCTGCTAGGAAGCCAGAGCCCCGCAGGGGCCGCGGCGTCCAGGCCGCCTAACGCGCGCCC |
| | | CTCGCCCGGCGCCCCGAAGCGGCCCCGAGGGGCGGGAGCCGAGGCGAGCGGCAAGGCCGGGCCGGGGGCGCACAGCGCCCCT |
| | | AGAAGTGCGGGCTTCCCCCACCCCCGGCAGCGACCCTACCTCCCGCCCCCGCTGCGTGCGCGCGTGTGTCCGTCTGTCTGTA |
| | | TGCTCTCTCGACGTCAGTGGGAATTTCCAGCCAGGAAGTGAGAGAGTGAGCGAGACAGAAAGAGAGAGAAGTGCACCAGCGA |
| | | GCCGGGGCAGGAAGAGGAGGTTTCGCCACCGGAGCGGCCCGGCGACGCGCTGACAGCTTCCCCTGCCCTTCCCGTCGGTCGG |
| | | GCCGCCAGCCGCCGCAGCCCTCGGCCTGCACGCAGCCACCGGCCCCGCTCCCGGAGCCCAGCGCCGCCGAGGCCGCAGCCGC |
| | | CCGGCCAGTAAGGCGGCGCCGCCGCCCGGCCACCGCGCGCCCTGCGCTTCCCTCCGCCCGCGCTGCGGCCATGGCGCGGCGC |
| | | TGACTGGCCTGGCCCGGCCCCGCCGCGCTCCCGCTCGCCCCGACCCGCACTCGGGCCCGCCCGGGCTCCGGCCTGCCGCCGC |
| | | CTCTTCCTTCTCCAGCCGGCAGGCCCGCGCCGCTTAGGAGGGAGAGCCCACCCGCGCCAGGAGGCCGAACGCGGACTCGCCA |
| | | CCCGGGTAAGCCAAACTCGGGCGAGTGGGGGCCCGGCAGGGGACGCGTGGCCCAAGTCCTGCCGCCCAGCCCTCCGCACCCC |
| | | CTCCAGCCCCACTCGGACTTCCTCATTCCTGCGCTAACCGCTGGGCTAGACCGTGGGAGGAGGTTGACAGTAGCTGAGAGGC |
| | | ACATGGGATTAGCGACAGCGGGGAAAGACACATCCGGACCTCGCAGGGGCTAGTCGGCCGAAGGGCCGCGGCCGCCCGGCGG |
| | | TCATTTCTCTTCACGTCCCTCCGCGGGGCGGGAACGCGAACCGAAGGGGAACTCTTACAAACTTTTAAAATCCCCAACCCCA |
| | | GCCCCACCTGGGGGATGGTGAGAAGGTTGGAGTGCGCTGCGGCGCGGAGGAGAGAGGGAAGGTGGTTGGTGCAGTGCAGAAC |
| | | CAGATTTCACCTAAGGGCGTTCCATGAAATGAAAGCAGAAGTGCTTGTCAGTCCTCTTGGCTGGGAAAGCCCTCCCCAG |
| 506. | NFKBIB | TCAGTGGGAATTTCCAGCCAGGAAGTGAGAGAGTGAGCGAGACAGAAAGAGAGAGAAGTGCACC |
| | | AGCGAGCCGGGGCAGGAAGAGGAGGTTTCGCCACCGGAGCGGCCCGGCGACGCGCTGACAGCTTCCCCTGCCCTTCCCGTCG |
| | | GTCGGGCCGCCAGCCGCCGCAGCCCTCGGCCTGCACGCAGCCACCGGCCCCGCTCCCGGAGCCCAGCGCCGCCGAGGCCGCA |
| | | GCCGCCCGGCCAGTAAGGCGGCGCCGCCGCCCGGCCACCGCGCGCCCTGCGCTTCCCTCCGCCCGCGCTGCGGCCATGGCGC |
| | | GGCGCTGACTGGCCTGGCCCGGCCCCGCCGCGCTCCCGCTCGCCCCGACCCGCACTCGGGCCCGCCCGGGCTCCGGCCTGCC |
| | | GCCGCCTCTTCCTTCTCCAGCCGGCAGGCCCGCGCCGCTTAGGAGGGAGAGCCCACCCGCGCCAGGAGGCCGAACGCGGACT |
| | | CGCCACCCGGGTAAGCCAAACTCGGGCGAGTGGGGGCCCGGCAGGGGACGCGTGGCCCAAGTCCTGCCGCCCAGCCCTCCGC |
| | | ACCCCCTCCAGCCCCACTCGGACTTCCTCATTCCTGCGCTAACCGCTGGGCTAGACCGTGGGAGGAGGTTGACAGTAGCTGA |
| | | GAGG |
| 507. | Notch4 | CCCAGGGCCACCCAATCACAGGGCCAGTCATCCGTTGAGACCCTGCCTCCGCGCCCGGCAGCCA |
| | | CTCCGTATCTTCCTCGCATTATCGCAGGGTTGGGCCGAGGCCCGCGCATGCCTGCAGAAAACCTACGGCCGCGAGGGGTCGG |
| | | GCCTCCTCCTGCTCCTACTCCCGAGAGGCTCCGGCAATGAGAATAGGCCCCGCCCCCCCGCGCAGCCAAGTCTACGGACCAA |
| | | GTCCGAGCCTGCAGACAAGCTCCGCCCCCACGAGGGCCTGCTCCGGCTGACAGCGTCCGGCAGCGCGGCAGAGCCCCGCCCC |
| | | CATGCGGGGGCACGCTTACTGACACCGTCCGTGCGCGCGGGAAGGGCCCAGCCTCGCGGCCCGGCGTGGCTTTGTGACGGGC |
| | | CTCTGGTGGCCCAGCCCCTTC |
| 508. | NR2F2 | ATGTCATATATATATGTCACCAAACCGAACAAGTGATCAGAGTTAATTAAGAGAAGGGTTTGCC |
| | | ATCACCCGGATCTGGCCAACTCTTTCGTTTGGAAAATGCCTACAAGCTCAGTTCGTGGCACCTTCCCACCCTCACTCCCTAT |
| | | CCCTCTGCAAAGAAACCAGGGAACTTTTCTTTGGCATTGTGGGTACTAAGATGCGCCAGGAGTCCCGGGTGGTGCGCGAGCG |
| | | GCGGAATTCGTGACATCGCCAAGTTGCGAGAGGCAAACTCCCCGATTCCATCCAAAGCCTCTCGCGGAGCCTTTAAGAGACG |
| | | TTGTGTGTGCGCGCTGCGCTAGTCTCCCCGCCGGGGCGGAAACCTCGGGTGCGGGATGTGGGGAGAGCAGAGGCAGCGCTCG |
| | | CAGGGCCAGCACGGGCCCATCCCCCTCTCGGCGGCGGCGGCGGCGGCGGCGCGGCCACCCCGTCCCCGCCCCCCCAACCCCC |
| | | GGTCCCCCGCGTGCCCCGCGCCGCGCCGGCGACGCCGCTCGCGCTAGGACCGGGCTGCGCCCGCGGCCGCCATGGCGGGGCC |
| | | GCCGCAGTCCGGCCAATGACGGCGAGGGGGCGGCGCGCGCGCGCCTGGCCAGGCCAACCCCGGCTGCTGCCTTATAAGGCGC |
| | | GCCGTCGCCATGGCAACGTGCGCTAAGTTGCAGCAGTCGTGTCAAAGTTCACTATATAGAGAGCTCAGTGAGCTGATCGCGG |
| | | AGAAGCCACTTCTGCCAGCCCCGGCGCCTATAAATCGCATTCCCTCCCGCGCCCCCCTTTTTAGCATATTTGATCACTTTGA |
| | | TTCTCTGTTCTTTTCTCTCCGCGGTGTGTGTGTGCGTGCGCGCGTGTGTGTTTTCTTCTTCTCCTCCTCCTCTCCCCGAGTT |
| | | GCCTCCTTTCTCCGGGTGCCGTACTGCCTTTTTTCCCCTCTTTCATTCTTTCTCTCCGTCTTTTTCTCCCCCCTCTGCGCAC |
| | | GAAGGATGTGCTTCTAGGTGGTGATCTGCCCTCCTCTCTCTCTTTTATCATTTCTCCCCCGCCGCCGGCGAGTTGACTCTTT |
| | | CCCTATGTGTGTGAGGCGGCGGCGGCAGCAGCAGCAGCAGCGGCTCCGGCGGCGGCAGCAGCGGCAGCAGCGACTTCAGCGG |
| | | CGGCGGCGGCGCTAGACGCAGCGGCTCCGGGCCCGACCCGGCGGCTTCGGCGGCGGCTCCGGCGGCAGCGGCGGCCCGGGCG |
| | | GCCCGCAGGGAACGGCGAGCGGCCTCCACCCAGCGACTGCGGGCGGCGGCGGCCGGAGAGAGCGAGGCGCGCGCCGGACGCC |
| | | CGGGGCAGGCGGCGGCGGCGGCGGCCCAGCGCCAGGACGACGCCGCGCAGCGCCCGACGCGGACCACTTTCATGCTGATTCC |
| | | CCCGGACCCGGGCAGCGCTCCGGCCACTCCGCGGGCCGCCGGCCTCCGCCCCGGCCTGCCTGGCTCCCTGGGCGCGCCCGCA |
| | | CCCGGCGCCTCCGATCTCCTAGTCCTCCTGATTTCGATGGCTTTCCTGAATGGCTGACTGTGGGCTGCCCTGGACTTGGCCC |
| | | CCGGACAGTCGCCTCTCCTCCTCCTCTACCTCCTCCTTCACCACCACCTCCTCTTCCTCCTCCTCCTCCTCCTCCTCCTCCG |
| | | CCAACTCCTCGGCTGCACACCAGCTCTAAGAGCGAGAGTGAACGAGAGAGGGAGGGAGAGAGTGAGAGCGAGCGAGATCTTT |
| | | GGAGAGATTTTTTTTTTTGCCTCCTACTTCTGTCTTGAAGCCAGACAATCGACTTCAGCTCTCCCTCCCCTCCCTCTTTCTC |
| | | CACGTTCTGCTCCCACTCGCTCTCCTGTCCCCTTCCCCTCCCCTCCCGGCGGAAAGCCCCCCGAAACCAACAAAGCTGAGCC |
| | | GAGAGAAACAACAAAACAAACACACCGGGCCAGACAAGCCATCGACAAAACTTTGCAAAAGCAAAAACAAAAAAGGAAAAAC |
| | | TAACCAACCTCAACCAACCAGCCCCCGAGCCACCCGGGGCGCCCTCCCGCGCCCTCTTGCACCCTCGCACACACAAAAGGCG |
| | | GCGCGCCGGAGCCCGAGACCCGGGGAGCCGCCGCCGCCCCGCCGCCGCCCGCAGCCAGGGGAGCAGGAAGTCCGGACGCAGC |
| | | CCCCATAGATATGGCAATGGTAGTCAGCACGTGGCGCGACCCCCAGGACGAGGTGCCCGGCTCACAGGGCAGCCAGGCCTCG |
| | | CAGGCGCCGCCCGTGCCCGGCCCGCCGCCCGGCGCCCCGCACACGCCACAGACGCCCGGCCAAGGGGGCCCAGCCAGCACGC |
| | | CAGCCCAGACGGCGGCCGGTGGCCAGGGCGGCCCTGGCGGCCCGGGTAGCGACAAGCAGCAGCAGCAGCAACACATCGAGTG |
| | | CGTGGTGTGCGGAGACAAGTCGAGCGGCAAGCACTACGGCCAGTTCACGTGCGAGGGCTGCAAGAGCTTCTTCAAGCGCAGC |
| | | GTGCGGAGGAACCTGAGCTACACGTGCCGCGCCAACCGGAACTGTCCCATCGACCAGCACCATCGCAACCAGTGCCAGTACT |
| | | GCCGCCTCAAAAAGTGCCTCAAAGTGGGCATGAGACGGGAAGGTATCGGCCTCTCATTTCTCCTTCCCTCGTCCTGGGTCCC |
| | | GGGGTCCTGGGTACGTTTGGCTAGCCTGCTCTGGGTAAGGACAAGAAGCCCCAAGCTCTTCTCTTCGTATTGCAGCGGAAAA |
| | | GGGTTTTATACTAGAAGCGAGTTCTGCATTGGAACCCAGACCCCAAATCCGCATGCTTTGGCCGACTGATTTCCTTCTTTAC |
| | | TCTCTCTTTGGGCTGTTTCCATTTCCTTTGCATTGATTGTGAGTTCACTGGAGTCTGCCTTTCTGCAAGGGATGGGGTGTTT |
| | | GTTGTTGTTGTTTTAAAGCCTAGTTTACTTCTCTCTCTCTGCCCTTGTTTTTCCTGCATGTTCAACATGTCCCTCCCCCTCA |
| | | CCCCTTTCCCCAGCCCCCACCCTCTCAAAAAAAAAAAAAAAAAAACCTGAGATTGTACTTTTGTACAGGAGGTTCAAATTAC |
| | | AAATGGCAATTTTATGCACTTCGCCGTATTAACGCTGCCGCCCGGGCAGCGCTCATGTGACCCTCCGTGGATTAACATCCTG |
| | | CTAAAAAAAAAAATACCTCTGCTTTCTTTTTTCCTTTCACTTTTTGAAACGAAGAGAGCGCGATAGGAAGTAGGAAAGGGTG |
| | | GGCGAGGGGCCCTGGGCGGCTGCTTTCGCTCTGCGCGAGTTGGGTCTTTGTGATATAAAATTCGCCGAGCGCCGCGAGCCGT |
| | | GCTTTGCCAATGGCGCGCTCGGCGCGGGGCGCGGGCTCCGGGTTGGGGCGAGCCAACGCCGGGGTTTCTTTGTGTTTCTGCG |
| | | AGAGCGACTCTCCCGGTCCGGAGTCAGATAACAGCCTGGGCCCGAGCCTCGCCGGCTTTCCCCGGCCCTTACAGGCCCTGCC |
| | | CAGGCTCCGCTAGTGCCGGCCGCCTGCTCCCTGCCTCTCCCGGCTTCCTCTCTCTTTAGCCGGCCTCTCTCTCTCCGCCCTC |
| | | TCCCTCCGTCTCTTTCTCCGAGCACACTGATTAGACAGACGCCAGACCTCCGCTCTCTGCTTGTCTCTCACTGGGGGGGTTC |
| | | CCCGCCGGGCTGGGGCTGGGGCTTCGGGGTTTGTGGGAGAGTCGTTCCGGAGTGGCCACAGGCCGTCTGGGGTGGACCCTCG |
| | | TGCCTTTTGCAAAAGCGCCTCACCCTCCCCCCAGACTCGCCCCTCCCGCTCCCTCTCCTCCAATCAATAAGAAATATCAGCT |
| | | GTTTAGCAGTAAAGAAGAAAGATGCCCTCAGAATGCTACATCCCGCCCACAGCGCCGGGGACCCCGAGGCAAGGTGGCCAAT |
| | | TCTGGGTCCTCGGCGGACCAGCCCCGAGCGGGCCTCGGAGGCAAGTGTGCCCCTCTGGCCCTCAGAGCTCGCCTGGGTGGTG |
| | | GTTTGAAAGGAATGGTGCCAAGAGCCTGGCGACTCCAGCTCTGTGGCAGGCCTGCCTGGTTCTTGCGCTGCTCAGGGCCTGG |
| | | GTCAGGTGGGGGTCGGGCTGGGGCAGACCCCGCCGGGGAACCTGGGGACTGAGGCTGGTCATTAACTGTGGAGTGTCTCCTT |
| | | TCCTCCCCGCAGCGGTGCAGAGGGGCAGGATGCCGCCGACCCAGCCGACCCACGGGCAGTTCGCGCTGACCAACGGGGATCC |
| | | CCTCAACTGCCACTCGTACCTGTCCGGATATATTTCCCTGCTGTTGCGCGCGGAGCCCTATCCCACGTCGCGCTTCGGCAGC |
| | | CAATGCATGCAGCCCAACAACATCATGGGTATCGAGAACATTTGCGAACTGGCCGCGAGGATGCTCTTCAGCGCCGTCGAGT |
| | | GGGCCCGGAACATCCCCTTCTTCCCCGACCTGCAGATCACGGACCAGGTGGCCCTGCTTCGCCTCACCTGGAGCGAGCTGTT |
| | | TGTGTTGAATGCGGCGCAGTGCTCCATGCCCCTCCACGTCGCCCCGCTCCTGGCCGCCGCCGGCCTGCATGCTTCGCCCATG |
| | | TCCGCCGACCGGGTGGTCGCCTTTATGGACCACATACGGATCTTCCAAGAGCAAGTGGAGAAGCTCAAGGCGCTGCACGTTG |
| | | ACTCAGCCGAGTACAGCTGCCTCAAGGCCATAGTCCTGTTCACCTCAGGTAGGAAGGAGCCCTGTCTTCTCGTGCCCACGGG |
| | | CTCCTAGCCCAGAGCTGGGGCCCAGAGAACTTGGGAGTCCCCAGGGCAAACCCAG |
| 509. | NRP1 | TGCCTTTGGGAGGAAACATTATTAAGATAATTGGCATGCTGATCTGGGAAGGTAGACAGCTCTG |
| | | TCTTGTAACAGAGGTAAGGAAAAGCCCCACAGCCCAAGGATCAACGTCAGGAGGGAGCCCAAAGACCTGAAATCCTCCTGCT |
| | | CTAAACCGCCTGATCTCATTTCTTCTTCTCTCCTCCCAGATAAAAGTTTCCTTCGCCCGGGAGTCGGTTGTTCCCGGCTGAT |
| | | CCCGGGAAGCCCCGCCTGAGCCCCGGTCCGGGGCGGGCAGCTGGGAGCCGGGGCGCCGCTGTCACCCGCGCCTCTGCCTGTC |
| | | ACTTACCGTTGCGAAAAGCGCCGGCCGGGGCGAGGACGAGGGCGAGCACGGCGCAGAGGAGCGGCAGCCCCCTCTCCATTCT |
| | | CCCTTCTCCGGGTCCGCAGGCAGACGCGGGAGAACGAGGACGTGGGGGGAAATGCAGCAAAGAGGAGAATCTAAGCGATCCG |
| | | AAGAGCCCCAACTCCGCCTAGAGCTGTACAATCCTCAGCCCGTCTTGGAGAAAAGAAAGCAGCGAGGCAATGCCTGGATCCG |
| | | AGAGGAACGCTTCTCTTTTTGTGTCTCAAGTCGCCTGCATCCTGTCATTTAGCTCCGGCTTCCTCTCCCTTTTCCCACACTT |
| | | GTTCCTCTTCCAGGAGCCACTGCCCGGGCCATGTCTCAAAAAAAAAAAAAAAAAAAAAAAAGGCCGGGGGGGGGCTGTGGGT |
| | | GGGAGGGGGAGGAGGGAGGTAGGGCAACACACACCAAAGCCAATTTCCAGGAAGAAAAAAAAAAAATCCGGCTTGTTTCTGG |
| | | ACCCGTTGGAGCCGCGGAGCTGGCGCCCAGGGGAGGTCCGGGTGTCTGCGGGAAGGAGGGGAACGAGCAATGAAAAGATGAG |
| | | CGGGAGACAGAGGAGTTTCACCAACTGCACCCCCGCCTCCCCTCCTGCGGCCTCCCCCCACTCCGGAGCCCTTCCCTCGCTT |
| | | CCTCCCGCCCCTCTCCCCGCTTGGGCTCGCCTGTAATTGCCTGACAGGAGACTGGGAGGAACAACTTTCCCTCCGAGTGCTC |
| | | TCCCCGTCCGTCTGTCTGTCTTTCCCAGAGACCCTGCAGGGATCTCCGCGGGAAGGAAGGCGCTGGGAGTCTGCGCCCCGGT |
| | | CGCGTGGGTGCGGGCGTCGGAGGAAGGGGCGCGGAGGTCAGTGCCAGCCGCAGGGTCTGGCCAGCCCACCTCGCGTGCTCGC |
| | | TCTGCACCCCTAGAGGGAGAGCTGGGCAGCTCCTGCAGGCGCAGATCCCGCCGCCGCGCAGTGCGGAGCGCCCGGGAGGTGG |
| | | CGGCTGCTTCTCGCAACTTCAAATCCCGGGGCCCGCGCCCTCGGCGGCGAGGAACGCAGGGTCACCCCCTGCCACTCCTCAT |
| | | CGCGCCATCCTGAGACCTGTGGCGGGGAGCAGCGGGGCACCCCTGGACGCCCCTGCCCAGAGCTGGAGGGCGAGGAGGGGAA |
| | | AGCCGGGCTGGAGTGGGAGCCGCACCGCGAAGCCGGGCGAGGGGCAGCAGTGTCGCTGTGGTGTGAAGCGAAGACAGACCGC |
| | | CTGGGCGGGCGAGGCCGGGCTGCAGCCCCACGCGGCCTCCCTGGCCCGCATCAGGGCGCGCGGCTGGGTGCCCTCTGCGCGC |
| | | CGCGGAGGCTTCATTGTTTCCCGAGATTTGGTGCCCGTTCCTCTGGGGCAGAGGGAACACCGGAGGGTGGGAGGTGCTGCGC |
| | | CTTCCAAGCAGGGGGGCAATCGCCGCCCGACCCCGCCCAACCTTGTCTGGGAGCCCAGGTGCTAGGGAGGGGTGTGGAAATC |
| | | AGGGGTCGGGCGGCCCCCGTTTCTCTGTGCATCCCGAGGGAGGGAAAGAGTGGCGGGGGAGGGGTGCTCTTCGCTGCGCCCT |
| | | CTCAGTGTCCGCTCCCGGCCACAGACAACGTGCACGCCGGCCCTGGAGCTCCTGCCACCCCCACTTCCCACAGCGGCTCGTG |
| | | CCCCCAGCCCGCTGGATGCCCCCCAGGCCCGTCCCCGAGGGTTTTCCGAGAAGTGCTGCAGGAAGCGTTTGTAGATGCGAAA |
| | | AGCAAGGACATTTCTGGGAAGAAACAGGTTGCGGTCACCGATCAGGCCCCGGAGAAGGGTCAGTGCCCACGGGGCACCTCCC |
| | | AGGGAGTTTGTGGGTAAGGTGAAAAGGTCACACGCCTCCTGGGGATTGGCGTCTACACACACAGCCAAAGTACAGCCTGATT |
| | | CCTACCTGGCACAAATAGCTGGCCAAAGGGAGGTAGGAATGCTGGCAAGGAGACTCTGGTCAAAGGTTTTGGCGGAAGAAAA |
| | | AGGGGCGGGCAGGGAGAAAGCCCAAGGCGCGCGGGCCCCAGGCCAGGACTGCGCGCTCGCGACTCGGGCTGGGAGCAGTGAC |
| | | GTGCGCCCCTCGGGGTGCAGGCCGTTCCCGGGCCAGTGGGAGGTGCACCCACGGAGCCCGCACTCGCCACTCTCTTGCCCCG |
| | | GGTCAAGCCTGTCTTCTCCTCCCCACGCGCCTCGGGATTCCCAGTCGCCAAGTTGGAAACTCGGCCCCCCCGAGCGCCTCTT |
| | | TCCTCTGCCAGTTTCCTCCTTCTTCTCAGCTGTCGGACCCCTGGGAGGCGCGGGGTGGGAAGTGGGCACCGAAGGGCTGGGA |
| | | GTTAAATGCCTAAGTTGAGCGGAAGGAGCACTGTCGAAGCCATGGGCAGCCCTGCGCCCGGGCAGGCGGGCAGGCGGGAGGC |
| | | GAGGCGGGGGCGCCGACCGCGCCCAGCGCAGACCCCGGGGGAGTGAGCGCCGCAGAGGCAGCCTGGGCTCTCGGCTACTACC |
| | | CGCGCGCGGTCGCCGCCTCCCCAGGTGCAGCCCCGCCAGTCCCAGCCCCCGGCTGCGCCGGGCGAGCGGCATCATCAGATTT |
| | | AGGGTGACCCGGAGCGACCTCGGTCCAGGCTCGGAGGGAGAGGCGCCTCTCAGGACCTGGCAGGTTGACAGGGAAGGGGAAC |
| | | CGGGAGGGAGCGGCGGCCGGGGCGGCCTGGAGTCTCGGAGGCCCGCCTGCGTCCTGGGGGGACCCGACCTCCGCGCTCGCTC |
| | | CGGAGCGCCCGTTTGGATAGCTCTGAGCCTCGGCGGGGTGAGCGAGGGGGCACACGAGGGAGACTTGAGCTGTGTTGTTTTT |
| | | AGAAAACTGCCGCCAATCTCTCTTCCCCTTTCCCTGCTGAGTGTGCATTTTCCAGCCTAGACCCGCCAGGCCAGCAAATAAA |
| | | GCCACCGGATCGTGCACTCCGGAGCGACTGCCCCCACCCCGACCTTGTCTTTCCCGAAGGGAATGGCTCCTCCGGCGCCTCA |
| | | ACGCACAGAAAACGCCTGCCTCCAGCTGAGCGCTTTTGCCAGCTTTCTCCAACCAGTGAATAAGGGATGCACTATGGACTCA |
| | | GAAGGAAATGTCATATTTAAGTTCTGCCTCTGATTTCTCTGCTCTTAGCTGATCTAAGGTTTTTCTAGACTCCTTAGTTTCC |
| | | ACTCACAGTGCTGGGTAATGGAGCCCTACCGCACTGATTGAGAATTGCCG |
| 510. | p16 | TTTTGTATCTGATAAAGAGCATACTTCCATCTAATACAAATATGTTCCCCCCTTCAGATCTTCT |
| | | CAGCATTCGAGAGATCTGTACGCGCGTGGCTCCTCATTCCTCTTCCTTGGCTTCCCAAGCCCCCAGGGCGTCGCCAGGAGGA |
| | | GGTCTGTGATTACAAACCCCTTCTGAAAACTCCCCAGGAAGCCTCCCCTTTTTCCGGAGAATCGAAGCGCTACCTGATTCCA |
| | | ATTCCCCTGCAAACTTCGTCCTCCAGAGTCGCCCGCCATCCCCTGCTCCCGCTGCAGACCCTCTACCCACCTGGATCGGCCT |
| | | CCGACCGTAACTATTCGGTGCGTTGGGCAGCGCCCCCGCCTCCAGCAGCGCCCGCACCTCCTCTACCCGACCCCGGGCCGCG |
| | | GCCGTGGCCAGCCAGTCAGCCGAAGGCTCCATGCTGCTCCCCGCCGCCGGCTCCATGCTGCTCCCCGCCGCCCGCTGCCTGC |
| | | TCTCCCCCTCTCCGCAGCCGCCGAGCGCACGCGGTCCGCCCCACCCTCTGGTGACCAGCCAGCCCCTCCTCTTTCTTCCTCC |
| | | GGTGCTGGCGGAAGAGCCCCCTCCGACCCTGTCCCTCAAATCCTCTGGAGGGACCGCGGTATCTTTCCAGGCAAGGGGACGC |
| | | CGTGAGCGAGTGCTCGGAGGAGGTGCTATTAACTCCGAGCACTTAGCGAATGTGGCACCCCTGAAGTCGCCCCAGGTTGGGT |
| | | CTCCCCCGGGGGCACCAGCCGGAAGCAGCCCTCGCCAGAGCCAGCGTTGGCAAGGAAGGAGGACTGGGCTCCTCCCCACCTG |
| | | CCCCCCACACCGCCCTCCGGCCTCCCTGCTCCCAGCCGCGCTCCCCCGCCTGCCAGCAAAGGCGTGTTTGAGTGCGTTCACT |
| | | CTGTTAAAAAGAAATCCGCCCCCGCCCCGTTTCCTTCCTCCGCGATACAACCTTCCTAACTGCCAAATTGAATCGGGGTGTT |
| | | TGGTGTCATAGGGAAAGTATGGCTTCTTCTTTTAATCATAAGAAAAAGCAAAACTATTTTCCTAGTTGTGAGAGCCCCACCG |
| | | AGAATCGAAATCACCTGTACGACTAGAAAGTGTCCCCCTACCCCCTCAACCCTTGATTTTCAGGAGCGCGGGGTTCAC |
| 511. | p53 | TGTTCATTGCTTTCAGTACATGGAAACGTAAGCCTTATGAGGATATAGAATTTTTCTACTATCT |
| | | TATTCATTGTTGTATTCCTGAGTGCCTATATCAGTGCTGGGTAGCAAGTAAGAGCTCGATAATAAATATTTTTTGAATGAGG |
| | | GAGACAGGTCTGAAGCCTGGAGAATGAGATGCAGAAGAGGTGCAAGACCTGCTGCGCCCTCTGCAGGCGGCGGGGGGGCGGT |
| | | GCAGGTGCTTTAAGAATTACCGCGGGACTCGGTAGGGGGAGCGTAGGCGCTTCTCGCCAAGATAGAAGCGTTCAGACTACAA |
| | | CTCCCAGCAGCCACGAGGAGCCCTAGGGCTTGATGGGAACGGGAAACCTTCTAACCTTTCACGTCCCGGCTCCGCGGGTTCC |
| | | GTGGGTCGCCCGCGAAATCTGATCCGGGATGCGGCGGCCCAATCGGAAGGTGGACCGAAATCCCGCGACAGCAAGAGGCCCG |
| | | TAGCGACCCGCGGTGCTAAGGAACACAGTGCTTTCAAAAGAATTGGCGTCCGCTGTTCGCCTCTCCTCCCGGGAGTCTTCTG |
| | | CCTACTCCCAGAAGAGGAGGGAAGCACAGGTGGGTTTCTTTAGCTCTGCGTCGGATCCCTGAGAACTTCGAAGCCATCCTGG |
| | | CTGAGGCTAATCTCCGCTGTGCTTCCTCTGCAGTATGAAGACTTTGGAGACTCAACCGTTAGCTCCGGACTGCTGTCCTTCA |
| | | GACCAGGACCCAGCTCCAGCCCATC |
| 512. | PAGE5 | TGGAGTGGGTAAGATCATTGCAAGCATGACAGCAGACTCGCGAAGGCACAAGAAAATGATTTAT |
| | | GAAAGCAGATATAAAATGGGTATTAACATATATAATATTTATTTCTATGATGGCGAATTTGTCCGAAACACATTGTTTTCTT |
| | | AGAGTAACCTGGGCCTCATATTCTCCAACGCCATTGGAAGTGAAAGGAAGGTTGGGCGGGGGAAAAAGAAAAGTGCTTTGTA |
| | | AGTTCATTTTCGTTGTCAGAAGAGCTTGTGGTTTAGGTTCTCCACAGAGGCAGGAAACCTTCAGTCACGTGGCAAATACCCC |
| | | ACTGAGTGCGCATGCTGCACAGATCTGTTGCTGCGCATGCGCCTTTCTGCCCACCTTCTGTCTAGGCAGAGCTCTGCAAGGA |
| | | GAGGTTGTGTCTTCGTTCTTTCCGCCATCTTCGTTCTTTCCAACATCTTCGTTCTTTCTCACTGACCGAGACTCAGCCGGTA |
| | | GGTCTGCAGAGTGGTCTTCCTGGTA |
| 513. | PBX3 | TGTGTAAAGTGTTGTAAACCTTGAAATTAGTCCTGGACTGAAGGCACTTTCCCTCCTCCCAGAC |
| | | TCGCTTCTCTCTGACACATTTCCCCTTTCAATGGAAAGTCTATGGCAGTCAGGTCCACTCAGCTCAGACTTCAAGTATGTGA |
| | | GACCACAGGTTCCCACCTTAACTGGTCACCCATTGAGGGACAGCACAAGACCCCGGTGCTGGCGCCAACGCTGAGCTGCGCC |
| | | CTCTCGGTTACCATGGCGACCGCAGGCGGGGCGAGGCCCCCACTCGCCGCTTCCCCGCCTCTGCCCAGAACCTTTCGAACGC |
| | | TCCAATTGGGACGACCCTTTCTTGCCTCTGTCCCGCCCCATTCGGCTCTAAGCGCTTTGCGATTGGCCCGGGACGAGCGGCC |
| | | TGGTACCCAGAGGGCACCACCTCCTGACAGGAAGAGCGGGAAAGTGCACCAGGAGCTGTCCGAGGCGTGAAGCTCACGGGCG |
| | | CGGCGGGGGAGCACACGGTTGGCTTTGCAGCGAGTTGCCCACTGTCCCATACACAACCCCCCGCTCCCATGACTGCCAAAAG |
| | | CATGGAGCAGGAGTCGGGGTGCGGGCACACCGTTTGTGGGCCGGCCCGGTCTATCCCGGGGGGCTAGGACGCGACGAGGTTG |
| | | GAGCGAGGATCCGGCGGCACACTCCGGAGCGCGGAGTATCAGAGGCCAGAGGGAACGGGCCGGGCCCCGGGGGCGGTGCCGG |
| | | CGGCGGCGGCGGGAGGCGCAGAGCCCAATGAGCGCGCGCACCGCCCCGGGGGCGGGGTCTGAGGAGCGCTCCCCCGCCCTTG |
| | | CCAGCCCCCGTCCCTCCCCCCGGCGCGGGGCCGCAGTGAGTGAGTGGCACTGGCAGCCTGTCAATCCCTCGGCCCAGCGGCC |
| | | TTCCAGCCCGGTCCGCGGCGGCTGCTGGCTGGGTGCGCGGCTCCGGCGGCGGCGGCGGCGACTTCTCCCGCCCTATCCATCG |
| | | GCTGTCCGCCCGGCGCGCGGCCCGCCGGGGCCCTCCTCCGGGCTCAGCGCCCCCGCCGCCTCTGCCCGCCCGCTCCCAAACT |
| | | TTCCTCCTCCGCCCCCCCTCGCTCCCCGCGGCCCGCCCGCAGGCCTCCTCCTCCGCCGCCGCCCGCCGCCGCCGCCGCTGCC |
| | | TCCCCCGGGGCCGCCGGGGCTCGGATCCCGCCTGGCCGAGGCGGCGGCGGCGGCCGAGGAGGGCAGTGCGCAGGCTGGCGCG |
| | | GGGGGCGGCGGGCGTCCCGGCAACTCGGCGGGCGCTGAGGAGCAAGTTGCGCGGGGCCGCCCGGCGGGGCGCGCGGCGGCGA |
| | | GGAGGCGGCGCGGCGGCCGCGTGAGGGCAGCGGGCGCCGCCGCCTCCGCCTCCGCCACCGCCGCGGAGCCCGGCGCTTCCGC |
| | | CCGCCGCTTCTCCTCAGCCTCGGCGGCGGCGGAAGCCGGAGCCGGGCGCCCGTCCTCATCGCCTCAGCCGCGGGCCCTGCCG |
| | | GCCGGGCCGCCCCCTCCCCGCGCGGGCGGGGAGCGCGCGGCCGCCTCCCCCTCCCCCTCCCCCTCTTTCTTCTCCTCCCTCG |
| | | TCGCCGCCGCCGCCGCCGCCGCCTCAGCCTTCGCCTCAGCCGCCGCCCGCTCCCGCCCGCGCGCGGCGGGATGGACGATCAA |
| | | TCCAGGATGCTGCAGACTCTGGCCGGGGTGAACCTGGCTGGCCACTCGGTGCAGGGGGGCATGGCCCTGCCGCCTCCCCCGC |
| | | ACGGCCACGAAGGGGCGGACGGCGACGGCAGGAAGCAGGACATCGGCGACATCCTCCACCAGATCATGACCATCACCGACCA |
| | | GAGCTTGGACGAGGCGCAAGCAAAGTTGGTGTCGTCTCATTAAGCATCTTTTGTGTGTGTGCGGGAGCCGGGCCCGCGGCCG |
| | | AGTCGAGGCCCGGGGTGGCGCCCGGGGCTAGGGCCGCAGCCCCCGGCGGGGAACTTTCTCCGAAAGCCGGCCGCCCGCCCCG |
| | | GCCTCGGGGGGACTTGCCCGCGCCCCCCGAAGCGGGCGGGAGTCGGCAAAGTTGCTCTGGGGGGCTCGGGACGGACTCGGTG |
| | | CGGCGCGGATTCCGTGGCGTCCTTTCCCCCGTCCTGCCCCTCGCAGCCCGGCCCCCTCCCAGGGATTTAAACCTCCCGCCCC |
| | | GACCCCGTGCGGGCCGCGCCGCCGGGAAGTGTAACTTTCTCCTCCGGCGGGGAGTCCCGGCGCCGGCTCCCGCGGCCGCGGG |
| | | ACGACCTCGCGATGCGGGCGGCCACCCGGGCCCGGCGCGGGGCGATGGGCGGTTCCCTGGCGGGTCCGGGTGCGGACGGCCA |
| | | AGTTCTCGGAGAGGGAGGGCCGCCTTGCAAACTTTGCCGAGCTGTCACCCTCCCGCTGGCCGCAGTCGGCCGGCCTCCTCTG |
| | | CACAGGAGCGGACGCGGGAGCCCCTCCGCACCCGTCCCCTCCCCCGGGTCGCCTTCGCCTGCCCCCGGGGCGAGGCTCCCCG |
| | | CGCGGGTTCGCGTCGCGTCTGCAGTGGCCGAGGCTGCTGCCTGCCGGGCAGATGGGTCCGCCTTGTTCCGGCTGCAGCTTTC |
| | | GCCGCCGGGGCTTGCTGGCTGTCGGGAACTAGTCAACTGGAGTTTTTGTTGACTTCCCACGGTTCAAAAGGGCCTTCCAGAA |
| | | TGGGGGGCTGGAATTAAATCTTGGGTCTAACTTAAAGGAAGGCGCCATATTATTCCATAGGTGATGCTAATACCTTTGTGTT |
| | | TCGTTATTTGTTTTTTAGGAAACATGCCCTGAACTGTCACAGAATGAAACCAGCGCTCTT |
| 514. | PHEMX | ACAAAGCTGGGTTCCTGCTGGGCCCCGCCCTGCTCCTCGCCCCCGCGACTGGGCTGGGCGCGCT |
| | | GTCCCCTAGCGCAGCTATGTCCCGAGCGCGCCCCCACCTGTGCGTTAATCTACTGGGAATGGGGGTGGACTGCGCCTTACCT |
| | | GGGGCGGGGTGGGGCTTAAGGAGTGGTCGAGACTGAGGCGGGGTGGGAGGTTCAGGTTCCCGGGGCGCCTTCCCCAACCCGC |
| | | CCCGCTTTCCCCGTCCCTCCACGCGCACCCTGCCTGTGGTTTCCGTGCGCCCCCGGCCTGAGGGCTCTGGGCGGCACCTTAA |
| | | CCCGGAGGGCCTGGAGGTCTGCACCCGACCGCCTTGTGCCAGGACGGTCAGGTCCACGCCCTCCCCCACCGTGGCTCCCTCC |
| | | ATCTGCAGTATCCCCCACCTCCAGCCCGTCCTGCCCTCCTGTTCTCCGTCTCGCTTCCCGTCGGTGCCTCCGGGATCTCACA |
| | | GCCCTCGCACCTCTTTTGTGACCCAGGCTGTTTTTCTGCACCCCCCTCTCCCCTGAGGGCACTGAGATTGGGCCATTGGCCT |
| | | GAAGGTCTCTGGGAGCAGCACCCTTCCAGGGGAGGTGG |
| 515. | PIK3R4 | GGGTCTTTCTTCATAGACAGGAGATGGGCTGTTGGTGGAGGGGCGCAGAAAAGTCCCGATCTTC |
| | | AGGAACCCCGGTGGTCCTGGGAGGAGAACTGGGAAAGCTCTCGGGGTCTCAGCAGAGAGAAGAAGCCACGCTAAAGAGTCTC |
| | | CACAAGTACCCCGGGATTTGACCCCTCCAGCCCCCTAACCTGCCCTGGGGGACATGCGTTCCCGGGCCTCGTCTTCCTCTAG |
| | | CGCGGATCGCTACACCCGTCCGAGCCCTCCTCGAGGGCCTCACCACCGGGCGGGGGGAGATGGAACCCGGGAGAGAAGTGCA |
| | | GACCGCCAGTCCCAAGAAAACACTACACTTCAGCTGCTGCAGCCCCAGCAAACGCCGAACTCCCGGGAAAGCAACCGGTCTG |
| | | ACCTCACTTCCTGTCGCCGAGCGGAAGTGACGAAAAAGAGTGAGCCTTGAGGAATTGCCAGGGTAGGAAACCGGTTGCTAGG |
| | | AAACTAGGGAAGGCGTGGTCTTACTTTGGGCGAGTCTACAGGGAGAAAATGATGCTCTGACAAAGATCTATTCCCCTTCCTG |
| | | CTCTTCTACCTTTCATTCACGAACGCTGAGGTTTCTGACATCCCCTAGAATCTGCTCAGACACGC |
| 516. | PITX2 | AGCTGGGCCAGGGCCGGGACAAAGGTTTCCCAGGGAGGGCCAACTCTTCCGTGTCTCTGGCGGG |
| | | TTTTCCTTGTTAAAGGCTCACAGGTTGGAGCCTGTTCGCGGCTCTTGGCCTGGTAGGGATTTTATTAGCTCTGCTCTGGCAA |
| | | CTGCAAGCCAGGAACACAATGTCCTGTGCAGGGGATTGCCCATGCAGCCCAGCTCGTGAGATCGCGGGATGGCGGGGCAGTG |
| | | AGCCGGTGCCGCTCTGGGAGCCTGAGCCAGGGCGGCAGTCCTGTCGGCCTCGGAGAGGGAACTGTAATCTCGCAACCAGGCC |
| | | GCCGCGAGGCCTTCTGCCTTTGCAAAGCTGCGCCCCACCGGCGCCCTCCCAGGCGGCGCTGCCTTCCACATTCTCTCCTGGT |
| | | CTACTTGGCCTGTACCTCCACAACATCCTCCCCCCATCCCTCCCAGACTCCGTGCTGGCTCCTACCCGGACTCGGGCTTCCG |
| | | TAAGGTTGGTCCACACAGCGATTTCTTCGCGTGTGGACATGTCCGGGTAGCGGTTCCTCTGGAAAGTGGCCTCCAGCTCCTG |
| | | GAGCTGCTGGCTGGTAAAGTGAGTCCGCTGCCGCCTTTGCCGCTTCTTCTTAGACGGGTCCTCGGCGCCCACGTCCTCATTC |
| | | TTCCCCTGCTGGCTTTTATCTTTCTCTGAAAACGAAACACACACACTTTCCCGTCAGCATGCCCACCTGCAACGCGGACGCC |
| | | AACTGGACCGGCGGCAGAAGCCGTGGAAGAGCTGGGCTGCCTGGCGCCGGAGGAGGGTGCGCGCGGCGGCTCCGGGCCGCGA |
| | | GGAGCGCTGCGCCTGTGGGGTGTGCAGGCGCAAGTGTGGGTGTCCGCGCCCCATTTCCTCCCCTCCCCCAGCGCCGCACGTT |
| | | TTATTTACATGTTTATCTCACTGCAGCGGCACATTCACTTTTATAGCCTGTGCTTTCAAGTATATTTATACACCTCTGCGCA |
| | | GACACACCAAATCTCCTGGGACGCGCACACGCGCGTGGTTTACAGACCCCCCTCCCCCTCGCAGAAAGCTCAGATTTCCATG |
| | | CGGTTTGGGAAGGCTAGGAAAAGATGTGGGGATTCGGTTGGGCACCGAAGTTCGCCGGCCCTTTCCCAAAAAAAAAAAAAAA |
| | | ATGCCTCTTCGCGAAGGGCATTTCTGAGTGGTTTCAGGCAATTTCCTAACGAGTGGAGCTCCTCGGGAGCTGAAAGCCGAGA |
| | | GGAAAACAGGGACAGAGGTCGGCGGCCTCTGAAGGTCCTCGAATCAAGATGCTGGGATTTTTGTGACCCAGGAAACAGAAGG |
| | | GAGGCCAGGGTACGAATAGAGAGGGCGGCAGAATTGCTCGCGCCCTTAGCGCCCCAGGAGCCGGGCCGGTCGAGGGAGAACT |
| | | AAAGGGATGCGGGGTAGTCAAAATTCCGGCTCCCGGAAGTTCTGCGGGGAGCCAGGCGAACGACCACTCCCACCACGCCTCC |
| | | CCCCGGAGGGGCTGACTTCCTTGGGGCGAGAGGGAGCGGGTGGCGCAGAGCAGCTGAGCGGGAATGTCTGCAGGGCGGCGCG |
| | | GCGCCTTACCTGCGGCCTCCGGGCTGGAGGTGTCGGAGATGGTGTGCACCTCCAGCCTGTGCTTGGAGGAGTCCAGCGACCG |
| | | GGGCTGACCGGGAGCCAGAACCGAAGCCATGGCTAACGGCTGGGGATGGTGACAGGAAGATGAGGAGACGGCCGACAGCTTG |
| | | GTCCCCGCTGCTCGGTGCTCCAAGTGAAGCGGGCCTTTCATGCAGTTCATGGACGAGGGAGCGCGACGCTCTACTAGTCCTT |
| | | GGCTACTGCCCCGCCGAGCCCCCGTAGCCGCCGCTGCCCGCTCCGGGTCGCGCTCTAGGCGCGGAGTTTCCCCGCTGCGGGG |
| | | AGAGCCAGGGGACGCAACCCCCGCCGAGTTCTCAAGCCAAGCTGCCCCCGTCTCCTCCGGAAGGCTCAAGCGAAAAAGTCCG |
| | | GAGACGGAAAGTCAGCGGGCAAACGAAGACATGGGATGTGGGCAGAAGGGCACCACTCAGAGCGTCTTTAGGGAGCAGGCTT |
| | | CCAAGCTCCAAAGCGAAACAAGAGTGGGCAAAGACCCCCTTCTTCTCTCCCTCCCTCCCCCAAGAACCCCTCCAATAAGGAA |
| | | AGCTAACGCCGACCGCGCTCTGCCCGCCCCCCCCCCACGCGGCAGCCCTGACAGAGAAGTGTCAAGAGTGACAGGGACAGGT |
| | | AGGTGATATTAGATCCCCTGCGGCGGCAGCAGCCGCTGCAGCCACGACGCGGCCCTCTGAGCGCACCCTCCGCAACGCGCAC |
| | | ACGCACACCCCTCGGGCGGTCGAACAGGAGCCGGGCCTTGCCGCAGCTCAGCTCCAGGCACCCAGGCGAGCGACGGACCAGA |
| | | TCTGCGGCTCCGCGCTTCCCTGTTGGCCTAACATCTTAAAACCAGAGGCGGGCTTCCTGGTGCCGAGACGTCACTCCGCCGC |
| | | GGCCCTCCCCAGCCCTCTCCGCCTCCGCCTCCTCCCAGACCCTTCTCCGGGTGCGACTGACGTGGCTCCGCACCAATCAGGA |
| | | CGCCCCGAGCCGCGGTGGAGGGACTGTCCTGCCTGCACCTATCAGCAGTGCGGGGCCGGGCTACTGCCTCGCCGTGCGCACT |
| | | GGGTCTACACAGGCAAGCTCCCGGGAATTCAGCTCCTGCCCAGCCCAAGGCGATCCGGCTTTTAGTACGAACCCAAAGGTGA |
| | | AGAGATGAGGCTAGGAGTCGAAGGCTTGGGAGAAGAGAGTGGAATGGTCAAGAAGAGAAAGGTACAAGGATCAACAAGACAC |
| | | CCACTCTTTGTGTCTCACTACATCCATTTCCA |
| 517. | PLCG1 | GTAGGGGAGATGCAGGTTTGGAGTCACCGGTGCCTGTGTTGTTGGGCAAAGCCAGGGGAAAGGT |
| | | CAGGTCGCCTTGGGATCTGGCCTAGCACTGAGTGCAGAGGAAGAGAAGCTTCTGAGATCGAGAGGCAGCGGCTACAGGAGGA |
| | | GGAAGAAAGCAAGGCGGGGAAAACCAAAGTTCAGGAGAGCATTTGCAGGAGGGCGCTGCGGGGTAGGGTCTGAGTCGGAGCC |
| | | CCGAATCGGACTCGAGTCCTGTCGCGTGGGGACGGAGCGTGCAGAGGCCCATGCGAGGGACAGACACAAAGGCCCTGGGAGC |
| | | TGCAGGTCAGACCACTGGACAGCGCGGCCGCGGACCTAACGCCCCTGTAAGGGGTGCTCACGCTTGGGGGGAACTCTCCGAA |
| | | AGAGAGGACCACTGAAAGCCACCCTGGCAGCAGGGGCGGAACAGACAGACGTGGGTCTGGCCGTCGCCAGAACTGCCTGCGG |
| | | ACCAGCCCCGCGCTGCCGGGGGGGCGGGACATGTCGGCTGTCCATCAGTGCCCGCGACCAATCCGCAGGCAGCCCCGCCCCC |
| | | GCCCCTCCCGGGAGAGGGCGCGCGGAGGACCCGCCGCCGCCGGTGTGAGGCGGCCCGTCCTGGCTCCCTTGTCCGGGAAGCC |
| | | CGCCCAGGTAACTGAGTTTGGCCGGGCATTCCCGGAGGACGCGCCATCCCCTCACGTCCCGTCCCGGTCGCTGCATGGGCGG |
| | | TGTTCGGGACGCGGGGGCTGCGTCTGTGCGGGACCGCGGGGTAGCGGCCGCCCGTGCGAGTACGCCTGACTGACGCGCCGGC |
| | | CGCCGGGCCTGCGGCCTGTGGGCGGGGCTGCCGTGCGTGACAGGGCCGCTCGTGGCCGCCGGGCTGTGTCCGGGGCCGCGTG |
| | | AGAAAGCTCTGCGGGACTGAGGGCTGGGTGGGTCGACCGGGAACGGCGCGCGCTCCCGCCGCCATCGCGCCTCCGTCCCCGC |
| | | CTGCGGCCTGTCTGGGGGTCGCGGGGCGCAGGCGCGGCGGGCCGACGGGCGGGGGTCCTCCCCACGGGTGCGCGGGCGCCTT |
| | | TGTTCCCGGCGCCGAAGCGGGGTGGGGCCTCAGGGCAGCCCCGCCCCGCCGCGCTGAGGCCCCGCCCCGTGTCCGCCTGCAG |
| | | GAGCCGCCGCCGGGTCCCGCTCGTCTGCCGCCTCAGCCTCAGCCCCAACCTCAGCCGCCGCCGTTGCGCTTGCTCCCGGGCG |
| | | GTCCTGGCCTGTGCCGCCGCCGCCCCCAGCGTCGGAGCCATGGCGGGCGCCGCGTCCCCTTGCGCCAACGGCTGCGGGCCCG |
| | | GCGCGCCCTCGGACGCCGAGGTGCTGCACCTCTGCCGCAGCCTCGAGGTGGGCACCGTCATGACTTTGTTCTACTCCAAGAA |
| | | GTCGCAGCGACCCGAGCGGAAGACCTTCCAGGTCAAGCTGGAGACGCGCCAGATCACGTGGAGCCGGGGCGCCGACAAGATC |
| | | GAGGGGGCCAGTAAGTGCGCCCACTTCCTGCCTGGGCCCGCCCCGCGCGGGGGTCGTGGGAGCCCGGCCCGACTGCTTGCAC |
| | | CCCGGCCGGCCGCCCCAGCGACTTGGGCAAACTTTCGGGCCCTCCCAGACTCCCTCCGGGCCCCGCCCCCGCTTCGTCTCGG |
| | | GTGGTCACTGGGGGCGGGGGGCATCCGGGTCCTCGGTCACCTGACAGGACACCCCCCCTCCCCCAGCTGGGGGGAGTGTTCC |
| | | AGGCGCTTTGCCCTGAGGCCTAAAAATCCTCGCGGGCTGGAGACCTGCGGTGCAGGCATCGGGCCCCCCAGACCCTGGGAGT |
| | | GGTGGCGGCGTCGGCGAGGGGAGCTAAGGCAGTGGCCCCCACCCTGCACGGGAACCTGGGGCCTGACCAGACGGTCCCCGCC |
| | | CACTCTTTATCCAGAAAGAGCAGTCTGTGAAACTCTCCGGGCCCCCAGGCTGGGCTCTTATTTGCAAAGGAATCTTTGGGTT |
| | | CCCTAAGTAGAACTTAGGCAGATGTTGGGTAGGGCTGGTCTTGGAGCAGAGC |
| 518. | PLEKHC1 | CTCGAATCATTACATATGAAAATTCAGAGAATAGCAAAACAAAATAAGACGCAAAGGGGAACAG |
| | | GTCTATCTGCTTAAATACTGAAAGATTTTACATCTCCAAAAAGCATTTGCGAGATGCCAAGATTTCTCAAATGGGCCCCTCC |
| | | CCCGTCGTGAGCCTCTTTCCTCCCCCTACCAATTCTGGGAAGTCATTAGTTGGACGCGGCTGGGATGCTACTCACCGAGTTT |
| | | CTCCACCAGCTTAAGCATCACGCCTCCAATGTGCACCTCGCCGGTCACTCTCAGGGTGACATCGCGGTTCAGGTCCGTCACA |
| | | TGGACACTCAGTTCCCACGTCCCGTCCGCGTAGCAGCCATCTGGCATCCTTATCCCGTCCAGAGCCATGGCTCCTTCCTGCG |
| | | AGCGCGGAGGAAATGGCTCTCGTAAGCGTCACTCCCCCAAAGAAAGGCGAATCCCACCGAATTCGCAGCGCCGGCCACGGGC |
| | | TGGGAGGTGGTGGAGGACCCGGGGCTTTCGGCAGAAACTCGGGAGGGCGGCGGCGGCCGGGGCTGCGGGAGGACCCTACGCC |
| | | CCGCTCGCCCCGCAGCGTTCCTCGGTCCCGGCGCCGCCCGCCCCACACCGTCCCCGCCTAGCGGACCGCGGAGGGCTTCACC |
| | | TGCCGGCCGGCCCCGAGACACAGCGCGGGCTGACTCCCCGTTCCCCTCCCCGCCGCGCCCCCTCGGGTCCCGGCGGGGTCCC |
| | | GCTCCCTCACCGCGCGCTCCTAGCGCTCCGGGCCCGGGACTCGCGCGGCAACAGGCGAGGGGCTGGAGGCTCGCGGGGCGGC |
| | | GGTGTCCGCGTCCGGTTCCTCGGCTGGCGAAGCGCTGCCTGTGGCCGGAGCGGCTAATGGAGTCCCGTCGTCGCGGCCCCTC |
| | | GCCTGGCTCCCCGCGCTCCACCCTCTCCCCGCCCCCTCTGTCCGAGCCTGGCTGGGCTGGGCGCGCTGGCTCCCTCCTTCGC |
| | | CGCCCGCAGAGCTGCCCTGAGCGGGTCCGGCCGGCCTCGCCTCTGCCCCCGCCCTCCCGGCGCCGCGCTCGGGGGAGGGGGC |
| | | TGCGGGTCCCGGGCGGGGCGGGGCGGCGTGGGGCGGGGCGGCGCGGGGGTGGCGGGGGGCGCGGCGGGGCGGCAATCTCGGC |
| | | CCCGCGGAGCGCTGCCCTTTTATGGAAATGAAGGACCCGGCTCAGGAATTGAATCCAACATCCGGGCTCTAGGCGGCGGGCC |
| | | CTGAGGAGGGGGAGCGGGGGAGCGCGGGCGGCCTTTCCCGGGGCGCTGGTGGCCGGCGGGGCGTCTAGAGCTCAGGCTGGAC |
| | | TGGGGCCCCCGCGGCGTGTGTCCGCGCTGGCAGCAGGGAGAGGGGGCTTGTCCGTGAGAAACGGCCCCAGGAAATTCCCAGG |
| | | GCAAGGTCGTTTTCGGAGAACAAAGTCCTCTCGCCCCCTCCCCACGCCTGCAGCGAGACAAAAAGCCTCAGGGAGGTGAGAG |
| | | GTGCCGCACCCCGGGAGCTGGAGAGAGGAGTCTGGGACGGAGAAACTTTAAAAAGTAAATGCCCAAACCAACTCCTTGAATG |
| | | TCCTACAAAAGAATAAGAAAACCCCGAGGGAGGTGAACCACAAAAATTGCTTTGCAAGCAATGTCCGGCACCATCAGCAACT |
| | | CCTACTGGA |
| 519. | PMP22 | TGAGCCGAGATCGCGCCACTGCACTCCAGCCTGGGCGACAGAGCGGGACTCCCTGCCCAAAAAA |
| | | TAAAACAGAGGAGACGAGGTCTCTCTCTGTAGCCCAGGCTGATCTAGAACAGGATCCTGGGCTCCAGCGATCCGCCCGCCTC |
| | | GGCCTCCCCCAGCGCTAGGCCCCCAGGCAGGGTCCCCGGCCCCGCCCCTCCCCGCACCCTCCCCAGCCGCGCGTCCACCCCG |
| | | CAGGGCCTGGCCCGGGTCCCGCCGCTCTCATCGCCCCCGCCGCGCGGCAGGTCCCAAACGCAGCTGCGCGGAAGGCGAGGCC |
| | | CGAAAAACCTCAGAAAGAGCCGAGCAGGGAAAGGCCGCGCGGCCCGCCTCGTTTCCCCGCAAAGAAGCCGCGCCCGTAGCGC |
| | | CTCCGCCCGCCCCGAGCCCCTCCGTCGGCGGTCGGGGCGCGCCGCCCTACCCCAGTCAGGCGCGGCGAGTGCGGGCGGCTGC |
| | | GGGAACCGGGCCTCCCTCCCGCCTCGGCGGCGCCAGCCGAGGACGGCCCCATGGCACCCTCCGGAGGGCCGGCCCGAGAGCC |
| | | TCAGGAGGGCGCCCCTCACCTGCTGGCCTCGCCATCCGCGCCTGGGTCCGCGCGCCGCCGCCCGCCGCTCCTCAGAGACATG |
| | | GAGCCGTTGGCTACCACCTCTGCTTCAGGGGAGAAATTTGGCGCGCTCCCACCCAGACTCGCGAGAGCCGGAAATGCCGCCG |
| | | TCCATCAAGAGGCGCGCTCCGCCCCTCCCCCGCCCCCCGAGGCGCTCCAATTGGCGCCGGCGCCCGGCGGGGGAGGTGGCCG |
| | | GCAGGGACCGGGGAAAAGTGTGTGGTAGGCGCGCGCGGCGCACCGTGGGGTTGGGACCGGGCGGCGCTGCGGGCCGCGGTGG |
| | | GGCCGGAAATGTCAGGCGGTCCCCACTCCGCTCTCGGCGCCTCGGGCTCCGCGCCCGGCCAGCCTGAGGTGGGGTCGGTGCC |
| | | CCCGGCGGCACGGCGCTGGGGAGGCGATGGCGCCGGCCGCGTCCAGGCTGCGGGCCGAAGCCGGGCTCGGGGCGCTGCCGCG |
| | | GCGGGCGCTCGCCCAGTACCTGCTCTTCCTGCGGCTCTACCCGGTGCTCACCAAGGCGGCCACCAGGTGAGCGGGGGCGCGG |
| | | GAATCGGACGCCGCCCCGGCCCCAGGTCGGCCGCAGCGGCGCGGGGCCTGGACGGGAGCGCCGGGCCGGGACCAGGCTGGGG |
| | | GCGCGCCCGGGTCAGAACCCCCGGAGCGGGCGCCCTCCGACCCGGCGCTGAACTTCCCGCGGCGACACCCGCGTCCGCAGTC |
| | | AGCGATTGTGAGGCGCCTTGCGGATCCCGCCCTCCCAGCGCGTGGAGGGCCCGCCCAGACCCGGGGGTCGGCGCTGAGGTTC |
| | | CACCCAAGGACCCAACAGGGGCCCCGCGCGCCCGGGGAAGCCACGGCAAGGGGAGCGGGTCCGCAGGCGCGGGTGAAGACAG |
| | | GGCGAGGGGAGCGGGTCTGCGGGGCGCGGGTGAAGACAGGGCGAGGGGAGCGGGTCTGCGGGGCGCGGGTGAAGACAGGGCG |
| | | AGGGGAGCGGGTCTTGGGGGCGCGGGTGAAGACAGGGCGAGGGGAGCGGGTCTGCGGGGCGCGGGTGAAGACAGGGCCAGGG |
| | | GAGCGGGTCTGCGGGGCACGGGTGA |
| 520. | PRAME | CCTGCCCTTGGCTGGGTAATCTCTGGATTTACCTACTGTCTGTAACCCCCCGGGCTGAAGAGAC |
| | | CACCCCCCGGGAGTCAATTAACTTACTCTCTGGAGCGCGCGTTCCTCCCTGCTCCCAGGAGTCGGTTTCCCAAAACTTTCTG |
| | | AGGCCCGCGCATGCTCCCCTCGACTCCCCGTGTTTCCACTCTCCACAGAAATCCACGCATTCACGCCCCTCCCCTCCCCCGA |
| | | GCCTGCAGAGGACTCCGCCCTGCTTTCCCTACATTCAGGGCTGCTCCTTTTGTCGCCAATACAAACCTGTTGACAGGTCACG |
| | | CCTGGGAAGCGGGTGGGGTGTCCCGGAGCGGTGCTGAGGCGCTGCAGGCCCGGCTTCTGGCTGCGGGGGAGCTGTACCCTGA |
| | | AGCCTCGCCGGAACTCGCGTCTGGGGCCAGCAGGGGGCACTA |
| 521. | PRG2 | AACTCAACCAGGTGCAGAGGCAGGGAGATTTCATACAGAAGACACAAACTCCCGCTGCCAAGTT |
| | | GGTGTTATCTTCAGTTTACTGACAATGAAACAAAAGCTCCCATGGATTTCAGGAACTTGCCCAAGGTCACAGGGCTAGTTTA |
| | | GTCACGACGCAGGCCATTCTACTGCCAGAAATACCCCCAACTCCCATGACCCTCGCCTAGGACTCGCAAACCTGGTCCCCGC |
| | | CGCCCTTCCTCGCATCAACTTCTACCAGGAAAGCCTCCGGGGGCCGCTCCCCGCCAGCCTCCGCACCCCGCTCCAGCCTGCG |
| | | GCCTGCCCTCCCCGCAGAGGAGCCCGAGGGGCCAGGCCGCGCTCGGCGCCCCATGGCGCCCGAAAGGGGACCCTTCGCCCTA |
| | | CCCGCCTGCTCCGCGCCGGGGCTCTCCGCGCCCTTTCCGCACGGGCCAGGTTCGCATTCGCGCCTCTCGCAGCCCCTCCCAG |
| | | TCCCCTGCTCGCCTCCGCCCCCTCCTGCCCGCCCGGAAGGGGCTGGGGCAGACCTCCCACTCTCCATCACTTCCTTCTTCTT |
| | | TTCCCTTGCTCACAGCCTCCCGCGCCCTTTTTACCTCTCCCTCTTGAAACTTCTCCCTCTAGAACCCCCTAGAACCCCAGCG |
| | | GTGTCTTTCCCTCCCTCCTCGCTGCCTTTCAGCCTCCCAGCCCCCTTGCCTCTGCCTCCCCTAACCAAGTT |
| 522. | PRO2730 | TGACAGCCTGAGGAGCTGAAAACGCTACCTGGTGGGCCCAGGGTGGGCGGACCGGGCGTTTAAA |
| | | GGTCAAGGTCAAGGTGGGGTTTGGCAAGTGATGCATAGCGCTCAAAGGAGACCGAAGACATACAAATAATATTATAAAATTA |
| | | TGACTATCGTTATCTTTTTTTTTTAAGAAGAAAAAAATAAAAGGTCTCCTGGACGGCTGCGGGGTGGAGGCGGCCCTGGCAG |
| | | GAACGCGTGCAAAATAGGCTGGGGGCTGAAGTCGGGACGGAGGGCAGGCCGAGGGACCTGTGCTGGGCCACCGGAGGGAGGC |
| | | ACTGAGACTCGGGCCCAGGGCCTCCGCCGCACTCACTTGCCCTCCTGGCAGTCATAGAGCACGATGGAGTCGTCGTCGCTAC |
| | | TCGAGATGACCGTCTCGCCGTTGGGGCTGAAATCGAAGCAGTTAATCTTGTCCGAGTTTTCGCGGAACACCTTAGCGACGCG |
| | | GAAGCTCCGCAACACGCTGTCGGTCAGCTTCATGGCGGCGGCTGGGGAAGGCAGCGGCGGCGCAGGGCCGGGGCGGGGCCCG |
| | | GCGGCGAGCGGGCGGGCTGCCGAGGGGCCAACCCAGGCGGGGCGGGCGCCGCGCCGGCGGCTAGCGGGAAGTCGGCCAACAG |
| | | TTGGGCCGCCTCCTCCTCTTCTTCCTGCTTGGTCGAGGGTCTTCTGCCTGGACTGTGGAGCCTCGCCGACCGTTCGTCCTCA |
| | | CAGCCACCTCACGGACAACCGGCGCGTCGCCGGCTCATTGTGTCCGCCATTTTGGGGCGACAGGCAGAAGCTGAGGGCGAGG |
| | | AGCCTCAGCCGCGGCGCACGCCGGGAAAGGGGGCGGGGCGGGAACGGCGGAGCAAGGCGGGGAGGCGGGGTGGTGACGTCAG |
| | | CGCGGCCGCGGCGGAGTGTAGTGGCGGGGCGGGCTGCTGGCAGGGAGCGGCCGAGACGTGCAGGGCCTTCCCGGGGGATCGG |
| | | AAGGTTGCGGATGCCTTGGGAGCCGGGGGCCCTTGCGCGGGATTGCAACCCCCGCGAAGACTCTGGCGCCTCCTGCAGGCGT |
| | | CCCGATCGCAGGCTGCGTGGGAGGGGGTACTGCAGGGTCGGGTATGCTGCTGGAGGCCGGTTTTGGGGGTGACTCAGGTGAG |
| | | CCGCTCGCAGGCAAGCTTGACCGCTAGTCCACCGCAGCGTCCGCTTAGACAGCTTCGACCTCTTTACTTTTTTTTTTTTTTT |
| | | TGGAGATGGAGTCTCTCGCTCAGGCGCGCGCCACCACGCCCAGCTAATTTTGTATTTTTGGTAGAGACGGGGGTTCACCATG |
| | | TTGGCCAGGCTGGTCTCGAACTCCTGACCTCACCCGCACCCGGCCTCGTTTTCTTAATCGAGGTAGACTTTAGGTACAGTAA |
| | | AAATCAGCCTT |
| 523. | PSCB5 | GTGCACCGAGGAGCCGGGCAGCCACCGCCACGATCCAGCTTTAGGACATTTCCATCACCCCACT |
| | | CAGCCTTCCTGCGCCCCTGGTTCCTCATCTATAAATAGAGATAATATACGCCTAACCCTCTAGGTCACCAGCACATTGCACC |
| | | CATAATTATTATACTTGTTGATGCCACACTTTCAGGTTTTAACCTTTCCAAAGCGCAGCCCCGCCCCAGCGCAGCCCAAGTC |
| | | CCTGCACGCAGAGGCGCCGATCACGGGTGGGAGGTGGATCCCGGAGGCCCCGCCCCAGGCCCCGCCCCTCCGTGCGCCCAAG |
| | | CTCGACCGCCTGAAATCGGGGAATCGGGCCTCTGCCGGCGCCCTGGCAGCGGCGCGGGGCGTGGCTCCGGGCTGGATTGGTG |
| | | GCGCCTGGGCCCCGCGAGCGCCTGCGCAGTGGTCAAGGCCGCGCTCGCGCCGAGGGGCTGCGAGAGTGACCGCGGCTGCTCC |
| | | AGCGCTGACGCCGAGCCATGGCGGACGAGGAGCTTGAGGCGCTGAGGAGACAGAGGCTGGCCGAGCTGCAGGCCAAACACGG |
| | | GGTGAGCGCATCAGCCCCCGCCAGGCTTGGCCCTCGCGGGGCGCCGCCCTCGCCCGGAGTGTAGGGCGCGCCTCCGGGGCGG |
| | | AGGCTCTGGGCGCCTCCCCGGGGCCCCGGTCCCCTGCGCTGCCCTGGCGGCCTCGTGGCCGGCTCAGAGGTGGCCTCGTCGC |
| | | TTTCCTCGTCGCGAGGGGCCCAGGAGGCTGGCGGCGGGGGCCGCCACGTGCGGGACCTGCCCTGGCGTTGGGGTGTGGGGTC |
| | | CCCTAGCCTGGAGCGGGCTCGTCCTGGCGGGCCTGGATCCAAGCACAATCTCAGCTTTTGGAGCCAGCAGCCGGACCGAATT |
| | | CAGTTTTTCCGGAGTTCAGTTGTTATGACCCAAGTTTAGCCGACAGGTATTGAGGGTCCGCTGTGTTCCCGGTGCTGAGAGC |
| | | GCGGCAGGGTCGGAGGAACGCTTGCCCCGCCGGGATGACAGGGAGGGGCCGCCTGGGAAGCTTGGATGGATCACACGCCCGG |
| | | AGAGGCGTTTCTGATAAGGGACCGGGACCTTAGGGACGGAGGGCGCAGTCTTTGAGTTATCGTTGTCCCTAATACCGAGGCC |
| | | CGTTTTATTTTCCAGAAATTATTCCTACATGTGAAACACGGTTTCGTAATACAGGGATTCCCCTCCCCAGGGTGCTCTGTAT |
| | | TCCAAAGCCTTCTGAGGTTGTGGTTTTAGACGTTCATCAAAACGTTGTAAGTGGGAACTAGTTCATTGATTTCTGTGAGGCC |
| | | CAGTTTCTTATTTGGGGAGTTGTTTCTACCACCGCAAG |
| 524. | PVALB | TGTTCGATTTCCCCCCAAAACCTATCCACTCGTCGTGCCTCCTGGCCCCTCAGGAATCAGGACC |
| | | CTCTCGGGCACACCAGGTTCAGTCCTGCAACCTTCGTTCCTAGCCCGAGCTCGCCCCACCTTCCAACCAGGCCCCGAATGGC |
| | | CCCCCATCCCACCTCCAACCCAGGACTCCCGGAGGCCCTCCCTCTCTCCAAGCCGGGGAGCCCGGTGTCGACCCTCACGCCC |
| | | ACCCCCGCAATAACTGGGCGCCCCGTCCCGCGCGGTCACCTGCATCCCGCCGCCACCGTCGGCCGCCGCCTCCTCAGTGCGC |
| | | GGCCGCTTGCTCAGGCCGGGATCGGCTCCGTCTCCTCCCGCTCCGGGCACCGGCAGCTCCAGCTCCGACTCCCGCTTCATGC |
| | | CCCGGGCGGCGGCGCCGGGCCCGAGCTGAGGCGGCTGATGCGGCTGGGCGCCCTCCGCCATCCGCCCGCGCCCCCCTCGCCT |
| | | CCGGGAGCCGGGCGACCCGCGACAGGCCACCTCCCCCTGGGCCTCGGCCCCGACTGTCGCGACAGGCGGGCGCGCGCCTGCC |
| | | CCCGCCCCCGCGTGCGTGCGTGCGTGCGCGCGAGCGGACTCCGCGCCCCTCCGCGCCTCGCGGCCGCCGCTCCTTGCCTGAC |
| | | CGCTTGCTCCCCGCCCGCCCGCCCGCCGGGTTGTCGGCGCGGGGCCACTGGCGGGTCGTGATGAGCACTCGCTCGCGCCCCC |
| | | GCACGCACACGCGAAACCCGGCCCGGCCCGCCGCGCCGCCCCGCCTCTCGCACTCCCGGAGCTCGCCCACCGGCCGCGCTGG |
| | | CTCACACTCTCCCTCACAGCACGCCGGCCGAGGGAGGAAGGGGGCGGTCCGGGCTCCCGAGGCGTGGGGAGGGCTGTTTATT |
| | | TTGGGGGGAGGAGGGGCGCGAGGCAGGAACGAGCTGACTGGCCGGGATCCTCCGACCCGCCACTGTGGCAGCACCGGGAAGG |
| | | CGGGGAGAGAGAAAGAGGGAGGGAGGGAGGGACCGGGATGTAGAACTCCAGCCCGCGCGGGAGGCTACGGCGAGGGGGGCGG |
| | | TGGCGGCCCGCGGGGGGGGCGGGGCCAGGCCCCCTCGGCAATCTCCGTAGTCTCCTCGCTGGCTGCCCGAGGGAGGCCGGGA |
| | | AGCGATCGGGGAAGCTCGGGAATCTCCGGCACGGGCCTGGGATTGTCCTGGAGGCACAGCGCGGCTGGAGTGCGGGGCAGCG |
| | | CGGGGGGGGCGGGGTCTGTCTCCTTTCTGGGCGGGGCCGTATCCTGGAGCAGGCGGGGCTTGAGAGACCCGAAGGCCAGGGA |
| | | GTGGCTCCTGCTTGCGGTACTAGTTGTACAGAGTTAAGTCCTGAGTTTACTCGCCTGAGCACCTTGGTTCCCGGAGAGGGAA |
| | | TGGGCACTCTGTGAGAGGCAGGCTATTTGCCTGCTTCCCCTCCCGCAGAAGAAAAAATGTCTCAAATTGGAAGGTCGAGGAA |
| | | TGAAGCCACCCCTCTATGGTTCACC |
| 525. | RARB | AAGAAAACGCCGGCTTGTGCGCTCGCTGCCTGCCTCTCTGGCTGTCTGCTTTTGCAGGGCTGCT |
| | | GGGAGTTTTTAAGCTCTGTGAGAATCCTGGGAGTTGGTGATGTCAGACTAGTTGGGTCATTTGAAGGTTAGCAGCCCGGGTA |
| | | GGGTTCACCGAAAGTTCACTCGCATATATTAGGCAATTCAATCTTTCATTCTGTGTGACAGAAGTAGTAGGAAGTGAGCTGT |
| | | TCAGAGGCAGGAGGGTCTATTCTTTGCCAAAGGGGGGACCAGAATTCCCCCATGCGAGCTGTTTGAGGACTGGGATGCCGAG |
| | | AACGCGAGCGATCCGAGCAGGGTTTGTCTGGGCACCGTCGGGGTAGGATCCGGAACGCATTCGGAAGGCTTTTTGCAAGCAT |
| | | TTACTTGGAGGAGAACTTGGGATCTTTCTGGGAACCCCCCGCCCCGGCTGGATTGGCCGAGCAAGCCTGGAAAATGCAATTG |
| | | AAACACAGAGCACCAGCTCTGAGGAACTCGTCCCAAGCCCCCCATCTCCACTTCCTCCCCCTCGAGTGTACAAACCCTGCTT |
| | | CGTCTGCCAGGACAAATCATCAGGGTACCACTATGGGGTCAGCGCCTGTGAGGGATGTAAGGGCTTTTTCCGCAGAAGTATT |
| | | CAGAAGAATAAAGGTGTGTGGGAGCTTTTACTACACCTTTATCTTCAGTAAGAGACAGAGAACACAGTTCCATTTTTAATGT |
| | | TTAAATTTCATTTCAAAAAGCAGGTCTGTAGTTTGTAACCATGACAATTAAAATCTGTGCTAATGCACGGCAGTCTATAACA |
| | | ATTCTACAAGCCAATCAGACAGTACGTGACATTTCAATGAGTAA |
| 526. | RASSF1 | GTTGTTTTTTGGTTGTTTTTTTCGTTTTCGTAGGCGCGCGGGGTTATTATTTACGCGCGTATTG |
| | | TAGGTTTTTGCGTACGACGTTTTAGATGAAGTCGTTATAGAGGTCGTATTACGTGTGCGTGGCGGGTTTCGCGGGTTGGAAG |
| | | CGGTGGTTACGGTTAGGGATTAGTTGTCGTGTGGGGTTGTACGCGGTGTTTCGCGCGATGCGTAGCGCGTTGGTACGTTTTA |
| | | GTCGGGTGCGGTTTTTTTTAGCGCGTTTAGCGGGTGTTAGTTTTCGTAGTTTAATGAGTTTAGGTTTTTTCGATATGGTTCG |
| | | GTTGGGTTCGTGTTTCGTTGGTTTTGGGCGTTAGTAAGCGCGGGTCGGGCGGGGTTATAGGGCGGGTTTCGATTTTAGCGTT |
| | | TTTTTTAGGATTTAGATTGGGCGGCGGGAAGGAGTTGAGGAGAGTCGCGTAATGGAAATTTGGGTGTAGGGAT |
| 527. | RBP1 | CACAGCCTGCCTGTGCCTCCCCTGCCGCCCCCAACAAAGTTTTCTGCTCTTATGTTAACCGTTT |
| | | AAAAACAAAAAGTAGCAAACCAAGCCCCAGATTTACTAATGAAATTACCCACCTCCAGGGCCAACGAGCAGACATTTGCCAC |
| | | CGGTTAACCTAACAACCCCGAATTCTCCAATCTGCAGCCTGGGGGCTGCCCTGGGAGCGGTGGCCGAGGACGCCCTTTCTGG |
| | | CGTCCGGGGGCACCGGACCGCGGCTCGCCGGGATGGAGGCAGGCAGGCAGCGTCTCGGGAGGGCTCTAGGGAGCGGCCCGGA |
| | | GCTGCTCGGCGGGGCGCCCAGGGCGCAGCGGGCAGCGCTTACCGAGGGCGCGCAGGTACTCCTCGAAATTCTCGTTGACCAA |
| | | CATCTTCCAGTACCCAGTGAAGTCGACTGGCATTTCGGGGAGTGACTGGAGCCAGTTGGCCACAAGCGGGCGGGACGGCTGG |
| | | AGACTGCCGGGACAGCGGCTGCCGGTGCTACGCGGGTGGTGGGCGGCCCGGAAATGAGCGCCCTCCGGGGACAGGGGGCTCT |
| | | GCGGGGCGGCGACAGCTGGATTCCCAGCGCGCACAAAGCCTGCGGGAGGATCCATTGTAGCGGTCGCTCCTCCCCGCTTAGC |
| | | GAGGGCGGGCGCAGGGGCGGGGGATGTCGAAGGGTCAGGTTTGTCCAGGCCGCGCCACCTTCGTTTCAGACGTTCAGTTCGT |
| | | TTCCCCTACAAAGGAAGAGGAGACCGGAGACGGGACGTTTGCTTTGTTGGCCGAACTCTTCGGCTGCTGGGGATTTCCTTCA |
| | | AACGCCATTAACTTCTCACACTCAGCAGCCACTCGGGAGTTGGGAAACAATCTCCTGGGGTTTGTGCTGGCGTGATTAAGGA |
| | | AAGAGGCCACTTACAGGCCTTTGTCTAAAGCCTCGCGCCGGTGGTGGCTGGGGTCAGGAAAGCTGGGAGGTTCAACTACGGG |
| | | CGAGAAAATTGGG |
| 528. | RGS16 | CCTTGCTTTCTCCCTGCTCCTGGAATCTGAGCACGTGGCATCAAGTGCGCTTCTACCTCTCTAC |
| | | TCAAGCCTCTAGCGCCCCATCCCCGCGCCCACCCAGGTCCCCAGGCCTGCCGCTCCACTCCCTAAGAGTTGGTGGGACTTCC |
| | | CCCAAGCAGTTGGACAACCTCCCCTTACCTCTCCAGGCAGGTGGTGGGGAAGGCGGCCAGGGTGCGGCACATGGCTGCGGGC |
| | | GCAGGGCAGCACGTAGTAGGCAGGATGGTGGCAGGCTCCAGGAGGCTCCGCGTGCGCCAGGAAGCAAAGGCGCGGTAGCAGG |
| | | TGCTAGTCAACTGCGGTTGGGTTTAGCAGCCTGCAAGCGCCCAGACTGAGCGGCCGCTGCTTTAAATCCCCTCGGAGGAGCG |
| | | GGGCGGGGCTGGGCACTGCTGACCAATCGGAGGGTGGGGCTGGGCGCTGAGAAGGCGGAGGCGGGGCCCGCAGCCAGACTGG |
| | | GCCGGGTGCAACTTTCGCGGCAGCTGGAGCACGCGGAGCTGCACCCTCCGCCCAGAGGACGCCGCGTGTGGAAGGAAAAGCG |
| | | GCAAGAGAATTGGCACGGGGATGAGACGTGCACACCCAGTTTCCTAGGGAAAAGTACCTGCCGACCTCTAGTAACAGCAGGA |
| | | AAGTCACGCAGCGGTGTCGTGTTGAGAGAGTTTGCACGGGGGTGGTGGGTCTGGGAGGCACACAGGGCTTTCTTTGATCGCA |
| | | GGCAAGTCTGAGCCTGCTGGGTCCTGAGGACAGGCGCACAGGGTGCAAACCTGGGCAAAACAGC |
| 529. | RIS1 | ACAGGATTTGGTCATTTTCTGCCATGAATCTAGGGCACGTGGGTGCGGGGAAGGAGTCGGGCAG |
| | | GGGGATGCAATAGAGGGGAAAGGGCCCCATTTCCCTCCTCTCCGTCTTCGGAGCTGCGATCCCACCCTCAGTCCAAGGGCTT |
| | | AAACATCTGCTTTTCGGAACTGGAAGCGCAGCACAAACCTTGCTTTTCAAAGGCGCTGGGGTCTCTCGGCGGCCCCATCTCG |
| | | GGAAGAGGAACTAACGATAACTACAGCACGAAGCACACCGGCCGAGTCTCCGGCGGGAAAGGCACCCGCGCGCGCGGACACA |
| | | GGACGGACACAGGGAGGAGCGGAGCGGGTCACTTGGTCGCCACCCCCGAAGTGACGGCCGCGGCGGCGGCGGCAGCGGCGGC |
| | | GGCGGCGGCGGCTGCGGTGGTCCCTGCGGGCACTGCGGCGGGGGTGGCTGCGGTGGTGCTGGCGGTGGTCCGGCGCTGTTCC |
| | | ATGCCGTTGGGCAGGAAGCCGGCGATGATGGGCACCGGCCAGATGAGGGCGGCCACGCTCCACACCACGATGACCAGGGTCA |
| | | TGAAGCAGGCCACCAGCGTGGACTCAATGGGCTTACGGTTCAGCCGCCAGCCCGGCTCGCCCTGCAGCTCCTCCAGGCTGAA |
| | | GTCTAGGCAGCAGAAATGCAGCGGCTCGCCCTGCAGCCACAGCGGCCCGGGCGGCTCGGCCGCGGGGTAGGCTGGCAGCGCG |
| | | GTGGGCGCCCCGGCGGTGGCGGCGGCGGCGCTGGGGGCGGCGGCGGGCCGGAGGCGGCCGGTGCGGCGACCGCGCACCCAGC |
| | | CGCAGCCCACGCAGAGGCGCAGGTCCTGCTGCGCGCCGCCCGCCGCCAGCCCCAGGTGCTCGATGAGCAGCGGCGGCCCGAC |
| | | GCGGTGGAAGGCGGCGGCGAAGAAAGCGCGGCCGTGCGCGTTGGTGGAGAAGAGCAGTAGGTCGCACTGGAAGCCCCGCGGG |
| | | CGGCCCCAGCGCACGAGCAGCGAGCTCAGCATCTGCCGCTGCACGCTGATGTTGCACAGGCGCCGCCGCCGCCGCCGCCTCC |
| | | TCCGGGCCCGGGCGCTCGGGGGGCCCGGGGGCCGCCGAGGCCAGCAGCCGCGGGGCGATGGGCTCGCCGCGGAGGACGCGTT |
| | | GACTGAAGCATTGGAGGGCACCCCGAGGAGGCCGGGCGCGTCCGCGGCCCCGCCGCGGACGGGCGGCAGCGGGCAGGCGGCG |
| | | GCCAGGAGCGCGGCGAGCAGGGGCAGCATGGCCTGCGGCGGGCGCCTACGCGCGCGAGGCCGGCGGCGGTTGCATGGCGAGC |
| | | GGGCGACGGGCCGGCGAGCTCACGGTGGGACCGCGGGAGCCGGCGGCCGGGCGCAGTGCGGGCGCGCCGGGCGCCTGCCAAG |
| | | CCCGCAGGGGCGGCGCGGAGGCGGTGACGGCGGCTGGCTCGGCGCGGGGATCCCTCCAGACCCGGTTGCGTTTGGGGTTCCC |
| | | CGCCGCCCCCGGCGCCGGGGCCCTTGGGCTGCCGCGCTGCTCTGCGGGGCCGGGTGCTATCCCAGAGCAGGATTCCCCGGCT |
| | | CGGCTCCGCCTCCCGCGTGCCCCGCCCCGCCCCGCCCTCCGCTCGCCAGGCTCGGGGACTCAGGGGAGGGCGGGGTTCCACG |
| | | CGCCGGGGCTGGGAGAGGAATCGCCGGGGCGGGGCTGGGTGCGGGGGCGCGGCGGAGAGCGGATCGCCCGGGGTGCGTGCGG |
| | | GCGGGCGGGGAAGCCTGCGGACGCGCCCGGGCGCCCCCGCGCTCCGGGTGAATCGCGTTCTCAGAGGGAGGCTCCAGGGCCC |
| | | GGAGACCATCCTTTTGTCAGGCCTGAGGGGAGCTTCGCTGTCTCGCCTTAGTGCTACCGAACCTCCTTTGTTTTACAGAAGG |
| | | GGAAACTGAGGTCGAGAGAGGGGAAAGGGCCTCGCTAGGCCAACTCATGAGCCAGAAACAAGTCCGCGCCACTAGCTCTTTC |
| | | TGCCTTTTTAC |
| 530. | RPL22 | CCTCTCCCAGGCCTGGAGGGTTTCTGGGGAAGGAAGGAGGCCCGGCGGGCTGTCCCACTGCGAA |
| | | CGATCGGAGGGGGGAAGCCAGACTGGACGAGCCCAAAGATAAATAAAGGGGTTGGCTTTACTTTTTGAAAGTCTTGGGAAGA |
| | | AAAAAGAAATAAGGAAATTCACTATACTGTAAATTACCAGCTAGTGGGATGGAAATCGAATCTACTAATTTGGGGAAGTGGG |
| | | AAAATTCCTTGTAGCC |
| 531. | RUNX3 | CCCAGCCGCCGGCCCTTCCTGCCCTGCCCTTTTCTCACGGCAGCTGTGAGAGGTTTAGGGGAAA |
| | | ACCGAGGCGTTTTCGTTTCATCTCGCTGCCCCCTTAAAAAAATGAAAATGAAACAGTCGCCTACTCCCTGGCATAAAGAAAA |
| | | AGGTCCTCTAAATGGCTGGGGGCTGCCAGGGTTAGGGGTCCCCCAATCTCAACTCGCCATTCGGGACGCATAATATCCCCGA |
| | | GCAAACGTCTGGAGAGCAGTGCCCCGATCCCGGCCTAGCGCCGTCCGGTAAAATTTCGGAAGCCCGAGGGTGTGAGCAGGAA |
| | | GCTTTTGCGAAGCGGCGCGGGAGGAGGGGTGCTGGAGGCGGAGGGTAGGCCCTTTCACCGTTCGCACCCCACCCGCGGTGTC |
| | | CTTGCCCCTGTCCCGGGATCCTCTTCTCCGTTACCCGCAGGGCTGTATCTGAGCGATCCGGGTTAGGGGGGCGCAAAACCCC |
| | | ATCCGCCCATTTCCGCACCAACGTCTCTACGCAGGCGCCCCAAAACCCAGGTGGAGCGGGGCAACCCCGTTAAAAGTCATTC |
| | | CTGCAGGGCGCATCCAAAACGGAACGCCGAGGTCCCGGAGCCGAGCGCGCAGCCAGACTGAACCGGGTGCCCGGGTGTCGCC |
| | | GCGGCGTCTCGGGCACCTCCCATCCCCACTGCTCCCGAGGCTCTGGCTCCCGCAGCTCAGACGCCCGGAGCCCCAGGGCCGG |
| | | CGCCCTCCCGCCCCGGGTCCCGCACTCACCTTGAAGGCGACGGGCAGCGTCTTGTTGCAGCGCCAGTGCGAGGGCAGCACGG |
| | | AGCAGAGGAAGTTGGGGCTGTCGGTGCGCACGAGCTCGCCTGCGTGGTCCGCCAGCACGTCCACCATCGAGCGCACCTCGGG |
| | | CCGGGCGCGCCCTCCGGGCCCCACGGCCGCCTGCGCGCTCAGCGCGCCGCTGTTCTCGCCCATCTTGCCGCCGCCGCCGCCG |
| | | CAGGGGAAGGCCGGGGAGGGAGGTGTGAAGCGGCGGCTGGTGCTTGGGTCTACGGGAATACGCATAACAGCGGCCGTCAGGG |
| | | CGCCGGGCAGGCGGAGACGGCGCGGCTTCCCCCGGGGGCGGCCGGCGCGGGCGCCTCCTCGGCCGCCGCTGCCGCGAGAAGC |
| | | GGGAAAGCAGAAGCGGCGGGGCCCGGGCCTCAGGGCGCAGGGGGCGGCGCCCGGCCACTACTCGCCAGGGCCCGCCCGCTGC |
| | | GAGGCCTCGCTGGCCCGACGGCCGCCCGCAGCCTGCCCGGCTAGTCCCGCATCCTCGGCGCGCGGCCCCGCGTGCGGCCGCC |
| | | CCTCGTGGCTGTCCCGGCTGCCTGGGCCGCGGCGGGGCCCGCGCGGGGCTGTGCCGCTGCCGCCGCCTCCCGCCCCGAAGCT |
| | | CGCCCGCGGCCGCCCCGACTCCGCGGCCGCAGCCCCAGAACAAATCCTCCAGAATCAAGTGGCGGGGCCGCGGCCGCCCGCG |
| | | CGGGGTTAGTACCCCCGGGGCCCGCGGGGCGGGGCTGGCGGAGCGACGCGTCGCACAGCCAATCGGCGGAGCC[ ]CCCATCGCGG |
| | | GCACCTCGGTGGCGTTCGCGGGGAGGAACGGGGCCTGCCGGAGGCCGCCCAACGGGGAGGGGCGGAAGGCGCCACCCCGCGG |
| | | AGGAGGCCCCAGTGCCACAGCCCAGGGCCCCCGAGAGCTCTGGGAGCCCGGGGCAAATGCTAGAAATTTGCTTAGAACGTCC |
| | | GGGTCCCACGGAAGGCGCCCTTGCCGCCCTCTCTCGGGTCGTAGCTCCCTGACGCTGGGGCGCAACCCCTTCGCTCCTCCTC |
| | | CCCGCTGGCCGCGGCCGGGCTTCCCCAGCTCTTGCTGCTTCGGGCCTGTGACTTCTGCAACCCCGGGCTGGGGGCCGCGGGG |
| | | TCTCAGGGCCGGTGACGCCGCACTGGGAGCCGCCCCAAAGAGGTTACTCACCTCCCTCGTCCCGCACATTATTCTGACCCAA |
| | | GAGCCTCCACCCCACACGGGATTTTGCGCGTCGTCCACGCCCGGCCGGCGGCCTTTGCTGCTCCCAGCCCTGCGCGGCTTTG |
| | | GTCCCAGCCTCGGTGGCCCCTGTGCCAAACCGGGGACAGGCGGAAGGGAGTCTCCTAGGGACCCTAAGTAGCCTGGGGCCAA |
| | | CAACCCCTTTCCTCTCTGCTCTCCCCTCAAAACAAGTTTCAGGATCTTGCAGGCCTCGCGGCGTCGTTCTTCGTTGTGGCGG |
| | | CCTGTGGCTCTTTGAAAAACACGACGAGGCCTGCAAAATGCGTTTTTCTTTTTTTCCTTTACGCATGTAACCACGGTCCTGC |
| | | ATCGTGAAACGGTACGCGCGTCGGTGGCAAAAGAAAAACAGCAGTGGCTGCAAAGCTAAGGGCCCTCGCTTTCAGAGGAGAG |
| | | AATTTTCTTTCTCCATGCGGGTGGAAAGTGGCCTCTGCGGGTCCAACCCCACTTCTTCTTGGGCCCGTGCGCTCCGGCTGCG |
| | | CCGCAGGGACCGCGGACAGCTTCGCCAAGGCACTGCCTGCCCGCCCGGCTCCGGGTCCCCGCTCCCACTCCCAGCCGCGTGG |
| | | CCCAACCTCTCCTGGGCTTCACTGCAAATCACCCCTTCCTCTCCCGCCTCCTAAGTCTGTCGAGCAGACCTAGGGGCCGGCT |
| | | ACAGTTGGGAGGGCAACGGGAAAGATCAAGCCACAATCATTCCGAATTATCGCCCCAGACACCTCCCTAGACTCTGGGGAAC |
| | | GAACGCGTGCTGAGCCTCCCCGCCGCTTTGGAGACGGGGCTAGATTTTCGTTGCCTCCGGCTCTCGACAGGTGCAAAACAAT |
| | | GAATTCCAAGCCTCGGAAGCAAAGAAGCTTAGGATCCGACGGTGGCCGCAAGATCTCATCATGGATCTGACCCCTGCTCAGC |
| | | GCGCGCCATTTCGTCGTTGCCAAACGAAATCAAGCCCCGCGTGCGCTCCAGGGGCGAAGGACTCTGGACTCACCCCGACCAC |
| | | CGGGAGAGCTGGCCCCTACCCACCTCGGGACCTCACAGCACGCCCTCAGGCCGTGTCGAAAGGAAGGACGGCAAAGGTCCCT |
| | | TACTGAACCTTTTAAGAGAGCCTGCGCCTGGCAGTTGTCGATTGCGGACCCAGGCCCGCGCGCCCTCGGACGCGCTGGCACG |
| | | AGCAGCAGAACTAGAGGAAAGCGAGTGATCCAGCCTGGGCGCTCCCACCTCCGGGAACGTCTCCGAGAAGGCGCAGCGCGTC |
| | | GTGGCCAGGTAGGGCCCTGGCCGGGGGCGGGCAACACGTGCTGCCCTCGAGCAGGTTGCGGGACCATGACCCGCTGTTTCAG |
| | | GTGGTGGTAAATTCCATTTGTCGAATGGTTTCGGTTTGCACCGTGCCCTTTGCTTGTTCCTCCGCCTGATTTCTCCCTCTCC |
| | | GCTTACGATGGGTTCACAGACAAGTTTCCAGAGAATGAGGGACTCTTGTGGGCCCTGGCACCTGGCGCAGGGCCCGGCACGG |
| | | CTCCGGCTCTCCGTAGGGCGCTGGCTCCCCGTGGGCACCAGATCCAAGGGACCAGGGCGGCGGGGGGAGGGGGGGCGGGTGC |
| | | AGGCCCTTGGGTCCCCAGACCAAGGTCGCGGGGCCGCCTGGCAGGCACAGTGGCGGGAGCCGCCGCTAGTTGGCGCCCGCGC |
| | | CCTGCCAGCCGCGGAGGTGCGGGCCCGGCCGGGCTACAGATGCGCGCCAGCTGCGGCCCCGGGTGCAGGCGCGGCGACCGCC |
| | | CCCGAGGAGCTGCCCTTTCCTTGCCATCCATGCGGCCAGGTCTCAGACAAACCGATGGCTTTGTGTCAAACCAAGGCCGCCT |
| | | TCCTCACCTCTGATAAGATGGACGCCTTCTGTCTTCGCGTTTTCAGGCACCCGGGGAAGACCCACAGAACAGGCTAGCTTGT |
| | | TCCCAATTTCCACCTGCTTCCTCCCCATCCCGGACCGACA |
| 532. | S100P | GCTTGGGACATGGTTATCAGCGTCAGATATTTCCTTAAACTAGCGTGAAGTCGACTTTAACCTT |
| | | CCAGCAGTGTGAGTGAGGGCCCTGACCCTAGAATCAACGCCCAAAGAGCTGTGGCGCTACAGCGGGCGGGGACATGGGGTGC |
| | | GCATGCGTGCGGGGGGGTACGCGTGCGTGCAAGTGCGCGTGCGCGCGCAGGACCGCTGAGGCGGGAGCCCCGAATGCGGTTC |
| | | TCGCTTGCTGCGTGGCGGTAAAGGACCGCAGGTGTCGTAAAAGGGCCGCAGTGGCAGCGTCCTGGCCGACGGCTAGTAGCCC |
| | | ATTTTGGATACCGTCCTCGCTGCGGAAAGTTGGGGCAACCTGTTGCTAGTCTGGTCGTTGGTGACAGCGAGGCTTCCGCGCT |
| | | CGCTGCTGGTGAGCAGCCCCGGCGTGCCCCGCGGGCTGGAAGAGGCGGCGGCGTGATGCGGCCCGTGGACGCGGACGAGGCG |
| | | CGGGAGCCCCGCGAGGAGCCGGGCAGCCCGCTGAGCCCCGCGCCCCGCGCCGGCCGCGAGAACCTGGCCTCCCTGGAGCGCG |
| | | AGCGCGCCCGGGCGCACTGGCGGGCCCGCAGGAAGCTGCTGGAGATCCAGAGCCTGCTCGACGCCATCAAGAGTGAGGTGGA |
| | | GGCAGAGGAGCGGGGCGCCCGGGCCCCAGCACCCCGCCCGCGTGCGGAGGCTGAGGAGCGGGTGGCTCGGCTGTGCGCCGAA |
| | | GCAGAGAGGAAGGCTGCGGAGGCGGCGCGGATGGGCAGGCGGATCGTGGAGCTGCACCAGCGGATCGCCGGCTGCGAGTGCT |
| | | GCTGAGCCGGCGAGGCCGCGCGGGTCTGGAGCGGAGCGCGGCGGGGAGTGTCCCGCGTGGAAGGCGCTGGGTAGGCAGGGAG |
| | | GGGAGCGCAGAGCCGTGCCACGCTCTCCGCGGAGTTGGTTTCATTCTTTTTTCATAGGTAATTAGAGAAAATAATTTGATAT |
| | | GTTGTTGTAAAGAGTCATGACCACTGGAAGTATTTTCAGGACGTAGACCTGGTGGGTCACTACGTGGGGGGAGAGAGGTGTG |
| | | AAGCGGAGGTACATGCTGGAAACGTGTGAATGTTGTTACGAGATTGGAGCGTTTGCGATGACCTTCC |
| 533. | SATalpha | GCCACAAAGTGCTCCAAATATCCACTTGCAGGTCCTCCAAAAAGAGTGTTTCAAACGTGAACTA |
| | | CCAAAGGAAGGCTTAGCTCTGGACTTTGAATGCCAACCTCAGAAGGATATTTCTGCGAAAGCTTCTGTTTAGTTAGGCGACG |
| | | TTATCCCGTTTCCAACGAAATCCTCAGAGAGGTCCAAATATCCACCTGCAGAGTCTACAAAAAGTGTGTTTCAAAACTGCTC |
| | | CACCCAAAGGAATGTTCAGCTCTGTGAGTTGAACTCAATCATCCCAAAGTATTTTCTGAGAATGCTTCTGTCCAGTTTTTAC |
| | | ATGAAGCTGTTTCCTTTACTACCGTAGGCCTCATAGCGTTCCAAATCTCCACTTGCAGATGCTACGAAAGGAGCGTTTCAAC |
| | | CTGAACTCACAAGGGAAGGTTCACCTCTGTCAGTTGAATGTCAACATCACAAAGAAGTTCTGAGAATGTTCCTCTTCAGTTA |
| | | CGTGAGGTTTTTCCCGTTTCCAACGAAATTCTCAGAGAAGTCCCAATATCCACTTGCATATCCTACAAAACGTGTGTTTTGA |
| | | AAATGCTCCATCAAAAGAACTGCTCTGCTCTGTGAGTTAAACTCAATCATCGCAAAGAATTTTCTGAGAATGCTTCTGTCTT |
| | | GTTTTTAGATGAAGTTCTTTCCTTTACTACGACAGGCCTCAAAGAGGTCCAAATCTCCACTTGCAGATTCTGCAGAAGGAGT |
| | | GTTTCAAACCTGAACTGTCAGAGAAAGGTTCAACACTGTGAGTTGAATGCAAGCATCACGAAGAAGGTTCTGAGAATGCTTC |
| | | TGTTTACGTAGGTGAGTTCTCTCCCGTATCCAACGAAATCATCAGAGCGGTCCGAATCTCCACTTGCAGATTCTACACAAAG |
| | | TGTGTTTGGAAACTGCTCCATCC |
| 534. | SCAP2 | CTGTCACACACACACACACACACACACACACACCGTCTTCACAATGCACATAGAAACGTCCTGA |
| | | TCCCATTTGTGTAGATCACCAAAAAGGGCTTCGCCACCATCCCCGCACCAAATTTCAACACACACGTCCACTCCCTTTCTGA |
| | | GACAAAACAACCCCTCTCCTCTCCTCCCTGTGCCGACCCCACTGGCTAGAAGACGTGGGAAGCGCGGGGAGGGAGGATAAGG |
| | | GCTCTGAATGCTTCTGTCCCCACCGGCTCACCGTTCCCTCGCCCCCGCCCCGACAGCATTATCGCCGCCTTCCCGCTCTTTA |
| | | CCTGCCAACAGGTTCCTAATTTCCTCAGGGAGGGGGTAGGGAGAGGAGGTGCTGCTGGGGTTGGGCATGTTAGGGAGCGCAG |
| | | GGCGTGCGGGGAAAGGACCTGCGCTGAAAAGGTGACCGACGGGGTGGGGCTGCGGCTGCGACCTAGACTCAGGCTAGCGGCC |
| | | CGGATTAAGAACAGCGGGGCTACGAGTCGGGACACTGCCGGGCCGGGGCTCACAACAAGGAAGTCACTGAATCTCCAGCGAG |
| | | CTGCAGCTGGACTGTCGGCCCAGCCCCGCCCAGAGGGCCGGGGCGGGGAGATGGGTGGGAAGGGACACGAAGGGCCTGAGGG |
| | | GTCCAACTGCGCATGTGTATTCCTCGGTTTTCCCGGCCCCAGAAGAAAGAGCCTGTGGGCAAGCCACGCCCACCGCTACGCC |
| | | GGGAAGCAGGAGGAGGAGCCACTGGTGGGAAGGGGGCGGACTGAGGCTGCGTCACCTGATGCTGCGTCACCTGATCCAGCGT |
| | | CCGGAGCGCCTTACAGCCATTTTCGGTACTGGTGCCATCACAACTGCTGATCGTTTCTGCAGGATTCCAGAAAATAATGTAT |
| | | CCATGATACAGAGTGTATACAAGTTTTGCGATTACGCCTTCTGCGTAACAGTCAGTCCCACCGCAGTTTAACCCACAAGGAA |
| | | TTTTCTCTTGTCCTAAACTATTATGGCCTTCTTTGTCCGATTGAACGGAGGTAACGAAGTCTCGTCTTCCGCCAGTCGTCCG |
| | | GGCGAAGCCAGTCTGGAGCCGCTCCGGAGCGCGCGTTGTGATTGGCTCTTACATTACTTTTCTACCTAATTCGTATCCTTGG |
| | | GGTGACAGATTTTCCCCACTACAAGGAAGACTGGGAAGTTCTAAGCACCGTCCTTTGGCAGGAAAAAACAAACAAAACAAAA |
| | | CAAAAAAACAGAAGGCCGAATAGACATTGGCACCACTGTCCATCTACAAGCATCAAAAATAAAATTGCTGG |
| 535. | SDK2 | CTGGTGACAGCCAGGTAGGTGGAAGTTTGCACCTCGCCGGCTGCATTGCGGGCGAAGCACTGGA |
| | | ACATGCCGGTATCATCGGGCACCAGGCCGCTGATCTGCAGGCCCCCGTCGTTGCGCTGCCGGAAGCGGGTCAACTTCTCCAC |
| | | CTCCACCACGGCTGCGTCCTTGTACCAGGTGATGGAGGGCGGCGGCACACCTGTGGGCAAGACGTGGGCCCACCGTCAACCT |
| | | GCCTCCTGCCTCCTCTCCGCCTAGGAGGGGTGCTTGGGAGGAGGCCGGGCCCGGAAATCTGCGAGGGGGTCCCTGGTGAATC |
| | | TTGGTGTCTAGAACCTTCTCAGAGGTGCCACTTTGGGTCTAGGGAGAGGAGG |
| 536. | SDS-RS1 | TAAATCAGCTGGGCGTGGTGGTGCGCGCCTGTAATCCCACCTACTCAGGAGGATGAGGCAGGAG |
| | | AATCGCTTGAACCCGGGAGGCGGAGGTGGCAATGAGCCGAGATCGCGTCACTGCACTCCAGTCTGGGCGACATATGGAGGTT |
| | | TTGTCTTGTCTCAAAAAAAAAAAAAAAAAAAAACTTGGGGAAGTGTGCTAAACCGGGTTTGCGTAGGTGGAAGGGGCGGCCG |
| | | GGCTCTTTAAGGTGGGTCCCGCCCCGTAGGCCTCCCCTCGCGCCAGCCCCTCCCATCCCGGAAAGGCTCAGCCCAAGCCCGG |
| | | CCCCGCCCGCCCTCGGCCCCGCCCATGGCAGAGCGCAGTCACGTGACCCGGGCTGCGGGGCGCAGCATTGTGCGGTCATGGT |
| | | GGGCCAGATGTACTGCTACCCCGGCAGCCACCTGGCCCGGGCGCTGACGCGGGCGCTGGCGCTGGCCCTGGTGCTGGCCCTG |
| | | CTGGTCGGGCCGTTCCTGAGCGGCCTGGCGGGGGCGATCCCAGCGCCGGGGGGCCGCTGGGCGCGCGATGGGCAGGTCCCTC |
| | | CAGCCTCCCGCAGCCGCTCGGTGCTCCTGGACGTCTCGGCGGGCCAGCTGCTTATGGTGGACGGACGCCACCCTGACGCCGT |
| | | GGCCTGGGCCAACCTCACCAACGCCATCCGCGAGACTGGGTAAGGGTTGGCTTATCCCCACGCGGGGCCATCGGGGGAGGGG |
| | | GATGCGTGGGCGCCGGACCTCGCCTGTTCCCGGGACCGGCCTCTTGCCGGGTGGGAACGCCCGTTTCTCTGGGGGTCAGCTA |
| | | CTCCCGAGGCTGCAGCACCGCCCTGCGGGCCAAGTACCCTGTTTGGGAGCTGGCTACTGCGGCATCCCTCACCTCATGGGGC |
| | | CAGTCCCTTGCCTTCAGCCAGTGGTGACAGTACTTCTGTCCCCAAGAAGCCAGTTCCCTACCACCGAGGCCGCAGTGCCTGC |
| | | ATCTGGATCAGGTCCATGGCTGGTGGGTAAATTCTATCCTCACGGGGACCTGCTCCCTTCCCT |
| 537. | SELENBP1 | AGGGAAGAAGTGACCCTGGCTGATGGGCCCACAGTCCCGGGTGCCCCTAAATGCTAAGAGACAG |
| | | GTCCAAATACAAAGACGAGCATTTGGAGACACAGGAACACATACGTGCGCAGGCTTACACAAGCACATGCACAGCCACATAG |
| | | CCACACACTACACATACACAAGCACTTATGCACATGTCCACACAGGCGTAGATACTCTAGCAAGCACATACTCACAGAGGCG |
| | | CGCACACACACACACACACACACACAGGCGTAGATACTCTGGTAGGCACATACAGAGGCGCGCACACACACACACGCACAAA |
| | | TACACACAAGCACACTCACAGGCACATTCACACACGTGGAAATTCACACACAGGCACAGATACACACTCATGCAGGCACGTG |
| | | CACGAGCAACACGCAGGTATACAGGAACACACACATGCCCAGACACACAAATCCACACACACCCAGGCACCCACTAAAGAGC |
| | | ACGAGCTGGTCAGGGGCAGCGGCTCACACCTGTGATCCCAGCACTTTGGGAGGCCGACACGGGTGGATCACCTGAGGTCAGG |
| | | AGT |
| 538. | SEMA3F | AAAGCAAATGTCCCCTTGAGGAGAGAAGGTGCTAGCTGATCTGGCCTCTCTAAGGTATGGAACT |
| | | ATAAGGAGAGTCTGTGGGAGCTGCTGGCCTGAAATAAGGGCTGGCGATTGAGGCGATCACGGGGAGTCGTTGAATGGCTGGA |
| | | GAGAGCTGTGTGGCTGGGCTGCCCCCGACCTGGCCTAGGCTGGAGGCTGAGCCCGCGGGAAGGTCCGGCGGTGCCAGCCGTC |
| | | CCAGGGGCTGCACCGCCACCTGCTGAGCAGCCCGGGCAGTCAGGGGTCCTGCGCCGCGGGGCCCACGCACGGGACCAGGGGC |
| | | ACGGCCGGCGGCGAGTGGGAATGAGGGACGCGGAAGGGGGTGCTCAGGTGAGCGCAGGCTCGTTGCTCCCTGGCGCTGCAGC |
| | | CCTTCTCGCCAGGGAGTTGAATGCCCGGGTAAACACCAGGCGCGATGTGTGCGGTGTATTCACAGAGAGACAAAGCCAGGGC |
| | | GGGGCGGGGGCGCCGCCCGCGAAGCGGGGGCGGGGGCGAGGGGTGCAGACTAGTCTCCCCCGGCGCGGGGTGCCCACCGCCT |
| | | GGGAACCCTGAGCCCGGCCCACGGACAGGTGGAGCGGGGGCGGGGCGGTGGCTGGGCGGGTCCCGCCCCGGGGGTGGGGCTG |
| | | GGCGGGCCGGGCGGGGCGGGGCTGCGCTATGCAAATGTCGCCCACGGGCGGCCAATTGCCGGCGCTCCCCGCGCGGCTCTGA |
| | | GCGCCCCGTCCCGCCGGCGGCCGCGAGACCAGAGCGAGCGAACGAACCGCGGCGGTCCGGAGAGCCCCGAGCGCAGCGCAGG |
| | | ACCTGGGTACGCCGCGAGGAACCGTGCAGCCCAGCGCGGCCGCCCGGCCCGGGTCCAGCAGCCAGGAGAGCGCAGCGCTTCG |
| | | AAGCCGAGTGCGCGCCACCGCCCGCGCCCCGCGCTGGGGAATGCGCCCTCGGCGCGCCGGCCAGGGGGCGCCCGCAGCCCAC |
| | | CCCAGGGGAGGCGGCCCCGAGCGCCCCTGAGCCTTCCCATGGCCCGGGCTGGGGCCCGGGCCCTCGGCTGCTGACGCGCCCG |
| | | AAGCCCGCGGAACCGGTTAAGCCGCGGCCGCGGCGCCGATCCCGGCTGAGGCGCAGCGGCGAGAGGTCGCGGGCAGGGCCAT |
| | | GGCCCCGGGGGGCCGCTAGCGCGGACCGGCCCAACGGGAGCCGCTCCGTGCCGCCGCCGCCGCCCGGGCGCCCAGGCCCCGC |
| | | CGCTGCGGAAGAGGTGAGTGCAGCGGGAACCGGGAGGGAGCGGGCAGGCGGCCGGGCCACCCCGCGACCCCTCTGGGACCCG |
| | | CGGCACTGCAACTCCGCAGAAGTGTCCGGGGAGCGGGTCTCGTCGAGCCGGGGGGCTGCCCGCGGACATAGGGGCAACAAGG |
| | | CTGGGGTGGGATTCTTACCTGTCCTGGAGGCCCGGACCCCTTACCTACGGGGCGGCGTATGGATGTGTGGATGATGTGGCCT |
| | | GCGGGGTCACCCATGTGCAAAGCAACTTTTTCCTGCAAGGTTCTGGGTGGGTGCTCTCATACACCCCCACCTTCACGCTTCT |
| | | GGAGCGGGAGTTCCGATC |
| 539. | SERPINA3 | GGGAGGCTGAGGCAGAAAGTTGCTTGAACCCGGGAGTCAGAGGTTGCAGTGAGCTGAGATCACG |
| | | CCACTGCACTCCAGCCTGGGCAACAAAGCAAGACTCTGTCTCAAAATAAAAATAAAAAATAAAAAGAAATAAAAAAGAAATA |
| | | TACCAAAATGTTAGCTGGGGTCTTCTCTGGGTAGTAAAGTGCTGGGGGATATTTTCCAAAGTCCTTCTTTACATTCTCTGAG |
| | | TTTTTCCATGTTCTTCAATGAGTATTTAATAAGCAGATAAAAACTAATACAACAAAGGATTTTTTCTGTGTGCTTTTTTGAC |
| | | CTTTGGAGGAAGAGATTAGAGCTAGTCCCATAACCAGGTTATTTGAGTAGGTCTAACAAGCCCGTATTACCAGAAATTATCA |
| | | TCTGGTCATTTCCAGTCCGAGAACAGAACACTTGGTTGTCCTGGCATTTCCCAAGCAGGGGGAGGAGTTCTCTGCAGGAATA |
| | | AATAAGCCTCAGCATTCATGAAAATCCACTACTCCAGACAGACGGCTTTGGAATCCACCAGCTACATCCAGCTCCCTGAGGC |
| | | AGAGTTGAGAACACGTTGCTTGTGCCTTTCCATGCTGACTATCAGACTCTGCGCCCCTGAGGCTACCTGTGCCCCAAAGACA |
| | | AGCCCCTTGAGCTCCCATGCAGGCTCAGCTCCACCCCAAGATCTGGGCTCCTTCTGCCTTCAGCTGCAGCAGCCTGCCCAGG |
| | | ACTACCTAAGCTTGTGGCCAAGCTCACCCAGACCTCAGGCCCAGGGTTGGGAGGGGACCCTGCTGCTCTAAGGCCAATTCCT |
| | | GCCCCCCAGGCTTCTGGGTGCCCACTTTCAGTTTCTCTCCTTCCACCCCCAGGGCCCAGGCTCCACCTGGCTCTTTCCAGAG |
| | | CTCCTAGCCCTGACCACATCTGGCACAGCCCCCGGACTCCTTGCTCCTGCAGCTGTGCAGGGCTTTATCATGCGACATCCTT |
| | | CTTCTGCCCTTTAGCACCCCCAATTTGGGGGAAGTAGCTACTTTCTGGGTTTCCAGATGGAGCCCCTCAACTGCCTCACCAG |
| | | TTCCTAGCCCTGGCTCTG |
| 540. | SERPINA5 | GGGAGGCTGAGGCAGAAAGTTGCTTGAACCCGGGAGTCAGAGGTTGCAGTGAGCTGAGATCACG |
| | | CCACTGCACTCCAGCCTGGGCAACAAAGCAAGACTCTGTCTCAAAATAAAAATAAAAAATAAAAAGAAATAAAAAAGAAATA |
| | | TACCAAAATGTTAGCTGGGGTCTTCTCTGGGTAGTAAAGTGCTGGGGGATATTTTCCAAAGTCCTTCTTTACATTCTCTGAG |
| | | TTTTTCCATGTTCTTCAATGAGTATTTAATAAGCAGATAAAAACTAATACAACAAAGGATTTTTTCTGTGTGCTTTTTTGAC |
| | | CTTTGGAGGAAGAGATTAGAGCTAGTCCCATAACCAGGTTATTTGAGTAGGTCTAACAAGCCCGTATTACCAGAAATTATCA |
| | | TCTGGTCATTTCCAGTCCGAGAACAGAACACTTGGTTGTCCTGGCATTTCCCAAGCAGGGGGAGGAGTTCTCTGCAGGAATA |
| | | AATAAGCCTCAGCATTCATGAAAATCCACTACTCCAGACAGACGGCTTTGGAATCCACCAGCTACATCCAGCTCCCTGAGGC |
| | | AGAGTTGAGAACACGTTGCTTGTGCCTTTCCATGCTGACTATCAGACTCTGCGCCCCTGAGGCTACCTGTGCCCCAAAGACA |
| | | AGCCCCTTGAGCTCCCATGCAGGCTCAGCTCCACCCCAAGATCTGGGCTCCTTCTGCCTTCAGCTGCAGCAGCCTGCCCAGG |
| | | ACTACCTAAGCTTGTGGCCAAGCTCACCCAGACCTCAGGCCCAGGGTTGGGAGGGGACCCTGCTGCTCTAAGGCCAATTCCT |
| | | GCCCCCCAGGCTTCTGGGTGCCCACTTTCAGTTTCTCTCCTTCCACCCCCAGGGCCCAGGCTCCACCTGGCTCTTTCCAGAG |
| | | CTCCTAGCCCTGACCACATCTGGCACAGCCCCCGGACTCCTTGCTCCTGCAGCTGTGCAGGGCTTTATCATGCGACATCCTT |
| | | CTTCTGCCCTTTAGCACCCCCAATTTGGGGGAAGTAGCTACTTTCTGGGTTTCCAGATGGAGCCCCTCAACTGCCTCACCAG |
| | | TTCCTAGCCCTGGCTCTG |
| 541. | SERPINB5 | GCTGTGGGGTGGGGCACACTTGGTTGGGGGCGGACGGGGGTTTAGGAGAACGGCGAGGAGCGGG |
| | | CGCAGAAGGGTGGCGGGGCCGCCGGGTCGTTGCGCGGCAGTTGCGTCCGGCCTCTTGCGCGCGGCGCCCGGGAAGGGGCGGG |
| | | GCCGAGGCGGGCAAGGTGGCGGGCCCCCGCCCCTGGCCCCGCCCCCGCAGCCCGCCCGCGAGCCTCGCCCCGCCTCCTCGCC |
| | | GGGCCCGCCCCGCCCCCTCGCCGGGCCGTGCTCTTGCTCCCGCCGCCTGGCAGCCTCACGCTCGGCTCCAGCGGCCAAGAGC |
| | | CGGAGAAAGTCCTGCTGGTGGGCGGCCGCGGGGCTGAGGGCGTCCGGCATCCCGGGGCCGCTCCGGCCCGGGCGGCGAGAGT |
| | | GCCCGGCGGTCCATGCATCCGCCGCCGCCCGCCGCCGCGATGGATTTCAGTCAGAACAGCCTGTTCGGTTACATGGAGGACC |
| | | TGCAGGAGCTCACCAT |
| 542. | SFTPB | CGGTTTAGCAGGGCGCCTGGAGTGAGGTGACTGCGGAGGCTCGCAGACGTTAGGTCTGCCTAAA |
| | | TCCGAAGCTTCCACCCTCCTCTGCCTCTGTGACTTGCTGCGTGACTTTGGAGCTTTCGGAAACTCAGTTTCCCTGTCTTAAG |
| | | CCCTCTGCTCCTCTTGCTTTCCCGCTCCAGCAAGTGAGAGTGGACTGGGTTGCCGTGCCGGGCGGGTGTGGGTCGCCGGGCA |
| | | ACTCCGGAGTCTCCCCTTCCCCATCGGCCCCCAGCAGAAGTCCCAGCTCCCGGCGTTTTCTGTCTCGTGGCGAGTTGGGGCG |
| | | GAGGGAGCGGCGGGAAGGTGCCGGGTGGGCAAGGTCGGAGCTGCGTCACCGAGAGGCCGGCAAGGCCAGGGAGAAAAGGCCC |
| | | CGCTGTGATTTGGGGAAGGGCCGGGGGCCCATAAGTCACGTGCTCGGGGCGGTTGTGCAGGAGCGGACTGTCCTCTGGGAAC |
| | | ATAGAAGCGCCCCCGACTAGAGTAGGGGCGGGGAGGGAGCGCCGGAGGAGGCAGGTTCTGGGCCAAGGGGATGGGGGTGGGG |
| | | GGGAGGTAGGGAGCCCGCGGACAAAGGAGGCGGCCGGCGGCCCAGCTGTTTTGAAAAATGCTTCCTGTTTCTTTAAAGGCGC |
| | | TCGCGGCTCGGGCGGCCCGGGCTGGGGAGGCGGTGGCGGCGGGAGCTGCCTCCTCTCCGGGCGGCGGCGCTGACTGATCTCG |
| | | CGAACTGGGCTTCTGTGTAAACTGAGGCTAAACAAACACAGATGGAGCGCAGCGGGCGTTCTCCAGAAATGCTGGCAAGGAG |
| | | GCAGCGACTTCAGATGACACTCTGAGCGCTCCGGGAACGGACAGCCCGGCGGCTTCCCGAAGCCGGCGGCGCAGCTGCCCGG |
| | | GGCGAGGGGGAGAAAGGGAGAGAGGGAGGGGGAGGGCGGGCGAAGCGGGAGAGCCAGAGACTCCTCGGCGCTGAGCGCGGCG |
| | | GCGGCCCGGGCAGCCCCACGCCCCTGCCTCGCGCGCCGCCCGCGCCATGAAGCACATCCCGGTCCTCGAGGACGGGCCGTGG |
| | | AAGACCGTGTGCGTGAAGGAGCTGAACGGCCTTAAGAAGCTCAAGCGGAAAGGCAAGGAGCCGGCGCGGCGCGCGAACGGCT |
| | | ATAAAACTTTCCGACTGGACTTGGAAGCGCCCGAGCCCCGCGCCGTAGCCACCAACGGGCTGCGGGACAGGACCCATCGGCT |
| | | GCAGCCGGTCCCGGTACCGGTGCCGGTGCCAGTCCCAGTGGCGCCGGCCGTTCCCCCAAGAGGGGGCACGGACACAGCCGGG |
| | | GAGCGCGGGGGCTCTCGGGCGCCCGAGGTCTCCGACGCGCGGAAACGCTGCTTCGCCCTAGGCGCAGTGGGGCCAGGACTCC |
| | | CCACGCCGCCGCCGCCGCCGCCTCCTGCGCCCCAGAGCCAGGCACCTGGGGGCCCAGAGGCACAGCCTTTCCGGGAGCCGGG |
| | | TCTGCGTCCTCGCATCTTGCTGTGCGCACCGCCCGCGCGCCCCGCGCCGTCAGCACCCCCAGCACCGCCAGCGCCCCCGGAG |
| | | TCCACTGTGCGCCCTGCGCCCCCGACGCGCCCCGGGGAAAGTTCCTACTCGTCAATTTCACACGTAATTTACAATAACCACC |
| | | AGGATTCCTCCGCGTCGCCTAGGAAACGACCGGGCGAAGCGACTGCCGCCTCCTCCGAGATCAAAGCCCTGCAGCAGACCCG |
| | | GAGGCTCCTGGCGAACGCCAGGGAGCGGACGCGGGTGCACACCATCAGCGCAGCCTTCGAGGCGCTCAGGAAGCAGGTACCC |
| | | GCTCGCCGCCGCACGCCCTCACTGCGCCGGGGGACGACTGCGGGAATGGGTGGGCGAGTGGCCGGGGCGGGATAGAGGTGTG |
| | | TTTAAGGGGCAAGCTGCCCCGCCCGGTCCGACCCTGGTGCCCAGACAGGTGTGGGCAGTCCTAGGGTATGGATCATAGTTTT |
| | | CAAAGTGGCTATAAGAGTTGGTTATCTCTGGCCCGAGCACCCCCCGCCCTCAACCCCCGCCTCGGGGGTAACGCTGTCTGCG |
| | | CTTCTGACTTCAGCCTCCATAGTTCACTTTTATGGCAGCGAGTATTTGCCTGCCGGAGTCCCCTGG |
| 543. | SLC2A1 | AGACTAGGGGGAGGAAAGGAGGTGGAAAGAAACAATGCCCTACACACAGGAGGGGCTCCACAAA |
| | | CAATTTGTTGAAAAGAAATCGCTCTGCGGACGCTACAAAGATGCCCTGCAGGACAAGCCCACTTACACAGAAAAAAAGAAAT |
| | | CCTATCTAGGGAGTTGTTGGTCCCCAGAGTCCCCATCCCTCATTCCCCAACCCCGTGGCGTGCGCGCTTCCCGCTAGACTTC |
| | | AGCCTGGGGCTCTTTCCTGGCCCCCAGATCCCCCGCCCCTCCGGTTGCGCTCCCCGCCTGGCCCCGAGGGTAAACCTGCCTC |
| | | CGCGGAACACATCTCCGCGAGACTGGGCCAAAGCTTTGGGGCCTGCACTCCTCGCAAAGGAGAGCCTCCGAGACCGGAGGTT |
| | | CATCTCAACCCCCGCGTTCGAGCCCGGGCCTGGAGAAAAGTGCCCTCCCCTCGGCCTCGGCCTCGTCACCGCGGGGGACTGG |
| | | CGGGAGGAGTAGGGGAGGCCGGTGGGACACCTCGCACCAGGCCGCCCACCTACAGGGGCGTCCGGGCTAGGGGAGCAGACGG |
| | | AGAGCGGGGGCGGCTCCTGACTCCTCCGCGCGCCGGGGCCGGGGCCGGGCCGGGGGCTCGGCCCGCTCAGCCAATGGGCGCC |
| | | GCGCTCGGGCCCCCTCCCCCGCACCGGGAGGGGCCGAGGCTGGGCTGCTGCTGCCGAGGTCGTGTGCGACGTGCCGCTCCCG |
| | | GGCCCTGTGTCCCCAGCCCCAGCAGGGCCGCGTGGACGGCGCGGCGCCCCCGGAAGGAGGCCATGTGCTCAGTGCCGGCGCC |
| | | AACTACGCACGCGGCGCCCTCCGGCCCGCTAGATCCGAAGCCCATCCCCACTGCGGCCAGCGGCCCGGCGGGGCGCTGCGCT |
| | | CGGGACCCGCACCGAGCCAGGCTCGGAGAGGCGCGCGGCCCGCCCCGGGCGCACAGCGCAGCGGGGCGGCGGGGGAGGCCCT |
| | | GGCCGGCGTAAGGCGGGCAGGAGTCTGCGCCTTTGTTCCTGGCGGGAGGGCCCGCGGGCGCGCGACTCACCTTGCTGCTGGG |
| | | CTCCATGGCAGCGCTGCGCTGGTGGCTCTGGCTGCGCCGGGTACGCGGGTGGCGACGGGCGTGCGAGCGGCGCTCTCCCGCT |
| | | CAGGCTCGTGCTCCGGTCCGGGGACTCCCACTGCGACTCTGACTCCGACCCCCGTCGTTTGGTCTCCTGCTCCCTGGCGTGC |
| | | TCACTCGGGGACCCGCGACTAGCGACCGGCACGCTCGCTGTTGCTACCTCTTGCCTCTTGCCAGAGAGCGCGCGGACCGTAG |
| | | CGTTTATAGGACCCCGGCCATTGGCTGGCGACGCCGGTGTGTCAGGGGTGTGTGGGCAGGACCTCGGGGCGGGGCCTGGGGC |
| | | CCAGCCTGTCCTGGGCGGCGCTCTGCGGGGAGGGGGTGAGGGGAGTGGCCGGGGCCGGGGCCCCCCCTAACCACTGCCTCGC |
| | | TCAGGCTGCCGATCGGCTCGTTCTCTCTGCGTTGGGACCGCGGAGGGCATTCGCTGTGTGACTCAGGGCAAGTGTGCGCACC |
| | | TCTCTGAGCCTCCGCGGCCTTCTCTAGCAAAATGGTGGAGCCGGTACCCGGCTGTAAGGCAAGCTGGGATGCGCGGAGTAGC |
| | | GGGTGGAAAGCTGGACTGGAGTTCTCACTCGGCCGCTGGGTGACTTCGGTGCACTAGGGATAGTAACAGTACCACCTC |
| 544. | SLC6A8 | ACGATCACACACGCGCGCGCACACTCACGAGCCGAGCCTCTCGCGGATCTCGTAGACGCTGCTG |
| | | ATGTCCTCCGTGTGTTTCTTCAGCAGCAGCATGTCTGCAGGGGCAGGGGAGAGGTGATGGGGGTCTCTCCCCGCCCCGCCGG |
| | | GGGCAAGGGCAGCTTCCTCCTCCACCCCCTGCCAGGCAGGCCTCAGCCATTGCCGCTGCGCGCTCGACCCTCCCCTCCCCCG |
| | | TCCAGGTCCACACCCCCAGACGGACGCCTCCCAGCGAGGCCACCGCATACACGCCTCCACATCCCAGGCCTACAGCTGCTAT |
| | | CCCACGCACCCTCCCCCGCACCCCGAAGGCGACCAGCGCCTGGATGCTGCCTCTCTGCCTCCACACATCCGGCCGCTTTCCA |
| | | GAAGGCTCCATGCTCACCCACGCCGTCCCTCCCCCATCCACACATCTACAGCGCCCAAGCCCCCACCCCCGCCTAGACCCGC |
| | | CACCCATGGCACAGCGCCCACAGGCACCCGCTGCCCAAGCCACCAAGCCGCCCGGGATGCACCTGCGACCCCCGCGCGCAGC |
| | | CACCTGCCAAGACCGCGGCCACCCACGCGGCCGCAGACGCAGCCCAGCCCTCGGCGCCGCGGAGCAGGGGGACCTGGCAGCC |
| | | TGGGCCGCCCGCGTCCCGAGCTGCGTGGACCGAGCGCGTGCGGGCAGGGGGCGCGCCAGGCGTGACAGGTGTGCTGCGGACG |
| | | CGCAAGGCTGCGGCGGCCGCGGGCCGGATCCGGTGCGCCCCCGCCCCGCGCGTCCCCGCGCCCCGGAGCAGGTGCAGACCTG |
| | | GAGCGGGTGCCGCAGCGTCGTGCCGCGCAGTGGCCGCAGCGCCAAGCCCCCCGCCCGCGCCCCGCCGGCCCCGCCCCCTCCG |
| | | CGCACGCCCCCGCCAGCCGCCCAAGCACGCAGTGGCCCAGCGCGGCCGCGGCGCCGCAGGGCACCTTGTAAGGCAATCGCCG |
| | | GGATCCGGCTCGGGTGCCGGCCGCCACCGCCCAGCCCACTGCTCGCTCGCGCCGCCTCCCGGAGCTGCGGCCCGGCTGGGCC |
| | | GGCCCCAGGGCCTTTGCACCACCGGTGGCTGTCTCCCGGAGAGCTCTTCGGCCACAGGTCGGCAAGGCTCTCTGCCTTCCAG |
| | | TCTCTGCCTTCACCGCCTCTCCGAGGACAGGCTGACCCGGCCACTTGCCCGAAAGCAGCCTCCATGCCTAGCTCCAGGACCC |
| | | TGCAACCCTGCACCACAGCTCTGCCACCCTGGGCTCCCGGCACTCACTGGGCTCCCTCTGGCTCCACCATTAATCCCTCACC |
| | | CTCGCTTTCTTCTCT |

TABLE 9
FIG. 6A x-axis: CpG sites (left to right)

X053_KIAA1447_01_CpG_2.3.4
X053_KIAA1447_01_CpG_5
X053_KIAA1447_01_CpG_6
X053_KIAA1447_01_CpG_7.8.9.10
X053_KIAA1447_01_CpG_15.16
X053_KIAA1447_01_CpG_17
X053_KIAA1447_01_CpG_18
X053_KIAA1447_01_CpG_19
X053_KIAA1447_01_CpG_20.21.22.23.24
X053_KIAA1447_01_CpG_26
X096_SLC6A8_01_CpG_1.2.3.4
X096_SLC6A8_01_CpG_7
X096_SLC6A8_01_CpG_10
X096_SLC6A8_01_CpG_12
X096_SLC6A8_01_CpG_13
X096_SLC6A8_01_CpG_15
X096_SLC6A8_01_CpG_16.17
X096_SLC6A8_01_CpG_20
X096_SLC6A8_01_CpG_21
X096_SLC6A8_01_CpG_22
X096_SLC6A8_01_CpG_23
X096_SLC6A8_01_CpG_24
X096_SLC6A8_01_CpG_25.26
X096_SLC6A8_01_CpG_27
X096_SLC6A8_01_CpG_28.29.30
X096_SLC6A8_01_CpG_31.32
X096_SLC6A8_01_CpG_33.34.35
X096_SLC6A8_01_CpG_37.38.39.40
X019_COL1A1_01_CpG_1.2
X019_COL1A1_01_CpG_3
X019_COL1A1_01_CpG_4.5.6
X019_COL1A1_01_CpG_7
X019_COL1A1_01_CpG_8.9
X019_COL1A1_01_CpG_11
X019_COL1A1_01_CpG_12
X019_COL1A1_01_CpG_13.14.15
X019_COL1A1_01_CpG_16
X114_ZD52F10_16_CpG_5
X114_ZD52F10_16_CpG_6
X114_ZD52F10_16_CpG_7.8
X114_ZD52F10_16_CpG_11
X114_ZD52F10_16_CpG_14
X114_ZD52F10_16_CpG_15
X114_ZD52F10_16_CpG_16
X114_ZD52F10_16_CpG_17.18
X114_ZD52F10_16_CpG_20.21
X114_ZD52F10_16_CpG_22
X114_ZD52F10_16_CpG_23
X114_ZD52F10_16_CpG_24.25
X114_ZD52F10_16_CpG_27
X114_ZD52F10_16_CpG_28
X114_ZD52F10_16_CpG_29.30
X114_ZD52F10_16_CpG_31.32.33
X114_ZD52F10_16_CpG_34.35
X114_ZD52F10_16_CpG_38
X114_ZD52F10_16_CpG_39
X102_STX1A_04_CpG_1
X102_STX1A_04_CpG_2
X102_STX1A_04_CpG_3
X102_STX1A_04_CpG_5
X102_STX1A_04_CpG_6.7.8
X102_STX1A_04_CpG_9.10.11
X102_STX1A_04_CpG_12
X102_STX1A_04_CpG_13
X102_STX1A_04_CpG_14.15.16
X102_STX1A_04_CpG_17.18.19
X102_STX1A_04_CpG_22
X102_STX1A_04_CpG_23
X102_STX1A_04_CpG_24
X102_STX1A_04_CpG_25
X102_STX1A_04_CpG_26
X102_STX1A_04_CpG_28
X102_STX1A_04_CpG_29
X102_STX1A_04_CpG_30.31.32
X102_STX1A_04_CpG_35
X102_STX1A_04_CpG_36
X102_STX1A_04_CpG_37
X102_STX1A_04_CpG_38
X102_STX1A_04_CpG_39
X130_CDX2_32_CpG_1.2
X130_CDX2_32_CpG_3.4
X130_CDX2_32_CpG_10.11.12
X130_CDX2_32_CpG_13
X130_CDX2_32_CpG_14.15
X130_CDX2_32_CpG_16
X130_CDX2_32_CpG_22.23.24.25
X130_CDX2_32_CpG_27.28
X130_CDX2_32_CpG_29
X130_CDX2_32_CpG_30.31
X024_DUSP4_01_CpG_1
X024_DUSP4_01_CpG_2
X024_DUSP4_01_CpG_4.5.6.7
X024_DUSP4_01_CpG_8
X024_DUSP4_01_CpG_9
X024_DUSP4_01_CpG_10.11.12
X024_DUSP4_01_CpG_13
X024_DUSP4_01_CpG_14
X024_DUSP4_01_CpG_15
X024_DUSP4_01_CpG_16
X024_DUSP4_01_CpG_17.18
X024_DUSP4_01_CpG_19.20
X024_DUSP4_01_CpG_21
X024_DUSP4_01_CpG_22
X024_DUSP4_01_CpG_23
X024_DUSP4_01_CpG_24
X024_DUSP4_01_CpG_25
X002_E.cad_02_CpG_1
X002_E.cad_02_CpG_2.3.4
X002_E.cad_02_CpG_5.6.7
X002_E.cad_02_CpG_8
X002_E.cad_02_CpG_15
X002_E.cad_02_CpG_16.17
X002_E.cad_02_CpG_19.20.21
X002_E.cad_02_CpG_22
X002_E.cad_02_CpG_23
X002_E.cad_02_CpG_24
X154_RARB_56_CpG_1
X154_RARB_56_CpG_3
X154_RARB_56_CpG_4
X154_RARB_56_CpG_5
X154_RARB_56_CpG_6.7
X154_RARB_56_CpG_10.11
X154_RARB_56_CpG_12
X154_RARB_56_CpG_14
HOXA9_SQ03_CpG_1
HOXA9_SQ03_CpG_2
HOXA9_SQ03_CpG_3
HOXA9_SQ03_CpG_4
HOXA9_SQ03_CpG_5.6
HOXA9_SQ03_CpG_8.9
HOXA9_SQ03_CpG_11.12.13.14.15.16
HOXA9_SQ03_CpG_17.18
HOXA9_SQ03_CpG_20
HOXA9_SQ03_CpG_21
HOXA9_SQ03_CpG_22.23.24.25.26
HOXA9_SQ03_CpG_28
HOXA9_SQ03_CpG_29.30
HOXA9_SQ03_CpG_31
HOXA9_SQ03_CpG_32.33
HOXA9_SQ03_CpG_34.35.36.37
HOXA9_SQ03_CpG_38.39
HOXA9_SQ03_CpG_40
HOXA9_SQ03_CpG_41
HOXA9_SQ03_CpG_42.43.44.45.46
X005_HOXA9_AB02.2_CpG_1.2.3.4
X005_HOXA9_AB02.2_CpG_5.6.7
X005_HOXA9_AB02.2_CpG_8.9
X005_HOXA9_AB02.2_CpG_12
X005_HOXA9_AB02.2_CpG_13
X005_HOXA9_AB02.2_CpG_15.16
X125_C10orf38_27_CpG_4
X125_C10orf38_27_CpG_8
X125_C10orf38_27_CpG_9
X125_C10orf38_27_CpG_10
X125_C10orf38_27_CpG_11
X125_C10orf38_27_CpG_13
X125_C10orf38_27_CpG_15
X125_C10orf38_27_CpG_16.17
X125_C10orf38_27_CpG_18.19
X145_LRP6_02_CpG_1
X145_LRP6_02_CpG_4.5
X145_LRP6_02_CpG_7.8.9.10
X145_LRP6_02_CpG_12
X145_LRP6_02_CpG_14
X145_LRP6_02_CpG_30
X145_LRP6_02_CpG_31
X145_LRP6_02_CpG_39.40
X145_LRP6_02_CpG_41.42
X145_LRP6_02_CpG_43.44
X145_LRP6_02_CpG_45
X145_LRP6_02_CpG_46.47.48
X145_LRP6_02_CpG_49.50.51
X145_LRP6_02_CpG_53
X145_LRP6_02_CpG_54
X145_LRP6_02_CpG_55
X145_LRP6_02_CpG_56
X033_FLJ23058_01_CpG_7
X033_FLJ23058_01_CpG_11.12.13.14
X033_FLJ23058_01_CpG_18
X033_FLJ23058_01_CpG_20.21
X033_FLJ23058_01_CpG_22
X033_FLJ23058_01_CpG_23
X033_FLJ23058_01_CpG_24
X033_FLJ23058_01_CpG_25
X033_FLJ23058_01_CpG_26.27
X033_FLJ23058_01_CpG_28
X033_FLJ23058_01_CpG_29.30
X033_FLJ23058_01_CpG_31
X033_FLJ23058_01_CpG_32
X033_FLJ23058_01_CpG_34
X033_FLJ23058_01_CpG_35
X033_FLJ23058_01_CpG_37
X033_FLJ23058_01_CpG_38
X033_FLJ23058_01_CpG_39
X033_ACTG1.03_CpG_1
X033_ACTG1.03_CpG_3.4.5
X033_ACTG1.03_CpG_6.7
X033_ACTG1.03_CpG_8
X033_ACTG1.03_CpG_9
X033_ACTG1.03_CpG_10
X033_ACTG1.03_CpG_11
X033_ACTG1.03_CpG_12.13.14
X033_ACTG1.03_CpG_15
X033_ACTG1.03_CpG_17
X033_ACTG1.03_CpG_18
X033_ACTG1.03_CpG_19
X033_ACTG1.03_CpG_20
X033_ACTG1.03_CpG_21
X033_ACTG1.03_CpG_22
MYOD_01_02_CpG_1.2
MYOD_01_02_CpG_4
MYOD_01_02_CpG_5.6
MYOD_01_02_CpG_7.8.9
MYOD_01_02_CpG_10
MYOD_01_02_CpG_11
MYOD_01_02_CpG_12.13.14
MYOD_01_02_CpG_16.17
MYOD_01_02_CpG_18
MYOD_01_02_CpG_22.23
MYOD_01_02_CpG_24
MYOD_01_02_CpG_25.26
MYOD_01_02_CpG_27.28
MYOD_01_02_CpG_29.30.31
MYOD_01_02_CpG_32
MYOD_01_02_CpG_33
MYOD_01_02_CpG_34.35.36
MYOD_01_02_CpG_37.38.39
MYOD_01_02_CpG_42.43.44
MYOD_01_02_CpG_45
MYOD_01_02_CpG_46.47
X134_DLK1_36_CpG_11
X134_DLK1_36_CpG_12
X134_DLK1_36_CpG_18
X012_ACTG1.09_CpG_1
X012_ACTG1.09_CpG_2
X012_ACTG1.09_CpG_3
X012_ACTG1.09_CpG_4.5.6
X012_ACTG1.09_CpG_8.9
X012_ACTG1.09_CpG_10.11
X012_ACTG1.09_CpG_12
X012_ACTG1.09_CpG_13
X012_ACTG1.09_CpG_14
X012_ACTG1.09_CpG_15.16
X012_ACTG1.09_CpG_17
X082_PRG2_01_CpG_3
X082_PRG2_01_CpG_4
X082_PRG2_01_CpG_8.9.10
X082_PRG2_01_CpG_11
X082_PRG2_01_CpG_12
X082_PRG2_01_CpG_16.17.18.19
X082_PRG2_01_CpG_20.21
X082_PRG2_01_CpG_22
X082_PRG2_01_CpG_30
X082_PRG2_01_CpG_31
X082_PRG2_01_CpG_32.33.34
X14_RUNX3_01_01_CpG_1
X14_RUNX3_01_01_CpG_7
X14_RUNX3_01_01_CpG_9.10.11.12
X14_RUNX3_01_01_CpG_17
X14_RUNX3_01_01_CpG_22.23.24
X14_RUNX3_01_01_CpG_29
X14_RUNX3_01_01_CpG_32
X017_CKMT1_01_CpG_5
X017_CKMT1_01_CpG_6
X017_CKMT1_01_CpG_7
X017_CKMT1_01_CpG_8
X017_CKMT1_01_CpG_9
X017_CKMT1_01_CpG_10
X017_CKMT1_01_CpG_11.12
X017_CKMT1_01_CpG_13
X017_CKMT1_01_CpG_15
X017_CKMT1_01_CpG_16
X017_CKMT1_01_CpG_17
X017_CKMT1_01_CpG_22
X017_CKMT1_01_CpG_23
X017_CKMT1_01_CpG_24.25
X017_CKMT1_01_CpG_30
X017_CKMT1_01_CpG_31.32
X017_CKMT1_01_CpG_33
X017_CKMT1_01_CpG_35
X017_CKMT1_01_CpG_36
X024_ACTG1.01_CpG_2
X024_ACTG1.01_CpG_3
X024_ACTG1.01_CpG_5
X024_ACTG1.01_CpG_6.7
X024_ACTG1.01_CpG_8
X024_ACTG1.01_CpG_9
X024_ACTG1.01_CpG_11.12
X024_ACTG1.01_CpG_14.15
X024_ACTG1.01_CpG_16
X024_ACTG1.01_CpG_17.18.19
X024_ACTG1.01_CpG_20
X024_ACTG1.01_CpG_21.22.23.24.25
X024_ACTG1.01_CpG_35.36.37.38
X024_ACTG1.01_CpG_46
X024_ACTG1.01_CpG_47
X024_ACTG1.01_CpG_48.49
X024_ACTG1.01_CpG_50
X024_ACTG1.01_CpG_51
X024_ACTG1.01_CpG_53
X005_ERalpha_02_CpG_2.3
X005_ERalpha_02_CpG_4.5
X005_ERalpha_02_CpG_6.7
X005_ERalpha_02_CpG_8
X005_ERalpha_02_CpG_9.10
X005_ERalpha_02_CpG_12.13.14.15.16
X005_ERalpha_02_CpG_17.18
X005_ERalpha_02_CpG_19
X005_ERalpha_02_CpG_20
X005_ERalpha_02_CpG_21.22
X005_ERalpha_02_CpG_23.24.25
X005_ERalpha_02_CpG_26
X005_ERalpha_02_CpG_27.28
X005_ERalpha_02_CpG_29
X005_ERalpha_02_CpG_30.31.32
X005_ERalpha_02_CpG_33
X005_ERalpha_02_CpG_34
X018_CNN3_01_CpG_1.2.3
X018_CNN3_01_CpG_10.11.12
X018_CNN3_01_CpG_13
X018_CNN3_01_CpG_22
X018_CNN3_01_CpG_23.24
X018_CNN3_01_CpG_30
X018_CNN3_01_CpG_35.36.37
X018_CNN3_01_CpG_42.43
X018_CNN3_01_CpG_50.51
X018_CNN3_01_CpG_53.54.55
X018_CNN3_01_CpG_56.57.58
X018_CNN3_01_CpG_59
X077_PBX3_01_CpG_1
X077_PBX3_01_CpG_2
X077_PBX3_01_CpG_3
X077_PBX3_01_CpG_4.5.6.7
X077_PBX3_01_CpG_8
X077_PBX3_01_CpG_10
X077_PBX3_01_CpG_11.12
X077_PBX3_01_CpG_13.14.15
X077_PBX3_01_CpG_16
X077_PBX3_01_CpG_17.18
X077_PBX3_01_CpG_21.22
X077_PBX3_01_CpG_23
X077_PBX3_01_CpG_24.25
X077_PBX3_01_CpG_35
X077_PBX3_01_CpG_36.37.38
X077_PBX3_01_CpG_39.40.41
X077_PBX3_01_CpG_42.43
X095_SLC2A1_01_CpG_4.5
X095_SLC2A1_01_CpG_6
X095_SLC2A1_01_CpG_7
X095_SLC2A1_01_CpG_8.9.10
X095_SLC2A1_01_CpG_11.12
X095_SLC2A1_01_CpG_13
X095_SLC2A1_01_CpG_14
X095_SLC2A1_01_CpG_15.16
X095_SLC2A1_01_CpG_17
X095_SLC2A1_01_CpG_18
X095_SLC2A1_01_CpG_19.20
X095_SLC2A1_01_CpG_21
X100_SPUVE_02_CpG_3
X100_SPUVE_02_CpG_22
X100_SPUVE_02_CpG_23.24
X100_SPUVE_02_CpG_25
X100_SPUVE_02_CpG_26.27.28
X100_SPUVE_02_CpG_34.35
X100_SPUVE_02_CpG_44
X100_SPUVE_02_CpG_46.47.48.49
X100_SPUVE_02_CpG_50
X100_SPUVE_02_CpG_51.52
X100_SPUVE_02_CpG_53
X100_SPUVE_02_CpG_54
X100_SPUVE_02_CpG_55.56
X100_SPUVE_02_CpG_57.58
X100_SPUVE_02_CpG_59
X100_SPUVE_02_CpG_60
X100_SPUVE_02_CpG_61
X100_SPUVE_02_CpG_62
X100_SPUVE_02_CpG_63.64
X112_UGCGL2_14_CpG_1.2
X112_UGCGL2_14_CpG_3
X112_UGCGL2_14_CpG_4.5
X112_UGCGL2_14_CpG_7.8
X112_UGCGL2_14_CpG_9.10
X112_UGCGL2_14_CpG_11.12
X112_UGCGL2_14_CpG_14.15
X112_UGCGL2_14_CpG_16.17
X112_UGCGL2_14_CpG_18.19
X112_UGCGL2_14_CpG_24.25
X112_UGCGL2_14_CpG_27
X112_UGCGL2_14_CpG_28
X112_UGCGL2_14_CpG_29
X112_UGCGL2_14_CpG_35.36
X112_UGCGL2_14_CpG_37
X112_UGCGL2_14_CpG_38
X112_UGCGL2_14_CpG_39
X112_UGCGL2_14_CpG_46
X112_UGCGL2_14_CpG_47
X112_UGCGL2_14_CpG_48
X112_UGCGL2_14_CpG_49
X112_UGCGL2_14_CpG_50.51
X112_UGCGL2_14_CpG_57
X112_UGCGL2_14_CpG_59
X112_UGCGL2_14_CpG_60.61
X112_UGCGL2_14_CpG_67.68
X112_UGCGL2_14_CpG_74
X112_UGCGL2_14_CpG_75.76
RASSF1_CpG_3.4.5
RASSF1_CpG_6.7.8
RASSF1_CpG_9
RASSF1_CpG_11
RASSF1_CpG_12
RASSF1_CpG_13
RASSF1_CpG_19
RASSF1_CpG_20
RASSF1_CpG_21
RASSF1_CpG_22.23.24.25.26
RASSF1_CpG_27
RASSF1_CpG_28.29
RASSF1_CpG_30
RASSF1_CpG_31.32
RASSF1_CpG_33.34
RASSF1_CpG_35
RASSF1_CpG_36
RASSF1_CpG_37
RASSF1_CpG_42.43.44.45
RASSF1_CpG_46.47
RASSF1_CpG_48
RASSF1_CpG_49.50
RASSF1_CpG_51.52
X041_GYPC_01_CpG_1.2.3
X041_GYPC_01_CpG_4
X041_GYPC_01_CpG_5
X041_GYPC_01_CpG_11
X041_GYPC_01_CpG_12.13
X041_GYPC_01_CpG_14
X041_GYPC_01_CpG_20.21.22
X041_GYPC_01_CpG_23.24
X041_GYPC_01_CpG_25.26.27
X041_GYPC_01_CpG_28
X041_GYPC_01_CpG_29
X030_FGFR1_01_CpG_1.2
X030_FGFR1_01_CpG_3
X030_FGFR1_01_CpG_4.5
X030_FGFR1_01_CpG_10
X030_FGFR1_01_CpG_11.12
X030_FGFR1_01_CpG_13
X030_FGFR1_01_CpG_14.15
X030_FGFR1_01_CpG_16
X030_FGFR1_01_CpG_17
X030_FGFR1_01_CpG_18.19
X049_HOXD13_001_CpG_1
X049_HOXD13_001_CpG_2.3
X049_HOXD13_001_CpG_4.5.6.7
X049_HOXD13_001_CpG_8.9.10.11.12
X049_HOXD13_001_CpG_13.14
X049_HOXD13_001_CpG_15
X049_HOXD13_001_CpG_16
X049_HOXD13_001_CpG_17.18.19
X049_HOXD13_001_CpG_20.21
X049_HOXD13_001_CpG_22
X073_NFKB1_01_CpG_5
X073_NFKB1_01_CpG_64
X073_NFKB1_01_CpG_65.66
X073_NFKB1_01_CpG_76
X099_SNX9_01_CpG_1.2.3.4
X099_SNX9_01_CpG_6
X099_SNX9_01_CpG_7
X099_SNX9_01_CpG_8.9
X099_SNX9_01_CpG_10.11
X099_SNX9_01_CpG_13.14.15.16.17
X099_SNX9_01_CpG_24.25.26.27
X099_SNX9_01_CpG_29.30.31.32
X099_SNX9_01_CpG_33.34
X099_SNX9_01_CpG_35.36.37.38
X099_SNX9_01_CpG_39.40.41
X099_SNX9_01_CpG_42.43.44.45
X099_SNX9_01_CpG_46.47.48.49
X099_SNX9_01_CpG_50
X099_SNX9_01_CpG_51
X099_SNX9_01_CpG_52.53
X099_SNX9_01_CpG_54.55.56.57
X129_CDKN2A_01_CpG_1.2
X129_CDKN2A_01_CpG_3.4
X129_CDKN2A_01_CpG_6
X129_CDKN2A_01_CpG_7
X129_CDKN2A_01_CpG_9
X129_CDKN2A_01_CpG_10
X129_CDKN2A_01_CpG_12
X129_CDKN2A_01_CpG_13
X129_CDKN2A_01_CpG_14.15
X129_CDKN2A_01_CpG_16.17.18
X129_CDKN2A_01_CpG_19
X129_CDKN2A_01_CpG_20
X129_CDKN2A_01_CpG_26
X075_NR2F2_01_CpG_2
X075_NR2F2_01_CpG_3.4
X075_NR2F2_01_CpG_5
X075_NR2F2_01_CpG_6
X075_NR2F2_01_CpG_7.8
X075_NR2F2_01_CpG_10
X075_NR2F2_01_CpG_11
X075_NR2F2_01_CpG_15.16.17.18
X075_NR2F2_01_CpG_21.22
X075_NR2F2_01_CpG_23.24.25
X075_NR2F2_01_CpG_29
X075_NR2F2_01_CpG_31.32
X075_NR2F2_01_CpG_39
X075_NR2F2_01_CpG_40.41
X075_NR2F2_01_CpG_42.43
X075_NR2F2_01_CpG_45
X075_NR2F2_01_CpG_46
X075_NR2F2_01_CpG_47
X075_NR2F2_01_CpG_48
X075_NR2F2_01_CpG_49
X025_EDG1_01_CpG_2.3.4
X025_EDG1_01_CpG_5.6
X025_EDG1_01_CpG_13
X025_EDG1_01_CpG_14
X025_EDG1_01_CpG_17
X025_EDG1_01_CpG_18
X025_EDG1_01_CpG_35
X025_EDG1_01_CpG_37.38
X071_NBL1_01_CpG_2.3.4.5
X071_NBL1_01_CpG_6
X071_NBL1_01_CpG_7
X071_NBL1_01_CpG_8
X071_NBL1_01_CpG_11
X071_NBL1_01_CpG_12.13
X071_NBL1_01_CpG_15
X071_NBL1_01_CpG_16
X071_NBL1_01_CpG_26.27.28
X071_NBL1_01_CpG_29.30.31
X071_NBL1_01_CpG_32.33.34
X085_RGS16_01_CpG_1
X085_RGS16_01_CpG_2
X085_RGS16_01_CpG_3.4
X085_RGS16_01_CpG_6.7.8
X085_RGS16_01_CpG_9.10
X085_RGS16_01_CpG_11
X085_RGS16_01_CpG_12
X085_RGS16_01_CpG_13.14
X085_RGS16_01_CpG_15
X085_RGS16_01_CpG_16.17
X085_RGS16_01_CpG_18
X085_RGS16_01_CpG_19
X085_RGS16_01_CpG_20
X085_RGS16_01_CpG_21.22
X085_RGS16_01_CpG_23
X085_RGS16_01_CpG_24.25
X085_RGS16_01_CpG_26.27
X085_RGS16_01_CpG_28
X085_RGS16_01_CpG_29.30.31
X085_RGS16_01_CpG_32
X085_RGS16_01_CpG_33
X085_RGS16_01_CpG_34
X085_RGS16_01_CpG_35
X085_RGS16_01_CpG_37.38
X085_RGS16_01_CpG_39
X085_RGS16_01_CpG_40
X093_SERPINA5_01_CpG_1
X093_SERPINA5_01_CpG_2
X093_SERPINA5_01_CpG_5.6
X093_SERPINA5_01_CpG_22
X093_SERPINA5_01_CpG_27.28.29
X093_SERPINA5_01_CpG_41.42
X093_SERPINA5_01_CpG_43
X093_SERPINA5_01_CpG_48.49
X093_SERPINA5_01_CpG_56.57
X093_SERPINA5_01_CpG_58.59.60.61.62.63.64
X093_SERPINA5_01_CpG_65
X146_MEIS1_02_CpG_1
X146_MEIS1_02_CpG_2
X146_MEIS1_02_CpG_3.4
X146_MEIS1_02_CpG_5
X146_MEIS1_02_CpG_6
X146_MEIS1_02_CpG_7.8
X146_MEIS1_02_CpG_9
X146_MEIS1_02_CpG_10.11
X146_MEIS1_02_CpG_12
X146_MEIS1_02_CpG_16
X146_MEIS1_02_CpG_18
X146_MEIS1_02_CpG_29.30
X146_MEIS1_02_CpG_31
X146_MEIS1_02_CpG_32.33
X146_MEIS1_02_CpG_40
X146_MEIS1_02_CpG_47
X146_MEIS1_02_CpG_48.49
X146_MEIS1_02_CpG_50.51
X146_MEIS1_02_CpG_52
X146_MEIS1_02_CpG_53.54
X155_RBP1_57_CpG_1.2
X155_RBP1_57_CpG_3.4.5
X155_RBP1_57_CpG_6
X155_RBP1_57_CpG_7.8.9.10
X155_RBP1_57_CpG_11.12
X155_RBP1_57_CpG_13.14
X155_RBP1_57_CpG_15.16.17
X155_RBP1_57_CpG_18
X155_RBP1_57_CpG_19
X155_RBP1_57_CpG_20
X155_RBP1_57_CpG_22.23
X155_RBP1_57_CpG_24
X155_RBP1_57_CpG_25
X155_RBP1_57_CpG_26
X155_RBP1_57_CpG_27
X011_AZGP1_01_CpG_1.2
X011_AZGP1_01_CpG_3
X011_AZGP1_01_CpG_4
X011_AZGP1_01_CpG_6.7
X011_AZGP1_01_CpG_8.9.10.11
X011_AZGP1_01_CpG_12
X011_AZGP1_01_CpG_13.14
X013_BCL11A_01_01_CpG_1.2
X013_BCL11A_01_01_CpG_3
X013_BCL11A_01_01_CpG_4
X013_BCL11A_01_01_CpG_5
X013_BCL11A_01_01_CpG_7
X013_BCL11A_01_01_CpG_8.9
X013_BCL11A_01_01_CpG_11
X013_BCL11A_01_01_CpG_12
X013_BCL11A_01_01_CpG_13.14
X013_BCL11A_01_01_CpG_15
X013_BCL11A_01_01_CpG_16.17.18.19
X013_BCL11A_01_01_CpG_20
X013_BCL11A_01_01_CpG_21
X035_FN14_01_CpG_1.2.3.4.5.6.7
X035_FN14_01_CpG_9.10
X035_FN14_01_CpG_12.13
X035_FN14_01_CpG_25.26.27
X035_FN14_01_CpG_28
X035_FN14_01_CpG_39
X035_FN14_01_CpG_40.41
X035_FN14_01_CpG_42.43
X035_FN14_01_CpG_44.45.46
X035_FN14_01_CpG_48.49
X035_FN14_01_CpG_50
X040_GUCY1A3_01_CpG_1
X040_GUCY1A3_01_CpG_2.3
X040_GUCY1A3_01_CpG_4.5.6.7
X040_GUCY1A3_01_CpG_8.9
X040_GUCY1A3_01_CpG_11
X040_GUCY1A3_01_CpG_12
X040_GUCY1A3_01_CpG_13
X040_GUCY1A3_01_CpG_15.16.17
X040_GUCY1A3_01_CpG_18
X040_GUCY1A3_01_CpG_19
X040_GUCY1A3_01_CpG_20
X040_GUCY1A3_01_CpG_21.22.23.24
X040_GUCY1A3_01_CpG_25
X047_ID3_01_CpG_1
X047_ID3_01_CpG_3
X047_ID3_01_CpG_4.5
X047_ID3_01_CpG_6
X047_ID3_01_CpG_7
X047_ID3_01_CpG_8
X047_ID3_01_CpG_9
X047_ID3_01_CpG_10
X047_ID3_01_CpG_11
X047_ID3_01_CpG_16.17
X047_ID3_01_CpG_18.19
X047_ID3_01_CpG_20.21
X047_ID3_01_CpG_22
X047_ID3_01_CpG_23
X058_LGMN_01_CpG_1.2.3.4
X058_LGMN_01_CpG_5.6.7
X058_LGMN_01_CpG_8
X058_LGMN_01_CpG_18
X058_LGMN_01_CpG_19.20
X058_LGMN_01_CpG_21
X058_LGMN_01_CpG_23
X058_LGMN_01_CpG_24
X058_LGMN_01_CpG_25
X058_LGMN_01_CpG_29
X058_LGMN_01_CpG_31.32
X058_LGMN_01_CpG_37
X058_LGMN_01_CpG_38.39
X058_LGMN_01_CpG_40.41
X058_LGMN_01_CpG_42.43
X058_LGMN_01_CpG_44
X058_LGMN_01_CpG_45
X058_LGMN_01_CpG_49
X058_LGMN_01_CpG_50
X058_LGMN_01_CpG_51.52
X058_LGMN_01_CpG_57
X067_MIG2_01_CpG_1
X067_MIG2_01_CpG_2
X067_MIG2_01_CpG_3.4
X067_MIG2_01_CpG_5
X080_PLCG1_01_CpG_1.2
X080_PLCG1_01_CpG_4.5.6
X080_PLCG1_01_CpG_8
X080_PLCG1_01_CpG_14.15
X080_PLCG1_01_CpG_16.17.18
X080_PLCG1_01_CpG_20
X080_PLCG1_01_CpG_26
X080_PLCG1_01_CpG_27.28
X080_PLCG1_01_CpG_29
X080_PLCG1_01_CpG_30.31
X080_PLCG1_01_CpG_32
X080_PLCG1_01_CpG_33
X080_PLCG1_01_CpG_34.35.36.37
X087_S100P_01_CpG_1
X087_S100P_01_CpG_2
X087_S100P_01_CpG_3.4
X087_S100P_01_CpG_5
X087_S100P_01_CpG_8.9.10
X087_S100P_01_CpG_11.12.13.14.15
X087_S100P_01_CpG_16
X087_S100P_01_CpG_17
X087_S100P_01_CpG_18
X087_S100P_01_CpG_23
X087_S100P_01_CpG_24
X087_S100P_01_CpG_25
X087_S100P_01_CpG_26.27
X087_S100P_01_CpG_28
X087_S100P_01_CpG_29.30.31
X087_S100P_01_CpG_32
X087_S100P_01_CpG_34.35.36
X087_S100P_01_CpG_37.38.39.40
X087_S100P_01_CpG_41.42.43
X087_S100P_01_CpG_44.45
X087_S100P_01_CpG_46.47
X087_S100P_01_CpG_49.50
X087_S100P_01_CpG_51.52
X087_S100P_01_CpG_53
X087_S100P_01_CpG_54
X087_S100P_01_CpG_55.56.57.58.59.60.61
X087_S100P_01_CpG_62.63
X087_S100P_01_CpG_64.65.66.67
X087_S100P_01_CpG_68.69
X087_S100P_01_CpG_70
X087_S100P_01_CpG_71
X098_SMG1_01_CpG_1
X098_SMG1_01_CpG_3
X098_SMG1_01_CpG_4.5
X098_SMG1_01_CpG_12.13
X098_SMG1_01_CpG_14.15.16.17
X098_SMG1_01_CpG_18.19
X098_SMG1_01_CpG_20.21
X098_SMG1_01_CpG_22.23.24
X098_SMG1_01_CpG_26
X103_TACSTD2_01_CpG_1.2
X103_TACSTD2_01_CpG_4
X103_TACSTD2_01_CpG_5
X103_TACSTD2_01_CpG_6
X103_TACSTD2_01_CpG_7.8
X103_TACSTD2_01_CpG_9
X103_TACSTD2_01_CpG_10.11.12.13.14.15
X103_TACSTD2_01_CpG_17.18
X103_TACSTD2_01_CpG_20
X103_TACSTD2_01_CpG_21.22.23
X103_TACSTD2_01_CpG_24.25.26
X103_TACSTD2_01_CpG_27.28
X103_TACSTD2_01_CpG_29.30
X103_TACSTD2_01_CpG_36.37
X103_TACSTD2_01_CpG_38.39.40
X103_TACSTD2_01_CpG_49.50
X103_TACSTD2_01_CpG_57.58
X103_TACSTD2_01_CpG_59
X103_TACSTD2_01_CpG_60.61
X103_TACSTD2_01_CpG_62.63.64.65
X103_TACSTD2_01_CpG_66
X111_UGCG_13_CpG_1
X111_UGCG_13_CpG_2
X111_UGCG_13_CpG_3
X111_UGCG_13_CpG_4.5
X111_UGCG_13_CpG_6
X111_UGCG_13_CpG_7.8
X111_UGCG_13_CpG_9.10
X111_UGCG_13_CpG_11
X111_UGCG_13_CpG_15.16.17.18
X111_UGCG_13_CpG_19
X111_UGCG_13_CpG_20
X117_PSCB5_02_19_CpG_1.2.3
X117_PSCB5_02_19_CpG_6
X117_PSCB5_02_19_CpG_8.9
X117_PSCB5_02_19_CpG_10.11
X117_PSCB5_02_19_CpG_12
X117_PSCB5_02_19_CpG_13.14.15
X117_PSCB5_02_19_CpG_16
X131_CEBPA_33_CpG_1
X131_CEBPA_33_CpG_2.3.4
X131_CEBPA_33_CpG_5.6.7.8.9
X131_CEBPA_33_CpG_10.11
X131_CEBPA_33_CpG_12.13
X131_CEBPA_33_CpG_14
X131_CEBPA_33_CpG_15.16
X131_CEBPA_33_CpG_18.19.20
X131_CEBPA_33_CpG_21
X131_CEBPA_33_CpG_22
X131_CEBPA_33_CpG_24
X131_CEBPA_33_CpG_25
X131_CEBPA_33_CpG_26.27
X131_CEBPA_33_CpG_28
X131_CEBPA_33_CpG_29
X131_CEBPA_33_CpG_32
X131_CEBPA_33_CpG_33.34
X131_CEBPA_33_CpG_36.37
X131_CEBPA_33_CpG_38
X131_CEBPA_33_CpG_40.41.42
X131_CEBPA_33_CpG_43
X131_CEBPA_33_CpG_45
X142_GNG2_01_CpG_1
X142_GNG2_01_CpG_2
X142_GNG2_01_CpG_3
X142_GNG2_01_CpG_4
X142_GNG2_01_CpG_5
X142_GNG2_01_CpG_6
X142_GNG2_01_CpG_7
X142_GNG2_01_CpG_8.9.10.11.12
X162_TNFRSF12A_64_CpG_1.2.3.4.5.6.7
X162_TNFRSF12A_64_CpG_9.10
X162_TNFRSF12A_64_CpG_12.13
X162_TNFRSF12A_64_CpG_25.26.27
X162_TNFRSF12A_64_CpG_28
X162_TNFRSF12A_64_CpG_39
X162_TNFRSF12A_64_CpG_40.41
X162_TNFRSF12A_64_CpG_42.43
X162_TNFRSF12A_64_CpG_44.45.46
X162_TNFRSF12A_64_CpG_48.49
X162_TNFRSF12A_64_CpG_50
HOXA4_SQ02_CpG_1
HOXA4_SQ02_CpG_2.3
HOXA4_SQ02_CpG_5.6.7.8
HOXA4_SQ02_CpG_13.14.15.16
HOXA4_SQ02_CpG_17.18.19
HOXA4_SQ02_CpG_22.23
HOXA4_SQ02_CpG_36.37.38
HOXA4_SQ02_CpG_39.40
HOXA4_SQ02_CpG_41.42
ESR1_01_01_CpG_1
ESR1_01_01_CpG_2
ESR1_01_01_CpG_3.4
ESR1_01_01_CpG_5.6.7.8
ESR1_01_01_CpG_10
ESR1_01_01_CpG_11
ESR1_01_01_CpG_12
ESR1_01_01_CpG_13.14.15.16
ESR1_01_01_CpG_17.18.19
ESR1_01_01_CpG_20
ESR1_01_01_CpG_21.22
ESR1_01_01_CpG_23
ESR1_01_01_CpG_24
ESR1_01_01_CpG_25.26
p53_CpG_10
p53_CpG_11.12
p53_CpG_15.16
p53_CpG_17
p53_CpG_19.20
p16_01_CpG_1.2
p16_01_CpG_3.4
p16_01_CpG_6
p16_01_CpG_7
p16_01_CpG_9
p16_01_CpG_10
p16_01_CpG_11
p16_01_CpG_12
p16_01_CpG_13
X006_HOXD11_001_CpG_21
X006_HOXD11_001_CpG_31
X006_HOXD11_001_CpG_42
seq.GSTP1.300.100_01_CpG_8
seq.GSTP1.300.100_01_CpG_9
seq.GSTP1.300.100_01_CpG_10.11.12
seq.GSTP1.300.100_01_CpG_13
seq.GSTP1.300.100_01_CpG_24.25
seq.GSTP1.300.100_01_CpG_26.27.28.29
seq.GSTP1.300.100_01_CpG_30
seq.GSTP1.300.100_01_CpG_31.32
seq.GSTP1.300.100_01_CpG_33
seq.GSTP1.300.100_01_CpG_34.35
seq.GSTP1.300.100_01_CpG_36
seq.GSTP1.300.100_01_CpG_37.38.39
seq.GSTP1.300.100_01_CpG_40
X016_ACTG1.06_CpG_2.3.4
X016_ACTG1.06_CpG_5
X016_ACTG1.06_CpG_6
X016_ACTG1.06_CpG_7.8.9.10
X016_ACTG1.06_CpG_11.12.13.14
X016_ACTG1.06_CpG_15.16
X016_ACTG1.06_CpG_17
X016_ACTG1.06_CpG_18
X016_ACTG1.06_CpG_19
X016_ACTG1.06_CpG_20.21.22.23.24
X016_ACTG1.06_CpG_26
X032_ACTG1.02_CpG_8
X032_ACTG1.02_CpG_59
X032_ACTG1.02_CpG_60.61
X669_Notch4_01_CpG_6
X669_Notch4_01_CpG_7
X669_Notch4_01_CpG_9.10
X669_Notch4_01_CpG_16
X669_Notch4_01_CpG_24
X669_Notch4_01_CpG_25.26
HOXA10_SQ02_CpG_27
HOXA10_SQ02_CpG_34
HOXA10_SQ02_CpG_39.40
X057_LCN2_01_CpG_1.2
X057_LCN2_01_CpG_3
X057_LCN2_01_CpG_6
X057_LCN2_01_CpG_7.8.9
X057_LCN2_01_CpG_11.12
X057_LCN2_01_CpG_13.14.15
X057_LCN2_01_CpG_16
X057_LCN2_01_CpG_19
X057_LCN2_01_CpG_20
X057_LCN2_01_CpG_23
X057_LCN2_01_CpG_24
X057_LCN2_01_CpG_26
X057_LCN2_01_CpG_27
X057_LCN2_01_CpG_31.32.33.34
X057_LCN2_01_CpG_35
X078_PHEMX_01_CpG_15
X078_PHEMX_01_CpG_21.22
X078_PHEMX_01_CpG_26
X078_PHEMX_01_CpG_27
X078_PHEMX_01_CpG_28
X078_PHEMX_01_CpG_29
X078_PHEMX_01_CpG_30
X078_PHEMX_01_CpG_31
X078_PHEMX_01_CpG_32
X078_PHEMX_01_CpG_39
X088_SCAP2_01_CpG_2.3
X088_SCAP2_01_CpG_4
X088_SCAP2_01_CpG_6.7.8
X088_SCAP2_01_CpG_9.10
X088_SCAP2_01_CpG_11
X088_SCAP2_01_CpG_13
X088_SCAP2_01_CpG_14.15
X088_SCAP2_01_CpG_17
X088_SCAP2_01_CpG_18
X088_SCAP2_01_CpG_19.20
X088_SCAP2_01_CpG_22
X088_SCAP2_01_CpG_23.24
X088_SCAP2_01_CpG_25
X088_SCAP2_01_CpG_26
X088_SCAP2_01_CpG_27.28
X138_EVI1_40_CpG_1
X138_EVI1_40_CpG_8
X138_EVI1_40_CpG_9
X138_EVI1_40_CpG_14
X138_EVI1_40_CpG_15
X138_EVI1_40_CpG_16.17
X138_EVI1_40_CpG_21.22
X138_EVI1_40_CpG_28.29
X138_EVI1_40_CpG_30
X152_PITX2_54_CpG_2
X152_PITX2_54_CpG_3
X152_PITX2_54_CpG_5
X152_PITX2_54_CpG_6.7
X152_PITX2_54_CpG_8.9
X152_PITX2_54_CpG_10
X152_PITX2_54_CpG_12.13
X152_PITX2_54_CpG_14
X152_PITX2_54_CpG_15.16
X152_PITX2_54_CpG_17
X152_PITX2_54_CpG_24.25
X152_PITX2_54_CpG_26.27
X152_PITX2_54_CpG_29.30
X152_PITX2_54_CpG_31.32
X152_PITX2_54_CpG_34
X152_PITX2_54_CpG_36
X009_APOC1_01_01_CpG_1
X009_APOC1_01_01_CpG_2
X009_APOC1_01_01_CpG_3
X009_APOC1_01_01_CpG_4
X009_APOC1_01_01_CpG_5
X009_APOC1_01_01_CpG_6
X009_APOC1_01_01_CpG_7
X009_APOC1_01_01_CpG_8.9
X009_APOC1_01_01_CpG_11.12
X009_APOC1_01_01_CpG_14
X009_APOC1_01_01_CpG_18
X009_APOC1_01_01_CpG_19
X009_APOC1_01_01_CpG_20
X012_BAI2_01_CpG_2
X012_BAI2_01_CpG_4
X012_BAI2_01_CpG_5.6.7
X012_BAI2_01_CpG_8
X012_BAI2_01_CpG_9
X012_BAI2_01_CpG_10
X012_BAI2_01_CpG_11
X012_BAI2_01_CpG_12.13
X012_BAI2_01_CpG_14
X012_BAI2_01_CpG_15
X012_BAI2_01_CpG_16
X012_BAI2_01_CpG_22.23
X012_BAI2_01_CpG_24.25.26.27.28
X012_BAI2_01_CpG_29.30
X012_BAI2_01_CpG_31
X012_BAI2_01_CpG_32
X012_BAI2_01_CpG_33
X012_BAI2_01_CpG_34.35
X012_BAI2_01_CpG_36
X012_BAI2_01_CpG_37
X012_BAI2_01_CpG_39
X012_BAI2_01_CpG_40
X012_BAI2_01_CpG_41.42
X012_BAI2_01_CpG_43
X012_BAI2_01_CpG_44
X014_BCL11A_02_01_CpG_1
X014_BCL11A_02_01_CpG_2
X014_BCL11A_02_01_CpG_3.4
X014_BCL11A_02_01_CpG_5
X014_BCL11A_02_01_CpG_6
X014_BCL11A_02_01_CpG_7.8
X014_BCL11A_02_01_CpG_9.10
X014_BCL11A_02_01_CpG_11
X014_BCL11A_02_01_CpG_12.13.14
X014_BCL11A_02_01_CpG_15
X014_BCL11A_02_01_CpG_16.17
X014_BCL11A_02_01_CpG_18.19
X014_BCL11A_02_01_CpG_20
X014_BCL11A_02_01_CpG_21
X014_BCL11A_02_01_CpG_22
X014_BCL11A_02_01_CpG_23.24
X014_BCL11A_02_01_CpG_26.27
X014_BCL11A_02_01_CpG_28
X014_BCL11A_02_01_CpG_29
X014_BCL11A_02_01_CpG_32
X014_BCL11A_02_01_CpG_33.34
X014_BCL11A_02_01_CpG_36
X014_BCL11A_02_01_CpG_37
X014_BCL11A_02_01_CpG_38.39
X014_BCL11A_02_01_CpG_40
X014_BCL11A_02_01_CpG_41.42.43.44
X014_BCL11A_02_01_CpG_45
X014_BCL11A_02_01_CpG_46
X020_CTNNAL1_01_CpG_1
X020_CTNNAL1_01_CpG_3
X020_CTNNAL1_01_CpG_4
X020_CTNNAL1_01_CpG_5
X020_CTNNAL1_01_CpG_7.8
X020_CTNNAL1_01_CpG_9
X020_CTNNAL1_01_CpG_10.11
X020_CTNNAL1_01_CpG_12
X020_CTNNAL1_01_CpG_13
X020_CTNNAL1_01_CpG_14.15
X020_CTNNAL1_01_CpG_16
X020_CTNNAL1_01_CpG_17
X020_CTNNAL1_01_CpG_18
X020_CTNNAL1_01_CpG_23.24.25
X020_CTNNAL1_01_CpG_26.27.28
X020_CTNNAL1_01_CpG_29
X020_CTNNAL1_01_CpG_30
X020_CTNNAL1_01_CpG_31
X027_EMR1_01_CpG_2
X027_EMR1_01_CpG_3
X027_EMR1_01_CpG_4
X027_EMR1_01_CpG_5
X027_EMR1_01_CpG_7.8
X027_EMR1_01_CpG_9
X027_EMR1_01_CpG_10.11
X027_EMR1_01_CpG_12
X027_EMR1_01_CpG_13
X027_EMR1_01_CpG_14.15
X027_EMR1_01_CpG_21
X032_FLJ21820_01_CpG_1
X032_FLJ21820_01_CpG_2
X032_FLJ21820_01_CpG_3.4
X032_FLJ21820_01_CpG_5
X032_FLJ21820_01_CpG_7
X032_FLJ21820_01_CpG_8
X032_FLJ21820_01_CpG_10
X032_FLJ21820_01_CpG_11
X032_FLJ21820_01_CpG_12.13.14
X032_FLJ21820_01_CpG_15
X032_FLJ21820_01_CpG_16
X032_FLJ21820_01_CpG_17.18.19.20
X036_FOXO1A_3p_01_CpG_2.3
X036_FOXO1A_3p_01_CpG_4
X036_FOXO1A_3p_01_CpG_5
X036_FOXO1A_3p_01_CpG_6
X036_FOXO1A_3p_01_CpG_7.8
X036_FOXO1A_3p_01_CpG_9
X036_FOXO1A_3p_01_CpG_13
X036_FOXO1A_3p_01_CpG_15
X045_HOXB2_01_CpG_1
X045_HOXB2_01_CpG_2.3.4
X045_HOXB2_01_CpG_5
X045_HOXB2_01_CpG_6.7
X045_HOXB2_01_CpG_8
X045_HOXB2_01_CpG_9
X045_HOXB2_01_CpG_10.11.12.13
X045_HOXB2_01_CpG_18.19.20
X045_HOXB2_01_CpG_23.24.25
X045_HOXB2_01_CpG_26
X045_HOXB2_01_CpG_27.28
X045_HOXB2_01_CpG_29.30.31.32
X045_HOXB2_01_CpG_39
X045_HOXB2_01_CpG_40
X045_HOXB2_01_CpG_46.47
X051_KIAA0476_01_CpG_1
X051_KIAA0476_01_CpG_2
X051_KIAA0476_01_CpG_3
X051_KIAA0476_01_CpG_4
X051_KIAA0476_01_CpG_5
X051_KIAA0476_01_CpG_6.7.8.9
X051_KIAA0476_01_CpG_11
X051_KIAA0476_01_CpG_12.13.14
X051_KIAA0476_01_CpG_15.16.17.18.19
X051_KIAA0476_01_CpG_22.23.24.25.26
X051_KIAA0476_01_CpG_28.29
X051_KIAA0476_01_CpG_30
X051_KIAA0476_01_CpG_32
X051_KIAA0476_01_CpG_33.34
X051_KIAA0476_01_CpG_35
X060_LOC55971_01_CpG_1
X060_LOC55971_01_CpG_2
X060_LOC55971_01_CpG_3
X060_LOC55971_01_CpG_4
X060_LOC55971_01_CpG_5.6
X060_LOC55971_01_CpG_7.8.9.10
X060_LOC55971_01_CpG_11.12
X060_LOC55971_01_CpG_13.14.15.16.17
X060_LOC55971_01_CpG_18
X060_LOC55971_01_CpG_19
X060_LOC55971_01_CpG_20.21.22
X060_LOC55971_01_CpG_23.24.25.26
X060_LOC55971_01_CpG_27.28.29
X060_LOC55971_01_CpG_30.31.32.33.34.35
X060_LOC55971_01_CpG_36
X060_LOC55971_01_CpG_37.38.39
X060_LOC55971_01_CpG_40.41
X060_LOC55971_01_CpG_42.43
X060_LOC55971_01_CpG_44.45.46
X060_LOC55971_01_CpG_47.48
X060_LOC55971_01_CpG_49.50.51
X060_LOC55971_01_CpG_52
X060_LOC55971_01_CpG_53.54
X060_LOC55971_01_CpG_55
X060_LOC55971_01_CpG_60
X074_NFKBIB_01_CpG_2
X074_NFKBIB_01_CpG_4
X074_NFKBIB_01_CpG_6
X074_NFKBIB_01_CpG_7
X074_NFKBIB_01_CpG_8.9
X074_NFKBIB_01_CpG_10
X074_NFKBIB_01_CpG_11.12.13.14
X074_NFKBIB_01_CpG_15
X074_NFKBIB_01_CpG_16
X074_NFKBIB_01_CpG_17
X074_NFKBIB_01_CpG_18.19
X074_NFKBIB_01_CpG_21.22
X074_NFKBIB_01_CpG_23
X074_NFKBIB_01_CpG_26
X074_NFKBIB_01_CpG_28
X074_NFKBIB_01_CpG_29.30
X074_NFKBIB_01_CpG_35.36.37
X074_NFKBIB_01_CpG_40.41
X074_NFKBIB_01_CpG_42.43
X074_NFKBIB_01_CpG_44
X074_NFKBIB_01_CpG_45
X074_NFKBIB_01_CpG_47
X109_TUBB_11_CpG_1
X109_TUBB_11_CpG_2
X109_TUBB_11_CpG_4.5
X109_TUBB_11_CpG_6
X109_TUBB_11_CpG_7
X109_TUBB_11_CpG_8
X109_TUBB_11_CpG_9.10
X109_TUBB_11_CpG_11
X109_TUBB_11_CpG_12.13
X109_TUBB_11_CpG_15
X113_VIL2_15_CpG_1.2
X113_VIL2_15_CpG_3.4
X113_VIL2_15_CpG_5.6
X113_VIL2_15_CpG_10.11.12
X113_VIL2_15_CpG_25.26
X113_VIL2_15_CpG_27.28
X113_VIL2_15_CpG_33
X113_VIL2_15_CpG_34.35
X113_VIL2_15_CpG_36.37.38
X113_VIL2_15_CpG_39
X113_VIL2_15_CpG_40.41
X113_VIL2_15_CpG_52.53.54.55
X113_VIL2_15_CpG_56.57
X113_VIL2_15_CpG_58
X113_VIL2_15_CpG_59.60
X113_VIL2_15_CpG_61
X113_VIL2_15_CpG_65
X113_VIL2_15_CpG_66.67
X113_VIL2_15_CpG_68
X119_ABCB1_21_CpG_1.2
X119_ABCB1_21_CpG_4
X119_ABCB1_21_CpG_5
X119_ABCB1_21_CpG_6.7
X119_ABCB1_21_CpG_8.9.10
X119_ABCB1_21_CpG_11.12
X119_ABCB1_21_CpG_14
X119_ABCB1_21_CpG_15.16
X119_ABCB1_21_CpG_18.19
X136_DPEP2_38_CpG_1.2
X136_DPEP2_38_CpG_6
X136_DPEP2_38_CpG_7
X136_DPEP2_38_CpG_8.9
X136_DPEP2_38_CpG_11.12
X136_DPEP2_38_CpG_13
X136_DPEP2_38_CpG_14.15
X136_DPEP2_38_CpG_16
X136_DPEP2_38_CpG_17
X136_DPEP2_38_CpG_18
X136_DPEP2_38_CpG_19.20
X136_DPEP2_38_CpG_21.22.23.24
X136_DPEP2_38_CpG_25
X136_DPEP2_38_CpG_26.27
X136_DPEP2_38_CpG_28
X153_PLEKHC1_55_CpG_1
X153_PLEKHC1_55_CpG_2
X153_PLEKHC1_55_CpG_3.4
X153_PLEKHC1_55_CpG_5
HOXA1_SQ05_CpG_1.2
HOXA1_SQ05_CpG_4.5
HOXA1_SQ05_CpG_8.9
HOXA1_SQ05_CpG_10
HOXA1_SQ05_CpG_11
HOXA1_SQ05_CpG_13
HOXA1_SQ05_CpG_14.15
HOXA1_SQ05_CpG_16.17
HOXA1_SQ05_CpG_18.19.20
HOXA1_SQ05_CpG_21
HOXA1_SQ05_CpG_22.23
HOXA1_SQ05_CpG_24
HOXA5_SQ03_CpG_3
HOXA5_SQ03_CpG_4
HOXA5_SQ03_CpG_6
HOXA5_SQ03_CpG_17.18
HOXA5_SQ03_CpG_22
HOXA5_SQ03_CpG_24
HOXA5_SQ03_CpG_25
HOXA5_SQ03_CpG_26.27
HOXA5_SQ03_CpG_28
HOXA5_SQ03_CpG_29
HOXA5_SQ03_CpG_30
HOXA5_SQ03_CpG_31.32.33.34.35
HOXA5_SQ03_CpG_36
HOXA5_SQ03_CpG_38
HOXA5_SQ03_CpG_39
MGMT_01_03_CpG_5.6
MGMT_01_03_CpG_7
MGMT_01_03_CpG_8
MGMT_01_03_CpG_9.10.11
MGMT_01_03_CpG_18.19.20.21
MGMT_01_03_CpG_22.23
MGMT_01_03_CpG_24.25.26
X001_CDKN2A_01_02_CpG_1.2
X001_CDKN2A_01_02_CpG_3.4
X001_CDKN2A_01_02_CpG_6
X001_CDKN2A_01_02_CpG_7
X001_CDKN2A_01_02_CpG_9
X001_CDKN2A_01_02_CpG_10
X001_CDKN2A_01_02_CpG_11
X001_CDKN2A_01_02_CpG_12
X001_CDKN2A_01_02_CpG_13
X001_CDKN2A_01_02_CpG_14.15
X001_CDKN2A_01_02_CpG_16.17.18
X001_CDKN2A_01_02_CpG_19
X001_CDKN2A_01_02_CpG_20
X001_CDKN2A_01_02_CpG_26
X007_RAR.beta_01_CpG_2
X007_RAR.beta_01_CpG_7.8
X007_RAR.beta_01_CpG_9
X007_RAR.beta_01_CpG_11
X007_RAR.beta_01_CpG_12.13
X007_RAR.beta_01_CpG_14
X007_RAR.beta_01_CpG_15
HOXA5_ABO1_CpG_12.13
HOXA5_ABO1_CpG_17
HOXA5_ABO1_CpG_20
HOXA5_ABO1_CpG_21.22
HOXA5_ABO1_CpG_23
X002_ACTG1.15_CpG_17.18
X018_ACTG1.06_CpG_2
X018_ACTG1.06_CpG_4
X018_ACTG1.06_CpG_5.6
X018_ACTG1.06_CpG_8
X018_ACTG1.06_CpG_9.10
X018_ACTG1.06_CpG_12
X018_ACTG1.06_CpG_13.14.15
X018_ACTG1.06_CpG_16
X018_ACTG1.06_CpG_17.18
X018_ACTG1.06_CpG_19
X039_GAS7_001_CpG_27.28.29.30.31
X039_GAS7_001_CpG_47.48
X039_GAS7_001_CpG_60
X034_FLT3_01_CpG_1.2
X034_FLT3_01_CpG_4
X034_FLT3_01_CpG_5
X034_FLT3_01_CpG_7
X034_FLT3_01_CpG_10.11
X034_FLT3_01_CpG_12
X034_FLT3_01_CpG_14.15.16.17.18
X034_FLT3_01_CpG_23.24
X034_FLT3_01_CpG_25.26.27.28
X034_FLT3_01_CpG_32
X034_FLT3_01_CpG_33
X034_FLT3_01_CpG_34.35
X034_FLT3_01_CpG_36
X034_FLT3_01_CpG_37
X034_FLT3_01_CpG_38.39
X034_FLT3_01_CpG_40
X034_FLT3_01_CpG_41
X034_FLT3_01_CpG_44.45
X050_ISG20_01_CpG_1
X050_ISG20_01_CpG_2
X050_ISG20_01_CpG_4
X050_ISG20_01_CpG_5.6
X050_ISG20_01_CpG_8
X050_ISG20_01_CpG_12
X050_ISG20_01_CpG_13.14.15
X050_ISG20_01_CpG_17
X050_ISG20_01_CpG_19.20
X050_ISG20_01_CpG_21.22
X050_ISG20_01_CpG_23
X050_ISG20_01_CpG_24
X050_ISG20_01_CpG_25
X050_ISG20_01_CpG_26.27
X069_MSLN_01_CpG_1
X069_MSLN_01_CpG_5
X069_MSLN_01_CpG_6
X069_MSLN_01_CpG_7
X069_MSLN_01_CpG_13
X069_MSLN_01_CpG_17
X069_MSLN_01_CpG_18.19.20
X069_MSLN_01_CpG_21
X069_MSLN_01_CpG_23
X069_MSLN_01_CpG_24.25
X069_MSLN_01_CpG_26
X069_MSLN_01_CpG_27
X083_PRO2730_01_CpG_2.3.4
X083_PRO2730_01_CpG_5
X083_PRO2730_01_CpG_6.7.8
X083_PRO2730_01_CpG_10
X083_PRO2730_01_CpG_11.12
X083_PRO2730_01_CpG_17.18
X083_PRO2730_01_CpG_21.22.23
X092_SEMA3F_01_CpG_1
X092_SEMA3F_01_CpG_2
X092_SEMA3F_01_CpG_3
X092_SEMA3F_01_CpG_4
X092_SEMA3F_01_CpG_6.7
X092_SEMA3F_01_CpG_8.9
X092_SEMA3F_01_CpG_10
X092_SEMA3F_01_CpG_11
X092_SEMA3F_01_CpG_12
X092_SEMA3F_01_CpG_13.14.15
X092_SEMA3F_01_CpG_17
X092_SEMA3F_01_CpG_18.19.20.21
X092_SEMA3F_01_CpG_22.23
X092_SEMA3F_01_CpG_24
X092_SEMA3F_01_CpG_27
X092_SEMA3F_01_CpG_28
X092_SEMA3F_01_CpG_29.30
X092_SEMA3F_01_CpG_31
X010_APOC1_02_01_CpG_3
X010_APOC1_02_01_CpG_4
X010_APOC1_02_01_CpG_8
X010_APOC1_02_01_CpG_11.12
X010_APOC1_02_01_CpG_13.14
X010_APOC1_02_01_CpG_15
X010_APOC1_02_01_CpG_16.17
X010_APOC1_02_01_CpG_21.22.23
X010_APOC1_02_01_CpG_24.25.26
X010_APOC1_02_01_CpG_27
X010_APOC1_02_01_CpG_32
X010_APOC1_02_01_CpG_33
X010_APOC1_02_01_CpG_34.35.36
X010_APOC1_02_01_CpG_37
X010_APOC1_02_01_CpG_38
X010_APOC1_02_01_CpG_39.40.41
X010_APOC1_02_01_CpG_42.43
X010_APOC1_02_01_CpG_44.45
X010_APOC1_02_01_CpG_46
X010_APOC1_02_01_CpG_47.48
X010_APOC1_02_01_CpG_49
X016_CDC42EP4_01_CpG_1
X016_CDC42EP4_01_CpG_2.3
X016_CDC42EP4_01_CpG_4
X016_CDC42EP4_01_CpG_5.6.7
X016_CDC42EP4_01_CpG_8.9
X016_CDC42EP4_01_CpG_10
X016_CDC42EP4_01_CpG_18
X021_D2S448_01_CpG_1
X021_D2S448_01_CpG_2
X021_D2S448_01_CpG_4
X021_D2S448_01_CpG_5
X021_D2S448_01_CpG_6
X021_D2S448_01_CpG_10
X021_D2S448_01_CpG_11
X021_D2S448_01_CpG_14
X021_D2S448_01_CpG_15.16
X021_D2S448_01_CpG_17.18
X021_D2S448_01_CpG_19
X021_D2S448_01_CpG_20
X021_D2S448_01_CpG_22
X021_D2S448_01_CpG_24
X021_D2S448_01_CpG_25
X021_D2S448_01_CpG_26.27
X021_D2S448_01_CpG_28
X021_D2S448_01_CpG_30
X021_D2S448_01_CpG_31
X028_FARP1_01_CpG_1.2
X028_FARP1_01_CpG_4
X028_FARP1_01_CpG_5.6
X028_FARP1_01_CpG_7
X028_FARP1_01_CpG_8
X028_FARP1_01_CpG_9
X028_FARP1_01_CpG_10
X028_FARP1_01_CpG_11
X028_FARP1_01_CpG_14.15.16
X028_FARP1_01_CpG_17
X028_FARP1_01_CpG_24.25
X028_FARP1_01_CpG_26.27
X028_FARP1_01_CpG_28.29
X028_FARP1_01_CpG_30
X038_GLUL_01_CpG_1
X038_GLUL_01_CpG_3
X038_GLUL_01_CpG_4
X038_GLUL_01_CpG_5.6
X038_GLUL_01_CpG_7.8.9.10
X038_GLUL_01_CpG_11
X038_GLUL_01_CpG_12
X038_GLUL_01_CpG_13.14.15.16
X038_GLUL_01_CpG_17
X054_KRT13_01_CpG_12.13.14
X054_KRT13_01_CpG_30
X066_MGC14376_01_CpG_2.3
X066_MGC14376_01_CpG_9
X066_MGC14376_01_CpG_10
X066_MGC14376_01_CpG_11.12.13
X066_MGC14376_01_CpG_16
X066_MGC14376_01_CpG_17.18
X066_MGC14376_01_CpG_19
X066_MGC14376_01_CpG_20.21
X066_MGC14376_01_CpG_22.23
X079_PIK3R4_01_CpG_4.5
X079_PIK3R4_01_CpG_6
X079_PIK3R4_01_CpG_7.8
X079_PIK3R4_01_CpG_9
X079_PIK3R4_01_CpG_10.11
X079_PIK3R4_01_CpG_12
X079_PIK3R4_01_CpG_13
X079_PIK3R4_01_CpG_14.15
X079_PIK3R4_01_CpG_16
X079_PIK3R4_01_CpG_17
X079_PIK3R4_01_CpG_18.19
X079_PIK3R4_01_CpG_22
X079_PIK3R4_01_CpG_23
X079_PIK3R4_01_CpG_24
X086_RIS1_01_CpG_3
X086_RIS1_01_CpG_4
X086_RIS1_01_CpG_5
X086_RIS1_01_CpG_6.7.8
X086_RIS1_01_CpG_9
X086_RIS1_01_CpG_12.13
X086_RIS1_01_CpG_14.15
X086_RIS1_01_CpG_16.17.18.19.20
X086_RIS1_01_CpG_23
X086_RIS1_01_CpG_24
X086_RIS1_01_CpG_25
X086_RIS1_01_CpG_26.27.28.29.30.31
X086_RIS1_01_CpG_46
X086_RIS1_01_CpG_47.48
X086_RIS1_01_CpG_49
X086_RIS1_01_CpG_50
X086_RIS1_01_CpG_51
X110_TUCAN_12_CpG_1
X110_TUCAN_12_CpG_9
X110_TUCAN_12_CpG_10
X110_TUCAN_12_CpG_11.12
X110_TUCAN_12_CpG_13
X110_TUCAN_12_CpG_15
X110_TUCAN_12_CpG_16
X110_TUCAN_12_CpG_17.18
X110_TUCAN_12_CpG_20
X110_TUCAN_12_CpG_21.22
X110_TUCAN_12_CpG_23.24
X110_TUCAN_12_CpG_25
X110_TUCAN_12_CpG_26
X124_BAALC_26_CpG_22
X139_FLI1_41_CpG_4
X139_FLI1_41_CpG_5
X139_FLI1_41_CpG_6
X139_FLI1_41_CpG_7
X139_FLI1_41_CpG_10
X139_FLI1_41_CpG_11
X139_FLI1_41_CpG_12
X139_FLI1_41_CpG_13.14
X139_FLI1_41_CpG_15.16
X139_FLI1_41_CpG_17
X139_FLI1_41_CpG_18
X139_FLI1_41_CpG_19
X139_FLI1_41_CpG_21.22
X160_SOCS1_62_CpG_1
X160_SOCS1_62_CpG_2.3.4
X160_SOCS1_62_CpG_5
X160_SOCS1_62_CpG_6.7.8
X160_SOCS1_62_CpG_15.16
X160_SOCS1_62_CpG_17.18.19.20
X160_SOCS1_62_CpG_21
X160_SOCS1_62_CpG_22
X160_SOCS1_62_CpG_23.24
X160_SOCS1_62_CpG_25
X160_SOCS1_62_CpG_26
X160_SOCS1_62_CpG_27.28
HOXA7_SQ03_CpG_1
HOXA7_SQ03_CpG_11.12
HOXA7_SQ03_CpG_13
HOXA7_SQ03_CpG_14
HOXA7_SQ03_CpG_15
HOXA7_SQ03_CpG_16
HOXA7_SQ03_CpG_17
HOXA7_SQ03_CpG_18.19
HOXA7_SQ03_CpG_21.22.23
HOXA11_SQ01_CpG_1
HOXA11_SQ01_CpG_2
HOXA11_SQ01_CpG_5.6
HOXA11_SQ01_CpG_12
HOXA11_SQ01_CpG_13.14
HOXA11_SQ01_CpG_15
HOXA11_SQ01_CpG_16
HOXA11_SQ01_CpG_17
HOXA11_SQ01_CpG_18
HOXA11_SQ01_CpG_22
HOXA11_SQ01_CpG_23
HOXA11_SQ01_CpG_24.25
HOXA11_SQ01_CpG_27
HOXA11_SQ01_CpG_28
X046_HOXB5_01_CpG_1.2
X046_HOXB5_01_CpG_3.4
X046_HOXB5_01_CpG_7.8.9
X046_HOXB5_01_CpG_10
X046_HOXB5_01_CpG_14.15
X046_HOXB5_01_CpG_16
X025_ACTG1.01_CpG_1
X025_ACTG1.01_CpG_2
X025_ACTG1.01_CpG_3.4
X025_ACTG1.01_CpG_5
X025_ACTG1.01_CpG_8.9
X025_ACTG1.01_CpG_11.12.13
X025_ACTG1.01_CpG_14
X025_ACTG1.01_CpG_15
X025_ACTG1.01_CpG_16
X025_ACTG1.01_CpG_18
X025_ACTG1.01_CpG_19
X025_ACTG1.01_CpG_20
X025_ACTG1.01_CpG_21
X025_ACTG1.01_CpG_22
X025_ACTG1.01_CpG_23
X025_ACTG1.01_CpG_24
X025_ACTG1.01_CpG_25
X025_ACTG1.01_CpG_27
X025_ACTG1.01_CpG_28
X025_ACTG1.01_CpG_29
X025_ACTG1.01_CpG_30
X003_CDKN2A_02_01_CpG_1.2
X003_CDKN2A_02_01_CpG_4
X003_CDKN2A_02_01_CpG_5
X003_CDKN2A_02_01_CpG_8.9.10.11.12.13
X003_CDKN2A_02_01_CpG_15.16.17
X003_CDKN2A_02_01_CpG_18
X003_CDKN2A_02_01_CpG_19
X003_CDKN2A_02_01_CpG_20.21
X003_CDKN2A_02_01_CpG_22.23.24.25
X003_CDKN2A_02_01_CpG_26
X004_RPL22_001_CpG_1.2
HOXA3_SQ01_CpG_1
HOXA3_SQ01_CpG_2
HOXA3_SQ01_CpG_3
HOXA3_SQ01_CpG_7.8
HOXA3_SQ01_CpG_9.10.11.12
HOXA3_SQ01_CpG_13.14
HOXA3_SQ01_CpG_15.16
HOXA3_SQ01_CpG_17
HOXA3_SQ01_CpG_19
HOXA3_SQ01_CpG_20
HOXA3_SQ01_CpG_21.22
HOXA3_SQ01_CpG_23
HOXA3_SQ01_CpG_24
HOXA3_SQ01_CpG_25.26
HOXA3_SQ01_CpG_28
HOXA3_SQ01_CpG_29
HOXA3_SQ01_CpG_30
HOXA3_SQ01_CpG_31.32.33
HOXA3_SQ01_CpG_35.36
HOXA3_SQ01_CpG_37.38.39
HOXA3_SQ01_CpG_40
HOXA3_SQ01_CpG_42.43
HOXA3_SQ01_CpG_44.45
SATalpha_CpG_5
SATalpha_CpG_8
SATalpha_CpG_10
SATalpha_CpG_19
SATalpha_CpG_21.22
HOXA3_ABO1_CpG_4
HOXA3_ABO1_CpG_5
HOXA3_ABO1_CpG_6.7
HOXA3_ABO1_CpG_12.13.14
HOXA3_ABO1_CpG_16.17
HOXA3_ABO1_CpG_18.19.20
HOXA3_ABO1_CpG_21
HOXA3_ABO1_CpG_23.24
HOXA3_ABO1_CpG_25.26
X022_DAPK1_01_CpG_1.2
X022_DAPK1_01_CpG_3
X022_DAPK1_01_CpG_4.5
X022_DAPK1_01_CpG_7.8
X022_DAPK1_01_CpG_9.10
X022_DAPK1_01_CpG_11.12
X022_DAPK1_01_CpG_13.14
X022_DAPK1_01_CpG_15.16.17
X022_DAPK1_01_CpG_18
X022_DAPK1_01_CpG_19
X022_DAPK1_01_CpG_20
X022_DAPK1_01_CpG_21
X022_DAPK1_01_CpG_22
X022_DAPK1_01_CpG_23.24
X022_DAPK1_01_CpG_28.29.30.31.32
X022_DAPK1_01_CpG_36.37
X022_DAPK1_01_CpG_38
X022_DAPK1_01_CpG_39
X022_DAPK1_01_CpG_41
X022_DAPK1_01_CpG_42.43
X023_ACTG1.02_CpG_1
X023_ACTG1.02_CpG_2
X023_ACTG1.02_CpG_3
X023_ACTG1.02_CpG_4
X023_ACTG1.02_CpG_7
X023_ACTG1.02_CpG_8.9.10.11
X023_ACTG1.02_CpG_12
X023_ACTG1.02_CpG_13
X023_ACTG1.02_CpG_14
X023_ACTG1.02_CpG_15
X023_ACTG1.02_CpG_16.17
X023_ACTG1.02_CpG_18.19
X023_ACTG1.02_CpG_21.22
X031_FHL2_01_CpG_3
X031_FHL2_01_CpG_11
X031_FHL2_01_CpG_13.14
X031_FHL2_01_CpG_15.16
X031_FHL2_01_CpG_17
X031_FHL2_01_CpG_18.19
X031_FHL2_01_CpG_20
X031_FHL2_01_CpG_21
X031_FHL2_01_CpG_23
X015_CD3D_01_CpG_1
X015_CD3D_01_CpG_2
X015_CD3D_01_CpG_3
X015_CD3D_01_CpG_4
X015_CD3D_01_CpG_5
X015_CD3D_01_CpG_6
X015_CD3D_01_CpG_7
X015_CD3D_01_CpG_8
X015_CD3D_01_CpG_9
X015_CD3D_01_CpG_10.11.12.13
X015_CD3D_01_CpG_14.15.16.17
X015_CD3D_01_CpG_18.19
X015_CD3D_01_CpG_21
X015_CD3D_01_CpG_22
X015_CD3D_01_CpG_23.24
X015_CD3D_01_CpG_25.26.27

TABLE 10
FIG. 6A y-axis: Sample references (bottom to top)

103_02KM1932
005_AML_014
093_02KM896
028_AML_118
084_AML_114
011_AML_048
081_AML_098
050_AML_053
073_AML_001
017_AML_077
021_AML_070
001_AML_046
013_AML_100
037_AML_086
083_AML_016
089_AML_095
047_AML_030
030_AML_117
061_AML_002
063_AML_029
088_AML_060
039_AML_079
031_AML_004
093_AML_105
119_98KM795
002_98PB287
043_01PB382
061_01KM1523
068_01KM2189
083_02KM90
084_02KM183
048_01KM637
088_02KM706
047_01KM496
053_01PB1072
108_02KM2242
085_02KM255
077_01KM2621
114_02KM2732
064_01KM1983
081_01KM3062
007_AML_115
102_02KM1746
079_01PB2846
080_01KM3019
040_AML_045
029_00PB262
069_AML_012
029_AML_009
085_AML_011
009_AML_040
055_AML_028
075_AML_057
099_02KM1567
006_AML_104
026_AML_119
049_AML_068
056_AML_111
064_AML_083
065_AML_069
079_AML_055
092_AML_008
033_AML_013
060_AML_074
003_AML_109
045_AML_091
010_AML_035
034_AML_085
054_AML_020
023_99PB1458
069_01KM2331
016_99KM831
039_00KM2370
049_01PB694
063_01KM1951
054_01PB1111
066_01KM2148
015_99KM563
028_00PB369
051_01KM914
113_02KM2620
048_AML_064
076_AML_089
068_AML_099
072_AML_088
014_AML_103
016_AML_065
024_AML_110
038_AML_050
035_AML_097
074_AML_090
060_01KM1396
075_01KM2605
086_02KM421
107_02KM2188
013_99PB312
062_01PB1806
097_02KM1414
070_01KM2472
087_02KM429
098_02KM1514
018_99KM915
017_99KM859
104_02KM2018
038_00KM2221
050_01KM849
110_02PB2391
067_01PB2129
082_02KM4
041_01KM321
012_99PB303
115_02PB2822
117_98PB589
094_02PB1130
121_99KM334
112_02KM2564
101_02KM1644
120_98KM798
001_98PB199
020_99PB1142
076_01PB2625
058_01KM1273
096_02KM1407
074_01KM2551
056_01KM1105
111_02KM2472
040_00KM2459
106_02KM2061
027_00KM331
031_00PB785
100_02KM1573
044_01KM459
026_00PB285
042_01KM376
118_98KM785
005_98PB834
095_02KM1236
022_99PB1321
046_01KM452
032_00KM856
009_98KM1178
037_00PB1729
090_02PB786
019_AML_059
086_AML_031
032_AML_049
080_AML_071
058_AML_108
090_AML_073
022_AML_116
015_AML_081
070_AML_084
077_AML_041
023_AML_094
094_AML_044
082_AML_025
059_01PB1332
051_AML_042
057_AML_018
042_AML_015
025_AML_026
095_AML_039
071_AML_021
002_AML_027
066_AML_010
067_AML_072
053_AML_043
091_AML_007
044_AML_058
020_AML_022
059_AML_023
096_AML_047
036_AML_092
087_AML_107
008_AML_017
012_AML_038
046_AML_078
078_AML_112
034_00KM1115
052_AML_093
004_AML_005
027_AML_087

TABLE 11

| | | | | Sex | |
|---|---|---|---|---|---|
| | Cytogenetic | | | 1 = m | Age |
| Sample ID | Group | Karyotype | FISH | 2 = f | years |
| 073_AML_001 | complex karyotype | 48, XX, +8, add(9)(q34), +13 | 8 | 2 | 40 |
| 066_AML_010 | normal karyotype | 46, XY | negative | 1 | 34 |
| 013_AML_100 | inv(16) | 46, XX, inv(16)(p13q22) | inv(16) | 2 | 46 |
| 041_AML_101 | t(15; 17) | 46, XY, t(15; 17)(q22; q21) | t(15; 17) | 1 | 41 |
| 014_AML_103 | inv(16) | 46, XY, inv(16)(p13q22) | inv(16) | 1 | 50 |
| 006_AML_104 | t(15; 17) | 46, XY, t(15; 17)(q22; q21) | t(15; 17), +8 | 1 | 43 |
| 093_AML_105 | t(8; 21) | 45, X, −Y, t(8: 21)(q22; q22) | no inv(16), no | 1 | 48 |
| | | | t(11q23) | | |
| 087_AML_107 | 8 | 47, XY, +8 | 8 | 1 | 59 |
| 058_AML_108 | normal karyotype | 46, XX | negative | 2 | 68 |
| 003_AML_109 | normal karyotype | 46, XY | negative | 1 | 48 |
| 085_AML_011 | normal karyotype | 46, XX | negative | 2 | 47 |
| 024_AML_110 | normal karyotype | 46, XY | no inv(16), no | 1 | 56 |
| | | | t(11q23) | | |
| 056_AML_111 | t(9; 11) | 46, XX, t(9; 11)(p22; q23) | t(9; 11) | 2 | 48 |
| 078_AML_112 | normal karyotype | 46, XY | negative | 1 | 66 |
| 084_AML_114 | t(8; 21) | 46, XY, t(8; 21)(q22; q22) | t(8; 21) | 1 | 50 |
| 007_AML_115 | normal karyotype | 46, XY | negative | 1 | 28 |
| 022_AML_116 | normal karyotype | 46, XX | negative | 2 | 52 |
| 030_AML_117 | t(15; 17) | 46, XY, t(15; 17)(q22; q21) | no inv(16), no | 1 | 40 |
| | | | t(11q23) | | |
| 028_AML_118 | normal karyotype | 46, XY | NA | 1 | 51 |
| 026_AML_119 | t(15; 17) | 46, XX, t(15; 17)(q22; q21) | t(15; 17) | 2 | NA |
| 069_AML_012 | normal karyotype | 46, XY | negative | 1 | 31 |
| 033_AML_013 | normal karyotype | 46, XX | negative | 2 | 73 |
| 005_AML_014 | complex karyotype | 47, XY, del(5q), del(7q), abn | del(5)(q31-q33), | 1 | 35 |
| | | (11q23), add(12)(p13), +22 | ?t(11q23), +12p | | |
| 042_AML_015 | normal karyotype | 46, XY | negative | 1 | 63 |
| 083_AML_016 | t(8; 21) | 46, XX, t(8; 21)(q22; q22) | t(8; 21) | 2 | 49 |
| 008_AML_017 | normal karyotype | 46, XY | negative | 1 | 46 |
| 057_AML_018 | other | 46, XY, t(11; 15)(p15; q11), t | negative | 1 | 28 |
| | | (12; 20)(p11.2; q11.2) | | | |
| 061_AML_002 | t(15; 17) | 46, XY, t(15; 17)(q22; q21) | NA | 1 | 35 |
| 054_AML_020 | t(9; 11) | 46, X, −Y, | t(9; 11) | 1 | 65 |
| | | +8, t(9; 11)(p22; q23) | | | |
| 071_AML_021 | t(9; 11) | 46, XY, t(9; 11)(p22; q23) | t(9; 11) | 1 | 23 |
| 020_AML_022 | normal karyotype | 46, XY | NA | 1 | 52 |
| 059_AML_023 | t(8; 21) | 46, XY, t(8; 21)(q22; q22) | t(8; 21) | 1 | 73 |
| 082_AML_025 | inv(16) | 46, XY, inv(16)(p13q22) | inv(16) | 1 | 67 |
| 025_AML_026 | t(8; 21) | 45, X, −Y, t(8: 21)(q22; q22) | NA | 1 | 72 |
| 002_AML_027 | normal karyotype | 46, XY | negative | 1 | 47 |
| 055_AML_028 | normal karyotype | 46, XX | negative | 2 | 69 |
| 063_AML_029 | t(15; 17) | 46, XY, t(15; 17)(q22; q21) | t(15; 17) | 1 | 50 |
| 047_AML_030 | normal karyotype | 47, XY | negative | 1 | 31 |
| 086_AML_031 | 8 | 48, XY, +8 | 8 | 1 | 60 |
| 018_AML_034 | inv(16) | 46, XX, inv(16)(p13q22) | inv(16) | 2 | 42 |
| 010_AML_035 | normal karyotype | 46, XX | negative | 2 | 40 |
| 012_AML_038 | t(8; 21) | 46, XY, t(8; 21)(q22; q22) | t(8; 21) | 1 | 58 |
| 095_AML_039 | normal karyotype | 46, XX | negative | 2 | 63 |
| 031_AML_004 | t(8; 21) | 46, XY, t(8; 21)(q22; q22) | no inv(16), no | 1 | 41 |
| | | | t(11q23) | | |
| 009_AML_040 | normal karyotype | 46, XY | negative | 1 | 59 |
| 077_AML_041 | other | 46, XY, t(4; 15)(q13; q21), inv | NA | 1 | 67 |
| | | (9)(p12q12) | | | |
| 051_AML_042 | normal karyotype | 46, XY | negative | 1 | 69 |
| 053_AML_043 | normal karyotype | 46, XX | negative | 2 | 62 |
| 094_AML_044 | t(8; 21) | 46, XY, t(6; 8)(q25; q22), add | t(8; 21) | 1 | 59 |
| | | (19)(q13) | | | |
| 040_AML_045 | normal karyotype | 46, XX | negative | 2 | 47 |
| 001_AML_046 | normal karyotype | 46, XX | negative | 2 | 31 |
| 096_AML_047 | inv(3)/t(3; 3) | 45, XX, inv(3), −7 | no inv(16), no | 2 | 35 |
| | | | t(11q23) | | |
| 011_AML_048 | t(8; 21) | 46, XY, t(8; 21)(q22; q22) | t(8; 21) | 1 | NA |
| 032_AML_049 | normal karyotype | 46, XX | no inv(16), no | 2 | NA |
| | | | t(11q23) | | |
| 004_AML_005 | complex karyotype | 46, XY, t(2; 8), add(7)(q32), ? | +8, ?t(11q23) | 1 | 39 |
| | | t(10; 11; 19) | | | |
| 038_AML_050 | t(9; 11) | 46, XY, t(9; 11)(p22; q23) | t(9; 11) | 1 | 48 |
| 050_AML_053 | inv(16) | 47, XX, inv(16)(p13q22), +8 | inv(16), +8 | 2 | 45 |
| 079_AML_055 | normal karyotype | 46, XY | negative | 1 | 33 |
| 062_AML_056 | normal karyotype | 46, XX | negative | 2 | 50 |
| 075_AML_057 | normal karyotype | 46, XY | negative | 1 | 72 |
| 044_AML_058 | complex karyotype | 45, XX, der(5)t(5; ?; 15)(q31; | NA | 2 | 63 |
| | | ?; q11), del(7)(q11), −15 | | | |
| 019_AML_059 | other | 47, XX, del(1)(p13p36), der | NA | 2 | 31 |
| | | (10; 11)(p15; q13) | | | |
| 088_AML_060 | t(15; 17) | 46, XY, t(15; 17)(q22; q21) | t(15; 17), +8 | 1 | 66 |
| 043_AML_062 | t(8; 21) | 46, XY, t(8; 21)(q22; q22) | NA | 1 | 72 |
| 048_AML_064 | normal karyotype | 46, XX | NA | 2 | 35 |
| 016_AML_065 | inv(16) | 46, XY, inv(16)(p13q22) | NA | 1 | 32 |
| 049_AML_068 | t(9; 11) | 47, XX, +8, t(9; 11)(p22; q23) | t(9; 11), +8 | 2 | 53 |
| 065_AML_069 | t(9; 11) | 47, XX, +8, t(9; 11)(p22; q23) | t(9; 11) | 2 | 37 |
| 091_AML_007 | normal karyotype | 46, XX | no inv(16), no | 2 | 38 |
| | | | t(11q23) | | |
| 021_AML_070 | inv(16) | 46, XX, inv(16)(p13q22) | inv(16) | 2 | 48 |
| 080_AML_071 | other | 47, XX, +11 | 11 | 2 | 61 |
| 067_AML_072 | complex karyotype | 47, X, −Y, | +8, +12p, +17p, +22 | 1 | 65 |
| | | +6, +8, add(12)(p13), del | | | |
| | | (16)(p11), add(17)(q23), add | | | |
| | | (22)(q13) | | | |
| 090_AML_073 | inv(16) | 46, XX, inv(16)(p13q22) | inv(16) | 2 | 64 |
| 060_AML_074 | normal karyotype | 46, XX | negative | 2 | 68 |
| 017_AML_077 | inv(16) | 46, XX, inv(16)(p13q22) | inv(16) | 2 | 22 |
| 046_AML_078 | complex karyotype | 52, XY, +1, −5, | NA | 1 | 73 |
| | | +11, +11, +11, +18, +18, +22 | | | |
| 039_AML_079 | inv(16) | 46, XY, inv(16)(p13q22), +8 | inv(16), +8 | 1 | 52 |
| 092_AML_008 | normal karyotype | 46, XY | negative | 1 | 69 |
| 015_AML_081 | inv(16) | 46, XX, inv(16)(p13q22) | inv(16) | 2 | 40 |
| 064_AML_083 | t(9; 11) | 46, XX, t(9; 11)(p22; q23) | t(9; 11) | 2 | 34 |
| 070_AML_084 | inv(16) | 48, XX, +8, +13, inv(16)(p13q22) | inv(16), +8, +21 | 2 | 34 |
| 034_AML_085 | normal karyotype | 46, XY | negative | 1 | 69 |
| 037_AML_086 | t(15; 17) | 46, XY, t(15; 17)(q22; q21) | t(15; 17) | 1 | 43 |
| 027_AML_087 | normal karyotype | 46, XY | negative | 1 | 57 |
| 072_AML_088 | t(9; 11) | 46, XX, t(9; 11)(p22; q23) | NA | 2 | 60 |
| 076_AML_089 | inv(16) | 46, XX, inv(16)(p13q22) | inv(16) | 2 | 36 |
| 029_AML_009 | normal karyotype | 46, XY | no inv(16), no | 1 | 34 |
| | | | t(11q23) | | |
| 074_AML_090 | inv(16) | 46, XY, inv(16)(p13q22) | inv(16) | 1 | 33 |
| 045_AML_091 | normal karyotype | 46, XY | NA | 1 | 67 |
| 036_AML_092 | complex karyotype | 46, XY, add(2)(p13), −5, | del(7q22-q35), | 1 | 65 |
| | | del(7)(q22), add(9)(p13), | del(17p), del | | |
| | | −13 | (20q) | | |
| 052_AML_093 | normal karyotype | 46, XY | negative | 1 | 63 |
| 023_AML_094 | normal karyotype | 46, XX | negative | 2 | 34 |
| 089_AML_095 | t(15; 17) | 46, XX, t(15; 17)(q22; q21) | t(15; 17) | 2 | 64 |
| 035_AML_097 | inv(16) | 46, XY, inv(16)(p13q22) | inv(16) | 1 | 37 |
| 081_AML_098 | t(8; 21) | 45, X, −X, t(8; 21) | t(8; 21) | 2 | 45 |
| 068_AML_099 | t(15; 17) | 46, XX, t(15; 17)(q22; q21) | t(15; 17) | 2 | NA |
| 1_98PB199 | normal karyotype | 46, XX | negative | 2 | 39 |
| 2_98PB287 | t(15; 17) | 47, XXY, t(15; 17)(q22; q21) | t(15; 17), +X | 1 | 38 |
| 5_98PB834 | normal karyotype | 46, XY | negative | 1 | 43 |
| 9_98KM1178 | normal karyotype | 46, XX | negative | 2 | 38 |
| 12_99PB303 | normal karyotype | 46, XY | negative | 1 | 60 |
| 13_99PB312 | normal karyotype | 46, XY | negative | 1 | 55 |
| 15_99KM563 | other | 46, XX, t(8; 16)(p11; p13) | inv(16) (25.2%) | 2 | 33 |
| 16_99KM831 | normal karyotype | 46, XX | negative | 2 | 46 |
| 17_99KM859 | t(15; 17) | 46, XY, t(15; 17)(q22; q21) | t(15; 17) (44.1%) | 1 | 61 |
| 18_99KM915 | complex karyotype | complex | negative | 1 | 60 |
| 20_99PB1142 | normal karyotype | 46, XX | negative | 2 | 52 |
| 21_99KM1241 | normal karyotype | 46, XX | negative | 2 | 35 |
| 22_99PB1321 | normal karyotype | 46, XX | negative | 2 | 42 |
| 23_99PB1458 | normal karyotype | 46, XY | negative | 1 | 45 |
| 26_00PB285 | Other | 51, XY, +13, +13, +, 13, +13, | ND | 1 | 45 |
| | | +13 (Pentasomie 13) | | | |
| 27_00KM331 | normal karyotype | 46, XX | negative | 2 | 35 |
| 28_00PB369 | inv(16) | 45, X, −Y, inv(16)(p13; q22) | inv(16) (35.5%) | 1 | 55 |
| 29_00PB262 | normal karyotype | 46, XY[21] in Kiel | negative | 1 | 38 |
| 31_00PB785 | normal karyotype | 46, XY[12] | negative | 1 | 58 |
| 32_00KM856 | normal karyotype | 46, XX[16] | negative | 2 | 57 |
| 34_00KM1115 | inv(16) | 46, XY, inv(16)(p13q22)[17] | negative | 1 | 32 |
| | | in Kiel | | | |
| 37_00PB1729 | normal karyotype | 46, XY[1] | negative | 1 | 38 |
| 38_00KM2221 | normal karyotype | 46, XY[10] | negative | 1 | 24 |
| 39_00KM2370 | normal karyotype | 46, XY[18] | negative, ? +8 | 1 | 35 |
| | | | (9.7% 4 Sig.) | | |
| 40_00KM2459 | normal karyotype | 46, XX[7] | negative | 2 | 51 |
| 41_01KM321 | normal karyotype | 46, XY[12] | negative | 1 | 54 |
| 42_01KM376 | inv(3)/t(3; 3) | 45, XY, inv(3)(q21q26), −7, | negative | 1 | 52 |
| | | inv(9)p11q13)c[10] | | | |
| 43_01PB382 | t(15; 17) | 46, XY, t(X; 17)(q13; q21), t | t(15; 17) (46.7%) | 1 | 37 |
| | | (15; 17)(q22; q21)[16] | | | |
| | | 46, XY[1] | | | |
| 44_01KM459 | other | 48, XX, +8, +19[12] | ND | 2 | 57 |
| 46_01KM452 | other | 47, XY, +8, t(9; 22)(q34; q11) | negative | 1 | 39 |
| | | [21] in Dusseldorf | | | |
| 47_01KM496 | complex karyotype | 46, XY[14] | negative | 1 | 28 |
| | | 46, XY, +9, −18, +mar[3] | | | |
| 48_01KM637 | t(8; 21) | 45, X, −Y, | negative | 1 | 43 |
| | | t(8; 21)(q22; q22)[11] | | | |
| 49_01PB694 | normal karyotype | 46, XX[10] | negative | 2 | 43 |
| 50_01KM849 | other | 46, XX, t(11; 20)(p15; q11)[6] | negative | 2 | 34 |
| 51_01KM914 | inv(16) | 46, XY, inv(16)(q13q22)[6) | inv(16) (23.5%) | 1 | 35 |
| | | in Dusseldorf | | | |
| 53_01PB1072 | normal karyotype | 46, XX[17] | negative | 2 | 52 |
| 54_01PB1111 | inv(16) | 46, XX, | inv(16) (25.3%) | 2 | 38 |
| | | inv(16)(p13q22)[12] | | | |
| | | 46, XX, del(7)(q22), | | | |
| | | inv(16)(p13q22)[2] | | | |
| 56_01KM1105 | normal karyotype | 46, XX[9] | negative | 2 | 46 |
| 57_01PB1172 | normal karyotype | 46, XY[5] | negative | 1 | 57 |
| 58_01KM1273 | other | 46, XY, inv(9)(p11q13)c[7] | negative | 1 | 31 |
| | | 47, XY, inv(9)(p11q13)c, +19 | | | |
| | | [6] | | | |
| 59_01PB1332 | t(8; 21) | 46, XY, t(8; 21)(q22; q22)[2] | negative | 1 | 49 |
| | | 46, XY, del(9)(q13; q22), t(8; | | | |
| | | 21)(q22; q22)[5] | | | |
| 60_01KM1396 | normal karyotype | 46, XY[16] | negative | 1 | 49 |
| 61_01KM1523 | t(15; 17) | 46, XX[2] | negative | 2 | 40 |
| | | 47, XX + 8, t(15; 17)(q22; q22), | | | |
| | | i(17)(q10)[7] | | | |
| 62_01PB1806 | normal karyotype | 46, XX[12] | negative | 2 | 36 |
| 63_01KM1951 | inv(16) | 46, XX, inv(16)(p13q22)[15] | ND | 2 | 22 |
| 64_01KM1983 | complex karyotype | 46, XY, del(5)(q23q31)[1]44, | negative | 1 | 59 |
| | | XY, del(5)(q23q31), −7, −17, | | | |
| | | add(19)(p13), −20, | | | |
| | | +mar[2]43, XY, del(5)(q23q31), | | | |
| | | −7, −14, −17, | | | |
| | | add(19)(p13), −20, | | | |
| | | +mar[11] | | | |
| 66_01KM2148 | normal karyotype | 46, XX[7] | negative | 2 | 16 |
| 67_01PB2129 | t(15; 17) | 46, XX, t(15; 17)(q21; q21)[15] | ND | 2 | 49 |
| 68_01KM2189 | t(8; 21) | 46, XX[5] | negative | 2 | 24 |
| | | 46, XX, del(7)q22), t(8; 21)(q22; | | | |
| | | q22)[2] | | | |
| | | 45, XX, del(7)(q22), t(8; 21) | | | |
| | | (q22; q22), −9[7] | | | |
| 69_01KM2331 | complex karyotype | ~59, X, −X, −X, −1, −3, −4, −5, −6, −7, | t(11q23) (70.7%) | 2 | 59 |
| | | −12, −14, −16, −17, | | | |
| | | 2xadd(21)(p11), +2mar | | | |
| 70_01KM2472 | t(15; 17) | 46, XY, t(15; 17)(q22; q21)[15] | negative | 1 | 54 |
| 71_01KM2509 | del(9q) | 46, XY[1] | negative | 1 | 39 |
| | | 46, XY, del(9)(q22q34)[5] | | | |
| | | 47, XY, del(9)(q22q34), +21 | | | |
| | | [5] | | | |
| 74_01KM2551 | t(15; 17) | 46, XX, del(7)(q31q36), t(15; | negative | 2 | 29 |
| | | 17)(q22; q21)[19] | | | |
| 75_01KM2605 | t(11q23) | 45, X, −Y, | t(11q23) (63.7%) | 1 | 39 |
| | | t(11; 19)(q23; p13)[23] | | | |
| 76_01PB2625 | normal karyotype | 46, XX[5] | negative | 2 | 60 |
| 77_01KM2621 | normal karyotype | 46, XX[10] | negative | 2 | 33 |
| 79_01PB2846 | t(9; 11) | 46, XX, t(9; 11)(p22; q23)[3] | t(11q23) (42.5%) | 2 | 32 |
| | | 47, XX, t(9; 11)(p22; q23), +13 | | | |
| | | [8] | | | |
| 80_01KM3019 | normal karyotype | 46, XX[10] | negative | 2 | 47 |
| 81_01KM3062 | normal karyotype | 46, XX[8] | negative | 2 | 24 |
| 82_02KM4 | normal karyotype | 46, XX[20] | negative | 2 | 35 |
| 83_02KM90 | t(15; 17) | 46, XX, t(15; 17)(q22; q21)[8] | negative | 2 | 54 |
| | | 46, XX, del(7)(q22), t(15; 17) | | | |
| | | (q22; q21)[4] | | | |
| 84_02KM183 | t(8; 21) | 46, XX, t(8; 21)(q22; q22)[3] | ND | 2 | 40 |
| | | 45, X, −X, | | | |
| | | t(8; 21)(q22; q22)[7] | | | |
| 85_02KM255 | normal karyotype | 46, XX[13] | negative | 2 | 58 |
| 86_02KM421 | normal karyotype | 46, XY | negative | 1 | 41 |
| 87_02KM429 | t(15; 17) | 46, XY[6] | negative | 1 | 45 |
| | | 47, XY, +8, t(15; 17)(q22; q21) | | | |
| | | [10] | | | |
| 88_02KM706 | t(8; 21) | 46, XY, t(8; 21)(q22; q22)[13] | negative | 1 | 19 |
| 90_02PB786 | normal karyotype | 46, XX[10] | negative | 2 | 55 |
| 92_02KM883 | normal karyotype | 46, XY[12] | negative | 1 | 50 |
| 93_02KM896 | normal karyotype | 46, XY[12] | negative | 1 | 38 |
| 94_02PB1130 | complex karyotype | complex karyotype | t(11q23) (77%) | 2 | 57 |
| 95_02KM1236 | other | 46, XY, t(5; 12)(q31; q13)[17] | negative | 1 | 19 |
| 96_02KM1407 | inv(3)/t(3; 3) | 45, XX, t(3; 3)(q21; q26), −7 | negative | 2 | 36 |
| | | [10] | | | |
| 97_02KM1414 | t(11q23) | 46, XX, t(6; 11)(q25; q23)[14] | t(11q23) (88.5%) | 2 | 58 |
| 98_02KM1514 | t(15; 17) | 46, XY, t(15; 17)(q22; q21)[13] | negative | 1 | 43 |
| | | 46, XY, t(15; 17)(q22; q21), iso | | | |
| | | (17)(q10)[2] | | | |
| 99_02KM1567 | normal karyotype | 46, XX[14] | negative | 2 | 47 |
| 100_02KM1573 | normal karyotype | 46, XY[10] | negative | 1 | 53 |
| 101_02KM1644 | complex karyotype | 45, XX, del(4)(q25), del(5)(q13q33), | t(11q23) (85%) | 2 | 50 |
| | | add(9)(p24), +11, −17, | | | |
| | | −18[20] | | | |
| 102_02KM1746 | normal karyotype | 46, XY[15] | negative | 1 | 41 |
| 103_02KM1932 | normal karyotype | 46, XX[18] in Kiel | negative | 2 | 29 |
| 104_02KM2018 | t(11q23) | 46, XY, t(4; 11; 9)(q27; q23; p22) | t(11q23) | 1 | 51 |
| | | [20] | (52%)/AF9pool | | |
| | | | (49%) | | |
| 105_02KM2037 | inv(16) | 46, XY, del(7)(q32), inv(16 | inv(16) (29%), | 1 | 23 |
| | | (p13q22)[5] | del(7q35) | | |
| | | 47, XY, del(7)(q32), inv(16 | (96.9%) | | |
| | | (p13q22), +22[10] | | | |
| | | 46, XY[1] | | | |
| 106_02KM2061 | t(6; 9) | 46, XX, t(6; 9)(p23; q34)[10] | negative | 2 | 52 |
| 107_02KM2188 | normal karyotype | 46, XY[16] | negative | 1 | 50 |
| 108_02KM2242 | normal karyotype | 46, XX[6] | negative | 2 | 41 |
| 110_02PB2391 | 8 | 46, XY[2] | negative | 1 | 56 |
| | | 47, XY, +8[11] | | | |
| 111_02KM2472 | 8 | 46, XY[17] | negative; ? +8 | 1 | 60 |
| | | 47, XY, +8[1] | (37%) | | |
| 112_02KM2564 | normal karyotype | 46, XX[14] | negative; ? | 2 | 24 |
| | | | inv(16) | | |
| 113_02KM2620 | inv(16) | 46, XY, inv(16)(p13q22)[13] | inv(16) (20%) | 1 | 52 |
| | | 47, XY, +8, inv(16)(p13q22) | | | |
| | | [3] | | | |
| 114_02KM2732 | normal karyotype | 46, XX[11] | negative | 2 | 23 |
| 115_02PB2822 | inv(3)/t(3; 3) | 45, XX, inv(3)(q21q26), −7 | negative | 2 | 56 |
| | | [17] | | | |
| 116_02PB3234 | t(15; 17) | 46, XX, t(2; 7)(p21; q22), t(4; | ND | 2 | 52 |
| | | 10)(q31; q22), t(15; 17)(q22; | | | |
| | | q21)[15] | | | |
| 117_98PB589 | normal karyotype | 46, XY | ND | 1 | 68 |
| 118_98KM785 | normal karyotype | 46, XX | ND | 2 | 69 |
| 119_98KM795 | t(15; 17) | 46, XX, t(15; 17)(q22; q21) | ND | 2 | 62 |
| 120_98KM798 | normal karyotype | 46, XX | ND | 2 | 62 |
| 121_99KM334 | normal karyotype | 46, XX | ND | 2 | 61 |
| 122_99KM703 | normal karyotype | 46, XY | ND | 1 | 63 |
| 123_99KM723 | other | 46, XX, del(16)(q12) | ND | 2 | 71 |
| 124_99KM1005 | normal karyotype | 46, XY | del(5q31) (84%), | 1 | 74 |
| | | | del(7q22) (98%), | | |
| | | | del(7q35) (95%), | | |
| | | | del(12p) (96%) | | |
| 125_00PB361 | normal karyotype | 46, XX | negative | 2 | 63 |

| | | | FAB | | | | |
|---|---|---|---|---|---|---|---|
| | Preceding | Sample | (0 = M0 | CR/PR | Current | | Surv |
| Sample ID | Malignancy | source | etc.) | after ICE | Remission | Status | Days |
| 073_AML_001 | no | PB | 2 | CR | Relapse | dead | 215 |
| 066_AML_010 | NA | BM | 5 | RD | Relapse | dead | 235 |
| 013_AML_100 | no | PB | 4 | CR | CR | alive | 2103 |
| 041_AML_101 | no | BM | 3 | CR | CR | alive | 2020 |
| 014_AML_103 | no | PB | 4 | CR | Relapse | dead | 435 |
| 006_AML_104 | no | PB | 3 | ED | ED | dead | 3 |
| 093_AML_105 | no | PB | 2 | CR | CR | alive | 985 |
| 087_AML_107 | no | BM | 5 | ED | ED | dead | 8 |
| 058_AML_108 | no | BM | NA | RD | RD | dead | 684 |
| 003_AML_109 | no | BM | 4 | CR | CR | alive | 1957 |
| 085_AML_011 | no | BM | 1 | RD | Relapse | dead | 206 |
| 024_AML_110 | no | BM | 4 | CR | Relapse | dead | 322 |
| 056_AML_111 | yes | BM | 5 | CR | TD | dead | 284 |
| 078_AML_112 | no | BM | 4 | CR | RD | dead | 536 |
| 084_AML_114 | no | PB | 2 | CR | CR | alive | 1066 |
| 007_AML_115 | no | BM | 2 | CR | 2CR | alive | 2158 |
| 022_AML_116 | no | BM | 4 | CR | CR | dead | 699 |
| 030_AML_117 | no | PB | 3 | ED | ED | dead | 1 |
| 028_AML_118 | no | PB | 2 | CR | HD | dead | 101 |
| 026_AML_119 | no | BM | 3 | ED | ED | dead | 1 |
| 069_AML_012 | no | PB | 5 | CR | CR | alive | 1284 |
| 033_AML_013 | yes | PB | 4 | CR | Relapse | dead | 85 |
| 005_AML_014 | no | BM | 1 | RD | Relapse | dead | 722 |
| 042_AML_015 | no | PB | 2 | RD | CR | dead | 489 |
| 083_AML_016 | no | BM | 2 | CR | 2Relapse | alive | 1152 |
| 008_AML_017 | no | PB | 1 | PR | Relapse | dead | 580 |
| 057_AML_018 | no | PB | 1 | CR | Relapse | dead | 330 |
| 061_AML_002 | no | BM | 3 | CR | CR | alive | 1170 |
| 054_AML_020 | no | PB | 5 | CR | CR | alive | 1164 |
| 071_AML_021 | yes | BM | 5 | ED | ED | dead | 22 |
| 020_AML_022 | no | PB | 0 | RD | RD | dead | 155 |
| 059_AML_023 | no | BM | NA | ED | NA | dead | 29 |
| 082_AML_025 | no | BM | 2 | CR | Relapse | alive | 1169 |
| 025_AML_026 | no | PB | 4 | RD | | dead | 162 |
| 002_AML_027 | no | BM | 5 | RD | Relapse | dead | 330 |
| 055_AML_028 | NA | BM | NA | PR | NA | dead | 80 |
| 063_AML_029 | no | PB | 3 | CR | Relapse | dead | 740 |
| 047_AML_030 | no | BM | 2 | CR | CR | alive | 1740 |
| 086_AML_031 | yes | BM | 2 | PR | RD | dead | 343 |
| 018_AML_034 | NA | BM | 4 | CR | CR | alive | 1026 |
| 010_AML_035 | no | PB | 4 | CR | CR | alive | 1161 |
| 012_AML_038 | no | PB | 2 | RD | Relapse | dead | 339 |
| 095_AML_039 | no | BM | 1 | PR | CR | dead | 149 |
| 031_AML_004 | no | BM | 2 | CR | Relapse | dead | 214 |
| 009_AML_040 | no | PB | 4 | PR | Relapse | dead | 218 |
| 077_AML_041 | NA | PB | 1 | CR | Relapse | dead | 547 |
| 051_AML_042 | no | BM | 4 | RD | RD | dead | 31 |
| 053_AML_043 | no | PB | 4 | CR | CR | alive | 1543 |
| 094_AML_044 | no | PB | 2 | CR | CR | alive | 927 |
| 040_AML_045 | no | PB | 2 | PR | Relapse | dead | 295 |
| 001_AML_046 | no | PB | 2 | CR | CR | dead | 673 |
| 096_AML_047 | NA | PB | 1 | RD | RD | dead | 478 |
| 011_AML_048 | NA | BM | NA | CR | CR | alive | 1332 |
| 032_AML_049 | NA | PB | NA | RD | CR | dead | 669 |
| 004_AML_005 | no | BM | 1 | RD | RD | dead | 248 |
| 038_AML_050 | no | BM | 5 | CR | CR | alive | 1910 |
| 050_AML_053 | NA | PB | 4 | PR | CR | alive | 1502 |
| 079_AML_055 | no | PB | 4 | RD | Relapse | dead | 773 |
| 062_AML_056 | no | PB | 4 | CR | CR | dead | 77 |
| 075_AML_057 | yes | BM | 1 | ED | ED | dead | 32 |
| 044_AML_058 | no | PB | NA | ED | NA | dead | 12 |
| 019_AML_059 | no | PB | 5 | PR | CR | alive | 1418 |
| 088_AML_060 | no | PB | 3 | CR | Relapse | dead | 305 |
| 043_AML_062 | no | BM | NA | RD | NA | dead | 126 |
| 048_AML_064 | no | PB | 4 | PR | TD | dead | 98 |
| 016_AML_065 | NA | PB | 4 | CR | CR | alive | 1597 |
| 049_AML_068 | no | BM | NA | CR | CR | alive | 1566 |
| 065_AML_069 | no | PB | 5 | CR | CR | alive | 1227 |
| 091_AML_007 | no | PB | 2 | RD | RD | dead | 137 |
| 021_AML_070 | no | BM | 4 | CR | 2REZ | alive | 1019 |
| 080_AML_071 | no | PB | 1 | CR | Relapse | dead | 633 |
| 067_AML_072 | no | PB | 5 | RD | Relapse | dead | 300 |
| 090_AML_073 | yes | PB | 4 | RD | Relapse | dead | 180 |
| 060_AML_074 | no | PB | 4 | RD | RD | dead | 519 |
| 017_AML_077 | no | BM | 4 | CR | TD | dead | 45 |
| 046_AML_078 | NA | BM | NA | ED | ED | dead | 22 |
| 039_AML_079 | no | PB | 4 | PR | CR | alive | 1918 |
| 092_AML_008 | no | BM | 2 | RD | RD | dead | 237 |
| 015_AML_081 | no | PB | 4 | CR | CR | alive | 1707 |
| 064_AML_083 | no | BM | 5 | CR | CR | alive | 1161 |
| 070_AML_084 | no | BM | 4 | CR | Relapse | dead | 515 |
| 034_AML_085 | yes | PB | 5 | CR | Relapse | dead | 1236 |
| 037_AML_086 | NA | PB | 3 | PR | CR | alive | 2007 |
| 027_AML_087 | yes | PB | 1 | RD | RD | dead | 320 |
| 072_AML_088 | yes | PB | 5 | RD | CR | dead | 339 |
| 076_AML_089 | no | BM | 4 | CR | CR | alive | 1195 |
| 029_AML_009 | no | PB | 4 | PR | CR | alive | 896 |
| 074_AML_090 | no | BM | 4 | CR | 2CR | alive | 1262 |
| 045_AML_091 | NA | BM | 4 | PR | Relapse | dead | 95 |
| 036_AML_092 | NA | PB | 4 | RD | RD | dead | 46 |
| 052_AML_093 | NA | PB | 4 | PR | Relapse | dead | 206 |
| 023_AML_094 | NA | BM | 4 | CR | CR | alive | 1134 |
| 089_AML_095 | no | BM | 3 | CR | CR | alive | 883 |
| 035_AML_097 | no | PB | 4 | CR | CR | alive | 2142 |
| 081_AML_098 | no | PB | 2 | ED | ED | dead | 16 |
| 068_AML_099 | NA | BM | NA | CR | CR | alive | 1127 |
| 1_98PB199 | no | PB | 0 | CR | CR | alive | 2177 |
| 2_98PB287 | no | PB | 3 | CR | CR | alive | 2193 |
| 5_98PB834 | no | PB | 5 | ED | ED | dead | 19 |
| 9_98KM1178 | no | BM | 2 | PR | CR | alive | 1928 |
| 12_99PB303 | no | BM | 3 | ED | NA | dead | 25 |
| 13_99PB312 | no | PB | 4 | CR | Relapse | dead | 312 |
| 15_99KM563 | no | PB | 2 | CR | NA | dead | 229 |
| 16_99KM831 | no | BM | 1 | CR | CR | alive | 1734 |
| 17_99KM859 | no | PB | 3 | ED | ED | dead | 4 |
| 18_99KM915 | no | BM | 5 | CR | Relapse | dead | 165 |
| 20_99PB1142 | no | PB | 2 | CR | Relapse | dead | 277 |
| 21_99KM1241 | no | BM | 5 | PR | TD | dead | 252 |
| 22_99PB1321 | no | PB | 0 | RD | Relapse | dead | 197 |
| 23_99PB1458 | no | PB | 1 | CR | Relapse | dead | 438 |
| 26_00PB285 | no | PB | 1 | CR | 2CR | alive | 1266 |
| 27_00KM331 | no | BM | 2 | CR | CR | alive | 1466 |
| 28_00PB369 | NA | PB | 4 | CR | | dead | 660 |
| 29_00PB262 | no | PB | 2 | CR | CR | dead | 703 |
| 31_00PB785 | no | PB | 4 | PR | NA | dead | 402 |
| 32_00KM856 | yes | BM | 0 | RD | CR | dead | 626 |
| 34_00KM1115 | no | BM | 4 | CR | CR | alive | 1331 |
| 37_00PB1729 | no | PB | 1 | PR | CR | alive | 1206 |
| 38_00KM2221 | no | BM | 3 | PR | CR | alive | 1089 |
| 39_00KM2370 | no | BM | 5 | CR | Relapse | dead | 546 |
| 40_00KM2459 | NA | BM | 2 | PR | Relapse | dead | 433 |
| 41_01KM321 | no | BM | 2 | CR | CR | alive | 1159 |
| 42_01KM376 | yes | PB | 2 | RD | RD | dead | 162 |
| 43_01PB382 | no | PB | 3 | CR | TD | dead | 98 |
| 44_01KM459 | no | BM | 6 | ED | NA | dead | 29 |
| 46_01KM452 | no | BM | 7 | CR | CR | alive | 378 |
| 47_01KM496 | no | BM | 1 | RD | Relapse | alive | 519 |
| 48_01KM637 | no | PB | 2 | CR | CR | dead | 470 |
| 49_01PB694 | no | PB | 4 | ED | NA | dead | 31 |
| 50_01KM849 | no | BM | 4 | CR | CR | alive | 476 |
| 51_01KM914 | no | BM | 4 | CR | CR | alive | 1112 |
| 53_01PB1072 | no | PB | 5 | RD | NA | dead | 123 |
| 54_01PB1111 | no | PB | 4 | CR | CR | alive | 994 |
| 56_01KM1105 | no | PB | 1 | CR | Relapse | dead | 231 |
| 57_01PB1172 | no | BM | 5 | CR | Relapse | dead | 306 |
| 58_01KM1273 | no | BM | NA | RD | CR | alive | 1023 |
| 59_01PB1332 | no | PB | 1 | CR | 2CR | alive | 1063 |
| 60_01KM1396 | no | BM | 2 | CR | CR | alive | 1057 |
| 61_01KM1523 | no | BM | 3 | CR | 2CR | alive | 1042 |
| 62_01PB1806 | no | PB | 4 | CR | CR | alive | 1007 |
| 63_01KM1951 | no | BM | 4 | CR | CR | alive | 975 |
| 64_01KM1983 | no | BM | 1 | RD | CR | dead | 249 |
| 66_01KM2148 | no | BM | 0 | PR | refractory | dead | 233 |
| 67_01PB2129 | yes | PB | 3 | CR | CR | alive | 868 |
| 68_01KM2189 | no | PB | 2 | CR | CR | alive | 965 |
| 69_01KM2331 | yes | BM | 1 | RD | NA | dead | 49 |
| 70_01KM2472 | no | PB | 3 | CR | CR | alive | 839 |
| 71_01KM2509 | no | BM | 2 | PR | CR | alive | 908 |
| 74_01KM2551 | no | BM | 3 | ED | NA | dead | 25 |
| 75_01KM2605 | no | BM | 5 | RD | Relapse | dead | 429 |
| 76_01PB2625 | no | PB | 1 | ED | NA | dead | 3 |
| 77_01KM2621 | no | PB | 4 | ED | NA | dead | 19 |
| 79_01PB2846 | no | PB | 5 | CR | Relapse | dead | 442 |
| 80_01KM3019 | no | BM | 5 | CR | Relapse | dead | 271 |
| 81_01KM3062 | no | BM | 1 | PR | Relapse | dead | 399 |
| 82_02KM4 | no | BM | 5 | CR | Relapse | dead | 242 |
| 83_02KM90 | no | PB | 3 | CR | CR | alive | 780 |
| 84_02KM183 | NA | PB | NA | CR | CR | alive | 792 |
| 85_02KM255 | no | BM | 2 | RD | RD | dead | 162 |
| 86_02KM421 | no | PB | 4 | RD | CR | dead | 729 |
| 87_02KM429 | no | BM | 3 | CR | CR | alive | 730 |
| 88_02KM706 | no | PB | 2 | CR | CR | alive | 782 |
| 90_02PB786 | no | PB | 2 | CR | CR | alive | 753 |
| 92_02KM883 | NA | BM | 1 | PR | CR | alive | 739 |
| 93_02KM896 | no | BM | 5 | CR | Relapse | alive | 736 |
| 94_02PB1130 | no | PB | 1 | RD | NA | dead | 26 |
| 95_02KM1236 | no | BM | 1 | RD | Relapse | dead | 576 |
| 96_02KM1407 | no | PB | 2 | ED | NA | dead | 64 |
| 97_02KM1414 | NA | PB | 4 | CR | Relapse | dead | 404 |
| 98_02KM1514 | no | PB | 3 | CR | Relapse | dead | 288 |
| 99_02KM1567 | no | PB | 4 | CR | CR | alive | 657 |
| 100_02KM1573 | no | PB | 1 | CR | CR | alive | 608 |
| 101_02KM1644 | no | BM | 1 | CR | CR | dead | 320 |
| 102_02KM1746 | no | BM | 1 | PR | | alive | 48 |
| 103_02KM1932 | no | BM | 0 | RD | Relapse | dead | 401 |
| 104_02KM2018 | no | BM | 4 | CR | 2CR | alive | 588 |
| 105_02KM2037 | no | PB | 4 | CR | CR | alive | 547 |
| 106_02KM2061 | no | BM | 4 | PR | Relapse | dead | 179 |
| 107_02KM2188 | no | BM | 4 | CR | CR | alive | 608 |
| 108_02KM2242 | no | PB | 1 | RD | CR | dead | 277 |
| 110_02PB2391 | no | PB | 5 | PR | 2Relapse | alive | 585 |
| 111_02KM2472 | no | PB | 4 | CR | CR | alive | 156 |
| 112_02KM2564 | no | PB | 1 | ED | NA | dead | 21 |
| 113_02KM2620 | no | BM | 4 | CR | CR | alive | 573 |
| 114_02KM2732 | no | BM | 1 | PR | 2CR | alive | 510 |
| 115_02PB2822 | no | PB | 4 | PR | | dead | 199 |
| 116_02PB3234 | no | PB | 3 | ED | NA | dead | 12 |
| 117_98PB589 | no | PB | 4 | CR | CR | alive | 2111 |
| 118_98KM785 | no | BM | NA | CR | CR | dead | 736 |
| 119_98KM795 | yes | BM | 3 | ED | ED | dead | 49 |
| 120_98KM798 | no | PB | 2 | CR | CR | alive | 2125 |
| 121_99KM334 | no | BM | 4 | CR | CR | alive | 1718 |
| 122_99KM703 | yes | BM | 3 | ED | NA | dead | 20 |
| 123_99KM723 | no | BM | 1 | RD | RD | dead | 107 |
| 124_99KM1005 | no | BM | 0 | RD | RD | dead | 24 |
| 125_00PB361 | no | PB | 4 | RD | PR | dead | 218 |

The entirety of each patent, patent application, publication and document referenced herein hereby is incorporated by reference, including all tables, drawings, and figures. All patents and publications are herein incorporated by reference to the same extent as if each was specifically and individually indicated to be incorporated by reference. Citation of the above patents, patent applications, publications and documents is not an admission that any of the foregoing is pertinent prior art, nor does it constitute any admission as to the contents or date of these publications or documents. All patents and publications mentioned herein are indicative of the skill levels of those of ordinary skill in the art to which the invention pertains.

Modifications may be made to the foregoing without departing from the scope, spirit and basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. One skilled in the art readily appreciates that the present invention is well adapted to carry out the objects and obtain the ends and advantages mentioned, as well as those inherent therein. The examples provided herein are representative of specific embodiments, are exemplary, and are not intended as limitations on the scope of the invention.

The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention. Embodiments of the invention are set forth in the following claims.

What is claimed is:

1. A method for determining an AML prognosis for a subject, comprising:
(a) determining the methylation state of a target gene in a nucleic acid from the subject; and
(b) comparing the methylation state of (a) to methylation states of the target gene in nucleic acids from subjects having known AML outcomes; whereby the AML prognosis for the subject is determined from step (b); wherein the target gene comprises a sequence from KIAA1447.

2. The method of claim 1, wherein target gene region within the target gene is analyzed, the target gene region comprises a sequence from chromosome 17 positions 77042327-77043930.

3. A method for determining an AML prognosis for a subject, comprising:
(a) determining the methylation state of a target gene in a nucleic acid from the subject; and
(b) comparing the methylation state of (a) to methylation states of the target gene in nucleic acids from subjects having known AML outcomes; whereby the AML prognosis for the subject is determined from step (b); wherein the target gene comprises a sequence from ZD52F10.

4. The method of claim 3, wherein target gene region within the target gene is analyzed, the target gene region comprises a sequence from chromosome 19 positions 40715824-40716843.

5. A method for determining an AML prognosis for a subject, comprising:
(a) determining the methylation state of a target gene in a nucleic acid from the subject; and
(b) comparing the methylation state of (a) to methylation states of the target gene in nucleic acids from subjects having known AML outcomes; whereby the AML prognosis for the subject is determined from step (b); wherein the target gene comprises a sequence from HOXA1.

6. The method of claim 5, wherein target gene region within the target gene is analyzed, the target gene region comprises a sequence from chromosome 7 positions 27109607-27110104.

7. A method for determining an AML prognosis for a subject, comprising:
(a) determining the methylation state of a target gene in a nucleic acid from the subject; and
(b) comparing the methylation state of (a) to methylation states of the target gene in nucleic acids from subjects having known AML outcomes; whereby the AML prognosis for the subject is determined from step (b); wherein the target gene comprises a sequence from PITX2.

8. The method of claim 7, wherein target gene region within the target gene is analyzed, the target gene region comprises a sequence from chromosome 4 positions 111761312-111764113.

9. A method for determining an AML prognosis for a subject, comprising:
(a) determining the methylation state of a target gene in a nucleic acid from the subject; and
(b) comparing the methylation state of (a) to methylation states of the target gene in nucleic acids from subjects having known AML outcomes; whereby the AML prognosis for the subject is determined from step (b); wherein the target gene comprises a sequence from RUNX3.

10. The method of claim 9, wherein target gene region within the target gene is analyzed, the target gene region comprises a sequence from chromosome 1 positions 25127915-25131792.

11. A method for determining an AML prognosis for a subject, comprising:
(a) determining the methylation state of a target gene in a nucleic acid from the subject; and
(b) comparing the methylation state of (a) to methylation states of the target gene in nucleic acids from subjects having known AML outcomes; whereby the AML prognosis for the subject is determined from step (b); wherein the target gene comprises a sequence from NFKbeta1.

12. The method of claim 11, wherein target gene regions within the target gene are analyzed, and the target gene regions comprise a sequence from a region selected from the group consisting of chromosome 4 positions 103640925-103642461 or chromosome 4 positions 103641494-103642135.

13. A method for determining an AML prognosis for a subject, comprising:
(a) determining the methylation state of a target gene in a nucleic acid from the subject; and
(b) comparing the methylation state of (a) to methylation states of the target gene in nucleic acids from subjects having known AML outcomes; whereby the AML prognosis for the subject is determined from step (b); wherein the target gene comprises a sequence from ACTG1.

14. The method of claim 13, wherein target gene regions within the target gene are analyzed, and the target gene region comprises a sequence from a region selected from the group consisting of chromosome 17 positions 77042426-77043830, chromosome 17 positions 77080311-77081236, chromosome 17 positions 77092731-77097121, chromosome 17 positions 77109501-77110986 and chromosome 17 positions 77042426-77043830.

15. A method for determining an AML prognosis for a subject, comprising:
(a) determining the methylation state of a target gene in a nucleic acid from the subject; and
(b) comparing the methylation state of (a) to methylation states of the target gene in nucleic acids from subjects having known AML outcomes; whereby the AML prognosis for the subject is determined from step (b); wherein the target gene comprises a sequence from CDH1.

16. The method of claim 15, wherein target gene region within the target gene is analyzed, the target gene region comprises a sequence from chromosome 16 positions 67328436-67329945.

17. A method for determining an AML prognosis for a subject, comprising:
(a) determining the methylation state of a target gene in a nucleic acid from the subject; and
(b) comparing the methylation state of (a) to methylation states of the target gene in nucleic acids from subjects having known AML outcomes; whereby the AML prognosis for the subject is determined from step (b); wherein the target gene comprises a sequence from DUSP4.

18. The method of claim 17, wherein target gene region within the target gene is analyzed, the target gene region comprises a sequence from chromosome 8 positions 29261385-29265966.

19. A method for determining an AML prognosis for a subject, comprising:
(a) determining the methylation state of a target gene in a nucleic acid from the subject; and
(b) comparing the methylation state of (a) to methylation states of the target gene in nucleic acids from subjects having known AML outcomes; whereby the AML prognosis for the subject is determined from step (b); wherein the target gene comprises a sequence from FARP1.

20. The method of claim 19, wherein target gene region within the target gene is analyzed, the target gene region comprises a sequence from chromosome 13 positions 97592201-97594442.
